SEQUENCE LISTING (284 sequences)

SEQ ID NO: 1 - protein, 7 aa, synthetic construct
SIPYYWG

SEQ ID NO: 2 - protein, 16 aa, synthetic construct
SIYPSGSTDYNPSLKS

SEQ ID NO: 3 - protein, 13 aa, synthetic construct
GGARDRIDHAFSI

SEQ ID NO: 4 - protein, 13 aa, synthetic construct
GGARDRIDHAFEI

SEQ ID NO: 5 - protein, 13 aa, synthetic construct
GGARDRIDHAFDI

SEQ ID NO: 6 - protein, 11 aa, synthetic construct
QASQSISNYLN

SEQ ID NO: 7 - protein, 11 aa, synthetic construct
QASQDISNYLN

SEQ ID NO: 8 - protein, 7 aa, synthetic construct
DASNSET

SEQ ID NO: 9 - protein, 7 aa, synthetic construct
DASNLET

SEQ ID NO: 10 - protein, 8 aa, synthetic construct
QQYYDLYK

SEQ ID NO: 11 - protein, 8 aa, synthetic construct
QQYYDLYT

SEQ ID NO: 12 - protein, 10 aa, synthetic construct
GGSISSIPYY

SEQ ID NO: 13 - protein, 7 aa, synthetic construct
IYPSGST

SEQ ID NO: 14 - protein, 15 aa, synthetic construct
ARGGARDRIDHAFSI

SEQ ID NO: 15 - protein, 15 aa, synthetic construct
ARGGARDRIDHAFEI

SEQ ID NO: 16 - protein, 15 aa, synthetic construct
ARGGARDRIDHAFDI

SEQ ID NO: 17 - protein, 6 aa, synthetic construct
QSISNY

SEQ ID NO: 18 - protein, 6 aa, synthetic construct
QDISNY

SEQ ID NO: 19 - skipped

SEQ ID NO: 20 - protein, 8 aa, synthetic construct
QQYYDLYK

SEQ ID NO: 21 - protein, 8 aa, synthetic construct
QQYYDLYT

SEQ ID NO: 22 - protein, 7 aa, synthetic construct
SESYYWG

SEQ ID NO: 23 - protein, 16 aa, synthetic construct
SIYYSGSTDYNPSLKS

SEQ ID NO: 24 - protein, 13 aa, synthetic construct
GGASDRIDKAFEI

SEQ ID NO: 25 - protein, 13 aa, synthetic construct
GGASDRIDHAFEI

SEQ ID NO: 26 - protein, 13 aa, synthetic construct
GGASDRIDHAFSI

SEQ ID NO: 27 - protein, 13 aa, synthetic construct
GGASDRIDHAFDI

SEQ ID NO: 28 - protein, 10 aa, synthetic construct
GGSISSESYY

SEQ ID NO: 29 - protein, 7 aa, synthetic construct
IYYSGST

SEQ ID NO: 30 - protein, 15 aa, synthetic construct
ARGGASDRIDKAFEI

SEQ ID NO: 31 - protein, 15 aa, synthetic construct
ARGGASDRIDHAFEI

SEQ ID NO: 32 - protein, 15 aa, synthetic construct
ARGGASDRIDHAFSI

SEQ ID NO: 33 - protein, 15 aa, synthetic construct
ARGGASDRIDHAFDI

SEQ ID NO: 34 - protein, 123 aa, synthetic construct
QLQLQESGPGLKKPSETLSLTCTVSGGSISSIPYYWGWIREPPGKGLEWIGSIYPSGSTDYNPSLKSRVTISVDTSKNQFSLKLSSVTAADTAVYYCARGGARDRIDHAFSIWGQGTMVTVSS

SEQ ID NO: 35 - protein, 106 aa, synthetic construct
DIQMTQSPSSLSASVGDRVTITCQASQSISNYLNWYQKKPGKAPKLLIYDASNSETGVPSRFSGSGSGTDFTFTISSLQPEDIATYYCQQYYDLYKFGGGTKVEIK

SEQ ID NO: 36 - protein, 123 aa, synthetic construct
QLQLQESGPGLKKPSETLSLTCTVSGGSISSIPYYWGWIRKPPGKGLEWIGSIYPSGSTDYNPSLKSRVTISVDTSKNQFSLKLSSVTAADTAVYYCARGGARDRIDHAFSIWGQGTMVTVSS

SEQ ID NO: 37 - protein, 106 aa, synthetic construct
DIQMTQSPSSLSASVGDRVTITCQASQSISNYLNWYQEKPGKAPKLLIYDASNSETGVPSRFSGSGSGTDFTFTISSLQPEDIATYYCQQYYDLYKFGGGTKVEIK

SEQ ID NO: 38 - protein, 123 aa, synthetic construct
QLQLQESGPGLKKPSETLSLTCTVSGGSISSIPYYWGWIRQPPGKGLEWIGSIYPSGSTDYNPSLKSRVTISVDTSKNQFSLKLSSVTAADTAVYYCARGGARDRIDHAFSIWGQGTMVTVSS

SEQ ID NO: 39 - protein, 106 aa, synthetic construct
DIQMTQSPSSLSASVGDRVTITCQASQSISNYLNWYQQKPGKAPKLLIYDASNSETGVPSRFSGSGSGTDFTFTISSLQPEDIATYYCQQYYDLYKFGGGTKVEIK

SEQ ID NO: 40 - protein, 123 aa, synthetic construct
QLQLQESGPGLVKPSETLSLTCTVSGGSISSIPYYWGWIRKPPGKGLEWIGSIYPSGSTDYNPSLKSRVTISVDTSKNQFSLKLSSVTAADTAVYYCARGGARDRIDHAFSIWGQGTMVTVSS

SEQ ID NO: 41 - protein, 106 aa, synthetic construct
DIQMTQSPSSLSASVGDRVTITCQASQDISNYLNWYQEKPGKAPKLLIYDASNLETGVPSRFSGSGSGTDFTFTISSLQPEDIATYYCQQYYDLYTFGGGTKVEIK

SEQ ID NO: 42 - protein, 123 aa, synthetic construct
QLQLQESGPGLVKPSETLSLTCTVSGGSISSIPYYWGWIRQPPGKGLEWIGSIYPSGSTDYNPSLKSRVTISVDTSKNQFSLKLSSVTAADTAVYYCARGGARDRIDHAFSIWGQGTMVTVSS

SEQ ID NO: 43 - protein, 106 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYYDLYTFGGGTKVEIK

SEQ ID NO: 44 - protein, 123 aa, synthetic construct
QLQLQESGPGLVKPSETLSLTCTVSGGSISSIPYYWGWIRQPPGKGLEWIGSIYPSGSTDYNPSLKSRVTISVDTSKNQFSLKLSSVTAADTAVYYCARGGARDRIDHAFEIWGQGTMVTVSS

SEQ ID NO: 45 - protein, 123 aa, synthetic construct
QLQLQESGPGLVKPSETLSLTCTVSGGSISSIPYYWGWIRQPPGKGLEWIGSIYPSGSTDYNPSLKSRVTISVDTSKNQFSLKLSSVTAADTAVYYCARGGARDRIDHAFDIWGQGTMVTVSS

SEQ ID NO: 46 - protein, 123 aa, synthetic construct
QLQLQESGPGLKKPSETLSLTCTVSGGSISSESYYWGWIRKPPGKGLEWIGSIYYSGSTDYNPSLKSRVTISVDTSKNQFSLKLSSVTAADTAVYYCARGGASDRIDKAFEIWGQGTMVTVSS

SEQ ID NO: 47 - protein, 106 aa, synthetic construct; identical to SEQ ID NO: 37

SEQ ID NO: 48 - protein, 123 aa, synthetic construct
QLQLQESGPGLKKPSETLSLTCTVSGGSISSESYYWGWIRQPPGKGLEWIGSIYYSGSTDYNPSLKSRVTISVDTSKNQFSLKLSSVTAADTAVYYCARGGASDRIDKAFEIWGQGTMVTVSS

SEQ ID NO: 49 - protein, 123 aa, synthetic construct
QLQLQESGPGLVKPSETLSLTCTVSGGSISSESYYWGWIRKPPGKGLEWIGSIYYSGSTDYNPSLKSRVTISVDTSKNQFSLKLSSVTAADTAVYYCARGGASDRIDKAFEIWGQGTMVTVSS

SEQ ID NO: 50 - protein, 123 aa, synthetic construct
QLQLQESGPGLVKPSETLSLTCTVSGGSISSESYYWGWIRQPPGKGLEWIGSIYYSGSTDYNPSLKSRVTISVDTSKNQFSLKLSSVTAADTAVYYCARGGASDRIDKAFEIWGQGTMVTVSS

SEQ ID NO: 51 - protein, 123 aa, synthetic construct
QLQLQESGPGLKKPSETLSLTCTVSGGSISSESYYWGWIRKPPGKGLEWIGSIYYSGSTDYNPSLKSRVTISVDTSKNQFSLKLSSVTAADTAVYYCARGGASDRIDHAFEIWGQGTMVTVSS

SEQ ID NO: 52 - protein, 123 aa, synthetic construct
QLQLQESGPGLKKPSETLSLTCTVSGGSISSESYYWGWIRQPPGKGLEWIGSIYYSGSTDYNPSLKSRVTISVDTSKNQFSLKLSSVTAADTAVYYCARGGASDRIDHAFEIWGQGTMVTVSS

SEQ ID NO: 53 - protein, 123 aa, synthetic construct
QLQLQESGPGLVKPSETLSLTCTVSGGSISSESYYWGWIRKPPGKGLEWIGSIYYSGSTDYNPSLKSRVTISVDTSKNQFSLKLSSVTAADTAVYYCARGGASDRIDHAFEIWGQGTMVTVSS

SEQ ID NO: 54 - protein, 123 aa, synthetic construct
QLQLQESGPGLVKPSETLSLTCTVSGGSISSESYYWGWIRQPPGKGLEWIGSIYYSGSTDYNPSLKSRVTISVDTSKNQFSLKLSSVTAADTAVYYCARGGASDRIDHAFEIWGQGTMVTVSS

SEQ ID NO: 55 - protein, 123 aa, synthetic construct
QLQLQESGPGLVKPSETLSLTCTVSGGSISSESYYWGWIRQPPGKGLEWIGSIYYSGSTDYNPSLKSRVTISVDTSKNQFSLKLSSVTAADTAVYYCARGGASDRIDHAFSIWGQGTMVTVSS

SEQ ID NO: 56 - protein, 123 aa, synthetic construct
QLQLQESGPGLVKPSETLSLTCTVSGGSISSESYYWGWIRQPPGKGLEWIGSIYYSGSTDYNPSLKSRVTISVDTSKNQFSLKLSSVTAADTAVYYCARGGASDRIDHAFDIWGQGTMVTVSS

SEQ ID NO: 57 - protein, 5 aa, synthetic construct
SYAMS

SEQ ID NO: 58 - protein, 17 aa, synthetic construct
AISGSGGSTYYADSVKG

SEQ ID NO: 59 - protein, 18 aa, synthetic construct
EYYYDPLQYYHYYYGMDV

SEQ ID NO: 60 - protein, 11 aa, synthetic construct
SGDALPKQYAY

SEQ ID NO: 61 - protein, 7 aa, synthetic construct
KDSERPS

SEQ ID NO: 62 - protein, 11 aa, synthetic construct
QSADSSGTYWV

SEQ ID NO: 63 - protein, 8 aa, synthetic construct
GFTFSSYA

SEQ ID NO: 64 - protein, 8 aa, synthetic construct
ISGSGGST

SEQ ID NO: 65 - protein, 20 aa, synthetic construct
AREYYYDPLQYYHYYYGMDV

SEQ ID NO: 66 - protein, 20 aa, synthetic construct
AREYYYDPLNTYHYYYGMDV

SEQ ID NO: 67 - protein, 6 aa, synthetic construct
ALPKQY

SEQ ID NO: 68 - skipped

SEQ ID NO: 69 - protein, 11 aa, synthetic construct
QSADSSGTYWV

SEQ ID NO: 70 - protein, 127 aa, synthetic construct
EVQLLESGGGLVQPGGSLRLSCAASGFTFSSYAMSWVREAPGKGLEWVSAISGSGGSTYYADSVKGRFTISRDNSKNTLYLQMNSLRAEDTAVYYCAREYYYDPLQYYHYYYGMDVWGQGTTVTVSS

SEQ ID NO: 71 - protein, 108 aa, synthetic construct
SYELTQPPSVSVSPGQTARITCSGDALPKQYAYWYQKKPGQAPVLVIYKDSERPSGIPERFSGSSKGTTVTLTISGVQAEDEADYYCQSADSSGTYWVFGGGTKLTVL

SEQ ID NO: 72 - protein, 127 aa, synthetic construct
EVQLLESGGGLVQPGGSLRLSCAASGFTFSSYAMSWVRKAPGKGLEWVSAISGSGGSTYYADSVKGRFTISRDNSKNTLYLQMNSLRAEDTAVYYCAREYYYDPLQYYHYYYGMDVWGQGTTVTVSS

SEQ ID NO: 73 - protein, 108 aa, synthetic construct
SYELTQPPSVSVSPGQTARITCSGDALPKQYAYWYQEKPGQAPVLVIYKDSERPSGIPERFSGSSKGTTVTLTISGVQAEDEADYYCQSADSSGTYWVFGGGTKLTVL

SEQ ID NO: 74 - protein, 127 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAREYYYDPLQYYHYYYGMDVWGQGTTVTVSS

SEQ ID NO: 75 - protein, 108 aa, synthetic construct
SYELTQPPSVSVSPGQTARITCSGDALPKQYAYWYQQKPGQAPVLVIYKDSERPSGIPERFSGSSKGTTVTLTISGVQAEDEADYYCQSADSSGTYWVFGGGTKLTVL

SEQ ID NO: 76 - protein, 127 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAREYYYDPLNTYHYYYGMDVWGQGTTVTVSS

SEQ ID NO: 77 - protein, 108 aa, synthetic construct
SYELMQPPSVSVSPGQTARITCSGDALPKQYAYWYQQKPGQAPVLVIYKDSERPSGIPERFSGSSSGTTVTLTISGVQAEDEADYYCQSADSSGTYWVFGGGTKLTVL

SEQ ID NO: 78 - protein, 5 aa, synthetic construct
SYAMT

SEQ ID NO: 79 - protein, 17 aa, synthetic construct
DISGSGRSIFYADSVKG

SEQ ID NO: 80 - protein, 15 aa, synthetic construct
DLGFCSGASCNEADY

SEQ ID NO: 81 - protein, 11 aa, synthetic construct
RASQGIRNDLD

SEQ ID NO: 82 - protein, 7 aa, synthetic construct
ATSGLQS

SEQ ID NO: 83 - protein, 9 aa, synthetic construct
LQHSRYPWT

SEQ ID NO: 84 - protein, 8 aa, synthetic construct
GFTFSSYA

SEQ ID NO: 85 - protein, 8 aa, synthetic construct
GFTFNSYA

SEQ ID NO: 86 - protein, 8 aa, synthetic construct
ISGSGRSI

SEQ ID NO: 87 - protein, 17 aa, synthetic construct
AKDLGFCSGASCNEADY

SEQ ID NO: 88 - protein, 6 aa, synthetic construct
QGIRND

SEQ ID NO: 89 - skipped

SEQ ID NO: 90 - protein, 9 aa, synthetic construct
LQHSRYPWT

SEQ ID NO: 91 - protein, 124 aa, synthetic construct
EVQLVESGGGFVQPGMSLRLSCAASGFTFSSYAMTWVRKAPGKGLEWVSDISGSGRSIFYADSVKGRFTISRDNSKRTLYLQMNSLRAEDTAIYYCAKDLGFCSGASCNEADYWGQGTLVTVSS

SEQ ID NO: 92 - protein, 107 aa, synthetic construct
DIQMTQSPSSLSASVGDRVTITCRASQGIRNDLDWYQEKPGKAPKRLIYATSGLQSGVPSRFSGSGSGTEFTLTISSLQPEDFATYYCLQHSRYPWTFGQGTKVEIK

SEQ ID NO: 93 - protein, 124 aa, synthetic construct
EVQLVESGGGFVQPGMSLRLSCAASGFTFSSYAMTWVREAPGKGLEWVSDISGSGRSIFYADSVKGRFTISRDNSKRTLYLQMNSLRAEDTAIYYCAKDLGFCSGASCNEADYWGQGTLVTVSS

SEQ ID NO: 94 - protein, 107 aa, synthetic construct
DIQMTQSPSSLSASVGDRVTITCRASQGIRNDLDWYQKKPGKAPKRLIYATSGLQSGVPSRFSGSGSGTEFTLTISSLQPEDFATYYCLQHSRYPWTFGQGTKVEIK

SEQ ID NO: 95 - protein, 124 aa, synthetic construct
EVQLVESGGGFVQPGMSLRLSCAASGFTFSSYAMTWVRHAPGKGLEWVSDISGSGRSIFYADSVKGRFTISRDNSKRTLYLQMNSLRAEDTAIYYCAKDLGFCSGASCNEADYWGQGTLVTVSS

SEQ ID NO: 96 - protein, 107 aa, synthetic construct
DIQMTQSPSSLSASVGDRVTITCRASQGIRNDLDWYQQKPGKAPKRLIYATSGLQSGVPSRFSGSGSGTEFTLTISSLQPEDFATYYCLQHSRYPWTFGQGTKVEIK

SEQ ID NO: 97 - protein, 124 aa, synthetic construct
EVQLVESGGGFVQPGMSLRLSCAASGFTFNSYAMTWVRHAPGKGLEWVSDISGSGRSIFYADSVKGRFTISRDNSKRTLYLQMNSLRAEDTAIYYCAKDLGFCSGASCNEADYWGQGTLVTVSS

SEQ ID NO: 98 - protein, 341 aa, synthetic construct
SYELTQPPSVSVSPGQTARITCSGDALPKQYAYWYQKKPGQAPVLVIYKDSERPSGIPERFSGSSKGTTVTLTISGVQAEDEADYYCQSADSSGTYWVFGGGTKLTVLGGGGSGGGGSDIQMTQSPSSLSASVGDRVTITCQASQSISNYLNWYQKKPGKAPKLLIYDASNSETGVPSRFSGSGSGTDFTFTISSLQPEDIATYYCQQYYDLYKFGGGTKVEIKGGGGSGGGGSRTVAAPAVFIFPPSKKQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 579 aa, synthetic construct
QLQLQESGPGLKKPSETLSLTCTVSGGSISSIPYYWGWIREPPGKGLEWIGSIYPSGSTDYNPSLKSRVTISVDTSKNQFSLKLSSVTAADTAVYYCARGGARDRIDHAFSIWGQGTMVTVSSEVQLLESGGGLVQPGGSLRLSCAASGFTFSSYAMSWVREAPGKGLEWVSAISGSGGSTYYADSVKGRFTISRDNSKNTLYLQMNSLRAEDTAVYYCAREYYYDPLQYYHYYYGMDVWGQGTTVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDEKVEPDSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 100 - protein, 453 aa, synthetic construct
EVQLVESGGGFVQPGMSLRLSCAASGFTFSSYAMTWVRKAPGKGLEWVSDISGSGRSIFYADSVKGRFTISRDNSKRTLYLQMNSLRAEDTAIYYCAKDLGFCSGASCNEADYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 101 - protein, 214 aa, synthetic construct
DIQMTQSPSSLSASVGDRVTITCRASQGIRNDLDWYQEKPGKAPKRLIYATSGLQSGVPSRFSGSGSGTEFTLTISSLQPEDFATYYCLQHSRYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 10 aa, synthetic construct
GGGGSGGGGS

SEQ ID NO: 103 - protein, 15 aa, synthetic construct
GGGGSGGGGSGGGGS

SEQ ID NO: 104 - protein, 5 aa, synthetic construct
TKGPS

SEQ ID NO: 105 - protein, 7 aa, synthetic construct
GQPKAAP

SEQ ID NO: 106 - protein, 10 aa, synthetic construct
GGSGSSGSGG

SEQ ID NO: 107 - protein, 19 aa, synthetic construct
MGWSCIILFLVATATGVHS

SEQ ID NO: 108 - protein, 18 aa, synthetic construct
EYYYDPLNTYHYYYGMDV

SEQ ID NO: 109 - protein, 5 aa, synthetic construct
DKTHT

SEQ ID NO: 110 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 111 - protein, 133 aa, synthetic construct
QVSHRYPRIQSIKVQFTEYKKEKGFILTSQKEDEIMKVQDNSVIINCDGFYLISLKGYFSQEVDISLHYQKDEEPLFQLKKVRSVNSLMVASLTYKDKVYLNVTTDNTSLDDFHVNGGELILIHQNPGEFCVL

SEQ ID NO: 112 - protein, 214 aa, synthetic construct
DIQMTQSPSSLSASVGDRVTITCRASQGIRNDLDWYQQKPGKAPKRLIYATSGLQSGVPSRFSGSGSGTEFTLTISSLQPEDFATYYCLQHSRYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 238 aa, synthetic construct
EVQLVESGGGFVQPGMSLRLSCAASGFTFNSYAMTWVRHAPGKGLEWVSDISGSGRSIFYADSVKGRFTISRDNSKRTLYLQMNSLRAEDTAIYYCAKDLGFCSGASCNEADYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTHHHHHH

SEQ ID NO: 114 - protein, 12 aa, synthetic construct
QSLLDSDDGNTY

SEQ ID NO: 115 - skipped

SEQ ID NO: 116 - protein, 9 aa, synthetic construct
MQRIEFPYT

SEQ ID NO: 117 - protein, 8 aa, synthetic construct
GFTFSDYF

SEQ ID NO: 118 - protein, 8 aa, synthetic construct
ISSTGTTK

SEQ ID NO: 119 - protein, 9 aa, synthetic construct
ARDGSSLDS

SEQ ID NO: 120 - protein, 6 aa, synthetic construct
QDIKND

SEQ ID NO: 121 - skipped

SEQ ID NO: 122 - protein, 9 aa, synthetic construct
LQDNNYPRT

SEQ ID NO: 123 - protein, 8 aa, synthetic construct
GGSFDGYY

SEQ ID NO: 124 - protein, 7 aa, synthetic construct
IFHIGGT

SEQ ID NO: 125 - protein, 18 aa, synthetic construct
ARGITMGRGNYYYYGLDV

SEQ ID NO: 126 - protein, 6 aa, synthetic construct
QDIKND

SEQ ID NO: 127 - skipped

SEQ ID NO: 128 - protein, 9 aa, synthetic construct
LQDNNYPRT

SEQ ID NO: 129 - protein, 8 aa, synthetic construct
GGSFDGYY

SEQ ID NO: 130 - protein, 7 aa, synthetic construct
INHIGGT

SEQ ID NO: 131 - protein, 18 aa, synthetic construct
ARGITMGRGNYYYYGLDV

SEQ ID NO: 132 - protein, 116 aa, synthetic construct
EVQLVESGGGLVKPGGSLRLSCAASGFTFSDYFMSWIREAPGKGLEWVSYISSTGTTKYYADSVKGRFTMSRDNAKNSLYLQMSSLRAEDTAVYYCARDGSSLDSWGQGTLVTVSS

SEQ ID NO: 133 - protein, 113 aa, synthetic construct
DIVMTQSPLSLPVTPGEPASISCRSSQSLLDSDDGNTYLDWFLKKPGQSPQLLIYTFFYRASGVPDRFSGSGSGTDFTLKISRVEAEDVGVYYCMQRIEFPYTFGQGTKLEIK

SEQ ID NO: 134 - protein, 124 aa, synthetic construct
QVQLQQWGAGLLKPSETLSLTCAVFGGSFDGYYWNWIREPPGKGLEWIGEIFHIGGTIYNPSLKSRVTISEDTSKNQFSLRLNSVTAADTATYYCARGITMGRGNYYYYGLDVWGQGTPVTVSS

SEQ ID NO: 135 - protein, 107 aa, synthetic construct
DIQMTQSPSSLSASVGDRVTITCRASQDIKNDLGWYQKKPGKAPKFLIFGTYSLQSGVPSRFSGSGSGTEFTLTISSLQPEDFATYYCLQDNNYPRTFGQGTKVEIK

SEQ ID NO: 136 - protein, 124 aa, synthetic construct
QVQLQQWGAGLLKPSETLSLTCAVYGGSFDGYYWNWIREPPGKGLEWIGEINHIGGTIYNPSLKSRVTISEDTSKNQFSLKLSSVTAADTAVYYCARGITMGRGNYYYYGLDVWGQGTTVTVSS

SEQ ID NO: 137 - protein, 107 aa, synthetic construct; identical to SEQ ID NO: 135

SEQ ID NO: 138 - protein, 341 aa, synthetic construct
SYELTQPPSVSVSPGQTARITCSGDALPKQYAYWYQEKPGQAPVLVIYKDSERPSGIPERFSGSSKGTTVTLTISGVQAEDEADYYCQSADSSGTYWVFGGGTKLTVLGGGGSGGGGSDIQMTQSPSSLSASVGDRVTITCQASQDISNYLNWYQEKPGKAPKLLIYDASNLETGVPSRFSGSGSGTDFTFTISSLQPEDIATYYCQQYYDLYTFGGGTKVEIKGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 579 aa, synthetic construct
QLQLQESGPGLVKPSETLSLTCTVSGGSISSESYYWGWIRKPPGKGLEWIGSIYYSGSTDYNPSLKSRVTISVDTSKNQFSLKLSSVTAADTAVYYCARGGASDRIDHAFEIWGQGTMVTVSSEVQLLESGGGLVQPGGSLRLSCAASGFTFSSYAMSWVRKAPGKGLEWVSAISGSGGSTYYADSVKGRFTISRDNSKNTLYLQMNSLRAEDTAVYYCAREYYYDPLQYYHYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 140 - protein, 453 aa, synthetic construct
QVQLQQWGAGLLKPSETLSLTCAVFGGSFDGYYWNWIREPPGKGLEWIGEIFHIGGTIYNPSLKSRVTISEDTSKNQFSLRLNSVTAADTATYYCARGITMGRGNYYYYGLDVWGQGTPVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDEKVEPDSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 141 - protein, 214 aa, synthetic construct
DIQMTQSPSSLSASVGDRVTITCRASQDIKNDLGWYQKKPGKAPKFLIFGTYSLQSGVPSRFSGSGSGTEFTLTISSLQPEDFATYYCLQDNNYPRTFGQGTKVEIKRTVAAPAVFIFPPSKKQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 341 aa, synthetic construct; identical to SEQ ID NO: 138

SEQ ID NO: 143 - protein, 579 aa, synthetic construct
QLQLQESGPGLVKPSETLSLTCTVSGGSISSIPYYWGWIRKPPGKGLEWIGSIYPSGSTDYNPSLKSRVTISVDTSKNQFSLKLSSVTAADTAVYYCARGGARDRIDHAFSIWGQGTMVTVSSEVQLLESGGGLVQPGGSLRLSCAASGFTFSSYAMSWVRKAPGKGLEWVSAISGSGGSTYYADSVKGRFTISRDNSKNTLYLQMNSLRAEDTAVYYCAREYYYDPLQYYHYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 144 - protein, 453 aa, synthetic construct
QVQLQQWGAGLLKPSETLSLTCAVYGGSFDGYYWNWIREPPGKGLEWIGEINHIGGTIYNPSLKSRVTISEDTSKNQFSLKLSSVTAADTAVYYCARGITMGRGNYYYYGLDVWGQGTTVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDEKVEPDSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 145 - protein, 214 aa, synthetic construct; identical to SEQ ID NO: 141

SEQ ID NO: 146 - protein, 341 aa, synthetic construct; identical to SEQ ID NO: 138

SEQ ID NO: 147 - protein, 579 aa, synthetic construct; identical to SEQ ID NO: 143

SEQ ID NO: 148 - protein, 453 aa, synthetic construct; identical to SEQ ID NO: 140

SEQ ID NO: 149 - protein, 214 aa, synthetic construct; identical to SEQ ID NO: 141

SEQ ID NO: 150 - protein, 341 aa, synthetic construct; identical to SEQ ID NO: 138

SEQ ID NO: 151 - protein, 579 aa, synthetic construct; identical to SEQ ID NO: 143

SEQ ID NO: 152 - protein, 445 aa, synthetic construct
EVQLVESGGGLVKPGGSLRLSCAASGFTFSDYFMSWIREAPGKGLEWVSYISSTGTTKYYADSVKGRFTMSRDNAKNSLYLQMSSLRAEDTAVYYCARDGSSLDSWGQGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDEKVEPDSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 153 - protein, 220 aa, synthetic construct
DIVMTQSPLSLPVTPGEPASISCRSSQSLLDSDDGNTYLDWFLKKPGQSPQLLIYTFFYRASGVPDRFSGSGSGTDFTLKISRVEAEDVGVYYCMQRIEFPYTFGQGTKLEIKRTVAAPAVFIFPPSKKQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 238 aa, synthetic construct
QVQLQQWGAGLLKPSETLSLTCAVFGGSFDGYYWNWIRQPPGKGLEWIGEINHIGGTIYNPSLKSRVTISEDTSKNQFSLRLNSVTAADTATYYCARGITMGRGNYYYYGLDVWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTHHHHHH

SEQ ID NO: 155 - protein, 214 aa, synthetic construct
DILMTQSPSSLSASVGDRVTITCRASQDIKNDLGWYQQKPGKVPKFLIFGTYSLQSGVPSRFSGSGSGTDFTLTINSLQPEDFATYYCLQDNNYPRTFGQGTKVGFKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 124 aa, synthetic construct
MGPNSEDKNFPRTVMVNLNIHNRNTNTNPKRSSDYYDRSTSPWNLHRNEDPERYPSVIWEAKCRHLGCINADGNVDYHMNSVPIQQEILVLRREPPHSPNSFRLEKILVSVGCTCVTPIVHHVA

SEQ ID NO: 157 - protein, 8 aa, synthetic construct
GYTFTDYY

SEQ ID NO: 158 - protein, 8 aa, synthetic construct
VDPEDGET

SEQ ID NO: 159 - protein, 24 aa, synthetic construct
ARGIARYPNGPGGLLYYYYYGMDV

SEQ ID NO: 160 - protein, 6 aa, synthetic construct
NIGSKN

SEQ ID NO: 161 - skipped

SEQ ID NO: 162 - protein, 9 aa, synthetic construct
QVWDSFAFV

SEQ ID NO: 163 - protein, 8 aa, synthetic construct
GFTFSSYA

SEQ ID NO: 164 - protein, 8 aa, synthetic construct
ISYDGSNK

SEQ ID NO: 165 - protein, 11 aa, synthetic construct
ARYSNYWWFDY

SEQ ID NO: 166 - protein, 8 aa, synthetic construct
SSNIGNNA

SEQ ID NO: 167 - skipped

SEQ ID NO: 168 - protein, 11 aa, synthetic construct
AAWDDSLSGVV

SEQ ID NO: 169 - protein, 8 aa, synthetic construct
GFTFSSYA

SEQ ID NO: 170 - protein, 8 aa, synthetic construct
ISYDGSNK

SEQ ID NO: 171 - protein, 11 aa, synthetic construct
ARYDNTRIFDY

SEQ ID NO: 172 - protein, 8 aa, synthetic construct
SSNIGNNA

SEQ ID NO: 173 - skipped

SEQ ID NO: 174 - protein, 11 aa, synthetic construct
AAWDDSLNGYV

SEQ ID NO: 175 - protein, 8 aa, synthetic construct
GFTFSSYA

SEQ ID NO: 176 - protein, 8 aa, synthetic construct
ISYDGSNK

SEQ ID NO: 177 - protein, 11 aa, synthetic construct
ARLPWVGSVDY

SEQ ID NO: 178 - protein, 8 aa, synthetic construct
SSNIGNNA

SEQ ID NO: 179 - skipped

SEQ ID NO: 180 - protein, 11 aa, synthetic construct
AAWDDSLFGWV

SEQ ID NO: 181 - protein, 10 aa, synthetic construct
GFSLSTSGVG

SEQ ID NO: 182 - protein, 7 aa, synthetic construct
IYWDDDK

SEQ ID NO: 183 - protein, 15 aa, synthetic construct
AHRTGIAVAGIAFDI

SEQ ID NO: 184 - protein, 6 aa, synthetic construct
KLGDKY

SEQ ID NO: 185 - skipped

SEQ ID NO: 186 - protein, 10 aa, synthetic construct
QAWDSSSLYV

SEQ ID NO: 187 - protein, 10 aa, synthetic construct
GGSISGSSYY

SEQ ID NO: 188 - protein, 7 aa, synthetic construct
IYSSGST

SEQ ID NO: 189 - protein, 15 aa, synthetic construct
AKPLLRDYYFYGVDV

SEQ ID NO: 190 - protein, 6 aa, synthetic construct
QAISSY

SEQ ID NO: 191 - skipped

SEQ ID NO: 192 - protein, 9 aa, synthetic construct
QQLKSYPWT

SEQ ID NO: 193 - protein, 8 aa, synthetic construct
GFTFSSYA

SEQ ID NO: 194 - protein, 8 aa, synthetic construct
ISESGSST

SEQ ID NO: 195 - protein, 17 aa, synthetic construct
AKDRWFEGYHSYYGMDV

SEQ ID NO: 196 - protein, 6 aa, synthetic construct
QGISNN

SEQ ID NO: 197 - skipped

SEQ ID NO: 198 - protein, 10 aa, synthetic construct
QQLKSYPSFT

SEQ ID NO: 199 - protein, 10 aa, synthetic construct
GGSISSSSYY

SEQ ID NO: 200 - protein, 7 aa, synthetic construct
IYSSGST

SEQ ID NO: 201 - protein, 15 aa, synthetic construct
AKPLLRDYYYYGVDV

SEQ ID NO: 202 - protein, 6 aa, synthetic construct
QAISSY

SEQ ID NO: 203 - skipped

SEQ ID NO: 204 - protein, 9 aa, synthetic construct
QQLNSFPWT

SEQ ID NO: 205 - protein, 8 aa, synthetic construct
GYTFTTYG

SEQ ID NO: 206 - protein, 8 aa, synthetic construct
ISVFNGSP

SEQ ID NO: 207 - protein, 14 aa, synthetic construct
ARGGTGDYYPLFDY

SEQ ID NO: 208 - protein, 6 aa, synthetic construct
QGIRND

SEQ ID NO: 209 - skipped

SEQ ID NO: 210 - protein, 9 aa, synthetic construct
LQDYNYPYT

SEQ ID NO: 211 - protein, 10 aa, synthetic construct
GGSITSTSYF

SEQ ID NO: 212 - protein, 7 aa, synthetic construct
IYHSGST

SEQ ID NO: 213 - protein, 13 aa, synthetic construct
ARHEFGIRDAFDI

SEQ ID NO: 214 - protein, 6 aa, synthetic construct
QSIGNN

SEQ ID NO: 215 - skipped

SEQ ID NO: 216 - protein, 9 aa, synthetic construct
HQSNSFPFT

SEQ ID NO: 217 - protein, 131 aa, synthetic construct
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCARGIARYPNGPGGLLYYYYYGMDVWGQGTTVTVSS

SEQ ID NO: 218 - protein, 106 aa, synthetic construct
SYELTQPLSVSVALGQTARITCGGNNIGSKNVHWYQQKPGQAPVLVIYRDSNRPSGIPERFSGSNSGNTATLTISRAQAGDEADYYCQVWDSFAFVFGGGTKLTVL

SEQ ID NO: 219 - protein, 118 aa, synthetic construct
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARYSNYWWFDYWGQGTLVTVSS

SEQ ID NO: 220 - protein, 110 aa, synthetic construct
QSVLTQPPSVSEAPRQRVTISCSGSSSNIGNNAVNWYQQLPGKAPKLLIYYDDLLPSGVSDRFSGSKSGTSASLAISGLQSEDEADYYCAAWDDSLSGVVFGGGTKLTVL

SEQ ID NO: 221 - protein, 118 aa, synthetic construct
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARYDNTRIFDYWGQGTLVTVSS

SEQ ID NO: 222 - protein, 110 aa, synthetic construct
QSVLTQPPSVSEAPRQRVTISCSGSSSNIGNNAVNWYQQLPGKAPKLLIYYDDLLPSGVSDRFSGSKSGTSASLAISGLQSEDEADYYCAAWDDSLNGYVFGGGTKLTVL

SEQ ID NO: 223 - protein, 118 aa, synthetic construct
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARLPWVGSVDYWGQGTLVTVSS

SEQ ID NO: 224 - protein, 110 aa, synthetic construct
QSVLTQPPSVSEAPRQRVTISCSGSSSNIGNNAVNWYQQLPGKAPKLLIYYDDLLPSGVSDRFSGSKSGTSASLAISGLQSEDEADYYCAAWDDSLFGWVFGGGTKLTVL

SEQ ID NO: 225 - protein, 123 aa, synthetic construct
QVTLKESGPTLVKPTQTLTLTCTFSGFSLSTSGVGVGWIRQPPGKALEWLALIYWDDDKRYSPSLESRLTITKDTSKNQVVLTMTNMDPVDTATYYCAHRTGIAVAGIAFDIWGQGTMVTVSS

SEQ ID NO: 226 - protein, 107 aa, synthetic construct
QSVLTQPPSVSVSPGQTASITCSGDKLGDKYVSWYQQKPGQSPVLVIYQDTKRPSGIPERFSGSNSGNTATLTISGTQTVDEADYYCQAWDSSSLYVFGTGTKVTVL

SEQ ID NO: 227 - protein, 123 aa, synthetic construct
QLQLQESGPGLVKPSETLSLTCTVSGGSISGSSYYWVWIRQPPGKGLEWIGNIYSSGSTYYNPSFKSRVIISVDTSKSQFSLKLNSVTAADTAVYYCAKPLLRDYYFYGVDVWGQGTTVTVSS

SEQ ID NO: 228 - protein, 107 aa, synthetic construct
DIQLTPSPSFLSASVGDRVTITCRASQAISSYLAWYQQKPGKAPKLLISPASTLQSGVPSRFSGSGSGTEFTLTISSLQPEDFATYYCQQLKSYPWTFGQGTKVEIK

SEQ ID NO: 229 - protein, 124 aa, synthetic construct
EVQLLESGGGLVQPGGSLRLSCTASGFTFSSYAMSWVRQAPGKGLEWVSLISESGSSTYYADSVKGRFTNSRDDSRNTLYLQMNSLRAEDTAVYYCAKDRWFEGYHSYYGMDVWGQGTTVTVSS

SEQ ID NO: 230 - protein, 108 aa, synthetic construct
DIQLTQSPSFLSAFVGDRVTITCRASQGISNNLAWYQQKPGKAPKLLVYATSTLQSGVPSRFSGSGSGTEFTLTVSSLQPEDFATYYCQQLKSYPSFTFGPGTKVDIK

SEQ ID NO: 231 - protein, 123 aa, synthetic construct
QLQLQESGPGLVKPSETLSLTCTVSGGSISSSSYYWVWIRQSPGKGLEWIGNIYSSGSTHYNPSLKSRVIISVDTSKNQFSLKLKSVTAADTAVYYCAKPLLRDYYYYGVDVWGQGTTVTVSS

SEQ ID NO: 232 - protein, 107 aa, synthetic construct
DIQLTPSPSFLSASVGDRVTITCRASQAISSYLAWYQQKPGKAPKLLISPASTLQSGVPSRFSGSGSGTEFTLTISSLQPEDFATYFCQQLNSFPWTFGQGTKVEIK

SEQ ID NO: 233 - protein, 121 aa, synthetic construct
QVQLQQSGTEVKKPGASVKVSCKASGYTFTTYGLTWIRQAPGQGLEWMGWISVFNGSPNYSLKFQGRVTMTADTFTNTAYMELRSLRSDDTAVYYCARGGTGDYYPLFDYWGQGTLVTVSS

SEQ ID NO: 234 - protein, 107 aa, synthetic construct
AIRMTQSPSSLSASVGDRVTITCRASQGIRNDLGWYQQKPGKAPKLLIFAASNLESGVPSRFSGSRFGTDFTLTISSLQPEDFATYYCLQDYNYPYTFGQGTKLEIK

SEQ ID NO: 235 - protein, 121 aa, synthetic construct
QVQLLESGPGLVKPAETLSLTCTVSGGSITSTSYFWDWIRQPPGKGLEWIGSIYHSGSTYYDSSLKTRVTISVDTSKYQFSLRLSSVTAADTAVYYCARHEFGIRDAFDIWGQGTLVTVSS

SEQ ID NO: 236 - protein, 107 aa, synthetic construct
EIVMTQSPDFQSVTPKEKVTITCRASQSIGNNLHWYQQKPDQSPKLLIKYASQSISGVPSRFSGSGFGTDFTLTINSLEAEDAAAYYCHQSNSFPFTFGPGTKVEIK

SEQ ID NO: 237 - protein, 124 aa, synthetic construct
EVQLLESGGGLVQPGGSLRLSCTASGFTFSSYAMSWVREAPGKGLEWVSLISESGSSTYYAESVKGRFTISRDDSKNTLYLQMNSLRAEDTAVYYCAKDRWFEGYHSYYGMDVWGQGTTVTVSS

SEQ ID NO: 238 - protein, 108 aa, synthetic construct
DIQLTQSPSFLSAFVGDRVTITCRASQGISNNLAWYQKKPGKAPKLLVYATSTLQSGVPSRFSGSGSGTEFTLTVSSLQPEDFATYYCQQLKSYPSFTFGPGTKVDIK

SEQ ID NO: 239 - protein, 124 aa, synthetic construct
EVQLLESGGGLVQPGGSLRLSCTASGFTFSSYAMSWVREAPGKGLEWVSLISESGSSTYYAESVKGRFTNSRDDSRNTLYLQMNSLRAEDTAVYYCAKDRWFEGYHSYYGMDVWGQGTTVTVSS

SEQ ID NO: 240 - protein, 123 aa, synthetic construct
QLQLQESGPGLVKPSETLSLTCTVSGGSISSSSYYWVWIRESPGKGLEWIGNIYSSGSTHYNPSLKSRVIISVGTSKNQFSLKLKSVTAADTAVYYCAKPLLRDYYYYGVDVWGQGTTVTVSS

SEQ ID NO: 241 - protein, 107 aa, synthetic construct
DIQLTPSPSFLSASVGDRVTITCRASQAISSYLAWYQKKPGKAPKLLISPASTLQSGVPSRFSGSGSGTEFTLTISSLQPEDFATYFCQQLNSFPWTFGQGTKVEIK

SEQ ID NO: 242 - protein, 341 aa, synthetic construct; identical to SEQ ID NO: 138

SEQ ID NO: 243 - protein, 579 aa, synthetic construct; identical to SEQ ID NO: 139

SEQ ID NO: 244 - protein, 452 aa, synthetic construct
QLQLQESGPGLVKPSETLSLTCTVSGGSISSSSYYWVWIRESPGKGLEWIGNIYSSGSTHYNPSLKSRVIISVGTSKNQFSLKLKSVTAADTAVYYCAKPLLRDYYYYGVDVWGQGTTVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDEKVEPDSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 245 - protein, 214 aa, synthetic construct
DIQLTPSPSFLSASVGDRVTITCRASQAISSYLAWYQKKPGKAPKLLISPASTLQSGVPSRFSGSGSGTEFTLTISSLQPEDFATYFCQQLNSFPWTFGQGTKVEIKRTVAAPAVFIFPPSKKQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 246 - protein, 341 aa, synthetic construct; identical to SEQ ID NO: 138

SEQ ID NO: 247 - protein, 579 aa, synthetic construct; identical to SEQ ID NO: 143

SEQ ID NO: 248 - protein, 453 aa, synthetic construct
EVQLLESGGGLVQPGGSLRLSCTASGFTFSSYAMSWVREAPGKGLEWVSLISESGSSTYYAESVKGRFTISRDDSKNTLYLQMNSLRAEDTAVYYCAKDRWFEGYHSYYGMDVWGQGTTVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDEKVEPDSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 249 - protein, 215 aa, synthetic construct
DIQLTQSPSFLSAFVGDRVTITCRASQGISNNLAWYQKKPGKAPKLLVYATSTLQSGVPSRFSGSGSGTEFTLTVSSLQPEDFATYYCQQLKSYPSFTFGPGTKVDIKRTVAAPAVFIFPPSKKQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 250 - protein, 341 aa, synthetic construct; identical to SEQ ID NO: 138

SEQ ID NO: 251 - protein, 579 aa, synthetic construct; identical to SEQ ID NO: 139

SEQ ID NO: 252 - protein, 453 aa, synthetic construct
EVQLLESGGGLVQPGGSLRLSCTASGFTFSSYAMSWVREAPGKGLEWVSLISESGSSTYYAESVKGRFTNSRDDSRNTLYLQMNSLRAEDTAVYYCAKDRWFEGYHSYYGMDVWGQGTTVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDEKVEPDSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 253 - protein, 215 aa, synthetic construct; identical to SEQ ID NO: 249

SEQ ID NO: 254 - protein, 216 aa, synthetic construct
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 255 - protein, 124 aa, synthetic construct
TESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRT

SEQ ID NO: 256 - protein, 237 aa, synthetic construct
QLQLQESGPGLVKPSETLSLTCTVSGGSISGSSYYWVWIRQPPGKGLEWIGNIYSSGSTYYNPSFKSRVIISVDTSKSQFSLKLNSVTAADTAVYYCAKPLLRDYYFYGVDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTHHHHHH

SEQ ID NO: 257 - protein, 214 aa, synthetic construct
DIQLTPSPSFLSASVGDRVTITCRASQAISSYLAWYQQKPGKAPKLLISPASTLQSGVPSRFSGSGSGTEFTLTISSLQPEDFATYYCQQLKSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 258 - protein, 237 aa, synthetic construct
QLQLQESGPGLVKPSETLSLTCTVSGGSISSSSYYWVWIRQSPGKGLEWIGNIYSSGSTHYNPSLKSRVIISVDTSKNQFSLKLKSVTAADTAVYYCAKPLLRDYYYYGVDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTHHHHHH

SEQ ID NO: 259 - protein, 214 aa, synthetic construct
DIQLTPSPSFLSASVGDRVTITCRASQAISSYLAWYQQKPGKAPKLLISPASTLQSGVPSRFSGSGSGTEFTLTISSLQPEDFATYFCQQLNSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 260 - protein, 238 aa, synthetic construct
EVQLLESGGGLVQPGGSLRLSCTASGFTFSSYAMSWVRQAPGKGLEWVSLISESGSSTYYADSVKGRFTNSRDDSRNTLYLQMNSLRAEDTAVYYCAKDRWFEGYHSYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTHHHHHH

SEQ ID NO: 261 - protein, 215 aa, synthetic construct
DIQLTQSPSFLSAFVGDRVTITCRASQGISNNLAWYQQKPGKAPKLLVYATSTLQSGVPSRFSGSGSGTEFTLTVSSLQPEDFATYYCQQLKSYPSFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 262 - protein, 21 aa, synthetic construct
GLEWIGEIFHIGGTIYNPSLK

SEQ ID NO: 263 - protein, 10 aa, synthetic construct
ITCSGDALPK

SEQ ID NO: 264 - protein, 11 aa, synthetic construct
NTLYLQMNSLR

SEQ ID NO: 265 - protein, 14 aa, synthetic construct
SGTASVVCCAMLLK

SEQ ID NO: 266 - protein, 17 aa, synthetic construct
TTPPVLDSDGSFFLVSK

SEQ ID NO: 267 - protein, 17 aa, synthetic construct
TTPPVLDSDGSFFLYSK

SEQ ID NO: 268 - protein, 14 aa, synthetic construct
TVAAPAVFIFPPSK

SEQ ID NO: 269 - protein, 9 aa, synthetic construct
VTISVDTSK

SEQ ID NO: 270 - protein, 17 aa, synthetic construct
VYACEVTHQGLSSPVTK

SEQ ID NO: 271 - protein, 21 aa, synthetic construct
GLEWIGEINHIGGTIYNPSLK

SEQ ID NO: 272 - protein, 12 aa, synthetic construct
GPSVFPLAPSSK

SEQ ID NO: 273 - protein, 8 aa, synthetic construct
NDLGWYQK

SEQ ID NO: 274 - protein, 19 aa, synthetic construct
SYELTQPPSVSVSPGQTAR

SEQ ID NO: 275 - protein, 11 aa, synthetic construct
DSERPSGIPER

SEQ ID NO: 276 - protein, 16 aa, synthetic construct
LLIYDASNLETGVPSR

SEQ ID NO: 277 - protein, 19 aa, synthetic construct
SGTASVVCCAMLLNNFYPR

SEQ ID NO: 278 - protein, 18 aa, synthetic construct
TVAAPSVFIFPPSDEQLK

SEQ ID NO: 279 - protein, 20 aa, synthetic construct
VYACCAMEVTHQGLSSPVTK

SEQ ID NO: 280 - protein, 19 aa, synthetic construct
LSSVTAADTAVYYCCAMAR

SEQ ID NO: 281 - protein, 12 aa, synthetic construct
IEFPYTFGQGTK

SEQ ID NO: 282 - protein, 16 aa, synthetic construct
KPGQSPQLLIYTFFYR

SEQ ID NO: 283 - protein, 22 aa, synthetic construct
GFYPSDIAVEWESNGQPENNYK

SEQ ID NO: 284 - protein, 16 aa, synthetic construct
FLIFGTYSLQSGVPSR

(19) United States
(12) Patent Application Publication
(10) Pub. No.: US 2026/0242461 A1
(43) Pub. Date: Aug. 20, 2026

(54) TRISPECIFIC BINDING PROTEINS THAT BIND INTERLEUKIN-4 AND INTERLEUKIN-13

(71) Applicant: Sanofi, Paris (FR)

(72) Inventors: Christian BEIL, Frankfurt am Main (DE); Virginia BROPHY, Cambridge, MA (US); Béatrice CAMERON, Paris (FR); Klervi DESRUMEAUX, Paris (FR); Brian HALL, Cambridge, MA (US); Sagar KATHURIA, Cambridge, MA (US); Jennifer KüHN, Frankfurt am Main (DE); Brian MACKNESS, Cambridge, MA (US); Maureen MAGNAY, Cambridge, MA (US); Alessandro MASIERO, Paris (FR); Nels P. NIELSON, Lebanon, NH (US); Yu QIU, Cambridge, MA (US); Anusuya RAMASUBRAMANIAN, Cambridge, MA (US); Sambasiva RAO, Cambridge, MA (US); Pierrick RIVAL, Paris (FR); Emmanuelle VIGNE, Paris (FR); Yui-Hsi WANG, Cambridge, MA (US); Xiaocong YU, Cambridge, MA (US)

(21) Appl. No.: 19/543,590

(22) Filed: Feb. 18, 2026

(30) Foreign Application Priority Data
Feb. 19, 2025 (EP) 25305222.9
Feb. 19, 2025 (EP) 25305223.7
Jan. 13, 2026 (EP) 26305048.6
Feb. 13, 2026 (TW) 115106204
Feb. 13, 2026 (TW) 115106215
Feb. 13, 2026 (TW) 115106238

(51) Int. Cl. C07K 16/24; A61K 39/00; C07K 16/28

(52) U.S. Cl. CPC C07K 16/247; C07K 16/244; C07K 16/2851; C07K 16/2875; A61K 2039/505; C07K 2317/31; C07K 2317/92; C07K 2317/94

(57) ABSTRACT

Provided herein are trispecific binding proteins comprising four polypeptide chains that form three antigen binding sites that specifically bind one or more target proteins, wherein a first pair of polypeptides forming the binding protein possess dual variable domains having a cross-over orientation, and at least two of the antigen binding sites specifically bind an interleukin-4 (IL-4) polypeptide and an interleukin-13 (IL-13) polypeptide, respectively. In some embodiments, the third antigen binding site specifically binds an OX40L polypeptide, an interleukin-17 (IL-17) polypeptide, or a natural killer group 2D (NKG2D) polypeptide. The disclosure also relates to nucleic acid molecules, expression vectors, host cells, compositions (e.g., pharmaceutical compositions), methods of production, methods of use, and uses related thereto.

### CROSS-REFERENCE TO RELATED APPLICATIONS

This application claims the priority benefit of European Patent Application Nos. EP25305222.9, filed Feb. 19, 2025; EP25305223.7, filed Feb. 19, 2025; and EP26305048.6, filed Jan. 13, 2026; and Taiwanese Patent Application Nos. 115106215, filed Feb. 13, 2026; 115106238, filed Feb. 13, 2026; and 115106204, filed Feb. 13, 2026; each of which is incorporated herein by reference in its entirety.

### REFERENCE TO AN ELECTRONIC SEQUENCE LISTING

The content of the electronic sequence listing (183952036600SEQLIST.xml; Size: 259,595 bytes; and Date of Creation: Feb. 13, 2026) is incorporated herein by reference in its entirety.

### FIELD

The disclosure relates to trispecific binding proteins comprising four polypeptide chains that form three antigen binding sites that specifically bind one or more target proteins, wherein a first pair of polypeptides forming the binding protein possess dual variable domains having a cross-over orientation, and at least two of the antigen binding sites specifically bind an interleukin-4 (IL-4) polypeptide and an interleukin-13 (IL-13) polypeptide, respectively. The disclosure also relates to nucleic acid molecules, expression vectors, host cells, compositions (e.g., pharmaceutical compositions), methods of production, methods of use, and uses related thereto.

### BACKGROUND

Monoclonal antibody based biotherapeutics have become an important avenue for new drug development. Monoclonal antibody technology offers specific targeting, precise signaling delivery and/or payload to specific cell population, and provides long lasting biological effect through its Fc functions. Efforts in antibody engineering have allowed developing bispecific antibodies combining the specificities of two monoclonal antibodies for various biological applications, expanding the scope of antibody drug development. Newly discovered neutralizing antibodies with improved breadth and potency may provide more options for developing biotherapeutics to treat complexed diseases such as inflammatory diseases or disorders.

IL-4 and IL-13 are T helper 2 (Th2) cytokines that play important roles in inflammation (e.g., type II inflammatory responses). IL-17 is a family of six cytokines (IL-17A, B, C, D, E, and F) acting via five receptors (IL-17RA, B, C, D, and E). IL-17 cytokines are secreted by Th17 cells, as well as other cells of the immune system, and play roles in the secretion of pro-inflammatory cytokines, neutrophil-specific chemokines, and anti-microbial peptides. Monoclonal antibody drugs targeting IL-17, which include secukinumab, ixekizumab, bimekizumab, and brodalumab, are approved for the treatment of diseases such as plaque psoriasis, psoriatic arthritis, axial spondyloarthritis, and ankylosing spondylitis. See, e.g., Tiburca, L. et al. (2022) Curr Issues Mol Biol. 44(5):1851-1866. Natural killer group 2D (NKG2D; encoded by Kirkl) is an activating receptor expressed by lymphocytes, and its engagement stimulates pro-inflammatory cytokines release and directs immune-meditated tissues cytotoxicity.

Thus, there is a need for effective new molecules targeting these molecules, e.g., for the treatment of immune/inflammatory diseases or disorders.

All references cited herein, including patent applications, patent publications, and UniProtKB/Swiss-Prot Accession numbers are herein incorporated by reference in their entirety, as if each individual reference were specifically and individually indicated to be incorporated by reference.

### BRIEF SUMMARY

To meet these and other needs, provided herein are binding proteins (e.g., antibodies) that specifically bind IL-4 and/or IL-13. These binding proteins can specifically bind one, two, or three antigen targets or target proteins, such as IL-4, IL-13, and other target proteins such as OX40L, IL-17, or NKG2D. The anti-IL-4 and anti-IL-13 antigen binding sites (as well as binding proteins comprising the same) described herein have advantageous properties such as high affinity binding to human and cynomolgus IL-4 and IL-13 polypeptides, good manufacturability, strong inhibition of IL-4 and IL-13 signaling, favorable pharmacokinetics, good stability, and low propensity for aggregation/opalescence/viscosity. These antigen binding sites are contemplated for use in a variety of monospecific, bispecific, trispecific, and multispecific binding proteins.

Exemplary anti-IL-17 antigen binding sites described herein were also found to display similar binding affinity to monovalent comparator molecules when tested in a trispecific CODV format. Such antigen binding sites are contemplated for use in a variety of monospecific, bispecific, trispecific, and multispecific binding proteins. For instance, the instant application demonstrates that trispecific anti-IL-4/IL-13/IL-17 binding proteins were able to bind all three targets with similar affinity as compared to monospecific and bispecific comparator molecules, were stable, and were able to inhibit IL-4, IL-13, and IL-17 activities in vivo.

Without wishing to be bound to theory, it is thought that neutralizing IL-4 and IL-13 can reduce type 2 inflammation, and combining this activity with neutralization of certain non-type 2 inflammation (e.g., NK cell activities) can simultaneously target both of these inflammatory pathways. The anti-NKG2D antigen binding sites described herein were found to bind human and cynomolgus NKG2D polypeptides with high affinity and block NKG2D ligands, MICA, MICB, and ULBP6 binding, and trispecific binding proteins that bind IL-4, IL-13 and NKG2D were found to bind all three targets with high affinity and possess beneficial manufacturability and purification properties.

In some embodiments, provided herein are trispecific and/or trivalent binding proteins comprising four polypeptide chains that form three antigen binding sites, wherein a first polypeptide chain of the binding protein comprises a structure represented by the formula:

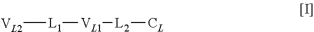

and a second polypeptide chain of the binding protein comprises a structure represented by the formula:

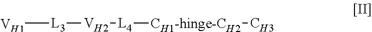

and a third polypeptide chain of the binding protein comprises a structure represented by the formula:

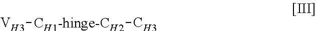

and a fourth polypeptide chain of the binding protein comprises a structure represented by the formula:

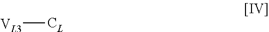

wherein:
   V_{L1} is a first immunoglobulin light chain variable domain;
   V_{L2} is a second immunoglobulin light chain variable domain;
   V_{L3} is a third immunoglobulin light chain variable domain;
   V_{H1} is a first immunoglobulin heavy chain variable domain;
   V_{H2} is a second immunoglobulin heavy chain variable domain;
   V_{H3} is a third immunoglobulin heavy chain variable domain;
   C_{L} is an immunoglobulin light chain constant domain;
   C_{H1} is an immunoglobulin C_{H1} heavy chain constant domain;
   C_{H2} is an immunoglobulin C_{H2} heavy chain constant domain;
   C_{H3} is an immunoglobulin C_{H3} heavy chain constant domain;
   hinge is an immunoglobulin hinge region connecting the C_{H1} and C_{H2} domains; and
   L₁, L₂, L₃ and L₄ are amino acid linkers;
wherein the polypeptide of formula I and the polypeptide of formula II form a cross-over light chain-heavy chain pair; and
wherein V_{H1} and V_{L1} form a binding pair and a first antigen binding site, V_{H2} and V_{L2} form a binding pair and a second antigen binding site, and V_{H3} and V_{L3} form a binding pair and a third antigen binding site. In some embodiments, the first antigen binding site specifically binds an interleukin-4 (IL-4) polypeptide and the second antigen binding site specifically binds an interleukin-13 (IL-13) polypeptide. In some embodiments, the first antigen binding site specifically binds an IL-13 polypeptide and the second antigen binding site specifically binds an IL-4 polypeptide. In some embodiments, the binding site that specifically binds an IL-4 polypeptide comprises a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence selected from the group consisting of SEQ ID NOs:3-5, and a VL domain that comprises a CDR-L1 comprising the amino acid sequence of SEQ ID NO:6 or 7, a CDR-L2 comprising the amino acid sequence of SEQ ID NO:8 or 9, and a CDR-L3 comprising the amino acid sequence of SEQ ID NO:10 or 11. In some embodiments, the binding site that specifically binds an IL-4 polypeptide comprises a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence selected from the group consisting of SEQ ID NOs:24-27, and a VL domain that comprises a CDR-L₁ comprising the amino acid sequence of SEQ ID NO:6 or 7, a CDR-L2 comprising the amino acid sequence of SEQ ID NO:8 or 9, and a CDR-L3 comprising the amino acid sequence of SEQ ID NO:10 or 11. In some embodiments, the binding site that specifically binds an IL-13 polypeptide comprises a VH domain that comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59 or 108, and a VL domain that comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62. In some embodiments, L₁, L₂, L₃ and L₄ are each independently amino acid linkers or zero amino acids in length.

In some embodiments, provided herein are bispecific binding proteins comprising a first antigen binding site specifically binds an interleukin-4 (IL-4) polypeptide and a second antigen binding site specifically binds an interleukin-13 (IL-13) polypeptide. In some embodiments, the bispecific binding proteins comprise three polypeptide chains that form two antigen binding sites, wherein a first polypeptide chain of the binding protein comprises a structure represented by the formula:

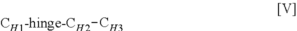

and a second polypeptide chain of the binding protein comprises a structure represented by the formula:

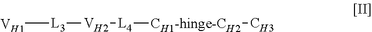

and a third polypeptide chain of the binding protein comprises a structure represented by the formula:

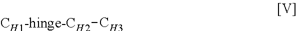

wherein:
   V_{L1} is a first immunoglobulin light chain variable domain;
   V_{L2} is a second immunoglobulin light chain variable domain;
   V_{L3} is a third immunoglobulin light chain variable domain;
   V_{H1} is a first immunoglobulin heavy chain variable domain;
   V_{H2} is a second immunoglobulin heavy chain variable domain;
   V_{H3} is a third immunoglobulin heavy chain variable domain;
   C_{L} is an immunoglobulin light chain constant domain;
   C_{H1} is an immunoglobulin C_{H1} heavy chain constant domain;
   C_{H2} is an immunoglobulin C_{H2} heavy chain constant domain;
   C_{H3} is an immunoglobulin C_{H3} heavy chain constant domain;
   hinge is an immunoglobulin hinge region connecting the C_{H1} and C_{H2} domains; and
   L₁, L₂, L₃ and L₄ are amino acid linkers;
wherein the polypeptide of formula I and the polypeptide of formula II form a cross-over light chain-heavy chain pair; and wherein V_{H1} and V_{L1} form a binding pair and a first antigen binding site, and V_{H2} and V_{L2} form a binding pair and a second antigen binding site. In some embodiments, the first antigen binding site specifically binds an interleukin-4 (IL-4) polypeptide and the second antigen binding site specifically binds an interleukin-13 (IL-13) polypeptide. In some embodiments, the first antigen binding site specifically binds an IL-13 polypeptide and the second antigen binding site specifically binds an IL-4 polypeptide. In some embodiments, the binding site that specifically binds an IL-4 polypeptide comprises a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence selected from the group consisting of SEQ ID NOs:3-5, and a VL domain that comprises a CDR-L1 comprising the amino acid sequence of SEQ ID NO:6 or 7, a CDR-L2 comprising the amino acid sequence of SEQ ID NO:8 or 9, and a CDR-L3 comprising the amino acid sequence of SEQ ID NO:10 or 11. In some embodiments, the binding site that specifically binds an IL-4 polypeptide comprises a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence selected from the group consisting of SEQ ID NOs:24-27, and a VL domain that comprises a CDR-L1 comprising the amino acid sequence of SEQ ID NO:6 or 7, a CDR-L2 comprising the amino acid sequence of SEQ ID NO:8 or 9, and a CDR-L3 comprising the amino acid sequence of SEQ ID NO:10 or 11. In some embodiments, the binding site that specifically binds an IL-13 polypeptide comprises a VH domain that comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59 or 108, and a VL domain that comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62. In some embodiments, L₁, L₂, L₃ and L₄ are each independently amino acid linkers or zero amino acids in length.

In some embodiments according to any one of the embodiments disclosed herein, the binding site that specifically binds an IL-4 polypeptide comprises a VH domain that comprises a CDR-H1 comprising the amino acid sequence of SEQ ID NO: 1, a CDR-H2 comprising the amino acid sequence of SEQ ID NO:2, and a CDR-H3 comprising the amino acid sequence of SEQ ID NO:3, and a VL domain that comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10. In some embodiments, the binding site that specifically binds an IL-4 polypeptide comprises a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3, and a VL domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11. In some embodiments, the binding site that specifically binds an IL-4 polypeptide comprises a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:4, and a VL domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11. In some embodiments, the binding site that specifically binds an IL-4 polypeptide comprises a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:5, and a VL domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:34 and a VL domain that comprises an amino acid sequence that is at least 95%, at least 96%, at least 97%, at least 98%, at least 99%, or 100% identical to the amino acid sequence of SEQ ID NO:35. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:36 and a VL domain that comprises an amino acid sequence that is at least 95%, at least 96%, at least 97%, at least 98%, at least 99%, or 100% identical to the amino acid sequence of SEQ ID NO:37. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:38 and a VL domain that comprises an amino acid sequence that is at least 95%, at least 96%, at least 97%, at least 98%, at least 99%, or 100% identical to the amino acid sequence of SEQ ID NO:39. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:40 and a VL domain that comprises an amino acid sequence that is at least 95%, at least 96%, at least 97%, at least 98%, at least 99%, or 100% identical to the amino acid sequence of SEQ ID NO:41. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:42 and a VL domain that comprises an amino acid sequence that is at least 95%, at least 96%, at least 97%, at least 98%, at least 99%, or 100% identical to the amino acid sequence of SEQ ID NO:43. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:44 and a VL domain that comprises an amino acid sequence that is at least 95%, at least 96%, at least 97%, at least 98%, at least 99%, or 100% identical to the amino acid sequence of SEQ ID NO:43. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:45 and a VL domain that comprises an amino acid sequence that is at least 95%, at least 96%, at least 97%, at least 98%, at least 99%, or 100% identical to the amino acid sequence of SEQ ID NO:43. In some embodiments, V_{H1} comprises an amino acid sequence that is at least 95%, at least 96%, at least 97%, at least 98%, at least 99%, or 100% identical to the amino acid sequence of SEQ ID NO:34, and V_{L1} comprises an amino acid sequence that is at least 95%, at least 96%, at least 97%, at least 98%, at least 99%, or 100% identical to the amino acid sequence of SEQ ID NO:35. In some embodiments, V_{H1} comprises an amino acid sequence that is at least 95%, at least 96%, at least 97%, at least 98%, at least 99%, or 100% identical to the amino acid sequence of SEQ ID NO:36, and V_{L1} comprises an amino acid sequence that is at least 95%, at least 96%, at least 97%, at least 98%, at least 99%, or 100% identical to the amino acid sequence of SEQ ID NO:37. In some embodiments, V_{H1} comprises an amino acid sequence that is at least 95%, at least 96%, at least 97%, at least 98%, at least 99%, or 100% identical to the amino acid sequence of SEQ ID NO:38, and V_{L1} comprises an amino acid sequence that is at least 95%, at least 96%, at least 97%, at least 98%, at least 99%, or 100% identical to the amino acid sequence of SEQ ID NO:39. In some embodiments, V_{H1} comprises an amino acid sequence that is at least 95%, at least 96%, at least 97%, at least 98%, at least 99%, or 100% identical to the amino acid sequence of SEQ ID NO:40, and V_{L1} comprises an amino acid sequence that is at least 95%, at least 96%, at least 97%, at least 98%, at least 99%, or 100% identical to the amino acid sequence of SEQ ID NO:41. In some embodiments, V_{H1} comprises an amino acid sequence that is at least 95%, at least 96%, at least 97%, at least 98%, at least 99%, or 100% identical to the amino acid sequence of SEQ ID NO:42, and V_{L1} comprises an amino acid sequence that is at least 95%, at least 96%, at least 97%, at least 98%, at least 99%, or 100% identical to the amino acid sequence of SEQ ID NO:43. In some embodiments, V_{H1} comprises an amino acid sequence that is at least 95%, at least 96%, at least 97%, at least 98%, at least 99%, or 100% identical to the amino acid sequence of SEQ ID NO:44, and V_{L1} comprises an amino acid sequence that is at least 95%, at least 96%, at least 97%, at least 98%, at least 99%, or 100% identical to the amino acid sequence of SEQ ID NO:43. In some embodiments, V_{H1} comprises an amino acid sequence that is at least 95%, at least 96%, at least 97%, at least 98%, at least 99%, or 100% identical to the amino acid sequence of SEQ ID NO:45, and V_{L1} comprises an amino acid sequence that is at least 95%, at least 96%, at least 97%, at least 98%, at least 99%, or 100% identical to the amino acid sequence of SEQ ID NO:43. In some embodiments, the binding site that specifically binds an IL-4 polypeptide comprises a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24, and a VL domain that comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10. In some embodiments, the binding site that specifically binds an IL-4 polypeptide comprises a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24, and a VL domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11. In some embodiments, the binding site that specifically binds an IL-4 polypeptide comprises a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, and a VL domain that comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10. In some embodiments, the binding site that specifically binds an IL-4 polypeptide comprises a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, and a VL domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11. In some embodiments, the binding site that specifically binds an IL-4 polypeptide comprises a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:26, and a VL domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11. In some embodiments, the binding site that specifically binds an IL-4 polypeptide comprises a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:27, and a VL domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:46 and a VL domain that comprises an amino acid sequence that is at least 95%, at least 96%, at least 97%, at least 98%, at least 99%, or 100% identical to the amino acid sequence of SEQ ID NO:47. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:48 and a VL domain that comprises an amino acid sequence that is at least 95%, at least 96%, at least 97%, at least 98%, at least 99%, or 100% identical to the amino acid sequence of SEQ ID NO:39. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:49 and a VL domain that comprises an amino acid sequence that is at least 95%, at least 96%, at least 97%, at least 98%, at least 99%, or 100% identical to the amino acid sequence of SEQ ID NO:41. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:50 and a VL domain that comprises an amino acid sequence that is at least 95%, at least 96%, at least 97%, at least 98%, at least 99%, or 100% identical to the amino acid sequence of SEQ ID NO:43. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:51 and a VL domain that comprises an amino acid sequence that is at least 95%, at least 96%, at least 97%, at least 98%, at least 99%, or 100% identical to the amino acid sequence of SEQ ID NO:37. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:52 and a VL domain that comprises an amino acid sequence that is at least 95%, at least 96%, at least 97%, at least 98%, at least 99%, or 100% identical to the amino acid sequence of SEQ ID NO:39. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:53 and a VL domain that comprises an amino acid sequence that is at least 95%, at least 96%, at least 97%, at least 98%, at least 99%, or 100% identical to the amino acid sequence of SEQ ID NO:41. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:54 and a VL domain that comprises an amino acid sequence that is at least 95%, at least 96%, at least 97%, at least 98%, at least 99%, or 100% identical to the amino acid sequence of SEQ ID NO:43. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:55 and a VL domain that comprises an amino acid sequence that is at least 95%, at least 96%, at least 97%, at least 98%, at least 99%, or 100% identical to the amino acid sequence of SEQ ID NO:43. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:56 and a VL domain that comprises an amino acid sequence that is at least 95%, at least 96%, at least 97%, at least 98%, at least 99%, or 100% identical to the amino acid sequence of SEQ ID NO:43. In some embodiments, V_{H1} comprises an amino acid sequence that is at least 95%, at least 96%, at least 97%, at least 98%, at least 99%, or 100% identical to the amino acid sequence of SEQ ID NO:46, and V_{L1} comprises an amino acid sequence that is at least 95%, at least 96%, at least 97%, at least 98%, at least 99%, or 100% identical to the amino acid sequence of SEQ ID NO:47. In some embodiments, V_{H1} comprises an amino acid sequence that is at least 95%, at least 96%, at least 97%, at least 98%, at least 99%, or 100% identical to the amino acid sequence of SEQ ID NO:48, and V_{L1} comprises an amino acid sequence that is at least 95%, at least 96%, at least 97%, at least 98%, at least 99%, or 100% identical to the amino acid sequence of SEQ ID NO:39. In some embodiments, V_{H1} comprises an amino acid sequence that is at least 95%, at least 96%, at least 97%, at least 98%, at least 99%, or 100% identical to the amino acid sequence of SEQ ID NO:49, and V_{L1} comprises an amino acid sequence that is at least 95%, at least 96%, at least 97%, at least 98%, at least 99%, or 100% identical to the amino acid sequence of SEQ ID NO:41. In some embodiments, V_{H1} comprises an amino acid sequence that is at least 95%, at least 96%, at least 97%, at least 98%, at least 99%, or 100% identical to the amino acid sequence of SEQ ID NO:50, and V_{L1} comprises an amino acid sequence that is at least 95%, at least 96%, at least 97%, at least 98%, at least 99%, or 100% identical to the amino acid sequence of SEQ ID NO:43. In some embodiments, V_{H1} comprises an amino acid sequence that is at least 95%, at least 96%, at least 97%, at least 98%, at least 99%, or 100% identical to the amino acid sequence of SEQ ID NO:51, and V_{L1} comprises an amino acid sequence that is at least 95%, at least 96%, at least 97%, at least 98%, at least 99%, or 100% identical to the amino acid sequence of SEQ ID NO:37. In some embodiments, V_{H1} comprises an amino acid sequence that is at least 95%, at least 96%, at least 97%, at least 98%, at least 99%, or 100% identical to the amino acid sequence of SEQ ID NO:52, and V_{L1} comprises an amino acid sequence that is at least 95%, at least 96%, at least 97%, at least 98%, at least 99%, or 100% identical to the amino acid sequence of SEQ ID NO:39. In some embodiments, V_{H1} comprises an amino acid sequence that is at least 95%, at least 96%, at least 97%, at least 98%, at least 99%, or 100% identical to the amino acid sequence of SEQ ID NO:53, and V_{L1} comprises an amino acid sequence that is at least 95%, at least 96%, at least 97%, at least 98%, at least 99%, or 100% identical to the amino acid sequence of SEQ ID NO:41. In some embodiments, V_{H1} comprises an amino acid sequence that is at least 95%, at least 96%, at least 97%, at least 98%, at least 99%, or 100% identical to the amino acid sequence of SEQ ID NO:54, and V_{L1} comprises an amino acid sequence that is at least 95%, at least 96%, at least 97%, at least 98%, at least 99%, or 100% identical to the amino acid sequence of SEQ ID NO:43. In some embodiments, V_{H1} comprises an amino acid sequence that is at least 95%, at least 96%, at least 97%, at least 98%, at least 99%, or 100% identical to the amino acid sequence of SEQ ID NO:55, and V_{L1} comprises an amino acid sequence that is at least 95%, at least 96%, at least 97%, at least 98%, at least 99%, or 100% identical to the amino acid sequence of SEQ ID NO:43. In some embodiments, V_{H1} comprises an amino acid sequence that is at least 95%, at least 96%, at least 97%, at least 98%, at least 99%, or 100% identical to the amino acid sequence of SEQ ID NO:56, and V_{L1} comprises an amino acid sequence that is at least 95%, at least 96%, at least 97%, at least 98%, at least 99%, or 100% identical to the amino acid sequence of SEQ ID NO:43.

In some embodiments, the binding site that specifically binds an IL-13 polypeptide comprises a VH domain that comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, and a VL domain that comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62. In some embodiments, the binding site that specifically binds an IL-13 polypeptide comprises a VH domain that comprises an amino acid sequence that is at least 95%, at least 96%, at least 97%, at least 98%, at least 99%, or 100% identical to the amino acid sequence of SEQ ID NO:70 and a VL domain that comprises an amino acid sequence that is at least 95%, at least 96%, at least 97%, at least 98%, at least 99%, or 100% identical to the amino acid sequence of SEQ ID NO:71. In some embodiments, the binding site that specifically binds an IL-13 polypeptide comprises a VH domain that comprises an amino acid sequence that is at least 95%, at least 96%, at least 97%, at least 98%, at least 99%, or 100% identical to the amino acid sequence of SEQ ID NO:72 and a VL domain that comprises an amino acid sequence that is at least 95%, at least 96%, at least 97%, at least 98%, at least 99%, or 100% identical to the amino acid sequence of SEQ ID NO:73. In some embodiments, the binding site that specifically binds an IL-13 polypeptide comprises a VH domain that comprises an amino acid sequence that is at least 95%, at least 96%, at least 97%, at least 98%, at least 99%, or 100% identical to the amino acid sequence of SEQ ID NO:74 and a VL domain that comprises an amino acid sequence that is at least 95%, at least 96%, at least 97%, at least 98%, at least 99%, or 100% identical to the amino acid sequence of SEQ ID NO:75. In some embodiments, V_{H2} comprises an amino acid sequence that is at least 95%, at least 96%, at least 97%, at least 98%, at least 99%, or 100% identical to the amino acid sequence of SEQ ID NO:70, and V_{L2} comprises an amino acid sequence that is at least 95%, at least 96%, at least 97%, at least 98%, at least 99%, or 100% identical to the amino acid sequence of SEQ ID NO:71. In some embodiments, V_{H2} comprises an amino acid sequence that is at least 95%, at least 96%, at least 97%, at least 98%, at least 99%, or 100% identical to the amino acid sequence of SEQ ID NO:72, and V_{L2} comprises an amino acid sequence that is at least 95%, at least 96%, at least 97%, at least 98%, at least 99%, or 100% identical to the amino acid sequence of SEQ ID NO:73. In some embodiments, V_{H2} comprises an amino acid sequence that is at least 95%, at least 96%, at least 97%, at least 98%, at least 99%, or 100% identical to the amino acid sequence of SEQ ID NO:74, and V_{L2} comprises an amino acid sequence that is at least 95%, at least 96%, at least 97%, at least 98%, at least 99%, or 100% identical to the amino acid sequence of SEQ ID NO:75. In some embodiments, the binding site that specifically binds an IL-13 polypeptide comprises a VH domain that comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:108, and a VL domain that comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62. In some embodiments, the binding site that specifically binds an IL-13 polypeptide comprises a VH domain that comprises an amino acid sequence that is at least 95%, at least 96%, at least 97%, at least 98%, at least 99%, or 100% identical to the amino acid sequence of SEQ ID NO:76 and a VL domain that comprises an amino acid sequence that is at least 95%, at least 96%, at least 97%, at least 98%, at least 99%, or 100% identical to the amino acid sequence of SEQ ID NO:77. In some embodiments, V_{H2} comprises an amino acid sequence that is at least 95%, at least 96%, at least 97%, at least 98%, at least 99%, or 100% identical to the amino acid sequence of SEQ ID NO:76, and V_{L2} comprises an amino acid sequence that is at least 95%, at least 96%, at least 97%, at least 98%, at least 99%, or 100% identical to the amino acid sequence of SEQ ID NO:77.

In some embodiments according to any one of the embodiments disclosed herein, one or more (e.g., one, two, or all three) of the antigen binding sites comprise a pair of reciprocal amino acid substitutions on the VH and VL domains that facilitate heavy-light chain pairing.

In some embodiments according to any one of the embodiments disclosed herein, V_{H1} comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3; V_{L}, comprises a CDR-L1 comprising the amino acid sequence of SEQ ID NO: 6, a CDR-L2 comprising the amino acid sequence of SEQ ID NO:8, and a CDR-L3 comprising the amino acid sequence of SEQ ID NO:10; V_{H2} comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59; and V_{L2} comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62. In some embodiments, V_{H1} comprises the amino acid sequence of SEQ ID NO:34, V_{L}, comprises the amino acid sequence of SEQ ID NO:35, V_{H2} comprises the amino acid sequence of SEQ ID NO:70, and V_{L2} comprises the amino acid sequence of SEQ ID NO:71.

In some embodiments according to any one of the embodiments disclosed herein, the binding protein is capable of inhibiting the function of one or more target proteins. In some embodiments, the binding protein is capable of inhibiting the function of an IL-4 polypeptide, an IL-13 polypeptide, or both.

In some embodiments according to any one of the embodiments disclosed herein, the third antigen binding site specifically binds an OX40L polypeptide. In some embodiments, the binding protein is capable of inhibiting the function of an OX40L polypeptide. In some embodiments, the binding site that specifically binds an OX40L polypeptide comprises a VH domain that comprises a CDR-H1 comprising the amino acid sequence of SEQ ID NO:78, a CDR-H2 comprising the amino acid sequence of SEQ ID NO:79, and a CDR-H3 comprising the amino acid sequence of SEQ ID NO:80; and a VL domain that comprises a CDR-L1 comprising the amino acid sequence of SEQ ID NO:81, a CDR-L2 comprising the amino acid sequence of SEQ ID NO:82, and a CDR-L3 comprising the amino acid sequence of SEQ ID NO:83. In some embodiments, V_{H3} comprises a CDR-H1 comprising the amino acid sequence of SEQ ID NO:78, a CDR-H2 comprising the amino acid sequence of SEQ ID NO:79, and a CDR-H3 comprising the amino acid sequence of SEQ ID NO:80; and V_{L3} comprises a CDR-L1 comprising the amino acid sequence of SEQ ID NO: 81, a CDR-L2 comprising the amino acid sequence of SEQ ID NO:82, and a CDR-L3 comprising the amino acid sequence of SEQ ID NO:83. In some embodiments, the binding site that specifically binds an OX40L polypeptide comprises a VH domain that comprises an amino acid sequence that is at least 95%, at least 96%, at least 97%, at least 98%, at least 99%, or 100% identical to the amino acid sequence of SEQ ID NO:91, and a VL domain that comprises an amino acid sequence that is at least 95%, at least 96%, at least 97%, at least 98%, at least 99%, or 100% identical to the amino acid sequence of SEQ ID NO:92. In some embodiments, the binding site that specifically binds an OX40L polypeptide comprises a VH domain that comprises an amino acid sequence that is at least 95%, at least 96%, at least 97%, at least 98%, at least 99%, or 100% identical to the amino acid sequence of SEQ ID NO:93, and a VL domain that comprises an amino acid sequence that is at least 95%, at least 96%, at least 97%, at least 98%, at least 99%, or 100% identical to the amino acid sequence of SEQ ID NO:94. In some embodiments, the binding site that specifically binds an OX40L polypeptide comprises a VH domain that comprises an amino acid sequence that is at least 95%, at least 96%, at least 97%, at least 98%, at least 99%, or 100% identical to the amino acid sequence of SEQ ID NO:95, and a VL domain that comprises an amino acid sequence that is at least 95%, at least 96%, at least 97%, at least 98%, at least 99%, or 100% identical to the amino acid sequence of SEQ ID NO:96. In some embodiments, the binding site that specifically binds an OX40L polypeptide comprises a VH domain that comprises an amino acid sequence that is at least 95%, at least 96%, at least 97%, at least 98%, at least 99%, or 100% identical to the amino acid sequence of SEQ ID NO:97, and a VL domain that comprises an amino acid sequence that is at least 95%, at least 96%, at least 97%, at least 98%, at least 99%, or 100% identical to the amino acid sequence of SEQ ID NO:96. In some embodiments, V_{H3} comprises an amino acid sequence that is at least 95%, at least 96%, at least 97%, at least 98%, at least 99%, or 100% identical to the amino acid sequence of SEQ ID NO:91, and V_{L3} comprises an amino acid sequence that is at least 95%, at least 96%, at least 97%, at least 98%, at least 99%, or 100% identical to the amino acid sequence of SEQ ID NO:92. In some embodiments, V_{H3} comprises an amino acid sequence that is at least 95%, at least 96%, at least 97%, at least 98%, at least 99%, or 100% identical to the amino acid sequence of SEQ ID NO:93, and V_{L3} comprises an amino acid sequence that is at least 95%, at least 96%, at least 97%, at least 98%, at least 99%, or 100% identical to the amino acid sequence of SEQ ID NO:94. In some embodiments, V_{H3} comprises an amino acid sequence that is at least 95%, at least 96%, at least 97%, at least 98%, at least 99%, or 100% identical to the amino acid sequence of SEQ ID NO:95, and V_{L3} comprises an amino acid sequence that is at least 95%, at least 96%, at least 97%, at least 98%, at least 99%, or 100% identical to the amino acid sequence of SEQ ID NO:96. In some embodiments, V_{H3} comprises an amino acid sequence that is at least 95%, at least 96%, at least 97%, at least 98%, at least 99%, or 100% identical to the amino acid sequence of SEQ ID NO:97, and V_{L3} comprises an amino acid sequence that is at least 95%, at least 96%, at least 97%, at least 98%, at least 99%, or 100% identical to the amino acid sequence of SEQ ID NO:96. In some embodiments, the binding site that specifically binds an OX40L polypeptide binds to an epitope of human OX40L that at least partially overlaps with that of human OX40. In some embodiments, the binding site that specifically binds an OX40L polypeptide binds to an epitope of human OX40L comprising one or more of the following residues (numbering based on SEQ ID NO:110): Q65, T67, E68, T78, Q80, K106, G107, Y108, F109, S110, Q111, E112, D122, E123, E124, F127, Q128, L129, K130, K131, R133, S134, N136, D156, N157, T158, S159, L160, D161, D162, H164, and N166. In some embodiments, the binding site that specifically binds an OX40L polypeptide binds to an epitope of human OX40L comprising all of the following residues (numbering based on SEQ ID NO:110): Q65, T67, E68, T78, Q80, K106, G107, Y108, F109, 5110, Q111, E112, D122, E123, E124, F127, Q128, L129, K130, K131, R133, S134, N136, D156, N157, T158, S159, L160, D161, D162, H164, and N166. In some embodiments, the binding site that specifically binds an OX40L polypeptide binds to human OX40L with a paratope comprising one or more of the following VH domain residues: F27, T28, N30/S30, S31, Y32, D50, S52, S54, R56, S57, K98, D99, F102, C103, S104, A106, S107, C108, N109, E110, and Y113 (consecutive numbering of SEQ ID NO:97 as shown in FIG. 17, which can be converted to other numbering schemes like Kabat or IMGT) and/or one or more of the following VL domain residues: R30, N31, D32, S56, H91, S92, R93, Y94, P95, and W96 (consecutive numbering of SEQ ID NO: 96 as shown in FIG. 17, which can be converted to other numbering schemes like Kabat or IMGT). In some embodiments, the binding site that specifically binds an OX40L polypeptide binds to human OX40L with a paratope comprising one or more of the following VH domain residues: F27, T28, N30/530, S31, Y32, D50, S52, S54, R56, S57, K98, D99, F102, C103, S104, A106, S107, C108, N109, E110, and Y113 (consecutive numbering of SEQ ID NO:97) and one or more of the following VL domain residues: R30, N31, D32, S56, H91, S92, R93, Y94, P95, and W96 (consecutive numbering of SEQ ID NO:96). In some embodiments, the binding site that specifically binds an OX40L polypeptide binds to human OX40L with a paratope comprising all of the following VH domain residues: F27, T28, N30/530, S31, Y32, D50, S52, S54, R56, 557, K98, D99, F102, C103, 5104, A106, 5107, C108, N109, E110, and Y113 (consecutive numbering of SEQ ID NO:97) and all of the following VL domain residues: R30, N31, D32, S56, H91, S92, R93, Y94, P95, and W96 (consecutive numbering of SEQ ID NO:96).

In some embodiments according to any one of the embodiments disclosed herein, V_{H1} comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3; V_{L}, comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10; V_{H2} comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59; V_{L2} comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62; V_{H3} comprises a CDR-H1 comprising the amino acid sequence of SEQ ID NO:78, a CDR-H2 comprising the amino acid sequence of SEQ ID NO:79, and a CDR-H3 comprising the amino acid sequence of SEQ ID NO:80; and V_{L3} comprises a CDR-L1 comprising the amino acid sequence of SEQ ID NO:81, a CDR-L2 comprising the amino acid sequence of SEQ ID NO:82, and a CDR-L3 comprising the amino acid sequence of SEQ ID NO:83. In some embodiments, V_{H1} comprises the amino acid sequence of SEQ ID NO:34, V_{L}, comprises the amino acid sequence of SEQ ID NO:35, V_{H2} comprises the amino acid sequence of SEQ ID NO:70, V_{L2} comprises the amino acid sequence of SEQ ID NO:71, V_{H3} comprises the amino acid sequence of SEQ ID NO:91, and V_{L3} comprises the amino acid sequence of SEQ ID NO:92.

In some embodiments according to any one of the embodiments disclosed herein, at least one of L₁, L₂, L₃ or L₄, is independently 0 amino acids in length. In some embodiments, L₁, L₂, L₃ or L₄ are each independently at least one amino acid in length. In some embodiments, (a) L₁, L₂, L₃ and L₄ each independently are zero amino acids in length or comprise a sequence selected from the group consisting of GGGGSGGGGS (SEQ ID NO:102), GGGGSGGGGSGGGGS (SEQ ID NO:103), S, RT, TKGPS (SEQ ID NO:104), GQPKAAP (SEQ ID NO: 105), and GGSGSSGSGG (SEQ ID NO:106); or (b) L₁, L₂, L₃ and L₄ each independently comprise a sequence selected from the group consisting of GGGGSGGGGS (SEQ ID NO:102), GGGGSGGGGSGGGGS (SEQ ID NO:103), S, RT, TKGPS (SEQ ID NO: 104), GQPKAAP (SEQ ID NO: 105), and GGSGSSGSGG (SEQ ID NO: 106). In some embodiments, L₁ comprises the sequence GQPKAAP (SEQ ID NO: 105), L₂ comprises the sequence TKGPS (SEQ ID NO: 104), L3 comprises the sequence S, and L₄ comprises the sequence RT. In some embodiments, L₁ comprises the sequence GGGGSGGGGS (SEQ ID NO:102), L₂ comprises the sequence GGGGSGGGGS (SEQ ID NO: 102), L₃ is 0 amino acids in length, and L₄ is 0 amino acids in length. In some embodiments, L₁ comprises the sequence GGSGSSGSGG (SEQ ID NO:106), L₂ comprises the sequence GGSGSSGSGG (SEQ ID NO: 106), L₃ is 0 amino acids in length, and L₄ is 0 amino acids in length. In some embodiments, L₁ comprises the sequence GGGGSGGGGSGGGGS (SEQ ID NO:103), L₂ is 0 amino acids in length, L3 comprises the sequence GGGGSGGGGSGGGGS (SEQ ID NO:103), and L₄ is 0 amino acids in length.

In some embodiments according to any one of the embodiments disclosed herein, the C_{H3} domain of the second polypeptide chain comprises amino acid substitutions at positions corresponding to positions 354 and 366 of human IgG1 or IgG4 according to EU Index, wherein the amino acid substitutions are S354C and T366W; and wherein the C_{H3} domain of the third polypeptide chain comprises amino acid substitutions at positions corresponding to positions 349, 366, 368, and 407 of human IgG1 or IgG4 according to EU Index, wherein the amino acid substitutions are Y349C, T366S, L368A, and Y407V. In some embodiments, the C_{H3} domain of the second polypeptide chain comprises amino acid substitutions at positions corresponding to positions 349, 366, 368, and 407 of human IgG1 or IgG4 according to EU Index, wherein the amino acid substitutions are Y349C, T366S, L368A, and Y407V; and wherein the C_{H3} domain of the third polypeptide chain comprises amino acid substitutions at positions corresponding to positions 354 and 366 of human IgG1 or IgG4 according to EU Index, wherein the amino acid substitutions are S354C and T366W. In some embodiments, the C_{H3} domains of the second and the third polypeptide chains are human IgG1 or IgG4 C_{H3} domains, and wherein only one of the C_{H3} domains comprises amino acid substitutions at positions corresponding to positions 435 and 436 of human IgG1 or IgG4 according to EU Index, wherein the amino acid substitutions are H435R and Y436F. In some embodiments, the C_{H3} domains of the second and the third polypeptide chains are human IgG1 C_{H3} domains, and wherein the C_{H3} domains each comprise amino acid substitutions at positions corresponding to positions 234 and 235 of human IgG1 according to EU Index, wherein the amino acid substitutions are L234A and L235A.

In some embodiments according to any one of the embodiments disclosed herein, the first polypeptide chain of the binding protein comprises an amino acid sequence that is at least 95%, at least 96%, at least 97%, at least 98%, at least 99%, or 100% identical to the amino acid sequence of SEQ ID NO:98, the second polypeptide chain of the binding protein comprises an amino acid sequence that is at least 95%, at least 96%, at least 97%, at least 98%, at least 99%, or 100% identical to the amino acid sequence of SEQ ID NO:99, the third polypeptide chain of the binding protein comprises an amino acid sequence that is at least 95%, at least 96%, at least 97%, at least 98%, at least 99%, or 100% identical to the amino acid sequence of SEQ ID NO:100, and the fourth polypeptide chain of the binding protein comprises an amino acid sequence that is at least 95%, at least 96%, at least 97%, at least 98%, at least 99%, or 100% identical to the amino acid sequence of SEQ ID NO:101.

Further provided herein is a kit of polynucleotides, comprising a first polynucleotide that encodes a first polypeptide chain of the binding protein according to any one of the above embodiments, a second polynucleotide that encodes a second polypeptide chain of the binding protein according to any one of the above embodiments, a third polynucleotide that encodes a third polypeptide chain of the binding protein according to any one of the above embodiments, and a fourth polynucleotide that encodes a fourth polypeptide chain of the binding protein according to any one of the above embodiments.

Further provided herein are antibodies that specifically bind an IL-4 polypeptide, wherein the antibody comprises a heavy chain variable (VH) domain and a light chain variable (VH) domain; wherein the VH domain comprises a CDR-H1 comprising the amino acid sequence of SEQ ID NO:1, a CDR-H2 comprising the amino acid sequence of SEQ ID NO:2, and a CDR-H3 comprising the amino acid sequence selected from the group consisting of SEQ ID NOs:3-5; and wherein the VL domain comprises a CDR-L1 comprising the amino acid sequence of SEQ ID NO:6 or 7, a CDR-L2 comprising the amino acid sequence of SEQ ID NO:8 or 9, and a CDR-L3 comprising the amino acid sequence of SEQ ID NO:10 or 11. Further provided herein are antibodies that specifically bind an IL-4 polypeptide, wherein the antibody comprises a heavy chain variable (VH) domain and a light chain variable (VH) domain; wherein the VH domain comprises a CDR-H1 comprising the amino acid sequence of SEQ ID NO:22, a CDR-H2 comprising the amino acid sequence of SEQ ID NO:23, and a CDR-H3 comprising the amino acid sequence selected from the group consisting of SEQ ID NOs:24-27; and wherein the VL domain comprises a CDR-L1 comprising the amino acid sequence of SEQ ID NO:6 or 7, a CDR-L2 comprising the amino acid sequence of SEQ ID NO:8 or 9, and a CDR-L3 comprising the amino acid sequence of SEQ ID NO:10 or 11. In some embodiments, the VH domain comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3, and the VL domain comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10. In some embodiments, the VH domain comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3, and the VL domain comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11. In some embodiments, the VH domain comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:4, and the VL domain comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11. In some embodiments, the VH domain comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:5, and the VL domain comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11. In some embodiments, the VH domain comprises an amino acid sequence that is at least 95%, at least 96%, at least 97%, at least 98%, at least 99%, or 100% identical to the amino acid sequence of SEQ ID NO:34, and the VL domain comprises an amino acid sequence that is at least 95%, at least 96%, at least 97%, at least 98%, at least 99%, or 100% identical to the amino acid sequence of SEQ ID NO:35. In some embodiments, the VH domain comprises an amino acid sequence that is at least 95%, at least 96%, at least 97%, at least 98%, at least 99%, or 100% identical to the amino acid sequence of SEQ ID NO:36, and the VL domain comprises an amino acid sequence that is at least 95%, at least 96%, at least 97%, at least 98%, at least 99%, or 100% identical to the amino acid sequence of SEQ ID NO:37. In some embodiments, the VH domain comprises an amino acid sequence that is at least 95%, at least 96%, at least 97%, at least 98%, at least 99%, or 100% identical to the amino acid sequence of SEQ ID NO:38, and the VL domain comprises an amino acid sequence that is at least 95%, at least 96%, at least 97%, at least 98%, at least 99%, or 100% identical to the amino acid sequence of SEQ ID NO:39. In some embodiments, the VH domain comprises an amino acid sequence that is at least 95%, at least 96%, at least 97%, at least 98%, at least 99%, or 100% identical to the amino acid sequence of SEQ ID NO:40, and the VL domain comprises an amino acid sequence that is at least 95%, at least 96%, at least 97%, at least 98%, at least 99%, or 100% identical to the amino acid sequence of SEQ ID NO:41. In some embodiments, the VH domain comprises an amino acid sequence that is at least 95%, at least 96%, at least 97%, at least 98%, at least 99%, or 100% identical to the amino acid sequence of SEQ ID NO:42, and the VL domain comprises an amino acid sequence that is at least 95%, at least 96%, at least 97%, at least 98%, at least 99%, or 100% identical to the amino acid sequence of SEQ ID NO:43. In some embodiments, the VH domain comprises an amino acid sequence that is at least 95%, at least 96%, at least 97%, at least 98%, at least 99%, or 100% identical to the amino acid sequence of SEQ ID NO:44, and the VL domain comprises an amino acid sequence that is at least 95%, at least 96%, at least 97%, at least 98%, at least 99%, or 100% identical to the amino acid sequence of SEQ ID NO:43. In some embodiments, the VH domain comprises an amino acid sequence that is at least 95%, at least 96%, at least 97%, at least 98%, at least 99%, or 100% identical to the amino acid sequence of SEQ ID NO:45, and the VL domain comprises an amino acid sequence that is at least 95%, at least 96%, at least 97%, at least 98%, at least 99%, or 100% identical to the amino acid sequence of SEQ ID NO:43. In some embodiments, the VH domain comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24, and the VL domain comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10. In some embodiments, the VH domain comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24, and the VL domain comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11. In some embodiments, the VH domain comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, and the VL domain comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10. In some embodiments, the VH domain comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, and the VL domain comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11. In some embodiments, the VH domain comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:26, and the VL domain comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO: 11. In some embodiments, the VH domain comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:27, and the VL domain comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11. In some embodiments, the VH domain comprises an amino acid sequence that is at least 95%, at least 96%, at least 97%, at least 98%, at least 99%, or 100% identical to the amino acid sequence of SEQ ID NO:46, and the VL domain comprises an amino acid sequence that is at least 95%, at least 96%, at least 97%, at least 98%, at least 99%, or 100% identical to the amino acid sequence of SEQ ID NO:47. In some embodiments, the VH domain comprises an amino acid sequence that is at least 95%, at least 96%, at least 97%, at least 98%, at least 99%, or 100% identical to the amino acid sequence of SEQ ID NO:48, and the VL domain comprises an amino acid sequence that is at least 95%, at least 96%, at least 97%, at least 98%, at least 99%, or 100% identical to the amino acid sequence of SEQ ID NO:39. In some embodiments, the VH domain comprises an amino acid sequence that is at least 95%, at least 96%, at least 97%, at least 98%, at least 99%, or 100% identical to the amino acid sequence of SEQ ID NO:49, and the VL domain comprises an amino acid sequence that is at least 95%, at least 96%, at least 97%, at least 98%, at least 99%, or 100% identical to the amino acid sequence of SEQ ID NO:41. In some embodiments, the VH domain comprises an amino acid sequence that is at least 95%, at least 96%, at least 97%, at least 98%, at least 99%, or 100% identical to the amino acid sequence of SEQ ID NO:50, and the VL domain comprises an amino acid sequence that is at least 95%, at least 96%, at least 97%, at least 98%, at least 99%, or 100% identical to the amino acid sequence of SEQ ID NO:43. In some embodiments, the VH domain comprises an amino acid sequence that is at least 95%, at least 96%, at least 97%, at least 98%, at least 99%, or 100% identical to the amino acid sequence of SEQ ID NO:51, and the VL domain comprises an amino acid sequence that is at least 95%, at least 96%, at least 97%, at least 98%, at least 99%, or 100% identical to the amino acid sequence of SEQ ID NO:37. In some embodiments, the VH domain comprises an amino acid sequence that is at least 95%, at least 96%, at least 97%, at least 98%, at least 99%, or 100% identical to the amino acid sequence of SEQ ID NO:52, and the VL domain comprises an amino acid sequence that is at least 95%, at least 96%, at least 97%, at least 98%, at least 99%, or 100% identical to the amino acid sequence of SEQ ID NO:39. In some embodiments, the VH domain comprises an amino acid sequence that is at least 95%, at least 96%, at least 97%, at least 98%, at least 99%, or 100% identical to the amino acid sequence of SEQ ID NO:53, and the VL domain comprises an amino acid sequence that is at least 95%, at least 96%, at least 97%, at least 98%, at least 99%, or 100% identical to the amino acid sequence of SEQ ID NO:41. In some embodiments, the VH domain comprises an amino acid sequence that is at least 95%, at least 96%, at least 97%, at least 98%, at least 99%, or 100% identical to the amino acid sequence of SEQ ID NO:54, and the VL domain comprises an amino acid sequence that is at least 95%, at least 96%, at least 97%, at least 98%, at least 99%, or 100% identical to the amino acid sequence of SEQ ID NO:43. In some embodiments, the VH domain comprises an amino acid sequence that is at least 95%, at least 96%, at least 97%, at least 98%, at least 99%, or 100% identical to the amino acid sequence of SEQ ID NO:55, and the VL domain comprises an amino acid sequence that is at least 95%, at least 96%, at least 97%, at least 98%, at least 99%, or 100% identical to the amino acid sequence of SEQ ID NO:43. In some embodiments, the VH domain comprises an amino acid sequence that is at least 95%, at least 96%, at least 97%, at least 98%, at least 99%, or 100% identical to the amino acid sequence of SEQ ID NO:56, and the VL domain comprises an amino acid sequence that is at least 95%, at least 96%, at least 97%, at least 98%, at least 99%, or 100% identical to the amino acid sequence of SEQ ID NO:43. In some embodiments, the antibody is a monospecific antibody. In some embodiments, the antibody is a bispecific or multispecific antibody.

Further provided herein are antibodies that specifically bind an IL-13 polypeptide, wherein the antibody comprises a heavy chain variable (VH) domain and a light chain variable (VH) domain; wherein the VH domain comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, and the VL domain comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62. In some embodiments, the VH domain comprises an amino acid sequence that is at least 95%, at least 96%, at least 97%, at least 98%, at least 99%, or 100% identical to the amino acid sequence of SEQ ID NO:70, and the VL domain comprises an amino acid sequence that is at least 95%, at least 96%, at least 97%, at least 98%, at least 99%, or 100% identical to the amino acid sequence of SEQ ID NO:71. In some embodiments, the VH domain comprises an amino acid sequence that is at least 95%, at least 96%, at least 97%, at least 98%, at least 99%, or 100% identical to the amino acid sequence of SEQ ID NO:72, and the VL domain comprises an amino acid sequence that is at least 95%, at least 96%, at least 97%, at least 98%, at least 99%, or 100% identical to the amino acid sequence of SEQ ID NO:73. In some embodiments, the VH domain comprises an amino acid sequence that is at least 95%, at least 96%, at least 97%, at least 98%, at least 99%, or 100% identical to the amino acid sequence of SEQ ID NO:74, and the VL domain comprises an amino acid sequence that is at least 95%, at least 96%, at least 97%, at least 98%, at least 99%, or 100% identical to the amino acid sequence of SEQ ID NO:75. In some embodiments, the VH domain comprises an amino acid sequence that is at least 95%, at least 96%, at least 97%, at least 98%, at least 99%, or 100% identical to the amino acid sequence of SEQ ID NO:76, and the VL domain comprises an amino acid sequence that is at least 95%, at least 96%, at least 97%, at least 98%, at least 99%, or 100% identical to the amino acid sequence of SEQ ID NO:77. In some embodiments, the antibody is a monospecific antibody. In some embodiments, the antibody is a bispecific or multispecific antibody.

Further provided herein are antibodies that specifically bind an OX40L polypeptide, wherein the antibody comprises a heavy chain variable (VH) domain and a light chain variable (VH) domain; wherein the VH domain comprises a CDR-H1 comprising the amino acid sequence of SEQ ID NO:78, a CDR-H2 comprising the amino acid sequence of SEQ ID NO:79, and a CDR-H3 comprising the amino acid sequence of SEQ ID NO:80, and the VL domain comprises a CDR-L1 comprising the amino acid sequence of SEQ ID NO:81, a CDR-L2 comprising the amino acid sequence of SEQ ID NO:82, and a CDR-L3 comprising the amino acid sequence of SEQ ID NO:83. In some embodiments, the VH domain comprises an amino acid sequence that is at least 95%, at least 96%, at least 97%, at least 98%, at least 99%, or 100% identical to the amino acid sequence of SEQ ID NO:91, and the VL domain comprises an amino acid sequence that is at least 95%, at least 96%, at least 97%, at least 98%, at least 99%, or 100% identical to the amino acid sequence of SEQ ID NO:92. In some embodiments, the VH domain comprises an amino acid sequence that is at least 95%, at least 96%, at least 97%, at least 98%, at least 99%, or 100% identical to the amino acid sequence of SEQ ID NO:93, and the VL domain comprises an amino acid sequence that is at least 95%, at least 96%, at least 97%, at least 98%, at least 99%, or 100% identical to the amino acid sequence of SEQ ID NO:94. In some embodiments, the VH domain comprises an amino acid sequence that is at least 95%, at least 96%, at least 97%, at least 98%, at least 99%, or 100% identical to the amino acid sequence of SEQ ID NO:95, and the VL domain comprises an amino acid sequence that is at least 95%, at least 96%, at least 97%, at least 98%, at least 99%, or 100% identical to the amino acid sequence of SEQ ID NO:96. In some embodiments, the VH domain comprises an amino acid sequence that is at least 95%, at least 96%, at least 97%, at least 98%, at least 99%, or 100% identical to the amino acid sequence of SEQ ID NO:97, and the VL domain comprises an amino acid sequence that is at least 95%, at least 96%, at least 97%, at least 98%, at least 99%, or 100% identical to the amino acid sequence of SEQ ID NO:96. In some embodiments, the antibody is a monospecific antibody. In some embodiments, the antibody is a bispecific or multispecific antibody.

Further provided herein are trispecific, trivalent, multispecific and/or multivalent binding proteins comprising four polypeptide chains that form three antigen binding sites, wherein a first polypeptide chain of the binding protein comprises a structure represented by the formula:

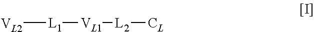

and a second polypeptide chain of the binding protein comprises a structure represented by the formula:

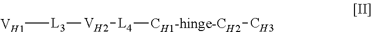

and a third polypeptide chain of the binding protein comprises a structure represented by the formula:

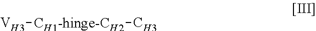

and a fourth polypeptide chain of the binding protein comprises a structure represented by the formula:

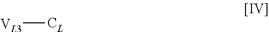

wherein:
   V_{L1} is a first immunoglobulin light chain variable domain;
   V_{L2} is a second immunoglobulin light chain variable domain;
   V_{L3} is a third immunoglobulin light chain variable domain;
   V_{H1} is a first immunoglobulin heavy chain variable domain;
   V_{H2} is a second immunoglobulin heavy chain variable domain;
   V_{H3} is a third immunoglobulin heavy chain variable domain;
   C_{L} is an immunoglobulin light chain constant domain;
   C_{H1} is an immunoglobulin C_{H1} heavy chain constant domain;
   C_{H2} is an immunoglobulin C_{H2} heavy chain constant domain;
   C_{H3} is an immunoglobulin C_{H3} heavy chain constant domain;
   hinge is an immunoglobulin hinge region connecting the C_{H1} and C_{H2} domains; and
   L₁, L₂, L₃ and L₄ are each independently amino acid linkers or zero amino acids in length;
wherein the polypeptide of formula I and the polypeptide of formula II form a cross-over light chain-heavy chain pair of the binding protein;
wherein V_{H1} and V_{L1} form a binding pair and a first antigen binding site, V_{H2} and V_{L2} form a binding pair and a second antigen binding site, and V_{H3} and V_{L3} form a binding pair and a third antigen binding site; and
wherein one of the three antigen binding sites specifically binds an interleukin-17 (IL-17) polypeptide and comprises: (a) a heavy chain variable (VH) domain that comprises a CDR-H1 comprising the amino acid sequence of SEQ ID NO: 117, a CDR-H2 comprising the amino acid sequence of SEQ ID NO:118, and a CDR-H3 comprising the amino acid sequence of SEQ ID NO:119, and a light chain variable (VL) domain that comprises a CDR-L1 comprising the amino acid sequence of SEQ ID NO:114, a CDR-L2 comprising the amino acid sequence of TFF, and a CDR-L3 comprising the amino acid sequence of SEQ ID NO:116; (b) a heavy chain variable (VH) domain that comprises a CDR-H1 comprising the amino acid sequence of SEQ ID NO:123, a CDR-H2 comprising the amino acid sequence of SEQ ID NO:124, and a CDR-H3 comprising the amino acid sequence of SEQ ID NO:125, and a light chain variable (VL) domain that comprises a CDR-L1 comprising the amino acid sequence of SEQ ID NO:120, a CDR-L2 comprising the amino acid sequence of GTY, and a CDR-L3 comprising the amino acid sequence of SEQ ID NO:122; (c) a heavy chain variable (VH) domain that comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131, and a light chain variable (VL) domain that comprises a CDR-L1 comprising the amino acid sequence of SEQ ID NO:126, a CDR-L2 comprising the amino acid sequence of GTY, and a CDR-L3 comprising the amino acid sequence of SEQ ID NO:128; (d) a VH domain that contains the same CDR-H1 sequence, the same CDR-H2 sequence, and/or the same CDR-H3 sequence, as the amino acid sequence of SEQ ID NO:132 and a VL domain that contains the same CDR-L1 sequence, the same CDR-L2 sequence, and/or the same CDR-L3 sequence, as the amino acid sequence of SEQ ID NO: 133; (e) a VH domain that contains the same CDR-H1 sequence, the same CDR-H2 sequence, and/or the same CDR-H3 sequence, as the amino acid sequence of SEQ ID NO:134 and a VL domain that contains the same CDR-L1 sequence, the same CDR-L2 sequence, and/or the same CDR-L3 sequence, as the amino acid sequence of SEQ ID NO:135; or (f) a VH domain that contains the same CDR-H1 sequence, the same CDR-H2 sequence, and/or the same CDR-H3 sequence, as the amino acid sequence of SEQ ID NO:136 and a VL domain that contains the same CDR-L1 sequence, the same CDR-L2 sequence, and/or the same CDR-L3 sequence, as the amino acid sequence of SEQ ID NO:137.

In some embodiments according to any one of the embodiments disclosed herein, the antigen binding site that specifically binds an IL-17 polypeptide is the third antigen binding site formed by V_{H3} and V_{L3}. In some embodiments, the antigen binding site that specifically binds an IL-17 polypeptide comprises a VH domain that comprises an amino acid sequence that is at least 95%, at least 96%, at least 97%, at least 98%, at least 99%, or 100% identical to the amino acid sequence of SEQ ID NO:132 and a VL domain that comprises an amino acid sequence that is at least 95%, at least 96%, at least 97%, at least 98%, at least 99%, or 100% identical to the amino acid sequence of SEQ ID NO:133. In some embodiments, the antigen binding site that specifically binds an IL-17 polypeptide comprises a VH domain that comprises an amino acid sequence that is at least 95%, at least 96%, at least 97%, at least 98%, at least 99%, or 100% identical to the amino acid sequence of SEQ ID NO:134 and a VL domain that comprises an amino acid sequence that is at least 95%, at least 96%, at least 97%, at least 98%, at least 99%, or 100% identical to the amino acid sequence of SEQ ID NO: 135. In some embodiments, the antigen binding site that specifically binds an IL-17 polypeptide comprises a VH domain that comprises an amino acid sequence that is at least 95%, at least 96%, at least 97%, at least 98%, at least 99%, or 100% identical to the amino acid sequence of SEQ ID NO:136 and a VL domain that comprises an amino acid sequence that is at least 95%, at least 96%, at least 97%, at least 98%, at least 99%, or 100% identical to the amino acid sequence of SEQ ID NO:137. In some embodiments, the first antigen binding site specifically binds an interleukin-4 (IL-4) polypeptide and the second antigen binding site specifically binds an interleukin-13 (IL-13) polypeptide. In some embodiments, the first antigen binding site specifically binds an IL-13 polypeptide and the second antigen binding site specifically binds an IL-4 polypeptide. In some embodiments, the binding site that specifically binds an IL-4 polypeptide comprises a VH domain that comprises a CDR-H1 comprising the amino acid sequence of SEQ ID NO:12, a CDR-H2 comprising the amino acid sequence of SEQ ID NO:13, and a CDR-H3 comprising the amino acid sequence of SEQ ID NO:14, and a VL domain that comprises a CDR-L1 comprising the amino acid sequence of SEQ ID NO:18, a CDR-L2 comprising the amino acid sequence of DAS, and a CDR-L3 comprising the amino acid sequence of SEQ ID NO:21. In some embodiments, the binding site that specifically binds an IL-4 polypeptide comprises a VH domain that comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:31, and a VL domain that comprises a CDR-L1 comprising the amino acid sequence of SEQ ID NO:18, a CDR-L2 comprising the amino acid sequence of DAS, and a CDR-L3 comprising the amino acid sequence of SEQ ID NO:21. In some embodiments, the binding site that specifically binds an IL-4 polypeptide comprises a VH domain that contains the same CDR-H1 sequence, the same CDR-H2 sequence, and/or the same CDR-H3 sequence, as the amino acid sequence of SEQ ID NO:40 and a VL domain that contains the same CDR-L1 sequence, the same CDR-L2 sequence, and/or the same CDR-L3 sequence, as the amino acid sequence of SEQ ID NO:41. In some embodiments, the binding site that specifically binds an IL-4 polypeptide comprises a VH domain that contains the same CDR-H1 sequence, the same CDR-H2 sequence, and/or the same CDR-H3 sequence, as the amino acid sequence of SEQ ID NO:53 and a VL domain that contains the same CDR-L1 sequence, the same CDR-L2 sequence, and/or the same CDR-L3 sequence, as the amino acid sequence of SEQ ID NO:41. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:40 and a VL domain that comprises an amino acid sequence that is at least 95%, at least 96%, at least 97%, at least 98%, at least 99%, or 100% identical to the amino acid sequence of SEQ ID NO:41. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:53 and a VL domain that comprises an amino acid sequence that is at least 95%, at least 96%, at least 97%, at least 98%, at least 99%, or 100% identical to the amino acid sequence of SEQ ID NO:41. In some embodiments, the binding site that specifically binds an IL-13 polypeptide comprises a VH domain that comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65, and a VL domain that comprises a CDR-L1 comprising the amino acid sequence of SEQ ID NO:67, a CDR-L2 comprising the amino acid sequence of KDS, and a CDR-L3 comprising the amino acid sequence of SEQ ID NO:69. In some embodiments, the binding site that specifically binds an IL-13 polypeptide comprises a VH domain that contains the same CDR-H1 sequence, the same CDR-H2 sequence, and/or the same CDR-H3 sequence, as the amino acid sequence of SEQ ID NO:72 and a VL domain that contains the same CDR-L1 sequence, the same CDR-L2 sequence, and/or the same CDR-L3 sequence, as the amino acid sequence of SEQ ID NO:73. In some embodiments, the binding site that specifically binds an IL-13 polypeptide comprises a VH domain that comprises an amino acid sequence that is at least 95%, at least 96%, at least 97%, at least 98%, at least 99%, or 100% identical to the amino acid sequence of SEQ ID NO:72 and a VL domain that comprises an amino acid sequence that is at least 95%, at least 96%, at least 97%, at least 98%, at least 99%, or 100% identical to the amino acid sequence of SEQ ID NO:73. In some embodiments, V_{H1} comprises a CDR-H1 comprising the amino acid sequence of SEQ ID NO:12, a CDR-H2 comprising the amino acid sequence of SEQ ID NO:13, and a CDR-H3 comprising the amino acid sequence of SEQ ID NO:14; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO:123, a CDR-H2 comprising the amino acid sequence of SEQ ID NO:124, and a CDR-H3 comprising the amino acid sequence of SEQ ID NO:125; and V_{L3} comprises a CDR-L1 comprising the amino acid sequence of SEQ ID NO:120, a CDR-L2 comprising the amino acid sequence of GTY, and a CDR-L3 comprising the amino acid sequence of SEQ ID NO:122. In some embodiments, V_{H1} contains the same CDR-H1 sequence, the same CDR-H2 sequence, and/or the same CDR-H3 sequence, as the amino acid sequence of SEQ ID NO:40 and V_{L1} contains the same CDR-L1 sequence, the same CDR-L2 sequence, and/or the same CDR-L3 sequence, as the amino acid sequence of SEQ ID NO:41; V_{H2} contains the same CDR-H1 sequence, the same CDR-H2 sequence, and/or the same CDR-H3 sequence, as the amino acid sequence of SEQ ID NO:72 and V_{L2} contains the same CDR-L1 sequence, the same CDR-L2 sequence, and/or the same CDR-L3 sequence, as the amino acid sequence of SEQ ID NO:73; and V_{H3} contains the same CDR-H1 sequence, the same CDR-H2 sequence, and/or the same CDR-H3 sequence, as the amino acid sequence of SEQ ID NO:134 and V_{L3} contains the same CDR-L1 sequence, the same CDR-L2 sequence, and/or the same CDR-L3 sequence, as the amino acid sequence of SEQ ID NO:135. In some embodiments, V_{H1} comprises a CDR-H1 comprising the amino acid sequence of SEQ ID NO:12, a CDR-H2 comprising the amino acid sequence of SEQ ID NO:13, and a CDR-H3 comprising the amino acid sequence of SEQ ID NO:14; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO:117, a CDR-H2 comprising the amino acid sequence of SEQ ID NO:118, and a CDR-H3 comprising the amino acid sequence of SEQ ID NO:119; and V_{L3} comprises a CDR-L1 comprising the amino acid sequence of SEQ ID NO: 114, a CDR-L2 comprising the amino acid sequence of TFF, and a CDR-L3 comprising the amino acid sequence of 116. In some embodiments, V_{H1} contains the same CDR-H1 sequence, the same CDR-H2 sequence, and/or the same CDR-H3 sequence, as the amino acid sequence of SEQ ID NO:40 and V_{L1} contains the same CDR-L1 sequence, the same CDR-L2 sequence, and/or the same CDR-L3 sequence, as the amino acid sequence of SEQ ID NO:41; V_{H2} contains the same CDR-H1 sequence, the same CDR-H2 sequence, and/or the same CDR-H3 sequence, as the amino acid sequence of SEQ ID NO:72 and V_{L2} contains the same CDR-L1 sequence, the same CDR-L2 sequence, and/or the same CDR-L3 sequence, as the amino acid sequence of SEQ ID NO:73; and V_{H3} contains the same CDR-H1 sequence, the same CDR-H2 sequence, and/or the same CDR-H3 sequence, as the amino acid sequence of SEQ ID NO:132 and V_{L3} contains the same CDR-L1 sequence, the same CDR-L2 sequence, and/or the same CDR-L3 sequence, as the amino acid sequence of SEQ ID NO:133. In some embodiments, V_{H1} comprises a CDR-H1 comprising the amino acid sequence of SEQ ID NO:12, a CDR-H2 comprising the amino acid sequence of SEQ ID NO:13, and a CDR-H3 comprising the amino acid sequence of SEQ ID NO:14; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO: 129, a CDR-H2 comprising the amino acid sequence of SEQ ID NO:130, and a CDR-H3 comprising the amino acid sequence of SEQ ID NO:131; and V_{L3} comprises a CDR-L1 comprising the amino acid sequence of SEQ ID NO: 126, a CDR-L2 comprising the amino acid sequence of GTY, and a CDR-L3 comprising the amino acid sequence of SEQ ID NO:128. In some embodiments, V_{H1} contains the same CDR-H1 sequence, the same CDR-H2 sequence, and/or the same CDR-H3 sequence, as the amino acid sequence of SEQ ID NO:40 and V_{L1} contains the same CDR-L1 sequence, the same CDR-L2 sequence, and/or the same CDR-L3 sequence, as the amino acid sequence of SEQ ID NO:41; V_{H2} contains the same CDR-H1 sequence, the same CDR-H2 sequence, and/or the same CDR-H3 sequence, as the amino acid sequence of SEQ ID NO:72 and V_{L2} contains the same CDR-L1 sequence, the same CDR-L2 sequence, and/or the same CDR-L3 sequence, as the amino acid sequence of SEQ ID NO:73; and V_{H3} contains the same CDR-H1 sequence, the same CDR-H2 sequence, and/or the same CDR-H3 sequence, as the amino acid sequence of SEQ ID NO:136 and V_{L3} contains the same CDR-L1 sequence, the same CDR-L2 sequence, and/or the same CDR-L3 sequence, as the amino acid sequence of SEQ ID NO:137. In some embodiments, V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:31; V_{L1} comprises a CDR-L1 comprising the amino acid sequence of SEQ ID NO: 18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO: 117, a CDR-H2 comprising the amino acid sequence of SEQ ID NO:118, and a CDR-H3 comprising the amino acid sequence of SEQ ID NO:119; and V_{L3} comprises a CDR-L1 comprising the amino acid sequence of SEQ ID NO: 114, a CDR-L2 comprising the amino acid sequence of TFF, and a CDR-L3 comprising the amino acid sequence of SEQ ID NO:116. In some embodiments, V_{H1} contains the same CDR-H1 sequence, the same CDR-H2 sequence, and/or the same CDR-H3 sequence, as the amino acid sequence of SEQ ID NO:53 and V_{L1} contains the same CDR-L1 sequence, the same CDR-L2 sequence, and/or the same CDR-L3 sequence, as the amino acid sequence of SEQ ID NO:41; V_{H2} contains the same CDR-H1 sequence, the same CDR-H2 sequence, and/or the same CDR-H3 sequence, as the amino acid sequence of SEQ ID NO:72 and V_{L2} contains the same CDR-L1 sequence, the same CDR-L2 sequence, and/or the same CDR-L3 sequence, as the amino acid sequence of SEQ ID NO:73; and V_{H3} contains the same CDR-H1 sequence, the same CDR-H2 sequence, and/or the same CDR-H3 sequence, as the amino acid sequence of SEQ ID NO:132 and V_{L3} contains the same CDR-L1 sequence, the same CDR-L2 sequence, and/or the same CDR-L3 sequence, as the amino acid sequence of SEQ ID NO:133. In some embodiments, V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:31; V_{L1} comprises a CDR-L1 comprising the amino acid sequence of SEQ ID NO: 18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO: 123, a CDR-H2 comprising the amino acid sequence of SEQ ID NO:124, and a CDR-H3 comprising the amino acid sequence of SEQ ID NO:125; and V_{L3} comprises a CDR-L1 comprising the amino acid sequence of SEQ ID NO: 120, a CDR-L2 comprising the amino acid sequence of GTY, and a CDR-L3 comprising the amino acid sequence of SEQ ID NO:122. In some embodiments, V_{H1} contains the same CDR-H1 sequence, the same CDR-H2 sequence, and/or the same CDR-H3 sequence, as the amino acid sequence of SEQ ID NO:53 and V_{L1} contains the same CDR-L1 sequence, the same CDR-L2 sequence, and/or the same CDR-L3 sequence, as the amino acid sequence of SEQ ID NO:41; V_{H2} contains the same CDR-H1 sequence, the same CDR-H2 sequence, and/or the same CDR-H3 sequence, as the amino acid sequence of SEQ ID NO:72 and V_{L2} contains the same CDR-L1 sequence, the same CDR-L2 sequence, and/or the same CDR-L3 sequence, as the amino acid sequence of SEQ ID NO:73; and V_{H3} contains the same CDR-H1 sequence, the same CDR-H2 sequence, and/or the same CDR-H3 sequence, as the amino acid sequence of SEQ ID NO:134 and V_{L3} contains the same CDR-L1 sequence, the same CDR-L2 sequence, and/or the same CDR-L3 sequence, as the amino acid sequence of SEQ ID NO:135. In some embodiments, V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:31; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO: 67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; and V_{L3} comprises a CDR-L1 comprising the amino acid sequence of SEQ ID NO: 126, a CDR-L2 comprising the amino acid sequence of GTY, and a CDR-L3 comprising the amino acid sequence of SEQ ID NO: 128. In some embodiments, V_{H1} contains the same CDR-H1 sequence, the same CDR-H2 sequence, and/or the same CDR-H3 sequence, as the amino acid sequence of SEQ ID NO:53 and V_{L1} contains the same CDR-L1 sequence, the same CDR-L2 sequence, and/or the same CDR-L3 sequence, as the amino acid sequence of SEQ ID NO:41; V_{H2} contains the same CDR-H1 sequence, the same CDR-H2 sequence, and/or the same CDR-H3 sequence, as the amino acid sequence of SEQ ID NO:72 and V_{L2} contains the same CDR-L1 sequence, the same CDR-L2 sequence, and/or the same CDR-L3 sequence, as the amino acid sequence of SEQ ID NO:73; and V_{H3} contains the same CDR-H1 sequence, the same CDR-H2 sequence, and/or the same CDR-H3 sequence, as the amino acid sequence of SEQ ID NO:136 and V_{L3} contains the same CDR-L1 sequence, the same CDR-L2 sequence, and/or the same CDR-L3 sequence, as the amino acid sequence of SEQ ID NO:137. In some embodiments, one, two, or all three of the antigen binding sites comprise reciprocal amino acid substitutions on the VH and VL domains that facilitate heavy-light chain pairing. In some embodiments, V_{H1} comprises an amino acid sequence that is at least 95%, at least 96%, at least 97%, at least 98%, at least 99%, or 100% identical to the amino acid sequence of SEQ ID NO:40; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:136; and V_{L3} comprises an amino acid sequence that is at least 95%, at least 96%, at least 97%, at least 98%, at least 99%, or 100% identical to the amino acid sequence of SEQ ID NO: 137. In some embodiments, V_{H1} comprises an amino acid sequence that is at least 95%, at least 96%, at least 97%, at least 98%, at least 99%, or 100% identical to the amino acid sequence of SEQ ID NO:40; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:134; and V_{L3} comprises an amino acid sequence that is at least 95%, at least 96%, at least 97%, at least 98%, at least 99%, or 100% identical to the amino acid sequence of SEQ ID NO:135. In some embodiments, V_{H1} comprises an amino acid sequence that is at least 95%, at least 96%, at least 97%, at least 98%, at least 99%, or 100% identical to the amino acid sequence of SEQ ID NO:40; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:132; and V_{L3} comprises an amino acid sequence that is at least 95%, at least 96%, at least 97%, at least 98%, at least 99%, or 100% identical to the amino acid sequence of SEQ ID NO:133. In some embodiments, V_{H1} comprises an amino acid sequence that is at least 95%, at least 96%, at least 97%, at least 98%, at least 99%, or 100% identical to the amino acid sequence of SEQ ID NO:53; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:132; and V_{L3} comprises an amino acid sequence that is at least 95%, at least 96%, at least 97%, at least 98%, at least 99%, or 100% identical to the amino acid sequence of SEQ ID NO:133. In some embodiments, V_{H1} comprises an amino acid sequence that is at least 95%, at least 96%, at least 97%, at least 98%, at least 99%, or 100% identical to the amino acid sequence of SEQ ID NO:53; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO: 134; and V_{L3} comprises an amino acid sequence that is at least 95%, at least 96%, at least 97%, at least 98%, at least 99%, or 100% identical to the amino acid sequence of SEQ ID NO: 135. In some embodiments, V_{H1} comprises an amino acid sequence that is at least 95%, at least 96%, at least 97%, at least 98%, at least 99%, or 100% identical to the amino acid sequence of SEQ ID NO:53; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:136; and V_{L3} comprises an amino acid sequence that is at least 95%, at least 96%, at least 97%, at least 98%, at least 99%, or 100% identical to the amino acid sequence of SEQ ID NO:137. In some embodiments, the binding protein is capable of inhibiting the function of one or more target proteins, and wherein the one or more target proteins comprise an IL-17 polypeptide. In some embodiments, the binding protein is capable of inhibiting the function of an IL-4 polypeptide, an IL-13 polypeptide, and an IL-17 polypeptide. In some embodiments, at least one of L₁, L₂, L₃ or L₄, is independently 0 amino acids in length or L₁, L₂, L₃ or L₄ are each independently at least one amino acid in length. In some embodiments, L₁, L₂, L₃ and L₄ each independently are zero amino acids in length or comprise a sequence selected from the group consisting of GGGGSGGGGS (SEQ ID NO:102), GGGGSGGGGSGGGGS (SEQ ID NO:103), S, RT, TKGPS (SEQ ID NO:104), GQPKAAP (SEQ ID NO: 105), and GGSGSSGSGG (SEQ ID NO:106). In some embodiments, L₁, L₂, L₃ and L₄ each independently comprise a sequence selected from the group consisting of GGGGSGGGGS (SEQ ID NO:102), GGGGSGGGGSGGGGS (SEQ ID NO:103), S, RT, TKGPS (SEQ ID NO:104), GQPKAAP (SEQ ID NO: 105), and GGSGSSGSGG (SEQ ID NO:106). In some embodiments, L₁ comprises the sequence GQPKAAP (SEQ ID NO: 105), L₂ comprises the sequence TKGPS (SEQ ID NO:104), L3 comprises the sequence S, and L₄ comprises the sequence RT. In some embodiments, L₁ comprises the sequence GGGGSGGGGS (SEQ ID NO:102), L₂ comprises the sequence GGGGSGGGGS (SEQ ID NO:102), L₃ is 0 amino acids in length, and L₄ is 0 amino acids in length. In some embodiments, L₁ comprises the sequence GGSGSSGSGG (SEQ ID NO:106), L₂ comprises the sequence GGSGSSGSGG (SEQ ID NO:106), L₃ is 0 amino acids in length, and L₄ is 0 amino acids in length. In some embodiments, L₁ comprises the sequence GGGGSGGGGSGGGGS (SEQ ID NO:103), L₂ is 0 amino acids in length, L3 comprises the sequence GGGGSGGGGSGGGGS (SEQ ID NO: 103), and L₄ is 0 amino acids in length. In some embodiments, the C_{H3} domain of the second polypeptide chain comprises amino acid substitutions at positions corresponding to positions 354 and 366 of human IgG1 or IgG4 according to EU Index, optionally wherein the amino acid substitutions are S354C and T366W; and wherein the C_{H3} domain of the third polypeptide chain comprises amino acid substitutions at positions corresponding to positions 349, 366, 368, and 407 of human IgG1 or IgG4 according to EU Index, optionally wherein the amino acid substitutions are Y349C, T366S, L368A, and Y407V. In some embodiments, the C_{H3} domain of the second polypeptide chain comprises amino acid substitutions at positions corresponding to positions 349, 366, 368, and 407 of human IgG1 or IgG4 according to EU Index, optionally wherein the amino acid substitutions are Y349C, T366S, L368A, and Y407V; and wherein the C_{H3} domain of the third polypeptide chain comprises amino acid substitutions at positions corresponding to positions 354 and 366 of human IgG1 or IgG4 according to EU Index, optionally wherein the amino acid substitutions are S354C and T366W. In some embodiments, the C_{H3} domains of the second and the third polypeptide chains are human IgG1 or IgG4 C_{H3} domains, and wherein only one of the C_{H3} domains comprises amino acid substitutions at positions corresponding to positions 435 and 436 of human IgG1 or IgG4 according to EU Index, optionally wherein the amino acid substitutions are H435R and Y436F. In some embodiments, the C_{H3} domains of the second and the third polypeptide chains are human IgG1 C_{H3} domains, and wherein the C_{H3} domains each comprise amino acid substitutions at positions corresponding to positions 234 and 235 of human IgG1 according to EU Index, optionally wherein the amino acid substitutions are L234A and L235A. In some embodiments, the C_{H3} domains of the second and the third polypeptide chains are human IgG1 C_{H3} domains, and wherein the C_{H3} domains each comprise amino acid substitutions at positions corresponding to positions 256 and 307 of human IgG1 according to EU Index, optionally wherein the amino acid substitutions are T256D and T307Q. In some embodiments, the first polypeptide chain of the binding protein comprises an amino acid sequence that is at least 95%, at least 96%, at least 97%, at least 98%, at least 99%, or 100% identical to the amino acid sequence of SEQ ID NO:142, the second polypeptide chain of the binding protein comprises an amino acid sequence that is at least 95%, at least 96%, at least 97%, at least 98%, at least 99%, or 100% identical to the amino acid sequence of SEQ ID NO:143, the third polypeptide chain of the binding protein comprises an amino acid sequence that is at least 95%, at least 96%, at least 97%, at least 98%, at least 99%, or 100% identical to the amino acid sequence of SEQ ID NO:144, and the fourth polypeptide chain of the binding protein comprises an amino acid sequence that is at least 95%, at least 96%, at least 97%, at least 98%, at least 99%, or 100% identical to the amino acid sequence of SEQ ID NO:145. In some embodiments, the first polypeptide chain of the binding protein comprises an amino acid sequence that is at least 95%, at least 96%, at least 97%, at least 98%, at least 99%, or 100% identical to the amino acid sequence of SEQ ID NO:138, the second polypeptide chain of the binding protein comprises an amino acid sequence that is at least 95%, at least 96%, at least 97%, at least 98%, at least 99%, or 100% identical to the amino acid sequence of SEQ ID NO:139, the third polypeptide chain of the binding protein comprises an amino acid sequence that is at least 95%, at least 96%, at least 97%, at least 98%, at least 99%, or 100% identical to the amino acid sequence of SEQ ID NO:140, and the fourth polypeptide chain of the binding protein comprises an amino acid sequence that is at least 95%, at least 96%, at least 97%, at least 98%, at least 99%, or 100% identical to the amino acid sequence of SEQ ID NO:141. In some embodiments, the first polypeptide chain of the binding protein comprises an amino acid sequence that is at least 95%, at least 96%, at least 97%, at least 98%, at least 99%, or 100% identical to the amino acid sequence of SEQ ID NO: 146, the second polypeptide chain of the binding protein comprises an amino acid sequence that is at least 95%, at least 96%, at least 97%, at least 98%, at least 99%, or 100% identical to the amino acid sequence of SEQ ID NO:147, the third polypeptide chain of the binding protein comprises an amino acid sequence that is at least 95%, at least 96%, at least 97%, at least 98%, at least 99%, or 100% identical to the amino acid sequence of SEQ ID NO: 148, and the fourth polypeptide chain of the binding protein comprises an amino acid sequence that is at least 95%, at least 96%, at least 97%, at least 98%, at least 99%, or 100% identical to the amino acid sequence of SEQ ID NO:149. In some embodiments, the first polypeptide chain of the binding protein comprises an amino acid sequence that is at least 95%, at least 96%, at least 97%, at least 98%, at least 99%, or 100% identical to the amino acid sequence of SEQ ID NO: 150, the second polypeptide chain of the binding protein comprises an amino acid sequence that is at least 95%, at least 96%, at least 97%, at least 98%, at least 99%, or 100% identical to the amino acid sequence of SEQ ID NO:151, the third polypeptide chain of the binding protein comprises an amino acid sequence that is at least 95%, at least 96%, at least 97%, at least 98%, at least 99%, or 100% identical to the amino acid sequence of SEQ ID NO:152, and the fourth polypeptide chain of the binding protein comprises an amino acid sequence that is at least 95%, at least 96%, at least 97%, at least 98%, at least 99%, or 100% identical to the amino acid sequence of SEQ ID NO:153. In some embodiments, the first polypeptide chain of the binding protein comprises the amino acid sequence of SEQ ID NO:142, the second polypeptide chain of the binding protein comprises the amino acid sequence of SEQ ID NO:143, the third polypeptide chain of the binding protein comprises the amino acid sequence of SEQ ID NO:144, and the fourth polypeptide chain of the binding protein comprises the amino acid sequence of SEQ ID NO:145. In some embodiments, the first polypeptide chain of the binding protein comprises the amino acid sequence of SEQ ID NO:138, the second polypeptide chain of the binding protein comprises the amino acid sequence of SEQ ID NO:139, the third polypeptide chain of the binding protein comprises the amino acid sequence of SEQ ID NO:140, and the fourth polypeptide chain of the binding protein comprises the amino acid sequence of SEQ ID NO:141. In some embodiments, the first polypeptide chain of the binding protein comprises the amino acid sequence of SEQ ID NO:146, the second polypeptide chain of the binding protein comprises the amino acid sequence of SEQ ID NO:147, the third polypeptide chain of the binding protein comprises the amino acid sequence of SEQ ID NO:148, and the fourth polypeptide chain of the binding protein comprises the amino acid sequence of SEQ ID NO:149. In some embodiments, the first polypeptide chain of the binding protein comprises the amino acid sequence of SEQ ID NO:150, the second polypeptide chain of the binding protein comprises the amino acid sequence of SEQ ID NO:151, the third polypeptide chain of the binding protein comprises the amino acid sequence of SEQ ID NO:152, and the fourth polypeptide chain of the binding protein comprises the amino acid sequence of SEQ ID NO:153.

Further provided herein are antibodies that specifically bind an IL-17 polypeptide. In some embodiments, the antibodies comprise a heavy chain variable (VH) domain that comprises a CDR-H1 comprising the amino acid sequence of SEQ ID NO:117, a CDR-H2 comprising the amino acid sequence of SEQ ID NO:118, and a CDR-H3 comprising the amino acid sequence of SEQ ID NO:119, and a light chain variable (VL) domain that comprises a CDR-L1 comprising the amino acid sequence of SEQ ID NO:114, a CDR-L2 comprising the amino acid sequence of TFF, and a CDR-L3 comprising the amino acid sequence of SEQ ID NO:116. In some embodiments, the antibodies comprise a heavy chain variable (VH) domain that comprises a CDR-H1 comprising the amino acid sequence of SEQ ID NO:123, a CDR-H2 comprising the amino acid sequence of SEQ ID NO:124, and a CDR-H3 comprising the amino acid sequence of SEQ ID NO:125, and a light chain variable (VL) domain that comprises a CDR-L1 comprising the amino acid sequence of SEQ ID NO:120, a CDR-L2 comprising the amino acid sequence of GTY, and a CDR-L3 comprising the amino acid sequence of SEQ ID NO:122. In some embodiments, the antibodies comprise a heavy chain variable (VH) domain that comprises a CDR-H1 comprising the amino acid sequence of SEQ ID NO:129, a CDR-H2 comprising the amino acid sequence of SEQ ID NO: 130, and a CDR-H3 comprising the amino acid sequence of SEQ ID NO:131, and a light chain variable (VL) domain that comprises a CDR-L1 comprising the amino acid sequence of SEQ ID NO:126, a CDR-L2 comprising the amino acid sequence of GTY, and a CDR-L3 comprising the amino acid sequence of SEQ ID NO: 128. In some embodiments, the antibodies comprise a VH domain that contains the same CDR-H1 sequence, the same CDR-H2 sequence, and/or the same CDR-H3 sequence, as the amino acid sequence of SEQ ID NO:132 and a VL domain that contains the same CDR-L1 sequence, the same CDR-L2 sequence, and/or the same CDR-L3 sequence, as the amino acid sequence of SEQ ID NO:133. In some embodiments, the antibodies comprise a VH domain that contains the same CDR-H1 sequence, the same CDR-H2 sequence, and/or the same CDR-H3 sequence, as the amino acid sequence of SEQ ID NO:134 and a VL domain that contains the same CDR-L1 sequence, the same CDR-L2 sequence, and/or the same CDR-L3 sequence, as the amino acid sequence of SEQ ID NO:135. In some embodiments, the antibodies comprise a VH domain that contains the same CDR-H1 sequence, the same CDR-H2 sequence, and/or the same CDR-H3 sequence, as the amino acid sequence of SEQ ID NO:136 and a VL domain that contains the same CDR-L1 sequence, the same CDR-L2 sequence, and/or the same CDR-L3 sequence, as the amino acid sequence of SEQ ID NO:137. In some embodiments, the VH domain comprises an amino acid sequence that is at least 95%, at least 96%, at least 97%, at least 98%, at least 99%, or 100% identical to the amino acid sequence of SEQ ID NO:132 and the VL domain comprises an amino acid sequence that is at least 95%, at least 96%, at least 97%, at least 98%, at least 99%, or 100% identical to the amino acid sequence of 133. In some embodiments, the VH domain comprises an amino acid sequence that is at least 95%, at least 96%, at least 97%, at least 98%, at least 99%, or 100% identical to the amino acid sequence of SEQ ID NO: 134 and the VL domain comprises an amino acid sequence that is at least 95%, at least 96%, at least 97%, at least 98%, at least 99%, or 100% identical to the amino acid sequence of 135. In some embodiments, the VH domain comprises an amino acid sequence that is at least 95%, at least 96%, at least 97%, at least 98%, at least 99%, or 100% identical to the amino acid sequence of SEQ ID NO:136 and the VL domain comprises an amino acid sequence that is at least 95%, at least 96%, at least 97%, at least 98%, at least 99%, or 100% identical to the amino acid sequence of 137. In some embodiments, the antibodies are monospecific and/or monovalent. In some embodiments, the antibodies are bispecific and/or bivalent. In some embodiments, the antibodies are multispecific and/or multivalent.

In some embodiments, provided herein are trispecific binding proteins that form three antigen binding sites, wherein one of the antigen binding sites specifically binds an interleukin-4 (IL-4) polypeptide, one of the antigen binding sites specifically binds an interleukin-13 (IL-13) polypeptide, and one of the antigen binding sites specifically binds a polypeptide expressed on the surface of a cytotoxic lymphocyte (e.g. NK cells, CD4+ T cells, CD8+αβ T cells and γδ T cells). In some embodiments, the polypeptide is expressed on the surface of a natural killer (NK) cell. In some embodiments, one of the antigen binding sites specifically binds an IL-4 polypeptide, one of the antigen binding sites specifically binds an IL-13 polypeptide, and one of the antigen binding sites specifically binds a natural killer group 2D (NKG2D) polypeptide. In some embodiments, provided herein are trispecific binding proteins comprising four polypeptide chains that form three antigen binding sites, wherein a first polypeptide chain of the binding protein comprises a structure represented by the formula:

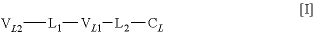

and a second polypeptide chain of the binding protein comprises a structure represented by the formula:

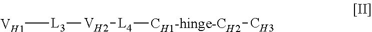

and a third polypeptide chain of the binding protein comprises a structure represented by the formula:

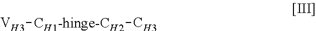

and a fourth polypeptide chain of the binding protein comprises a structure represented by the formula:
wherein:

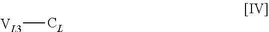

V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are amino acid linkers;
wherein the polypeptide of formula I and the polypeptide of formula II form a cross-over light chain-heavy chain pair;
wherein V_{H1} and V_{L1} form a binding pair and a first antigen binding site, V_{H2} and V_{L2} form a binding pair and a second antigen binding site, and V_{H3} and V_{L3} form a binding pair and a third antigen binding site; and wherein one of the three antigen binding sites specifically binds an interleukin-4 (IL-4) polypeptide, one of the three antigen binding sites specifically binds an interleukin-13 (IL-13) polypeptide, and one of the three antigen binding sites specifically binds a polypeptide. In some embodiments, the polypeptide is expressed on the surface of a natural killer (NK) cell. In some embodiments, the polypeptide is expressed on the surface of a CD4+ T cell. In some embodiments, the polypeptide is expressed on the surface of a CD8+αβ T cell. In some embodiments, the polypeptide is expressed on the surface of a γδ T cell. In some embodiments, the antigen is expressed on the surface of a natural killer (NK) cell. In some embodiments, one of the antigen binding sites specifically binds an IL-4 polypeptide, one of the antigen binding sites specifically binds an IL-13 polypeptide, and one of the antigen binding sites specifically binds a natural killer group 2D (NKG2D) polypeptide. In some embodiments, the first antigen binding site specifically binds the IL-4 polypeptide and the second antigen binding site specifically binds the IL-13 polypeptide. In some embodiments, the first antigen binding site specifically binds the IL-13 polypeptide and the second antigen binding site specifically binds the IL-4 polypeptide. In some embodiments, the antigen binding site that specifically binds the NKG2D polypeptide comprises a VH domain that comprises a CDR-H1 comprising the amino acid sequence of SEQ ID NO:157, a CDR-H2 comprising the amino acid sequence of SEQ ID NO:158, and a CDR-H3 comprising the amino acid sequence of SEQ ID NO: 159, and a VL domain that comprises a CDR-L1 comprising the amino acid sequence of SEQ ID NO:160, a CDR-L2 comprising the amino acid sequence of RDS, and a CDR-L3 comprising the amino acid sequence of SEQ ID NO:162. In some embodiments, the antigen binding site that specifically binds the NKG2D polypeptide comprises a VH domain that comprises a CDR-H1 comprising the amino acid sequence of SEQ ID NO:163, a CDR-H2 comprising the amino acid sequence of SEQ ID NO:164, and a CDR-H3 comprising the amino acid sequence of SEQ ID NO: 165, and a VL domain that comprises a CDR-L1 comprising the amino acid sequence of SEQ ID NO:166, a CDR-L2 comprising the amino acid sequence of YDD, and a CDR-L3 comprising the amino acid sequence of SEQ ID NO:168. In some embodiments, the antigen binding site that specifically binds the NKG2D polypeptide comprises a VH domain that comprises a CDR-H1 comprising the amino acid sequence of SEQ ID NO:169, a CDR-H2 comprising the amino acid sequence of SEQ ID NO:170, and a CDR-H3 comprising the amino acid sequence of SEQ ID NO:171, and a VL domain that comprises a CDR-L1 comprising the amino acid sequence of SEQ ID NO:172, a CDR-L2 comprising the amino acid sequence of YDD, and a CDR-L3 comprising the amino acid sequence of SEQ ID NO:174. In some embodiments, the antigen binding site that specifically binds the NKG2D polypeptide comprises a VH domain that comprises a CDR-H1 comprising the amino acid sequence of SEQ ID NO:175, a CDR-H2 comprising the amino acid sequence of SEQ ID NO:176, and a CDR-H3 comprising the amino acid sequence of SEQ ID NO: 177, and a VL domain that comprises a CDR-L1 comprising the amino acid sequence of SEQ ID NO:178, a CDR-L2 comprising the amino acid sequence of YDD, and a CDR-L3 comprising the amino acid sequence of SEQ ID NO:180. In some embodiments, the antigen binding site that specifically binds the NKG2D polypeptide comprises a VH domain that comprises a CDR-H1 comprising the amino acid sequence of SEQ ID NO:181, a CDR-H2 comprising the amino acid sequence of SEQ ID NO:182, and a CDR-H3 comprising the amino acid sequence of SEQ ID NO: 183, and a VL domain that comprises a CDR-L1 comprising the amino acid sequence of SEQ ID NO:184, a CDR-L2 comprising the amino acid sequence of QDT, and a CDR-L3 comprising the amino acid sequence of SEQ ID NO:186. In some embodiments, the antigen binding site that specifically binds the NKG2D polypeptide comprises a VH domain that comprises a CDR-H1 comprising the amino acid sequence of SEQ ID NO:187, a CDR-H2 comprising the amino acid sequence of SEQ ID NO:188, and a CDR-H3 comprising the amino acid sequence of SEQ ID NO: 189, and a VL domain that comprises a CDR-L1 comprising the amino acid sequence of SEQ ID NO:190, a CDR-L2 comprising the amino acid sequence of PAS, and a CDR-L3 comprising the amino acid sequence of SEQ ID NO:192. In some embodiments, the antigen binding site that specifically binds the NKG2D polypeptide comprises a VH domain that comprises a CDR-H1 comprising the amino acid sequence of SEQ ID NO:193, a CDR-H2 comprising the amino acid sequence of SEQ ID NO:194, and a CDR-H3 comprising the amino acid sequence of SEQ ID NO: 195, and a VL domain that comprises a CDR-L1 comprising the amino acid sequence of SEQ ID NO:196, a CDR-L2 comprising the amino acid sequence of ATS, and a CDR-L3 comprising the amino acid sequence of SEQ ID NO:198. In some embodiments, the antigen binding site that specifically binds the NKG2D polypeptide comprises a VH domain that comprises a CDR-H1 comprising the amino acid sequence of SEQ ID NO:199, a CDR-H2 comprising the amino acid sequence of SEQ ID NO:200, and a CDR-H3 comprising the amino acid sequence of SEQ ID NO:201, and a VL domain that comprises a CDR-L1 comprising the amino acid sequence of SEQ ID NO:202, a CDR-L2 comprising the amino acid sequence of PAS, and a CDR-L3 comprising the amino acid sequence of SEQ ID NO:204. In some embodiments, the antigen binding site that specifically binds the NKG2D polypeptide comprises a VH domain that comprises a CDR-H1 comprising the amino acid sequence of SEQ ID NO:205, a CDR-H2 comprising the amino acid sequence of SEQ ID NO:206, and a CDR-H3 comprising the amino acid sequence of SEQ ID NO:207, and a VL domain that comprises a CDR-L1 comprising the amino acid sequence of SEQ ID NO:208, a CDR-L2 comprising the amino acid sequence of AAS, and a CDR-L3 comprising the amino acid sequence of SEQ ID NO:210. In some embodiments, the antigen binding site that specifically binds the NKG2D polypeptide comprises a VH domain that comprises a CDR-H1 comprising the amino acid sequence of SEQ ID NO:211, a CDR-H2 comprising the amino acid sequence of SEQ ID NO:212, and a CDR-H3 comprising the amino acid sequence of SEQ ID NO:213, and a VL domain that comprises a CDR-L1 comprising the amino acid sequence of SEQ ID NO:214, a CDR-L2 comprising the amino acid sequence of YAS, and a CDR-L3 comprising the amino acid sequence of SEQ ID NO:216. In some embodiments, the antigen binding site that specifically binds the NKG2D polypeptide comprises a VH domain that comprises an amino acid sequence that is at least 95%, at least 96%, at least 97%, at least 98%, at least 99%, or 100% identical to the amino acid sequence of SEQ ID NO:217 and a VL domain that comprises an amino acid sequence that is at least 95%, at least 96%, at least 97%, at least 98%, at least 99%, or 100% identical to the amino acid sequence of SEQ ID NO:218. In some embodiments, the antigen binding site that specifically binds the NKG2D polypeptide comprises a VH domain that comprises an amino acid sequence that is at least 95%, at least 96%, at least 97%, at least 98%, at least 99%, or 100% identical to the amino acid sequence of SEQ ID NO:219 and a VL domain that comprises an amino acid sequence that is at least 95%, at least 96%, at least 97%, at least 98%, at least 99%, or 100% identical to the amino acid sequence of SEQ ID NO:220. In some embodiments, the antigen binding site that specifically binds the NKG2D polypeptide comprises a VH domain that comprises an amino acid sequence that is at least 95%, at least 96%, at least 97%, at least 98%, at least 99%, or 100% identical to the amino acid sequence of SEQ ID NO:221 and a VL domain that comprises an amino acid sequence that is at least 95%, at least 96%, at least 97%, at least 98%, at least 99%, or 100% identical to the amino acid sequence of SEQ ID NO:222. In some embodiments, the antigen binding site that specifically binds the NKG2D polypeptide comprises a VH domain that comprises an amino acid sequence that is at least 95%, at least 96%, at least 97%, at least 98%, at least 99%, or 100% identical to the amino acid sequence of SEQ ID NO:223 and a VL domain that comprises an amino acid sequence that is at least 95%, at least 96%, at least 97%, at least 98%, at least 99%, or 100% identical to the amino acid sequence of SEQ ID NO:224. In some embodiments, the antigen binding site that specifically binds the NKG2D polypeptide comprises a VH domain that comprises an amino acid sequence that is at least 95%, at least 96%, at least 97%, at least 98%, at least 99%, or 100% identical to the amino acid sequence of SEQ ID NO:225 and a VL domain that comprises an amino acid sequence that is at least 95%, at least 96%, at least 97%, at least 98%, at least 99%, or 100% identical to the amino acid sequence of SEQ ID NO:226. In some embodiments, the antigen binding site that specifically binds the NKG2D polypeptide comprises a VH domain that comprises an amino acid sequence that is at least 95%, at least 96%, at least 97%, at least 98%, at least 99%, or 100% identical to the amino acid sequence of SEQ ID NO:227 and a VL domain that comprises an amino acid sequence that is at least 95%, at least 96%, at least 97%, at least 98%, at least 99%, or 100% identical to the amino acid sequence of SEQ ID NO:228. In some embodiments, the antigen binding site that specifically binds the NKG2D polypeptide comprises a VH domain that comprises an amino acid sequence that is at least 95%, at least 96%, at least 97%, at least 98%, at least 99%, or 100% identical to the amino acid sequence of SEQ ID NO:229 and a VL domain that comprises an amino acid sequence that is at least 95%, at least 96%, at least 97%, at least 98%, at least 99%, or 100% identical to the amino acid sequence of SEQ ID NO:230. In some embodiments, the antigen binding site that specifically binds the NKG2D polypeptide comprises a VH domain that comprises an amino acid sequence that is at least 95%, at least 96%, at least 97%, at least 98%, at least 99%, or 100% identical to the amino acid sequence of SEQ ID NO:231 and a VL domain that comprises an amino acid sequence that is at least 95%, at least 96%, at least 97%, at least 98%, at least 99%, or 100% identical to the amino acid sequence of SEQ ID NO:232. In some embodiments, the antigen binding site that specifically binds the NKG2D polypeptide comprises a VH domain that comprises an amino acid sequence that is at least 95%, at least 96%, at least 97%, at least 98%, at least 99%, or 100% identical to the amino acid sequence of SEQ ID NO:233 and a VL domain that comprises an amino acid sequence that is at least 95%, at least 96%, at least 97%, at least 98%, at least 99%, or 100% identical to the amino acid sequence of SEQ ID NO:234. In some embodiments, the antigen binding site that specifically binds the NKG2D polypeptide comprises a VH domain that comprises an amino acid sequence that is at least 95%, at least 96%, at least 97%, at least 98%, at least 99%, or 100% identical to the amino acid sequence of SEQ ID NO:235 and a VL domain that comprises an amino acid sequence that is at least 95%, at least 96%, at least 97%, at least 98%, at least 99%, or 100% identical to the amino acid sequence of SEQ ID NO:236. In some embodiments, the antigen binding site that specifically binds the NKG2D polypeptide comprises a VH domain that comprises an amino acid sequence that is at least 95%, at least 96%, at least 97%, at least 98%, at least 99%, or 100% identical to the amino acid sequence of SEQ ID NO:237 and a VL domain that comprises an amino acid sequence that is at least 95%, at least 96%, at least 97%, at least 98%, at least 99%, or 100% identical to the amino acid sequence of SEQ ID NO:238. In some embodiments, the antigen binding site that specifically binds the NKG2D polypeptide comprises a VH domain that comprises an amino acid sequence that is at least 95%, at least 96%, at least 97%, at least 98%, at least 99%, or 100% identical to the amino acid sequence of SEQ ID NO:239 and a VL domain that comprises an amino acid sequence that is at least 95%, at least 96%, at least 97%, at least 98%, at least 99%, or 100% identical to the amino acid sequence of SEQ ID NO:238. In some embodiments, the antigen binding site that specifically binds the NKG2D polypeptide comprises a VH domain that comprises an amino acid sequence that is at least 95%, at least 96%, at least 97%, at least 98%, at least 99%, or 100% identical to the amino acid sequence of SEQ ID NO:240 and a VL domain that comprises an amino acid sequence that is at least 95%, at least 96%, at least 97%, at least 98%, at least 99%, or 100% identical to the amino acid sequence of SEQ ID NO:241. In some embodiments, V_{H3} comprises an amino acid sequence that is at least 95%, at least 96%, at least 97%, at least 98%, at least 99%, or 100% identical to the amino acid sequence of SEQ ID NO:217 and V_{L3} comprises an amino acid sequence that is at least 95%, at least 96%, at least 97%, at least 98%, at least 99%, or 100% identical to the amino acid sequence of SEQ ID NO:218. In some embodiments, V_{H3} comprises an amino acid sequence that is at least 95%, at least 96%, at least 97%, at least 98%, at least 99%, or 100% identical to the amino acid sequence of SEQ ID NO:219 and V_{L3} comprises an amino acid sequence that is at least 95%, at least 96%, at least 97%, at least 98%, at least 99%, or 100% identical to the amino acid sequence of SEQ ID NO:220. In some embodiments, V_{H3} comprises an amino acid sequence that is at least 95%, at least 96%, at least 97%, at least 98%, at least 99%, or 100% identical to the amino acid sequence of SEQ ID NO:221 and V_{L3} comprises an amino acid sequence that is at least 95%, at least 96%, at least 97%, at least 98%, at least 99%, or 100% identical to the amino acid sequence of SEQ ID NO:222. In some embodiments, V_{H3} comprises an amino acid sequence that is at least 95%, at least 96%, at least 97%, at least 98%, at least 99%, or 100% identical to the amino acid sequence of SEQ ID NO:223 and V_{L3} comprises an amino acid sequence that is at least 95%, at least 96%, at least 97%, at least 98%, at least 99%, or 100% identical to the amino acid sequence of SEQ ID NO:224. In some embodiments, V_{H3} comprises an amino acid sequence that is at least 95%, at least 96%, at least 97%, at least 98%, at least 99%, or 100% identical to the amino acid sequence of SEQ ID NO:225 and V_{L3} comprises an amino acid sequence that is at least 95%, at least 96%, at least 97%, at least 98%, at least 99%, or 100% identical to the amino acid sequence of SEQ ID NO:226. In some embodiments, V_{H3} comprises an amino acid sequence that is at least 95%, at least 96%, at least 97%, at least 98%, at least 99%, or 100% identical to the amino acid sequence of SEQ ID NO:227 and V_{L3} comprises an amino acid sequence that is at least 95%, at least 96%, at least 97%, at least 98%, at least 99%, or 100% identical to the amino acid sequence of SEQ ID NO:228. In some embodiments, V_{H3} comprises an amino acid sequence that is at least 95%, at least 96%, at least 97%, at least 98%, at least 99%, or 100% identical to the amino acid sequence of SEQ ID NO:229 and V_{L3} comprises an amino acid sequence that is at least 95%, at least 96%, at least 97%, at least 98%, at least 99%, or 100% identical to the amino acid sequence of SEQ ID NO:230. In some embodiments, V_{H3} comprises an amino acid sequence that is at least 95%, at least 96%, at least 97%, at least 98%, at least 99%, or 100% identical to the amino acid sequence of SEQ ID NO:231 and V_{L3} comprises an amino acid sequence that is at least 95%, at least 96%, at least 97%, at least 98%, at least 99%, or 100% identical to the amino acid sequence of SEQ ID NO:232. In some embodiments, V_{H3} comprises an amino acid sequence that is at least 95%, at least 96%, at least 97%, at least 98%, at least 99%, or 100% identical to the amino acid sequence of SEQ ID NO:233 and V_{L3} comprises an amino acid sequence that is at least 95%, at least 96%, at least 97%, at least 98%, at least 99%, or 100% identical to the amino acid sequence of SEQ ID NO:234. In some embodiments, V_{H3} comprises an amino acid sequence that is at least 95%, at least 96%, at least 97%, at least 98%, at least 99%, or 100% identical to the amino acid sequence of SEQ ID NO:235 and V_{L3} comprises an amino acid sequence that is at least 95%, at least 96%, at least 97%, at least 98%, at least 99%, or 100% identical to the amino acid sequence of SEQ ID NO:236. In some embodiments, V_{H3} comprises an amino acid sequence that is at least 95%, at least 96%, at least 97%, at least 98%, at least 99%, or 100% identical to the amino acid sequence of SEQ ID NO:237 and V_{L3} comprises an amino acid sequence that is at least 95%, at least 96%, at least 97%, at least 98%, at least 99%, or 100% identical to the amino acid sequence of SEQ ID NO:238. In some embodiments, V_{H3} comprises an amino acid sequence that is at least 95%, at least 96%, at least 97%, at least 98%, at least 99%, or 100% identical to the amino acid sequence of SEQ ID NO:239 and V_{L3} comprises an amino acid sequence that is at least 95%, at least 96%, at least 97%, at least 98%, at least 99%, or 100% identical to the amino acid sequence of SEQ ID NO:238. In some embodiments, V_{H3} comprises an amino acid sequence that is at least 95%, at least 96%, at least 97%, at least 98%, at least 99%, or 100% identical to the amino acid sequence of SEQ ID NO:240 and V_{L3} comprises an amino acid sequence that is at least 95%, at least 96%, at least 97%, at least 98%, at least 99%, or 100% identical to the amino acid sequence of SEQ ID NO:241. In some embodiments, the antigen binding site that specifically binds the IL-4 polypeptide comprises a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence selected from the group consisting of SEQ ID NOs:3-5, and a VL domain that comprises a CDR-L1 comprising the amino acid sequence of SEQ ID NO:6 or 7, a CDR-L2 comprising the amino acid sequence of SEQ ID NO:8 or 9, and a CDR-L3 comprising the amino acid sequence of SEQ ID NO:10 or 11. In some embodiments, the antigen binding site that specifically binds the IL-4 polypeptide comprises a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence selected from the group consisting of SEQ ID NOs:24-27, and a VL domain that comprises a CDR-L1 comprising the amino acid sequence of SEQ ID NO:6 or 7, a CDR-L2 comprising the amino acid sequence of SEQ ID NO:8 or 9, and a CDR-L3 comprising the amino acid sequence of SEQ ID NO:10 or 11. In some embodiments, the binding site that specifically binds an IL-4 polypeptide comprises a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3, and a VL domain that comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10. In some embodiments, the binding site that specifically binds an IL-4 polypeptide comprises a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3, and a VL domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11. In some embodiments, the binding site that specifically binds an IL-4 polypeptide comprises a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:4, and a VL domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11. In some embodiments, the binding site that specifically binds an IL-4 polypeptide comprises a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:5, and a VL domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11. In some embodiments, the binding site that specifically binds an IL-4 polypeptide comprises a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24, and a VL domain that comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10. In some embodiments, the binding site that specifically binds an IL-4 polypeptide comprises a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24, and a VL domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11. In some embodiments, the binding site that specifically binds an IL-4 polypeptide comprises a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, and a VL domain that comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10. In some embodiments, the binding site that specifically binds an IL-4 polypeptide comprises a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, and a VL domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11. In some embodiments, the binding site that specifically binds an IL-4 polypeptide comprises a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:26, and a VL domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11. In some embodiments, the binding site that specifically binds an IL-4 polypeptide comprises a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:27, and a VL domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:34 and a VL domain that comprises an amino acid sequence that is at least 95%, at least 96%, at least 97%, at least 98%, at least 99%, or 100% identical to the amino acid sequence of SEQ ID NO:35. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:36 and a VL domain that comprises an amino acid sequence that is at least 95%, at least 96%, at least 97%, at least 98%, at least 99%, or 100% identical to the amino acid sequence of SEQ ID NO:37. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:38 and a VL domain that comprises an amino acid sequence that is at least 95%, at least 96%, at least 97%, at least 98%, at least 99%, or 100% identical to the amino acid sequence of SEQ ID NO:39. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:40 and a VL domain that comprises an amino acid sequence that is at least 95%, at least 96%, at least 97%, at least 98%, at least 99%, or 100% identical to the amino acid sequence of SEQ ID NO:41. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:42 and a VL domain that comprises an amino acid sequence that is at least 95%, at least 96%, at least 97%, at least 98%, at least 99%, or 100% identical to the amino acid sequence of SEQ ID NO:43. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:44 and a VL domain that comprises an amino acid sequence that is at least 95%, at least 96%, at least 97%, at least 98%, at least 99%, or 100% identical to the amino acid sequence of SEQ ID NO:43. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:45 and a VL domain that comprises an amino acid sequence that is at least 95%, at least 96%, at least 97%, at least 98%, at least 99%, or 100% identical to the amino acid sequence of SEQ ID NO:43. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:46 and a VL domain that comprises an amino acid sequence that is at least 95%, at least 96%, at least 97%, at least 98%, at least 99%, or 100% identical to the amino acid sequence of SEQ ID NO:47. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:48 and a VL domain that comprises an amino acid sequence that is at least 95%, at least 96%, at least 97%, at least 98%, at least 99%, or 100% identical to the amino acid sequence of SEQ ID NO:39. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:49 and a VL domain that comprises an amino acid sequence that is at least 95%, at least 96%, at least 97%, at least 98%, at least 99%, or 100% identical to the amino acid sequence of SEQ ID NO:41. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:50 and a VL domain that comprises an amino acid sequence that is at least 95%, at least 96%, at least 97%, at least 98%, at least 99%, or 100% identical to the amino acid sequence of SEQ ID NO:43. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:51 and a VL domain that comprises an amino acid sequence that is at least 95%, at least 96%, at least 97%, at least 98%, at least 99%, or 100% identical to the amino acid sequence of SEQ ID NO:37. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:52 and a VL domain that comprises an amino acid sequence that is at least 95%, at least 96%, at least 97%, at least 98%, at least 99%, or 100% identical to the amino acid sequence of SEQ ID NO:39. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:53 and a VL domain that comprises an amino acid sequence that is at least 95%, at least 96%, at least 97%, at least 98%, at least 99%, or 100% identical to the amino acid sequence of SEQ ID NO:41. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:54 and a VL domain that comprises an amino acid sequence that is at least 95%, at least 96%, at least 97%, at least 98%, at least 99%, or 100% identical to the amino acid sequence of SEQ ID NO:43. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:55 and a VL domain that comprises an amino acid sequence that is at least 95%, at least 96%, at least 97%, at least 98%, at least 99%, or 100% identical to the amino acid sequence of SEQ ID NO:43. In some embodiments, the binding site that specifically binds an IL-4 polypeptide comprises a VH domain that comprises an amino acid sequence that is at least 95%, at least 96%, at least 97%, at least 98%, at least 99%, or 100% identical to the amino acid sequence of SEQ ID NO:56 and a VL domain that comprises an amino acid sequence that is at least 95%, at least 96%, at least 97%, at least 98%, at least 99%, or 100% identical to the amino acid sequence of SEQ ID NO:43. In some embodiments, V_{H1} comprises an amino acid sequence that is at least 95%, at least 96%, at least 97%, at least 98%, at least 99%, or 100% identical to the amino acid sequence of SEQ ID NO:34 and V_{L1} comprises an amino acid sequence that is at least 95%, at least 96%, at least 97%, at least 98%, at least 99%, or 100% identical to the amino acid sequence of SEQ ID NO:35. In some embodiments, V_{H1} comprises an amino acid sequence that is at least 95%, at least 96%, at least 97%, at least 98%, at least 99%, or 100% identical to the amino acid sequence of SEQ ID NO:36 and V_{L1} comprises an amino acid sequence that is at least 95%, at least 96%, at least 97%, at least 98%, at least 99%, or 100% identical to the amino acid sequence of SEQ ID NO:37. In some embodiments, V_{H1} comprises an amino acid sequence that is at least 95%, at least 96%, at least 97%, at least 98%, at least 99%, or 100% identical to the amino acid sequence of SEQ ID NO:38 and V_{L1} comprises an amino acid sequence that is at least 95%, at least 96%, at least 97%, at least 98%, at least 99%, or 100% identical to the amino acid sequence of SEQ ID NO:39. In some embodiments, V_{H1} comprises an amino acid sequence that is at least 95%, at least 96%, at least 97%, at least 98%, at least 99%, or 100% identical to the amino acid sequence of SEQ ID NO:40 and V_{L1} comprises an amino acid sequence that is at least 95%, at least 96%, at least 97%, at least 98%, at least 99%, or 100% identical to the amino acid sequence of SEQ ID NO:41. In some embodiments, V_{H1} comprises an amino acid sequence that is at least 95%, at least 96%, at least 97%, at least 98%, at least 99%, or 100% identical to the amino acid sequence of SEQ ID NO:42 and V_{L1} comprises an amino acid sequence that is at least 95%, at least 96%, at least 97%, at least 98%, at least 99%, or 100% identical to the amino acid sequence of SEQ ID NO:43. In some embodiments, V_{H1} comprises an amino acid sequence that is at least 95%, at least 96%, at least 97%, at least 98%, at least 99%, or 100% identical to the amino acid sequence of SEQ ID NO:44 and V_{L1} comprises an amino acid sequence that is at least 95%, at least 96%, at least 97%, at least 98%, at least 99%, or 100% identical to the amino acid sequence of SEQ ID NO:43. In some embodiments, V_{H1} comprises an amino acid sequence that is at least 95%, at least 96%, at least 97%, at least 98%, at least 99%, or 100% identical to the amino acid sequence of SEQ ID NO:45 and V_{L1} comprises an amino acid sequence that is at least 95%, at least 96%, at least 97%, at least 98%, at least 99%, or 100% identical to the amino acid sequence of SEQ ID NO:43. In some embodiments, V_{H1} comprises an amino acid sequence that is at least 95%, at least 96%, at least 97%, at least 98%, at least 99%, or 100% identical to the amino acid sequence of SEQ ID NO:46 and V_{L1} comprises an amino acid sequence that is at least 95%, at least 96%, at least 97%, at least 98%, at least 99%, or 100% identical to the amino acid sequence of SEQ ID NO:47. In some embodiments, V_{H1} comprises an amino acid sequence that is at least 95%, at least 96%, at least 97%, at least 98%, at least 99%, or 100% identical to the amino acid sequence of SEQ ID NO:48 and V_{L1} comprises an amino acid sequence that is at least 95%, at least 96%, at least 97%, at least 98%, at least 99%, or 100% identical to the amino acid sequence of SEQ ID NO:39. In some embodiments, V_{H1} comprises an amino acid sequence that is at least 95%, at least 96%, at least 97%, at least 98%, at least 99%, or 100% identical to the amino acid sequence of SEQ ID NO:49 and V_{L1} comprises an amino acid sequence that is at least 95%, at least 96%, at least 97%, at least 98%, at least 99%, or 100% identical to the amino acid sequence of SEQ ID NO:41. In some embodiments, V_{H1} comprises an amino acid sequence that is at least 95%, at least 96%, at least 97%, at least 98%, at least 99%, or 100% identical to the amino acid sequence of SEQ ID NO:50 and V_{L1} comprises an amino acid sequence that is at least 95%, at least 96%, at least 97%, at least 98%, at least 99%, or 100% identical to the amino acid sequence of SEQ ID NO:43. In some embodiments, V_{H1} comprises an amino acid sequence that is at least 95%, at least 96%, at least 97%, at least 98%, at least 99%, or 100% identical to the amino acid sequence of SEQ ID NO:51 and V_{L1} comprises an amino acid sequence that is at least 95%, at least 96%, at least 97%, at least 98%, at least 99%, or 100% identical to the amino acid sequence of SEQ ID NO:37. In some embodiments, V_{H1} comprises an amino acid sequence that is at least 95%, at least 96%, at least 97%, at least 98%, at least 99%, or 100% identical to the amino acid sequence of SEQ ID NO:52 and V_{L1} comprises an amino acid sequence that is at least 95%, at least 96%, at least 97%, at least 98%, at least 99%, or 100% identical to the amino acid sequence of SEQ ID NO:39. In some embodiments, V_{H1} comprises an amino acid sequence that is at least 95%, at least 96%, at least 97%, at least 98%, at least 99%, or 100% identical to the amino acid sequence of SEQ ID NO:53 and V_{L1} comprises an amino acid sequence that is at least 95%, at least 96%, at least 97%, at least 98%, at least 99%, or 100% identical to the amino acid sequence of SEQ ID NO:41. In some embodiments, V_{H1} comprises an amino acid sequence that is at least 95%, at least 96%, at least 97%, at least 98%, at least 99%, or 100% identical to the amino acid sequence of SEQ ID NO:54 and V_{L1} comprises an amino acid sequence that is at least 95%, at least 96%, at least 97%, at least 98%, at least 99%, or 100% identical to the amino acid sequence of SEQ ID NO:43. In some embodiments, V_{H1} comprises an amino acid sequence that is at least 95%, at least 96%, at least 97%, at least 98%, at least 99%, or 100% identical to the amino acid sequence of SEQ ID NO:55 and V_{L1} comprises an amino acid sequence that is at least 95%, at least 96%, at least 97%, at least 98%, at least 99%, or 100% identical to the amino acid sequence of SEQ ID NO:43. In some embodiments, V_{H1} comprises an amino acid sequence that is at least 95%, at least 96%, at least 97%, at least 98%, at least 99%, or 100% identical to the amino acid sequence of SEQ ID NO:56 and V_{L1} comprises an amino acid sequence that is at least 95%, at least 96%, at least 97%, at least 98%, at least 99%, or 100% identical to the amino acid sequence of SEQ ID NO:43. In some embodiments, the binding site that specifically binds an IL-13 polypeptide comprises a VH domain that comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59 or 108, and a VL domain that comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62. In some embodiments, the binding site that specifically binds an IL-13 polypeptide comprises a VH domain that comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, and a VL domain that comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62. In some embodiments, the binding site that specifically binds an IL-13 polypeptide comprises a VH domain that comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:108, and a VL domain that comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62. In some embodiments, the binding site that specifically binds an IL-13 polypeptide comprises a VH domain that comprises an amino acid sequence that is at least 95%, at least 96%, at least 97%, at least 98%, at least 99%, or 100% identical to the amino acid sequence of SEQ ID NO:70 and a VL domain that comprises an amino acid sequence that is at least 95%, at least 96%, at least 97%, at least 98%, at least 99%, or 100% identical to the amino acid sequence of SEQ ID NO:71. In some embodiments, the binding site that specifically binds an IL-13 polypeptide comprises a VH domain that comprises an amino acid sequence that is at least 95%, at least 96%, at least 97%, at least 98%, at least 99%, or 100% identical to the amino acid sequence of SEQ ID NO:72 and a VL domain that comprises an amino acid sequence that is at least 95%, at least 96%, at least 97%, at least 98%, at least 99%, or 100% identical to the amino acid sequence of SEQ ID NO:73. In some embodiments, the binding site that specifically binds an IL-13 polypeptide comprises a VH domain that comprises an amino acid sequence that is at least 95%, at least 96%, at least 97%, at least 98%, at least 99%, or 100% identical to the amino acid sequence of SEQ ID NO:74 and a VL domain that comprises an amino acid sequence that is at least 95%, at least 96%, at least 97%, at least 98%, at least 99%, or 100% identical to the amino acid sequence of SEQ ID NO:75. In some embodiments, the binding site that specifically binds an IL-13 polypeptide comprises a VH domain that comprises an amino acid sequence that is at least 95%, at least 96%, at least 97%, at least 98%, at least 99%, or 100% identical to the amino acid sequence of SEQ ID NO:76 and a VL domain that comprises an amino acid sequence that is at least 95%, at least 96%, at least 97%, at least 98%, at least 99%, or 100% identical to the amino acid sequence of SEQ ID NO:77. In some embodiments, V_{H2} comprises an amino acid sequence that is at least 95%, at least 96%, at least 97%, at least 98%, at least 99%, or 100% identical to the amino acid sequence of SEQ ID NO:70 and V_{L2} comprises an amino acid sequence that is at least 95%, at least 96%, at least 97%, at least 98%, at least 99%, or 100% identical to the amino acid sequence of SEQ ID NO:71. In some embodiments, V_{H2} comprises an amino acid sequence that is at least 95%, at least 96%, at least 97%, at least 98%, at least 99%, or 100% identical to the amino acid sequence of SEQ ID NO:72 and V_{L2} comprises an amino acid sequence that is at least 95%, at least 96%, at least 97%, at least 98%, at least 99%, or 100% identical to the amino acid sequence of SEQ ID NO:73. In some embodiments, V_{H2} comprises an amino acid sequence that is at least 95%, at least 96%, at least 97%, at least 98%, at least 99%, or 100% identical to the amino acid sequence of SEQ ID NO:74 and V_{L2} comprises an amino acid sequence that is at least 95%, at least 96%, at least 97%, at least 98%, at least 99%, or 100% identical to the amino acid sequence of SEQ ID NO:75. In some embodiments, V_{H2} comprises an amino acid sequence that is at least 95%, at least 96%, at least 97%, at least 98%, at least 99%, or 100% identical to the amino acid sequence of SEQ ID NO:76 and V_{L2} comprises an amino acid sequence that is at least 95%, at least 96%, at least 97%, at least 98%, at least 99%, or 100% identical to the amino acid sequence of SEQ ID NO:77. In some embodiments, V_{H1} comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, V_{L1} comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11, V_{H2} comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, V_{L2} comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62, V_{H3} comprises a CDR-H1 comprising the amino acid sequence of SEQ ID NO: 199, a CDR-H2 comprising the amino acid sequence of SEQ ID NO:200, and a CDR-H3 comprising the amino acid sequence of SEQ ID NO:201, and V_{L3} comprises a CDR-L1 comprising the amino acid sequence of SEQ ID NO:202, a CDR-L2 comprising the amino acid sequence of PAS, and a CDR-L3 comprising the amino acid sequence of SEQ ID NO:204. In some embodiments, V_{H1} comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3, V_{L1} comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11, V_{H2} comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, V_{L2} comprises a CDR-L₁ comprising the amino acid sequence of SEQ ID NO:60, a CDR-L2 comprising the amino acid sequence of SEQ ID NO:61, and a CDR-L3 comprising the amino acid sequence of SEQ ID NO:62, V_{H3} comprises a CDR-H1 comprising the amino acid sequence of SEQ ID NO: 193, a CDR-H2 comprising the amino acid sequence of SEQ ID NO:194, and a CDR-H3 comprising the amino acid sequence of SEQ ID NO:195, and V_{L3} comprises a CDR-L1 comprising the amino acid sequence of SEQ ID NO:196, a CDR-L2 comprising the amino acid sequence of ATS, and a CDR-L3 comprising the amino acid sequence of SEQ ID NO:198. In some embodiments, V_{H1} comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, V_{L1} comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11, V_{H2} comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, V_{L2} comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62, V_{H3} comprises a CDR-H1 comprising the amino acid sequence of SEQ ID NO:193, a CDR-H2 comprising the amino acid sequence of SEQ ID NO:194, and a CDR-H3 comprising the amino acid sequence of SEQ ID NO:195, and V_{L3} comprises a CDR-L1 comprising the amino acid sequence of SEQ ID NO:196, a CDR-L₂ comprising the amino acid sequence of ATS, and a CDR-L3 comprising the amino acid sequence of SEQ ID NO:198. In some embodiments, V_{H1} comprises the amino acid sequence of SEQ ID NO:53, V_{L1} comprises the amino acid sequence of SEQ ID NO:41, V_{H2} comprises the amino acid sequence of SEQ ID NO:72, V_{L2} comprises the amino acid sequence of SEQ ID NO:73, V_{H3} comprises the amino acid sequence of SEQ ID NO:240, and V_{L3} comprises the amino acid sequence of SEQ ID NO:241. In some embodiments, V_{H1} comprises the amino acid sequence of SEQ ID NO:40, V_{L1} comprises the amino acid sequence of SEQ ID NO:41, V_{H2} comprises the amino acid sequence of SEQ ID NO:72, V_{L2} comprises the amino acid sequence of SEQ ID NO:73, V_{H3} comprises the amino acid sequence of SEQ ID NO:237, and V_{L3} comprises the amino acid sequence of SEQ ID NO:238. In some embodiments, V_{H1} comprises the amino acid sequence of SEQ ID NO:53, V_{L1} comprises the amino acid sequence of SEQ ID NO:41, V_{H2} comprises the amino acid sequence of SEQ ID NO:72, V_{L2} comprises the amino acid sequence of SEQ ID NO:73, V_{H3} comprises the amino acid sequence of SEQ ID NO:239, and V_{L3} comprises the amino acid sequence of SEQ ID NO:238. In some embodiments, the binding protein is capable of inhibiting the function of one or more target proteins. In some embodiments, the binding protein is capable of inhibiting the function of an IL-4 polypeptide and/or an IL-13 polypeptide. In some embodiments, the third antigen binding site specifically binds an NKG2D polypeptide, and wherein the binding protein is capable of inhibiting the function of an NKG2D polypeptide. In some embodiments, at least one of L₁, L₂, L₃ or L₄, is independently 0 amino acids in length. In some embodiments, L₁, L₂, L₃ or L₄ are each independently at least one amino acid in length. In some embodiments, L₁, L₂, L₃ and L₄ each independently are zero amino acids in length or comprise a sequence selected from the group consisting of GGGGSGGGGS (SEQ ID NO:102), GGGGSGGGGSGGGGS (SEQ ID NO:103), S, RT, TKGPS (SEQ ID NO:104), GQPKAAP (SEQ ID NO: 105), and GGSGSSGSGG (SEQ ID NO:106). In some embodiments, L₁, L₂, L₃ and L₄ each independently comprise a sequence selected from the group consisting of GGGGSGGGGS (SEQ ID NO:102), GGGGSGGGGSGGGGS (SEQ ID NO:103), S, RT, TKGPS (SEQ ID NO:104), GQPKAAP (SEQ ID NO: 105), and GGSGSSGSGG (SEQ ID NO:106). In some embodiments, L₁ comprises the sequence GQPKAAP (SEQ ID NO: 105), L₂ comprises the sequence TKGPS (SEQ ID NO:104), L3 comprises the sequence S, and L₄ comprises the sequence RT. In some embodiments, L₁ comprises the sequence GGGGSGGGGS (SEQ ID NO: 102), L₂ comprises the sequence GGGGSGGGGS (SEQ ID NO:102), L₃ is 0 amino acids in length, and L₄ is 0 amino acids in length. In some embodiments, L₁ comprises the sequence GGSGSSGSGG (SEQ ID NO:106), L₂ comprises the sequence GGSGSSGSGG (SEQ ID NO:106), L₃ is 0 amino acids in length, and L₄ is 0 amino acids in length. In some embodiments, L₁ comprises the sequence GGGGSGGGGSGGGGS (SEQ ID NO:103), L₂ is 0 amino acids in length, L₃ comprises the sequence GGGGSGGGGSGGGGS (SEQ ID NO: 103), and L₄ is 0 amino acids in length. In some embodiments, the C_{H3} domain of the second polypeptide chain comprises amino acid substitutions at positions corresponding to positions 354 and 366 of human IgG1 or IgG4 according to EU Index, wherein the amino acid substitutions are S354C and T366W; and wherein the C_{H3} domain of the third polypeptide chain comprises amino acid substitutions at positions corresponding to positions 349, 366, 368, and 407 of human IgG1 or IgG4 according to EU Index, wherein the amino acid substitutions are Y349C, T366S, L368A, and Y407V. In some embodiments, the C_{H3} domain of the second polypeptide chain comprises amino acid substitutions at positions corresponding to positions 349, 366, 368, and 407 of human IgG1 or IgG4 according to EU Index, wherein the amino acid substitutions are Y349C, T366S, L368A, and Y407V; and wherein the C_{H3} domain of the third polypeptide chain comprises amino acid substitutions at positions corresponding to positions 354 and 366 of human IgG1 or IgG4 according to EU Index, wherein the amino acid substitutions are S354C and T366W. In some embodiments, the C_{H3} domains of the second and the third polypeptide chains are human IgG1 or IgG4 C_{H3} domains, and wherein only one of the C_{H3} domains comprises amino acid substitutions at positions corresponding to positions 435 and 436 of human IgG1 or IgG4 according to EU Index, wherein the amino acid substitutions are H435R and Y436F. In some embodiments, the C_{H3} domains of the second and the third polypeptide chains are human IgG1 C_{H3} domains, and wherein the C_{H3} domains each comprise amino acid substitutions at positions corresponding to positions 234 and 235 of human IgG1 according to EU Index, wherein the amino acid substitutions are L234A and L235A. In some embodiments, the C_{H3} domains of the second and the third polypeptide chains are human IgG1 C_{H3} domains, and wherein the C_{H3} domains each comprise amino acid substitutions at positions corresponding to positions 256 and 307 of human IgG1 according to EU Index, wherein the amino acid substitutions are T256D and T307Q. In some embodiments, the first polypeptide chain of the binding protein comprises an amino acid sequence that is at least 95%, at least 96%, at least 97%, at least 98%, at least 99%, or 100% identical to the amino acid sequence of SEQ ID NO:242, the second polypeptide chain of the binding protein comprises an amino acid sequence that is at least 95%, at least 96%, at least 97%, at least 98%, at least 99%, or 100% identical to the amino acid sequence of SEQ ID NO:243, the third polypeptide chain of the binding protein comprises an amino acid sequence that is at least 95%, at least 96%, at least 97%, at least 98%, at least 99%, or 100% identical to the amino acid sequence of SEQ ID NO:244, and the fourth polypeptide chain of the binding protein comprises an amino acid sequence that is at least 95%, at least 96%, at least 97%, at least 98%, at least 99%, or 100% identical to the amino acid sequence of SEQ ID NO:245. In some embodiments, the first polypeptide chain of the binding protein comprises an amino acid sequence that is at least 95%, at least 96%, at least 97%, at least 98%, at least 99%, or 100% identical to the amino acid sequence of SEQ ID NO:246, the second polypeptide chain of the binding protein comprises an amino acid sequence that is at least 95%, at least 96%, at least 97%, at least 98%, at least 99%, or 100% identical to the amino acid sequence of SEQ ID NO:247, the third polypeptide chain of the binding protein comprises an amino acid sequence that is at least 95%, at least 96%, at least 97%, at least 98%, at least 99%, or 100% identical to the amino acid sequence of SEQ ID NO:248, and the fourth polypeptide chain of the binding protein comprises an amino acid sequence that is at least 95%, at least 96%, at least 97%, at least 98%, at least 99%, or 100% identical to the amino acid sequence of SEQ ID NO:249. In some embodiments, the first polypeptide chain of the binding protein comprises an amino acid sequence that is at least 95%, at least 96%, at least 97%, at least 98%, at least 99%, or 100% identical to the amino acid sequence of SEQ ID NO:250, the second polypeptide chain of the binding protein comprises an amino acid sequence that is at least 95%, at least 96%, at least 97%, at least 98%, at least 99%, or 100% identical to the amino acid sequence of SEQ ID NO:251, the third polypeptide chain of the binding protein comprises an amino acid sequence that is at least 95%, at least 96%, at least 97%, at least 98%, at least 99%, or 100% identical to the amino acid sequence of SEQ ID NO:252, and the fourth polypeptide chain of the binding protein comprises an amino acid sequence that is at least 95%, at least 96%, at least 97%, at least 98%, at least 99%, or 100% identical to the amino acid sequence of SEQ ID NO:253. In some embodiments, the first polypeptide chain of the binding protein comprises an amino acid sequence of SEQ ID NO:242, the second polypeptide chain of the binding protein comprises an amino acid sequence of SEQ ID NO:243, the third polypeptide chain of the binding protein comprises an amino acid sequence of SEQ ID NO:244, and the fourth polypeptide chain of the binding protein comprises an amino acid sequence of SEQ ID NO:245. In some embodiments, the first polypeptide chain of the binding protein comprises an amino acid sequence of SEQ ID NO:246, the second polypeptide chain of the binding protein comprises an amino acid sequence of SEQ ID NO:247, the third polypeptide chain of the binding protein comprises an amino acid sequence of SEQ ID NO:248, and the fourth polypeptide chain of the binding protein comprises an amino acid sequence of SEQ ID NO:249. In some embodiments, the first polypeptide chain of the binding protein comprises an amino acid sequence of SEQ ID NO:250, the second polypeptide chain of the binding protein comprises an amino acid sequence of SEQ ID NO:251, the third polypeptide chain of the binding protein comprises an amino acid sequence of SEQ ID NO:252, and the fourth polypeptide chain of the binding protein comprises an amino acid sequence of SEQ ID NO:253.

Further provided herein are nucleic acid molecules (e.g., isolated nucleic acid molecules) comprising a nucleotide sequence encoding the antibody or binding protein of any one of the above embodiments. Further provided herein are vectors (e.g., expression vectors) comprising the nucleic acid molecules of any one of the above embodiments. In some embodiments, the host cell is a mammalian or insect cell. Further provided herein is a kit of polynucleotides, comprising a first polynucleotide that encodes a first polypeptide chain of the binding protein according to any one of the above embodiments, a second polynucleotide that encodes a second polypeptide chain of the binding protein according to any one of the above embodiments, a third polynucleotide that encodes a third polypeptide chain of the binding protein according to any one of the above embodiments, and a fourth polynucleotide that encodes a fourth polypeptide chain of the binding protein according to any one of the above embodiments. Further provided herein are host cells (e.g., isolated host cells) comprising the kit of polynucleotides, nucleic acid molecules, or vector of any one of the above embodiments. Further provided herein are methods of producing a binding protein or antibody, comprising culturing the host cell of any one of the above embodiments such that the binding protein or antibody is produced; wherein optionally the methods further comprise recovering the binding protein or antibody from the host cell.

Further provided herein are antibodies that specifically bind an NKG2D polypeptide, wherein the antibodies comprise a heavy chain variable (VH) domain and a light chain variable (VH) domain. In some embodiments, the VH domain comprises a CDR-H1 comprising the amino acid sequence of SEQ ID NO:157, a CDR-H2 comprising the amino acid sequence of SEQ ID NO:158, and a CDR-H3 comprising the amino acid sequence of SEQ ID NO:159, and the VL domain comprises a CDR-L1 comprising the amino acid sequence of SEQ ID NO:160, a CDR-L2 comprising the amino acid sequence of RDS, and a CDR-L3 comprising the amino acid sequence of SEQ ID NO:162. In some embodiments, the VH domain comprises a CDR-H1 comprising the amino acid sequence of SEQ ID NO:163, a CDR-H2 comprising the amino acid sequence of SEQ ID NO:164, and a CDR-H3 comprising the amino acid sequence of SEQ ID NO:165, and the VL domain comprises a CDR-L1 comprising the amino acid sequence of SEQ ID NO:166, a CDR-L2 comprising the amino acid sequence of YDD, and a CDR-L3 comprising the amino acid sequence of SEQ ID NO:168. In some embodiments, the VH domain comprises a CDR-H1 comprising the amino acid sequence of SEQ ID NO:169, a CDR-H2 comprising the amino acid sequence of SEQ ID NO: 170, and a CDR-H3 comprising the amino acid sequence of SEQ ID NO:171, and the VL domain comprises a CDR-L1 comprising the amino acid sequence of SEQ ID NO:172, a CDR-L2 comprising the amino acid sequence of YDD, and a CDR-L3 comprising the amino acid sequence of SEQ ID NO:174. In some embodiments, the VH domain comprises a CDR-H1 comprising the amino acid sequence of SEQ ID NO: 175, a CDR-H2 comprising the amino acid sequence of SEQ ID NO:176, and a CDR-H3 comprising the amino acid sequence of SEQ ID NO:177, and the VL domain comprises a CDR-L1 comprising the amino acid sequence of SEQ ID NO:178, a CDR-L2 comprising the amino acid sequence of YDD, and a CDR-L3 comprising the amino acid sequence of SEQ ID NO:180. In some embodiments, the VH domain comprises a CDR-H1 comprising the amino acid sequence of SEQ ID NO:181, a CDR-H2 comprising the amino acid sequence of SEQ ID NO: 182, and a CDR-H3 comprising the amino acid sequence of SEQ ID NO:183, and the VL domain comprises a CDR-L1 comprising the amino acid sequence of SEQ ID NO:184, a CDR-L2 comprising the amino acid sequence of QDT, and a CDR-L3 comprising the amino acid sequence of SEQ ID NO:186. In some embodiments, the VH domain comprises a CDR-H1 comprising the amino acid sequence of SEQ ID NO:187, a CDR-H2 comprising the amino acid sequence of SEQ ID NO:188, and a CDR-H3 comprising the amino acid sequence of SEQ ID NO:189, and the VL domain comprises a CDR-L1 comprising the amino acid sequence of SEQ ID NO:190, a CDR-L2 comprising the amino acid sequence of PAS, and a CDR-L3 comprising the amino acid sequence of SEQ ID NO:192. In some embodiments, the VH domain comprises a CDR-H1 comprising the amino acid sequence of SEQ ID NO:193, a CDR-H2 comprising the amino acid sequence of SEQ ID NO:194, and a CDR-H3 comprising the amino acid sequence of SEQ ID NO:195, and the VL domain comprises a CDR-L1 comprising the amino acid sequence of SEQ ID NO:196, a CDR-L2 comprising the amino acid sequence of ATS, and a CDR-L3 comprising the amino acid sequence of SEQ ID NO:198. In some embodiments, the VH domain comprises a CDR-H1 comprising the amino acid sequence of SEQ ID NO:199, a CDR-H2 comprising the amino acid sequence of SEQ ID NO:200, and a CDR-H3 comprising the amino acid sequence of SEQ ID NO:201, and the VL domain comprises a CDR-L1 comprising the amino acid sequence of SEQ ID NO:202, a CDR-L2 comprising the amino acid sequence of PAS, and a CDR-L3 comprising the amino acid sequence of SEQ ID NO:204. In some embodiments, the VH domain comprises a CDR-H1 comprising the amino acid sequence of SEQ ID NO:205, a CDR-H2 comprising the amino acid sequence of SEQ ID NO:206, and a CDR-H3 comprising the amino acid sequence of SEQ ID NO:207, and the VL domain comprises a CDR-L1 comprising the amino acid sequence of SEQ ID NO:208, a CDR-L2 comprising the amino acid sequence of AAS, and a CDR-L3 comprising the amino acid sequence of SEQ ID NO:210. In some embodiments, the VH domain comprises a CDR-H1 comprising the amino acid sequence of SEQ ID NO:211, a CDR-H2 comprising the amino acid sequence of SEQ ID NO:212, and a CDR-H3 comprising the amino acid sequence of SEQ ID NO:213, and the VL domain comprises a CDR-L1 comprising the amino acid sequence of SEQ ID NO:214, a CDR-L2 comprising the amino acid sequence of YAS, and a CDR-L3 comprising the amino acid sequence of SEQ ID NO:216. In some embodiments, the VH domain comprises an amino acid sequence that is at least 95%, at least 96%, at least 97%, at least 98%, at least 99%, or 100% identical to the amino acid sequence of SEQ ID NO:217 and the VL domain comprises an amino acid sequence that is at least 95%, at least 96%, at least 97%, at least 98%, at least 99%, or 100% identical to the amino acid sequence of SEQ ID NO:218. In some embodiments, the VH domain comprises an amino acid sequence that is at least 95%, at least 96%, at least 97%, at least 98%, at least 99%, or 100% identical to the amino acid sequence of SEQ ID NO:219 and the VL domain comprises an amino acid sequence that is at least 95%, at least 96%, at least 97%, at least 98%, at least 99%, or 100% identical to the amino acid sequence of SEQ ID NO:220. In some embodiments, the VH domain comprises an amino acid sequence that is at least 95%, at least 96%, at least 97%, at least 98%, at least 99%, or 100% identical to the amino acid sequence of SEQ ID NO:221 and the VL domain comprises an amino acid sequence that is at least 95%, at least 96%, at least 97%, at least 98%, at least 99%, or 100% identical to the amino acid sequence of SEQ ID NO:222. In some embodiments, the VH domain comprises an amino acid sequence that is at least 95%, at least 96%, at least 97%, at least 98%, at least 99%, or 100% identical to the amino acid sequence of SEQ ID NO:223 and the VL domain comprises an amino acid sequence that is at least 95%, at least 96%, at least 97%, at least 98%, at least 99%, or 100% identical to the amino acid sequence of SEQ ID NO:224. In some embodiments, the VH domain comprises an amino acid sequence that is at least 95%, at least 96%, at least 97%, at least 98%, at least 99%, or 100% identical to the amino acid sequence of SEQ ID NO:225 and the VL domain comprises an amino acid sequence that is at least 95%, at least 96%, at least 97%, at least 98%, at least 99%, or 100% identical to the amino acid sequence of SEQ ID NO:226. In some embodiments, the VH domain comprises an amino acid sequence that is at least 95%, at least 96%, at least 97%, at least 98%, at least 99%, or 100% identical to the amino acid sequence of SEQ ID NO:227 and the VL domain comprises an amino acid sequence that is at least 95%, at least 96%, at least 97%, at least 98%, at least 99%, or 100% identical to the amino acid sequence of SEQ ID NO:228. In some embodiments, the VH domain comprises an amino acid sequence that is at least 95%, at least 96%, at least 97%, at least 98%, at least 99%, or 100% identical to the amino acid sequence of SEQ ID NO:229 and the VL domain comprises an amino acid sequence that is at least 95%, at least 96%, at least 97%, at least 98%, at least 99%, or 100% identical to the amino acid sequence of SEQ ID NO:230. In some embodiments, the VH domain comprises an amino acid sequence that is at least 95%, at least 96%, at least 97%, at least 98%, at least 99%, or 100% identical to the amino acid sequence of SEQ ID NO:231 and the VL domain comprises an amino acid sequence that is at least 95%, at least 96%, at least 97%, at least 98%, at least 99%, or 100% identical to the amino acid sequence of SEQ ID NO:232. In some embodiments, the VH domain comprises an amino acid sequence that is at least 95%, at least 96%, at least 97%, at least 98%, at least 99%, or 100% identical to the amino acid sequence of SEQ ID NO:233 and the VL domain comprises an amino acid sequence that is at least 95%, at least 96%, at least 97%, at least 98%, at least 99%, or 100% identical to the amino acid sequence of SEQ ID NO:234. In some embodiments, the VH domain comprises an amino acid sequence that is at least 95%, at least 96%, at least 97%, at least 98%, at least 99%, or 100% identical to the amino acid sequence of SEQ ID NO:235 and the VL domain comprises an amino acid sequence that is at least 95%, at least 96%, at least 97%, at least 98%, at least 99%, or 100% identical to the amino acid sequence of SEQ ID NO:236. In some embodiments, the VH domain comprises an amino acid sequence that is at least 95%, at least 96%, at least 97%, at least 98%, at least 99%, or 100% identical to the amino acid sequence of SEQ ID NO:237 and the VL domain comprises an amino acid sequence that is at least 95%, at least 96%, at least 97%, at least 98%, at least 99%, or 100% identical to the amino acid sequence of SEQ ID NO:238. In some embodiments, the VH domain comprises an amino acid sequence that is at least 95%, at least 96%, at least 97%, at least 98%, at least 99%, or 100% identical to the amino acid sequence of SEQ ID NO:239 and the VL domain comprises an amino acid sequence that is at least 95%, at least 96%, at least 97%, at least 98%, at least 99%, or 100% identical to the amino acid sequence of SEQ ID NO:238. In some embodiments, the VH domain comprises an amino acid sequence that is at least 95%, at least 96%, at least 97%, at least 98%, at least 99%, or 100% identical to the amino acid sequence of SEQ ID NO:240 and the VL domain comprises an amino acid sequence that is at least 95%, at least 96%, at least 97%, at least 98%, at least 99%, or 100% identical to the amino acid sequence of SEQ ID NO:241. In some embodiments, the antibody is a monospecific antibody. In some embodiments, the antibody is a bispecific or multispecific antibody.

Further provided herein are pharmaceutical compositions comprising the antibody or binding protein of any one of the above embodiments and a pharmaceutically acceptable carrier.

Further provided herein are methods of preventing and/or treating and/or alleviating one or more symptoms of an inflammatory disease or disorder in a patient in need thereof, comprising administering to the patient a therapeutically effective amount of at least one binding protein, antibody, or pharmaceutical composition of any one of the above embodiments. In some embodiments, the inflammatory disease or disorder is atopic dermatitis. In some embodiments, the inflammatory disease or disorder is chronic obstructive pulmonary disease (COPD). In some embodiments, the inflammatory disease or disorder is atopic dermatitis, Crohn's disease, asthma, or chronic obstructive pulmonary disease (COPD). Further provided herein are methods of preventing and/or treating cancer in a patient in need thereof, comprising administering to the patient a therapeutically effective amount of at least one binding protein, antibody, or pharmaceutical composition of any one of the above embodiments. In some embodiments, the patient is a human.

Further provided herein is the binding protein, antibody, or pharmaceutical composition of any one of the above embodiments for use in a method of preventing and/or treating and/or alleviating one or more symptoms of an inflammatory disease or disorder in a patient. In some embodiments, the inflammatory disease or disorder is atopic dermatitis. In some embodiments, the inflammatory disease or disorder is chronic obstructive pulmonary disease (COPD). In some embodiments, the inflammatory disease or disorder is atopic dermatitis, Crohn's disease, asthma, or chronic obstructive pulmonary disease (COPD). In some embodiments, the patient is a human.

Further provided herein is the use of a binding protein, antibody, or pharmaceutical composition of any one of the above embodiments in the manufacture of a medicament for preventing and/or treating and/or alleviating one or more symptoms of an inflammatory disease or disorder in a patient. In some embodiments, the inflammatory disease or disorder is atopic dermatitis. In some embodiments, the inflammatory disease or disorder is chronic obstructive pulmonary disease (COPD). In some embodiments, the inflammatory disease or disorder is atopic dermatitis, Crohn's disease, asthma, or chronic obstructive pulmonary disease (COPD). In some embodiments, the patient is a human.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 shows a schematic of the exemplary bivalent bispecific antibody which includes one cross-over dual variable (CODV) arm targeting IL-4 and IL-13.

FIGS. 2A & 2B show the binding affinity of a molecule comprising a CODV arm with antigen binding domains ADI-46659 (anti-IL-4) and CL-203143 (anti-IL-13) to human and cyno IL-4 and IL-13 (FIG. 2A), compared to a molecule with another molecule comprising comparator antigen binding domains (FIG. 2B). It was found that kD was higher for the molecule with ADI-46659 (anti-IL-4) and CL-203143 (anti-IL-13).

FIG. 3 shows purity and titer of various CODV antibodies as shown in FIG. 1 which comprise different VH/VL domains binding to IL-4 and IL-13. Two antigen binding domains binding to IL-4 (ADI-46653 and ADI-466659) were tested and combined with various antigen binding domains binding IL-13. Both IL-4 antigen binding domains were tested in the outer position (VH most N-terminal on the heavy chain) as well as in the inner position (VH C-terminal from another VH on the heavy chain) position. The left graph indicates the purity as determined by aSEC, the right graph indicates the titer, and the numbers above the columns indicate how many different constructs could be expressed. Surprisingly, the outer position was strongly favored by both ADI-46653 and ADI-466659. The amount of constructs which could be expressed was almost three times as high as for the inner position. Moreover, the titer and the purity were on average better for constructs which had ADI-46653 or ADI-466659 in the outer position.

FIG. 4 shows a schematic of the exemplary trivalent, trispecific antibody C. The antibody includes one cross-over dual variable (CODV) arm targeting IL-4 and IL-13, and one Fab arm targeting OX40L. The backbone is IgG1. Key indicates the domains in which charge mutations (CM1, CM2, CR3, and NN3) and mutations improving properties as measured by Kd-DLS (such as viscosity; V2) are located.

FIG. 5A shows a schematic depicting IL-2 release induced by the interaction between OX40⁺ peripheral blood mononuclear cells (PBMCs) and OX40L⁺ Chinese hamster ovary (CHO) cells.

FIG. 5B shows inhibition of OX40L-induced IL-2 production by PBMCs using trispecific antibody C.

FIG. 6 shows a schematic depicting thymus- and activation-regulated chemokine (TARC) release induced by type I IL-4 receptor (IL-4RI) stimulation mediated by IL-4 binding, and Eotaxin-3 release induced by type II IL-4 receptor (IL-4RII) stimulation mediated by IL-4 or IL-13 binding.

FIGS. 7A-7B show trispecific antibody inhibition of IL-4 (FIG. 7A) and IL-13-mediated (FIG. 7B) TARC release by IL-4RI⁺ PBMCs. Shown are the results for trispecific antibodies B and C and for a comparator bispecific anti-IL-4/IL-13 antibody.

FIGS. 7C-7D show trispecific antibody inhibition of IL-4 and IL-13-mediated Eotaxin-3 release by IL-4RII+A549 epithelial cells. Shown are the results for trispecific antibodies B and C and for a comparator bispecific anti-IL-4/IL-13 antibody.

FIG. 8 shows a schematic depicting exogenous IL-4, IL-13 and allergen Dermatophagoides pteronyssinus (der p) added to tissue, stimulating the release of TARC, IL-5 and Eotaxin-3.

FIGS. 9A-9C show the inhibition of IL-5 (FIG. 9A), TARC (FIG. 9B), and Eotaxin-3 (FIG. 9C) production in an allergen-driven tissue-mimic assay by the indicated trispecific, bispecific, or monospecific antibodies. Shown are the results for trispecific antibody C, for a comparator monospecific anti-OX40L antibody, for a comparator bispecific anti-IL-4/IL-13 antibody, and for a comparator bispecific anti-IL-13/OX40L VHH antibody.

FIG. 10 shows results from a T-cell-dependent antibody response (TDAR) assay induced by the antigen KLH. X-axis (left to right): −KLH, +KLH, 10 mg/kg anti-OX40L Ab, 10 mg/kg trispecific antibody B, 3 mg/kg trispecific antibody B, 1.0 mg/kg trispecific antibody B, 0.3 mg/kg trispecific antibody B. * indicates a p-value less than 0.05. ** indicates a p-value less than 0.01.

FIGS. 11A-11D show eosinophil numbers (FIG. 11A), CD4+ memory cells (FIG. 11B), eotaxin-3 levels (FIG. 11C), and TARC levels (FIG. 11D) in lungs of quintuple knock-in mice upon antigen exposure. Shown are results for treatment with the trispecific antibodies, for a comparator monospecific anti-OX40L antibody, and for a comparator bispecific anti-IL-4/IL-13 antibody.

FIG. 11E shows pathological changes, visualized by Periodic acid-Schiff (PAS) staining, in lungs of quintuple knock-in mice upon antigen exposure. Shown are results for treatment with the trispecific antibodies, for a comparator monospecific anti-OX40L antibody, and for a comparator bispecific anti-IL-4/IL-13 antibody.

FIG. 11F shows pharmacokinetic properties (i.e., concentration over time) of trispecific antibodies in hFcRn Tg32 mice following intravenous injection at a dose of 5 mg/kg.

FIG. 12A shows the isoelectric point data for trispecific antibodies E, B, and C.

FIG. 12B shows the polydispersity of trispecific antibodies E, B, and C as measured by dynamic light scattering (DLS).

FIG. 12C shows kD-DLS values for trispecific antibodies E, B, and C as measured by DLS.

FIG. 12D shows the viscosity of trispecific antibodies E, B, and C.

FIG. 12E shows the turbidity of trispecific antibodies E, B, and C.

FIG. 13 shows the aggregation profiles of trispecific antibodies E, B, and C after three weeks at 40° C. TO represents time 0, T1 represents week 1, T2 represents week 2, and T3 represents week 3.

FIG. 14 shows the structure of the anti-OX40L (MM25PL3A2) Fab: OX40L complex, as determined by CryoEM. Shown are the OX40L trimer bound to 3 MM25PL3A2 Fabs (at a), top view (at b), and side view (at c).

FIG. 15 shows top and bottom views of the CryoEM map of the anti-OX40L (MM25PL3A2) Fab: OX40L complex at a resolution of 3.94 Å.

FIG. 16 depicts the structures of the OX40:OX40L complex (at a), the anti-OX40L (MM25PL3A2) Fab: OX40L complex (at b), and an overlay (at c), showing partial overlap of the Fab and OX40 epitopes on OX40L.

FIG. 17 shows the anti-OX40L (MM25PL3A2) Fab (SEQ ID NO:111) epitope mapped onto the human OX40L amino acid sequence (SEQ ID NO:110; at a) and the anti-OX40L (MM25PL3A2) Fab paratope residues mapped onto the VH (SEQ ID NO:97) and VL (SEQ ID NO:96) domain sequences after energy minimization of the CDR loops fitted in the 3.94A CryoEM map (at b). Sequences in (b) correspond to SEQ ID Nos:112 (upper) and 113 (lower), respectively. For the epitope mapping at a, Fab:OX40L interactions are highlighted as indicated (in the upper OX40L sequence, labelled “MM25”, * or \hat{ } indicating the OX40L monomer the Fab interacts with). Furthermore, the OX40L residues binding to OX40 are shown in gray (in the lower OX40L sequence labelled “OX40”).

FIG. 18A shows a schematic of an exemplary trispecific binding protein with 3 antigen binding sites that specifically bind IL-4, IL-13, and IL-17, respectively. The arm with anti-IL-4 and anti-IL-13 binding sites is configured as a cross-over dual variable (CODV) arm, while the arm with the anti-IL-17 binding site is configured as a traditional Fab arm.

FIG. 18B shows a schematic of an exemplary trispecific binding protein with 3 antigen binding sites that specifically bind IL-4, IL-13, and IL-17, respectively. Positions of mutations to promote association of the two heavy chains (knobs-into-holes or KiH mutations), mutations to promote purification of the assembled binding protein (RF mutation), Fc silencing mutations (LALA), mutations to promote circulation half-life (DQ), and mutations to promote proper chain pairing (CM1, CM2, CR3, and NN3), are depicted.

FIGS. 19A-19E show stability of trispecific binding protein C1 (see Table 18) at pH 5 (circles) or pH 8 (squares) over time, including protein concentration (FIG. 19A), diffusion coefficient (FIG. 19B), % low molecular weight species (left panel in FIG. 19C), % high molecular weight species (right panel in FIG. 19C), size exclusion chromatography (SEC) total area (FIG. 19D), and fragmentation (FIG. 19E).

FIG. 20 shows a diagram of a surface plasmon resonance assay to measure single antigen binding of individual antigen binding domains with a CODV-based trispecific binding protein (as shown in FIG. 18A).

FIGS. 21A-21C show testing of multispecific binding protein effects on IL-4/IL-13/IL-17 activity in vivo; FIG. 21A shows a schematic of a mouse assay for single-dose in vivo inhibition of IL-17; FIG. 21B shows a schematic of a mouse assay for in vivo inhibition of IL-17 in a 7-day imiquimod (IMQ)-induced psoriatic skin inflammation model; FIG. 21C shows suppression of OVA/alum-induced TARC, KC-GRO (CXCL1), and eotaxin in BAL fluid of humanized IL-4/IL-13/IL-4Ra mice by binding protein A1.

FIGS. 22A-22D show in vitro assessment of physicochemical properties of trispecific binding proteins A1, B1, C1, and D1 (see Table 18). Assays included non-specific binding (FIG. 22A), self-association (FIG. 22B), FcRn binding (FIG. 22C), and FcRn cellular recycling (FIG. 22D).

FIGS. 23A-23D show the results of in vivo pharmacokinetic (PK) assessment of binding proteins A1 (FIG. 23A), B1 (FIG. 23B), C1 (FIG. 23C), and D1 (FIG. 23D) following intravenous administration of a 5 mg/kg dose in Tg32 mice. Each set of graphs show the concentration time curve of each tested animal after administration of 5 mg/kg IV of the respective binding protein.

FIGS. 24A & 24B show the results of testing trispecific binding proteins (BP) A1, B1, C1, and D1 for stability during incubation for 3 weeks in various buffers ranging from pH 5.2-7.2 (as labeled). In FIG. 24A, percentage of high molecular weight species (HMWS) was quantified using SEC at 0, 1, 2, or 3 weeks. In FIG. 24B, percentage of low molecular weight species (LMWS) was quantified using SEC at 0, 1, 2, or 3 weeks.

FIG. 25 shows the results of testing trispecific binding proteins B1, C1, and D1 for isomerization of residue D30 in the anti-IL-17 binding domain during incubation for 3 weeks in various buffers ranging from pH 5.2-7.2. In each graph, % of isoaspartic acid is shown at each of the time points (TO-T3, left to right). Each group of 4 data points represents pH 5.2 buffer, pH 6.0 buffer A, pH 6.0 buffer B, and pH 7.2 buffer (left to right).

FIGS. 26A-26D show the results of structural mapping of anti-IL-17 Fab:human IL-17A interaction by x-ray crystallography; FIG. 26A shows a structure of two anti-IL-17 Fab molecules interacting with a single human IL-17A homodimer; FIG. 26B shows the epitope and paratope of the anti-IL-17 Fab:human IL-17A interaction (residues within a 4A distance between human IL-17A chain A or B and the anti-IL-17 Fab are indicated); FIG. 26C highlights the residues of the IL-17 epitope bound by the anti-IL-17 Fab (sequence of human IL-17A as depicted corresponds to SEQ ID NO:156); FIG. 26D highlights the residues of the anti-IL-17 Fab paratope that interact with human IL-17 (heavy chain sequence of anti-IL-17 Fab as depicted corresponds to SEQ ID NO:154; light chain sequence of anti-IL-17 Fab as depicted corresponds to SEQ ID NO:155).

FIG. 27A shows a schematic of an exemplary trispecific binding protein with 3 antigen binding sites that specifically bind IL-4, IL-13, and NKG2D, respectively. The arm with anti-IL-4 and anti-IL-13 binding sites is configured as a cross-over dual variable (CODV) arm, while the arm with the anti-NKG2D binding site is configured as a traditional Fab arm.

FIG. 27B shows a schematic of an exemplary trispecific binding protein with 3 antigen binding sites that specifically bind IL-4, IL-13, and NKG2D, respectively. Positions of mutations to promote association of the two heavy chains (knobs-into-holes or KiH mutations), mutations to promote purification of the assembled binding protein (RF mutation), mutations to promote proper chain pairing (CM1, CM2, CR3, and NN3), mutations to reduce effector function (LALA), and mutations to extend half-life (HLE) are depicted.

FIG. 28A details the characterization of the top 10 anti-IL4/IL-13/NKG2D trispecific binding proteins. Antigen binding domains for each of the three domains for each molecule are as indicated. SEC: size exclusion chromatography; HIC: hydrophobic interaction chromatography; DSF: differential scanning fluorimetry; cIEF: capillary isoelectric focusing.

FIG. 28B details the binding of the top 10 anti-IL4/IL-13/NKG2D trispecific binding proteins to all three targets. cIL13: cynomolgus IL-13; hIL13: human IL-13; cIL4: cynomolgus IL-4; hIL4: human IL-4; cNKG2D: cynomolgus NKG2D; hNKG2D: human NKG2D; DSF: differential scanning fluorimetry; SPR: surface plasmon resonance.

FIGS. 29A-29C show crystal structures of anti-NKG2D Fab complexes. FIG. 29A: CL-523781 Fab in complex with NKG2D dimer at a resolution of 2.6 Å and refined to an R and Rfree of 19 and 25% respectively. FIG. 29B: CL-526201 Fab in complex with NKG2D dimer at a resolution of 2.8 Å and refined to R and Rfre of 20 and 27% respectively. FIG. 29C: CL-524785 Fab in complex with NKG2D dimer at a resolution of 2.9 Å and refined to R and Rfee of 23 and 30% respectively.

FIG. 29D shows that the CL-523781 and NKG2D ligand ULBP6 epitopes overlap partially. However, binding of two Fabs to NKG2D dimer would sterically hinder the binding of natural ligands to NKG2D.

FIG. 29E shows the respective epitopes of CL-523781, CL-526201, CL-524785 mapped onto the NKG2D dimer. The epitope residues engaged with Fab light chain and Fab heavy chain are indicated by \hat{ } and *, respectively. The epitope residues interacting with both Fab heavy and light chain paratope residues indicated by “!”. Amino acid sequence corresponds to SEQ ID NO:255.

FIG. 29F shows the paratopes of CL-523781 (SEQ ID Nos:227 and 228 for the VH and VL domain sequences, respectively), CL-526201 (SEQ ID Nos:231 and 232 for the VH and VL domain sequences, respectively), and CL-524785 (SEQ ID Nos:229 and 230 for the VH and VL domain sequences, respectively) for binding human NKG2D. The CDR loops according to IMGT classification are underlined and the paratope residues are highlighted in yellow. Amino acid sequences correspond to SEQ ID Nos:256 and 257 for heavy and light chains of CL-523781 (respectively), SEQ ID Nos:258 and 259 for heavy and light chains of CL-526201 (respectively), and SEQ ID Nos:260 and 261 for heavy and light chains of CL-524785 (respectively).

FIG. 29G shows the results of modeling the interaction between anti-NKG2D antigen binding domains CL-526201 or CL-523781 bound to either NKG2D homodimer or MICA/MICB.

FIGS. 30A-30E show the results of in vitro pharmacology assays demonstrating that anti-IL-4/IL-13/NKG2D trispecific binding proteins exhibited good potency on inhibiting NKG2D, IL-4, and IL-13 target functions.

FIG. 31 summarizes production of three trispecific binding proteins: A2 ((a-IL4_VR-62824 x a-IL13_VR-62822) x a-KLRK1_VR-93305 huIgG1_LALA_DQ CR3 NN3_dK), B2 ((a-IL4_VR-93303 x a-IL13_VR-62822) x a-KLRK1_VR-91186_huIgG1_LALA_DQ CR3 NN3_dK), and C2 ((a-IL4_VR-82824 x a-IL13_VR-62822) x a-KLRK1_VR-93304_huIgG1_LALA_DQ_CR3_NN3_dK).

FIGS. 32A-32C show stability of trispecific binding proteins A2 (FIG. 32A), B2 (FIG. 32B), and C2 (FIG. 32C) over 3 weeks at pH 5 (*) or pH 8 (\hat{ }).

### DETAILED DESCRIPTION

The disclosure relates to trispecific binding proteins comprising four polypeptide chains that form three antigen binding sites that specifically bind one or more target proteins, wherein a first pair of polypeptides forming the binding protein possess dual variable domains having a cross-over orientation, and at least two of the antigen binding sites specifically bind an interleukin-4 (IL-4) polypeptide and an interleukin-13 (IL-13) polypeptide, respectively. In some embodiments, the third antigen binding site specifically binds an OX40L polypeptide, and IL-17 polypeptide, or an NKG2D polypeptide. The disclosure also provides monospecific and multispecific binding proteins that specifically bind an IL-4, IL-13, OX40L, IL-17, or NKG2D polypeptide. The disclosure also relates to nucleic acid molecules, expression vectors, host cells, compositions (e.g., pharmaceutical compositions), methods of production, methods of use, and uses related thereto.

### General Definitions

As utilized in accordance with the present disclosure, the following terms, unless otherwise indicated, shall be understood to have the following meanings. Unless otherwise required by context, singular terms shall include pluralities and plural terms shall include the singular.

It is understood that aspects and embodiments of the disclosure described herein include “comprising,” “consisting,” and “consisting essentially of” aspects and embodiments.

The term “polynucleotide” as used herein refers to single-stranded or double-stranded nucleic acid polymers of at least 10 nucleotides in length. In certain embodiments, the nucleotides comprising the polynucleotide can be ribonucleotides or deoxyribonucleotides or a modified form of either type of nucleotide. Such modifications include base modifications such as bromuridine, ribose modifications such as arabinoside and 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single-stranded and double-stranded forms of DNA.

An “isolated polynucleotide” is a polynucleotide of genomic, cDNA, or synthetic origin or some combination thereof, which: (1) is not associated with all or a portion of a polynucleotide in which the isolated polynucleotide is found in nature, (2) is linked to a polynucleotide to which it is not linked in nature, or (3) does not occur in nature as part of a larger sequence.

An “isolated polypeptide” is one that: (1) is free of at least some other polypeptides with which it would normally be found, (2) is essentially free of other polypeptides from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or noncovalent interaction) with portions of a polypeptide with which the “isolated polypeptide” is associated in nature, (6) is operably associated (by covalent or noncovalent interaction) with a polypeptide with which it is not associated in nature, or (7) does not occur in nature. Such an isolated polypeptide can be encoded by genomic DNA, cDNA, mRNA or other RNA, of synthetic origin, or any combination thereof. Preferably, the isolated polypeptide is substantially free from polypeptides or other contaminants that are found in its natural environment that would interfere with its use (therapeutic, diagnostic, prophylactic, research or otherwise).

Naturally occurring antibodies typically comprise a tetramer. Each such tetramer is typically composed of two identical pairs of polypeptide chains, each pair having one full-length “light” chain (typically having a molecular weight of about 25 kDa) and one full-length “heavy” chain (typically having a molecular weight of about 50-70 kDa). The terms “heavy chain” and “light chain” as used herein refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The carboxy-terminal portion of each chain typically defines a constant domain responsible for effector function. Thus, in a naturally occurring antibody, a full-length heavy chain immunoglobulin polypeptide includes a variable domain (VH) and three constant domains (C_{H1}, C_{H2}, and C_{H3}), wherein the VH domain is at the amino-terminus of the polypeptide and the C_{H3} domain is at the carboxyl-terminus, and a full-length light chain immunoglobulin polypeptide includes a variable domain (V_{L}) and a constant domain (C_{L}), wherein the VL domain is at the amino-terminus of the polypeptide and the C_{L} domain is at the carboxyl-terminus.

Human light chains are typically classified as kappa and lambda light chains, and human heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, the variable and constant domains typically are joined by a “J” region of about 12 or more amino acids, with the heavy chain also including a “D” region of about 10 more amino acids. See, e.g., FUNDAMENTAL IMMUNOLOGY (Paul, W., ed., Raven Press, 2nd ed., 1989), which is incorporated by reference in its entirety for all purposes. The variable regions of each light/heavy chain pair typically form an antigen binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

The term “CDR set” refers to a group of three CDRs that occur in a single variable region capable of binding the antigen. The exact boundaries of these CDRs have been defined differently according to different systems. The system described by Kabat (Kabat et al., SEQUENCES OF PROTEINS OF IMMUNOLOGICAL INTEREST (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia and Lesk, 1987, J. Mol. Biol. 196: 901-17; Chothia et al., 1989, Nature 342: 877-83) found that certain sub-portions within Kabat CDRs adopt nearly identical peptide backbone conformations, despite having great diversity at the level of amino acid sequence. These sub-portions were designated as L1, L2, and L3 or H1, H2, and H3 where the “L” and the “H” designates the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan, 1995, FASEB J. 9: 133-39; MacCallum, 1996, J Mol. Biol. 262(5): 732-45; and Lefranc, 2003, Dev. Comp. Immunol. 27: 55-77. Another system for defining CDRs is the IMGT definition. (see, e.g., imgt.org/IMGTScientificChart/Nomenclature/IMGT-FRCDRdefinition.html). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or IMGT defined CDRs. Identification of predicted CDRs using the amino acid sequence is well known in the field, such as in Martin, A.C. “Protein sequence and structure analysis of antibody variable domains,” In Antibody Engineering, Vol. 2. Kontermann R., Dubel S., eds. Springer-Verlag, Berlin, p. 33-51 (2010). The amino acid sequence of the heavy and/or light chain variable domain may be also inspected to identify the sequences of the CDRs by other conventional methods, e.g., by comparison to known amino acid sequences of other heavy and light chain variable regions to determine the regions of sequence hypervariability. The numbered sequences may be aligned by eye, or by employing an alignment program such as one of the CLUSTAL suite of programs, as described in Thompson, 1994, Nucleic Acids Res. 22: 4673-80. Molecular models are conventionally used to correctly delineate framework and CDR regions and thus correct the sequence-based assignments.

The term “Fc” as used herein refers to a molecule comprising the sequence of a non-antigen-binding fragment resulting from digestion of an antibody or produced by other means, whether in monomeric or multimeric form, and can contain the hinge region. The original immunoglobulin source of the native Fc is preferably of human origin and can be any of the immunoglobulins, although IgG1 and IgG2 are preferred. Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgA1, and IgGA2). One example of a Fc is a disulfide-bonded dimer resulting from papain digestion of an IgG. The term “native Fc” as used herein is generic to the monomeric, dimeric, and multimeric forms.

A F(ab) fragment typically includes one light chain and the VH and C_{H1} domains of one heavy chain, wherein the V_{H}-C_{H1} heavy chain portion of the F(ab) fragment cannot form a disulfide bond with another heavy chain polypeptide. As used herein, a F(ab) fragment can also include one light chain containing two variable domains separated by an amino acid linker and one heavy chain containing two variable domains separated by an amino acid linker and a C_{H1} domain.

A F(ab′) fragment typically includes one light chain and a portion of one heavy chain that contains more of the constant region (between the C_{H1} and C_{H2} domains), such that an interchain disulfide bond can be formed between two heavy chains to form a F(ab′)₂ molecule.

The term “binding protein” as used herein refers to a non-naturally occurring (or recombinant or engineered) molecule that specifically binds to at least one target antigen. A trispecific binding protein of the present disclosure may comprise four polypeptide chains that form at least three antigen binding sites, wherein a first polypeptide chain has a structure represented by the formula:

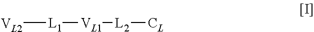

and a second polypeptide chain has a structure represented by the formula:

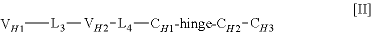

and a third polypeptide chain has a structure represented by the formula:

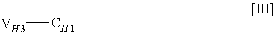

and a fourth polypeptide chain has a structure represented by the formula:

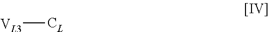

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is the immunoglobulin C_{H1} heavy chain constant domain; and
hinge is an immunoglobulin hinge region connecting the C_{H1} and C_{H2} domains;
L₁, L₂, L₃ and L₄ are amino acid linkers;
and wherein the polypeptide of formula I and the polypeptide of formula II form a cross-over light chain-heavy chain pair.

A “recombinant” molecule is one that has been prepared, expressed, created, or isolated by recombinant means.

One embodiment of the disclosure provides binding proteins having biological and immunological specificity to three target antigens. Another embodiment of the disclosure provides nucleic acid molecules comprising nucleotide sequences encoding polypeptide chains that form such binding proteins. Another embodiment of the disclosure provides expression vectors comprising nucleic acid molecules comprising nucleotide sequences encoding polypeptide chains that form such binding proteins. Yet another embodiment of the disclosure provides host cells that express such binding proteins (i.e., comprising nucleic acid molecules or vectors encoding polypeptide chains that form such binding proteins).

The term “swapability” as used herein refers to the interchangeability of variable domains within the binding protein format and with retention of folding and ultimate binding affinity. “Full swapability” refers to the ability to swap the order of both V_{H1} and V_{H2} domains, and therefore the order of V_{L1} and V_{L2} domains, in the polypeptide chain of formula I or the polypeptide chain of formula II (i.e., to reverse the order) while maintaining full functionality of the binding protein as evidenced by the retention of binding affinity. Furthermore, it should be noted that the designations V_{H} and V_{L} refer only to the domain's location on a particular protein chain in the final format. For example, V_{H1} and V_{H2} could be derived from V_{L1} and V_{L2} domains in parent antibodies and placed into the V_{H1} and V_{H2} positions in the binding protein. Likewise, V_{L1} and V_{L2} could be derived from V_{H1} and V_{H2} domains in parent antibodies and placed in the V_{H1} and V_{H2} positions in the binding protein. Thus, the V_{H} and V_{L} designations refer to the present location and not the original location in a parent antibody. V_{H} and V_{L} domains are therefore “swappable.”

The term “antigen” or “target antigen” or “antigen target” as used herein refers to a molecule or a portion of a molecule that is capable of being bound by a binding protein. A target antigen may have one or more epitopes.

The term “trispecific binding protein” refers to a binding protein that specifically binds to three different antigen targets.

The term “trivalent binding protein” refers to a binding protein that has three binding sites. In particular embodiments the trivalent binding protein can bind to one antigen target. In other embodiments, the trivalent binding protein can bind to two antigen targets. In other embodiments, the trivalent binding protein can bind to three antigen targets.

An “isolated” binding protein is one that has been identified and separated and/or recovered from a component of its natural environment. Contaminant components of its natural environment are materials that would interfere with diagnostic or therapeutic uses for the binding protein, and may include enzymes, hormones, and other proteinaceous or non-proteinaceous solutes. In some embodiments, the binding protein will be purified: (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated binding proteins include the binding protein in situ within recombinant cells since at least one component of the binding protein's natural environment will not be present.

The terms “substantially pure” or “substantially purified” as used herein refer to a compound or species that is the predominant species present (i.e., on a molar basis it is more abundant than any other individual species in the composition). In some embodiments, a substantially purified fraction is a composition wherein the species comprises at least about 50% (on a molar basis) of all macromolecular species present. In other embodiments, a substantially pure composition will comprise more than about 80%, 85%, 90%, 95%, or 99% of all macromolar species present in the composition. In still other embodiments, the species is purified to essential homogeneity (contaminant species cannot be detected in the composition by conventional detection methods) wherein the composition consists essentially of a single macromolecular species.

The term “epitope” includes any determinant, preferably a polypeptide determinant, capable of specifically binding to an immunoglobulin or T-cell receptor. In certain embodiments, epitope determinants include chemically active surface groupings of molecules such as amino acids, sugar side chains, phosphoryl groups, or sulfonyl groups, and, in certain embodiments, may have specific three-dimensional structural characteristics and/or specific charge characteristics. An epitope is a region of an antigen that is bound by an antibody or binding protein. In certain embodiments, a binding protein is said to specifically bind an antigen when it preferentially recognizes its target antigen in a complex mixture of proteins and/or macromolecules. In some embodiments, a binding protein is said to specifically bind an antigen when the equilibrium dissociation constant is ≤10⁻⁸ M, more preferably when the equilibrium dissociation constant is ≤10⁹ M, and most preferably when the dissociation constant is ≤10⁻¹⁰ M.

The dissociation constant (K_{D}) of a binding protein can be determined, for example, by surface plasmon resonance. Generally, surface plasmon resonance analysis measures real-time binding interactions between ligand (a target antigen on a biosensor matrix) and analyte (a binding protein in solution) by surface plasmon resonance (SPR) using the BIAcore system (Pharmacia Biosensor; Piscataway, NJ). Surface plasmon analysis can also be performed by immobilizing the analyte (binding protein on a biosensor matrix) and presenting the ligand (target antigen). The term “K,” as used herein refers to the dissociation constant of the interaction between a particular binding protein and a target antigen.

The term “specifically binds” as used herein refers to the ability of a binding protein or an antigen-binding fragment thereof to bind to an antigen containing an epitope with an Kd of at least about 1×10⁻⁶ M, 1×10⁻⁷ M, 1×10⁻⁸ M, 1×10⁻⁹ M, 1×10⁻¹⁰ M, 1×10⁻¹¹ M, 1 x 10⁻¹² M, or more, and/or to bind to an epitope with an affinity that is at least two-fold greater than its affinity for a nonspecific antigen.

The term “linker” as used herein refers to one or more amino acid residues inserted between immunoglobulin domains to provide sufficient mobility for the domains of the light and heavy chains to fold into cross over dual variable region immunoglobulins. A linker is inserted at the transition between variable domains or between variable and constant domains, respectively, at the sequence level. The transition between domains can be identified because the approximate size of the immunoglobulin domains are well understood. The precise location of a domain transition can be determined by locating peptide stretches that do not form secondary structural elements such as beta-sheets or alpha-helices as demonstrated by experimental data or as can be assumed by techniques of modeling or secondary structure prediction. The linkers described herein are referred to as L₁, which is located on the light chain between the C-terminus of the V_{L2} and the N-terminus of the V_{L1} domain; and L₂, which is located on the light chain between the C-terminus of the V_{L1} and the N-terminus of the C_{L} domain. The heavy chain linkers are known as L₃, which is located between the C-terminus of the V_{H1} and the N-terminus of the V_{H2} domain; and L₄, which is located between the C-terminus of the V_{H2} and the N-terminus of the C_{H1} domain.

The term “vector” as used herein refers to any molecule (e.g., nucleic acid, plasmid, or virus) that is used to transfer coding information to a host cell. The term “vector” includes a nucleic acid molecule that is capable of transporting another nucleic acid to which it has been linked. One type of vector is a “plasmid,” which refers to a circular double-stranded DNA molecule into which additional DNA segments may be inserted. Another type of vector is a viral vector, wherein additional DNA segments may be inserted into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell and thereby are replicated along with the host genome. In addition, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. The terms “plasmid” and “vector” may be used interchangeably herein, as a plasmid is the most commonly used form of vector. However, the disclosure is intended to include other forms of expression vectors, such as viral vectors (e.g., replication defective retroviruses, adenoviruses, and adeno-associated viruses), which serve equivalent functions.

The phrase “recombinant host cell” (or “host cell”) as used herein refers to a cell into which a recombinant expression vector has been introduced. A recombinant host cell or host cell is intended to refer not only to the particular subject cell, but also to the progeny of such a cell. Because certain modifications may occur in succeeding generations due to either mutation or environmental influences, such progeny may not, in fact, be identical to the parent cell, but such cells are still included within the scope of the term “host cell” as used herein. A wide variety of host cell expression systems can be used to express the binding proteins, including bacterial, yeast, baculoviral, and mammalian expression systems (as well as phage display expression systems). An example of a suitable bacterial expression vector is pUC19. To express a binding protein recombinantly, a host cell is transformed or transfected with one or more recombinant expression vectors carrying DNA fragments encoding the polypeptide chains of the binding protein such that the polypeptide chains are expressed in the host cell and, preferably, secreted into the medium in which the host cells are cultured, from which medium the binding protein can be recovered.

The term “transformation” as used herein refers to a change in a cell's genetic characteristics, and a cell has been transformed when it has been modified to contain a new DNA. For example, a cell is transformed where it is genetically modified from its native state. Following transformation, the transforming DNA may recombine with that of the cell by physically integrating into a chromosome of the cell, or may be maintained transiently as an episomal element without being replicated, or may replicate independently as a plasmid. A cell is considered to have been stably transformed when the DNA is replicated with the division of the cell. The term “transfection” as used herein refers to the uptake of foreign or exogenous DNA by a cell, and a cell has been “transfected” when the exogenous DNA has been introduced inside the cell membrane. A number of transfection techniques are well known in the art. Such techniques can be used to introduce one or more exogenous DNA molecules into suitable host cells.

The term “naturally occurring” as used herein and applied to an object refers to the fact that the object can be found in nature and has not been manipulated by man. For example, a polynucleotide or polypeptide that is present in an organism (including viruses) that can be isolated from a source in nature and that has not been intentionally modified by man is naturally-occurring. Similarly, “non-naturally occurring” as used herein refers to an object that is not found in nature or that has been structurally modified or synthesized by man.

As used herein, the twenty conventional amino acids and their abbreviations follow conventional usage. Stereoisomers (e.g., D-amino acids) of the twenty conventional amino acids; unnatural amino acids and analogs such as α-, α-disubstituted amino acids, N-alkyl amino acids, lactic acid, and other unconventional amino acids may also be suitable components for the polypeptide chains of the binding proteins. Examples of unconventional amino acids include: 4-hydroxyproline, γ-carboxyglutamate, ε-N,N,N-trimethyllysine, ε-N-acetyllysine, 0-phosphoserine, N-acetylserine, N-formylmethionine, 3-methylhistidine, 5-hydroxylysine, G-N-methylarginine, and other similar amino acids and imino acids (e.g., 4-hydroxyproline). In the polypeptide notation used herein, the left-hand direction is the amino terminal direction and the right-hand direction is the carboxyl-terminal direction, in accordance with standard usage and convention.

Naturally occurring residues may be divided into classes based on common side chain properties:
(1) hydrophobic: Met, Ala, Val, Leu, Ile, Phe, Trp, Tyr, Pro;
(2) polar hydrophilic: Arg, Asn, Asp, Gln, Glu, His, Lys, Ser, Thr;
(3) aliphatic: Ala, Gly, Ile, Leu, Val, Pro;
(4) aliphatic hydrophobic: Ala, Ile, Leu, Val, Pro;
(5) neutral hydrophilic: Cys, Ser, Thr, Asn, Gln;
(6) acidic: Asp, Glu;
(7) basic: His, Lys, Arg;
(8) residues that influence chain orientation: Gly, Pro;
(9) aromatic: His, Trp, Tyr, Phe; and
(10) aromatic hydrophobic: Phe, Trp, Tyr.

Conservative amino acid substitutions may involve exchange of a member of one of these classes with another member of the same class. Non-conservative substitutions may involve the exchange of a member of one of these classes for a member from another class.

The term “patient” as used herein includes human and animal subjects.

The terms “treatment” or “treat” as used herein refer to therapeutic treatment and/or alleviating symptoms, e.g., in those in need thereof. Those in need of treatment include those having a disorder. In particular embodiments, binding proteins can be used to treat humans with a cancer or an inflammatory disease or disorder, or ameliorate a cancer or an inflammatory disease or disorder in a human subject. In particular embodiments, the inflammatory disease or disorder is atopic dermatitis.

The terms “prevention” or “prevent” as used herein refer to prophylactic or preventative measures. Those in need of prevention include those prone to have the disorder or those in which the disorder is to be prevented. In particular embodiments, binding proteins can be used to prevent humans prone to have a cancer or an inflammatory disease or disorder, or humans susceptible to a cancer or an inflammatory disease or disorder. The binding proteins can also be used to prevent a cancer or an inflammatory disease or disorder in a human patient. In particular embodiments, the inflammatory disease or disorder is atopic dermatitis.

The terms “pharmaceutical composition” or “therapeutic composition” as used herein refer to a compound or composition capable of inducing a desired therapeutic effect when properly administered to a patient.

The term “pharmaceutically acceptable carrier” or “physiologically acceptable carrier” as used herein refers to one or more formulation materials suitable for accomplishing or enhancing the delivery of a binding protein.

The terms “effective amount” and “therapeutically effective amount” when used in reference to a pharmaceutical composition comprising one or more binding proteins refer to an amount or dosage sufficient to produce a desired therapeutic result. More specifically, a therapeutically effective amount may be an amount of a binding protein sufficient to inhibit, for some period of time, one or more of the clinically defined pathological processes associated with the condition being treated. The effective amount may vary depending on the specific binding protein that is being used, and also depends on a variety of factors and conditions related to the patient being treated and the severity of the disorder. For example, if the binding protein is to be administered in vivo, factors such as the age, weight, and health of the patient as well as dose response curves and toxicity data obtained in preclinical animal work may be among those factors considered.

One embodiment of the disclosure provides a pharmaceutical composition comprising a pharmaceutically acceptable carrier and a therapeutically effective amount of a binding protein.

### Trispecific Binding Proteins

Certain aspects of the present disclosure relate to trispecific and/or trivalent binding proteins comprising four polypeptide chains that form three antigen binding sites that specifically bind to one or more target proteins, wherein a first pair of polypeptides forming the binding protein possess dual variable domains having a cross-over orientation (e.g., a CODV arm) and wherein a second pair of polypeptides forming the binding protein possess a single variable domain (e.g., a Fab arm). Any of the CDRs or variable domains of any of the antigen binding proteins described herein may find use in a trispecific and/or trivalent binding protein of the present disclosure.

In some embodiments, each of the three antigen binding sites binds a different target (e.g., polypeptide antigen). In some embodiments, at least one of the three antigen binding sites specifically binds an interleukin-17 (IL-17) polypeptide. In some embodiments, at least one of the three antigen binding sites specifically binds an OX40L polypeptide. In some embodiments, at least one of the three antigen binding sites specifically binds an expressed on the surface of a natural killer (NK) cell, e.g., a natural killer group 2D (NKG2D) polypeptide. In some embodiments, the antigen is a tumor necrosis factor receptor superfamily (TNFRSF) polypeptide.

In some embodiments, the trispecific binding protein comprises four polypeptide chains that form the three antigen binding sites, wherein a first polypeptide chain comprises a structure represented by the formula:

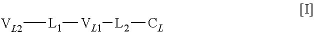

and a second polypeptide chain comprises a structure represented by the formula:

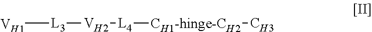

and a third polypeptid chain comprises a structure represented by the formula:

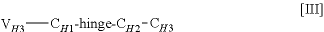

and a fourth polypeptide chain comprises a structure represented by the formula:

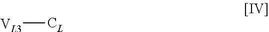

wherein:
   V_{L1} is a first immunoglobulin light chain variable domain;
   V_{L2} is a second immunoglobulin light chain variable domain;
   V_{L3} is a third immunoglobulin light chain variable domain;
   V_{H1} is a first immunoglobulin heavy chain variable domain;
   V_{H2} is a second immunoglobulin heavy chain variable domain;
   V_{H3} is a third immunoglobulin heavy chain variable domain;
   C_{L} is an immunoglobulin light chain constant domain;
   C_{H1} is an immunoglobulin C_{H1} heavy chain constant domain;
   C_{H2} is an immunoglobulin C_{H2} heavy chain constant domain;
   C_{H3} is an immunoglobulin C_{H3} heavy chain constant domain;
   hinge is an immunoglobulin hinge region connecting the C_{H1} and C_{H2} domains; and
   L₁, L₂, L₃ and L₄ are amino acid linkers;
wherein the polypeptide of formula I and the polypeptide of formula II form a cross-over light chain-heavy chain pair.

In some embodiments, a trispecific binding protein of the present disclosure comprises a V_{H1} and V_{L1} domain pair that form a first antigen binding site, a V_{H2} and V_{L2} domain pair that form a second antigen binding site, and a V_{H3} and V_{L3} domain pair that form a third antigen binding site. It is contemplated that any of the antigen binding sites described herein may find use in a trispecific binding protein of the present disclosure, e.g., comprising four polypeptide chains having the structures described supra.

In some embodiments, a trispecific binding protein of the present disclosure comprises a V_{H1} and V_{L1} domain pair that form a first antigen binding site that specifically binds an IL-4 polypeptide, a V_{H2} and V_{L2} domain pair that form a second antigen binding site that specifically binds an IL-13 polypeptide, and a V_{H3} and V_{L3} domain pair that form a third antigen binding site that specifically binds a third target antigen. In some embodiments, the third target antigen is not an IL-4 polypeptide or an IL-13 polypeptide. In some embodiments, a trispecific binding protein of the present disclosure comprises a V_{H1} and V_{L1} domain pair that form a first antigen binding site that specifically binds an IL-4 polypeptide, a V_{H2} and V_{L2} domain pair that form a second antigen binding site that specifically binds an IL-13 polypeptide, and a V_{H3} and V_{L3} domain pair that form a third antigen binding site that specifically binds an OX40L polypeptide.

In some embodiments, a trispecific binding protein of the present disclosure comprises a V_{H1} and V_{L1} domain pair that form a first antigen binding site that specifically binds an IL-13 polypeptide, a V_{H2} and V_{L2} domain pair that form a second antigen binding site that specifically binds an IL-4 polypeptide, and a V_{H3} and V_{L3} domain pair that form a third antigen binding site that specifically binds a third target antigen. In some embodiments, the third target antigen is not an IL-4 polypeptide or an IL-13 polypeptide. In some embodiments, a trispecific binding protein of the present disclosure comprises a V_{H1} and V_{L1} domain pair that form a first antigen binding site that specifically binds an IL-13 polypeptide, a V_{H2} and V_{L2} domain pair that form a second antigen binding site that specifically binds an IL-4 polypeptide, and a V_{H3} and V_{L3} domain pair that form a third antigen binding site that specifically binds an OX40L polypeptide.

In some embodiments, a binding protein of the present disclosure comprises a V_{H1} and V_{L1} domain pair that form a first antigen binding site that specifically binds an IL-17 polypeptide. In some embodiments, a binding protein of the present disclosure comprises a V_{H2} and V_{L2} domain pair that form a second antigen binding site that specifically binds an IL-17 polypeptide. In some embodiments, a binding protein of the present disclosure comprises a V_{H3} and V_{L3} domain pair that form a third antigen binding site that specifically binds an IL-17 polypeptide. In some embodiments, a binding protein of the present disclosure comprises a V_{H1} and V_{L1} domain pair that form a first antigen binding site that specifically binds an IL-4 polypeptide, a V_{H2} and V_{L2} domain pair that form a second antigen binding site that specifically binds an IL-13 polypeptide, and a V_{H3} and V_{L3} domain pair that form a third antigen binding site that specifically binds an IL-17 polypeptide. In some embodiments, a binding protein of the present disclosure comprises a V_{H1} and V_{L1} domain pair that form a first antigen binding site that specifically binds an IL-13 polypeptide, a V_{H2} and V_{L2} domain pair that form a second antigen binding site that specifically binds an IL-4 polypeptide, and a V_{H3} and V_{L3} domain pair that form a third antigen binding site that specifically binds an IL-17 polypeptide.

In some embodiments, a trispecific binding protein of the present disclosure comprises a V_{H1} and V_{L1} domain pair that form a first antigen binding site that specifically binds aNKG2D polypeptide. In some embodiments, atrispecific binding protein of the present disclosure comprises a V_{H2} and V_{L2} domain pair that form a second antigen binding site that specifically binds a NKG2D polypeptide. In some embodiments, a trispecific binding protein of the present disclosure comprises a V_{H3} and V_{L3} domain pair that form a third antigen binding site that specifically binds a NKG2D polypeptide. In some embodiments, the antigen is expressed on the surface of a natural killer (NK) cell, e.g., a natural killer group 2D (NKG2D) polypeptide. In some embodiments, a trispecific binding protein of the present disclosure comprises a V_{H1} and V_{L1} domain pair that form a first antigen binding site that specifically binds an NKG2D polypeptide. In some embodiments, a trispecific binding protein of the present disclosure comprises a V_{H2} and V_{L2} domain pair that form a second antigen binding site that specifically binds anNKG2D polypeptide. In some embodiments, a trispecific binding protein of the present disclosure comprises a V_{H3} and V_{L3} domain pair that form a third antigen binding site that specifically binds an NKG2D polypeptide. In some embodiments, a trispecific binding protein of the present disclosure comprises a V_{H1} and V_{L1} domain pair that form a first antigen binding site that specifically binds an IL-4 polypeptide, a V_{H2} and V_{L2} domain pair that form a second antigen binding site that specifically binds an IL-13 polypeptide, and a V_{H3} and V_{L3} domain pair that form a third antigen binding site that specifically binds a TNFRSF polypeptide. In some embodiments, a trispecific binding protein of the present disclosure comprises a V_{H1} and V_{L1} domain pair that form a first antigen binding site that specifically binds an IL-4 polypeptide, a V_{H2} and V_{L2} domain pair that form a second antigen binding site that specifically binds an IL-13 polypeptide, and a V_{H3} and V_{L3} domain pair that form a third antigen binding site that specifically binds an immunoglobulin-like domain containing receptor superfamily polypeptide (e.g., CD47, KLRG1, or ICOS).

In some embodiments, a trispecific binding protein of the present disclosure is capable of inhibiting the function of one or more target proteins. In some embodiments, a trispecific binding protein of the present disclosure is capable of inhibiting the function of an IL-4 polypeptide and/or an IL-13 polypeptide. In some embodiments, a trispecific binding protein of the present disclosure is capable of inhibiting the function of an IL-4 polypeptide and an IL-13 polypeptide. In some embodiments, a trispecific binding protein of the present disclosure is capable of inhibiting the function of an OX40L polypeptide.

In some embodiments, a binding protein of the present disclosure is capable of inhibiting the function of one or more target proteins. In some embodiments, a binding protein of the present disclosure is capable of inhibiting the function of an IL-17 polypeptide, e.g., inhibiting binding of IL-17 to an IL-17 receptor, and/or inhibiting IL-17 receptor signaling mediated by IL-17 binding. In some embodiments, a binding protein of the present disclosure is capable of inhibiting the function of an IL-4 polypeptide, an IL-13 polypeptide, and/or an IL-17 polypeptide. In some embodiments, a binding protein of the present disclosure is capable of inhibiting the function of an IL-4 polypeptide, an IL-13 polypeptide, and an IL-17 polypeptide.

In some embodiments, a trispecific binding protein of the present disclosure is capable of inhibiting the function of one or more target proteins. In some embodiments, a trispecific binding protein of the present disclosure is capable of inhibiting the function of an NKG2D polypeptide, e.g., binding to MHC class I-related protein A (MICA) and/or MHC class I-related protein B (MICB) and/or activating NKG2D signaling. In some embodiments, a trispecific binding protein of the present disclosure is capable of inhibiting the function of an IL-4 polypeptide, an IL-13 polypeptide, and/or an NKG2D polypeptide. In some embodiments, a trispecific binding protein of the present disclosure is capable of inhibiting the function of an IL-4 polypeptide, an IL-13 polypeptide, and an NKG2D polypeptide.

In some embodiments, a trispecific binding protein of the present disclosure comprises a V_{H1} and V_{L1} domain pair that form a first antigen binding site that specifically binds an IL-4 polypeptide, a V_{H2} and V_{L2} domain pair that form a second antigen binding site that specifically binds an IL-13 polypeptide, and a V_{H3} and V_{L3} domain pair that form a third antigen binding site that specifically binds an OX40L polypeptide. Any of the anti-IL-4 binding sites described below (e.g., in Table 1), anti-IL-13 binding sites described below (e.g., in Table 2), or anti-OX40L binding sites described below (e.g., in Table 3) may be used in a trispecific binding protein of the present disclosure.

In some embodiments, the trispecific binding protein of the present disclosure comprises a V_{H1} and V_{L1} domain pair that form a first antigen binding site, a V_{H2} and V_{L2} domain pair that form a second antigen binding site, and a V_{H3} and V_{L3} domain pair that form a third antigen binding site.

In some embodiments, V_{H1} comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3; V_{L1} comprises a CDR-L₁ comprising the amino acid sequence of SEQ ID NO:6, a CDR-L2 comprising the amino acid sequence of SEQ ID NO:8, and a CDR-L3 comprising the amino acid sequence of SEQ ID NO:10; V_{H2} comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59; V_{L2} comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62; V_{H3} comprises a CDR-H1 comprising the amino acid sequence of SEQ ID NO:78, a CDR-H2 comprising the amino acid sequence of SEQ ID NO:79, and a CDR-H3 comprising the amino acid sequence of SEQ ID NO:80; and V_{L3} comprises a CDR-L1 comprising the amino acid sequence of SEQ ID NO:81, a CDR-L2 comprising the amino acid sequence of SEQ ID NO:82, and a CDR-L3 comprising the amino acid sequence of SEQ ID NO:83. In some embodiments, V_{H1} comprises the amino acid sequence of SEQ ID NO:34, V_{L1} comprises the amino acid sequence of SEQ ID NO:35, V_{H2} comprises the amino acid sequence of SEQ ID NO:70, V_{L2} comprises the amino acid sequence of SEQ ID NO:71, V_{H3} comprises the amino acid sequence of SEQ ID NO:91, and V_{L3} comprises the amino acid sequence of SEQ ID NO:92.

In some embodiments, a binding protein of the present disclosure comprises four polypeptide chains that form three antigen binding sites, wherein the first polypeptide chain comprises the amino acid sequence of SEQ ID NO:98 or an amino acid sequence that is at least 95% identical to the amino acid sequence of SEQ ID NO:98; the second polypeptide chain comprises the amino acid sequence of SEQ ID NO:99 or an amino acid sequence that is at least 95% identical to the amino acid sequence of SEQ ID NO:99; the third polypeptide chain comprises the amino acid sequence of SEQ ID NO:100 or an amino acid sequence that is at least 95% identical to the amino acid sequence of SEQ ID NO: 100; and the fourth polypeptide chain comprises the amino acid sequence of SEQ ID NO:101 or an amino acid sequence that is at least 95% identical to the amino acid sequence of SEQ ID NO:101. In some embodiments, a binding protein of the present disclosure comprises four polypeptide chains that form three antigen binding sites, wherein the first polypeptide chain comprises the amino acid sequence of SEQ ID NO:98; the second polypeptide chain comprises the amino acid sequence of SEQ ID NO:99; the third polypeptide chain comprises the amino acid sequence of SEQ ID NO:100; and the fourth polypeptide chain comprises the amino acid sequence of SEQ ID NO:101.

Any of the anti-IL-4 binding sites described below (e.g., in Table 1), anti-IL-13 binding sites described below (e.g., in Table 2), or anti-IL-17 binding sites described below (e.g., in Table 48) may be used in a binding protein of the present disclosure.

In some embodiments, V_{H1} comprises a CDR-H1 comprising the amino acid sequence of SEQ ID NO:12, a CDR-H2 comprising the amino acid sequence of SEQ ID NO:13, and a CDR-H3 comprising the amino acid sequence of SEQ ID NO:14; V_{L1} comprises a CDR-L₁ comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO:117, a CDR-H2 comprising the amino acid sequence of SEQ ID NO:118, and a CDR-H3 comprising the amino acid sequence of SEQ ID NO:119; and V_{L3} comprises a CDR-L1 comprising the amino acid sequence of SEQ ID NO: 114, a CDR-L2 comprising the amino acid sequence of TFF, and a CDR-L3 comprising the amino acid sequence of SEQ ID NO:116. In some embodiments, V_{H1} comprises a CDR-H1 comprising the amino acid sequence of SEQ ID NO:12, a CDR-H2 comprising the amino acid sequence of SEQ ID NO: 13, and a CDR-H3 comprising the amino acid sequence of SEQ ID NO:14; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L₁ comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO:123, a CDR-H2 comprising the amino acid sequence of SEQ ID NO: 124, and a CDR-H3 comprising the amino acid sequence of SEQ ID NO:125; and V_{L3} comprises a CDR-L1 comprising the amino acid sequence of SEQ ID NO:120, a CDR-L2 comprising the amino acid sequence of GTY, and a CDR-L3 comprising the amino acid sequence of SEQ ID NO:122. In some embodiments, V_{H1} comprises a CDR-H1 comprising the amino acid sequence of SEQ ID NO:12, a CDR-H2 comprising the amino acid sequence of SEQ ID NO:13, and a CDR-H3 comprising the amino acid sequence of SEQ ID NO:14; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; and V_{L3} comprises a CDR-L1 comprising the amino acid sequence of SEQ ID NO:126, a CDR-L2 comprising the amino acid sequence of GTY, and a CDR-L3 comprising the amino acid sequence of SEQ ID NO:128. In some embodiments, V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:31; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO: 117, a CDR-H2 comprising the amino acid sequence of SEQ ID NO: 118, and a CDR-H3 comprising the amino acid sequence of SEQ ID NO:119; and V_{L3} comprises a CDR-L1 comprising the amino acid sequence of SEQ ID NO:114, a CDR-L2 comprising the amino acid sequence of TFF, and a CDR-L3 comprising the amino acid sequence of SEQ ID NO:116. In some embodiments, V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:31; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO:123, a CDR-H2 comprising the amino acid sequence of SEQ ID NO: 124, and a CDR-H3 comprising the amino acid sequence of SEQ ID NO:125; and V_{L3} comprises a CDR-L1 comprising the amino acid sequence of SEQ ID NO:120, a CDR-L2 comprising the amino acid sequence of GTY, and a CDR-L3 comprising the amino acid sequence of SEQ ID NO:122. In some embodiments, V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:31; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO: 129, a CDR-H2 comprising the amino acid sequence of SEQ ID NO: 130, and a CDR-H3 comprising the amino acid sequence of SEQ ID NO:131; and V_{L3} comprises a CDR-L1 comprising the amino acid sequence of SEQ ID NO:126, a CDR-L2 comprising the amino acid sequence of GTY, and a CDR-L3 comprising the amino acid sequence of SEQ ID NO:128.

In some embodiments, V_{H1} comprises an amino acid sequence that is at least 95%, at least 96%, at least 97%, at least 98%, at least 99%, or 100% identical to the amino acid sequence of SEQ ID NO:40; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:132; and V_{L3} comprises an amino acid sequence that is at least 95%, at least 96%, at least 97%, at least 98%, at least 99%, or 100% identical to the amino acid sequence of SEQ ID NO:133. In some embodiments, V_{H1} comprises an amino acid sequence that is at least 95%, at least 96%, at least 97%, at least 98%, at least 99%, or 100% identical to the amino acid sequence of SEQ ID NO:40; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:134; and V_{L3} comprises an amino acid sequence that is at least 95%, at least 96%, at least 97%, at least 98%, at least 99%, or 100% identical to the amino acid sequence of SEQ ID NO:135. In some embodiments, V_{H1} comprises an amino acid sequence that is at least 95%, at least 96%, at least 97%, at least 98%, at least 99%, or 100% identical to the amino acid sequence of SEQ ID NO:40; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:136; and V_{L3} comprises an amino acid sequence that is at least 95%, at least 96%, at least 97%, at least 98%, at least 99%, or 100% identical to the amino acid sequence of SEQ ID NO:137. In some embodiments, V_{H1} comprises an amino acid sequence that is at least 95%, at least 96%, at least 97%, at least 98%, at least 99%, or 100% identical to the amino acid sequence of SEQ ID NO:53; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:132; and V_{L3} comprises an amino acid sequence that is at least 95%, at least 96%, at least 97%, at least 98%, at least 99%, or 100% identical to the amino acid sequence of SEQ ID NO:133. In some embodiments, V_{H1} comprises an amino acid sequence that is at least 95%, at least 96%, at least 97%, at least 98%, at least 99%, or 100% identical to the amino acid sequence of SEQ ID NO:53; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:134; and V_{L3} comprises an amino acid sequence that is at least 95%, at least 96%, at least 97%, at least 98%, at least 99%, or 100% identical to the amino acid sequence of SEQ ID NO:135. In some embodiments, V_{H1} comprises an amino acid sequence that is at least 95%, at least 96%, at least 97%, at least 98%, at least 99%, or 100% identical to the amino acid sequence of SEQ ID NO:53; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:136; and V_{L3} comprises an amino acid sequence that is at least 95%, at least 96%, at least 97%, at least 98%, at least 99%, or 100% identical to the amino acid sequence of SEQ ID NO:137.

In some embodiments, V_{H1} contains the same CDR-H1 sequence, the same CDR-H2 sequence, and the same CDR-H3 sequence, as the amino acid sequence of SEQ ID NO:40; V_{L1} contains the same CDR-L1 sequence, the same CDR-L2 sequence, and the same CDR-L3 sequence, as the amino acid sequence of SEQ ID NO:41; V_{H2} contains the same CDR-H1 sequence, the same CDR-H2 sequence, and the same CDR-H3 sequence, as the amino acid sequence of SEQ ID NO:72; V_{L2} contains the same CDR-L1 sequence, the same CDR-L2 sequence, and the same CDR-L3 sequence, as the amino acid sequence of SEQ ID NO:73; V_{H3} contains the same CDR-H1 sequence, the same CDR-H2 sequence, and the same CDR-H3 sequence, as the amino acid sequence of SEQ ID NO:132; and V_{L3} contains the same CDR-L1 sequence, the same CDR-L2 sequence, and the same CDR-L3 sequence, as the amino acid sequence of SEQ ID NO:133. In some embodiments, V_{H1} contains the same CDR-H1 sequence, the same CDR-H2 sequence, and the same CDR-H3 sequence, as the amino acid sequence of SEQ ID NO:40; V_{L1} contains the same CDR-L1 sequence, the same CDR-L2 sequence, and the same CDR-L3 sequence, as the amino acid sequence of SEQ ID NO:41; V_{H2} contains the same CDR-H1 sequence, the same CDR-H2 sequence, and the same CDR-H3 sequence, as the amino acid sequence of SEQ ID NO:72; V_{L2} contains the same CDR-L1 sequence, the same CDR-L2 sequence, and the same CDR-L3 sequence, as the amino acid sequence of SEQ ID NO:73; V_{H3} contains the same CDR-H1 sequence, the same CDR-H2 sequence, and the same CDR-H3 sequence, as the amino acid sequence of SEQ ID NO:134; and V_{L3} contains the same CDR-L1 sequence, the same CDR-L2 sequence, and the same CDR-L₃ sequence, as the amino acid sequence of SEQ ID NO:135. In some embodiments, V_{H1} contains the same CDR-H1 sequence, the same CDR-H2 sequence, and the same CDR-H3 sequence, as the amino acid sequence of SEQ ID NO:40; V_{L1} contains the same CDR-L1 sequence, the same CDR-L2 sequence, and the same CDR-L3 sequence, as the amino acid sequence of SEQ ID NO:41; V_{H2} contains the same CDR-H1 sequence, the same CDR-H2 sequence, and the same CDR-H3 sequence, as the amino acid sequence of SEQ ID NO:72; V_{L2} contains the same CDR-L1 sequence, the same CDR-L2 sequence, and the same CDR-L3 sequence, as the amino acid sequence of SEQ ID NO:73; V_{H3} contains the same CDR-H1 sequence, the same CDR-H2 sequence, and the same CDR-H3 sequence, as the amino acid sequence of SEQ ID NO: 136; and V_{L3} contains the same CDR-L1 sequence, the same CDR-L₂ sequence, and the same CDR-L3 sequence, as the amino acid sequence of SEQ ID NO:137. In some embodiments, V_{H1} contains the same CDR-H1 sequence, the same CDR-H2 sequence, and the same CDR-H3 sequence, as the amino acid sequence of SEQ ID NO:53; V_{L1} contains the same CDR-L1 sequence, the same CDR-L2 sequence, and the same CDR-L3 sequence, as the amino acid sequence of SEQ ID NO:41; V_{H2} contains the same CDR-H1 sequence, the same CDR-H2 sequence, and the same CDR-H3 sequence, as the amino acid sequence of SEQ ID NO:72; V_{L2} contains the same CDR-L1 sequence, the same CDR-L2 sequence, and the same CDR-L3 sequence, as the amino acid sequence of SEQ ID NO:73; V_{H3} contains the same CDR-H1 sequence, the same CDR-H2 sequence, and the same CDR-H3 sequence, as the amino acid sequence of SEQ ID NO:132; and V_{L3} contains the same CDR-L1 sequence, the same CDR-L2 sequence, and the same CDR-L3 sequence, as the amino acid sequence of SEQ ID NO: 133. In some embodiments, V_{H1} contains the same CDR-H1 sequence, the same CDR-H2 sequence, and the same CDR-H3 sequence, as the amino acid sequence of SEQ ID NO:53; V_{L1} contains the same CDR-L1 sequence, the same CDR-L2 sequence, and the same CDR-L3 sequence, as the amino acid sequence of SEQ ID NO:41; V_{H2} contains the same CDR-H1 sequence, the same CDR-H2 sequence, and the same CDR-H3 sequence, as the amino acid sequence of SEQ ID NO:72; V_{L2} contains the same CDR-L1 sequence, the same CDR-L2 sequence, and the same CDR-L3 sequence, as the amino acid sequence of SEQ ID NO:73; V_{H3} contains the same CDR-H1 sequence, the same CDR-H2 sequence, and the same CDR-H3 sequence, as the amino acid sequence of SEQ ID NO:134; and V_{L3} contains the same CDR-L1 sequence, the same CDR-L2 sequence, and the same CDR-L3 sequence, as the amino acid sequence of SEQ ID NO:135. In some embodiments, V_{H1} contains the same CDR-H1 sequence, the same CDR-H2 sequence, and the same CDR-H3 sequence, as the amino acid sequence of SEQ ID NO:53; V_{L1} contains the same CDR-L1 sequence, the same CDR-L2 sequence, and the same CDR-L3 sequence, as the amino acid sequence of SEQ ID NO:41; V_{H2} contains the same CDR-H1 sequence, the same CDR-H2 sequence, and the same CDR-H3 sequence, as the amino acid sequence of SEQ ID NO:72; V_{L2} contains the same CDR-L1 sequence, the same CDR-L2 sequence, and the same CDR-L3 sequence, as the amino acid sequence of SEQ ID NO:73; V_{H3} contains the same CDR-H1 sequence, the same CDR-H2 sequence, and the same CDR-H3 sequence, as the amino acid sequence of SEQ ID NO:136; and V_{L3} contains the same CDR-L1 sequence, the same CDR-L2 sequence, and the same CDR-L₃ sequence, as the amino acid sequence of SEQ ID NO:137.

In some embodiments, a binding protein of the present disclosure comprises four polypeptide chains that form three antigen binding sites, wherein the first polypeptide chain comprises the amino acid sequence of SEQ ID NO:138 or an amino acid sequence that is at least 95%, at least 96%, at least 97%, at least 98%, or at least 99% identical to the amino acid sequence of SEQ ID NO: 138; the second polypeptide chain comprises the amino acid sequence of SEQ ID NO:139 or an amino acid sequence that is at least 95%, at least 96%, at least 97%, at least 98%, or at least 99% identical to the amino acid sequence of SEQ ID NO:139; the third polypeptide chain comprises the amino acid sequence of SEQ ID NO:140 or an amino acid sequence that is at least 95%, at least 96%, at least 97%, at least 98%, or at least 99% identical to the amino acid sequence of SEQ ID NO: 140; and the fourth polypeptide chain comprises the amino acid sequence of SEQ ID NO:141 or an amino acid sequence that is at least 95%, at least 96%, at least 97%, at least 98%, or at least 99% identical to the amino acid sequence of SEQ ID NO:141. In some embodiments, a binding protein of the present disclosure comprises four polypeptide chains that form three antigen binding sites, wherein the first polypeptide chain comprises the amino acid sequence of SEQ ID NO:138; the second polypeptide chain comprises the amino acid sequence of SEQ ID NO: 139; the third polypeptide chain comprises the amino acid sequence of SEQ ID NO:140; and the fourth polypeptide chain comprises the amino acid sequence of SEQ ID NO: 141.

In some embodiments, a binding protein of the present disclosure comprises four polypeptide chains that form three antigen binding sites, wherein the first polypeptide chain comprises the amino acid sequence of SEQ ID NO:142 or an amino acid sequence that is at least 95%, at least 96%, at least 97%, at least 98%, or at least 99% identical to the amino acid sequence of SEQ ID NO: 142; the second polypeptide chain comprises the amino acid sequence of SEQ ID NO:143 or an amino acid sequence that is at least 95%, at least 96%, at least 97%, at least 98%, or at least 99% identical to the amino acid sequence of SEQ ID NO:143; the third polypeptide chain comprises the amino acid sequence of SEQ ID NO:144 or an amino acid sequence that is at least 95%, at least 96%, at least 97%, at least 98%, or at least 99% identical to the amino acid sequence of SEQ ID NO: 144; and the fourth polypeptide chain comprises the amino acid sequence of SEQ ID NO:145 or an amino acid sequence that is at least 95%, at least 96%, at least 97%, at least 98%, or at least 99% identical to the amino acid sequence of SEQ ID NO:145. In some embodiments, a binding protein of the present disclosure comprises four polypeptide chains that form three antigen binding sites, wherein the first polypeptide chain comprises the amino acid sequence of SEQ ID NO:142; the second polypeptide chain comprises the amino acid sequence of SEQ ID NO: 143; the third polypeptide chain comprises the amino acid sequence of SEQ ID NO:144; and the fourth polypeptide chain comprises the amino acid sequence of SEQ ID NO:145.

In some embodiments, a binding protein of the present disclosure comprises four polypeptide chains that form three antigen binding sites, wherein the first polypeptide chain comprises the amino acid sequence of SEQ ID NO:146 or an amino acid sequence that is at least 95%, at least 96%, at least 97%, at least 98%, or at least 99% identical to the amino acid sequence of SEQ ID NO: 146; the second polypeptide chain comprises the amino acid sequence of SEQ ID NO:147 or an amino acid sequence that is at least 95%, at least 96%, at least 97%, at least 98%, or at least 99% identical to the amino acid sequence of SEQ ID NO:147; the third polypeptide chain comprises the amino acid sequence of SEQ ID NO:148 or an amino acid sequence that is at least 95%, at least 96%, at least 97%, at least 98%, or at least 99% identical to the amino acid sequence of SEQ ID NO: 148; and the fourth polypeptide chain comprises the amino acid sequence of SEQ ID NO:149 or an amino acid sequence that is at least 95%, at least 96%, at least 97%, at least 98%, or at least 99% identical to the amino acid sequence of SEQ ID NO:149. In some embodiments, a binding protein of the present disclosure comprises four polypeptide chains that form three antigen binding sites, wherein the first polypeptide chain comprises the amino acid sequence of SEQ ID NO:146; the second polypeptide chain comprises the amino acid sequence of SEQ ID NO: 147; the third polypeptide chain comprises the amino acid sequence of SEQ ID NO:148; and the fourth polypeptide chain comprises the amino acid sequence of SEQ ID NO: 149.

In some embodiments, a binding protein of the present disclosure comprises four polypeptide chains that form three antigen binding sites, wherein the first polypeptide chain comprises the amino acid sequence of SEQ ID NO:150 or an amino acid sequence that is at least 95%, at least 96%, at least 97%, at least 98%, or at least 99% identical to the amino acid sequence of SEQ ID NO: 150; the second polypeptide chain comprises the amino acid sequence of SEQ ID NO:151 or an amino acid sequence that is at least 95%, at least 96%, at least 97%, at least 98%, or at least 99% identical to the amino acid sequence of SEQ ID NO:151; the third polypeptide chain comprises the amino acid sequence of SEQ ID NO:152 or an amino acid sequence that is at least 95%, at least 96%, at least 97%, at least 98%, or at least 99% identical to the amino acid sequence of SEQ ID NO: 152; and the fourth polypeptide chain comprises the amino acid sequence of SEQ ID NO:153 or an amino acid sequence that is at least 95%, at least 96%, at least 97%, at least 98%, or at least 99% identical to the amino acid sequence of SEQ ID NO:153. In some embodiments, a binding protein of the present disclosure comprises four polypeptide chains that form three antigen binding sites, wherein the first polypeptide chain comprises the amino acid sequence of SEQ ID NO:150; the second polypeptide chain comprises the amino acid sequence of SEQ ID NO:151; the third polypeptide chain comprises the amino acid sequence of SEQ ID NO:152; and the fourth polypeptide chain comprises the amino acid sequence of SEQ ID NO: 153.

Any of the anti-IL-4 binding sites described below (e.g., in Table 1), anti-IL-13 binding sites described below (e.g., in Table 2), or anti-NKG2D binding sites described below (e.g., in Table 49) may be used in a trispecific binding protein of the present disclosure.

In some embodiments, V_{H1} comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, V_{L1} comprises a CDR-L₁ comprising the amino acid sequence of SEQ ID NO:7, a CDR-L2 comprising the amino acid sequence of SEQ ID NO:9, and a CDR-L3 comprising the amino acid sequence of SEQ ID NO:11, V_{H2} comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, V_{L2} comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62, V_{H3} comprises a CDR-H1 comprising the amino acid sequence of SEQ ID NO:199, a CDR-H2 comprising the amino acid sequence of SEQ ID NO:200, and a CDR-H3 comprising the amino acid sequence of SEQ ID NO:201, and V_{L3} comprises a CDR-L1 comprising the amino acid sequence of SEQ ID NO:202, a CDR-L2 comprising the amino acid sequence of SEQ ID NO:203, and a CDR-L3 comprising the amino acid sequence of SEQ ID NO:204. In some embodiments, V_{H1} comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3, V_{L1} comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11, V_{H2} comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, V_{L2} comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62, V_{H3} comprises a CDR-H1 comprising the amino acid sequence of SEQ ID NO:193, a CDR-H2 comprising the amino acid sequence of SEQ ID NO:194, and a CDR-H3 comprising the amino acid sequence of SEQ ID NO:195, and V_{L3} comprises a CDR-L1 comprising the amino acid sequence of SEQ ID NO:196, a CDR-L₂ comprising the amino acid sequence of ATS, and a CDR-L3 comprising the amino acid sequence of SEQ ID NO:198. In some embodiments, V_{H1} comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, V_{L1} comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11, V_{H2} comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, V_{L2} comprises a CDR-L₁ comprising the amino acid sequence of SEQ ID NO:60, a CDR-L2 comprising the amino acid sequence of SEQ ID NO:61, and a CDR-L3 comprising the amino acid sequence of SEQ ID NO:62, V_{H3} comprises a CDR-H1 comprising the amino acid sequence of SEQ ID NO: 193, a CDR-H2 comprising the amino acid sequence of SEQ ID NO:194, and a CDR-H3 comprising the amino acid sequence of SEQ ID NO:195, and V_{L3} comprises a CDR-L1 comprising the amino acid sequence of SEQ ID NO:196, a CDR-L2 comprising the amino acid sequence of ATS, and a CDR-L3 comprising the amino acid sequence of SEQ ID NO: 198.

In some embodiments, V_{H1} comprises an amino acid sequence that is at least 95%, at least 96%, at least 97%, at least 98%, at least 99%, or 100% identical to the amino acid sequence of SEQ ID NO:53, V_{L1} comprises an amino acid sequence that is at least 95%, at least 96%, at least 97%, at least 98%, at least 99%, or 100% identical to the amino acid sequence of SEQ ID NO:41, V_{H2} comprises an amino acid sequence that is at least 95%, at least 96%, at least 97%, at least 98%, at least 99%, or 100% identical to the amino acid sequence of SEQ ID NO:72, V_{L2} comprises an amino acid sequence that is at least 95%, at least 96%, at least 97%, at least 98%, at least 99%, or 100% identical to the amino acid sequence of SEQ ID NO:73, V_{H3} comprises an amino acid sequence that is at least 95%, at least 96%, at least 97%, at least 98%, at least 99%, or 100% identical to the amino acid sequence of SEQ ID NO:240, and V_{L3} comprises an amino acid sequence that is at least 95%, at least 96%, at least 97%, at least 98%, at least 99%, or 100% identical to the amino acid sequence of SEQ ID NO:241. In some embodiments, V_{H1} comprises an amino acid sequence that is at least 95%, at least 96%, at least 97%, at least 98%, at least 99%, or 100% identical to the amino acid sequence of SEQ ID NO:40, V_{L1} comprises an amino acid sequence that is at least 95%, at least 96%, at least 97%, at least 98%, at least 99%, or 100% identical to the amino acid sequence of SEQ ID NO:41, V_{H2} comprises an amino acid sequence that is at least 95%, at least 96%, at least 97%, at least 98%, at least 99%, or 100% identical to the amino acid sequence of SEQ ID NO:72, V_{L2} comprises an amino acid sequence that is at least 95%, at least 96%, at least 97%, at least 98%, at least 99%, or 100% identical to the amino acid sequence of SEQ ID NO:73, V_{H3} comprises an amino acid sequence that is at least 95%, at least 96%, at least 97%, at least 98%, at least 99%, or 100% identical to the amino acid sequence of SEQ ID NO:237, and V_{L3} comprises an amino acid sequence that is at least 95%, at least 96%, at least 97%, at least 98%, at least 99%, or 100% identical to the amino acid sequence of SEQ ID NO:238. In some embodiments, V_{H1} comprises an amino acid sequence that is at least 95%, at least 96%, at least 97%, at least 98%, at least 99%, or 100% identical to the amino acid sequence of SEQ ID NO:53, V_{L1} comprises an amino acid sequence that is at least 95%, at least 96%, at least 97%, at least 98%, at least 99%, or 100% identical to the amino acid sequence of SEQ ID NO:41, V_{H2} comprises an amino acid sequence that is at least 95%, at least 96%, at least 97%, at least 98%, at least 99%, or 100% identical to the amino acid sequence of SEQ ID NO:72, V_{L2} comprises an amino acid sequence that is at least 95%, at least 96%, at least 97%, at least 98%, at least 99%, or 100% identical to the amino acid sequence of SEQ ID NO:73, V_{H3} comprises an amino acid sequence that is at least 95%, at least 96%, at least 97%, at least 98%, at least 99%, or 100% identical to the amino acid sequence of SEQ ID NO:239, and V_{L3} comprises an amino acid sequence that is at least 95%, at least 96%, at least 97%, at least 98%, at least 99%, or 100% identical to the amino acid sequence of SEQ ID NO:238.

In some embodiments, a binding protein of the present disclosure comprises four polypeptide chains that form three antigen binding sites, wherein the first polypeptide chain of the binding protein comprises an amino acid sequence that is at least 95%, at least 96%, at least 97%, at least 98%, at least 99%, or 100% identical to the amino acid sequence of SEQ ID NO:242, the second polypeptide chain of the binding protein comprises an amino acid sequence that is at least 95%, at least 96%, at least 97%, at least 98%, at least 99%, or 100% identical to the amino acid sequence of SEQ ID NO:243, the third polypeptide chain of the binding protein comprises an amino acid sequence that is at least 95%, at least 96%, at least 97%, at least 98%, at least 99%, or 100% identical to the amino acid sequence of SEQ ID NO:244, and the fourth polypeptide chain of the binding protein comprises an amino acid sequence that is at least 95%, at least 96%, at least 97%, at least 98%, at least 99%, or 100% identical to the amino acid sequence of SEQ ID NO:245. In some embodiments, a binding protein of the present disclosure comprises four polypeptide chains that form three antigen binding sites, wherein the first polypeptide chain of the binding protein comprises an amino acid sequence that is at least 95%, at least 96%, at least 97%, at least 98%, at least 99%, or 100% identical to the amino acid sequence of SEQ ID NO:246, the second polypeptide chain of the binding protein comprises an amino acid sequence that is at least 95%, at least 96%, at least 97%, at least 98%, at least 99%, or 100% identical to the amino acid sequence of SEQ ID NO:247, the third polypeptide chain of the binding protein comprises an amino acid sequence that is at least 95%, at least 96%, at least 97%, at least 98%, at least 99%, or 100% identical to the amino acid sequence of SEQ ID NO:248, and the fourth polypeptide chain of the binding protein comprises an amino acid sequence that is at least 95%, at least 96%, at least 97%, at least 98%, at least 99%, or 100% identical to the amino acid sequence of SEQ ID NO:249. In some embodiments, a binding protein of the present disclosure comprises four polypeptide chains that form three antigen binding sites, wherein the first polypeptide chain of the binding protein comprises an amino acid sequence that is at least 95%, at least 96%, at least 97%, at least 98%, at least 99%, or 100% identical to the amino acid sequence of SEQ ID NO:250, the second polypeptide chain of the binding protein comprises an amino acid sequence that is at least 95%, at least 96%, at least 97%, at least 98%, at least 99%, or 100% identical to the amino acid sequence of SEQ ID NO:251, the third polypeptide chain of the binding protein comprises an amino acid sequence that is at least 95%, at least 96%, at least 97%, at least 98%, at least 99%, or 100% identical to the amino acid sequence of SEQ ID NO:252, and the fourth polypeptide chain of the binding protein comprises an amino acid sequence that is at least 95%, at least 96%, at least 97%, at least 98%, at least 99%, or 100% identical to the amino acid sequence of SEQ ID NO:253.

Table 4 indicates the sequences of four chains of an exemplary trispecific binding protein. In some embodiments, a binding protein of the present disclosure comprises one, two, three, or all four polypeptide chains of a single binding protein described in Table 4. In some embodiments, a binding protein of the present disclosure comprises one, two, three, or four polypeptide chains that are at least 95%, at least 96%, at least 97%, at least 98%, or at least 99% identical to one, two, three, or all four polypeptide chains of a single binding protein described in Table 4.

TABLE 4
Exemplary trispecific binding protein sequences.

| | | SEQ |
|---|---|---|
| | | ID |
| Description | Sequence | NO |
| 1^{st} chain (CODV | SYELTQPPSVSVSPGQTARITCSGDALPKQYAYWYQKKPGQAPVLVIYKDS | 98 |
| LC) | ERPSGIPERFSGSSKGTTVTLTISGVQAEDEADYYCQSADSSGTYWVFGGG | |
| | TKLTVLGGGGSGGGGSDIQMTQSPSSLSASVGDRVTITCQASQSISNYLN | |
| | WYQKKPGKAPKLLIYDASNSETGVPSRFSGSGSGTDFTFTISSLQPEDIATY | |
| | YCQQYYDLYKFGGGTKVEIKGGGGSGGGGSRTVAAPAVFIFPPSKKQLKS | |
| | GTASVVCLLKNFYPREAKVQWKVDNALQSGNSQESVTEQDSKDSTYSLSS | |
| | TLTLSKADYEKHKVYACEVTHQGLSSPVTKSFNRGEC | |
| 2^{nd} chain (CODV | QLQLQESGPGLKKPSETLSLTCTVSGGSISSIPYYWGWIREPPGKGLEWIGS | 99 |
| HC) | IYPSGSTDYNPSLKSRVTISVDTSKNQFSLKLSSVTAADTAVYYCARGGARD | |
| | RIDHAFSIWGQGTMVTVSSEVQLLESGGGLVQPGGSLRLSCAASGFTFSSY | |
| | AMSWVREAPGKGLEWVSAISGSGGSTYYADSVKGRFTISRDNSKNTLYLQ | |
| | MNSLRAEDTAVYYCAREYYYDPLQYYHYYYGMDVWGQGTTVTVSSASTK | |
| | GPSVFPLAPSSKSTSGGTAALGCLVKDYFPEPVTVSWNSGALTSGVHTFPA | |
| | VLQSSGLYSLSSVVEVPSSSLGTQTYICNVNHKPSNTKVDEKVEPDSCDKTH | |
| | TCPPCPAPEAAGGPSVFLFPPKPKDTLMISRDPEVTCVVVDVSHEDPEVKF | |
| | NWYVDGVEVHNAKTKPREEQYNSTYRVVSVLQVLHQDWLNGKEYKCKV | |
| | SNKALPAPIEKTISKAKGQPREPQVYTLPPCRDELTKNQVSLWCLVKGFYPS | |
| | DIAVEWESNGQPENNYKTTPPVLDSDGSFFLYSKLTVDKSRWQQGNVFS | |
| | CSVMHEALHNHYTQKSLSLSPG | |
| 3^{rd} chain (Fab | EVQLVESGGGFVQPGMSLRLSCAASGFTFSSYAMTWVRKAPGKGLEWV | 100 |
| HC) | SDISGSGRSIFYADSVKGRFTISRDNSKRTLYLQMNSLRAEDTAIYYCAKDL | |
| | GFCSGASCNEADYWGQGTLVTVSSASTKGPSVFPLAPSSKSTSGGTAALG | |
| | CLVKDYFPEPVTVSWNSGALTSGVHTFPAVLQSSGLYSLSSVVTVPSSSLGT | |
| | QTYICNVNHKPSNTKVDKKVEPKSCDKTHTCPPCPAPEAAGGPSVFLFPPK | |
| | PKDTLMISRDPEVTCVVVDVSHEDPEVKFNWYVDGVEVHNAKTKPREEQ | |
| | YNSTYRVVSVLQVLHQDWLNGKEYKCKVSNKALPAPIEKTISKAKGQPREP | |
| | QVCTLPPSRDELTKNQVSLSCAVKGFYPSDIAVEWESNGQPENNYKTTPP | |
| | VLDSDGSFFLVSKLTVDKSRWQQGNVFSCSVMHEALHNRFTQKSLSLSPG | |
| 4^{th} chain (Fab LC) | DIQMTQSPSSLSASVGDRVTITCRASQGIRNDLDWYQEKPGKAPKRLIYAT | 101 |
| | SGLQSGVPSRFSGSGSGTEFTLTISSLQPEDFATYYCLQHSRYPWTFGQGT | |
| | KVEIKRTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQGLSSP | |
| | VTKSFNRGEC | |
| (A-IL4_VR- | SYELTQPPSVSVSPGQTARITCSGDALPKQYAYWYQEKPGQAPVLVIYK | 138 |
| 62824 X A- | DSERPSGIPERFSGSSKGTTVTLTISGVQAEDEADYYCQSADSSGTYWV | |
| IL13_VR- | FGGGTKLTVLGGGGSGGGGSDIQMTQSPSSLSASVGDRVTITCQASQDI | |
| 62822)-CM1- | SNYLNWYQEKPGKAPKLLIYDASNLETGVPSRFSGSGSGTDFTFTISSLQ | |
| CODV X A- | PEDIATYYCQQYYDLYTFGGGTKVEIKGGGGSGGGGSRTVAAPSVFIFP | |
| IL17A_VR- | PSDEQLKSGTASVVCLLNNFYPREAKVQWKVDNALQSGNSQESVTEQD | |
| 92018-CM2)- | SKDSTYSLSSTLTLSKADYEKHKVYACEVTHQGLSSPVTKSFNRGEC | |
| huIgG1- | | |
| CR3_NN3_LAL | | |
| A_DQ_dk | | |
| Chain 1 | | |
| (A-IL4 VR- | QLQLQESGPGLVKPSETLSLTCTVSGGSISSESYYWGWIRKPPGKGLEW | 139 |
| 62824 X A- | IGSIYYSGSTDYNPSLKSRVTISVDTSKNQFSLKLSSVTAADTAVYYCARG | |
| IL13_VR- | GASDRIDHAFEIWGQGTMVTVSSEVQLLESGGGLVQPGGSLRLSCAASG | |
| 62822)-CM1- | FTFSSYAMSWVRKAPGKGLEWVSAISGSGGSTYYADSVKGRFTISRDNS | |
| CODV X A- | KNTLYLQMNSLRAEDTAVYYCAREYYYDPLQYYHYYYGMDVWGQGTTV | |
| IL17A_VR- | TVSSASTKGPSVFPLAPSSKSTSGGTAALGCLVKDYFPEPVTVSWNSGA | |
| 92018-CM2)- | LTSGVHTFPAVLQSSGLYSLSSVVTVPSSSLGTQTYICNVNHKPSNTKVD | |
| huIgG1- | KKVEPKSCDKTHTCPPCPAPEAAGGPSVFLFPPKPKDTLMISRDPEVTCV | |
| CR3_NN3_LAL | VVDVSHEDPEVKFNWYVDGVEVHNAKTKPREEQYNSTYRVVSVLQVLH | |
| A_DQ_dk | QDWLNGKEYKCKVSNKALPAPIEKTISKAKGQPREPQVYTLPPCRDELTK | |
| Chain 2 | NQVSLWCLVKGFYPSDIAVEWESNGQPENNYKTTPPVLDSDGSFFLYSK | |
| | LTVDKSRWQQGNVFSCSVMHEALHNHYTQKSLSLSPG | |
| (A-IL4 VR- | | |
| 62824 X A- | QVQLQQWGAGLLKPSETLSLTCAVFGGSFDGYYWNWIREPPGKGLEWIG | 140 |
| IL13_VR- | EIFHIGGTIYNPSLKSRVTISEDTSKNQFSLRLNSVTAADTATYYCARGI | |
| 62822)-CM1- | TMGRGNYYYYGLDVWGQGTPVTVSSASTKGPSVFPLAPSSKSTSGGTAA | |
| CODV X A- | LGCLVKDYFPEPVTVSWNSGALTSGVHTFPAVLQSSGLYSLSSVVEVPS | |
| IL17A_VR- | SSLGTQTYICNVNHKPSNTKVDEKVEPDSCDKTHTCPPCPAPEAAGGPS | |
| 92018-CM2)- | VFLFPPKPKDTLMISRDPEVTCVVVDVSHEDPEVKFNWYVDGVEVHNAK | |
| huIgG1- | TKPREEQYNSTYRVVSVLQVLHQDWLNGKEYKCKVSNKALPAPIEKTISK | |
| CR3_NN3_LAL | AKGQPREPQVCTLPPSRDELTKNQVSLSCAVKGFYPSDIAVEWESNGQP | |
| A_DQ_dk | ENNYKTTPPVLDSDGSFFLVSKLTVDKSRWQQGNVFSCSVMHEALHNR | |
| Chain 3 | FTQKSLSLSPG | |
| (A-IL4_VR- | DIQMTQSPSSLSASVGDRVTITCRASQDIKNDLGWYQKKPGKAPKFLIFG | 141 |
| 62824 X A- | TYSLQSGVPSRFSGSGSGTEFTLTISSLQPEDFATYYCLQDNNYPRTFG | |
| IL13_VR- | QGTKVEIKRTVAAPAVFIFPPSKKQLKSGTASVVCLLKNFYPREAKVQWK | |
| 62822)-CM1- | VDNALQSGNSQESVTEQDSKDSTYSLSSTLTLSKADYEKHKVYACEVTH | |
| CODV X A- | QGLSSPVTKSFNRGEC | |
| IL17A_VR- | | |
| 92018-CM2)- | | |
| huIgG1- | | |
| CR3_NN3_LAL | | |
| A_DQ_dk | | |
| Chain 4 | | |
| (a-IL4_VR- | SYELTQPPSVSVSPGQTARITCSGDALPKQYAYWYQEKPGQAPVLVIYK | 142 |
| 93303 X a- | DSERPSGIPERFSGSSKGTTVTLTISGVQAEDEADYYCQSADSSGTYWV | |
| IL13_VR- | FGGGTKLTVLGGGGGGGGSDIQMTQSPSSLSASVGDRVTITCQASQDI | |
| 62822)-CM1- | SNYLNWYQEKPGKAPKLLIYDASNLETGVPSRFSGSGSGTDFTFTISSLQ | |
| CODV | PEDIATYYCQQYYDLYTFGGGTKVEIKGGGGSGGGGSRTVAAPSVFIFP | |
| X a- | PSDEQLKSGTASVVCLLNNFYPREAKVQWKVDNALQSGNSQESVTEQD | |
| IL17A_VR- | SKDSTYSLSSTLTLSKADYEKHKVYACEVTHQGLSSPVTKSFNRGEC | |
| 92017-CM2)- | | |
| huIgG1- | | |
| CR3_NN3_LAL | | |
| A_DQ_dk Chain | | |
| 1 | | |
| (a-IL4_VR- | QLQLQESGPGLVKPSETLSLTCTVSGGSISSIPYYWGWIRKPPGKGLEWI | 143 |
| 93303 X a- | GSIYPSGSTDYNPSLKSRVTISVDTSKNQFSLKLSSVTAADTAVYYCARG | |
| IL13_VR- | GARDRIDHAFSIWGQGTMVTVSSEVQLLESGGGLVQPGGSLRLSCAAS | |
| 62822)-CM1- | GFTFSSYAMSWVRKAPGKGLEWVSAISGSGGSTYYADSVKGRFTISRDN | |
| CODV X a- | SKNTLYLQMNSLRAEDTAVYYCAREYYYDPLQYYHYYYGMDVWGQGTT | |
| IL17A_VR- | VTVSSASTKGPSVFPLAPSSKSTSGGTAALGCLVKDYFPEPVTVSWNSG | |
| 92017-CM2)- | ALTSGVHTFPAVLQSSGLYSLSSVVTVPSSSLGTQTYICNVNHKPSNTKV | |
| huIgG1- | DKKVEPKSCDKTHTCPPCPAPEAAGGPSVFLFPPKPKDTLMISRDPEVT | |
| CR3_NN3_LAL | CVVVDVSHEDPEVKFNWYVDGVEVHNAKTKPREEQYNSTYRVVSVLQV | |
| A_DQ_dk Chain | LHQDWLNGKEYKCKVSNKALPAPIEKTISKAKGQPREPQVYTLPPCRDEL | |
| 2 | TKNQVSLWCLVKGFYPSDIAVEWESNGQPENNYKTTPPVLDSDGSFFLY | |
| | SKLTVDKSRWQQGNVFSCSVMHEALHNHYTQKSLSLSPG | |
| (a-IL4_VR- | QVQLQQWGAGLLKPSETLSLTCAVYGGSFDGYYWNWIREPPGKGLEWIG | 144 |
| 93303 X a- | EINHIGGTIYNPSLKSRVTISEDTSKNQFSLKLSSVTAADTAVYYCARGI | |
| IL13_VR- | TMGRGNYYYYGLDVWGQGTTVTVSSASTKGPSVFPLAPSSKSTSGGTAA | |
| 62822)-CM1- | LGCLVKDYFPEPVTVSWNSGALTSGVHTFPAVLQSSGLYSLSSVVEVPS | |
| CODVX | SSLGTQTYICNVNHKPSNTKVDEKVEPDSCDKTHTCPPCPAPEAAGGPS | |
| a- | VFLFPPKPKDTLMISRDPEVTCVVVDVSHEDPEVKFNWYVDGVEVHNAK | |
| IL17A_VR- | TKPREEQYNSTYRVVSVLQVLHQDWLNGKEYKCKVSNKALPAPIEKTISK | |
| 92017-CM2)- | AKGQPREPQVCTLPPSRDELTKNQVSLSCAVKGFYPSDIAVEWESNGQP | |
| huIgG1- | ENNYKTTPPVLDSDGSFFLVSKLTVDKSRWQQGNVFSCSVMHEALHNR | |
| CR3_NN3_LAL | FTQKSLSLSPG | |
| A DQ_dk Chain | | |
| 3 | | |
| (a-IL4_VR- | DIQMTQSPSSLSASVGDRVTITCRASQDIKNDLGWYQKKPGKAPKFLIFG | 145 |
| 93303 X a- | TYSLQSGVPSRFSGSGSGTEFTLTISSLQPEDFATYYCLQDNNYPRTFG | |
| IL13_VR- | QGTKVEIKRTVAAPAVFIFPPSKKQLKSGTASVVCLLKNFYPREAKVQWK | |
| 62822)-CM1- | VDNALQSGNSQESVTEQDSKDSTYSLSSTLTLSKADYEKHKVYACEVTH | |
| CODV X a- | QGLSSPVTKSFNRGEC | |
| IL17A_VR- | | |
| 92017-CM2)- | | |
| huIgG1- | | |
| CR3_NN3_LAL | | |
| A_DQ_dk Chain | | |
| 4 | | |
| (a-IL4 VR- | SYELTQPPSVSVSPGQTARITCSGDALPKQYAYWYQEKPGQAPVLVIYK | 146 |
| 93303 X a- | DSERPSGIPERFSGSSKGTTVTLTISGVQAEDEADYYCQSADSSGTYWV | |
| IL13_VR- | FGGGTKLTVLGGGGSGGGGSDIQMTQSPSSLSASVGDRVTITCQASQDI | |
| 62822)-CM1- | SNYLNWYQEKPGKAPKLLIYDASNLETGVPSRFSGSGSGTDFTFTISSLQ | |
| CODV X a- | PEDIATYYCQQYYDLYTFGGGTKVEIKGGGGSGGGGSRTVAAPSVFIFP | |
| IL17A_VR- | PSDEQLKSGTASVVCLLNNFYPREAKVQWKVDNALQSGNSQESVTEQD | |
| 92018-CM2)- | SKDSTYSLSSTLTLSKADYEKHKVYACEVTHQGLSSPVTKSFNRGEC | |
| huIgG1- | | |
| CR3_NN3_LAL | | |
| A_DQ_dk Chain | | |
| 1 | | |
| (a-IL4_VR- | QLQLQESGPGLVKPSETLSLTCTVSGGSISSIPYYWGWIRKPPGKGLEWI | 147 |
| 93303 X a- | GSIYPSGSTDYNPSLKSRVTISVDTSKNQFSLKLSSVTAADTAVYYCARG | |
| IL13_VR- | GARDRIDHAFSIWGQGTMVTVSSEVQLLESGGGLVQPGGSLRLSCAAS | |
| 62822)-CM1- | GFTFSSYAMSWVRKAPGKGLEWVSAISGSGGSTYYADSVKGRFTISRDN | |
| CODV | SKNTLYLQMNSLRAEDTAVYYCAREYYYDPLQYYHYYYGMDVWGQGTT | |
| X a- | VTVSSASTKGPSVFPLAPSSKSTSGGTAALGCLVKDYFPEPVTVSWNSG | |
| IL17A_VR- | ALTSGVHTFPAVLQSSGLYSLSSVVTVPSSSLGTQTYICNVNHKPSNTKV | |
| 92018-CM2)- | DKKVEPKSCDKTHTCPPCPAPEAAGGPSVFLFPPKPKDTLMISRDPEVT | |
| huIgG1- | CVVVDVSHEDPEVKFNWYVDGVEVHNAKTKPREEQYNSTYRVVSVLQV | |
| CR3_NN3_LAL | LHQDWLNGKEYKCKVSNKALPAPIEKTISKAKGQPREPQVYTLPPCRDEL | |
| A_DQ_dk Chain | TKNQVSLWCLVKGFYPSDIAVEWESNGQPENNYKTTPPVLDSDGSFFLY | |
| 2 | SKLTVDKSRWQQGNVFSCSVMHEALHNHYTQKSLSLSPG | |
| (a-IL4_VR- | QVQLQQWGAGLLKPSETLSLTCAVFGGSFDGYYWNWIREPPGKGLEWI | 148 |
| 93303 X a- | GEIFHIGGTIYNPSLKSRVTISEDTSKNQFSLRLNSVTAADTATYYCARGIT | |
| IL13_VR- | MGRGNYYYYGLDVWGQGTPVTVSSASTKGPSVFPLAPSSKSTSGGTAA | |
| 62822)-CM1- | LGCLVKDYFPEPVTVSWNSGALTSGVHTFPAVLQSSGLYSLSSVVEVPS | |
| CODV X a- | SSLGTQTYICNVNHKPSNTKVDEKVEPDSCDKTHTCPPCPAPEAAGGPS | |
| IL17A_VR- | VFLFPPKPKDTLMISRDPEVTCVVVDVSHEDPEVKFNWYVDGVEVHNAK | |
| 92018-CM2)- | TKPREEQYNSTYRVVSVLQVLHQDWLNGKEYKCKVSNKALPAPIEKTISK | |
| huIgG1- | AKGQPREPQVCTLPPSRDELTKNQVSLSCAVKGFYPSDIAVEWESNGQP | |
| CR3_NN3_LAL | ENNYKTTPPVLDSDGSFFLVSKLTVDKSRWQQGNVFSCSVMHEALHNR | |
| A_DQ_dk Chain | FTQKSLSLSPG | |
| 3 | | |
| (a-IL4 VR- | DIQMTQSPSSLSASVGDRVTITCRASQDIKNDLGWYQKKPGKAPKFLIFG | 149 |
| 93303 X a- | TYSLQSGVPSRFSGSGSGTEFTLTISSLQPEDFATYYCLQDNNYPRTFG | |
| IL13_VR- | QGTKVEIKRTVAAPAVFIFPPSKKQLKSGTASVVCLLKNFYPREAKVQWK | |
| 62822)-CM1- | VDNALQSGNSQESVTEQDSKDSTYSLSSTLTLSKADYEKHKVYACEVTH | |
| CODV X a- | QGLSSPVTKSFNRGEC | |
| IL17A_VR- | | |
| 92018-CM2)- | | |
| huIgG1- | | |
| CR3_NN3_LAL | | |
| A_DQ_dk Chain | | |
| 4 | | |
| (a-IL4_VR- | SYELTQPPSVSVSPGQTARITCSGDALPKQYAYWYQEKPGQAPVLVIYK | 150 |
| 93303 X a- | DSERPSGIPERFSGSSKGTTVTLTISGVQAEDEADYYCQSADSSGTYWV | |
| IL13_VR- | FGGGTKLTVLGGGGSGGGGSDIQMTQSPSSLSASVGDRVTITCQASQDI | |
| 62822)-CM1- | SNYLNWYQEKPGKAPKLLIYDASNLETGVPSRFSGSGSGTDFTFTISSLQ | |
| CODV | PEDIATYYCQQYYDLYTFGGGTKVEIKGGGGSGGGGSRTVAAPSVFIFP | |
| X a- | PSDEQLKSGTASVVCLLNNFYPREAKVQWKVDNALQSGNSQESVTEQD | |
| IL17A_VR- | SKDSTYSLSSTLTLSKADYEKHKVYACEVTHQGLSSPVTKSFNRGEC | |
| 92019-CM2)- | | |
| huIgG1- | | |
| CR3_NN3_LAL | | |
| A_DQ_dk Chain | | |
| 1 | | |
| (a-IL4_VR- | QLQLQESGPGLVKPSETLSLTCTVSGGSISSIPYYWGWIRKPPGKGLEWI | 151 |
| 93303 X a- | GSIYPSGSTDYNPSLKSRVTISVDTSKNQFSLKLSSVTAADTAVYYCARG | |
| IL13_VR- | GARDRIDHAFSIWGQGTMVTVSSEVQLLESGGGLVQPGGSLRLSCAAS | |
| 62822)-CM1- | GFTFSSYAMSWVRKAPGKGLEWVSAISGSGGSTYYADSVKGRFTISRDN | |
| CODV X a- | SKNTLYLQMNSLRAEDTAVYYCAREYYYDPLQYYHYYYGMDVWGQGTT | |
| IL17A_VR- | VTVSSASTKGPSVFPLAPSSKSTSGGTAALGCLVKDYFPEPVTVSWNSG | |
| 92019-CM2)- | ALTSGVHTFPAVLQSSGLYSLSSVVTVPSSSLGTQTYICNVNHKPSNTKV | |
| huIgG1- | DKKVEPKSCDKTHTCPPCPAPEAAGGPSVFLFPPKPKDTLMISRDPEVT | |
| CR3_NN3_LAL | CVVVDVSHEDPEVKFNWYVDGVEVHNAKTKPREEQYNSTYRVVSVLQV | |
| A_DQ_dk Chain | LHQDWLNGKEYKCKVSNKALPAPIEKTISKAKGQPREPQVYTLPPCRDEL | |
| 2 | TKNQVSLWCLVKGFYPSDIAVEWESNGQPENNYKTTPPVLDSDGSFFLY | |
| | SKLTVDKSRWQQGNVFSCSVMHEALHNHYTQKSLSLSPG | |
| (a-IL4_VR- | EVQLVESGGGLVKPGGSLRLSCAASGFTFSDYFMSWIREAPGKGLEWV | 152 |
| 93303 X a- | SYISSTGTTKYYADSVKGRFTMSRDNAKNSLYLQMSSLRAEDTAVYYCA | |
| IL13_VR- | RDGSSLDSWGQGTLVTVSSASTKGPSVFPLAPSSKSTSGGTAALGCLVK | |
| 62822)-CM1- | DYFPEPVTVSWNSGALTSGVHTFPAVLQSSGLYSLSSVVEVPSSSLGTQ | |
| CODV | TYICNVNHKPSNTKVDEKVEPDSCDKTHTCPPCPAPEAAGGPSVFLFPP | |
| X a- | KPKDTLMISRDPEVTCVVVDVSHEDPEVKFNWYVDGVEVHNAKTKPREE | |
| IL17A_VR- | QYNSTYRVVSVLQVLHQDWLNGKEYKCKVSNKALPAPIEKTISKAKGQP | |
| 92019-CM2)- | REPQVCTLPPSRDELTKNQVSLSCAVKGFYPSDIAVEWESNGQPENNYK | |
| huIgG1- | TTPPVLDSDGSFFLVSKLTVDKSRWQQGNVFSCSVMHEALHNRFTQKSL | |
| CR3_NN3_LAL | SLSPG | |
| A_DQ_dk Chain | | |
| 3 | | |
| (a-IL4 VR- | DIVMTQSPLSLPVTPGEPASISCRSSQSLLDSDDGNTYLDWFLKKPGQSP | 153 |
| 93303 X a- | QLLIYTFFYRASGVPDRFSGSGSGTDFTLKISRVEAEDVGVYYCMQRIEF | |
| IL13_VR- | PYTFGQGTKLEIKRTVAAPAVFIFPPSKKQLKSGTASVVCLLKNFYPREAK | |
| 62822)-CM1- | VQWKVDNALQSGNSQESVTEQDSKDSTYSLSSTLTLSKADYEKHKVYAC | |
| CODV | EVTHQGLSSPVTKSFNRGEC | |
| X a- | | |
| IL17A_VR- | | |
| 92019-CM2)- | | |
| huIgG1- | | |
| CR3_NN3_LAL | | |
| A_DQ_dk Chain | | |
| 4 | | |
| (a-IL4_VR-62824 x a- | SYELTQPPSVSVSPGQTARITCSGDALPKQYAYWYQEKPGQAPVLVIYKDSERPSGIPERFS | 242 |
| IL13_VR-62822) x a- | GSSKGTTVTLTISGVQAEDEADYYCQSADSSGTYWVFGGGTKLTVLGGGGSGGGGSDIQ | |
| KLRK1_VR- | MTQSPSSLSASVGDRVTITCQASQDISNYLNWYQEKPGKAPKLLIYDASNLETGVPSRFSG | |
| 93305_huIgG1_LALA | SGSGTDFTFTISSLQPEDIATYYCQQYYDLYTFGGGTKVEIKGGGGSGGGGSRTVAAPSVFI | |
| _DQ_CR3_NN3_dk | FPPSDEQLKSGTASVVCLLNNFYPREAKVQWKVDNALQSGNSQESVTEQDSKDSTYSLSS | |
| chain 1 | TLTLSKADYEKHKVYACEVTHQGLSSPVTKSFNRGEC | |
| (a-IL4_VR-62824 x a- | QLQLQESGPGLVKPSETLSLTCTVSGGSISSESYYWGWIRKPPGKGLEWIGSIYYSGSTDYN | 243 |
| IL13_VR-62822) x a- | PSLKSRVTISVDTSKNQFSLKLSSVTAADTAVYYCARGGASDRIDHAFEIWGQGTMVTVSS | |
| KLRK1_VR- | EVQLLESGGGLVQPGGSLRLSCAASGFTFSSYAMSWVRKAPGKGLEWVSAISGSGGSTYY | |
| 93305_huIgG1_LALA | ADSVKGRFTISRDNSKNTLYLQMNSLRAEDTAVYYCAREYYYDPLQYYHYYYGMDVWGQ | |
| _DQ_CR3_NN3_dk | GTTVTVSSASTKGPSVFPLAPSSKSTSGGTAALGCLVKDYFPEPVTVSWNSGALTSGVHTF | |
| chain 2 | PAVLQSSGLYSLSSVVTVPSSSLGTQTYICNVNHKPSNTKVDKKVEPKSCDKTHTCPPCPAP | |
| | EAAGGPSVFLFPPKPKDTLMISRDPEVTCVVVDVSHEDPEVKFNWYVDGVEVHNAKTKP | |
| | REEQYNSTYRVVSVLQVLHQDWLNGKEYKCKVSNKALPAPIEKTISKAKGQPREPQVYTLP | |
| | PCRDELTKNQVSLWCLVKGFYPSDIAVEWESNGQPENNYKTTPPVLDSDGSFFLYSKLTV | |
| | DKSRWQQGNVFSCSVMHEALHNHYTQKSLSLSPG | |
| (a-IL4_VR-62824 x a- | QLQLQESGPGLVKPSETLSLTCTVSGGSISSSSYYWVWIRESPGKGLEWIGNIYSSGSTHYN | 244 |
| IL13_VR-62822) x a- | PSLKSRVIISVGTSKNQFSLKLKSVTAADTAVYYCAKPLLRDYYYYGVDVWGQGTTVTVSSA | |
| KLRK1_VR- | STKGPSVFPLAPSSKSTSGGTAALGCLVKDYFPEPVTVSWNSGALTSGVHTFPAVLQSSGL | |
| 93305_huIgG1_LALA | YSLSSVVEVPSSSLGTQTYICNVNHKPSNTKVDEKVEPDSCDKTHTCPPCPAPEAAGGPSV | |
| _DQ_CR3_NN3_dk | FLFPPKPKDTLMISRDPEVTCVVVDVSHEDPEVKFNWYVDGVEVHNAKTKPREEQYNSTY | |
| chain 3 | RVVSVLQVLHQDWLNGKEYKCKVSNKALPAPIEKTISKAKGQPREPQVCTLPPSRDELTKN | |
| | QVSLSCAVKGFYPSDIAVEWESNGQPENNYKTTPPVLDSDGSFFLVSKLTVDKSRWQQG | |
| | NVFSCSVMHEALHNRFTQKSLSLSPG | |
| (a-IL4_VR-62824 x a- | DIQLTPSPSFLSASVGDRVTITCRASQAISSYLAWYQKKPGKAPKLLISPASTLQSGVPSRFS | 245 |
| IL13_VR-62822) x a- | GSGSGTEFTLTISSLQPEDFATYFCQQLNSFPWTFGQGTKVEIKRTVAAPAVFIFPPSKKQL | |
| KLRK1_VR- | KSGTASVVCLLKNFYPREAKVQWKVDNALQSGNSQESVTEQDSKDSTYSLSSTLTLSKADY | |
| 93305_huIgG1_LALA | EKHKVYACEVTHQGLSSPVTKSFNRGEC | |
| _DQ_CR3_NN3_dk | | |
| chain 4 | | |
| (a-IL4_VR-93303 x a- | SYELTQPPSVSVSPGQTARITCSGDALPKQYAYWYQEKPGQAPVLVIYKDSERPSGIPERFS | 246 |
| IL13_VR-62822) x a- | GSSKGTTVTLTISGVQAEDEADYYCQSADSSGTYWVFGGGTKLTVLGGGGSGGGGSDIQ | |
| KLRK1_VR- | MTQSPSSLSASVGDRVTITCQASQDISNYLNWYQEKPGKAPKLLIYDASNLETGVPSRFSG | |
| 91186_huIgG1_LALA | SGSGTDFTFTISSLQPEDIATYYCQQYYDLYTFGGGTKVEIKGGGGSGGGGSRTVAAPSVFI | |
| _DQ_CR3_NN3_dk | FPPSDEQLKSGTASVVCLLNNFYPREAKVQWKVDNALQSGNSQESVTEQDSKDSTYSLSS | |
| chain 1 | TLTLSKADYEKHKVYACEVTHQGLSSPVTKSFNRGEC | |
| (a-IL4_VR-93303 x a- | QLQLQESGPGLVKPSETLSLTCTVSGGSISSIPYYWGWIRKPPGKGLEWIGSIYPSGSTDYN | 247 |
| IL13_VR-62822) x a- | PSLKSRVTISVDTSKNQFSLKLSSVTAADTAVYYCARGGARDRIDHAFSIWGQGTMVTVSS | |
| KLRK1_VR- | EVQLLESGGGLVQPGGSLRLSCAASGFTFSSYAMSWVRKAPGKGLEWVSAISGSGGSTYY | |
| 91186_huIgG1_LALA | ADSVKGRFTISRDNSKNTLYLQMNSLRAEDTAVYYCAREYYYDPLQYYHYYYGMDVWGQ | |
| _DQ_CR3_NN3_dk | GTTVTVSSASTKGPSVFPLAPSSKSTSGGTAALGCLVKDYFPEPVTVSWNSGALTSGVHTF | |
| chain 2 | PAVLQSSGLYSLSSVVTVPSSSLGTQTYICNVNHKPSNTKVDKKVEPKSCDKTHTCPPCPAP | |
| | EAAGGPSVFLFPPKPKDTLMISRDPEVTCVVVDVSHEDPEVKFNWYVDGVEVHNAKTKP | |
| | REEQYNSTYRVVSVLQVLHQDWLNGKEYKCKVSNKALPAPIEKTISKAKGQPREPQVYTLP | |
| | PCRDELTKNQVSLWCLVKGFYPSDIAVEWESNGQPENNYKTTPPVLDSDGSFFLYSKLTV | |
| | DKSRWQQGNVFSCSVMHEALHNHYTQKSLSLSPG | |
| (a-IL4_VR-93303 x a- | EVQLLESGGGLVQPGGSLRLSCTASGFTFSSYAMSWVREAPGKGLEWVSLISESGSSTYYA | 248 |
| IL13_VR-62822) x a- | ESVKGRFTISRDDSKNTLYLQMNSLRAEDTAVYYCAKDRWFEGYHSYYGMDVWGQGTT | |
| KLRK1_VR- | VTVSSASTKGPSVFPLAPSSKSTSGGTAALGCLVKDYFPEPVTVSWNSGALTSGVHTFPAV | |
| 91186_huIgG1_LALA | LQSSGLYSLSSVVEVPSSSLGTQTYICNVNHKPSNTKVDEKVEPDSCDKTHTCPPCPAPEAA | |
| _DQ_CR3_NN3_dk | GGPSVFLFPPKPKDTLMISRDPEVTCVVVDVSHEDPEVKFNWYVDGVEVHNAKTKPREE | |
| chain 3 | QYNSTYRVVSVLQVLHQDWLNGKEYKCKVSNKALPAPIEKTISKAKGQPREPQVCTLPPSR | |
| | DELTKNQVSLSCAVKGFYPSDIAVEWESNGQPENNYKTTPPVLDSDGSFFLVSKLTVDKSR | |
| | WQQGNVFSCSVMHEALHNRFTQKSLSLSPG | |
| (a-IL4_VR-93303 x a- | DIQLTQSPSFLSAFVGDRVTITCRASQGISNNLAWYQKKPGKAPKLLVYATSTLQSGVPSRF | 249 |
| IL13_VR-62822) x a- | SGSGSGTEFTLTVSSLQPEDFATYYCQQLKSYPSFTFGPGTKVDIKRTVAAPAVFIFPPSKKQ | |
| KLRK1_VR- | LKSGTASVVCLLKNFYPREAKVQWKVDNALQSGNSQESVTEQDSKDSTYSLSSTLTLSKAD | |
| 91186_huIgG1_LALA | YEKHKVYACEVTHQGLSSPVTKSFNRGEC | |
| _DQ_CR3_NN3_dk | | |
| chain 4 | | |
| (a-IL4_VR-82824 x a- | SYELTQPPSVSVSPGQTARITCSGDALPKQYAYWYQEKPGQAPVLVIYKDSERPSGIPERFS | 250 |
| IL13_VR-62822) x a- | GSSKGTTVTLTISGVQAEDEADYYCQSADSSGTYWVFGGGTKLTVLGGGGSGGGGSDIQ | |
| KLRK1_VR- | MTQSPSSLSASVGDRVTITCQASQDISNYLNWYQEKPGKAPKLLIYDASNLETGVPSRFSG | |
| 93304_huIgG1_LALA | SGSGTDFTFTISSLQPEDIATYYCQQYYDLYTFGGGTKVEIKGGGGSGGGGSRTVAAPSVFI | |
| _DQ_CR3_NN3_dk | FPPSDEQLKSGTASVVCLLNNFYPREAKVQWKVDNALQSGNSQESVTEQDSKDSTYSLSS | |
| chain 1 | TLTLSKADYEKHKVYACEVTHQGLSSPVTKSFNRGEC | |
| (a-IL4_VR-82824 x a- | QLQLQESGPGLVKPSETLSLTCTVSGGSISSESYYWGWIRKPPGKGLEWIGSIYYSGSTDYN | 251 |
| IL13_VR-62822) x a- | PSLKSRVTISVDTSKNQFSLKLSSVTAADTAVYYCARGGASDRIDHAFEIWGQGTMVTVSS | |
| KLRK1_VR- | EVQLLESGGGLVQPGGSLRLSCAASGFTFSSYAMSWVRKAPGKGLEWVSAISGSGGSTYY | |
| 93304_huIgG1_LALA | ADSVKGRFTISRDNSKNTLYLQMNSLRAEDTAVYYCAREYYYDPLQYYHYYYGMDVWGQ | |
| _DQ_CR3_NN3_dk | GTTVTVSSASTKGPSVFPLAPSSKSTSGGTAALGCLVKDYFPEPVTVSWNSGALTSGVHTF | |
| chain 2 | PAVLQSSGLYSLSSVVTVPSSSLGTQTYICNVNHKPSNTKVDKKVEPKSCDKTHTCPPCPAP | |
| | EAAGGPSVFLFPPKPKDTLMISRDPEVTCVVVDVSHEDPEVKFNWYVDGVEVHNAKTKP | |
| | REEQYNSTYRVVSVLQVLHQDWLNGKEYKCKVSNKALPAPIEKTISKAKGQPREPQVYTLP | |
| | PCRDELTKNQVSLWCLVKGFYPSDIAVEWESNGQPENNYKTTPPVLDSDGSFFLYSKLTV | |
| | DKSRWQQGNVFSCSVMHEALHNHYTQKSLSLSPG | |
| (a-IL4_VR-82824 x a- | EVQLLESGGGLVQPGGSLRLSCTASGFTFSSYAMSWVREAPGKGLEWVSLISESGSSTYYA | 252 |
| IL13_VR-62822) x a- | ESVKGRFTNSRDDSRNTLYLQMNSLRAEDTAVYYCAKDRWFEGYHSYYGMDVWGQGTT | |
| KLRK1_VR- | VTVSSASTKGPSVFPLAPSSKSTSGGTAALGCLVKDYFPEPVTVSWNSGALTSGVHTFPAV | |
| 93304_huIgG1_LALA | LQSSGLYSLSSVVEVPSSSLGTQTYICNVNHKPSNTKVDEKVEPDSCDKTHTCPPCPAPEAA | |
| _DQ_CR3_NN3_dk | GGPSVFLFPPKPKDTLMISRDPEVTCVVVDVSHEDPEVKFNWYVDGVEVHNAKTKPREE | |
| chain 3 | QYNSTYRVVSVLQVLHQDWLNGKEYKCKVSNKALPAPIEKTISKAKGQPREPQVCTLPPSR | |
| | DELTKNQVSLSCAVKGFYPSDIAVEWESNGQPENNYKTTPPVLDSDGSFFLVSKLTVDKSR | |
| | WQQGNVFSCSVMHEALHNRFTQKSLSLSPG | |
| (a-IL4_VR-82824 x a- | DIQLTQSPSFLSAFVGDRVTITCRASQGISNNLAWYQKKPGKAPKLLVYATSTLQSGVPSRF | 253 |
| IL13_VR-62822) x a- | SGSGSGTEFTLTVSSLQPEDFATYYCQQLKSYPSFTFGPGTKVDIKRTVAAPAVFIFPPSKKQ | |
| KLRK1_VR- | LKSGTASVVCLLKNFYPREAKVQWKVDNALQSGNSQESVTEQDSKDSTYSLSSTLTLSKAD | |
| 93304_huIgG1_LALA | YEKHKVYACEVTHQGLSSPVTKSFNRGEC | |
| _DQ_CR3_NN3_dK | | |
| chain 4 | | |

### Anti-IL-4 Binding Sites

Certain aspects of the present disclosure relate to binding proteins that comprise an antigen binding site that binds (e.g., specifically binds) an IL-4 polypeptide. In some embodiments, the IL-4 polypeptide is a human IL-4 polypeptide, also known as IL4, BSF1, BSF-1, BCGF1, and BCGF-1. Human IL-4 polypeptides are known in the art and include, without limitation, the polypeptide represented by NCBI Accession Number NP_000580.1, or a polypeptide produced from NCBI Gene ID Number 3565, which are incorporated herein by reference.

In some embodiments, a binding protein comprising an antigen binding site that binds an IL-4 polypeptide is monospecific and/or monovalent, bispecific and/or bivalent, trispecific and/or trivalent, or multispecific and/or multivalent.

In some embodiments, any of the CDRs and/or variable domains of the anti-IL-4 binding sites described below can be used in a monospecific antibody. In some embodiments, the monospecific antibody is a monoclonal antibody.

In some embodiments, any of the CDRs and/or variable domains of the anti-IL-4 binding sites described below can be used in a bispecific or multispecific antibody.

In some embodiments, any of the CDRs and/or variable domains of the anti-IL-4 binding sites described below (e.g., in Table 1) can be used in any binding site of a trispecific binding protein comprising four polypeptides that form three antigen binding sites, e.g., as described supra. In certain embodiments, a binding protein that comprises an antigen binding site that binds an IL-4 polypeptide is a trispecific binding protein comprising four polypeptides that form three antigen binding sites as described supra. In some embodiments, the V_{H1} and V_{L1} domains pair and form a first antigen binding site that binds an IL-4 polypeptide.

In some embodiments, a binding site that binds IL-4 comprises: an antibody heavy chain variable (VH) domain that comprises a CDR-H1 comprising the amino acid sequence of SEQ ID NO:1, a CDR-H2 comprising the amino acid sequence of SEQ ID NO:2, and a CDR-H3 comprising the amino acid sequence selected from the group consisting of SEQ ID NOs:3-5, and an antibody light chain variable (VH) domain that comprises a CDR-L1 comprising the amino acid sequence of SEQ ID NO:6 or 7, a CDR-L2 comprising the amino acid sequence of SEQ ID NO:8 or 9, and a CDR-L3 comprising the amino acid sequence of SEQ ID NO:10 or 11.

In some embodiments, a binding site that binds IL-4 comprises: a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3, and a VH domain that comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10.

In some embodiments, a binding site that binds IL-4 comprises: a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11.

In some embodiments, a binding site that binds IL-4 comprises: a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:4, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11.

In some embodiments, a binding site that binds IL-4 comprises: a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:5, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11.

In some embodiments, a binding site that binds IL-4 comprises: an antibody heavy chain variable (VH) domain that comprises a CDR-H1 comprising the amino acid sequence of SEQ ID NO: 12, a CDR-H2 comprising the amino acid sequence of SEQ ID NO:13, and a CDR-H3 comprising the amino acid sequence selected from the group consisting of SEQ ID NOs:14-16, and an antibody light chain variable (VH) domain that comprises a CDR-L1 comprising the amino acid sequence of SEQ ID NO: 17 or 18, a CDR-L2 comprising the amino acid sequence of DAS, and a CDR-L3 comprising the amino acid sequence of SEQ ID NO:20 or 21.

In some embodiments, a binding site that binds IL-4 comprises: a VH domain that comprises a CDR-H1 comprising the amino acid sequence of SEQ ID NO:12, a CDR-H2 comprising the amino acid sequence of SEQ ID NO: 13, and a CDR-H3 comprising the amino acid sequence of SEQ ID NO:14, and a VH domain that comprises a CDR-L1 comprising the amino acid sequence of SEQ ID NO:17, a CDR-L2 comprising the amino acid sequence of DAS, and a CDR-L3 comprising the amino acid sequence of SEQ ID NO:20.

In some embodiments, a binding site that binds IL-4 comprises: a VH domain that comprises a CDR-H1 comprising the amino acid sequence of SEQ ID NO:12, a CDR-H2 comprising the amino acid sequence of SEQ ID NO: 13, and a CDR-H3 comprising the amino acid sequence of SEQ ID NO:14, and a VH domain that comprises a CDR-L1 comprising the amino acid sequence of SEQ ID NO:18, a CDR-L2 comprising the amino acid sequence of DAS, and a CDR-L3 comprising the amino acid sequence of SEQ ID NO:21.

In some embodiments, a binding site that binds IL-4 comprises: a VH domain that comprises a CDR-H1 comprising the amino acid sequence of SEQ ID NO:12, a CDR-H2 comprising the amino acid sequence of SEQ ID NO: 13, and a CDR-H3 comprising the amino acid sequence of SEQ ID NO:15, and a VH domain that comprises a CDR-L1 comprising the amino acid sequence of SEQ ID NO:18, a CDR-L2 comprising the amino acid sequence of DAS, and a CDR-L3 comprising the amino acid sequence of SEQ ID NO:21.

In some embodiments, a binding site that binds IL-4 comprises: a VH domain that comprises a CDR-H1 comprising the amino acid sequence of SEQ ID NO:12, a CDR-H2 comprising the amino acid sequence of SEQ ID NO: 13, and a CDR-H3 comprising the amino acid sequence of SEQ ID NO:16, and a VH domain that comprises a CDR-L1 comprising the amino acid sequence of SEQ ID NO:18, a CDR-L2 comprising the amino acid sequence of DAS, and a CDR-L3 comprising the amino acid sequence of SEQ ID NO:21.

In some embodiments, a binding site that binds IL-4 comprises: a VH domain that comprises an amino acid sequence that is at least 95%, at least 96%, at least 97%, at least 98%, at least 99%, or 100% identical to the amino acid sequence of SEQ ID NO:34 and/or a VL domain that comprises an amino acid sequence that is at least 95%, at least 96%, at least 97%, at least 98%, at least 99%, or 100% identical to the amino acid sequence of SEQ ID NO:35. In some embodiments, a binding site that binds IL-4 comprises: a VH domain that comprises the amino acid sequence of SEQ ID NO:34 and/or a VH domain that comprises the amino acid sequence of SEQ ID NO:35. In some embodiments, a binding site that binds IL-4 comprises: a VH domain that comprises the amino acid sequence of SEQ ID NO:34 and a VL domain that comprises the amino acid sequence of SEQ ID NO:35. In some embodiments, a binding site that binds IL-4 comprises: a VH domain that comprises 1, 2, or all 3 CDRs from a VH domain comprising the amino acid sequence of SEQ ID NO:34 and/or a VH domain that comprises 1, 2, or all 3 CDRs from a VH domain comprising the amino acid sequence of SEQ ID NO:35.

In some embodiments, a binding site that binds IL-4 comprises: a VH domain that comprises an amino acid sequence that is at least 95%, at least 96%, at least 97%, at least 98%, at least 99%, or 100% identical to the amino acid sequence of SEQ ID NO:36 and/or a VL domain that comprises an amino acid sequence that is at least 95%, at least 96%, at least 97%, at least 98%, at least 99%, or 100% identical to the amino acid sequence of SEQ ID NO:37. In some embodiments, a binding site that binds IL-4 comprises: a VH domain that comprises the amino acid sequence of SEQ ID NO:36 and/or a VH domain that comprises the amino acid sequence of SEQ ID NO:37. In some embodiments, a binding site that binds IL-4 comprises: a VH domain that comprises the amino acid sequence of SEQ ID NO:36 and a VL domain that comprises the amino acid sequence of SEQ ID NO:37. In some embodiments, a binding site that binds IL-4 comprises: a VH domain that comprises 1, 2, or all 3 CDRs from a VH domain comprising the amino acid sequence of SEQ ID NO:36 and/or a VH domain that comprises 1, 2, or all 3 CDRs from a VH domain comprising the amino acid sequence of SEQ ID NO:37.

In some embodiments, a binding site that binds IL-4 comprises: a VH domain that comprises an amino acid sequence that is at least 95%, at least 96%, at least 97%, at least 98%, at least 99%, or 100% identical to the amino acid sequence of SEQ ID NO:38 and/or a VL domain that comprises an amino acid sequence that is at least 95%, at least 96%, at least 97%, at least 98%, at least 99%, or 100% identical to the amino acid sequence of SEQ ID NO:39. In some embodiments, a binding site that binds IL-4 comprises: a VH domain that comprises the amino acid sequence of SEQ ID NO:38 and/or a VH domain that comprises the amino acid sequence of SEQ ID NO:39. In some embodiments, a binding site that binds IL-4 comprises: a VH domain that comprises the amino acid sequence of SEQ ID NO:38 and a VL domain that comprises the amino acid sequence of SEQ ID NO:39. In some embodiments, a binding site that binds IL-4 comprises: a VH domain that comprises 1, 2, or all 3 CDRs from a VH domain comprising the amino acid sequence of SEQ ID NO:38 and/or a VH domain that comprises 1, 2, or all 3 CDRs from a VH domain comprising the amino acid sequence of SEQ ID NO:39.

In some embodiments, a binding site that binds IL-4 comprises: a VH domain that comprises an amino acid sequence that is at least 95%, at least 96%, at least 97%, at least 98%, at least 99%, or 100% identical to the amino acid sequence of SEQ ID NO:40 and/or a V_{L} domain that comprises an amino acid sequence that is at least 95%, at least 96%, at least 97%, at least 98%, at least 99%, or 100% identical to the amino acid sequence of SEQ ID NO:41. In some embodiments, a binding site that binds IL-4 comprises: a VH domain that comprises the amino acid sequence of SEQ ID NO:40 and/or a VH domain that comprises the amino acid sequence of SEQ ID NO:41. In some embodiments, a binding site that binds IL-4 comprises: a VH domain that comprises the amino acid sequence of SEQ ID NO:40 and a VL domain that comprises the amino acid sequence of SEQ ID NO:41. In some embodiments, a binding site that binds IL-4 comprises: a VH domain that comprises 1, 2, or all 3 CDRs from a VH domain comprising the amino acid sequence of SEQ ID NO:40 and/or a VH domain that comprises 1, 2, or all 3 CDRs from a VH domain comprising the amino acid sequence of SEQ ID NO:41.

In some embodiments, a binding site that binds IL-4 comprises: a VH domain that comprises an amino acid sequence that is at least 95%, at least 96%, at least 97%, at least 98%, at least 99%, or 100% identical to the amino acid sequence of SEQ ID NO:42 and/or a V_{L} domain that comprises an amino acid sequence that is at least 95%, at least 96%, at least 97%, at least 98%, at least 99%, or 100% identical to the amino acid sequence of SEQ ID NO:43. In some embodiments, a binding site that binds IL-4 comprises: a VH domain that comprises the amino acid sequence of SEQ ID NO:42 and/or a VH domain that comprises the amino acid sequence of SEQ ID NO:43. In some embodiments, a binding site that binds IL-4 comprises: a VH domain that comprises the amino acid sequence of SEQ ID NO:42 and a VL domain that comprises the amino acid sequence of SEQ ID NO:43. In some embodiments, a binding site that binds IL-4 comprises: a VH domain that comprises 1, 2, or all 3 CDRs from a VH domain comprising the amino acid sequence of SEQ ID NO:42 and/or a VH domain that comprises 1, 2, or all 3 CDRs from a VH domain comprising the amino acid sequence of SEQ ID NO:43.

In some embodiments, a binding site that binds IL-4 comprises: a VH domain that comprises an amino acid sequence that is at least 95%, at least 96%, at least 97%, at least 98%, at least 99%, or 100% identical to the amino acid sequence of SEQ ID NO:44 and/or a V_{L} domain that comprises an amino acid sequence that is at least 95%, at least 96%, at least 97%, at least 98%, at least 99%, or 100% identical to the amino acid sequence of SEQ ID NO:43. In some embodiments, a binding site that binds IL-4 comprises: a VH domain that comprises the amino acid sequence of SEQ ID NO:44 and/or a VH domain that comprises the amino acid sequence of SEQ ID NO:43. In some embodiments, a binding site that binds IL-4 comprises: a VH domain that comprises the amino acid sequence of SEQ ID NO:44 and a VL domain that comprises the amino acid sequence of SEQ ID NO:43. In some embodiments, a binding site that binds IL-4 comprises: a VH domain that comprises 1, 2, or all 3 CDRs from a VH domain comprising the amino acid sequence of SEQ ID NO:44 and/or a VH domain that comprises 1, 2, or all 3 CDRs from a VH domain comprising the amino acid sequence of SEQ ID NO:43.

In some embodiments, a binding site that binds IL-4 comprises: a VH domain that comprises an amino acid sequence that is at least 95%, at least 96%, at least 97%, at least 98%, at least 99%, or 100% identical to the amino acid sequence of SEQ ID NO:45 and/or a V_{L} domain that comprises an amino acid sequence that is at least 95%, at least 96%, at least 97%, at least 98%, at least 99%, or 100% identical to the amino acid sequence of SEQ ID NO:43. In some embodiments, a binding site that binds IL-4 comprises: a VH domain that comprises the amino acid sequence of SEQ ID NO:45 and/or a VH domain that comprises the amino acid sequence of SEQ ID NO:43. In some embodiments, a binding site that binds IL-4 comprises: a VH domain that comprises the amino acid sequence of SEQ ID NO:45 and a VL domain that comprises the amino acid sequence of SEQ ID NO:43. In some embodiments, a binding site that binds IL-4 comprises: a VH domain that comprises 1, 2, or all 3 CDRs from a VH domain comprising the amino acid sequence of SEQ ID NO:45 and/or a VH domain that comprises 1, 2, or all 3 CDRs from a VH domain comprising the amino acid sequence of SEQ ID NO:43.

In some embodiments, a binding site that binds IL-4 comprises: an antibody heavy chain variable (VH) domain that comprises a CDR-H1 comprising the amino acid sequence of SEQ ID NO:22, a CDR-H2 comprising the amino acid sequence of SEQ ID NO:23, and a CDR-H3 comprising the amino acid sequence selected from the group consisting of SEQ ID NOs:24-27, and an antibody light chain variable (VH) domain that comprises a CDR-L1 comprising the amino acid sequence of SEQ ID NO:6 or 7, a CDR-L2 comprising the amino acid sequence of SEQ ID NO:8 or 9, and a CDR-L3 comprising the amino acid sequence of SEQ ID NO:10 or 11.

In some embodiments, a binding site that binds IL-4 comprises: a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24, and a VH domain that comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10.

In some embodiments, a binding site that binds IL-4 comprises: a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11.

In some embodiments, a binding site that binds IL-4 comprises: a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, and a VH domain that comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10.

In some embodiments, a binding site that binds IL-4 comprises: a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11.

In some embodiments, a binding site that binds IL-4 comprises: a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:26, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11.

In some embodiments, a binding site that binds IL-4 comprises: a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:27, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11.

In some embodiments, a binding site that binds IL-4 comprises: an antibody heavy chain variable (VH) domain that comprises a CDR-H1 comprising the amino acid sequence of SEQ ID NO:28, a CDR-H2 comprising the amino acid sequence of SEQ ID NO:29, and a CDR-H3 comprising the amino acid sequence selected from the group consisting of SEQ ID NOs:30-33, and an antibody light chain variable (VH) domain that comprises a CDR-L1 comprising the amino acid sequence of SEQ ID NO: 17 or 18, a CDR-L2 comprising the amino acid sequence of DAS, and a CDR-L3 comprising the amino acid sequence of SEQ ID NO:20 or 21.

In some embodiments, a binding site that binds IL-4 comprises: a VH domain that comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:30, and a VH domain that comprises a CDR-L1 comprising the amino acid sequence of SEQ ID NO:17, a CDR-L2 comprising the amino acid sequence of DAS, and a CDR-L3 comprising the amino acid sequence of SEQ ID NO:20.

In some embodiments, a binding site that binds IL-4 comprises: a VH domain that comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:30, and a VH domain that comprises a CDR-L1 comprising the amino acid sequence of SEQ ID NO:18, a CDR-L2 comprising the amino acid sequence of DAS, and a CDR-L3 comprising the amino acid sequence of SEQ ID NO:21.

In some embodiments, a binding site that binds IL-4 comprises: a VH domain that comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:31, and a VH domain that comprises a CDR-L1 comprising the amino acid sequence of SEQ ID NO:17, a CDR-L2 comprising the amino acid sequence of DAS, and a CDR-L3 comprising the amino acid sequence of SEQ ID NO:20.

In some embodiments, a binding site that binds IL-4 comprises: a VH domain that comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:31, and a VH domain that comprises a CDR-L1 comprising the amino acid sequence of SEQ ID NO:18, a CDR-L2 comprising the amino acid sequence of DAS, and a CDR-L3 comprising the amino acid sequence of SEQ ID NO:21.

In some embodiments, a binding site that binds IL-4 comprises: a VH domain that comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:32, and a VH domain that comprises a CDR-L1 comprising the amino acid sequence of SEQ ID NO:18, a CDR-L2 comprising the amino acid sequence of DAS, and a CDR-L3 comprising the amino acid sequence of SEQ ID NO:21.

In some embodiments, a binding site that binds IL-4 comprises: a VH domain that comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:33, and a VH domain that comprises a CDR-L1 comprising the amino acid sequence of SEQ ID NO:18, a CDR-L2 comprising the amino acid sequence of DAS, and a CDR-L3 comprising the amino acid sequence of SEQ ID NO:21.

In some embodiments, a binding site that binds IL-4 comprises: a VH domain that comprises an amino acid sequence that is at least 95%, at least 96%, at least 97%, at least 98%, at least 99%, or 100% identical to the amino acid sequence of SEQ ID NO:46 and/or a V_{L} domain that comprises an amino acid sequence that is at least 95%, at least 96%, at least 97%, at least 98%, at least 99%, or 100% identical to the amino acid sequence of SEQ ID NO:47. In some embodiments, a binding site that binds IL-4 comprises: a VH domain that comprises the amino acid sequence of SEQ ID NO:46 and/or a VH domain that comprises the amino acid sequence of SEQ ID NO:47. In some embodiments, a binding site that binds IL-4 comprises: a VH domain that comprises the amino acid sequence of SEQ ID NO:46 and a VL domain that comprises the amino acid sequence of SEQ ID NO:47. In some embodiments, a binding site that binds IL-4 comprises: a VH domain that comprises 1, 2, or all 3 CDRs from a VH domain comprising the amino acid sequence of SEQ ID NO:46 and/or a VH domain that comprises 1, 2, or all 3 CDRs from a VH domain comprising the amino acid sequence of SEQ ID NO:47.

In some embodiments, a binding site that binds IL-4 comprises: a VH domain that comprises an amino acid sequence that is at least 95%, at least 96%, at least 97%, at least 98%, at least 99%, or 100% identical to the amino acid sequence of SEQ ID NO:48 and/or a V_{L} domain that comprises an amino acid sequence that is at least 95%, at least 96%, at least 97%, at least 98%, at least 99%, or 100% identical to the amino acid sequence of SEQ ID NO:39. In some embodiments, a binding site that binds IL-4 comprises: a VH domain that comprises the amino acid sequence of SEQ ID NO:48 and/or a VH domain that comprises the amino acid sequence of SEQ ID NO:39. In some embodiments, a binding site that binds IL-4 comprises: a VH domain that comprises the amino acid sequence of SEQ ID NO:48 and a VL domain that comprises the amino acid sequence of SEQ ID NO:39. In some embodiments, a binding site that binds IL-4 comprises: a VH domain that comprises 1, 2, or all 3 CDRs from a VH domain comprising the amino acid sequence of SEQ ID NO:48 and/or a VH domain that comprises 1, 2, or all 3 CDRs from a VH domain comprising the amino acid sequence of SEQ ID NO:39.

In some embodiments, a binding site that binds IL-4 comprises: a VH domain that comprises an amino acid sequence that is at least 95%, at least 96%, at least 97%, at least 98%, at least 99%, or 100% identical to the amino acid sequence of SEQ ID NO:49 and/or a V_{L} domain that comprises an amino acid sequence that is at least 95%, at least 96%, at least 97%, at least 98%, at least 99%, or 100% identical to the amino acid sequence of SEQ ID NO:41. In some embodiments, a binding site that binds IL-4 comprises: a VH domain that comprises the amino acid sequence of SEQ ID NO:49 and/or a VH domain that comprises the amino acid sequence of SEQ ID NO:41. In some embodiments, a binding site that binds IL-4 comprises: a VH domain that comprises the amino acid sequence of SEQ ID NO:49 and a VL domain that comprises the amino acid sequence of SEQ ID NO:41. In some embodiments, a binding site that binds IL-4 comprises: a VH domain that comprises 1, 2, or all 3 CDRs from a VH domain comprising the amino acid sequence of SEQ ID NO:49 and/or a VH domain that comprises 1, 2, or all 3 CDRs from a VH domain comprising the amino acid sequence of SEQ ID NO:41.

In some embodiments, a binding site that binds IL-4 comprises: a VH domain that comprises an amino acid sequence that is at least 95%, at least 96%, at least 97%, at least 98%, at least 99%, or 100% identical to the amino acid sequence of SEQ ID NO:50 and/or a V_{L} domain that comprises an amino acid sequence that is at least 95%, at least 96%, at least 97%, at least 98%, at least 99%, or 100% identical to the amino acid sequence of SEQ ID NO:43. In some embodiments, a binding site that binds IL-4 comprises: a VH domain that comprises the amino acid sequence of SEQ ID NO:50 and/or a VH domain that comprises the amino acid sequence of SEQ ID NO:43. In some embodiments, a binding site that binds IL-4 comprises: a VH domain that comprises the amino acid sequence of SEQ ID NO:50 and a VL domain that comprises the amino acid sequence of SEQ ID NO:43. In some embodiments, a binding site that binds IL-4 comprises: a VH domain that comprises 1, 2, or all 3 CDRs from a VH domain comprising the amino acid sequence of SEQ ID NO:50 and/or a VH domain that comprises 1, 2, or all 3 CDRs from a VH domain comprising the amino acid sequence of SEQ ID NO:43.

In some embodiments, a binding site that binds IL-4 comprises: a VH domain that comprises an amino acid sequence that is at least 95%, at least 96%, at least 97%, at least 98%, at least 99%, or 100% identical to the amino acid sequence of SEQ ID NO:51 and/or a V_{L} domain that comprises an amino acid sequence that is at least 95%, at least 96%, at least 97%, at least 98%, at least 99%, or 100% identical to the amino acid sequence of SEQ ID NO:37. In some embodiments, a binding site that binds IL-4 comprises: a VH domain that comprises the amino acid sequence of SEQ ID NO:51 and/or a VH domain that comprises the amino acid sequence of SEQ ID NO:37. In some embodiments, a binding site that binds IL-4 comprises: a VH domain that comprises the amino acid sequence of SEQ ID NO:51 and a VL domain that comprises the amino acid sequence of SEQ ID NO:37. In some embodiments, a binding site that binds IL-4 comprises: a VH domain that comprises 1, 2, or all 3 CDRs from a VH domain comprising the amino acid sequence of SEQ ID NO:51 and/or a VH domain that comprises 1, 2, or all 3 CDRs from a VH domain comprising the amino acid sequence of SEQ ID NO:37.

In some embodiments, a binding site that binds IL-4 comprises: a VH domain that comprises an amino acid sequence that is at least 95%, at least 96%, at least 97%, at least 98%, at least 99%, or 100% identical to the amino acid sequence of SEQ ID NO:52 and/or a V_{L} domain that comprises an amino acid sequence that is at least 95%, at least 96%, at least 97%, at least 98%, at least 99%, or 100% identical to the amino acid sequence of SEQ ID NO:39. In some embodiments, a binding site that binds IL-4 comprises: a VH domain that comprises the amino acid sequence of SEQ ID NO:52 and/or a VH domain that comprises the amino acid sequence of SEQ ID NO:39. In some embodiments, a binding site that binds IL-4 comprises: a VH domain that comprises the amino acid sequence of SEQ ID NO:52 and a VL domain that comprises the amino acid sequence of SEQ ID NO:39. In some embodiments, a binding site that binds IL-4 comprises: a VH domain that comprises 1, 2, or all 3 CDRs from a VH domain comprising the amino acid sequence of SEQ ID NO:52 and/or a VH domain that comprises 1, 2, or all 3 CDRs from a VH domain comprising the amino acid sequence of SEQ ID NO:39.

In some embodiments, a binding site that binds IL-4 comprises: a VH domain that comprises an amino acid sequence that is at least 95%, at least 96%, at least 97%, at least 98%, at least 99%, or 100% identical to the amino acid sequence of SEQ ID NO:53 and/or a V_{L} domain that comprises an amino acid sequence that is at least 95%, at least 96%, at least 97%, at least 98%, at least 99%, or 100% identical to the amino acid sequence of SEQ ID NO:41. In some embodiments, a binding site that binds IL-4 comprises: a VH domain that comprises the amino acid sequence of SEQ ID NO:53 and/or a VH domain that comprises the amino acid sequence of SEQ ID NO:41. In some embodiments, a binding site that binds IL-4 comprises: a VH domain that comprises the amino acid sequence of SEQ ID NO:53 and a VL domain that comprises the amino acid sequence of SEQ ID NO:41. In some embodiments, a binding site that binds IL-4 comprises: a VH domain that comprises 1, 2, or all 3 CDRs from a VH domain comprising the amino acid sequence of SEQ ID NO:53 and/or a VH domain that comprises 1, 2, or all 3 CDRs from a VH domain comprising the amino acid sequence of SEQ ID NO:41.

In some embodiments, a binding site that binds IL-4 comprises: a VH domain that comprises an amino acid sequence that is at least 95%, at least 96%, at least 97%, at least 98%, at least 99%, or 100% identical to the amino acid sequence of SEQ ID NO:54 and/or a V_{L} domain that comprises an amino acid sequence that is at least 95%, at least 96%, at least 97%, at least 98%, at least 99%, or 100% identical to the amino acid sequence of SEQ ID NO:43. In some embodiments, a binding site that binds IL-4 comprises: a VH domain that comprises the amino acid sequence of SEQ ID NO:54 and/or a VH domain that comprises the amino acid sequence of SEQ ID NO:43. In some embodiments, a binding site that binds IL-4 comprises: a VH domain that comprises the amino acid sequence of SEQ ID NO:54 and a VL domain that comprises the amino acid sequence of SEQ ID NO:43. In some embodiments, a binding site that binds IL-4 comprises: a VH domain that comprises 1, 2, or all 3 CDRs from a VH domain comprising the amino acid sequence of SEQ ID NO:54 and/or a VH domain that comprises 1, 2, or all 3 CDRs from a VH domain comprising the amino acid sequence of SEQ ID NO:43.

In some embodiments, a binding site that binds IL-4 comprises: a VH domain that comprises an amino acid sequence that is at least 95%, at least 96%, at least 97%, at least 98%, at least 99%, or 100% identical to the amino acid sequence of SEQ ID NO:55 and/or a V_{L} domain that comprises an amino acid sequence that is at least 95%, at least 96%, at least 97%, at least 98%, at least 99%, or 100% identical to the amino acid sequence of SEQ ID NO:43. In some embodiments, a binding site that binds IL-4 comprises: a VH domain that comprises the amino acid sequence of SEQ ID NO:55 and/or a VH domain that comprises the amino acid sequence of SEQ ID NO:43. In some embodiments, a binding site that binds IL-4 comprises: a VH domain that comprises the amino acid sequence of SEQ ID NO:55 and a VL domain that comprises the amino acid sequence of SEQ ID NO:43. In some embodiments, a binding site that binds IL-4 comprises: a VH domain that comprises 1, 2, or all 3 CDRs from a VH domain comprising the amino acid sequence of SEQ ID NO:55 and/or a VH domain that comprises 1, 2, or all 3 CDRs from a VH domain comprising the amino acid sequence of SEQ ID NO:43.

In some embodiments, a binding site that binds IL-4 comprises: a VH domain that comprises an amino acid sequence that is at least 95%, at least 96%, at least 97%, at least 98%, at least 99%, or 100% identical to the amino acid sequence of SEQ ID NO:56 and/or a V_{L} domain that comprises an amino acid sequence that is at least 95%, at least 96%, at least 97%, at least 98%, at least 99%, or 100% identical to the amino acid sequence of SEQ ID NO:43. In some embodiments, a binding site that binds IL-4 comprises: a VH domain that comprises the amino acid sequence of SEQ ID NO:56 and/or a VH domain that comprises the amino acid sequence of SEQ ID NO:43. In some embodiments, a binding site that binds IL-4 comprises: a VH domain that comprises the amino acid sequence of SEQ ID NO:56 and a VL domain that comprises the amino acid sequence of SEQ ID NO:43. In some embodiments, a binding site that binds IL-4 comprises: a VH domain that comprises 1, 2, or all 3 CDRs from a VH domain comprising the amino acid sequence of SEQ ID NO:56 and/or a VH domain that comprises 1, 2, or all 3 CDRs from a VH domain comprising the amino acid sequence of SEQ ID NO:43.

Sequences of exemplary anti-IL-4 antigen binding sites are provided in Table 1. In some embodiments, a binding protein comprising an anti-IL-4 antigen binding site of the present disclosure comprises 1, 2, 3, 4, 5, or all 6 CDR sequences of an anti-IL-4 antibody described in Table 1. In some embodiments, a binding protein comprising an anti-IL-4 antigen binding site of the present disclosure comprises a VH domain sequence and/or VL domain sequence of an anti-IL-4 antibody described in Table 1.

TABLE 1
Anti-IL-4 binding protein sequences.

| Description | Sequence | SEQ ID NO |
|---|---|---|
| ADI-46659 | SIPYYWG | 1 |
| D111S CDR-H1 | | |
| (Kabat) | | |
| ADI-46659 | SIYPSGSTDYNPSLKS | 2 |
| D111S CDR-H2 | | |
| (Kabat) | | |
| ADI-46659 | GGARDRIDHAFSI | 3 |
| D111S CDR-H3 | | |
| (Kabat) | | |
| ADI-46659 | GGARDRIDHAFEI | 4 |
| D111E CDR-H3 | | |
| (Kabat) | | |
| ADI-46659 no | GGARDRIDHAFDI | 5 |
| D111S/E CDR- | | |
| H3 (Kabat) | | |
| ADI-46659 | QASQSISNYLN | 6 |
| D111S CDR-L1 | | |
| (Kabat) | | |
| ADI-46659 | QASQDISNYLN | 7 |
| D111S no V2 | | |
| CDR-L1 (Kabat) | | |
| ADI-46659 | DASNSET | 8 |
| D111S CDR-L2 | | |
| (Kabat) | | |
| ADI-46659 | DASNLET | 9 |
| D111S no V2 | | |
| CDR-L2 (Kabat) | | |
| ADI-46659 | QQYYDLYK | 10 |
| D111S CDR-L3 | | |
| (Kabat) | | |
| ADI-46659 | QQYYDLYT | 11 |
| D111S no V2 | | |
| CDR-L3 (Kabat) | | |
| ADI-46659 | GGSISSIPYY | 12 |
| D111S CDR-H1 | | |
| (IMGT) | | |
| ADI-46659 | IYPSGST | 13 |
| D111S CDR-H2 | | |
| (IMGT) | | |
| ADI-46659 | ARGGARDRIDHAFSI | 14 |
| D111S CDR-H3 | | |
| (IMGT) | | |
| ADI-46659 | ARGGARDRIDHAFEI | 15 |
| D111E CDR-H3 | | |
| (IMGT) | | |
| ADI-46659 no | ARGGARDRIDHAFDI | 16 |
| D111S/E CDR- | | |
| H3 (IMGT) | | |
| ADI-46659 | QSISNY | 17 |
| D111S CDR-L1 | | |
| (IMGT) | | |
| ADI-46659 | QDISNY | 18 |
| D111S no V2 | | |
| CDR-L1 (IMGT) | | |
| ADI-46659 | DAS | 19 |
| D111S CDR-L2 | | |
| (IMGT) | | |
| ADI-46659 | QQYYDLYK | 20 |
| D111S CDR-L3 | | |
| (IMGT) | | |
| ADI-46659 | QQYYDLYT | 21 |
| D111S no V2 | | |
| CDR-L3 (IMGT) | | |
| ADI-46653 | SESYYWG | 22 |
| CDR-H1 (Kabat) | | |
| ADI-46653 | SIYYSGSTDYNPSLKS | 23 |
| CDR-H2 (Kabat) | | |
| ADI-46653 | GGASDRIDKAFEI | 24 |
| CDR-H3 (Kabat) | | |
| H108K, D111E | | |
| ADI-46653 | GGASDRIDHAFEI | 25 |
| D111E CDR-H3 | | |
| (Kabat) | | |
| ADI-46653 | GGASDRIDHAFSI | 26 |
| D111S CDR-H3 | | |
| (Kabat) | | |
| ADI-46653 no | GGASDRIDHAFDI | 27 |
| D111S/E CDR- | | |
| H3 (Kabat) | | |
| ADI-46653 | GGSISSESYY | 28 |
| CDR-H1 (IMGT) | | |
| ADI-46653 | IYYSGST | 29 |
| CDR-H2 (IMGT) | | |
| ADI-46653 | ARGGASDRIDKAFEI | 30 |
| CDR-H3 (IMGT) | | |
| H108K, D111E | | |
| ADI-46653 | ARGGASDRIDHAFEI | 31 |
| D111E CDR-H3 | | |
| (IMGT) | | |
| ADI-46653 | ARGGASDRIDHAFSI | 32 |
| D111S CDR-H3 | | |
| (IMGT) | | |
| ADI-46653 no | ARGGASDRIDHAFDI | 33 |
| D111S/E CDR- | | |
| H3 (IMGT) | | |
| ADI-46659- | QLQLQESGPGLKKPSETLSLTCTVSGGSISSIPYYWGWIREPPGKGLEWIGS | 34 |
| D111S (V2)- | IYPSGSTDYNPSLKSRVTISVDTSKNQFSLKLSSVTAADTAVYYCARGGARD | |
| CM2_VH | RIDHAFSIWGQGTMVTVSS | |
| ADI-46659- | DIQMTQSPSSLSASVGDRVTITCQASQSISNYLNWYQKKPGKAPKLLIYDA | 35 |
| D111S (V2)- | SNSETGVPSRFSGSGSGTDFTFTISSLQPEDIATYYCQQYYDLYKFGGGTKV | |
| CM2_VL | EIK | |
| ADI-46659 | QLQLQESGPGLKKPSETLSLTCTVSGGSISSIPYYWGWIRKPPGKGLEWIGS | 36 |
| D111S-V2, CM1 | IYPSGSTDYNPSLKSRVTISVDTSKNQFSLKLSSVTAADTAVYYCARGGARD | |
| VH | RIDHAFSIWGQGTMVTVSS | |
| ADI-46659 | DIQMTQSPSSLSASVGDRVTITCQASQSISNYLNWYQEKPGKAPKLLIYDA | 37 |
| D111S-V2, CM1 | SNSETGVPSRFSGSGSGTDFTFTISSLQPEDIATYYCQQYYDLYKFGGGTKV | |
| VL | EIK | |
| ADI-46659 | QLQLQESGPGLKKPSETLSLTCTVSGGSISSIPYYWGWIRQPPGKGLEWIG | 38 |
| D111S-V2 (no | SIYPSGSTDYNPSLKSRVTISVDTSKNQFSLKLSSVTAADTAVYYCARGGAR | |
| CM) VH | DRIDHAFSIWGQGTMVTVSS | |
| ADI-46659 | DIQMTQSPSSLSASVGDRVTITCQASQSISNYLNWYQQKPGKAPKLLIYDA | 39 |
| D111S-V2 (no | SNSETGVPSRFSGSGSGTDFTFTISSLQPEDIATYYCQQYYDLYKFGGGTKV | |
| CM) VL | EIK | |
| ADI-46659 | QLQLQESGPGLVKPSETLSLTCTVSGGSISSIPYYWGWIRKPPGKGLEWIGS | 40 |
| D111S (no V2), | IYPSGSTDYNPSLKSRVTISVDTSKNQFSLKLSSVTAADTAVYYCARGGARD | |
| CM1 VH | RIDHAFSIWGQGTMVTVSS | |
| ADI-46659 | DIQMTQSPSSLSASVGDRVTITCQASQDISNYLNWYQEKPGKAPKLLIYDA | 41 |
| D111S (no V2), | SNLETGVPSRFSGSGSGTDFTFTISSLQPEDIATYYCQQYYDLYTFGGGTKV | |
| CM1 VL | EIK | |
| ADI-46659 | QLQLQESGPGLVKPSETLSLTCTVSGGSISSIPYYWGWIRQPPGKGLEWIG | 42 |
| D111S (no V2, no | SIYPSGSTDYNPSLKSRVTISVDTSKNQFSLKLSSVTAADTAVYYCARGGAR | |
| CM) VH | DRIDHAFSIWGQGTMVTVSS | |
| ADI-46659 | DIQMTQSPSSLSASVGDRVTITCQASQDISNYLNWYQQKPGKAPKLLIYD | 43 |
| D111S (no V2, no | ASNLETGVPSRFSGSGSGTDFTFTISSLQPEDIATYYCQQYYDLYTFGGGTK | |
| CM) VL | VEIK | |
| ADI-46659 | QLQLQESGPGLVKPSETLSLTCTVSGGSISSIPYYWGWIRQPPGKGLEWIG | 44 |
| D111E (no V2, | SIYPSGSTDYNPSLKSRVTISVDTSKNQFSLKLSSVTAADTAVYYCARGGAR | |
| no CM) VH | DRIDHAFEIWGQGTMVTVSS | |
| ADI-46659 (no | QLQLQESGPGLVKPSETLSLTCTVSGGSISSIPYYWGWIRQPP | 45 |
| D111S/E, no V2, | GKGLEWIGSIYPSGSTDYNPSLKSRVTISVDTSKNQFSLKLSS | |
| no CM) VH | VTAADTAVYYCARGGARDRIDHAFDIWGQGTMVTVSS | |
| ADI-46653 | QLQLQESGPGLKKPSETLSLTCTVSGGSISSESYYWGWIRKPPGKGLEWIG | 46 |
| D111E | SIYYSGSTDYNPSLKSRVTISVDTSKNQFSLKLSSVTAADTAVYYCARGGAS | |
| V2_H108K, CM1 | DRIDKAFEIWGQGTMVTVSS | |
| VH | | |
| ADI-46653 | DIQMTQSPSSLSASVGDRVTITCQASQSISNYLNWYQEKPGKAPKLLIYDA | 47 |
| D111E | SNSETGVPSRFSGSGSGTDFTFTISSLQPEDIATYYCQQYYDLYKFGGGTKV | |
| V2_H108K, CM1 | EIK | |
| VL | | |
| ADI-46653 | QLQLQESGPGLKKPSETLSLTCTVSGGSISSESYYWGWIRQPPGKGLEWIG | 48 |
| D111E | SIYYSGSTDYNPSLKSRVTISVDTSKNQFSLKLSSVTAADTAVYYCARGGAS | |
| V2 H108K (no | DRIDKAFEIWGQGTMVTVSS | |
| CM) VH | | |
| ADI-46653 | QLQLQESGPGLVKPSETLSLTCTVSGGSISSESYYWGWIRKPPGKGLEWIG | 49 |
| D111E H108K, | SIYYSGSTDYNPSLKSRVTISVDTSKNQFSLKLSSVTAADTAVYYCARGGAS | |
| CM1 VH | DRIDKAFEIWGQGTMVTVSS | |
| ADI-46653 | QLQLQESGPGLVKPSETLSLTCTVSGGSISSESYYWGWIRQPPGKGLEWIG | 50 |
| D111E H108K | SIYYSGSTDYNPSLKSRVTISVDTSKNQFSLKLSSVTAADTAVYYCARGGAS | |
| (no CM) VH | DRIDKAFEIWGQGTMVTVSS | |
| ADI-46653 | QLQLQESGPGLKKPSETLSLTCTVSGGSISSESYYWGWIRKPPGKGLEWIG | 51 |
| D111E_V2, CM1 | SIYYSGSTDYNPSLKSRVTISVDTSKNQFSLKLSSVTAADTAVYYCARGGAS | |
| VH | DRIDHAFEIWGQGTMVTVSS | |
| ADI-46653 | QLQLQESGPGLKKPSETLSLTCTVSGGSISSESYYWGWIRQPPGKGLEWIG | 52 |
| D111E_V2 (no | SIYYSGSTDYNPSLKSRVTISVDTSKNQFSLKLSSVTAADTAVYYCARGGAS | |
| CM) VH | DRIDHAFEIWGQGTMVTVSS | |
| ADI-46653 | QLQLQESGPGLVKPSETLSLTCTVSGGSISSESYYWGWIRKPPGKGLEWIG | 53 |
| D111E (CM1) | SIYYSGSTDYNPSLKSRVTISVDTSKNQFSLKLSSVTAADTAVYYCARGGAS | |
| VH | DRIDHAFEIWGQGTMVTVSS | |
| ADI-46653 | QLQLQESGPGLVKPSETLSLTCTVSGGSISSESYYWGWIRQPPGKGLEWIG | 54 |
| D111E (no CM) | SIYYSGSTDYNPSLKSRVTISVDTSKNQFSLKLSSVTAADTAVYYCARGGAS | |
| VH | DRIDHAFEIWGQGTMVTVSS | |
| ADI-46653 | QLQLQESGPGLVKPSETLSLTCTVSGGSISSESYYWGWIRQPPGKGLEWIG | 55 |
| D111S (no CM) | SIYYSGSTDYNPSLKSRVTISVDTSKNQFSLKLSSVTAADTAVYYCARGGAS | |
| VH | DRIDHAFSIWGQGTMVTVSS | |
| ADI-46653 (no | QLQLQESGPGLVKPSETLSLTCTVSGGSISSESYYWGWIRQPPGKGLEWIG | 56 |
| D111S/E, no V2, | SIYYSGSTDYNPSLKSRVTISVDTSKNQFSLSLSSVTAADTAVYYCARGGAS | |
| no CM) VH | DRIDHAFDIWGQGTMVTVSS | |

### Anti-IL-13 Binding Sites

Certain aspects of the present disclosure relate to binding proteins that comprise an antigen binding site that binds (e.g., specifically binds) an IL-13 polypeptide. In some embodiments, the IL-13 polypeptide is a human IL-13 polypeptide, also known as IL13 and P600. Human IL-13 polypeptides are known in the art and include, without limitation, the polypeptide represented by NCBI Accession Number NP_001341920.1, or a polypeptide produced from NCBI Gene ID Number 3596, which are incorporated herein by reference.

In some embodiments, a binding protein comprising an antigen binding site that binds an IL-13 polypeptide is monospecific and/or monovalent, bispecific and/or bivalent, trispecific and/or trivalent, or multispecific and/or multivalent.

In some embodiments, any of the CDRs and/or variable domains of the anti-IL-13 binding sites described below can be used in a monospecific antibody. In some embodiments, the monospecific antibody is a monoclonal antibody.

In some embodiments, any of the CDRs and/or variable domains of the anti-IL-13 binding sites described below can be used in a bispecific or multispecific antibody.

In some embodiments, any of the CDRs and/or variable domains of the anti-IL-13 binding sites described below (e.g., in Table 2) can be used in any binding site of a trispecific binding protein comprising four polypeptides that form three antigen binding sites, e.g., as described supra. In certain embodiments, a binding protein that comprises an antigen binding site that binds an IL-13 polypeptide is a trispecific binding protein comprising four polypeptides that form three antigen binding sites as described supra. In some embodiments, the V_{H2} and V_{L2} domains pair and form a second antigen binding site that binds an IL-13 polypeptide.

In some embodiments, a binding site that binds IL-13 comprises: a VH domain that comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59 or 108, and a VH domain that comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62.

In some embodiments, a binding site that binds IL-13 comprises: a VH domain that comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, and a VH domain that comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62.

In some embodiments, a binding site that binds IL-13 comprises: a VH domain that comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:108, and a VH domain that comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62.

In some embodiments, a binding site that binds IL-13 comprises: a VH domain that comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65 or 66, and a VH domain that comprises a CDR-L1 comprising the amino acid sequence of SEQ ID NO:67, a CDR-L2 comprising the amino acid sequence of KDS, and a CDR-L3 comprising the amino acid sequence of SEQ ID NO:69. In some embodiments, a binding site that binds IL-13 comprises: a VH domain that comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65, and a VH domain that comprises a CDR-L1 comprising the amino acid sequence of SEQ ID NO:67, a CDR-L2 comprising the amino acid sequence of KDS, and a CDR-L3 comprising the amino acid sequence of SEQ ID NO:69. In some embodiments, a binding site that binds IL-13 comprises: a VH domain that comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:66, and a VH domain that comprises a CDR-L1 comprising the amino acid sequence of SEQ ID NO:67, a CDR-L2 comprising the amino acid sequence of KDS, and a CDR-L3 comprising the amino acid sequence of SEQ ID NO:69.

In some embodiments, a binding site that binds IL-13 comprises: a VH domain that comprises an amino acid sequence that is at least 95%, at least 96%, at least 97%, at least 98%, at least 99%, or 100% identical to the amino acid sequence of SEQ ID NO:70 and/or a V_{L} domain that comprises an amino acid sequence that is at least 95%, at least 96%, at least 97%, at least 98%, at least 99%, or 100% identical to the amino acid sequence of SEQ ID NO:71. In some embodiments, a binding site that binds IL-13 comprises: a VH domain that comprises the amino acid sequence of SEQ ID NO:70 and/or a VH domain that comprises the amino acid sequence of SEQ ID NO:71. In some embodiments, a binding site that binds IL-13 comprises: a VH domain that comprises the amino acid sequence of SEQ ID NO:70 and a V_{L} domain that comprises the amino acid sequence of SEQ ID NO:71. In some embodiments, a binding site that binds IL-13 comprises: a VH domain that comprises 1, 2, or all 3 CDRs from a VH domain comprising the amino acid sequence of SEQ ID NO:70 and/or a VH domain that comprises 1, 2, or all 3 CDRs from a VH domain comprising the amino acid sequence of SEQ ID NO:71.

In some embodiments, a binding site that binds IL-13 comprises: a VH domain that comprises an amino acid sequence that is at least 95%, at least 96%, at least 97%, at least 98%, at least 99%, or 100% identical to the amino acid sequence of SEQ ID NO:72 and/or a V_{L} domain that comprises an amino acid sequence that is at least 95%, at least 96%, at least 97%, at least 98%, at least 99%, or 100% identical to the amino acid sequence of SEQ ID NO:73. In some embodiments, a binding site that binds IL-13 comprises: a VH domain that comprises the amino acid sequence of SEQ ID NO:72 and/or a VH domain that comprises the amino acid sequence of SEQ ID NO:73. In some embodiments, a binding site that binds IL-13 comprises: a VH domain that comprises the amino acid sequence of SEQ ID NO:72 and a V_{L} domain that comprises the amino acid sequence of SEQ ID NO:73. In some embodiments, a binding site that binds IL-13 comprises: a VH domain that comprises 1, 2, or all 3 CDRs from a VH domain comprising the amino acid sequence of SEQ ID NO:72 and/or a VH domain that comprises 1, 2, or all 3 CDRs from a VH domain comprising the amino acid sequence of SEQ ID NO:73.

In some embodiments, a binding site that binds IL-13 comprises: a VH domain that comprises an amino acid sequence that is at least 95%, at least 96%, at least 97%, at least 98%, at least 99%, or 100% identical to the amino acid sequence of SEQ ID NO:74 and/or a V_{L} domain that comprises an amino acid sequence that is at least 95%, at least 96%, at least 97%, at least 98%, at least 99%, or 100% identical to the amino acid sequence of SEQ ID NO:75. In some embodiments, a binding site that binds IL-13 comprises: a VH domain that comprises the amino acid sequence of SEQ ID NO:74 and/or a VH domain that comprises the amino acid sequence of SEQ ID NO:75. In some embodiments, a binding site that binds IL-13 comprises: a VH domain that comprises the amino acid sequence of SEQ ID NO:74 and a V_{L} domain that comprises the amino acid sequence of SEQ ID NO:75. In some embodiments, a binding site that binds IL-13 comprises: a VH domain that comprises 1, 2, or all 3 CDRs from a VH domain comprising the amino acid sequence of SEQ ID NO:74 and/or a VH domain that comprises 1, 2, or all 3 CDRs from a VH domain comprising the amino acid sequence of SEQ ID NO:75.

In some embodiments, a binding site that binds IL-13 comprises: a VH domain that comprises an amino acid sequence that is at least 95%, at least 96%, at least 97%, at least 98%, at least 99%, or 100% identical to the amino acid sequence of SEQ ID NO:76 and/or a V_{L} domain that comprises an amino acid sequence that is at least 95%, at least 96%, at least 97%, at least 98%, at least 99%, or 100% identical to the amino acid sequence of SEQ ID NO:77. In some embodiments, a binding site that binds IL-13 comprises: a VH domain that comprises the amino acid sequence of SEQ ID NO:76 and/or a VH domain that comprises the amino acid sequence of SEQ ID NO:77. In some embodiments, a binding site that binds IL-13 comprises: a VH domain that comprises the amino acid sequence of SEQ ID NO:76 and a V_{L} domain that comprises the amino acid sequence of SEQ ID NO:77. In some embodiments, a binding site that binds IL-13 comprises: a VH domain that comprises 1, 2, or all 3 CDRs from a VH domain comprising the amino acid sequence of SEQ ID NO:76 and/or a VH domain that comprises 1, 2, or all 3 CDRs from a VH domain comprising the amino acid sequence of SEQ ID NO:77.

Sequences of exemplary anti-IL-13 antigen binding sites are provided in Table 2. In some embodiments, a binding protein comprising an anti-IL-13 antigen binding site of the present disclosure comprises 1, 2, 3, 4, 5, or all 6 CDR sequences of an anti-IL-13 antibody described in Table 2. In some embodiments, a binding protein comprising an anti-IL-13 antigen binding site of the present disclosure comprises a VH domain sequence and/or VL domain sequence of an anti-IL-13 antibody described in Table 2.

TABLE 2
Anti-IL-13 binding protein sequences.

| Description | Sequence | SEQ ID NO |
|---|---|---|
| CL-203143_V3.4 | SYAMS | 57 |
| CDR-H1 (Kabat) | | |
| CL-203143_V3.4 | AISGSGGSTYYADSVKG | 58 |
| CDR-H2 (Kabat) | | |
| CL-203143_V3.4 | EYYYDPLQYYHYYYGMDV | 59 |
| CDR-H3 (Kabat) | | |
| CL-203143_no | EYYYDPLNTYHYYYGMDV | 108 |
| V3.4 CDR-H3 | | |
| (Kabat) | | |
| CL-203143_V3.4 | SGDALPKQYAY | 60 |
| CDR-L1 (Kabat) | | |
| CL-203143_V3.4 | KDSERPS | 61 |
| CDR-L2 (Kabat) | | |
| CL-203143_V3.4 | QSADSSGTYWV | 62 |
| CDR-L3 (Kabat) | | |
| CL-203143_V3.4 | GFTFSSYA | 63 |
| CDR-H1 (IMGT) | | |
| CL-203143_V3.4 | ISGSGGST | 64 |
| CDR-H2 (IMGT) | | |
| CL-203143_V3.4 | AREYYYDPLQYYHYYYGMDV | 65 |
| CDR-H3 (IMGT) | | |
| CL-203143_no | AREYYYDPLNTYHYYYGMDV | 66 |
| V3.4 CDR-H3 | | |
| (IMGT) | | |
| CL-203143_V3.4 | ALPKQY | 67 |
| CDR-L1 (IMGT) | | |
| CL-203143_V3.4 | KDS | 68 |
| CDR-L2 (IMGT) | | |
| CL-203143_V3.4 | QSADSSGTYWV | 69 |
| CDR-L3 (IMGT) | | |
| CL-203143_V3.4 | EVQLLESGGGLVQPGGSLRLSCAASGFTFSSYAMSWVREAPGKGLEWVS | 70 |
| CM2 VH | AISGSGGSTYYADSVKGRFTISRDNSKNTLYLQMNSLRAEDTAVYYCAREY | |
| | YYDPLQYYHYYYGMDVWGQGTTVTVSS | |
| CL-203143_V3.4 | SYELTQPPSVSVSPGQTARITCSGDALPKQYAYWYQKKPGQAPVLVIYKDS | 71 |
| CM2 VL | ERPSGIPERFSGSSKGTTVTLTISGVQAEDEADYYCQSADSSGTYWVFGGG | |
| | TKLTVL | |
| CL-203143_V3.4 | EVQLLESGGGLVQPGGSLRLSCAASGFTFSSYAMSWVRKAPGKGLEWVS | 72 |
| CM1 VH | AISGSGGSTYYADSVKGRFTISRDNSKNTLYLQMNSLRAEDTAVYYCAREY | |
| | YYDPLQYYHYYYGMDVWGQGTTVTVSS | |
| CL-203143_V3.4 | SYELTQPPSVSVSPGQTARITCSGDALPKQYAYWYQEKPGQAPVLVIYKDS | 73 |
| CM1 VL | ERPSGIPERFSGSSKGTTVTLTISGVQAEDEADYYCQSADSSGTYWVFGGG | |
| | TKLTVL | |
| CL-203143_V3.4 | EVQLLESGGGLVQPGGSLRLSCAASGFTFSSYAMSWVRQAPGKGLEWVS | 74 |
| (no CM) VH | AISGSGGSTYYADSVKGRFTISRDNSKNTLYLQMNSLRAEDTAVYYCAREY | |
| | YYDPLQYYHYYYGMDVWGQGTTVTVSS | |
| CL-203143_V3.4 | SYELTQPPSVSVSPGQTARITCSGDALPKQYAYWYQQKPGQAPVLVIYKD | 75 |
| (no CM) VL | SERPSGIPERFSGSSKGTTVTLTISGVQAEDEADYYCQSADSSGTYWVFGG | |
| | GTKLTVL | |
| CL-203143 (no | EVQLLESGGGLVQPGGSLRLSCAASGFTFSSYAMSWVRQAPGKGLEWVS | 76 |
| V3.4, no CM) VH | AISGSGGSTYYADSVKGRFTISRDNSKNTLYLQMNSLRAEDTAVYYCAREY | |
| | YYDPLNTYHYYYGMDVWGQGTTVTVSS | |
| CL-203143 (no | SYELMQPPSVSVSPGQTARITCSGDALPKQYAYWYQQKPGQAPVLVIYKD | 77 |
| V3.4, no CM) VL | SERPSGIPERFSGSSSGTTVTLTISGVQAEDEADYYCQSADSSGTYWVFGG | |
| | GTKLTVL | |

### Anti-OX40L Binding Sites

Certain aspects of the present disclosure relate to binding proteins that comprise an antigen binding site that binds (e.g., specifically binds) an OX40L polypeptide. In some embodiments, the OX40L polypeptide is a human OX40L polypeptide, also known as tumor necrosis factor (TNF) superfamily member 4 (TNFSF4), GP34, CD252, TXGP1, CD134L, OX-40L, and TNLG2B. Human OX40L polypeptides are known in the art and include, without limitation, the polypeptide represented by NCBI Accession Number NP_001284491.1, or a polypeptide produced from NCBI Gene ID Number 7292. In some embodiments, the OX40L polypeptide comprises the amino acid sequence

(SEQ ID NO: 110)
MERVQPLEENVGNAARPRFERNKLLLVASVIQGLGLLLCFTYICLHFSA
LQVSHRYPRIQSIKVQFTEYKKEKGFILTSQKEDEIMKVQNNSVIINCD
GFYLISLKGYFSQEVNISLHYQKDEEPLFQLKKVRSVNSLMVASLTYKD
KVYLNVTTDNTSLDDFHVNGGELILIHQNPGEFCVL.

In some embodiments, a binding protein comprising an antigen binding site that binds an OX40L polypeptide is monospecific and/or monovalent, bispecific and/or bivalent, trispecific and/or trivalent, or multispecific and/or multivalent.

In some embodiments, any of the CDRs and/or variable domains of the anti-OX40L binding sites described below can be used in a monospecific antibody. In some embodiments, the monospecific antibody is a monoclonal antibody.

In some embodiments, any of the CDRs and/or variable domains of the anti-OX40L binding sites described below can be used in a bispecific or multispecific antibody.

In some embodiments, any of the CDRs and/or variable domains of the anti-OX40L binding sites described below (e.g., in Table 3) can be used in any binding site of a trispecific binding protein comprising four polypeptides that form three antigen binding sites, e.g., as described supra. In certain embodiments, a binding protein that comprises an antigen binding site that binds an OX40L polypeptide is a trispecific binding protein comprising four polypeptides that form three antigen binding sites as described supra. In some embodiments, the V_{H3} and V_{L3} domains pair and form a third antigen binding site that binds an OX40L polypeptide.

In some embodiments, the binding site that specifically binds an OX40L polypeptide binds to an epitope of human OX40L that at least partially overlaps with that of human OX40. In some embodiments, the binding site that specifically binds an OX40L polypeptide binds to an epitope of human OX40L comprising one or more of the following residues (numbering based on SEQ ID NO:110): Q65, T67, E68, T78, Q80, K106, G107, Y108, F109, S110, Q111, E112, D122, E123, E124, F127, Q128, L129, K130, K131, R133, S134, N136, D156, N157, T158, S159, L160, D161, D162, H164, and N166. In some embodiments, the binding site that specifically binds an OX40L polypeptide binds to an epitope of human OX40L comprising all of the following residues (numbering based on SEQ ID NO:110): Q65, T67, E68, T78, Q80, K106, G107, Y108, F109, 5110, Q111, E112, D122, E123, E124, F127, Q128, L129, K130, K131, R133, 5134, N136, D156, N157, T158, 5159, L160, D161, D162, H164, and N166. In some embodiments, residues Q65, T67, E68, T78, Q80, K106, G107, Y108, F109, S110, Q111, E112, K131, R133, 5134, N136 D156, N157, T158, 5159, L160, D161, D162, H164, and N166 are part of a first OX40L monomer, and residues D122, E123, E124, F127, Q128, L129, and K130 are part of a second OX40L monomer.

In some embodiments, the binding site that specifically binds an OX40L polypeptide binds to human OX40L with a paratope comprising one or more of the following VH domain residues: F27, T28, N30/530, 531, Y32, D50, 552, 554, R56, 557, K98, D99, F102, C103, 5104, A106, 5107, C108, N109, E110, and Y113 (consecutive numbering of SEQ ID NO:97) and/or one or more of the following V_{L} domain residues: R30, N31, D32, S56, H91, S92, R93, Y94, P95, and W96 (consecutive numbering of SEQ ID NO:96). In some embodiments, the binding site that specifically binds an OX40L polypeptide binds to human OX40L with a paratope comprising one or more of the following VH domain residues: F27, T28, N30/S30, S31, Y32, D50, S52, S54, R56, S57, K98, D99, F102, C103, S104, A106, S107, C108, N109, E110, and Y113 (consecutive numbering of SEQ ID NO:97) and one or more of the following V_{L} domain residues: R30, N31, D32, S56, H91, S92, R93, Y94, P95, and W96 (consecutive numbering of SEQ ID NO:96). In some embodiments, the binding site that specifically binds an OX40L polypeptide binds to human OX40L with a paratope comprising all of the following VH domain residues: F27, T28, N30/S30, S31, Y32, D50, S52, S54, R56, S57, K98, D99, F102, C103, S104, A106, S107, C108, N109, E110, and Y113 (consecutive numbering of SEQ ID NO:97) and all of the following V_{L} domain residues: R30, N31, D32, S56, H91, S92, R93, Y94, P95, and W96 (consecutive numbering of SEQ ID NO:96).

In some embodiments, a binding site that binds OX40L comprises: a VH domain that comprises a CDR-H1 comprising the amino acid sequence of SEQ ID NO:78, a CDR-H2 comprising the amino acid sequence of SEQ ID NO:79, and a CDR-H3 comprising the amino acid sequence of SEQ ID NO:80, and V_{L} domain that comprises a CDR-L1 comprising the amino acid sequence of SEQ ID NO:81, a CDR-L2 comprising the amino acid sequence of SEQ ID NO:82, and a CDR-L3 comprising the amino acid sequence of SEQ ID NO:83.

In some embodiments, a binding site that binds OX40L comprises: a VH domain that comprises a CDR-H1 comprising the amino acid sequence of SEQ ID NO:84 or 85, a CDR-H2 comprising the amino acid sequence of SEQ ID NO:86, and a CDR-H3 comprising the amino acid sequence of SEQ ID NO:87, and V_{L} domain that comprises a CDR-L1 comprising the amino acid sequence of SEQ ID NO:88, a CDR-L2 comprising the amino acid sequence of SEQ ID NO:89, and a CDR-L3 comprising the amino acid sequence of SEQ ID NO:90. In some embodiments, a binding site that binds OX40L comprises: a VH domain that comprises a CDR-H1 comprising the amino acid sequence of SEQ ID NO:84, a CDR-H2 comprising the amino acid sequence of SEQ ID NO:86, and a CDR-H3 comprising the amino acid sequence of SEQ ID NO:87, and V_{L} domain that comprises a CDR-L1 comprising the amino acid sequence of SEQ ID NO:88, a CDR-L2 comprising the amino acid sequence of SEQ ID NO:89, and a CDR-L3 comprising the amino acid sequence of SEQ ID NO:90. In some embodiments, a binding site that binds OX40L comprises: a VH domain that comprises a CDR-H1 comprising the amino acid sequence of SEQ ID NO:85, a CDR-H2 comprising the amino acid sequence of SEQ ID NO:86, and a CDR-H3 comprising the amino acid sequence of SEQ ID NO:87, and V_{L} domain that comprises a CDR-L1 comprising the amino acid sequence of SEQ ID NO:88, a CDR-L2 comprising the amino acid sequence of SEQ ID NO:89, and a CDR-L3 comprising the amino acid sequence of SEQ ID NO:90.

In some embodiments, a binding site that binds OX40L comprises: a VH domain that comprises an amino acid sequence that is at least 95%, at least 96%, at least 97%, at least 98%, at least 99%, or 100% identical to the amino acid sequence of SEQ ID NO:91 and/or a V_{L} domain that comprises an amino acid sequence that is at least 95%, at least 96%, at least 97%, at least 98%, at least 99%, or 100% identical to the amino acid sequence of SEQ ID NO:92. In some embodiments, a binding site that binds OX40L comprises: a VH domain that comprises the amino acid sequence of SEQ ID NO:91 and/or a VH domain that comprises the amino acid sequence of SEQ ID NO:92. In some embodiments, a binding site that binds OX40L comprises: a VH domain that comprises the amino acid sequence of SEQ ID NO:91 and a VH domain that comprises the amino acid sequence of SEQ ID NO:92. In some embodiments, a binding site that binds OX40L comprises: a VH domain that comprises 1, 2, or all 3 CDRs from a VH domain comprising the amino acid sequence of SEQ ID NO:91 and/or a VH domain that comprises 1, 2, or all 3 CDRs from a VH domain comprising the amino acid sequence of SEQ ID NO:92.

In some embodiments, a binding site that binds OX40L comprises: a VH domain that comprises an amino acid sequence that is at least 95%, at least 96%, at least 97%, at least 98%, at least 99%, or 100% identical to the amino acid sequence of SEQ ID NO:93 and/or a V_{L} domain that comprises an amino acid sequence that is at least 95%, at least 96%, at least 97%, at least 98%, at least 99%, or 100% identical to the amino acid sequence of SEQ ID NO:94. In some embodiments, a binding site that binds OX40L comprises: a VH domain that comprises the amino acid sequence of SEQ ID NO:93 and/or a VH domain that comprises the amino acid sequence of SEQ ID NO:94. In some embodiments, a binding site that binds OX40L comprises: a VH domain that comprises the amino acid sequence of SEQ ID NO:93 and a VH domain that comprises the amino acid sequence of SEQ ID NO:94. In some embodiments, a binding site that binds OX40L comprises: a VH domain that comprises 1, 2, or all 3 CDRs from a VH domain comprising the amino acid sequence of SEQ ID NO:93 and/or a VH domain that comprises 1, 2, or all 3 CDRs from a VH domain comprising the amino acid sequence of SEQ ID NO:94.

In some embodiments, a binding site that binds OX40L comprises: a VH domain that comprises an amino acid sequence that is at least 95%, at least 96%, at least 97%, at least 98%, at least 99%, or 100% identical to the amino acid sequence of SEQ ID NO:95 and/or a V_{L} domain that comprises an amino acid sequence that is at least 95%, at least 96%, at least 97%, at least 98%, at least 99%, or 100% identical to the amino acid sequence of SEQ ID NO:96. In some embodiments, a binding site that binds OX40L comprises: a VH domain that comprises the amino acid sequence of SEQ ID NO:95 and/or a VH domain that comprises the amino acid sequence of SEQ ID NO:96. In some embodiments, a binding site that binds OX40L comprises: a VH domain that comprises the amino acid sequence of SEQ ID NO:95 and a VH domain that comprises the amino acid sequence of SEQ ID NO:96. In some embodiments, a binding site that binds OX40L comprises: a VH domain that comprises 1, 2, or all 3 CDRs from a VH domain comprising the amino acid sequence of SEQ ID NO:95 and/or a VH domain that comprises 1, 2, or all 3 CDRs from a VH domain comprising the amino acid sequence of SEQ ID NO:96.

In some embodiments, a binding site that binds OX40L comprises: a VH domain that comprises an amino acid sequence that is at least 95%, at least 96%, at least 97%, at least 98%, at least 99%, or 100% identical to the amino acid sequence of SEQ ID NO:97 and/or a V_{L} domain that comprises an amino acid sequence that is at least 95%, at least 96%, at least 97%, at least 98%, at least 99%, or 100% identical to the amino acid sequence of SEQ ID NO:96. In some embodiments, a binding site that binds OX40L comprises: a VH domain that comprises the amino acid sequence of SEQ ID NO:97 and/or a VH domain that comprises the amino acid sequence of SEQ ID NO:96. In some embodiments, a binding site that binds OX40L comprises: a VH domain that comprises the amino acid sequence of SEQ ID NO:97 and a VH domain that comprises the amino acid sequence of SEQ ID NO:96. In some embodiments, a binding site that binds OX40L comprises: a VH domain that comprises 1, 2, or all 3 CDRs from a VH domain comprising the amino acid sequence of SEQ ID NO: 97 and/or a VH domain that comprises 1, 2, or all 3 CDRs from a VH domain comprising the amino acid sequence ofSEQ ID NO:96.

Sequences of exemplary anti-OX40L antigen binding sites are provided in Table 3. In some embodiments, a binding protein comprising an anti-OX40L antigen binding site of the present disclosure comprises 1, 2, 3, 4, 5, or all 6 CDR sequences of an anti-OX40L antibody described in Table 3. In some embodiments, a binding protein comprising an anti-OX40L antigen binding site of the present disclosure comprises a VH domain sequence and/or V_{L} domain sequence of an anti-OX40L antibody described in Table 3.

TABLE 3
Anti-OX40L binding protein sequences.

| Description | Sequence | SEQ ID NO |
|---|---|---|
| MM25PL3A2_V | SYAMT | 78 |
| 1CDR-H1 | | |
| (Kabat) | | |
| MM25PL3A2_V | DISGSGRSIFYADSVKG | 79 |
| 1CDR-H2 | | |
| (Kabat) | | |
| MM25PL3A2_V | DLGFCSGASCNEADY | 80 |
| 1CDR-H3 | | |
| (Kabat) | | |
| MM25PL3A2_V | RASQGIRNDLD | 81 |
| 1CDR-L1 | | |
| (Kabat) | | |
| MM25PL3A2 V | ATSGLQS | 82 |
| 1CDR-L2 | | |
| (Kabat) | | |
| MM25PL3A2_V | LQHSRYPWT | 83 |
| 1CDR-L3 | | |
| (Kabat) | | |
| MM25PL3A2_V | GFTFSSYA | 84 |
| 1CDR-H1 | | |
| (IMGT) | | |
| MM25PL3A2 | GFTFNSYA | 85 |
| (no VI, no CM) | | |
| CDR-H1 IMGT | | |
| MM25PL3A2_V | ISGSGRSI | 86 |
| 1CDR-H2 | | |
| (IMGT) | | |
| MM25PL3A2_V | AKDLGFCSGASCNEADY | 87 |
| 1CDR-H3 | | |
| (IMGT) | | |
| MM25PL3A2_V | QGIRND | 88 |
| 1CDR-L1 | | |
| (IMGT) | | |
| MM25PL3A2_V | ATS | 89 |
| 1CDR-L2 | | |
| (IMGT) | | |
| MM25PL3A2_V | LQHSRYPWT | 90 |
| 1CDR-L3 | | |
| (IMGT) | | |
| MM25PL3A2_V | EVQLVESGGGFVQPGMSLRLSCAASGFTFSSYAMTWVRKAPGKGLEWV | 91 |
| 1 CM1 VH | SDISGSGRSIFYADSVKGRFTISRDNSKRTLYLQMNSLRAEDTAIYYCAKDL | |
| | GFCSGASCNEADYWGQGTLVTVSS | |
| MM25PL3A2_V | DIQMTQSPSSLSASVGDRVTITCRASQGIRNDLDWYQEKPGKAPKRLIYAT | 92 |
| 1 CM1 VL | SGLQSGVPSRFSGSGSGTEFTLTISSLQPEDFATYYCLQHSRYPWTFGQGT | |
| | KVEIK | |
| MM25PL3A2_V | EVQLVESGGGFVQPGMSLRLSCAASGFTFSSYAMTWVREAPGKGLEWV | 93 |
| 1, CM2 VH | SDISGSGRSIFYADSVKGRFTISRDNSKRTLYLQMNSLRAEDTAIYYCAKDL | |
| | GFCSGASCNEADYWGQGTLVTVSS | |
| MM25PL3A2_V | DIQMTQSPSSLSASVGDRVTITCRASQGIRNDLDWYQKKPGKAPKRLIYAT | 94 |
| 1, CM2 VL | SGLQSGVPSRFSGSGSGTEFTLTISSLQPEDFATYYCLQHSRYPWTFGQGT | |
| | KVEIK | |
| MM25PL3A2_V | EVQLVESGGGFVQPGMSLRLSCAASGFTFSSYAMTWVRHAPGKGLEWV | 95 |
| 1 (no CM) VH | SDISGSGRSIFYADSVKGRFTISRDNSKRTLYLQMNSLRAEDTAIYYCAKDL | |
| | GFCSGASCNEADYWGQGTLVTVSS | |
| MM25PL3A2_V | DIQMTQSPSSLSASVGDRVTITCRASQGIRNDLDWYQQKPGKAPKRLIYA | 96 |
| 1 (no CM) VL | TSGLQSGVPSRFSGSGSGTEFTLTISSLQPEDFATYYCLQHSRYPWTFGQG | |
| | TKVEIK | |
| MM25PL3A2 | EVQLVESGGGFVQPGMSLRLSCAASGFTFNSYAMTWVRHAPGKGLEWV | 97 |
| (no VI, no CM) | SDISGSGRSIFYADSVKGRFTISRDNSKRTLYLQMNSLRAEDTAIYYCAKDL | |
| VH | GFCSGASCNEADYWGQGTLVTVSS | |

### Anti-IL-17 Binding Sites

Certain aspects of the present disclosure relate to binding proteins that comprise an antigen binding site that binds (e.g., specifically binds) an IL-17 polypeptide. In some embodiments, the IL-17 polypeptide is a human IL-17 polypeptide. Exemplary human IL-17 polypeptides include IL-17A and IL-17F. IL-17A is also known as IL17, IL17A, CTLA8, IL-17, ILA17, and CTLA-8. Human IL-17A polypeptides are known in the art and include, without limitation, the polypeptide represented by NCBI Accession Number NP_002181.1, or a polypeptide produced from NCBI Gene ID Number 3605. IL-17F is also known as ML1, ML-1, IL17F, and CANDF6. Human IL-17F polypeptides are known in the art and include, without limitation, the polypeptide represented by NCBI Accession Number NP_443104.1, or a polypeptide produced from NCBI Gene ID Number 112744. In some embodiments, an antigen binding site that binds an IL-17 polypeptide binds an IL-17A polypeptide. In some embodiments, an antigen binding site that binds an IL-17 polypeptide binds an IL-17A homodimer. In some embodiments, an antigen binding site that binds an IL-17 polypeptide binds an IL-17F polypeptide. In some embodiments, an antigen binding site that binds an IL-17 polypeptide binds an IL-17F homodimer. In some embodiments, an antigen binding site that binds an IL-17 polypeptide binds an IL-17A/F heterodimer.

In some embodiments, a binding protein comprising an antigen binding site that binds an IL-17 polypeptide is monospecific and/or monovalent, bispecific and/or bivalent, trispecific and/or trivalent, or multispecific and/or multivalent.

In some embodiments, any of the CDRs and/or variable domains of the anti-IL-17 binding sites described below can be used in a monospecific antibody. In some embodiments, the monospecific antibody is a monoclonal antibody.

In some embodiments, any of the CDRs and/or variable domains of the anti-IL-17 binding sites described below can be used in a bispecific or multispecific antibody.

In some embodiments, any of the CDRs and/or variable domains of the anti-IL-17 binding sites described below (e.g., in Table 48) can be used in any binding site of a trispecific binding protein comprising four polypeptides that form three antigen binding sites, e.g., as described supra. In certain embodiments, a binding protein that comprises an antigen binding site that binds an IL-17 polypeptide is a trispecific binding protein comprising four polypeptides that form three antigen binding sites as described supra. In some embodiments, the V_{H3} and V_{L3} domains pair and form a third antigen binding site that binds an IL-17 polypeptide.

In some embodiments, a binding site that binds IL-17 binds (e.g., specifically binds, is capable of binding, is capable of specifically binding) IL-17A (e.g., human IL-17A, such as that having the amino acid sequence of SEQ ID NO:156). In some embodiments, a binding site that binds IL-17 binds (e.g., specifically binds, is capable of binding, is capable of specifically binding) IL-17A (e.g., human IL-17A, such as that having the amino acid sequence of SEQ ID NO:156) and IL-17AF (e.g., human IL-17AF). In some embodiments, a binding site that binds IL-17 binds (e.g., specifically binds, is capable of binding, is capable of specifically binding) IL-17A (e.g., human IL-17A, such as that having the amino acid sequence of SEQ ID NO:156), IL-17AF (e.g., human IL-17AF), and IL-17F (e.g., human IL-17F). In some embodiments, the binding site that specifically binds an IL-17 polypeptide binds to an epitope that spans both chains (i.e., Chains A and B) of a homodimer of IL-17A, wherein the epitope comprises one or more of the following residues in Chain A (numbering based on SEQ ID NO:156): L66, N70, D72, N74, D76, Y77, H78, M79, N80, V82, V116, T117, P118, 1119, V120, H121, H122, V123, and A124, and one or more of the following residues in Chain B (numbering based on SEQ ID NO:156): R31, Y35, R38, N70, A71, and D72.

In some embodiments, the binding site that specifically binds an IL-17 polypeptide binds to an epitope that spans both chains (i.e., Chains A and B) of a homodimer of IL-17A, wherein the epitope comprises each of the following residues in Chain A (numbering based on SEQ ID NO:122): L66, N70, D72, N74, D76, Y77, H78, M79, N80, V82, V116, T117, P118, I119, V120, H121, H122, V123, and A124, and each of the following residues in Chain B (numbering based on SEQ ID NO:156): R31, Y35, R38, N70, A71, and D72.

In some embodiments, the binding site that specifically binds an IL-17 polypeptide binds to an epitope that spans both chains (i.e., Chains A and B) of a homodimer of IL-17A, wherein the epitope comprises one or more of the following residues in Chain A (numbering based on SEQ ID NO:122): L66, N70, A71, D72, N74, D76, Y77, H78, M79, N80, V82, V116, T117, P118, 1119, V120, H121, H122, V123, and A124, and one or more of the following residues in Chain B (numbering based on SEQ ID NO:156): N70, A71, and D72.

In some embodiments, the binding site that specifically binds an IL-17 polypeptide binds to an epitope that spans both chains (i.e., containing Chains A and B) of a homodimer of IL-17A, wherein the epitope comprises each of the following residues in Chain A (numbering based on SEQ ID NO:156): L66, N70, A71, D72, N74, D76, Y77, H78, M79, N80, V82, V116, T117, P118, 1119, V120, H121, H122, V123, and A124, and each of the following residues in Chain B (numbering based on SEQ ID NO:156): N70, A71, and D72.

In some embodiments, a single homodimer of IL-17A (i.e., Chains A and B) may be bound by two separate IL-17 binding sites (i.e., Binding Sites A and B), wherein Binding Site A specifically binds to an epitope that spans Chains A and B of the IL-17A homodimer, wherein the epitope comprises one or more of the following residues in Chain A (numbering based on SEQ ID NO:156): L66, N70, D72, N74, D76, Y77, H78, M79, N80, V82, V116, T117, P118, 1119, V120, H121, H122, V123, and A124, and one or more of the following residues in Chain B (numbering based on SEQ ID NO:156): R31, Y35, R38, N70, A71, and D72; and wherein Binding Site B specifically binds to an epitope that spans Chains A and B of the IL-17A homodimer, wherein the epitope comprises one or more of the following residues in Chain B (numbering based on SEQ ID NO:156): L66, N70, A71, D72, N74, D76, Y77, H78, M79, N80, V82, V116, T117, P118, 1119, V120, H121, H122, V123, and A124, and one or more of the following residues in Chain A (numbering based on SEQ ID NO:156): N70, A71, and D72.

In some embodiments, a single homodimer of IL-17A (i.e., Chains A and B) may be bound by two separate IL-17 binding sites (i.e., Binding Sites A and B), wherein Binding Site A specifically binds to an epitope that spans Chains A and B of the IL-17A homodimer, wherein the epitope comprises each of the following residues in Chain A (numbering based on SEQ ID NO:122): L66, N70, D72, N74, D76, Y77, H78, M79, N80, V82, V116, T117, P118, 1119, V120, H121, H122, V123, and A124, and each of the following residues in Chain B (numbering based on SEQ ID NO:156): R31, Y35, R38, N70, A71, and D72; and wherein Binding Site B specifically binds to an epitope that spans Chains A and B of the IL-17A homodimer, wherein the epitope comprises each of the following residues in Chain B (numbering based on SEQ ID NO:156): L66, N70, A71, D72, N74, D76, Y77, H78, M79, N80, V82, V116, T117, P118, 1119, V120, H121, H122, V123, and A124, and each of the following residues in Chain A (numbering based on SEQ ID NO:156): N70, A71, and D72.

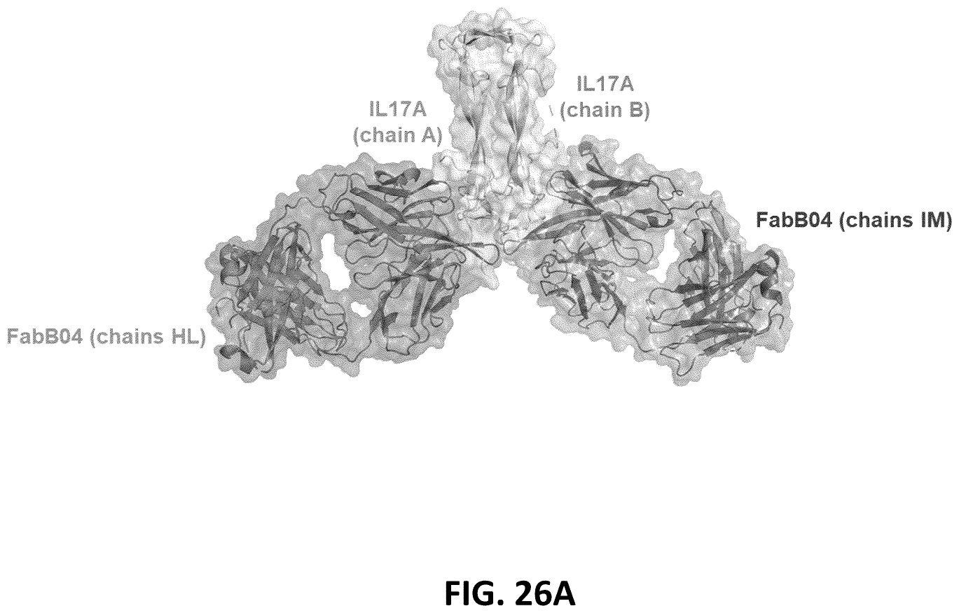

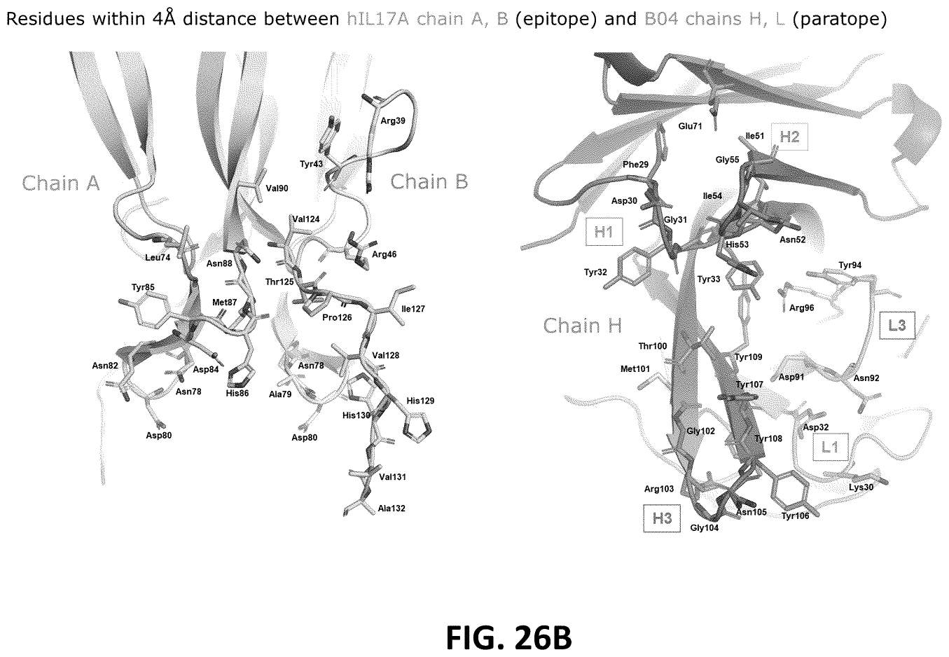

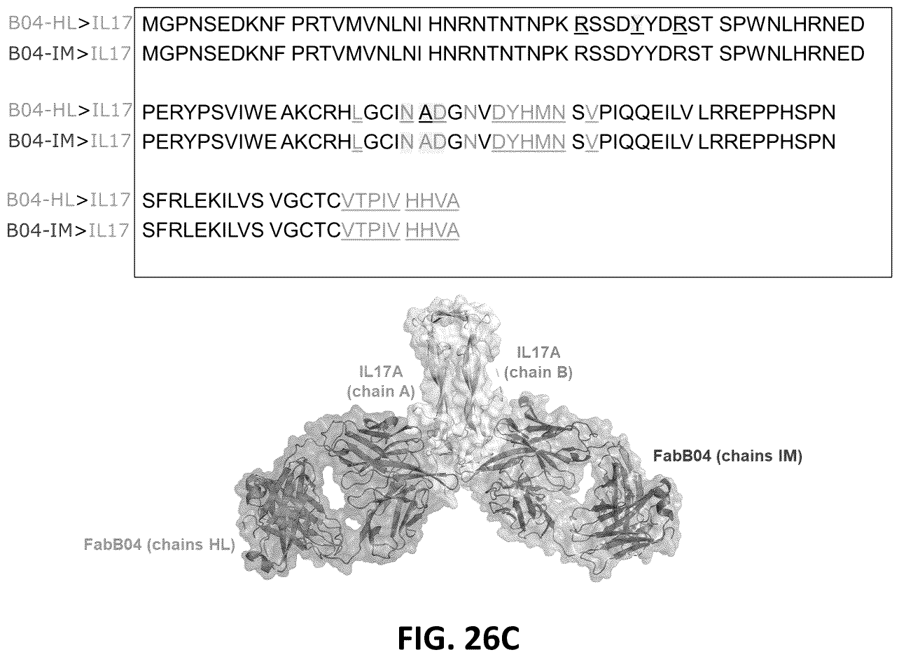

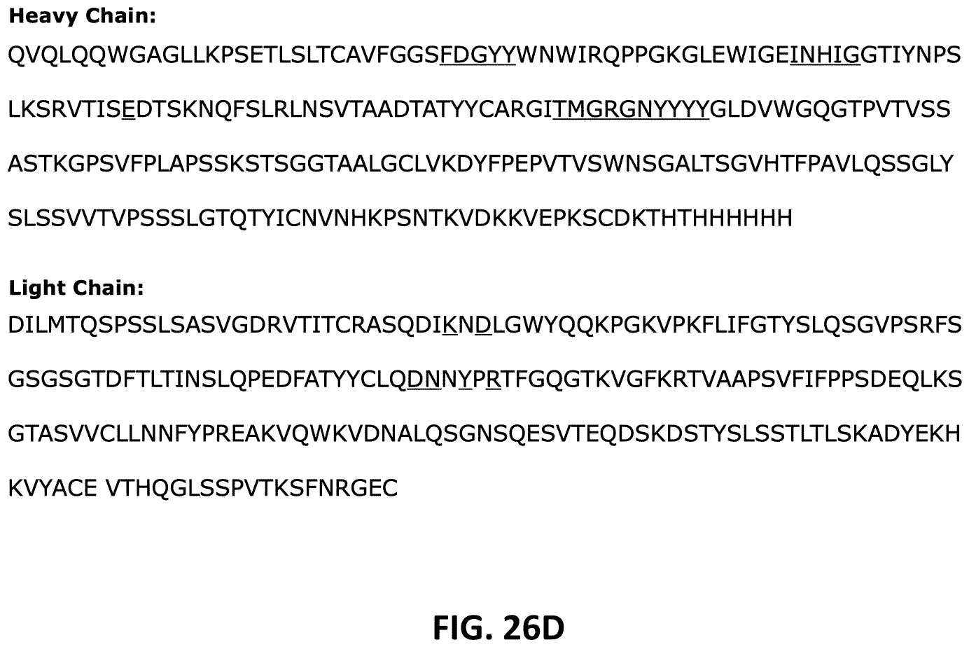

In some embodiments, the binding site that specifically binds an IL-17 polypeptide binds to human IL-17A and has a paratope comprising one or more of the following VH domain residues: F29, D30, G31, Y32, Y33, 151, N52, H53, 154, G55, E71, T100, M101, G102, R103, G104, N105, Y106, Y107, Y108, and Y109 (consecutive numbering of SEQ ID NO:154 as shown in FIG. 26D, which can be converted to other numbering schemes like Kabat or IMGT). In some embodiments, the binding site that specifically binds an IL-17 polypeptide binds to human IL-17A and has a paratope comprising one or more of the following V_{L} domain residues: K30, D32, D91, N92, Y94, and R96 (consecutive numbering of SEQ ID NO:155 as shown in FIG. 26D, which can be converted to other numbering schemes like Kabat or IMGT). In some embodiments, the binding site that specifically binds an IL-17 polypeptide binds to human IL-17A and has a paratope comprising one or more of the following VH domain residues: F29, D30, G31, Y32, Y33, 151, N52, H53, 154, G55, E71, T100, M101, G102, R103, G104, N105, Y106, Y107, Y108, and Y109 (consecutive numbering of SEQ ID NO:154 as shown in FIG. 26D, which can be converted to other numbering schemes like Kabat or IMGT), and one or more of the following V_{L} domain residues: K30, D32, D91, N92, Y94, and R96 (consecutive numbering of SEQ ID NO:155 as shown in FIG. 26D, which can be converted to other numbering schemes like Kabat or IMGT). In some embodiments, the binding site that specifically binds an IL-17 polypeptide binds to human IL-17A and has a paratope comprising each of the following VH domain residues: F29, D30, G31, Y32, Y33, I51, N52, H53, I54, G55, E71, T100, M101, G102, R103, G104, N105, Y106, Y107, Y108, and Y109 (consecutive numbering of SEQ ID NO:154 as shown in FIG. 26D, which can be converted to other numbering schemes like Kabat or IMGT). In some embodiments, the binding site that specifically binds an IL-17 polypeptide binds to human IL-17A and has a paratope comprising each of the following V_{L} domain residues: K30, D32, D91, N92, Y94, and R96 (consecutive numbering of SEQ ID NO: 155 as shown in FIG. 26D, which can be converted to other numbering schemes like Kabat or IMGT). In some embodiments, the binding site that specifically binds an IL-17 polypeptide binds to human IL-17A and has a paratope comprising each of the following VH domain residues: F29, D30, G31, Y32, Y33, 151, N52, H53, 154, G55, E71, T100, M101, G102, R103, G104, N105, Y106, Y107, Y108, and Y109 (consecutive numbering of SEQ ID NO: 154 as shown in FIG. 26D, which can be converted to other numbering schemes like Kabat or IMGT), and each of the following V_{L} domain residues: K30, D32, D91, N92, Y94, and R96 (consecutive numbering of SEQ ID NO:155 as shown in FIG. 26D, which can be converted to other numbering schemes like Kabat or IMGT).

In some embodiments, a binding site that binds IL-17 comprises: a VH domain that comprises a CDR-H1 comprising the amino acid sequence of SEQ ID NO:117, a CDR-H2 comprising the amino acid sequence of SEQ ID NO:118, and a CDR-H3 comprising the amino acid sequence of SEQ ID NO:119, and V_{L} domain that comprises a CDR-L1 comprising the amino acid sequence of SEQ ID NO:114, a CDR-L2 comprising the amino acid sequence of TFF, and a CDR-L3 comprising the amino acid sequence of SEQ ID NO:116.

In some embodiments, a binding site that binds IL-17 comprises: a VH domain that comprises a CDR-H1 comprising the amino acid sequence of SEQ ID NO:123, a CDR-H2 comprising the amino acid sequence of SEQ ID NO:124, and a CDR-H3 comprising the amino acid sequence of SEQ ID NO:125, and V_{L} domain that comprises a CDR-L1 comprising the amino acid sequence of SEQ ID NO:120, a CDR-L2 comprising the amino acid sequence of GTY, and a CDR-L3 comprising the amino acid sequence of SEQ ID NO:122.

In some embodiments, a binding site that binds IL-17 comprises: a VH domain that comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131, and V_{L} domain that comprises a CDR-L1 comprising the amino acid sequence of SEQ ID NO:126, a CDR-L2 comprising the amino acid sequence of GTY, and a CDR-L3 comprising the amino acid sequence of SEQ ID NO:128.

In some embodiments, a binding site that binds IL-17 comprises: a VH domain that comprises an amino acid sequence that is at least 95%, at least 96%, at least 97%, at least 98%, at least 99%, or 100% identical to the amino acid sequence of SEQ ID NO:132 and/or a V_{L} domain that comprises an amino acid sequence that is at least 95%, at least 96%, at least 97%, at least 98%, at least 99%, or 100% identical to the amino acid sequence of SEQ ID NO: 133. In some embodiments, a binding site that binds IL-17 comprises: a VH domain that comprises the amino acid sequence of SEQ ID NO: 132 and/or a VH domain that comprises the amino acid sequence of SEQ ID NO:133. In some embodiments, a binding site that binds IL-17 comprises: a VH domain that comprises the amino acid sequence of SEQ ID NO: 132 and a VH domain that comprises the amino acid sequence of SEQ ID NO:133. In some embodiments, a binding site that binds IL-17 comprises: a VH domain that comprises 1, 2, or all 3 CDRs from a VH domain comprising the amino acid sequence of SEQ ID NO:132 and/or a VH domain that comprises 1, 2, or all 3 CDRs from a VH domain comprising the amino acid sequence of SEQ ID NO:133.

In some embodiments, a binding site that binds IL-17 comprises: a VH domain that comprises an amino acid sequence that is at least 95%, at least 96%, at least 97%, at least 98%, at least 99%, or 100% identical to the amino acid sequence of SEQ ID NO:134 and/or a V_{L} domain that comprises an amino acid sequence that is at least 95%, at least 96%, at least 97%, at least 98%, at least 99%, or 100% identical to the amino acid sequence of SEQ ID NO: 135. In some embodiments, a binding site that binds IL-17 comprises: a VH domain that comprises the amino acid sequence of SEQ ID NO: 134 and/or a VH domain that comprises the amino acid sequence of SEQ ID NO:135. In some embodiments, a binding site that binds IL-17 comprises: a VH domain that comprises the amino acid sequence of SEQ ID NO: 134 and a VH domain that comprises the amino acid sequence of SEQ ID NO:135. In some embodiments, a binding site that binds IL-17 comprises: a VH domain that comprises 1, 2, or all 3 CDRs from a VH domain comprising the amino acid sequence of SEQ ID NO:134 and/or a VH domain that comprises 1, 2, or all 3 CDRs from a VH domain comprising the amino acid sequence of SEQ ID NO:135.

In some embodiments, a binding site that binds IL-17 comprises: a VH domain that comprises an amino acid sequence that is at least 95%, at least 96%, at least 97%, at least 98%, at least 99%, or 100% identical to the amino acid sequence of SEQ ID NO:136 and/or a V_{L} domain that comprises an amino acid sequence that is at least 95%, at least 96%, at least 97%, at least 98%, at least 99%, or 100% identical to the amino acid sequence of SEQ ID NO: 137. In some embodiments, a binding site that binds IL-17 comprises: a VH domain that comprises the amino acid sequence of SEQ ID NO: 136 and/or a VH domain that comprises the amino acid sequence of SEQ ID NO:137. In some embodiments, a binding site that binds IL-17 comprises: a VH domain that comprises the amino acid sequence of SEQ ID NO: 136 and a VH domain that comprises the amino acid sequence of SEQ ID NO: 137. In some embodiments, a binding site that binds IL-17 comprises: a VH domain that comprises 1, 2, or all 3 CDRs from a VH domain comprising the amino acid sequence of SEQ ID NO: 136 and/or a VH domain that comprises 1, 2, or all 3 CDRs from a VH domain comprising the amino acid sequence of SEQ ID NO:137.

Sequences of exemplary anti-IL-17 antigen binding sites are provided in Table 3. In some embodiments, a binding protein comprising an anti-IL-17 antigen binding site of the present disclosure comprises 1, 2, 3, 4, 5, or all 6 CDR sequences of an anti-IL-17 antibody described in Table 48. In some embodiments, a binding protein comprising an anti-IL-17 antigen binding site of the present disclosure comprises a VH domain sequence and/or VL domain sequence of an anti-IL-17 antibody described in Table 48.

TABLE 48
Anti-IL-17 binding protein sequences.

| Description | Sequence | SEQ ID NO |
|---|---|---|
| MMB05PL10 | QSLLDSDDGNTY | 114 |
| D10 v01-CM2 | | |
| CDR-L1 (IMGT) | | |
| MMB05PL10 | TFF | N/A |
| D10 v01-CM2 | | |
| CDR-L2 (IMGT) | | |
| MMB05PL10 | MQRIEFPYT | 116 |
| D10 v01-CM2 | | |
| CDR-L3 (IMGT) | | |
| MMB05PL10 | GFTFSDYF | 117 |
| D10 v01-CM2 | | |
| CDR-H1 (IMGT) | | |
| MMB05PL10 | ISSTGTTK | 118 |
| D10 v01-CM2 | | |
| CDR-H2 (IMGT) | | |
| MMB05PL10 | ARDGSSLDS | 119 |
| D10 v01-CM2 | | |
| CDR-H3 (IMGT) | | |
| CL-495369 | QDIKND | 120 |
| VH_N52F_VL03 | | |
| -CM2 CDR-L1 | | |
| (IMGT) | | |
| CL-495369 | GTY | N/A |
| VH N52F VL03 | | |
| -CM2 CDR-L2 | | |
| (IMGT) | | |
| CL-495369 | LQDNNYPRT | 122 |
| VH N52F VL03 | | |
| -CM2 CDR-L3 | | |
| (IMGT) | | |
| CL-495369 | GGSFDGYY | 123 |
| VH N52F_VL03 | | |
| -CM2 CDR-H1 | | |
| (IMGT) | | |
| CL-495369 | IFHIGGT | 124 |
| VH N52F VL03 | | |
| -CM2 CDR-H2 | | |
| (IMGT) | | |
| CL-495369 | ARGITMGRGNYYYYGLDV | 125 |
| VH_N52F_VL03 | | |
| -CM2 CDR-H3 | | |
| (IMGT) | | |
| IL-17 CL- | QDIKND | 126 |
| 495369 VH-wt- | | |
| v1.1 VL-v3- | | |
| CM2 CDR-L1 | | |
| (IMGT) | | |
| IL-17_CL- | GTY | N/A |
| 495369 VH-wt- | | |
| v1.1_VL-v3- | | |
| CM2 CDR-L2 | | |
| (IMGT) | | |
| IL-17 CL- | LQDNNYPRT | 128 |
| 495369 VH-wt- | | |
| v1.1 VL-v3- | | |
| CM2 CDR-L3 | | |
| (IMGT) | | |
| IL-17 CL- | GGSFDGYY | 129 |
| 495369 VH-wt- | | |
| v1.1 VL-v3- | | |
| CM2 CDR-H1 | | |
| (IMGT) | | |
| IL-17 CL- | INHIGGT | 130 |
| 495369 VH-wt- | | |
| v1.1 VL-v3- | | |
| CM2 CDR-H2 | | |
| (IMGT) | | |
| IL-17_CL- | ARGITMGRGNYYYYGLDV | 131 |
| 495369 VH-wt- | | |
| v1.1 VL-v3- | | |
| CM2 CDR-H3 | | |
| (IMGT) | | |
| MMB05PL10 | EVQLVESGGGLVKPGGSLRLSCAASGFTFSDYFMSWIREAPGKGLEWV | 132 |
| D10 v01-CM2 | SYISSTGTTKYYADSVKGRFTMSRDNAKNSLYLQMSSLRAEDTAVYYCA | |
| VH | RDGSSLDSWGQGTLVTVSS | |
| MMB05PL10 | DIVMTQSPLSLPVTPGEPASISCRSSQSLLDSDDGNTYLDWFLKKPGQSP | 133 |
| D10 v01-CM2 | QLLIYTFFYRASGVPDRFSGSGSGTDFTLKISRVEAEDVGVYYCMQRIEF | |
| VL | PYTFGQGTKLEIK | |
| CL-495369 | QVQLQQWGAGLLKPSETLSLTCAVFGGSFDGYYWNWIREPPGKGLEWI | 134 |
| VH_N52F_VL03 | GEIFHIGGTIYNPSLKSRVTISEDTSKNQFSLRLNSVTAADTATYYCARGIT | |
| -CM2 VH | MGRGNYYYYGLDVWGQGTPVTVSS | |
| CL-495369 | DIQMTQSPSSLSASVGDRVTITCRASQDIKNDLGWYQKKPGKAPKFLIFG | 135 |
| VH N52F VL03 | TYSLQSGVPSRFSGSGSGTEFTLTISSLQPEDFATYYCLQDNNYPRTFG | |
| -CM2 VL | QGTKVEIK | |
| IL-17 CL- | QVQLQQWGAGLLKPSETLSLTCAVYGGSFDGYYWNWIREPPGKGLEWI | 136 |
| 495369 VH-wt- | GEINHIGGTIYNPSLKSRVTISEDTSKNQFSLKLSSVTAADTAVYYCARGIT | |
| | MGRGNYYYYGLDVWGQGTTVTVSS | |
| v1.1 VL-v3- | | |
| CM2 VH | | |
| IL-17 CL- | DIQMTQSPSSLSASVGDRVTITCRASQDIKNDLGWYQKKPGKAPKFLIFG | 137 |
| 495369 VH-wt- | TYSLQSGVPSRFSGSGSGTEFTLTISSLQPEDFATYYCLQDNNYPRTFG | |
| v1.1 VL-v3- | QGTKVEIK | |
| CM2 VL | | |

### Anti-NKG2D Binding Sites

Certain aspects of the present disclosure relate to binding proteins that comprise an antigen binding site that binds (e.g., specifically binds) an NKG2D polypeptide. In some embodiments, the NKG2D polypeptide is a human NKG2D polypeptide. Human NKG2D polypeptides are known in the art and include, without limitation, the polypeptide represented by NCBI Accession Number NP_031386.2, or a polypeptide produced from NCBI Gene ID Number 22914. NKG2D is also known as KLR, CD314, KLRK1, NKG2-D, and D12S2489E. In some embodiments, a human NKG2D polypeptide comprises the amino acid sequence of

(SEQ ID NO: 254)
MGWIRGRRSRHSWEMSEFHNYNLDLKKSDFSTRWQKQRCPVVKSKCREN
ASPFFFCCFIAVAMGIRFIIMVTIWSAVFLNSLFNQEVQIPLTESYCGP
CPKNWICYKNNCYQFFDESKNWYESQASCMSQNASLLKVYSKEDQDLLK
LVKSYHWMGLVHIPTNGSWQWEDGSILSPNLLTIIEMQKGDCALYASSF
KGYIENCSTPNTYICMQRTV

In some embodiments, a binding protein comprising an antigen binding site that binds an NKG2D polypeptide is monospecific and/or monovalent, bispecific and/or bivalent, trispecific and/or trivalent, or multispecific and/or multivalent.

In some embodiments, any of the CDRs and/or variable domains of the anti-NKG2D binding sites described below can be used in a monospecific antibody. In some embodiments, the monospecific antibody is a monoclonal antibody.

In some embodiments, any of the CDRs and/or variable domains of the anti-NKG2D binding sites described below can be used in a bispecific or multispecific antibody.

In some embodiments, any of the CDRs and/or variable domains of the anti-NKG2D binding sites described below (e.g., in Table 49) can be used in any binding site of a trispecific binding protein comprising four polypeptides that form three antigen binding sites, e.g., as described supra. In certain embodiments, a binding protein that comprises an antigen binding site that binds an NKG2D polypeptide is a trispecific binding protein comprising four polypeptides that form three antigen binding sites as described supra. In some embodiments, the V_{H3} and V_{L3} domains pair and form a third antigen binding site that binds an NKG2D polypeptide.

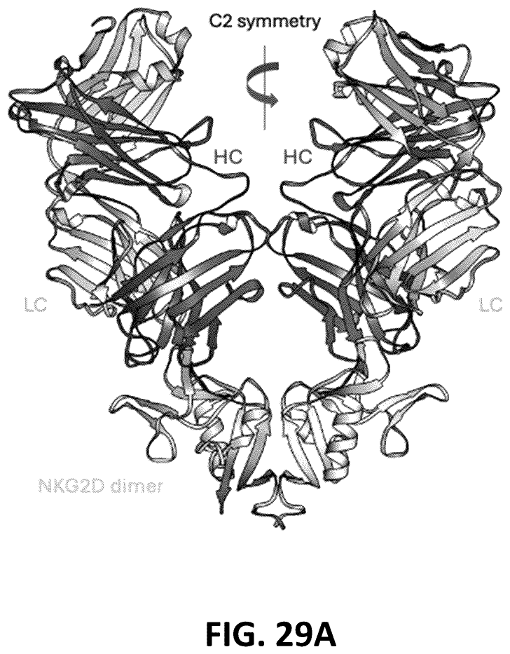

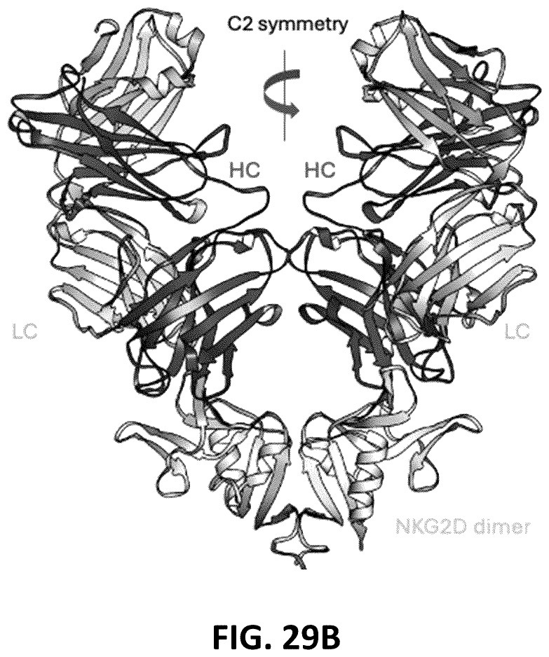

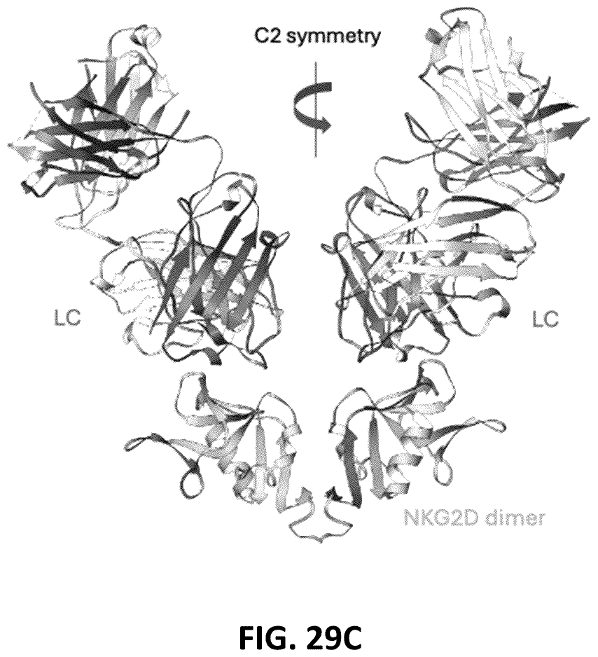

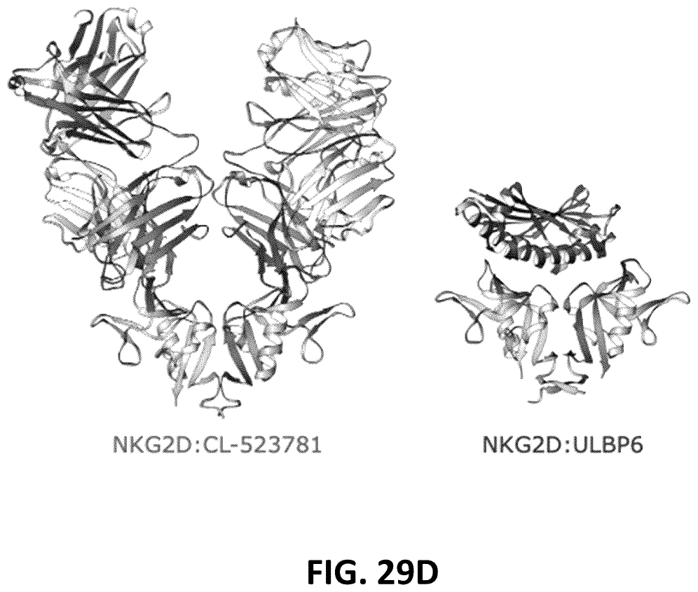

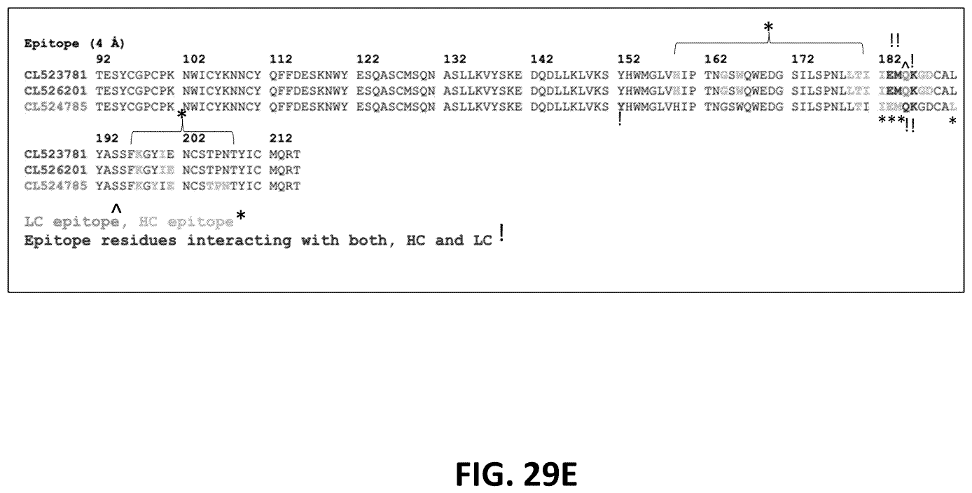

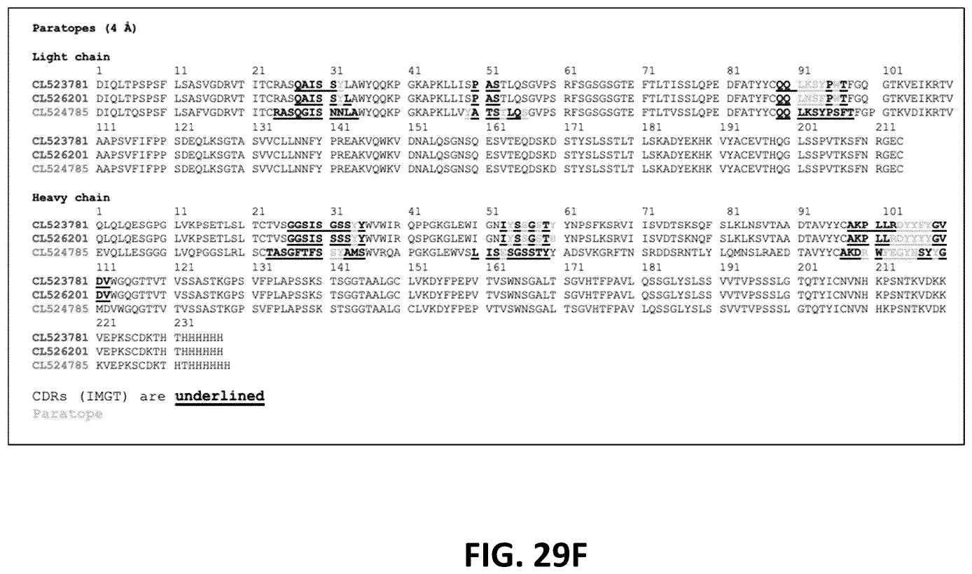

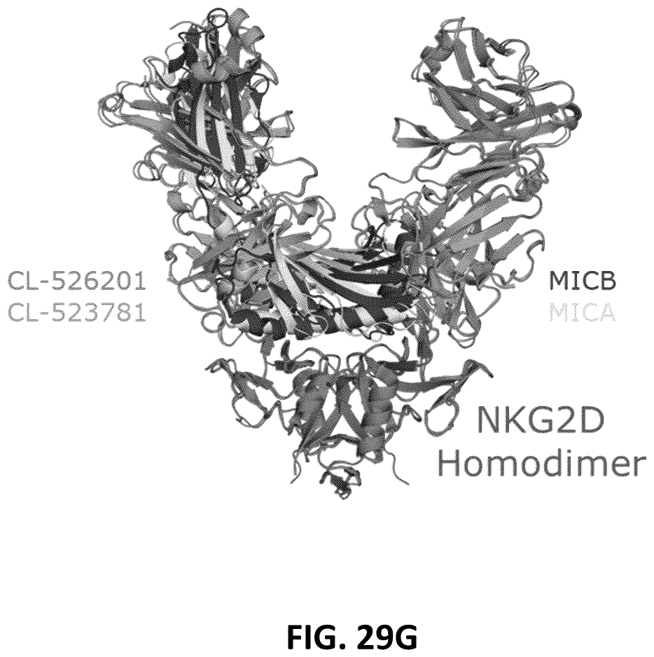

In some embodiments, binding of the binding site that specifically binds an NKG2D polypeptide to the NKG2D polypeptide disrupts the NGK2D dimer. In some embodiments, binding of the binding site that specifically binds an NKG2D polypeptide to the NKG2D polypeptide blocks binding between NKG2D and MICA/MICB. In some embodiments, binding of the binding site that specifically binds an NKG2D polypeptide to the NKG2D polypeptide blocks binding between NKG2D and ULBP6. In some embodiments, the binding site that specifically binds an NKG2D polypeptide binds to an epitope of human NKG2D that at least partially overlaps with a binding site for MICA, MICB, and/or ULBP6. In some embodiments, the binding site that specifically binds an NKG2D polypeptide binds to an epitope of human NKG2D comprising one or more of the following residues (numbering based on SEQ ID NO:254): H159, G164, W166, L179, T180, 1181, 1182, E183, M184, Q185, K186, G187, D188, K197, and 1200. In some embodiments, the binding site that specifically binds an NKG2D polypeptide binds to an epitope of human NKG2D comprising each of the following residues (numbering based on SEQ ID NO:254): H159, G164, W166, L179, T180, 1181, 1182, E183, M184, Q185, K186, G187, D188, K197, and 1200. In some embodiments, the binding site that specifically binds an NKG2D polypeptide binds to an epitope of human NKG2D comprising one or more of the following residues (numbering based on SEQ ID NO:254): H159, G164, W166, L179, T180, 1181, 1182, E183, M184, Q185, K186, G187, D188, K197, 1200, and E201. In some embodiments, the binding site that specifically binds an NKG2D polypeptide binds to an epitope of human NKG2D comprising each of the following residues (numbering based on SEQ ID NO:254): H159, G164, W166, L179, T180, 1181, 1182, E183, M184, Q185, K186, G187, D188, K197, 1200, and E201. In some embodiments, the binding site that specifically binds an NKG2D polypeptide binds to an epitope of human NKG2D comprising one or more of the following residues (numbering based on SEQ ID NO:254): Y152, T180, 1182, E183, M184, Q185, K186, L191, K197, Y199, E201, T205, P206, and N207. In some embodiments, the binding site that specifically binds an NKG2D polypeptide binds to an epitope of human NKG2D comprising each of the following residues (numbering based on SEQ ID NO:254): Y152, T180, I182, E183, M184, Q185, K186, L191, K197, Y199, E201, T205, P206, and N207. In some embodiments, the binding site that specifically binds an NKG2D polypeptide binds to human NKG2D with a paratope comprising one or more of the following VH domain residues: Y34, Y54, S56, S58, Y60, D104, Y105, Y106, F107, and Y108 (consecutive numbering of SEQ ID NO:227 as shown in FIG. 29F, which can be converted to other numbering schemes like Kabat or IMGT). In some embodiments, the binding site that specifically binds an NKG2D polypeptide binds to human NKG2D with a paratope comprising one or more of the following V_{L} domain residues: Y32, L91, K92, S93, Y94, and W96 (consecutive numbering of SEQ ID NO:228 as shown in FIG. 29F, which can be converted to other numbering schemes like Kabat or IMGT). In some embodiments, the binding site that specifically binds an NKG2D polypeptide binds to human NKG2D with a paratope comprising each of the following VH domain residues: Y34, Y54, S56, S58, Y60, D104, Y105, Y106, F107, and Y108 (consecutive numbering of SEQ ID NO:227 as shown in FIG. 29F, which can be converted to other numbering schemes like Kabat or IMGT). In some embodiments, the binding site that specifically binds an NKG2D polypeptide binds to human NKG2D with a paratope comprising each of the following V_{L} domain residues: Y32, L91, K92, S93, Y94, and W96 (consecutive numbering of SEQ ID NO:228 as shown in FIG. 29F, which can be converted to other numbering schemes like Kabat or IMGT). In some embodiments, the binding site that specifically binds an NKG2D polypeptide binds to human NKG2D with a paratope comprising one or more of the following VH domain residues: Y34, Y54, S56, S58, Y60, R103, D104, Y105, Y106, F107, and Y108 (consecutive numbering of SEQ ID NO:231 as shown in FIG. 29F, which can be converted to other numbering schemes like Kabat or IMGT). In some embodiments, the binding site that specifically binds an NKG2D polypeptide binds to human NKG2D with a paratope comprising one or more of the following V_{L} domain residues: Y32, L91, K92, S93, Y94, and W96 (consecutive numbering of SEQ ID NO:232 as shown in FIG. 29F, which can be converted to other numbering schemes like Kabat or IMGT). In some embodiments, the binding site that specifically binds an NKG2D polypeptide binds to human NKG2D with a paratope comprising each of the following VH domain residues: Y34, Y54, S56, S58, Y60, R103, D104, Y105, Y106, F107, and Y108 (consecutive numbering of SEQ ID NO:231 as shown in FIG. 29F, which can be converted to other numbering schemes like Kabat or IMGT). In some embodiments, the binding site that specifically binds an NKG2D polypeptide binds to human NKG2D with a paratope comprising each of the following V_{L} domain residues: Y32, L91, K92, S93, Y94, and W96 (consecutive numbering of SEQ ID NO:232 as shown in FIG. 29F, which can be converted to other numbering schemes like Kabat or IMGT). In some embodiments, the binding site that specifically binds an NKG2D polypeptide binds to human NKG2D with a paratope comprising one or more of the following VH domain residues: S31, Y32, E53, R100, F102, E103, G104, Y105, H106, and Y109 (consecutive numbering of SEQ ID NO:229 as shown in FIG. 29F, which can be converted to other numbering schemes like Kabat or IMGT). In some embodiments, the binding site that specifically binds an NKG2D polypeptide binds to human NKG2D with a paratope comprising one or more of the following V_{L} domain residues: Y49, T53, and S56 (consecutive numbering of SEQ ID NO:230 as shown in FIG. 29F, which can be converted to other numbering schemes like Kabat or IMGT). In some embodiments, the binding site that specifically binds an NKG2D polypeptide binds to human NKG2D with a paratope comprising each of the following V_{H} domain residues: S31, Y32, E53, R100, F102, E103, G104, Y105, H106, and Y109 (consecutive numbering of SEQ ID NO:229 as shown in FIG. 29F, which can be converted to other numbering schemes like Kabat or IMGT). In some embodiments, the binding site that specifically binds an NKG2D polypeptide binds to human NKG2D with a paratope comprising each of the following V_{L} domain residues: Y49, T53, and S56 (consecutive numbering of SEQ ID NO:230 as shown in FIG. 29F, which can be converted to other numbering schemes like Kabat or IMGT)

In some embodiments, a binding site that binds NKG2D comprises: a VH domain that comprises a CDR-H1 comprising the amino acid sequence of SEQ ID NO:157, a CDR-H2 comprising the amino acid sequence of SEQ ID NO:158, and a CDR-H3 comprising the amino acid sequence of SEQ ID NO:159, and a VH domain that comprises a CDR-L1 comprising the amino acid sequence of SEQ ID NO:160, a CDR-L2 comprising the amino acid sequence of RDS, and a CDR-L3 comprising the amino acid sequence of SEQ ID NO:162.

In some embodiments, a binding site that binds NKG2D comprises: a VH domain that comprises a CDR-H1 comprising the amino acid sequence of SEQ ID NO:163, a CDR-H2 comprising the amino acid sequence of SEQ ID NO:164, and a CDR-H3 comprising the amino acid sequence of SEQ ID NO:165, and a VH domain that comprises a CDR-L1 comprising the amino acid sequence of SEQ ID NO:166, a CDR-L2 comprising the amino acid sequence of YDD, and a CDR-L3 comprising the amino acid sequence of SEQ ID NO:168.

In some embodiments, a binding site that binds NKG2D comprises: a VH domain that comprises a CDR-H1 comprising the amino acid sequence of SEQ ID NO:169, a CDR-H2 comprising the amino acid sequence of SEQ ID NO:170, and a CDR-H3 comprising the amino acid sequence of SEQ ID NO:171, and a VH domain that comprises a CDR-L1 comprising the amino acid sequence of SEQ ID NO:172, a CDR-L2 comprising the amino acid sequence of YDD, and a CDR-L3 comprising the amino acid sequence of SEQ ID NO:174.

In some embodiments, a binding site that binds NKG2D comprises: a VH domain that comprises a CDR-H1 comprising the amino acid sequence of SEQ ID NO:175, a CDR-H2 comprising the amino acid sequence of SEQ ID NO:176, and a CDR-H3 comprising the amino acid sequence of SEQ ID NO:177, and a VH domain that comprises a CDR-L1 comprising the amino acid sequence of SEQ ID NO:178, a CDR-L2 comprising the amino acid sequence of YDD, and a CDR-L3 comprising the amino acid sequence of SEQ ID NO:180.

In some embodiments, a binding site that binds NKG2D comprises: a VH domain that comprises a CDR-H1 comprising the amino acid sequence of SEQ ID NO:181, a CDR-H2 comprising the amino acid sequence of SEQ ID NO:182, and a CDR-H3 comprising the amino acid sequence of SEQ ID NO:183, and a VH domain that comprises a CDR-L1 comprising the amino acid sequence of SEQ ID NO:184, a CDR-L2 comprising the amino acid sequence of QDT, and a CDR-L3 comprising the amino acid sequence of SEQ ID NO:186.

In some embodiments, a binding site that binds NKG2D comprises: a VH domain that comprises a CDR-H1 comprising the amino acid sequence of SEQ ID NO:187, a CDR-H2 comprising the amino acid sequence of SEQ ID NO:188, and a CDR-H3 comprising the amino acid sequence of SEQ ID NO:189, and a VH domain that comprises a CDR-L1 comprising the amino acid sequence of SEQ ID NO:190, a CDR-L2 comprising the amino acid sequence of PAS, and a CDR-L3 comprising the amino acid sequence of SEQ ID NO:192.

In some embodiments, a binding site that binds NKG2D comprises: a VH domain that comprises a CDR-H1 comprising the amino acid sequence of SEQ ID NO:193, a CDR-H2 comprising the amino acid sequence of SEQ ID NO:194, and a CDR-H3 comprising the amino acid sequence of SEQ ID NO:195, and a VH domain that comprises a CDR-L1 comprising the amino acid sequence of SEQ ID NO:196, a CDR-L2 comprising the amino acid sequence of ATS, and a CDR-L3 comprising the amino acid sequence of SEQ ID NO:198.

In some embodiments, a binding site that binds NKG2D comprises: a VH domain that comprises a CDR-H1 comprising the amino acid sequence of SEQ ID NO:199, a CDR-H2 comprising the amino acid sequence of SEQ ID NO:200, and a CDR-H3 comprising the amino acid sequence of SEQ ID NO:201, and a VH domain that comprises a CDR-L1 comprising the amino acid sequence of SEQ ID NO:202, a CDR-L2 comprising the amino acid sequence of PAS, and a CDR-L3 comprising the amino acid sequence of SEQ ID NO:204.

In some embodiments, a binding site that binds NKG2D comprises: a VH domain that comprises a CDR-H1 comprising the amino acid sequence of SEQ ID NO:205, a CDR-H2 comprising the amino acid sequence of SEQ ID NO:206, and a CDR-H3 comprising the amino acid sequence of SEQ ID NO:207, and a VH domain that comprises a CDR-L1 comprising the amino acid sequence of SEQ ID NO:208, a CDR-L2 comprising the amino acid sequence of AAS, and a CDR-L3 comprising the amino acid sequence of SEQ ID NO:210.

In some embodiments, a binding site that binds NKG2D comprises: a VH domain that comprises a CDR-H1 comprising the amino acid sequence of SEQ ID NO:211, a CDR-H2 comprising the amino acid sequence of SEQ ID NO:212, and a CDR-H3 comprising the amino acid sequence of SEQ ID NO:213, and a VH domain that comprises a CDR-L1 comprising the amino acid sequence of SEQ ID NO:214, a CDR-L2 comprising the amino acid sequence of YAS, and a CDR-L3 comprising the amino acid sequence of SEQ ID NO:216.

In some embodiments, a binding site that binds NKG2D comprises: a VH domain that comprises an amino acid sequence that is at least 95%, at least 96%, at least 97%, at least 98%, at least 99%, or 100% identical to the amino acid sequence of SEQ ID NO:217 and/or a V_{L} domain that comprises an amino acid sequence that is at least 95%, at least 96%, at least 97%, at least 98%, at least 99%, or 100% identical to the amino acid sequence of SEQ ID NO:218. In some embodiments, a binding site that binds NKG2D comprises: a VH domain that comprises the amino acid sequence of SEQ ID NO:217 and/or a VH domain that comprises the amino acid sequence of SEQ ID NO:218. In some embodiments, a binding site that binds NKG2D comprises: a VH domain that comprises the amino acid sequence of SEQ ID NO:217 and a VH domain that comprises the amino acid sequence of SEQ ID NO:218. In some embodiments, a binding site that binds NKG2D comprises: a VH domain that comprises 1, 2, or all 3 CDRs from a VH domain comprising the amino acid sequence of SEQ ID NO:217 and/or a VH domain that comprises 1, 2, or all 3 CDRs from a VH domain comprising the amino acid sequence of SEQ ID NO:218.

In some embodiments, a binding site that binds NKG2D comprises: a VH domain that comprises an amino acid sequence that is at least 95%, at least 96%, at least 97%, at least 98%, at least 99%, or 100% identical to the amino acid sequence of SEQ ID NO:219 and/or a V_{L} domain that comprises an amino acid sequence that is at least 95%, at least 96%, at least 97%, at least 98%, at least 99%, or 100% identical to the amino acid sequence of SEQ ID NO:220. In some embodiments, a binding site that binds NKG2D comprises: a VH domain that comprises the amino acid sequence of SEQ ID NO:219 and/or a VH domain that comprises the amino acid sequence of SEQ ID NO:220. In some embodiments, a binding site that binds NKG2D comprises: a VH domain that comprises the amino acid sequence of SEQ ID NO:219 and a VH domain that comprises the amino acid sequence of SEQ ID NO:220. In some embodiments, a binding site that binds NKG2D comprises: a VH domain that comprises 1, 2, or all 3 CDRs from a VH domain comprising the amino acid sequence of SEQ ID NO:219 and/or a VH domain that comprises 1, 2, or all 3 CDRs from a VH domain comprising the amino acid sequence of SEQ ID NO:220.

In some embodiments, a binding site that binds NKG2D comprises: a VH domain that comprises an amino acid sequence that is at least 95%, at least 96%, at least 97%, at least 98%, at least 99%, or 100% identical to the amino acid sequence of SEQ ID NO:221 and/or a V_{L} domain that comprises an amino acid sequence that is at least 95%, at least 96%, at least 97%, at least 98%, at least 99%, or 100% identical to the amino acid sequence of SEQ ID NO:222. In some embodiments, a binding site that binds NKG2D comprises: a VH domain that comprises the amino acid sequence of SEQ ID NO:221 and/or a VH domain that comprises the amino acid sequence of SEQ ID NO:222. In some embodiments, a binding site that binds NKG2D comprises: a VH domain that comprises the amino acid sequence of SEQ ID NO:221 and a VH domain that comprises the amino acid sequence of SEQ ID NO:222. In some embodiments, a binding site that binds NKG2D comprises: a VH domain that comprises 1, 2, or all 3 CDRs from a VH domain comprising the amino acid sequence of SEQ ID NO:221 and/or a VH domain that comprises 1, 2, or all 3 CDRs from a VH domain comprising the amino acid sequence of SEQ ID NO:222.

In some embodiments, a binding site that binds NKG2D comprises: a VH domain that comprises an amino acid sequence that is at least 95%, at least 96%, at least 97%, at least 98%, at least 99%, or 100% identical to the amino acid sequence of SEQ ID NO:223 and/or a V_{L} domain that comprises an amino acid sequence that is at least 95%, at least 96%, at least 97%, at least 98%, at least 99%, or 100% identical to the amino acid sequence of SEQ ID NO:224. In some embodiments, a binding site that binds NKG2D comprises: a VH domain that comprises the amino acid sequence of SEQ ID NO:223 and/or a VH domain that comprises the amino acid sequence of SEQ ID NO:224. In some embodiments, a binding site that binds NKG2D comprises: a VH domain that comprises the amino acid sequence of SEQ ID NO:223 and a VH domain that comprises the amino acid sequence of SEQ ID NO:224. In some embodiments, a binding site that binds NKG2D comprises: a VH domain that comprises 1, 2, or all 3 CDRs from a VH domain comprising the amino acid sequence of SEQ ID NO:223 and/or a VH domain that comprises 1, 2, or all 3 CDRs from a VH domain comprising the amino acid sequence of SEQ ID NO:224.

In some embodiments, a binding site that binds NKG2D comprises: a VH domain that comprises an amino acid sequence that is at least 95%, at least 96%, at least 97%, at least 98%, at least 99%, or 100% identical to the amino acid sequence of SEQ ID NO:225 and/or a V_{L} domain that comprises an amino acid sequence that is at least 95%, at least 96%, at least 97%, at least 98%, at least 99%, or 100% identical to the amino acid sequence of SEQ ID NO:226. In some embodiments, a binding site that binds NKG2D comprises: a VH domain that comprises the amino acid sequence of SEQ ID NO:225 and/or a VH domain that comprises the amino acid sequence of SEQ ID NO:226. In some embodiments, a binding site that binds NKG2D comprises: a VH domain that comprises the amino acid sequence of SEQ ID NO:225 and a VH domain that comprises the amino acid sequence of SEQ ID NO:226. In some embodiments, a binding site that binds NKG2D comprises: a VH domain that comprises 1, 2, or all 3 CDRs from a VH domain comprising the amino acid sequence of SEQ ID NO:225 and/or a VH domain that comprises 1, 2, or all 3 CDRs from a VH domain comprising the amino acid sequence of SEQ ID NO:226.

In some embodiments, a binding site that binds NKG2D comprises: a VH domain that comprises an amino acid sequence that is at least 95%, at least 96%, at least 97%, at least 98%, at least 99%, or 100% identical to the amino acid sequence of SEQ ID NO:227 and/or a V_{L} domain that comprises an amino acid sequence that is at least 95%, at least 96%, at least 97%, at least 98%, at least 99%, or 100% identical to the amino acid sequence of SEQ ID NO:228. In some embodiments, a binding site that binds NKG2D comprises: a VH domain that comprises the amino acid sequence of SEQ ID NO:227 and/or a VH domain that comprises the amino acid sequence of SEQ ID NO:228. In some embodiments, a binding site that binds NKG2D comprises: a VH domain that comprises the amino acid sequence of SEQ ID NO:227 and a VH domain that comprises the amino acid sequence of SEQ ID NO:228. In some embodiments, a binding site that binds NKG2D comprises: a VH domain that comprises 1, 2, or all 3 CDRs from a VH domain comprising the amino acid sequence of SEQ ID NO:227 and/or a VH domain that comprises 1, 2, or all 3 CDRs from a VH domain comprising the amino acid sequence of SEQ ID NO:228.

In some embodiments, a binding site that binds NKG2D comprises: a VH domain that comprises an amino acid sequence that is at least 95%, at least 96%, at least 97%, at least 98%, at least 99%, or 100% identical to the amino acid sequence of SEQ ID NO:229 and/or a V_{L} domain that comprises an amino acid sequence that is at least 95%, at least 96%, at least 97%, at least 98%, at least 99%, or 100% identical to the amino acid sequence of SEQ ID NO:230. In some embodiments, a binding site that binds NKG2D comprises: a VH domain that comprises the amino acid sequence of SEQ ID NO:229 and/or a VH domain that comprises the amino acid sequence of SEQ ID NO:230. In some embodiments, a binding site that binds NKG2D comprises: a VH domain that comprises the amino acid sequence of SEQ ID NO:229 and a VH domain that comprises the amino acid sequence of SEQ ID NO:230. In some embodiments, a binding site that binds NKG2D comprises: a VH domain that comprises 1, 2, or all 3 CDRs from a VH domain comprising the amino acid sequence of SEQ ID NO:229 and/or a VH domain that comprises 1, 2, or all 3 CDRs from a VH domain comprising the amino acid sequence of SEQ ID NO:230.

In some embodiments, a binding site that binds NKG2D comprises: a VH domain that comprises an amino acid sequence that is at least 95%, at least 96%, at least 97%, at least 98%, at least 99%, or 100% identical to the amino acid sequence of SEQ ID NO:231 and/or a V_{L} domain that comprises an amino acid sequence that is at least 95%, at least 96%, at least 97%, at least 98%, at least 99%, or 100% identical to the amino acid sequence of SEQ ID NO:232. In some embodiments, a binding site that binds NKG2D comprises: a VH domain that comprises the amino acid sequence of SEQ ID NO:231 and/or a VH domain that comprises the amino acid sequence of SEQ ID NO:232. In some embodiments, a binding site that binds NKG2D comprises: a VH domain that comprises the amino acid sequence of SEQ ID NO:231 and a VH domain that comprises the amino acid sequence of SEQ ID NO:232. In some embodiments, a binding site that binds NKG2D comprises: a VH domain that comprises 1, 2, or all 3 CDRs from a VH domain comprising the amino acid sequence of SEQ ID NO:231 and/or a VH domain that comprises 1, 2, or all 3 CDRs from a VH domain comprising the amino acid sequence of SEQ ID NO:232.

In some embodiments, a binding site that binds NKG2D comprises: a VH domain that comprises an amino acid sequence that is at least 95%, at least 96%, at least 97%, at least 98%, at least 99%, or 100% identical to the amino acid sequence of SEQ ID NO:233 and/or a V_{L} domain that comprises an amino acid sequence that is at least 95%, at least 96%, at least 97%, at least 98%, at least 99%, or 100% identical to the amino acid sequence of SEQ ID NO:234. In some embodiments, a binding site that binds NKG2D comprises: a VH domain that comprises the amino acid sequence of SEQ ID NO:233 and/or a VH domain that comprises the amino acid sequence of SEQ ID NO:234. In some embodiments, a binding site that binds NKG2D comprises: a VH domain that comprises the amino acid sequence of SEQ ID NO:233 and a VH domain that comprises the amino acid sequence of SEQ ID NO:234. In some embodiments, a binding site that binds NKG2D comprises: a VH domain that comprises 1, 2, or all 3 CDRs from a VH domain comprising the amino acid sequence of SEQ ID NO:233 and/or a VH domain that comprises 1, 2, or all 3 CDRs from a VH domain comprising the amino acid sequence of SEQ ID NO:234.

In some embodiments, a binding site that binds NKG2D comprises: a VH domain that comprises an amino acid sequence that is at least 95%, at least 96%, at least 97%, at least 98%, at least 99%, or 100% identical to the amino acid sequence of SEQ ID NO:235 and/or a V_{L} domain that comprises an amino acid sequence that is at least 95%, at least 96%, at least 97%, at least 98%, at least 99%, or 100% identical to the amino acid sequence of SEQ ID NO:236. In some embodiments, a binding site that binds NKG2D comprises: a VH domain that comprises the amino acid sequence of SEQ ID NO:235 and/or a VH domain that comprises the amino acid sequence of SEQ ID NO:236. In some embodiments, a binding site that binds NKG2D comprises: a VH domain that comprises the amino acid sequence of SEQ ID NO:235 and a VH domain that comprises the amino acid sequence of SEQ ID NO:236. In some embodiments, a binding site that binds NKG2D comprises: a VH domain that comprises 1, 2, or all 3 CDRs from a VH domain comprising the amino acid sequence of SEQ ID NO:235 and/or a VH domain that comprises 1, 2, or all 3 CDRs from a VH domain comprising the amino acid sequence of SEQ ID NO:236.

In some embodiments, a binding site that binds NKG2D comprises: a VH domain that comprises an amino acid sequence that is at least 95%, at least 96%, at least 97%, at least 98%, at least 99%, or 100% identical to the amino acid sequence of SEQ ID NO:237 and/or a V_{L} domain that comprises an amino acid sequence that is at least 95%, at least 96%, at least 97%, at least 98%, at least 99%, or 100% identical to the amino acid sequence of SEQ ID NO:238. In some embodiments, a binding site that binds NKG2D comprises: a VH domain that comprises the amino acid sequence of SEQ ID NO:237 and/or a VH domain that comprises the amino acid sequence of SEQ ID NO:238. In some embodiments, a binding site that binds NKG2D comprises: a VH domain that comprises the amino acid sequence of SEQ ID NO:237 and a VH domain that comprises the amino acid sequence of SEQ ID NO:238. In some embodiments, a binding site that binds NKG2D comprises: a VH domain that comprises 1, 2, or all 3 CDRs from a VH domain comprising the amino acid sequence of SEQ ID NO:237 and/or a VH domain that comprises 1, 2, or all 3 CDRs from a VH domain comprising the amino acid sequence of SEQ ID NO:238.

In some embodiments, a binding site that binds NKG2D comprises: a VH domain that comprises an amino acid sequence that is at least 95%, at least 96%, at least 97%, at least 98%, at least 99%, or 100% identical to the amino acid sequence of SEQ ID NO:239 and/or a V_{L} domain that comprises an amino acid sequence that is at least 95%, at least 96%, at least 97%, at least 98%, at least 99%, or 100% identical to the amino acid sequence of SEQ ID NO:238. In some embodiments, a binding site that binds NKG2D comprises: a VH domain that comprises the amino acid sequence of SEQ ID NO:239 and/or a VH domain that comprises the amino acid sequence of SEQ ID NO:238. In some embodiments, a binding site that binds NKG2D comprises: a VH domain that comprises the amino acid sequence of SEQ ID NO:239 and a VH domain that comprises the amino acid sequence of SEQ ID NO:238. In some embodiments, a binding site that binds NKG2D comprises: a VH domain that comprises 1, 2, or all 3 CDRs from a VH domain comprising the amino acid sequence of SEQ ID NO:239 and/or a VH domain that comprises 1, 2, or all 3 CDRs from a VH domain comprising the amino acid sequence of SEQ ID NO:238.

In some embodiments, a binding site that binds NKG2D comprises: a VH domain that comprises an amino acid sequence that is at least 95%, at least 96%, at least 97%, at least 98%, at least 99%, or 100% identical to the amino acid sequence of SEQ ID NO:240 and/or a V_{L} domain that comprises an amino acid sequence that is at least 95%, at least 96%, at least 97%, at least 98%, at least 99%, or 100% identical to the amino acid sequence of SEQ ID NO:241. In some embodiments, a binding site that binds NKG2D comprises: a VH domain that comprises the amino acid sequence of SEQ ID NO:240 and/or a VH domain that comprises the amino acid sequence of SEQ ID NO:241. In some embodiments, a binding site that binds NKG2D comprises: a VH domain that comprises the amino acid sequence of SEQ ID NO:240 and a VH domain that comprises the amino acid sequence of SEQ ID NO:241. In some embodiments, a binding site that binds NKG2D comprises: a VH domain that comprises 1, 2, or all 3 CDRs from a VH domain comprising the amino acid sequence of SEQ ID NO:240 and/or a VH domain that comprises 1, 2, or all 3 CDRs from a VH domain comprising the amino acid sequence of SEQ ID NO:241.

Sequences of exemplary anti-NKG2D antigen binding sites are provided in Table 49. In some embodiments, a binding protein comprising an anti-NKG2D antigen binding site of the present disclosure comprises 1, 2, 3, 4, 5, or all 6 CDR sequences of an anti-NKG2D antibody described in Table 49. In some embodiments, a binding protein comprising an anti-NKG2D antigen binding site of the present disclosure comprises a VH domain sequence and/or V_{L} domain sequence of an anti-NKG2D antibody described in Table 49.

TABLE 49
Anti-NKG2D binding protein sequences.

| | | SEQ |
|---|---|---|
| Name | Sequence | ID NO |
| CL- | GYTFTDYY | 157 |
| 485789 | | |
| CDR1- | | |
| H1 | | |
| CL- | VDPEDGET | 158 |
| 485789 | | |
| CDR1- | | |
| H2 | | |
| CL- | ARGIARYPNGPGGLLYYYYYGMDV | 159 |
| 485789 | | |
| CDR1- | | |
| H3 | | |
| CL- | NIGSKN | 160 |
| 485789 | | |
| CDR1- | | |
| L1 | | |
| CL- | RDS | N/A |
| 485789 | | |
| CDR1- | | |
| L2 | | |
| CL- | QVWDSFAFV | 162 |
| 485789 | | |
| CDR1- | | |
| L3 | | |
| CL- | GFTFSSYA | 163 |
| 485797 | | |
| CDR1- | | |
| H1 | | |
| CL- | ISYDGSNK | 164 |
| 485797 | | |
| CDR1- | | |
| H2 | | |
| CL- | ARYSNYWWFDY | 165 |
| 485797 | | |
| CDR1- | | |
| H3 | | |
| CL- | SSNIGNNA | 166 |
| 485797 | | |
| CDR1- | | |
| L1 | | |
| CL- | YDD | N/A |
| 485797 | | |
| CDR1- | | |
| L2 | | |
| CL- | AAWDDSLSGVV | 168 |
| 485797 | | |
| CDR1- | | |
| L3 | | |
| CL- | GFTFSSYA | 169 |
| 485845 | | |
| CDR1- | | |
| H1 | | |
| CL- | ISYDGSNK | 170 |
| 485845 | | |
| CDR1- | | |
| H2 | | |
| CL- | ARYDNTRIFDY | 171 |
| 485845 | | |
| CDR1- | | |
| H3 | | |
| CL- | SSNIGNNA | 172 |
| 485845 | | |
| CDR1- | | |
| L1 | | |
| CL- | YDD | N/A |
| 485845 | | |
| CDR1- | | |
| L2 | | |
| CL- | AAWDDSLNGYV | 174 |
| 485845 | | |
| CDR1- | | |
| L3 | | |
| CL- | GFTFSSYA | 175 |
| 486097 | | |
| CDR1- | | |
| H1 | | |
| ICL- | ISYDGSNK | 176 |
| 486097 | | |
| CDR1- | | |
| H2 | | |
| CL- | ARLPWVGSVDY | 177 |
| 486097 | | |
| CDR1- | | |
| H3 | | |
| ICL- | SSNIGNNA | 178 |
| 486097 | | |
| CDR1- | | |
| L1 | | |
| CL- | YDD | N/A |
| 486097 | | |
| CDR1- | | |
| L2 | | |
| CL- | AAWDDSLFGWV | 180 |
| 486097 | | |
| CDR1- | | |
| L3 | | |
| CL- | GFSLSTSGVG | 181 |
| 498027 | | |
| CDR1- | | |
| H1 | | |
| CL- | IYWDDDK | 182 |
| 498027 | | |
| CDR1- | | |
| H2 | | |
| CL- | AHRTGIAVAGIAFDI | 183 |
| 498027 | | |
| CDR1- | | |
| H3 | | |
| CL- | KLGDKY | 184 |
| 498027 | | |
| CDR1- | | |
| L1 | | |
| CL- | QDT | N/A |
| 498027 | | |
| CDR1- | | |
| L2 | | |
| CL- | QAWDSSSLYV | 186 |
| 498027 | | |
| CDR1- | | |
| L3 | | |
| CL- | GGSISGSSYY | 187 |
| 523781 | | |
| CDR1- | | |
| H1 | | |
| CL- | IYSSGST | 188 |
| 523781 | | |
| CDR1- | | |
| H2 | | |
| CL- | AKPLLRDYYFYGVDV | 189 |
| 523781 | | |
| CDR1- | | |
| H3 | | |
| CL- | QAISSY | 190 |
| 523781 | | |
| CDR1- | | |
| L1 | | |
| CL- | PAS | N/A |
| 523781 | | |
| CDR1- | | |
| L2 | | |
| CL- | QQLKSYPWT | 192 |
| 523781 | | |
| CDR1- | | |
| L3 | | |
| CL- | GFTFSSYA | 193 |
| 524785 | | |
| CDR1- | | |
| H1 | | |
| CL- | ISESGSST | 194 |
| 524785 | | |
| CDR1- | | |
| H2 | | |
| CL- | AKDRWFEGYHSYYGMDV | 195 |
| 524785 | | |
| CDR1- | | |
| H3 | | |
| CL- | QGISNN | 196 |
| 524785 | | |
| CDR1- | | |
| L1 | | |
| CL- | ATS | N/A |
| 524785 | | |
| CDR1- | | |
| L2 | | |
| CL- | QQLKSYPSFT | 198 |
| 524785 | | |
| CDR1- | | |
| L3 | | |
| CL-526201 | GGSISSSSYY | 199 |
| CDR-H1 | | |
| CL-526201 | IYSSGST | 200 |
| CDR-H2 | | |
| CL-526201 | AKPLLRDYYYYGVDV | 201 |
| CDR-H3 | | |
| CL-526201 | QAISSY | 202 |
| CDR-L1 | | |
| CL-526201 | PAS | N/A |
| CDR-L2 | | |
| CL-526201 | QQLNSFPWT | 204 |
| CDR-L3 | | |
| CL-600412 | GYTFTTYG | 205 |
| CDR-H1 | | |
| CL-600412 | ISVFNGSP | 206 |
| CDR-H2 | | |
| CL-600412 | ARGGTGDYYPLFDY | 207 |
| CDR-H3 | | |
| ICL-600412 | QGIRND | 208 |
| CDR-L1 | | |
| ICL-600412 | AAS | N/A |
| CDR-L2 | | |
| CL-600412 | LQDYNYPYT | 210 |
| CDR-L3 | | |
| CL-607768 | GGSITSTSYF | 211 |
| CDR-H1 | | |
| CL-607768 | IYHSGST | 212 |
| CDR-H2 | | |
| ICL-607768 | ARHEFGIRDAFDI | 213 |
| CDR-H3 | | |
| CL-607768 | QSIGNN | 214 |
| CDR-L1 | | |
| CL-607768 | YAS | N/A |
| CDR-L2 | | |
| CL-607768 | HQSNSFPFT | 216 |
| CDR-L3 | | |
| CL- | EVOLVQSGAEVKKPGATVKISCKVSGYTFTDYYMHWVQQAPGKGLEWMGLVDPEDGETIYAEKFQGRVTITADTST | 217 |
| 485789 | DTAYMELSSLRSEDTAVYYCARGIARYPNGPGGLLYYYYYGMDVWGQGTTVTVSS | |
| VH | | |
| CL- | SYELTQPLSVSVALGQTARITCGGNNIGSKNVHWYQQKPGQAPVLVIYRDSNRPSGIPERFSGSNSGNTATLTISRAQA | 218 |
| 485789 | GDEADYYCQVWDSFAFVFGGGTKLTVL | |
| VL | | |
| ICL- | QVQLVESGGGVVQPGRSLRLSCAASGFTFSSYAMHWVRQAPGKGLEWVAVISYDGSNKYYADSVKGRFTISRDNSK | 219 |
| 485797 | NTLYLQMNSLRAEDTAVYYCARYSNYWWFDYWGQGTLVTVSS | |
| VH | | |
| CL- | QSVLTQPPSVSEAPRQRVTISCSGSSSNIGNNAVNWYQQLPGKAPKLLIYYDDLLPSGVSDRFSGSKSGTSASLAISGLQ | 220 |
| 485797 | SEDEADYYCAAWDDSLSGVVFGGGTKLTVL | |
| VL | | |
| CL- | QVQLVESGGGVVQPGRSLRLSCAASGFTFSSYAMHWVRQAPGKGLEWVAVISYDGSNKYYADSVKGRFTISRDNSK | 221 |
| 485845 | NTLYLQMNSLRAEDTAVYYCARYDNTRIFDYWGQGTLVTVSS | |
| VH | | |
| CL- | QSVLTQPPSVSEAPRQRVTISCSGSSSNIGNNAVNWYQQLPGKAPKLLIYYDDLLPSGVSDRFSGSKSGTSASLAISGLQ | 222 |
| 485845 | SEDEADYYCAAWDDSLNGYVFGGGTKLTVL | |
| VL | | |
| CL- | QVQLVESGGGVVQPGRSLRLSCAASGFTFSSYAMHWVRQAPGKGLEWVAVISYDGSNKYYADSVKGRFTISRDNSK | 223 |
| 486097 | NTLYLQMNSLRAEDTAVYYCARLPWVGSVDYWGQGTLVTVSS | |
| VH | | |
| CL- | QSVLTQPPSVSEAPRQRVTISCSGSSSNIGNNAVNWYQQLPGKAPKLLIYYDDLLPSGVSDRFSGSKSGTSASLAISGLQ | 224 |
| 486097 | SEDEADYYCAAWDDSLFGWVFGGGTKLTVL | |
| VL | | |
| CL- | QVTLKESGPTLVKPTQTLTLTCTFSGFSLSTSGVGVGWIRQPPGKALEWLALIYWDDDKRYSPSLESRLTITKDTSKNQV | 225 |
| 498027 | VLTMTNMDPVDTATYYCAHRTGIAVAGIAFDIWGQGTMVTVSS | |
| VH | | |
| CL- | QSVLTQPPSVSVSPGQTASITCSGDKLGDKYVSWYQQKPGQSPVLVIYQDTKRPSGIPERFSGSNSGNTATLTISGTQT | 226 |
| 498027 | VDEADYYCQAWDSSSLYVFGTGTKVTVL | |
| VL | | |
| CL- | QLQLQESGPGLVKPSETLSLTCTVSGGSISGSSYYWVWIRQPPGKGLEWIGNIYSSGSTYYNPSFKSRVIISVDTSKSQFS | 227 |
| 523781 | LKLNSVTAADTAVYYCAKPLLRDYYFYGVDVWGQGTTVTVSS | |
| VH | | |
| CL- | DIQLTPSPSFLSASVGDRVTITCRASQAISSYLAWYQQKPGKAPKLLISPASTLQSGVPSRFSGSGSGTEFTLTISSLOPED | 228 |
| 523781 | FATYYCQQLKSYPWTFGQGTKVEIK | |
| VL | | |
| CL- | EVQLLESGGGLVQPGGSLRLSCTASGFTFSSYAMSWVRQAPGKGLEWVSLISESGSSTYYADSVKGRFTNSRDDSRNT | 229 |
| 524785 | LYLQMNSLRAEDTAVYYCAKDRWFEGYHSYYGMDVWGQGTTVTVSS | |
| VH | | |
| CL- | DIQLTQSPSFLSAFVGDRVTITCRASQGISNNLAWYQQKPGKAPKLLVYATSTLQSGVPSRFSGSGSGTEFTLTVSSLQP | 230 |
| 524785 | EDFATYYCQQLKSYPSFTFGPGTKVDIK | |
| VL | | |
| CL- | QLQLQESGPGLVKPSETLSLTCTVSGGSISSSSYYWVWIRQSPGKGLEWIGNIYSSGSTHYNPSLKSRVIISVDTSKNQFS | 231 |
| 526201 | LKLKSVTAADTAVYYCAKPLLRDYYYYGVDVWGQGTTVTVSS | |
| VH | | |
| CL- | DIQLTPSPSFLSASVGDRVTITCRASQAISSYLAWYQQKPGKAPKLLISPASTLQSGVPSRFSGSGSGTEFTLTISSLOPED | 232 |
| 526201 | FATYFCQQLNSFPWTFGQGTKVEIK | |
| VL | | |
| CL- | QVQLQQSGTEVKKPGASVKVSCKASGYTFTTYGLTWIRQAPGQGLEWMGWISVFNGSPNYSLKFQGRVTMTADTF | 233 |
| 600412 | TNTAYMELRSLRSDDTAVYYCARGGTGDYYPLFDYWGQGTLVTVSS | |
| VH | | |
| CL- | AIRMTQSPSSLSASVGDRVTITCRASQGIRNDLGWYQQKPGKAPKLLIFAASNLESGVPSRFSGSRFGTDFTLTISSLQP | 234 |
| 600412 | EDFATYYCLQDYNYPYTFGQGTKLEIK | |
| VL | | |
| CL- | QVQLLESGPGLVKPAETLSLTCTVSGGSITSTSYFWDWIRQPPGKGLEWIGSIYHSGSTYYDSSLKTRVTISVDTSKYQFS | 235 |
| 607768 | LRLSSVTAADTAVYYCARHEFGIRDAFDIWGQGTLVTVSS | |
| VH | | |
| CL- | EIVMTQSPDFQSVTPKEKVTITCRASQSIGNNLHWYQQKPDQSPKLLIKYASQSISGVPSRFSGSGFGTDFTLTINSLEA | 236 |
| 607768 | EDAAAYYCHQSNSFPFTFGPGTKVEIK | |
| VL | | |
| CL- | EVQLLESGGGLVQPGGSLRLSCTASGFTFSSYAMSWVREAPGKGLEWVSLISESGSSTYYAESVKGRFTISRDDSKNT | 237 |
| 524785 | LYLQMNSLRAEDTAVYYCAKDRWFEGYHSYYGMDVWGQGTTVTVSS | |
| _D62E | | |
| VH | | |
| (CM2) | | |
| ICL- | QPEDFATYYCQQLKSYPSFTFGPGTKVDIK | 238 |
| 524785 | DIQLTQSPSFLSAFVGDRVTITCRASQGISNNLAWYQKKPGKAPKLLVYATSTLQSGVPSRFSGSGSGTEFTLTVSSL | |
| _D62E, | | |
| CL-524785 | | |
| V1.5 VL | | |
| (CM2) | | |
| CL- | EVQLLESGGGLVQPGGSLRLSCTASGFTFSSYAMSWVREAPGKGLEWVSLISESGSSTYYAESVKGRFTNSRDDSRN | 239 |
| 524785 | TLYLQMNSLRAEDTAVYYCAKDRWFEGYHSYYGMDVWGQGTTVTVSS | |
| V1.5 | | |
| VH | | |
| (D62E, | | |
| N701, | | |
| R76K) | | |
| (CM2) | | |
| CL- | QLQLQESGPGLVKPSETLSLTCTVSGGSISSSSYYWVWIRESPGKGLEWIGNIYSSGSTHYNPSLKSRVIISVGTSKNQ | 240 |
| 526201 | FSLKLKSVTAADTAVYYCAKPLLRDYYYYGVDVWGQGTTVTVSS | |
| D74G | | |
| VH | | |
| (CM2) | | |
| CL- | DIQLTPSPSFLSASVGDRVTITCRASQAISSYLAWYQKKPGKAPKLLISPASTLQSGVPSRFSGSGSGTEFTLTISSLOPE | 241 |
| 526201 | DFATYFCQQLNSFPWTFGQGTKVEIK | |
| D74G | | |
| VL | | |
| (CM2) | | |

### Chain Pairing

In some embodiments, a binding protein of the present disclosure may comprise one or more amino acid substitutions, e.g., within a VH and/or VL domain, to facilitate heavy-light chain pairing. For example, a binding protein such as a bispecific, trispecific, or multispecific binding protein of the present disclosure comprising two or more distinct species of heavy and cognate light chains may comprise one or more substitutions in the pairing heavy and light chains (e.g., in the VH and VL domains) that promote proper pairing of the correct, cognate heavy and light chains. One example of such technology is the DuetMab concept, in which native disulfide bonds in only one arm of a bispecific antibody are removed and replaced by non-natural disulfide bonds at different positions (e.g., through reciprocal removal of native cysteine residues from the heavy and light chain and introduction of non-native cysteine residues at other positions in the heavy and light chain). See, e.g., Mazor, Y. et al. (2015) MAbs 7(2):377-389.

In some embodiments, a binding protein of the present disclosure may comprise pair of reciprocal amino acid substitutions on the VH and VL domains that facilitate heavy-light chain pairing. In some embodiments, one, two, or all three antigen binding domains of a trispecific binding protein of the present disclosure may comprise pair of reciprocal amino acid substitutions on the VH and VL domains that facilitate heavy-light chain pairing. In some embodiments, one or both antigen binding domain(s) of a bispecific binding protein of the present disclosure may comprise pair of reciprocal amino acid substitutions on the VH and VL domains that facilitate heavy-light chain pairing. Amino acid substitutions of V_{H} and V_{L} domains that facilitate heavy-light chain pairing (e.g., promote correct heavy-light chain pairing) are known in the art; see, e.g., Dillon, M. et al. (2017) MAbs 9(2):213-230 and Bagert, J. D. et al. (2023) MAbs 15(1):2273449.

### Linkers

In some embodiments, the linkers L₁, L₂, L₃, and L₄ range from no amino acids (length=0) to about 100 amino acids long, or less than 100, 50, 40, 30, 20, or 15 amino acids or less. The linkers can also be 10, 9, 8, 7, 6, 5, 4, 3, 2, or 1 amino acids long. L₁, L₂, L₃, and L₄ in one binding protein may all have the same amino acid sequence or may all have different amino acid sequences.

Examples of suitable linkers include, for example, GGGGSGGGGS (SEQ ID NO:102), GGGGSGGGGSGGGGS (SEQ ID NO: 103), S, RT, TKGPS (SEQ ID NO: 104), GQPKAAP (SEQ ID NO: 105), GGSGSSGSGG (SEQ ID NO: 106), and DKTHT (SEQ ID NO: 109), as well as those disclosed in International Publication Nos. WO2017/074878, WO2017/180913, and WO2020/210392. The examples listed above are not intended to limit the scope of the disclosure in any way, and linkers comprising randomly selected amino acids selected from the group consisting of valine, leucine, isoleucine, serine, threonine, lysine, arginine, histidine, aspartate, glutamate, asparagine, glutamine, glycine, and proline have been shown to be suitable in the binding proteins.

The identity and sequence of amino acid residues in the linker may vary depending on the type of secondary structural element necessary to achieve in the linker. For example, glycine, serine, and alanine are best for linkers having maximum flexibility. Some combination of glycine, proline, threonine, and serine are useful if a more rigid and extended linker is necessary. Any amino acid residue may be considered as a linker in combination with other amino acid residues to construct larger peptide linkers as necessary depending on the desired properties.

In some embodiments, the length of L₁ is at least twice the length of L₃. In some embodiments, the length of L₂ is at least twice the length of L₄. In some embodiments, the length of L₁ is at least twice the length of L₃, and the length of L₂ is at least twice the length of L₄. In some embodiments, L₁ is 3 to 12 amino acid residues in length, L₂ is 3 to 14 amino acid residues in length, L₃ is 1 to 8 amino acid residues in length, and L₄ is 1 to 3 amino acid residues in length. In some embodiments, L₁ is 5 to 10 amino acid residues in length, L₂ is 5 to 8 amino acid residues in length, L₃ is 1 to 5 amino acid residues in length, and L₄ is 1 to 2 amino acid residues in length. In some embodiments, L₁ is 7 amino acid residues in length, L₂ is 5 amino acid residues in length, L₃ is 1 amino acid residue in length, and L₄ is 2 amino acid residues in length.

In some embodiments, L₁, L₂, L₃ and L₄ each independently are zero amino acids in length or comprise a sequence selected from the group consisting of GGGGSGGGGS (SEQ ID NO:102), GGGGSGGGGSGGGGS (SEQ ID NO: 103), S, RT, TKGPS (SEQ ID NO: 104), GQPKAAP (SEQ ID NO: 105), and GGSGSSGSGG (SEQ ID NO: 106). In some embodiments, L₁, L₂, L₃ and L₄ each independently comprise a sequence selected from the group consisting of GGGGSGGGGS (SEQ ID NO:102), GGGGSGGGGSGGGGS (SEQ ID NO: 103), S, RT, TKGPS (SEQ ID NO: 104), GQPKAAP (SEQ ID NO: 105), and GGSGSSGSGG (SEQ ID NO: 106). In some embodiments, L₁ comprises the sequence GQPKAAP (SEQ ID NO: 105), L₂ comprises the sequence TKGPS (SEQ ID NO:104), L₃ comprises the sequence S, and L₄ comprises the sequence RT.

In some embodiments, at least one of L₁, L₂, L₃ or L₄ comprises the sequence DKTHT (SEQ ID NO:109). In some embodiments, L₁, L₂, L₃ and L₄ comprise the sequence DKTHT (SEQ ID NO:109).

### Fc Regions and Constant Domains

In some embodiments, a binding protein of the present disclosure comprises a second polypeptide chain further comprising an Fc region linked to C_{H1}, the Fc region comprising an immunoglobulin hinge region and C_{H2} and C_{H3} immunoglobulin heavy chain constant domains. In some embodiments, a binding protein of the present disclosure comprises a third polypeptide chain further comprising an Fc region linked to C_{H1}, the Fc region comprising an immunoglobulin hinge region and C_{H2} and C_{H3} immunoglobulin heavy chain constant domains. In some embodiments, a binding protein of the present disclosure comprises a second polypeptide chain further comprising an Fc region linked to C_{H1}, the Fc region comprising an immunoglobulin hinge region and C_{H2} and C_{H3} immunoglobulin heavy chain constant domains, and a third polypeptide chain further comprising an Fc region linked to C_{H1}, the Fc region comprising an immunoglobulin hinge region and C_{H2} and C_{H3} immunoglobulin heavy chain constant domains.

In some embodiments, a binding protein of the present disclosure comprises a full-length antibody heavy chain or a polypeptide chain comprising an Fc region. In some embodiments, the Fc region is a human Fc region, e.g., a human IgG1, IgG2, IgG3, or IgG4 Fc region. In some embodiments, the antibody constant region includes an antibody hinge, C_{H1}, C_{H2}, C_{H3}, and optionally C_{H4} domains. In some embodiments, the Fc region includes antibody C_{H2}, C_{H3}, and optionally C_{H4} domains. In some embodiments, the Fc region is a human IgG1 Fc region. In some embodiments, the Fc region is a human IgG4 Fc region. In some embodiments, the Fc region includes one or more of the mutations described infra. In some embodiments, the Fc region is an Fc region of one of the heavy chain polypeptides (e.g., polypeptide 2 or 3) of a binding protein shown in Table 4. In some embodiments, the heavy chain constant region is a constant region of one of the heavy chain polypeptides (e.g., polypeptide 2 or 3) of a binding protein shown in Table 4. In some embodiments, the light chain constant region is a constant region of one of the light chain polypeptides (e.g., polypeptide 1 or 4) of a binding protein shown in Table 4.

In some embodiments, a binding protein of the present disclosure includes one or two Fc variants. The term “Fc variant” as used herein refers to a molecule or sequence that is modified from a native Fc but still comprises a binding site for the salvage receptor, FcRn (neonatal Fc receptor). Exemplary Fc variants, and their interaction with the salvage receptor, are known in the art. Thus, the term “Fc variant” can comprise a molecule or sequence that is humanized from a non-human native Fc. Furthermore, a native Fc comprises regions that can be removed because they provide structural features or biological activity that are not required for the antibody-like binding proteins of the invention. Thus, the term “Fc variant” comprises a molecule or sequence that lacks one or more native Fc sites or residues, or in which one or more Fc sites or residues has be modified, that affect or are involved in: (1) disulfide bond formation, (2) incompatibility with a selected host cell, (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

In some embodiments, a binding protein of the present disclosure (e.g., a trispecific binding protein) comprises a “knob” mutation on the second polypeptide chain and a “hole” mutation on the third polypeptide chain. In some embodiments, a binding protein of the present disclosure comprises a “knob” mutation on the third polypeptide chain and a “hole” mutation on the second polypeptide chain. In some embodiments, the “knob” mutation comprises substitution(s) at positions corresponding to positions 354 and/or 366 of human IgG1 or IgG4 according to EU Index. In some embodiments, the amino acid substitutions are S354C, T366W, T366Y, S354C and T366W, or S354C and T366Y. In some embodiments, the “knob” mutation comprises substitutions at positions corresponding to positions 354 and 366 of human IgG1 or IgG4 according to EU Index. In some embodiments, the amino acid substitutions are S354C and T366W. In some embodiments, the “hole” mutation comprises substitution(s) at positions corresponding to positions 407 and, optionally, 349, 366, and/or 368 and of human IgG1 or IgG4 according to EU Index. In some embodiments, the amino acid substitutions are Y407V or Y407T and optionally Y349C, T366S, and/or L368A. In some embodiments, the “hole” mutation comprises substitutions at positions corresponding to positions 349, 366, 368, and 407 of human IgG1 or IgG4 according to EU Index. In some embodiments, the amino acid substitutions are Y349C, T366S, L368A, and Y407V.

In some embodiments, the second polypeptide chain further comprises a first Fc region linked to CH1, the first Fc region comprising an immunoglobulin hinge region and C_{H2} and C_{H3} immunoglobulin heavy chain constant domains, wherein the first Fc region comprises amino acid substitution(s) at positions corresponding to positions 366 and optionally 354 of human IgG1 or IgG4 according to EU Index, wherein the amino acid substitutions are T366W or T366Y and optionally S354C; and wherein the third polypeptide chain further comprises a second Fc region linked to CH1, the second Fc region comprising an immunoglobulin hinge region and C_{H2} and C_{H3} immunoglobulin heavy chain constant domains, wherein the second Fc region comprises amino acid substitution(s) at positions corresponding to positions 407 and optionally 349, 366, and/or 368 and of human IgG1 or IgG4 according to EU Index, wherein the amino acid substitutions are Y407V or Y407T and optionally Y349C, T366S, and/or L368A.

In some embodiments, the second polypeptide chain further comprises a first Fc region linked to CH1, the first Fc region comprising an immunoglobulin hinge region and C_{H2} and CH3 immunoglobulin heavy chain constant domains, wherein the first Fc region comprises amino acid substitution(s) at positions corresponding to positions 407 and optionally 349, 366, and/or 368 and of human IgG1 or IgG4 according to EU Index, wherein the amino acid substitutions are Y407V or Y407T and optionally Y349C, T366S, and/or L368A; and wherein the third polypeptide chain further comprises a second Fc region linked to CH1, the second Fc region comprising an immunoglobulin hinge region and CH2 and CH3 immunoglobulin heavy chain constant domains, wherein the second Fc region comprises amino acid substitution(s) at positions corresponding to positions 366 and optionally 354 of human IgG1 or IgG4 according to EU Index, wherein the amino acid substitutions are T366W or T366Y and optionally S354C.

In some embodiments, the second polypeptide chain further comprises a first Fc region linked to CH1, the first Fc region comprising an immunoglobulin hinge region and CH2 and CH3 immunoglobulin heavy chain constant domains, wherein the first Fc region comprises amino acid substitution at position corresponding to position 366 of human IgG1 or IgG4 according to EU Index, wherein the amino acid substitution is T366W; and wherein the third polypeptide chain further comprises a second Fc region linked to CH1, the second Fc region comprising an immunoglobulin hinge region and CH2 and CH3 immunoglobulin heavy chain constant domains, wherein the second Fc region comprises amino acid substitution(s) at positions corresponding to positions 366, 368, and/or 407 and of human IgG1 or IgG4 according to EU Index, wherein the amino acid substitutions are T366S, L368A, and/or Y407V.

In some embodiments, the second polypeptide chain further comprises a first Fc region linked to CH1, the first Fc region comprising an immunoglobulin hinge region and CH2 and CH3 immunoglobulin heavy chain constant domains, wherein the first Fc region comprises amino acid substitution(s) at positions corresponding to positions 366, 368, and/or 407 and of human IgG1 or IgG4 according to EU Index, wherein the amino acid substitutions are T366S, L368A, and/or Y407V; and wherein the third polypeptide chain further comprises a second Fc region linked to CH1, the second Fc region comprising an immunoglobulin hinge region and CH2 and C_{H3} immunoglobulin heavy chain constant domains, wherein the second Fc region comprises amino acid substitution at position corresponding to position 366 of human IgG1 or IgG4 according to EU Index, wherein the amino acid substitution is T366W.

In some embodiments, the second polypeptide chain further comprises a first Fc region linked to CH1, the first Fc region comprising an immunoglobulin hinge region and CH2 and C_{H3} immunoglobulin heavy chain constant domains, wherein the first Fc region comprises amino acid substitutions at positions corresponding to positions 354 and 366 of human IgG1 or IgG4 according to EU Index, wherein the amino acid substitutions are S354C and T366W; and wherein the third polypeptide chain further comprises a second Fc region linked to CH1, the second Fc region comprising an immunoglobulin hinge region and CH2 and CH3 immunoglobulin heavy chain constant domains, wherein the second Fc region comprises amino acid substitutions at positions corresponding to positions 349, 366, 368, and 407 of human IgG1 or IgG4 according to EU Index, wherein the amino acid substitutions are Y349C, T366S, L368A, and Y407V. In some embodiments, the second polypeptide chain further comprises a first Fc region linked to CH1, the first Fc region comprising an immunoglobulin hinge region and CH2 and CH3 immunoglobulin heavy chain constant domains, wherein the first Fc region comprises amino acid substitutions at positions corresponding to positions 349, 366, 368, and 407 of human IgG1 or IgG4 according to EU Index, wherein the amino acid substitutions are Y349C, T366S, L368A, and Y407V; and wherein the third polypeptide chain further comprises a second Fc region linked to CH1, the second Fc region comprising an immunoglobulin hinge region and CH2 and CH3 immunoglobulin heavy chain constant domains, wherein the second Fc region comprises amino acid substitutions at positions corresponding to positions 354 and 366 of human IgG1 or IgG4 according to EU Index, wherein the amino acid substitutions are S354C and T366W. In some embodiments, the first and/or second Fc regions are human IgG1 Fc regions. In some embodiments, the first and/or second Fc regions are human IgG4 Fc regions.

In some embodiments, the second polypeptide chain further comprises a first Fc region linked to CH1, wherein the first Fc region is a human IgG4 Fc region comprising an immunoglobulin hinge region and CH2 and CH3 immunoglobulin heavy chain constant domains, wherein the first Fc region comprises amino acid substitutions at positions corresponding to positions 228, 354, 366, and 409 of human IgG4 according to EU Index, wherein the amino acid substitutions are S228P, S354C, T366W, and R409K; and wherein the third polypeptide chain further comprises a second Fc region linked to CH1, wherein the second Fc region is a human IgG4 Fc region comprising an immunoglobulin hinge region and CH2 and CH3 immunoglobulin heavy chain constant domains, wherein the second Fc region comprises amino acid substitutions at positions corresponding to positions 228, 349, 366, 368, 407, and 409 of human IgG4 according to EU Index, wherein the amino acid substitutions are S228P, Y349C, T366S, L368A, Y407V, and R409K. In some embodiments, the second polypeptide chain further comprises a first Fc region linked to CH1, wherein the first Fc region is a human IgG4 Fc region comprising an immunoglobulin hinge region and CH2 and CH3 immunoglobulin heavy chain constant domains, wherein the first Fc region comprises amino acid substitutions at positions corresponding to positions 228, 349, 366, 368, 407, and 409 of human IgG4 according to EU Index, wherein the amino acid substitutions are S228P, Y349C, T366S, L368A, Y407V, and R409K; and wherein the third polypeptide chain further comprises a second Fc region linked to CH1, wherein the second Fc region is a human IgG4 Fc region comprising an immunoglobulin hinge region and CH2 and CH3 immunoglobulin heavy chain constant domains, wherein the second Fc region comprises amino acid substitutions at positions corresponding to positions 228, 354, 366, and 409 of human IgG4 according to EU Index, wherein the amino acid substitutions are S228P, S354C, T366W, and R409K.

In some embodiments, the second polypeptide chain further comprises a first Fc region linked to CH1, wherein the first Fc region is a human IgG4 Fc region comprising an immunoglobulin hinge region and CH2 and CH3 immunoglobulin heavy chain constant domains, wherein the first Fc region comprises amino acid substitutions at positions corresponding to positions 234, 235, 354, and 366 of human IgG4 according to EU Index, wherein the amino acid substitutions are F234A, L235A, S354C, and T366W; and wherein the third polypeptide chain further comprises a second Fc region linked to CH1, wherein the second Fc region is a human IgG4 Fc region comprising an immunoglobulin hinge region and CH2 and CH3 immunoglobulin heavy chain constant domains, wherein the second Fc region comprises amino acid substitutions at positions corresponding to positions 234, 235, 349, 366, 368, and 407 of human IgG4 according to EU Index, wherein the amino acid substitutions are F234A, L235A, Y349C, T366S, L368A, and Y407V. In some embodiments, the second polypeptide chain further comprises a first Fc region linked to CH1, wherein the first Fc region is a human IgG4 Fc region comprising an immunoglobulin hinge region and CH2 and CH3 immunoglobulin heavy chain constant domains, wherein the first Fc region comprises amino acid substitutions at positions corresponding to positions 234, 235, 349, 366, 368, and 407 of human IgG4 according to EU Index, wherein the amino acid substitutions are F234A, L235A, Y349C, T366S, L368A, and Y407V; and wherein the third polypeptide chain further comprises a second Fc region linked to CH1, wherein the second Fc region is a human IgG4 Fc region comprising an immunoglobulin hinge region and CH2 and CH3 immunoglobulin heavy chain constant domains, wherein the second Fc region comprises amino acid substitutions at positions corresponding to positions 234, 235, 354, and 366 of human IgG4 according to EU Index, wherein the amino acid substitutions are F234A, L235A, S354C, and T366W.

In some embodiments, a binding protein of the present disclosure comprises one or more mutations to reduce effector function, e.g., Fc receptor-mediated antibody-dependent cellular phagocytosis (ADCP), complement-dependent cytotoxicity (CDC), and/or antibody-dependent cellular cytotoxicity (ADCC). In some embodiments, the second polypeptide chain further comprises a first Fc region linked to C_{H1}, the first Fc region comprising an immunoglobulin hinge region and C_{H2} and C_{H3} immunoglobulin heavy chain constant domains; wherein the third polypeptide chain further comprises a second Fc region linked to C_{H1}, the second Fc region comprising an immunoglobulin hinge region and C_{H2} and C_{H3} immunoglobulin heavy chain constant domains; wherein the first and second Fc regions are human IgG1 Fc regions; and wherein the first and the second Fc regions each comprise amino acid substitutions at positions corresponding to positions 234 and 235 of human IgG1 according to EU Index, wherein the amino acid substitutions are L234A and L235A. In some embodiments, the Fc regions of the second and the third polypeptide chains are human IgG1 Fc regions, and wherein the Fc regions each comprise amino acid substitutions at positions corresponding to positions 234 and 235 of human IgG1 according to EU Index, wherein the amino acid substitutions are L234A and L235A. In some embodiments, the second polypeptide chain further comprises a first Fc region linked to C_{H1}, the first Fc region comprising an immunoglobulin hinge region and C_{H2} and C_{H3} immunoglobulin heavy chain constant domains; wherein the third polypeptide chain further comprises a second Fc region linked to C_{H1}, the second Fc region comprising an immunoglobulin hinge region and C_{H2} and C_{H3} immunoglobulin heavy chain constant domains; wherein the first and second Fc regions are human IgG1 Fc regions; and wherein the first and the second Fc regions each comprise amino acid substitutions at positions corresponding to positions 234, 235, and 329 of human IgG1 according to EU Index, wherein the amino acid substitutions are L234A, L235A, and P329A. In some embodiments, the Fc regions of the second and the third polypeptide chains are human IgG1 Fc regions, and wherein the Fc regions each comprise amino acid substitutions at positions corresponding to positions 234, 235, and 329 of human IgG1 according to EU Index, wherein the amino acid substitutions are L234A, L235A, and P329A. In some embodiments, the Fc regions of the second and the third polypeptide chains are human IgG4 Fc regions, and the Fc regions each comprise amino acid substitutions at positions corresponding to positions 234 and 235 of human IgG4 according to EU Index, wherein the amino acid substitutions are F234A and L235A. In some embodiments, the binding protein comprises a second polypeptide chain further comprising a first Fc region linked to C_{H1}, the first Fc region comprising an immunoglobulin hinge region and C_{H2} and C_{H3} immunoglobulin heavy chain constant domains, and a third polypeptide chain further comprising a second Fc region linked to C_{H1}, the second Fc region comprising an immunoglobulin hinge region and C_{H2} and C_{H3} immunoglobulin heavy chain constant domains; and wherein the first and the second Fc regions each comprise amino acid substitutions at positions corresponding to positions 234 and 235 of human IgG4 according to EU Index, wherein the amino acid substitutions are F234A and L235A.

In some embodiments, the second polypeptide chain further comprises a first Fc region linked to CH1, wherein the first Fc region is a human IgG4 Fc region comprising an immunoglobulin hinge region and C_{H2} and C_{H3} immunoglobulin heavy chain constant domains, wherein the first Fc region comprises amino acid substitutions at positions corresponding to positions 228, 234, 235, 354, 366, and 409 of human IgG4 according to EU Index, wherein the amino acid substitutions are S228P, F234A, L235A, S354C, T366W, and R409K; and wherein the third polypeptide chain further comprises a second Fc region linked to CH1, wherein the second Fc region is a human IgG4 Fc region comprising an immunoglobulin hinge region and C_{H2} and C_{H3} immunoglobulin heavy chain constant domains, wherein the second Fc region comprises amino acid substitutions at positions corresponding to positions 228, 234, 235, 349, 366, 368, 407, and 409 of human IgG4 according to EU Index, wherein the amino acid substitutions are S228P, F234A, L235A, Y349C, T366S, L368A, Y407V, and R409K. In some embodiments, the second polypeptide chain further comprises a first Fc region linked to CH1, wherein the first Fc region is a human IgG4 Fc region comprising an immunoglobulin hinge region and C_{H2} and C_{H3} immunoglobulin heavy chain constant domains, wherein the first Fc region comprises amino acid substitutions at positions corresponding to positions 228, 234, 235, 349, 366, 368, 407, and 409 of human IgG4 according to EU Index, wherein the amino acid substitutions are S228P, F234A, L235A, Y349C, T366S, L368A, Y407V, and R409K; and wherein the third polypeptide chain further comprises a second Fc region linked to CH1, wherein the second Fc region is a human IgG4 Fc region comprising an immunoglobulin hinge region and C_{H2} and C_{H3} immunoglobulin heavy chain constant domains, wherein the second Fc region comprises amino acid substitutions at positions corresponding to positions 228, 234, 235, 354, 366, and 409 of human IgG4 according to EU Index, wherein the amino acid substitutions are S228P, F234A, L235A, S354C, T366W, and R409K.

In some embodiments, the Fc region is a human IgG4 Fc region comprising one or more mutations that reduce or eliminate FcγI and/or FcγII binding. In some embodiments, the Fc region is a human IgG4 Fc region comprising one or more mutations that reduce or eliminate FcγI and/or FcγII binding but do not affect FcRn binding. In some embodiments, the Fc region is a human IgG4 Fc region comprising amino acid substitutions at positions corresponding to positions 228 and/or 409 of human IgG4 according to EU Index. In some embodiments, the amino acid substitutions are S228P and/or R409K. In some embodiments, the Fc region is a human IgG4 Fc region comprising amino acid substitutions at positions corresponding to positions 234 and/or 235 of human IgG4 according to EU Index. In some embodiments, the amino acid substitutions are F234A and/or L235A. In some embodiments, the Fc region is a human IgG4 Fc region comprising amino acid substitutions at positions corresponding to positions 228, 234, 235, and/or 409 of human IgG4 according to EU Index. In some embodiments, the amino acid substitutions are S228P, F234A, L235A, and/or R409K. In some embodiments, the Fc region is a human IgG4 Fc region comprising amino acid substitutions at positions corresponding to positions 233-236 of human IgG4 according to EU Index. In some embodiments, the amino acid substitutions are E233P, F234V, L235A, and a deletion at 236. In some embodiments, the Fc region is a human IgG4 Fc region comprising amino acid mutations at substitutions corresponding to positions 228, 233-236, and/or 409 of human IgG4 according to EU Index. In some embodiments, the amino acid mutations are S228P; E233P, F234V, L235A, and a deletion at 236; and/or R409K.

In some embodiments, the Fc region comprises one or more mutations that reduce or eliminate Fc receptor binding and/or effector function of the Fc region (e.g., Fc receptor-mediated antibody-dependent cellular phagocytosis (ADCP), complement-dependent cytotoxicity (CDC), and/or antibody-dependent cellular cytotoxicity (ADCC)).

In some embodiments, the Fc region is a human IgG1 Fc region comprising one or more amino acid substitutions at positions corresponding to positions 234, 235, and/or 329 of human IgG1 according to EU Index. In some embodiments, the amino acid substitutions are L234A, L235A, and/or P329A. In some embodiments, the Fc region is a human IgG1 Fc region comprising amino acid substitutions at positions corresponding to positions 298, 299, and/or 300 of human IgG1 according to EU Index. In some embodiments, the amino acid substitutions are S298N, T299A, and/or Y300S. In some embodiments, the Fc region is a human IgG1 Fc region comprising one or more amino acid substitutions at positions corresponding to positions 256 and/or 307 of human IgG1 according to EU Index. In some embodiments, the amino acid substitutions are T256D and/or T307Q.

In some embodiments, a binding protein of the present disclosure comprises one or more mutations to improve purification, e.g., by modulating the affinity for a purincation reagent. For example, it is known that heterodimeric binding proteins can be selectively purified away from their homodimeric forms if one of the two Fc regions of the heterodimeric form contains mutation(s) that reduce or eliminate binding to Protein A, because the heterodimeric form will have an intermediate affinity for Protein A-based purification than either homodimeric form and can be selectively eluted from Protein A, e.g., by use of a different pH (See e.g., Smith, E. J. et al. (2015) Sci. Rep. 5:17943). In some embodiments, the mutation comprises substitutions at positions corresponding to positions 435 and 436 of human IgG1 or IgG4 according to EU Index, wherein the amino acid substitutions are H435R and Y436F. In some embodiments, the binding protein comprises a second polypeptide chain further comprising a first Fc region linked to C_{H1}, the first Fc region comprising an immunoglobulin hinge region and C_{H2} and C_{H3} immunoglobulin heavy chain constant domains, and a third polypeptide chain further comprising a second Fc region linked to C_{H1}, the second Fc region comprising an immunoglobulin hinge region and C_{H2} and C_{H3} immunoglobulin heavy chain constant domains; and wherein only one of the first and the second Fc regions comprises amino acid substitutions at positions corresponding to positions 435 and 436 of human IgG1 or IgG4 according to EU Index, wherein the amino acid substitutions are H435R and Y436F. In some embodiments, a binding protein of the present disclosure comprises knob and hole mutations and one or more mutations to improve puriication. In some embodiments, the first and/or second Fc regions are human IgG1 Fc regions. In some embodiments, the first and/or second Fc regions are human IgG4 Fc regions.

In some embodiments, a binding protein of the present disclosure comprises one or more mutations to improve stability, e.g., of the hinge region and/or dimer interface of IgG4 (See e.g., Spiess, C. et al. (2013) J Biol. Chem. 288:26583-26593). In some embodiments, the mutation comprises substitutions at positions corresponding to positions 228 and 409 of human IgG4 according to EU Index, wherein the amino acid substitutions are S228P and R409K. In some embodiments, the binding protein comprises a second polypeptide chain further comprising a first Fc region linked to C_{H1}, the first Fc region comprising an immunoglobulin hinge region and C_{H2} and C_{H3} immunoglobulin heavy chain constant domains, and a third polypeptide chain further comprising a second Fc region linked to C_{H1}, the second Fc region comprising an immunoglobulin hinge region and C_{H2} and C_{H3} immunoglobulin heavy chain constant domains; wherein the first and second Fc regions are human IgG4 Fc regions; and wherein the first and the second Fc regions each comprise amino acid substitutions at positions corresponding to positions 228 and 409 of human IgG4 according to EU Index, wherein the amino acid substitutions are S228P and R409K. In some embodiments, a binding protein of the present disclosure comprises knob and hole mutations and one or more mutations to improve stability. In some embodiments, the first and/or second Fc regions are human IgG4 Fc regions.

In some embodiments, the Fc region is a human IgG1 Fc region comprising one or more amino acid substitutions at positions corresponding to positions 234, 235, and/or 329 of human IgG1 according to EU Index. In some embodiments, the amino acid substitutions are L234A, L235A, and/or P329A. In some embodiments, the Fc region is a human IgG1 Fc region comprising amino acid substitutions at positions corresponding to positions 298, 299, and/or 300 of human IgG1 according to EU Index. In some embodiments, the amino acid substitutions are S298N, T299A, and/or Y300S.

### Nucleic Acids

Other aspects of the present disclosure relate to isolated nucleic acid molecules comprising a nucleotide sequence encoding any of the binding proteins described herein, including monospecific/monovalent, bispecific/bivalent, trispecific/trivalent, or multispecific/multivalent binding proteins, e.g., as described supra.

Other aspects of the present disclosure relate to kits of polynucleotides, e.g., that encode one or more polypeptides of a binding protein as described herein. For example, in some embodiments, the kits comprise a first polynucleotide that encodes a first polypeptide of a trispecific binding protein of the present disclosure, a second polynucleotide that encodes a second polypeptide of a trispecific binding protein of the present disclosure, a third polynucleotide that encodes a third polypeptide of a trispecific binding protein of the present disclosure, and a fourth polynucleotide that encodes a fourth polypeptide of a trispecific binding protein of the present disclosure. In some embodiments, the kits comprise a first polynucleotide that encodes a heavy chain polypeptide of an antibody of the present disclosure, and a second polynucleotide that encodes a light chain polypeptide of an antibody of the present disclosure.

Other aspects of the present disclosure relate to a vector system comprising one or more vectors encoding a first, second, third, and fourth polypeptide chain of any of the binding proteins described herein. In some embodiments, the vector system comprises a first vector encoding the first polypeptide chain of the binding protein, a second vector encoding the second polypeptide chain of the binding protein, a third vector encoding the third polypeptide chain of the binding protein, and a fourth vector encoding the fourth polypeptide chain of the binding protein. In some embodiments, the vector system comprises a first vector that encodes a heavy chain polypeptide of an antibody of the present disclosure, and a second vector that encodes a light chain polypeptide of an antibody of the present disclosure.

Standard recombinant DNA methodologies are used to construct the polynucleotides that encode the polypeptides which form the binding proteins, incorporate these polynucleotides into recombinant expression vectors, and introduce such vectors into host cells. See e.g., Sambrook et al., 2001, MOLECULAR CLONING: A LABORATORY MANUAL (Cold Spring Harbor Laboratory Press, 3rd ed.). Enzymatic reactions and purification techniques may be performed according to manufacturer's specifications, as commonly accomplished in the art, or as described herein. Unless specific definitions are provided, the nomenclature utilized in connection with, and the laboratory procedures and techniques of, analytical chemistry, synthetic organic chemistry, and medicinal and pharmaceutical chemistry described herein are those well-known and commonly used in the art. Similarly, conventional techniques may be used for chemical syntheses, chemical analyses, pharmaceutical preparation, formulation, delivery, and treatment of patients.

In some embodiments, the isolated nucleic acid is operably linked to a heterologous promoter to direct transcription of the binding protein-coding nucleic acid sequence. A promoter may refer to nucleic acid control sequences which direct transcription of a nucleic acid. A first nucleic acid sequence is operably linked to a second nucleic acid sequence when the first nucleic acid sequence is placed in a functional relationship with the second nucleic acid sequence. For instance, a promoter is operably linked to a coding sequence of a binding protein if the promoter affects the transcription or expression of the coding sequence. Examples of promoters may include, but are not limited to, promoters obtained from the genomes of viruses (such as polyoma virus, fowlpox virus, adenovirus (such as Adenovirus 2), bovine papilloma virus, avian sarcoma virus, cytomegalovirus, a retrovirus, hepatitis-B virus, Simian Virus 40 (SV40), and the like), from heterologous eukaryotic promoters (such as the actin promoter, an immunoglobulin promoter, from heat-shock promoters, and the like), the CAG-promoter (Niwa et al., Gene 108(2):193-9, 1991), the phosphoglycerate kinase (PGK)-promoter, a tetracycline-inducible promoter (Masui et al., Nucleic Acids Res. 33:e43, 2005), the lac system, the trp system, the tac system, the trc system, major operator and promoter regions of phage lambda, the promoter for 3-phosphoglycerate kinase, the promoters of yeast acid phosphatase, and the promoter of the yeast alpha-mating factors. Polynucleotides encoding binding proteins of the present disclosure may be under the control of a constitutive promoter, an inducible promoter, or any other suitable promoter described herein or other suitable promoter that will be readily recognized by one skilled in the art.

In some embodiments, the protein(s) to be expressed (e.g., a polypeptide belonging to an antibody or binding protein of the present disclosure) comprise a leader sequence that allows the protein to be secreted from a cell and is cleaved afterwards. An exemplary leader peptide sequence is MGWSCIILFLVATATGVHS (SEQ ID NO:107).

In some embodiments, the isolated nucleic acid is incorporated into a vector. In some embodiments, the vector is an expression vector. Expression vectors may include one or more regulatory sequences operatively linked to the polynucleotide to be expressed. The term “regulatory sequence” includes promoters, enhancers and other expression control elements (e.g., polyadenylation signals). Examples of suitable enhancers may include, but are not limited to, enhancer sequences from mammalian genes (such as globin, elastase, albumin, a-fetoprotein, insulin and the like), and enhancer sequences from a eukaryotic cell virus (such as SV40 enhancer on the late side of the replication origin (bp 100-270), the cytomegalovirus early promoter enhancer, the polyoma enhancer on the late side of the replication origin, adenovirus enhancers, and the like). Examples of suitable vectors may include, for example, plasmids, cosmids, episomes, transposons, and viral vectors (e.g., adenoviral, vaccinia viral, Sindbis-viral, measles, herpes viral, lentiviral, retroviral, adeno-associated viral vectors, etc.). Expression vectors can be used to transfect host cells, such as, for example, bacterial cells, yeast cells, insect cells, and mammalian cells. Biologically functional viral and plasmid DNA vectors capable of expression and replication in a host are known in the art, and can be used to transfect any cell of interest.

### Host Cells

Other aspects of the present disclosure relate to a host cell (e.g., an isolated host cell) comprising one or more isolated polynucleotides, vectors, and/or vector systems described herein. In some embodiments, an isolated host cell of the present disclosure is cultured in vitro. In some embodiments, the host cell is a bacterial cell (e.g., an E. coli cell). In some embodiments, the host cell is a yeast cell (e.g., an S. cerevisiae cell). In some embodiments, the host cell is an insect cell. Examples of insect host cells may include, for example, Drosophila cells (e.g., S2 cells), Trichoplusia ni cells (e.g., High Five™ cells), and Spodopterafrugiperda cells (e.g., Sf21 or Sf9 cells). In some embodiments, the host cell is a mammalian cell. Examples of mammalian host cells may include, for example, human embryonic kidney cells (e.g., 293 or 293 cells subcloned for growth in suspension culture), Expi293™ cells, CHO cells, baby hamster kidney cells (e.g., BHK, ATCC CCL 10), mouse sertoli cells (e.g., TM4 cells), monkey kidney cells (e.g., CV1 ATCC CCL 70), African green monkey kidney cells (e.g., VERO-76, ATCC CRL-1587), human cervical carcinoma cells (e.g., HELA, ATCC CCL 2), canine kidney cells (e.g., MDCK, ATCC CCL 34), buffalo rat liver cells (e.g., BRL 3A, ATCC CRL 1442), human lung cells (e.g., W138, ATCC CCL 75), human liver cells (e.g., Hep G2, HB 8065), mouse mammary tumor cells (e.g., MMT 060562, ATCC CCL51), TRI cells, MRC 5 cells, FS4 cells, a human hepatoma line (e.g., Hep G2), and myeloma cells (e.g., NSO and Sp2/0 cells).

Other aspects of the present disclosure relate to a method of producing any of the binding proteins described herein. In some embodiments, the method includes a) culturing a host cell (e.g., any of the host cells described herein) comprising an isolated nucleic acid, vector, and/or vector system (e.g., any of the isolated nucleic acids, vectors, and/or vector systems described herein) under conditions such that the host cell expresses the binding protein; and b) isolating the binding protein from the host cell. Methods of culturing host cells under conditions to express a protein are well known to one of ordinary skill in the art. Methods of isolating proteins from cultured host cells are well known to one of ordinary skill in the art, including, for example, by affinity chromatography (e.g., two step affinity chromatography comprising protein A affinity chromatography followed by size exclusion chromatography).

### Pharmaceutical Compositions

Therapeutic or pharmaceutical compositions comprising binding proteins are within the scope of the disclosure. Such therapeutic or pharmaceutical compositions can comprise a therapeutically effective amount of a binding protein, or binding protein-drug conjugate, in admixture with a pharmaceutically or physiologically acceptable formulation agent selected for suitability with the mode of administration.

Acceptable formulation materials are nontoxic to recipients at the dosages and concentrations employed.

The pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides—e.g., sodium or potassium chloride—or mannitol sorbitol), delivery vehicles, diluents, excipients and/or pharmaceutical adjuvants (see, e.g., REMINGTON'S PHARMACEUTICAL SCIENCES (18th Ed., A.R. Gennaro, ed., Mack Publishing Company 1990), and subsequent editions of the same, incorporated herein by reference for any purpose).

The optimal pharmaceutical composition will be determined by a skilled artisan depending upon, for example, the intended route of administration, delivery format, and desired dosage. Such compositions can influence the physical state, stability, rate of in vivo release, and rate of in vivo clearance of the binding protein.

The primary vehicle or carrier in a pharmaceutical composition can be either aqueous or non-aqueous in nature. For example, a suitable vehicle or carrier for injection can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which can further include sorbitol or a suitable substitute. In one embodiment of the disclosure, binding protein compositions can be prepared for storage by mixing the selected composition having the desired degree of purity with optional formulation agents in the form of a lyophilized cake or an aqueous solution. Further, the binding protein can be formulated as a lyophilizate using appropriate excipients such as sucrose.

The pharmaceutical compositions of the disclosure can be selected for parenteral delivery or subcutaneous. Alternatively, the compositions can be selected for inhalation or for delivery through the digestive tract, such as orally. The preparation of such pharmaceutically acceptable compositions is within the skill of the art.

The formulation components are present in concentrations that are acceptable to the site of administration. For example, buffers are used to maintain the composition at physiological pH or at a slightly lower pH, typically within a pH range of from about 5 to about 8.

When parenteral administration is contemplated, the therapeutic compositions for use can be in the form of a pyrogen-free, parenterally acceptable, aqueous solution comprising the desired binding protein in a pharmaceutically acceptable vehicle. A particularly suitable vehicle for parenteral injection is sterile distilled water in which a binding protein is formulated as a sterile, isotonic solution, properly preserved. Yet another preparation can involve the formulation of the desired molecule with an agent, such as injectable microspheres, bio-erodible particles, polymeric compounds (such as polylactic acid or polyglycolic acid), beads, or liposomes, that provides for the controlled or sustained release of the product which can then be delivered via a depot injection. Hyaluronic acid can also be used, and this can have the effect of promoting sustained duration in the circulation. Other suitable means for the introduction of the desired molecule include implantable drug delivery devices.

In one embodiment, a pharmaceutical composition can be formulated for inhalation. For example, a binding protein can be formulated as a dry powder for inhalation. Binding protein inhalation solutions can also be formulated with a propellant for aerosol delivery. In yet another embodiment, solutions can be nebulized.

It is also contemplated that certain formulations can be administered orally. In one embodiment of the disclosure, binding proteins that are administered in this fashion can be formulated with or without those carriers customarily used in the compounding of solid dosage forms such as tablets and capsules. For example, a capsule can be designed to release the active portion of the formulation at the point in the gastrointestinal tract where bioavailability is maximized and pre-systemic degradation is minimized. Additional agents can be included to facilitate absorption of the binding protein. Diluents, flavorings, low melting point waxes, vegetable oils, lubricants, suspending agents, tablet disintegrating agents, and binders can also be employed.

Another pharmaceutical composition can involve an effective quantity of binding proteins in a mixture with non-toxic excipients that are suitable for the manufacture of tablets. By dissolving the tablets in sterile water, or another appropriate vehicle, solutions can be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions of the disclosure will be evident to those skilled in the art, including formulations involving binding proteins in sustained- or controlled-delivery formulations. Techniques for formulating a variety of other sustained- or controlled-delivery means, such as liposome carriers, bio-erodible microparticles or porous beads and depot injections, are also known to those skilled in the art. Additional examples of sustained-release preparations include semipermeable polymer matrices in the form of shaped articles, e.g. films, or microcapsules. Sustained release matrices can include polyesters, hydrogels, polylactides, copolymers of L-glutamic acid and gamma ethyl-L-glutamate, poly(2-hydroxyethyl-methacrylate), ethylene vinyl acetate, or poly-D(-)-3-hydroxybutyric acid. Sustained-release compositions can also include liposomes, which can be prepared by any of several methods known in the art.

Pharmaceutical compositions to be used for in vivo administration typically must be sterile. This can be accomplished by filtration through sterile filtration membranes. Where the composition is lyophilized, sterilization using this method can be conducted either prior to, or following, lyophilization and reconstitution. The composition for parenteral administration can be stored in lyophilized form or in a solution. In addition, parenteral compositions generally are placed into a container having a sterile access port, for example, an intravenous solution bag or vial having a stopper pierceable by a hypodermic injection needle.

Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) requiring reconstitution prior to administration.

The disclosure also encompasses kits for producing a single-dose administration unit. The kits can each contain both a first container having a dried protein and a second container having an aqueous formulation. Also included within the scope of this disclosure are kits containing single and multi-chambered pre-filled syringes (e.g., liquid syringes and lyosyringes).

The effective amount of a binding protein pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the binding protein is being used, the route of administration, and the size (body weight, body surface, or organ size) and condition (the age and general health) of the patient. Accordingly, the clinician can titer the dosage and modify the route of administration to obtain the optimal therapeutic effect.

Dosing frequency will depend upon the pharmacokinetic parameters of the binding protein in the formulation being used. Typically, a clinician will administer the composition until a dosage is reached that achieves the desired effect. The composition can therefore be administered as a single dose, as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages can be ascertained through use of appropriate dose-response data.

The route of administration of the pharmaceutical composition is in accord with known methods, e.g., orally; through injection by subcutaneous, intravenous, intraperitoneal, intracerebral (intraparenchymal), intracerebroventricular, intramuscular, intraocular, intraarterial, intraportal, or intralesional routes; by sustained release systems; or by implantation devices. Where desired, the compositions can be administered by bolus injection or continuously by infusion, or by implantation device.

The composition can also be administered locally via implantation of a membrane, sponge, or other appropriate material onto which the desired molecule has been absorbed or encapsulated. Where an implantation device is used, the device can be implanted into any suitable tissue or organ, and delivery of the desired molecule can be via diffusion, timed-release bolus, or continuous administration.

The disclosure also relates to a kit comprising a binding protein and other reagents useful for detecting target antigen levels in biological samples. Such reagents can include a detectable label, blocking serum, positive and negative control samples, and detection reagents. In some embodiments, the kit comprises a composition comprising any binding protein, polynucleotide, vector, vector system, and/or host cell described herein. In some embodiments, the kit comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing a condition and may have a sterile access port (for example the container may be an intravenous solution bag or a vial having a stopper pierceable by a hypodermic injection needle). In some embodiments, the label or package insert indicates that the composition is used for preventing, diagnosing, and/or treating the condition of choice. Alternatively, or additionally, the article of manufacture or kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Methods and Uses for Binding Proteins

Certain aspects of the present disclosure relate to methods for preventing and/or treating and/or alleviating one or more symptoms of an inflammatory disease or disorder (e.g., atopic dermatitis) in a patient, e.g., in need thereof. In some embodiments, the methods comprise administering to the patient a therapeutically effective amount of a binding protein or pharmaceutical composition of the present disclosure. In some embodiments, the patient is a human.

Also provided herein is a binding protein or pharmaceutical composition of the present disclosure for use in preventing and/or treating and/or alleviating one or more symptoms of an inflammatory disease or disorder (e.g., atopic dermatitis) in a patient. In some embodiments, the binding protein or pharmaceutical composition for its uses comprises administering to the patient a therapeutically effective amount of a binding protein or pharmaceutical composition of the present disclosure. In some embodiments, the patient is a human.

Also provided herein is use of a binding protein or pharmaceutical composition of the present disclosure in the manufacture of a medicament for preventing and/or treating and/or alleviating one or more symptoms of an inflammatory disease or disorder (e.g., atopic dermatitis) in a patient. In some embodiments, the patient is a human.

Any of the binding proteins described herein may find use in the methods or uses of the present disclosure.

The binding proteins can be employed in any known assay method, such as competitive binding assays, direct and indirect sandwich assays, and immunoprecipitation assays for the detection and quantitation of one or more target antigens. The binding proteins will bind the one or more target antigens with an affinity that is appropriate for the assay method being employed.

For diagnostic applications, in certain embodiments, binding proteins can be labeled with a detectable moiety. The detectable moiety can be any one that is capable of producing, either directly or indirectly, a detectable signal. For example, the detectable moiety can be a radioisotope, such as ³H, ¹⁴C, ³²P ³⁵S, ¹²⁵I, ⁹⁹Tc, ¹¹¹In, or ⁶⁷Ga; a fluorescent or chemiluminescent compound, such as fluorescein isothiocyanate, rhodamine, or luciferin; or an enzyme, such as alkaline phosphatase, B-galactosidase, or horseradish peroxidase.

The binding proteins are also useful for in vivo imaging. A binding protein labeled with a detectable moiety can be administered to an animal, preferably into the bloodstream, and the presence and location of the labeled antibody in the host assayed. The binding protein can be labeled with any moiety that is detectable in an animal, whether by nuclear magnetic resonance, radiology, or other detection means known in the art.

For clinical or research applications, in certain embodiments, binding proteins can be conjugated to a cytotoxic agent. A variety of antibodies coupled to cytotoxic agents (i.e., antibody-drug conjugates) have been used to target cytotoxic payloads to specific tumor cells. Cytotoxic agents and linkers that conjugate the agents to an antibody are known in the art; see, e.g., Parslow, A. C. et al. (2016) Biomedicines 4:14 and Kalim, M. et al. (2017) Drug Des. Devel. Ther. 11:2265-2276.

Therapeutic or pharmaceutical compositions comprising binding proteins are within the scope of the disclosure. Such therapeutic or pharmaceutical compositions can comprise a therapeutically effective amount of a binding protein, or binding protein-drug conjugate, in admixture with a pharmaceutically or physiologically acceptable formulation agent selected for suitability with the mode of administration. These pharmaceutical compositions may find use in any of the methods and uses described herein (e.g., ex vivo, in vitro, and/or in vivo).

Acceptable formulation materials preferably are nontoxic to recipients at the dosages and concentrations employed.

The pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition.

The optimal pharmaceutical composition will be determined by a skilled artisan depending upon, for example, the intended route of administration, delivery format, and desired dosage. Such compositions can influence the physical state, stability, rate of in vivo release, and rate of in vivo clearance of the binding protein.

The primary vehicle or carrier in a pharmaceutical composition can be either aqueous or non-aqueous in nature. For example, a suitable vehicle or carrier for injection can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which can further include sorbitol or a suitable substitute. In one embodiment of the disclosure, binding protein compositions can be prepared for storage by mixing the selected composition having the desired degree of purity with optional formulation agents in the form of a lyophilized cake or an aqueous solution. Further, the binding protein can be formulated as a lyophilizate using appropriate excipients such as sucrose.

The pharmaceutical compositions of the disclosure can be selected for parenteral delivery or subcutaneous. Alternatively, the compositions can be selected for inhalation or for delivery through the digestive tract, such as orally. The preparation of such pharmaceutically acceptable compositions is within the skill of the art.

The formulation components are present in concentrations that are acceptable to the site of administration. For example, buffers are used to maintain the composition at physiological pH or at a slightly lower pH, typically within a pH range of from about 5 to about 8.

When parenteral administration is contemplated, the therapeutic compositions for use can be in the form of a pyrogen-free, parenterally acceptable, aqueous solution comprising the desired binding protein in a pharmaceutically acceptable vehicle. A particularly suitable vehicle for parenteral injection is sterile distilled water in which a binding protein is formulated as a sterile, isotonic solution, properly preserved. Yet another preparation can involve the formulation of the desired molecule with an agent, such as injectable microspheres, bio-erodible particles, polymeric compounds (such as polylactic acid or polyglycolic acid), beads, or liposomes, that provides for the controlled or sustained release of the product which can then be delivered via a depot injection. Hyaluronic acid can also be used, and this can have the effect of promoting sustained duration in the circulation. Other suitable means for the introduction of the desired molecule include implantable drug delivery devices.

In one embodiment, a pharmaceutical composition can be formulated for inhalation. For example, a binding protein can be formulated as a dry powder for inhalation. Binding protein inhalation solutions can also be formulated with a propellant for aerosol delivery. In yet another embodiment, solutions can be nebulized.

It is also contemplated that certain formulations can be administered orally. In one embodiment of the disclosure, binding proteins that are administered in this fashion can be formulated with or without those carriers customarily used in the compounding of solid dosage forms such as tablets and capsules. For example, a capsule can be designed to release the active portion of the formulation at the point in the gastrointestinal tract when bioavailability is maximized and pre-systemic degradation is minimized. Additional agents can be included to facilitate absorption of the binding protein. Diluents, flavorings, low melting point waxes, vegetable oils, lubricants, suspending agents, tablet disintegrating agents, and binders can also be employed.

Another pharmaceutical composition can involve an effective quantity of binding proteins in a mixture with non-toxic excipients that are suitable for the manufacture of tablets. By dissolving the tablets in sterile water, or another appropriate vehicle, solutions can be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions of the disclosure will be evident to those skilled in the art, including formulations involving binding proteins in sustained- or controlled-delivery formulations. Techniques for formulating a variety of other sustained- or controlled-delivery means, such as liposome carriers, bio-erodible microparticles or porous beads and depot injections, are also known to those skilled in the art. Additional examples of sustained-release preparations include semipermeable polymer matrices in the form of shaped articles, e.g. films, or microcapsules. Sustained release matrices can include polyesters, hydrogels, polylactides, copolymers of L-glutamic acid and gamma ethyl-L-glutamate, poly(2-hydroxyethyl-methacrylate), ethylene vinyl acetate, or poly-D(-)-3-hydroxybutyric acid. Sustained-release compositions can also include liposomes, which can be prepared by any of several methods known in the art.

Pharmaceutical compositions to be used for in vivo administration typically must be sterile. This can be accomplished by filtration through sterile filtration membranes. Where the composition is lyophilized, sterilization using this method can be conducted either prior to, or following, lyophilization and reconstitution. The composition for parenteral administration can be stored in lyophilized form or in a solution. In addition, parenteral compositions generally are placed into a container having a sterile access port, for example, an intravenous solution bag or vial having a stopper pierceable by a hypodermic injection needle.

Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) requiring reconstitution prior to administration.

The disclosure also relates to a kit comprising a binding protein and other reagents useful for detecting target antigen levels in biological samples. Such reagents can include a detectable label, blocking serum, positive and negative control samples, and detection reagents. In some embodiments, the kit comprises a composition comprising any binding protein, polynucleotide, vector, vector system, and/or host cell described herein. In some embodiments, the kit comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing a condition and may have a sterile access port (for example the container may be an intravenous solution bag or a vial having a stopper pierceable by a hypodermic injection needle). In some embodiments, the label or package insert indicates that the composition is used for preventing, diagnosing, and/or treating the condition of choice. Alternatively, or additionally, the article of manufacture or kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The disclosure also encompasses kits for producing a single-dose administration unit. The kits can each contain both a first container having a dried protein and a second container having an aqueous formulation. Also included within the scope of this disclosure are kits containing single and multi-chambered pre-filled syringes (e.g., liquid syringes and lyosyringes).

The effective amount of a binding protein pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the binding protein is being used, the route of administration, and the size (body weight, body surface, or organ size) and condition (the age and general health) of the patient. Accordingly, the clinician can titer the dosage and modify the route of administration to obtain the optimal therapeutic effect.

Dosing frequency will depend upon the pharmacokinetic parameters of the binding protein in the formulation being used. Typically, a clinician will administer the composition until a dosage is reached that achieves the desired effect. The composition can therefore be administered as a single dose, as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages can be ascertained through use of appropriate dose-response data.

The route of administration of the pharmaceutical composition is in accord with known methods, e.g., orally; through injection by intravenous, intraperitoneal, intracerebral (intraparenchymal), intracerebroventricular, intramuscular, intraocular, intraarterial, intraportal, or intralesional routes; by sustained release systems; or by implantation devices. Where desired, the compositions can be administered by bolus injection or continuously by infusion, or by implantation device.

The composition can also be administered locally via implantation of a membrane, sponge, or other appropriate material onto which the desired molecule has been absorbed or encapsulated. Where an implantation device is used, the device can be implanted into any suitable tissue or organ, and delivery of the desired molecule can be via diffusion, timed-release bolus, or continuous administration.

### Exemplary Embodiments

The following exemplary embodiments are representative of some aspects of the invention: Embodiment 1. A trispecific binding protein comprising four polypeptide chains that form three antigen binding sites, wherein a first polypeptide chain of the binding protein comprises a structure represented by the formula:

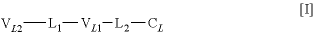

and a second polypeptide chain of the binding protein comprises a structure represented by the formula:

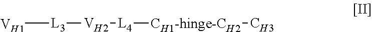

and a third polypeptide chain of the binding protein comprises a structure represented by the formula:

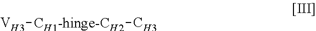

and a fourth polypeptide chain of the binding protein comprises a structure represented by the formula:

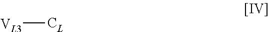

wherein:
   V_{L1} is a first immunoglobulin light chain variable domain;
   V_{L2} is a second immunoglobulin light chain variable domain;
   V_{L3} is a third immunoglobulin light chain variable domain;
   V_{H1} is a first immunoglobulin heavy chain variable domain;
   V_{H2} is a second immunoglobulin heavy chain variable domain;
   V_{H3} is a third immunoglobulin heavy chain variable domain;
   C_{L} is an immunoglobulin light chain constant domain;
   C_{H1} is an immunoglobulin C_{H1} heavy chain constant domain;
   C_{H2} is an immunoglobulin C_{H2} heavy chain constant domain;
   C_{H3} is an immunoglobulin C_{H3} heavy chain constant domain;
   hinge is an immunoglobulin hinge region connecting the C_{H1} and C_{H2} domains; and
   L₁, L₂, L₃ and L₄ are each independently amino acid linkers or zero amino acids in length;
wherein the polypeptide of formula I and the polypeptide of formula II form a cross-over light chain-heavy chain pair of the binding protein; and
wherein V_{H1} and V_{L1} form a binding pair and a first antigen binding site, V_{H2} and V_{L2} form a binding pair and a second antigen binding site, and V_{H3} and V_{L3} form a binding pair and a third antigen binding site;
wherein the first antigen binding site specifically binds an interleukin-4 (IL-4) polypeptide and the second antigen binding site specifically binds an interleukin-13 (IL-13) polypeptide, or the first antigen binding site specifically binds an IL-13 polypeptide and the second antigen binding site specifically binds an IL-4 polypeptide;
wherein the binding site that specifically binds an IL-4 polypeptide comprises:
   (a) a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence selected from the group consisting of SEQ ID NOs:3-5, and a VH domain that comprises a CDR-L1 comprising the amino acid sequence of SEQ ID NO:6 or 7, a CDR-L2 comprising the amino acid sequence of SEQ ID NO:8 or 9, and a CDR-L3 comprising the amino acid sequence of SEQ ID NO:10 or 11, or
   (b) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence selected from the group consisting of SEQ ID NOs:24-27, and a VH domain that comprises a CDR-L1 comprising the amino acid sequence of SEQ ID NO:6 or 7, a CDR-L2 comprising the amino acid sequence of SEQ ID NO:8 or 9, and a CDR-L3 comprising the amino acid sequence of SEQ ID NO:10 or 11; and
wherein the binding site that specifically binds an IL-13 polypeptide comprises a VH domain that comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59 or 108, and a VH domain that comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62.
   Embodiment 2. The binding protein of embodiment 1, wherein the first binding site specifically binds the IL-4 polypeptide, and the second binding site specifically binds the IL-13 polypeptide.
   Embodiment 3. The binding protein of embodiment 1 or embodiment 2, wherein the binding site that specifically binds an IL-4 polypeptide comprises:
(a) a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3, and a VH domain that comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10;
(b) a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11;
(c) a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:4, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11; or
(d) a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:5, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11.
   Embodiment 4. The binding protein of embodiment 3, wherein the binding site that specifically binds an IL-4 polypeptide comprises:
(a) a VH domain that comprises an amino acid sequence that is at least 95%, at least 96%, at least 97%, at least 98%, at least 99%, or 100% identical to the amino acid sequence of SEQ ID NO:34 and a VH domain that comprises an amino acid sequence that is at least 95%, at least 96%, at least 97%, at least 98%, at least 99%, or 100% identical to the amino acid sequence of SEQ ID NO:35;
(b) a VH domain that comprises an amino acid sequence that is at least 95%, at least 96%, at least 97%, at least 98%, at least 99%, or 100% identical to the amino acid sequence of SEQ ID NO:36 and a VH domain that comprises an amino acid sequence that is at least 95%, at least 96%, at least 97%, at least 98%, at least 99%, or 100% identical to the amino acid sequence of SEQ ID NO:37;
(c) a VH domain that comprises an amino acid sequence that is at least 95%, at least 96%, at least 97%, at least 98%, at least 99%, or 100% identical to the amino acid sequence of SEQ ID NO:38 and a VH domain that comprises an amino acid sequence that is at least 95%, at least 96%, at least 97%, at least 98%, at least 99%, or 100% identical to the amino acid sequence of SEQ ID NO:39;
(d) a VH domain that comprises an amino acid sequence that is at least 95%, at least 96%, at least 97%, at least 98%, at least 99%, or 100% identical to the amino acid sequence of SEQ ID NO:40 and a VH domain that comprises an amino acid sequence that is at least 95%, at least 96%, at least 97%, at least 98%, at least 99%, or 100% identical to the amino acid sequence of SEQ ID NO:41;
(e) a VH domain that comprises an amino acid sequence that is at least 95%, at least 96%, at least 97%, at least 98%, at least 99%, or 100% identical to the amino acid sequence of SEQ ID NO:42 and a VH domain that comprises an amino acid sequence that is at least 95%, at least 96%, at least 97%, at least 98%, at least 99%, or 100% identical to the amino acid sequence of SEQ ID NO:43;
(f) a VH domain that comprises an amino acid sequence that is at least 95%, at least 96%, at least 97%, at least 98%, at least 99%, or 100% identical to the amino acid sequence of SEQ ID NO:44 and a VH domain that comprises an amino acid sequence that is at least 95%, at least 96%, at least 97%, at least 98%, at least 99%, or 100% identical to the amino acid sequence of SEQ ID NO:43; or
(g) a VH domain that comprises an amino acid sequence that is at least 95%, at least 96%, at least 97%, at least 98%, at least 99%, or 100% identical to the amino acid sequence of SEQ ID NO:45 and a VH domain that comprises an amino acid sequence that is at least 95%, at least 96%, at least 97%, at least 98%, at least 99%, or 100% identical to the amino acid sequence of SEQ ID NO:43.
   Embodiment 5. The binding protein of embodiment 4, wherein:
(a) V_{H1} comprises an amino acid sequence that is at least 95%, at least 96%, at least 97%, at least 98%, at least 99%, or 100% identical to the amino acid sequence of SEQ ID NO:34, and V_{L1} comprises an amino acid sequence that is at least 95%, at least 96%, at least 97%, at least 98%, at least 99%, or 100% identical to the amino acid sequence of SEQ ID NO:35;
(b) V_{H1} comprises an amino acid sequence that is at least 95%, at least 96%, at least 97%, at least 98%, at least 99%, or 100% identical to the amino acid sequence of SEQ ID NO:36, and V_{L1} comprises an amino acid sequence that is at least 95%, at least 96%, at least 97%, at least 98%, at least 99%, or 100% identical to the amino acid sequence of SEQ ID NO:37;
(c) V_{H1} comprises an amino acid sequence that is at least 95%, at least 96%, at least 97%, at least 98%, at least 99%, or 100% identical to the amino acid sequence of SEQ ID NO:38, and V_{L1} comprises an amino acid sequence that is at least 95%, at least 96%, at least 97%, at least 98%, at least 99%, or 100% identical to the amino acid sequence of SEQ ID NO:39;
(d) V_{H1} comprises an amino acid sequence that is at least 95%, at least 96%, at least 97%, at least 98%, at least 99%, or 100% identical to the amino acid sequence of SEQ ID NO:40, and V_{L1} comprises an amino acid sequence that is at least 95%, at least 96%, at least 97%, at least 98%, at least 99%, or 100% identical to the amino acid sequence of SEQ ID NO:41;
(e) V_{H1} comprises an amino acid sequence that is at least 95%, at least 96%, at least 97%, at least 98%, at least 99%, or 100% identical to the amino acid sequence of SEQ ID NO:42, and V_{L1} comprises an amino acid sequence that is at least 95%, at least 96%, at least 97%, at least 98%, at least 99%, or 100% identical to the amino acid sequence of SEQ ID NO:43;
(f) V_{H1} comprises an amino acid sequence that is at least 95%, at least 96%, at least 97%, at least 98%, at least 99%, or 100% identical to the amino acid sequence of SEQ ID NO:44, and V_{L1} comprises an amino acid sequence that is at least 95%, at least 96%, at least 97%, at least 98%, at least 99%, or 100% identical to the amino acid sequence of SEQ ID NO:43; or
(g) V_{H1} comprises an amino acid sequence that is at least 95%, at least 96%, at least 97%, at least 98%, at least 99%, or 100% identical to the amino acid sequence of SEQ ID NO:45, and V_{L1} comprises an amino acid sequence that is at least 95%, at least 96%, at least 97%, at least 98%, at least 99%, or 100% identical to the amino acid sequence of SEQ ID NO:43.
   Embodiment 6. The binding protein of embodiment 1 or embodiment 2, wherein the binding site that specifically binds an IL-4 polypeptide comprises:
(a) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24, and a VH domain that comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10;
(b) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11;
(c) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, and a VH domain that comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10;
(d) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11;
(e) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:26, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11; or
(f) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:27, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11.
   Embodiment 7. The binding protein of embodiment 6, wherein the binding site that specifically binds an IL-4 polypeptide comprises:
(a) a VH domain that comprises an amino acid sequence that is at least 95%, at least 96%, at least 97%, at least 98%, at least 99%, or 100% identical to the amino acid sequence of SEQ ID NO:46 and a VH domain that comprises an amino acid sequence that is at least 95%, at least 96%, at least 97%, at least 98%, at least 99%, or 100% identical to the amino acid sequence of SEQ ID NO:47;
(b) a VH domain that comprises an amino acid sequence that is at least 95%, at least 96%, at least 97%, at least 98%, at least 99%, or 100% identical to the amino acid sequence of SEQ ID NO:48 and a VH domain that comprises an amino acid sequence that is at least 95%, at least 96%, at least 97%, at least 98%, at least 99%, or 100% identical to the amino acid sequence of SEQ ID NO:39;
(c) a VH domain that comprises an amino acid sequence that is at least 95%, at least 96%, at least 97%, at least 98%, at least 99%, or 100% identical to the amino acid sequence of SEQ ID NO:49 and a VH domain that comprises an amino acid sequence that is at least 95%, at least 96%, at least 97%, at least 98%, at least 99%, or 100% identical to the amino acid sequence of SEQ ID NO:41;
(d) a VH domain that comprises an amino acid sequence that is at least 95%, at least 96%, at least 97%, at least 98%, at least 99%, or 100% identical to the amino acid sequence of SEQ ID NO:50 and a VH domain that comprises an amino acid sequence that is at least 95%, at least 96%, at least 97%, at least 98%, at least 99%, or 100% identical to the amino acid sequence of SEQ ID NO:43;
(e) a VH domain that comprises an amino acid sequence that is at least 95%, at least 96%, at least 97%, at least 98%, at least 99%, or 100% identical to the amino acid sequence of SEQ ID NO:51 and a VH domain that comprises an amino acid sequence that is at least 95%, at least 96%, at least 97%, at least 98%, at least 99%, or 100% identical to the amino acid sequence of SEQ ID NO:37;
(f) a VH domain that comprises an amino acid sequence that is at least 95%, at least 96%, at least 97%, at least 98%, at least 99%, or 100% identical to the amino acid sequence of SEQ ID NO:52 and a VH domain that comprises an amino acid sequence that is at least 95%, at least 96%, at least 97%, at least 98%, at least 99%, or 100% identical to the amino acid sequence of SEQ ID NO:39;
(g) a VH domain that comprises an amino acid sequence that is at least 95%, at least 96%, at least 97%, at least 98%, at least 99%, or 100% identical to the amino acid sequence of SEQ ID NO:53 and a VH domain that comprises an amino acid sequence that is at least 95%, at least 96%, at least 97%, at least 98%, at least 99%, or 100% identical to the amino acid sequence of SEQ ID NO:41;
(h) a VH domain that comprises an amino acid sequence that is at least 95%, at least 96%, at least 97%, at least 98%, at least 99%, or 100% identical to the amino acid sequence of SEQ ID NO:54 and a VH domain that comprises an amino acid sequence that is at least 95%, at least 96%, at least 97%, at least 98%, at least 99%, or 100% identical to the amino acid sequence of SEQ ID NO:43;
(i) a VH domain that comprises an amino acid sequence that is at least 95%, at least 96%, at least 97%, at least 98%, at least 99%, or 100% identical to the amino acid sequence of SEQ ID NO:55 and a VH domain that comprises an amino acid sequence that is at least 95%, at least 96%, at least 97%, at least 98%, at least 99%, or 100% identical to the amino acid sequence of SEQ ID NO:43; or
(j) a VH domain that comprises an amino acid sequence that is at least 95%, at least 96%, at least 97%, at least 98%, at least 99%, or 100% identical to the amino acid sequence of SEQ ID NO:56 and a VH domain that comprises an amino acid sequence that is at least 95%, at least 96%, at least 97%, at least 98%, at least 99%, or 100% identical to the amino acid sequence of SEQ ID NO:43.
   Embodiment 8. The binding protein of embodiment 7, wherein:
(a) V_{H1} comprises an amino acid sequence that is at least 95%, at least 96%, at least 97%, at least 98%, at least 99%, or 100% identical to the amino acid sequence of SEQ ID NO:46, and V_{L1} comprises an amino acid sequence that is at least 95%, at least 96%, at least 97%, at least 98%, at least 99%, or 100% identical to the amino acid sequence of SEQ ID NO:47;
(b) V_{H1} comprises an amino acid sequence that is at least 95%, at least 96%, at least 97%, at least 98%, at least 99%, or 100% identical to the amino acid sequence of SEQ ID NO:48, and V_{L1} comprises an amino acid sequence that is at least 95%, at least 96%, at least 97%, at least 98%, at least 99%, or 100% identical to the amino acid sequence of SEQ ID NO:39;
(c) V_{H1} comprises an amino acid sequence that is at least 95%, at least 96%, at least 97%, at least 98%, at least 99%, or 100% identical to the amino acid sequence of SEQ ID NO:49, and V_{L1} comprises an amino acid sequence that is at least 95%, at least 96%, at least 97%, at least 98%, at least 99%, or 100% identical to the amino acid sequence of SEQ ID NO:41;
(d) V_{H1} comprises an amino acid sequence that is at least 95%, at least 96%, at least 97%, at least 98%, at least 99%, or 100% identical to the amino acid sequence of SEQ ID NO:50, and V_{L1} comprises an amino acid sequence that is at least 95%, at least 96%, at least 97%, at least 98%, at least 99%, or 100% identical to the amino acid sequence of SEQ ID NO:43;
(e) V_{H1} comprises an amino acid sequence that is at least 95%, at least 96%, at least 97%, at least 98%, at least 99%, or 100% identical to the amino acid sequence of SEQ ID NO:51, and V_{L1} comprises an amino acid sequence that is at least 95%, at least 96%, at least 97%, at least 98%, at least 99%, or 100% identical to the amino acid sequence of SEQ ID NO:37;
(f) V_{H1} comprises an amino acid sequence that is at least 95%, at least 96%, at least 97%, at least 98%, at least 99%, or 100% identical to the amino acid sequence of SEQ ID NO:52, and V_{L1} comprises an amino acid sequence that is at least 95%, at least 96%, at least 97%, at least 98%, at least 99%, or 100% identical to the amino acid sequence of SEQ ID NO:39;
(g) V_{H1} comprises an amino acid sequence that is at least 95%, at least 96%, at least 97%, at least 98%, at least 99%, or 100% identical to the amino acid sequence of SEQ ID NO:53, and V_{L1} comprises an amino acid sequence that is at least 95%, at least 96%, at least 97%, at least 98%, at least 99%, or 100% identical to the amino acid sequence of SEQ ID NO:41;
(h) V_{H1} comprises an amino acid sequence that is at least 95%, at least 96%, at least 97%, at least 98%, at least 99%, or 100% identical to the amino acid sequence of SEQ ID NO:54, and V_{L1} comprises an amino acid sequence that is at least 95%, at least 96%, at least 97%, at least 98%, at least 99%, or 100% identical to the amino acid sequence of SEQ ID NO:43;
(i) V_{H1} comprises an amino acid sequence that is at least 95%, at least 96%, at least 97%, at least 98%, at least 99%, or 100% identical to the amino acid sequence of SEQ ID NO:55, and V_{L1} comprises an amino acid sequence that is at least 95%, at least 96%, at least 97%, at least 98%, at least 99%, or 100% identical to the amino acid sequence of SEQ ID NO:43; or
(j) V_{H1} comprises an amino acid sequence that is at least 95%, at least 96%, at least 97%, at least 98%, at least 99%, or 100% identical to the amino acid sequence of SEQ ID NO:56, and V_{L1} comprises an amino acid sequence that is at least 95%, at least 96%, at least 97%, at least 98%, at least 99%, or 100% identical to the amino acid sequence of SEQ ID NO:43.
   Embodiment 9. The binding protein of any one of embodiments 1-8, wherein the binding site that specifically binds an IL-13 polypeptide comprises: a VH domain that comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, and a VH domain that comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62.
   Embodiment 10. The binding protein of embodiment 9, wherein the binding site that specifically binds an IL-13 polypeptide comprises:
(a) a VH domain that comprises an amino acid sequence that is at least 95%, at least 96%, at least 97%, at least 98%, at least 99%, or 100% identical to the amino acid sequence of SEQ ID NO:70 and a VH domain that comprises an amino acid sequence that is at least 95%, at least 96%, at least 97%, at least 98%, at least 99%, or 100% identical to the amino acid sequence of SEQ ID NO:71;
(b) a VH domain that comprises an amino acid sequence that is at least 95%, at least 96%, at least 97%, at least 98%, at least 99%, or 100% identical to the amino acid sequence of SEQ ID NO:72 and a VH domain that comprises an amino acid sequence that is at least 95%, at least 96%, at least 97%, at least 98%, at least 99%, or 100% identical to the amino acid sequence of SEQ ID NO:73; or
(c) a VH domain that comprises an amino acid sequence that is at least 95%, at least 96%, at least 97%, at least 98%, at least 99%, or 100% identical to the amino acid sequence of SEQ ID NO:74 and a VH domain that comprises an amino acid sequence that is at least 95%, at least 96%, at least 97%, at least 98%, at least 99%, or 100% identical to the amino acid sequence of SEQ ID NO:75.
   Embodiment 11. The binding protein of embodiment 10, wherein:
(a) V_{H2} comprises an amino acid sequence that is at least 95%, at least 96%, at least 97%, at least 98%, at least 99%, or 100% identical to the amino acid sequence of SEQ ID NO:70, and V_{L2} comprises an amino acid sequence that is at least 95%, at least 96%, at least 97%, at least 98%, at least 99%, or 100% identical to the amino acid sequence of SEQ ID NO:71;
(b) V_{H2} comprises an amino acid sequence that is at least 95%, at least 96%, at least 97%, at least 98%, at least 99%, or 100% identical to the amino acid sequence of SEQ ID NO:72, and V_{L2} comprises an amino acid sequence that is at least 95%, at least 96%, at least 97%, at least 98%, at least 99%, or 100% identical to the amino acid sequence of SEQ ID NO:73; or
(c) V_{H2} comprises an amino acid sequence that is at least 95%, at least 96%, at least 97%, at least 98%, at least 99%, or 100% identical to the amino acid sequence of SEQ ID NO:74, and V_{L2} comprises an amino acid sequence that is at least 95%, at least 96%, at least 97%, at least 98%, at least 99%, or 100% identical to the amino acid sequence of SEQ ID NO:75.
   Embodiment 12. The binding protein of any one of embodiments 1-8, wherein the binding site that specifically binds an IL-13 polypeptide comprises: a VH domain that comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:108, and a VH domain that comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62.
   Embodiment 13. The binding protein of embodiment 12, wherein the binding site that specifically binds an IL-13 polypeptide comprises a VH domain that comprises an amino acid sequence that is at least 95%, at least 96%, at least 97%, at least 98%, at least 99%, or 100% identical to the amino acid sequence of SEQ ID NO:76 and a VH domain that comprises an amino acid sequence that is at least 95%, at least 96%, at least 97%, at least 98%, at least 99%, or 100% identical to the amino acid sequence of SEQ ID NO:77.
   Embodiment 14. The binding protein of embodiment 13, wherein V_{H2} comprises an amino acid sequence that is at least 95%, at least 96%, at least 97%, at least 98%, at least 99%, or 100% identical to the amino acid sequence of SEQ ID NO:76, and V_{L2} comprises an amino acid sequence that is at least 95%, at least 96%, at least 97%, at least 98%, at least 99%, or 100% identical to the amino acid sequence of SEQ ID NO:77.
   Embodiment 15. The binding protein of embodiment 1 or embodiment 2, wherein one, two, or all three of the antigen binding sites comprise a pair of reciprocal amino acid substitutions on the V_{H} and V_{L} domains that facilitate heavy-light chain pairing. Embodiment 16. The binding protein of embodiment 1, wherein V_{H1} comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3; V_{L1} comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10; V_{H2} comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59; and V_{L2} comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62.
   Embodiment 17. The binding protein of embodiment 1, wherein V_{H1} comprises the amino acid sequence of SEQ ID NO:34, V_{L1} comprises the amino acid sequence of SEQ ID NO:35, V_{H2} comprises the amino acid sequence of SEQ ID NO:70, and V_{L2} comprises the amino acid sequence of SEQ ID NO:71.
   Embodiment 18. The binding protein of any one of embodiments 1-17, wherein the binding protein is capable of inhibiting the function of one or more target proteins. Embodiment 19. The binding protein of embodiment 18, wherein the binding protein is capable of inhibiting the function of an IL-4 polypeptide and/or an IL-13 polypeptide. Embodiment 20. The binding protein of any one of embodiments 1-19, wherein the third antigen binding site specifically binds an OX40L polypeptide.
   Embodiment 21. The method of embodiment 20, wherein the binding protein is capable of inhibiting the function of an OX40L polypeptide.
   Embodiment 22. The binding protein of embodiment 20 or embodiment 21, wherein V_{H3} comprises a CDR-H1 comprising the amino acid sequence of SEQ ID NO:78, a CDR-H2 comprising the amino acid sequence of SEQ ID NO:79, and a CDR-H3 comprising the amino acid sequence of SEQ ID NO:80; and V_{L3} comprises a CDR-L1 comprising the amino acid sequence of SEQ ID NO:81, a CDR-L2 comprising the amino acid sequence of SEQ ID NO:82, and a CDR-L3 comprising the amino acid sequence of SEQ ID NO:83.
   Embodiment 23. The binding protein of embodiment 22, wherein:
(a) V_{H3} comprises an amino acid sequence that is at least 95%, at least 96%, at least 97%, at least 98%, at least 99%, or 100% identical to the amino acid sequence of SEQ ID NO:91, and V_{L3} comprises an amino acid sequence that is at least 95%, at least 96%, at least 97%, at least 98%, at least 99%, or 100% identical to the amino acid sequence of SEQ ID NO:92;
(b) V_{H3} comprises an amino acid sequence that is at least 95%, at least 96%, at least 97%, at least 98%, at least 99%, or 100% identical to the amino acid sequence of SEQ ID NO:93, and V_{L3} comprises an amino acid sequence that is at least 95%, at least 96%, at least 97%, at least 98%, at least 99%, or 100% identical to the amino acid sequence of SEQ ID NO:94;
(c) V_{H3} comprises an amino acid sequence that is at least 95%, at least 96%, at least 97%, at least 98%, at least 99%, or 100% identical to the amino acid sequence of SEQ ID NO:95, and V_{L3} comprises an amino acid sequence that is at least 95%, at least 96%, at least 97%, at least 98%, at least 99%, or 100% identical to the amino acid sequence of SEQ ID NO:96; or
(d) V_{H3} comprises an amino acid sequence that is at least 95%, at least 96%, at least 97%, at least 98%, at least 99%, or 100% identical to the amino acid sequence of SEQ ID NO:97, and V_{L3} comprises an amino acid sequence that is at least 95%, at least 96%, at least 97%, at least 98%, at least 99%, or 100% identical to the amino acid sequence of SEQ ID NO:96.
   Embodiment 24. The binding protein of embodiment 1, wherein V_{H1} comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3; V_{L1} comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10; V_{H2} comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59; V_{L2} comprises a CDR-L₁ comprising the amino acid sequence of SEQ ID NO:60, a CDR-L2 comprising the amino acid sequence of SEQ ID NO:61, and a CDR-L3 comprising the amino acid sequence of SEQ ID NO:62; V_{H3} comprises a CDR-H1 comprising the amino acid sequence of SEQ ID NO:78, a CDR-H2 comprising the amino acid sequence of SEQ ID NO:79, and a CDR-H3 comprising the amino acid sequence of SEQ ID NO:80; and V_{L3} comprises a CDR-L1 comprising the amino acid sequence of SEQ ID NO:81, a CDR-L2 comprising the amino acid sequence of SEQ ID NO:82, and a CDR-L3 comprising the amino acid sequence of SEQ ID NO:83. Embodiment 25. The binding protein of embodiment 1, wherein VHi comprises the amino acid sequence of SEQ ID NO:34, V_{L1} comprises the amino acid sequence of SEQ ID NO:35, V_{H2} comprises the amino acid sequence of SEQ ID NO:70, V_{L2} comprises the amino acid sequence of SEQ ID NO:71, V_{H3} comprises the amino acid sequence of SEQ ID NO:91, and V_{L3} comprises the amino acid sequence of SEQ ID NO:92.
   Embodiment 26. The binding protein of any one of embodiments 1-25, wherein at least one of L₁, L₂, L₃ or L₄, is independently 0 amino acids in length.
   Embodiment 27. The binding protein of any one of embodiments 1-25, wherein L₁, L₂, L₃ or L₄ are each independently at least one amino acid in length.
   Embodiment 28. The binding protein of any one of embodiments 1-25, wherein (a) L₁, L₂, L₃ and L₄ each independently are zero amino acids in length or comprise a sequence selected from the group consisting of GGGGSGGGGS (SEQ ID NO:102), GGGGSGGGGSGGGGS (SEQ ID NO:103), S, RT, TKGPS (SEQ ID NO:104), GQPKAAP (SEQ ID NO: 105), and GGSGSSGSGG (SEQ ID NO:106); or (b) L₁, L₂, L₃ and L₄ each independently comprise a sequence selected from the group consisting of GGGGSGGGGS (SEQ ID NO: 102), GGGGSGGGGSGGGGS (SEQ ID NO:103), S, RT, TKGPS (SEQ ID NO:104), GQPKAAP (SEQ ID NO: 105), and GGSGSSGSGG (SEQ ID NO:106).
   Embodiment 29. The binding protein of any one of embodiments 1-25, wherein: (a) L₁ comprises the sequence GQPKAAP (SEQ ID NO: 105), L₂ comprises the sequence TKGPS (SEQ ID NO:104), L3 comprises the sequence S, and L₄ comprises the sequence RT;
(b) L₁ comprises the sequence GGGGSGGGGS (SEQ ID NO:102), L₂ comprises the sequence GGGGSGGGGS (SEQ ID NO:102), L₃ is 0 amino acids in length, and L₄ is 0 amino acids in length;
(c) L₁ comprises the sequence GGSGSSGSGG (SEQ ID NO:106), L₂ comprises the sequence GGSGSSGSGG (SEQ ID NO:106), L₃ is 0 amino acids in length, and L₄ is 0 amino acids in length; or
(d) L₁ comprises the sequence GGGGSGGGGSGGGGS (SEQ ID NO:103), L₂ is 0 amino acids in length, L3 comprises the sequence GGGGSGGGGSGGGGS (SEQ ID NO: 103), and L₄ is 0 amino acids in length.
   Embodiment 30. The binding protein of any one of embodiments 1-29, wherein the C_{H3} domain of the second polypeptide chain comprises amino acid substitutions at positions corresponding to positions 354 and 366 of human IgG1 or IgG4 according to EU Index, wherein the amino acid substitutions are S354C and T366W; and wherein the C_{H3} domain of the third polypeptide chain comprises amino acid substitutions at positions corresponding to positions 349, 366, 368, and 407 of human IgG1 or IgG4 according to EU Index, wherein the amino acid substitutions are Y349C, T366S, L368A, and Y407V.
   Embodiment 31. The binding protein of any one of embodiments 1-29, wherein the C_{H3} domain of the second polypeptide chain comprises amino acid substitutions at positions corresponding to positions 349, 366, 368, and 407 of human IgG1 or IgG4 according to EU Index, wherein the amino acid substitutions are Y349C, T366S, L368A, and Y407V; and wherein the C_{H3} domain of the third polypeptide chain comprises amino acid substitutions at positions corresponding to positions 354 and 366 of human IgG1 or IgG4 according to EU Index, wherein the amino acid substitutions are S354C and T366W.
   Embodiment 32. The binding protein of any one of embodiments 1-31, wherein the C_{H3} domains of the second and the third polypeptide chains are human IgG1 or IgG4 C_{H3} domains, and wherein only one of the C_{H3} domains comprises amino acid substitutions at positions corresponding to positions 435 and 436 of human IgG1 or IgG4 according to EU Index, wherein the amino acid substitutions are H435R and Y436F.
   Embodiment 33. The binding protein of any one of embodiments 1-32, wherein the C_{H3} domains of the second and the third polypeptide chains are human IgG1 C_{H3} domains, and wherein the C_{H3} domains each comprise amino acid substitutions at positions corresponding to positions 234 and 235 of human IgG1 according to EU Index, wherein the amino acid substitutions are L234A and L235A.
   Embodiment 34. The binding protein of embodiment 1, wherein the first polypeptide chain of the binding protein comprises an amino acid sequence that is at least 95%, at least 96%, at least 97%, at least 98%, at least 99%, or 100% identical to the amino acid sequence of SEQ ID NO:98, the second polypeptide chain of the binding protein comprises an amino acid sequence that is at least 95%, at least 96%, at least 97%, at least 98%, at least 99%, or 100% identical to the amino acid sequence of SEQ ID NO:99, the third polypeptide chain of the binding protein comprises an amino acid sequence that is at least 95%, at least 96%, at least 97%, at least 98%, at least 99%, or 100% identical to the amino acid sequence of SEQ ID NO:100, and the fourth polypeptide chain of the binding protein comprises an amino acid sequence that is at least 95%, at least 96%, at least 97%, at least 98%, at least 99%, or 100% identical to the amino acid sequence of SEQ ID NO:101.
   Embodiment 35. A kit of polynucleotides, comprising a first polynucleotide that encodes a first polypeptide chain of the binding protein according to any one of embodiments 1-34, a second polynucleotide that encodes a second polypeptide chain of the binding protein according to any one of embodiments 1-34, a third polynucleotide that encodes a third polypeptide chain of the binding protein according to any one of embodiments 1-34, and a fourth polynucleotide that encodes a fourth polypeptide chain of the binding protein according to any one of embodiments 1-34.
   Embodiment 36. An isolated nucleic acid molecule comprising a nucleotide sequence encoding the binding protein of any one of embodiments 1-34.
   Embodiment 37. An expression vector comprising the nucleic acid molecule of embodiment 36.
   Embodiment 38. An isolated host cell comprising the kit of polynucleotides of embodiment 35, the nucleic acid molecule of embodiment 36, or the expression vector of embodiment 37.
   Embodiment 39. The isolated host cell of embodiment 38, wherein the host cell is a mammalian or insect cell.
   Embodiment 40. A method of producing a binding protein, comprising culturing the host cell of embodiment 38 or embodiment 39 such that the binding protein is produced. Embodiment 41. The method of embodiment 40, further comprising recovering the binding protein from the host cell.
   Embodiment 42. A pharmaceutical composition comprising the binding protein of any one of embodiments 1-34 and a pharmaceutically acceptable carrier.
   Embodiment 43. A method of preventing and/or treating an inflammatory disease or disorder in a patient in need thereof, comprising administering to the patient a therapeutically effective amount of at least one binding protein of any one of embodiments 1-34 or the pharmaceutical composition of embodiment 42.
   Embodiment 44. The method of embodiment 43, wherein the inflammatory disease or disorder is atopic dermatitis.
   Embodiment 45. The method of embodiment 43 or embodiment 44, wherein the patient is a human.
   Embodiment 46. The binding protein of any one of embodiments 1-34 or the pharmaceutical composition of embodiment 42 for use in preventing and/or treating an inflammatory disease or disorder in a patient.
   Embodiment 47. The binding protein or pharmaceutical composition for use of embodiment 46, wherein the inflammatory disease or disorder is atopic dermatitis. Embodiment 48. Use of the binding protein of any one of embodiments 1-34 or the pharmaceutical composition of embodiment 42 in the manufacture of a medicament for preventing and/or treating an inflammatory disease or disorder in a patient. Embodiment 49. The use of embodiment 48, wherein the inflammatory disease or disorder is atopic dermatitis.
   Embodiment 50. An antibody that specifically binds an IL-4 polypeptide, wherein the antibody comprises a heavy chain variable (VH) domain and a light chain variable (VH) domain; wherein the VH domain comprises:
(a) a CDR-H1 comprising the amino acid sequence of SEQ ID NO:1, a CDR-H2 comprising the amino acid sequence of SEQ ID NO:2, and a CDR-H3 comprising the amino acid sequence selected from the group consisting of SEQ ID NOs:3-5; or
(b) a CDR-H1 comprising the amino acid sequence of SEQ ID NO:22, a CDR-H2 comprising the amino acid sequence of SEQ ID NO:23, and a CDR-H3 comprising the amino acid sequence selected from the group consisting of SEQ ID NOs:24-27; and wherein the VL domain comprises a CDR-L1 comprising the amino acid sequence of SEQ ID NO:6 or 7, a CDR-L2 comprising the amino acid sequence of SEQ ID NO:8 or 9, and a CDR-L3 comprising the amino acid sequence of SEQ ID NO:10 or 11.
   Embodiment 51. The antibody of embodiment 50, wherein:
(a) the VH domain comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3, and the VL domain comprises a CDR-L₁ comprising the amino acid sequence of SEQ ID NO:6, a CDR-L2 comprising the amino acid sequence of SEQ ID NO:8, and a CDR-L3 comprising the amino acid sequence of SEQ ID NO:10;
(b) the VH domain comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3, and the VL domain comprises a CDR-L₁ comprising the amino acid sequence of SEQ ID NO:7, a CDR-L2 comprising the amino acid sequence of SEQ ID NO:9, and a CDR-L3 comprising the amino acid sequence of SEQ ID NO:11;
(c) the VH domain comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:4, and the VL domain comprises a CDR-L₁ comprising the amino acid sequence of SEQ ID NO:7, a CDR-L2 comprising the amino acid sequence of SEQ ID NO:9, and a CDR-L3 comprising the amino acid sequence of SEQ ID NO:11; or
(d) the VH domain comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:5, and the VL domain comprises a CDR-L₁ comprising the amino acid sequence of SEQ ID NO:7, a CDR-L2 comprising the amino acid sequence of SEQ ID NO:9, and a CDR-L3 comprising the amino acid sequence of SEQ ID NO:11.
   Embodiment 52. The antibody of embodiment 51, wherein:
(a) the VH domain comprises an amino acid sequence that is at least 95%, at least 96%, at least 97%, at least 98%, at least 99%, or 100% identical to the amino acid sequence of SEQ ID NO:34, and the VL domain comprises an amino acid sequence that is at least 95%, at least 96%, at least 97%, at least 98%, at least 99%, or 100% identical to the amino acid sequence of SEQ ID NO:35;
(b) the VH domain comprises an amino acid sequence that is at least 95%, at least 96%, at least 97%, at least 98%, at least 99%, or 100% identical to the amino acid sequence of SEQ ID NO:36, and the VL domain comprises an amino acid sequence that is at least 95%, at least 96%, at least 97%, at least 98%, at least 99%, or 100% identical to the amino acid sequence of SEQ ID NO:37;
(c) the VH domain comprises an amino acid sequence that is at least 95%, at least 96%, at least 97%, at least 98%, at least 99%, or 100% identical to the amino acid sequence of SEQ ID NO:38, and the VL domain comprises an amino acid sequence that is at least 95%, at least 96%, at least 97%, at least 98%, at least 99%, or 100% identical to the amino acid sequence of SEQ ID NO:39;
(d) the VH domain comprises an amino acid sequence that is at least 95%, at least 96%, at least 97%, at least 98%, at least 99%, or 100% identical to the amino acid sequence of SEQ ID NO:40, and the VL domain comprises an amino acid sequence that is at least 95%, at least 96%, at least 97%, at least 98%, at least 99%, or 100% identical to the amino acid sequence of SEQ ID NO:41;
(e) the VH domain comprises an amino acid sequence that is at least 95%, at least 96%, at least 97%, at least 98%, at least 99%, or 100% identical to the amino acid sequence of SEQ ID NO:42, and the VL domain comprises an amino acid sequence that is at least 95%, at least 96%, at least 97%, at least 98%, at least 99%, or 100% identical to the amino acid sequence of SEQ ID NO:43;
(f) the VH domain comprises an amino acid sequence that is at least 95%, at least 96%, at least 97%, at least 98%, at least 99%, or 100% identical to the amino acid sequence of SEQ ID NO:44, and the VL domain comprises an amino acid sequence that is at least 95%, at least 96%, at least 97%, at least 98%, at least 99%, or 100% identical to the amino acid sequence of SEQ ID NO:43; or
(g) the VH domain comprises an amino acid sequence that is at least 95%, at least 96%, at least 97%, at least 98%, at least 99%, or 100% identical to the amino acid sequence of SEQ ID NO:45, and the VL domain comprises an amino acid sequence that is at least 95%, at least 96%, at least 97%, at least 98%, at least 99%, or 100% identical to the amino acid sequence of SEQ ID NO:43.
   Embodiment 53. The antibody of embodiment 50, wherein:
(a) the VH domain comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24, and the VL domain comprises a CDR-L₁ comprising the amino acid sequence of SEQ ID NO:6, a CDR-L2 comprising the amino acid sequence of SEQ ID NO:8, and a CDR-L3 comprising the amino acid sequence of SEQ ID NO:10;
(b) the VH domain comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24, and the VL domain comprises a CDR-L₁ comprising the amino acid sequence of SEQ ID NO:7, a CDR-L2 comprising the amino acid sequence of SEQ ID NO:9, and a CDR-L3 comprising the amino acid sequence of SEQ ID NO:11;
(c) the VH domain comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, and the VL domain comprises a CDR-L₁ comprising the amino acid sequence of SEQ ID NO:6, a CDR-L2 comprising the amino acid sequence of SEQ ID NO:8, and a CDR-L3 comprising the amino acid sequence of SEQ ID NO:10;
(d) the VH domain comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, and the VL domain comprises a CDR-L₁ comprising the amino acid sequence of SEQ ID NO:7, a CDR-L2 comprising the amino acid sequence of SEQ ID NO:9, and a CDR-L3 comprising the amino acid sequence of SEQ ID NO:11;
(e) the VH domain comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:26, and the VL domain comprises a CDR-L₁ comprising the amino acid sequence of SEQ ID NO:7, a CDR-L2 comprising the amino acid sequence of SEQ ID NO:9, and a CDR-L3 comprising the amino acid sequence of SEQ ID NO:11; or
(f) the VH domain comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:27, and the VL domain comprises a CDR-L₁ comprising the amino acid sequence of SEQ ID NO:7, a CDR-L2 comprising the amino acid sequence of SEQ ID NO:9, and a CDR-L3 comprising the amino acid sequence of SEQ ID NO:11.
   Embodiment 54. The antibody of embodiment 53, wherein:
(a) the VH domain comprises an amino acid sequence that is at least 95%, at least 96%, at least 97%, at least 98%, at least 99%, or 100% identical to the amino acid sequence of SEQ ID NO:46, and the VL domain comprises an amino acid sequence that is at least 95%, at least 96%, at least 97%, at least 98%, at least 99%, or 100% identical to the amino acid sequence of SEQ ID NO:47;
(b) the VH domain comprises an amino acid sequence that is at least 95%, at least 96%, at least 97%, at least 98%, at least 99%, or 100% identical to the amino acid sequence of SEQ ID NO:48, and the VL domain comprises an amino acid sequence that is at least 95%, at least 96%, at least 97%, at least 98%, at least 99%, or 100% identical to the amino acid sequence of SEQ ID NO:39;
(c) the VH domain comprises an amino acid sequence that is at least 95%, at least 96%, at least 97%, at least 98%, at least 99%, or 100% identical to the amino acid sequence of SEQ ID NO:49, and the VL domain comprises an amino acid sequence that is at least 95%, at least 96%, at least 97%, at least 98%, at least 99%, or 100% identical to the amino acid sequence of SEQ ID NO:41;
(d) the VH domain comprises an amino acid sequence that is at least 95%, at least 96%, at least 97%, at least 98%, at least 99%, or 100% identical to the amino acid sequence of SEQ ID NO:50, and the VL domain comprises an amino acid sequence that is at least 95%, at least 96%, at least 97%, at least 98%, at least 99%, or 100% identical to the amino acid sequence of SEQ ID NO:43;
(e) the VH domain comprises an amino acid sequence that is at least 95%, at least 96%, at least 97%, at least 98%, at least 99%, or 100% identical to the amino acid sequence of SEQ ID NO:51, and the VL domain comprises an amino acid sequence that is at least 95%, at least 96%, at least 97%, at least 98%, at least 99%, or 100% identical to the amino acid sequence of SEQ ID NO:37;
(f) the VH domain comprises an amino acid sequence that is at least 95%, at least 96%, at least 97%, at least 98%, at least 99%, or 100% identical to the amino acid sequence of SEQ ID NO:52, and the VL domain comprises an amino acid sequence that is at least 95%, at least 96%, at least 97%, at least 98%, at least 99%, or 100% identical to the amino acid sequence of SEQ ID NO:39;
(g) the VH domain comprises an amino acid sequence that is at least 95%, at least 96%, at least 97%, at least 98%, at least 99%, or 100% identical to the amino acid sequence of SEQ ID NO:53, and the VL domain comprises an amino acid sequence that is at least 95%, at least 96%, at least 97%, at least 98%, at least 99%, or 100% identical to the amino acid sequence of SEQ ID NO:41;
(h) the VH domain comprises an amino acid sequence that is at least 95%, at least 96%, at least 97%, at least 98%, at least 99%, or 100% identical to the amino acid sequence of SEQ ID NO:54, and the VL domain comprises an amino acid sequence that is at least 95%, at least 96%, at least 97%, at least 98%, at least 99%, or 100% identical to the amino acid sequence of SEQ ID NO:43;
(i) the VH domain comprises an amino acid sequence that is at least 95%, at least 96%, at least 97%, at least 98%, at least 99%, or 100% identical to the amino acid sequence of SEQ ID NO:55, and the VL domain comprises an amino acid sequence that is at least 95%, at least 96%, at least 97%, at least 98%, at least 99%, or 100% identical to the amino acid sequence of SEQ ID NO:43; or
(j) the VH domain comprises an amino acid sequence that is at least 95%, at least 96%, at least 97%, at least 98%, at least 99%, or 100% identical to the amino acid sequence of SEQ ID NO:56, and the VL domain comprises an amino acid sequence that is at least 95%, at least 96%, at least 97%, at least 98%, at least 99%, or 100% identical to the amino acid sequence of SEQ ID NO:43.
   Embodiment 55. An antibody that specifically binds an IL-13 polypeptide, wherein the antibody comprises a heavy chain variable (VH) domain and a light chain variable (VH) domain; wherein the VH domain comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, and the VL domain comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62.
   Embodiment 56. The antibody of embodiment 55, wherein:
(a) the VH domain comprises an amino acid sequence that is at least 95%, at least 96%, at least 97%, at least 98%, at least 99%, or 100% identical to the amino acid sequence of SEQ ID NO:70, and the VL domain comprises an amino acid sequence that is at least 95%, at least 96%, at least 97%, at least 98%, at least 99%, or 100% identical to the amino acid sequence of SEQ ID NO:71;
(b) the VH domain comprises an amino acid sequence that is at least 95%, at least 96%, at least 97%, at least 98%, at least 99%, or 100% identical to the amino acid sequence of SEQ ID NO:72, and the VL domain comprises an amino acid sequence that is at least 95%, at least 96%, at least 97%, at least 98%, at least 99%, or 100% identical to the amino acid sequence of SEQ ID NO:73;
(c) the VH domain comprises an amino acid sequence that is at least 95%, at least 96%, at least 97%, at least 98%, at least 99%, or 100% identical to the amino acid sequence of SEQ ID NO:74, and the VL domain comprises an amino acid sequence that is at least 95%, at least 96%, at least 97%, at least 98%, at least 99%, or 100% identical to the amino acid sequence of SEQ ID NO:75; or
(d) the VH domain comprises an amino acid sequence that is at least 95%, at least 96%, at least 97%, at least 98%, at least 99%, or 100% identical to the amino acid sequence of SEQ ID NO:76, and the VL domain comprises an amino acid sequence that is at least 95%, at least 96%, at least 97%, at least 98%, at least 99%, or 100% identical to the amino acid sequence of SEQ ID NO:77.
   Embodiment 57. An antibody that specifically binds an OX40L polypeptide, wherein the antibody comprises a heavy chain variable (VH) domain and a light chain variable (VH) domain; wherein the VH domain comprises a CDR-H1 comprising the amino acid sequence of SEQ ID NO:78, a CDR-H2 comprising the amino acid sequence of SEQ ID NO:79, and a CDR-H3 comprising the amino acid sequence of SEQ ID NO:80, and the VL domain comprises a CDR-L1 comprising the amino acid sequence of SEQ ID NO:81, a CDR-L2 comprising the amino acid sequence of SEQ ID NO:82, and a CDR-L3 comprising the amino acid sequence of SEQ ID NO:83.
   Embodiment 58. The antibody of embodiment 57, wherein:
(a) the VH domain comprises an amino acid sequence that is at least 95%, at least 96%, at least 97%, at least 98%, at least 99%, or 100% identical to the amino acid sequence of SEQ ID NO:91, and the VL domain comprises an amino acid sequence that is at least 95%, at least 96%, at least 97%, at least 98%, at least 99%, or 100% identical to the amino acid sequence of SEQ ID NO:92;
(b) the VH domain comprises an amino acid sequence that is at least 95%, at least 96%, at least 97%, at least 98%, at least 99%, or 100% identical to the amino acid sequence of SEQ ID NO:93, and the VL domain comprises an amino acid sequence that is at least 95%, at least 96%, at least 97%, at least 98%, at least 99%, or 100% identical to the amino acid sequence of SEQ ID NO:94;
(c) the VH domain comprises an amino acid sequence that is at least 95%, at least 96%, at least 97%, at least 98%, at least 99%, or 100% identical to the amino acid sequence of SEQ ID NO:95, and the VL domain comprises an amino acid sequence that is at least 95%, at least 96%, at least 97%, at least 98%, at least 99%, or 100% identical to the amino acid sequence of SEQ ID NO:96; or
(d) the VH domain comprises an amino acid sequence that is at least 95%, at least 96%, at least 97%, at least 98%, at least 99%, or 100% identical to the amino acid sequence of SEQ ID NO:97, and the VL domain comprises an amino acid sequence that is at least 95%, at least 96%, at least 97%, at least 98%, at least 99%, or 100% identical to the amino acid sequence of SEQ ID NO:96.
   Embodiment 59. The antibody of any one of embodiments 50-58, wherein the antibody is a monospecific antibody.
   Embodiment 60. The antibody of any one of embodiments 50-58, wherein the antibody is a bispecific or multispecific antibody.
   Embodiment 61. An isolated nucleic acid molecule comprising a nucleotide sequence encoding the antibody of any one of embodiments 50-60.
   Embodiment 62. An expression vector comprising the nucleic acid molecule of embodiment 61.
   Embodiment 63. An isolated host cell comprising the nucleic acid molecule of embodiment 61 or the expression vector of embodiment 62.
   Embodiment 64. The isolated host cell of embodiment 63, wherein the host cell is a mammalian or insect cell.
   Embodiment 65. A method of producing an antibody, comprising culturing the host cell of embodiment 63 or embodiment 64 such that the antibody is produced.
   Embodiment 66. The method of embodiment 65, further comprising recovering the antibody from the host cell.
   Embodiment 67. A pharmaceutical composition comprising the antibody of any one of embodiments 50-60 and a pharmaceutically acceptable carrier.
   Embodiment 68. A trispecific, trivalent, multispecific and/or multivalent binding protein comprising four polypeptide chains that form three antigen binding sites, wherein a first polypeptide chain of the binding protein comprises a structure represented by the formula:

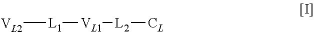

and a second polypeptide chain of the binding protein comprises a structure represented by the formula:

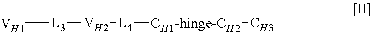

and a third polypeptide chain of the binding protein comprises a structure represented by the formula:

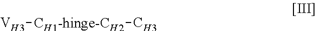

and a fourth polypeptide chain of the binding protein comprises a structure represented by the formula:

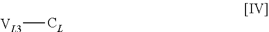

wherein:
   V_{L1} is a first immunoglobulin light chain variable domain;
   V_{L2} is a second immunoglobulin light chain variable domain;
   V_{L3} is a third immunoglobulin light chain variable domain;
   V_{H1} is a first immunoglobulin heavy chain variable domain;
   V_{H2} is a second immunoglobulin heavy chain variable domain;
   V_{H3} is a third immunoglobulin heavy chain variable domain;
   C_{L} is an immunoglobulin light chain constant domain;
   C_{H1} is an immunoglobulin C_{H1} heavy chain constant domain;
   C_{H2} is an immunoglobulin C_{H2} heavy chain constant domain;
   C_{H3} is an immunoglobulin C_{H3} heavy chain constant domain;
   hinge is an immunoglobulin hinge region connecting the C_{H1} and C_{H2} domains; and
   L₁, L₂, L₃ and L₄ are each independently amino acid linkers or zero amino acids in length;
wherein the polypeptide of formula I and the polypeptide of formula II form a cross-over light chain-heavy chain pair of the binding protein; and
wherein V_{H1} and V_{L1} form a binding pair and a first antigen binding site, V_{H2} and V_{L2} form a binding pair and a second antigen binding site, and V_{H3} and V_{L3} form a binding pair and a third antigen binding site;
wherein one of the three antigen binding sites specifically binds an interleukin-17 (IL-17) polypeptide and comprises:
   (a) a heavy chain variable (VH) domain that comprises a CDR-H1 comprising the amino acid sequence of SEQ ID NO:117, a CDR-H2 comprising the amino acid sequence of SEQ ID NO:118, and a CDR-H3 comprising the amino acid sequence of SEQ ID NO:119, and a light chain variable (VH) domain that comprises a CDR-L1 comprising the amino acid sequence of SEQ ID NO: 114, a CDR-L2 comprising the amino acid sequence of TFF, and a CDR-L3 comprising the amino acid sequence of SEQ ID NO:116;
   (b) a heavy chain variable (VH) domain that comprises a CDR-H1 comprising the amino acid sequence of SEQ ID NO:123, a CDR-H2 comprising the amino acid sequence of SEQ ID NO:124, and a CDR-H3 comprising the amino acid sequence of SEQ ID NO:125, and a light chain variable (VH) domain that comprises a CDR-L1 comprising the amino acid sequence of SEQ ID NO:120, a CDR-L2 comprising the amino acid sequence of GTY, and a CDR-L3 comprising the amino acid sequence of SEQ ID NO:122;
   (c) a heavy chain variable (VH) domain that comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131, and a light chain variable (VH) domain that comprises a CDR-L1 comprising the amino acid sequence of SEQ ID NO:126, a CDR-L2 comprising the amino acid sequence of GTY, and a CDR-L3 comprising the amino acid sequence of SEQ ID NO:128;
   (d) a VH domain that contains the same CDR-H1 sequence, the same CDR-H2 sequence, and/or the same CDR-H3 sequence, as the amino acid sequence of SEQ ID NO:132 and a VH domain that contains the same CDR-L1 sequence, the same CDR-L2 sequence, and/or the same CDR-L3 sequence, as the amino acid sequence of SEQ ID NO:133;
   (e) a VH domain that contains the same CDR-H1 sequence, the same CDR-H2 sequence, and/or the same CDR-H3 sequence, as the amino acid sequence of SEQ ID NO:134 and a VH domain that contains the same CDR-L1 sequence, the same CDR-L2 sequence, and/or the same CDR-L3 sequence, as the amino acid sequence of SEQ ID NO:135; or
   (f) a VH domain that contains the same CDR-H1 sequence, the same CDR-H2 sequence, and/or the same CDR-H3 sequence, as the amino acid sequence of SEQ ID NO:136 and a VH domain that contains the same CDR-L1 sequence, the same CDR-L2 sequence, and/or the same CDR-L3 sequence, as the amino acid sequence of SEQ ID NO:137.
      Embodiment 69. The binding protein of embodiment 68, wherein the antigen binding site that specifically binds an IL-17 polypeptide comprises:
(a) a VH domain that comprises an amino acid sequence that is at least 95%, at least 96%, at least 97%, at least 98%, at least 99%, or 100% identical to the amino acid sequence of SEQ ID NO:132 and a VH domain that comprises an amino acid sequence that is at least 95%, at least 96%, at least 97%, at least 98%, at least 99%, or 100% identical to the amino acid sequence of 133;
(b) a VH domain that comprises an amino acid sequence that is at least 95%, at least 96%, at least 97%, at least 98%, at least 99%, or 100% identical to the amino acid sequence of SEQ ID NO:134 and a VH domain that comprises an amino acid sequence that is at least 95%, at least 96%, at least 97%, at least 98%, at least 99%, or 100% identical to the amino acid sequence of 135; or
(c) a VH domain that comprises an amino acid sequence that is at least 95%, at least 96%, at least 97%, at least 98%, at least 99%, or 100% identical to the amino acid sequence of SEQ ID NO:136 and a VH domain that comprises an amino acid sequence that is at least 95%, at least 96%, at least 97%, at least 98%, at least 99%, or 100% identical to the amino acid sequence of 137.
   Embodiment 70. The binding protein of embodiment 68 or embodiment 69, wherein the antigen binding site that specifically binds an IL-17 polypeptide is the third antigen binding site formed by V_{H3} and V_{L3}.
   Embodiment 71. The binding protein of embodiment 70, wherein the first antigen binding site specifically binds an interleukin-4 (IL-4) polypeptide and the second antigen binding site specifically binds an interleukin-13 (IL-13) polypeptide, or the first antigen binding site specifically binds an IL-13 polypeptide and the second antigen binding site specifically binds an IL-4 polypeptide.
   Embodiment 72. The binding protein of embodiment 71, wherein the first binding site specifically binds an IL-4 polypeptide, and the second binding site specifically binds an IL-13 polypeptide.
   Embodiment 73. The binding protein of embodiment 71 or embodiment 72, wherein the binding site that specifically binds an IL-4 polypeptide comprises:
(a) a VH domain that comprises a CDR-H1 comprising the amino acid sequence of SEQ ID NO:12, a CDR-H2 comprising the amino acid sequence of SEQ ID NO:13, and a CDR-H3 comprising the amino acid sequence of SEQ ID NO:14, and a VH domain that comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; or
(b) a VH domain that comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:31, and a VH domain that comprises a CDR-L1 comprising the amino acid sequence of SEQ ID NO:18, a CDR-L2 comprising the amino acid sequence of DAS, and a CDR-L3 comprising the amino acid sequence of SEQ ID NO:21.
   Embodiment 74. The binding protein of embodiment 73, wherein the binding site that specifically binds an IL-4 polypeptide comprises:
(a) a VH domain that comprises an amino acid sequence that is at least 95%, at least 96%, at least 97%, at least 98%, at least 99%, or 100% identical to the amino acid sequence of SEQ ID NO:40 and a VH domain that comprises an amino acid sequence that is at least 95%, at least 96%, at least 97%, at least 98%, at least 99%, or 100% identical to the amino acid sequence of SEQ ID NO:41; or
(b) a VH domain that comprises an amino acid sequence that is at least 95%, at least 96%, at least 97%, at least 98%, at least 99%, or 100% identical to the amino acid sequence of SEQ ID NO:53 and a VH domain that comprises an amino acid sequence that is at least 95%, at least 96%, at least 97%, at least 98%, at least 99%, or 100% identical to the amino acid sequence of SEQ ID NO:41.
   Embodiment 75. The binding protein of any one of embodiments 71-74, wherein the binding site that specifically binds an IL-13 polypeptide comprises a VH domain that comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65, and a VH domain that comprises a CDR-L1 comprising the amino acid sequence of SEQ ID NO:67, a CDR-L2 comprising the amino acid sequence of KDS, and a CDR-L3 comprising the amino acid sequence of SEQ ID NO:69.
   Embodiment 76. The binding protein of embodiment 75, wherein the binding site that specifically binds an IL-13 polypeptide comprises a VH domain that comprises an amino acid sequence that is at least 95%, at least 96%, at least 97%, at least 98%, at least 99%, or 100% identical to the amino acid sequence of SEQ ID NO:72 and a VH domain that comprises an amino acid sequence that is at least 95%, at least 96%, at least 97%, at least 98%, at least 99%, or 100% identical to the amino acid sequence of SEQ ID NO:73.
   Embodiment 77. The binding protein of embodiment 70, wherein:
(a) V_{H1} comprises a CDR-H1 comprising the amino acid sequence of SEQ ID NO:12, a CDR-H2 comprising the amino acid sequence of SEQ ID NO:13, and a CDR-H3 comprising the amino acid sequence of SEQ ID NO:14; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO:123, a CDR-H2 comprising the amino acid sequence of SEQ ID NO: 124, and a CDR-H3 comprising the amino acid sequence of SEQ ID NO:125; and V_{L3} comprises a CDR-L1 comprising the amino acid sequence of SEQ ID NO:120, a CDR-L2 comprising the amino acid sequence of GTY, and a CDR-L3 comprising the amino acid sequence of SEQ ID NO:122;
(b) V_{H1} contains the same CDR-H1 sequence, the same CDR-H2 sequence, and/or the same CDR-H3 sequence, as the amino acid sequence of SEQ ID NO:40 and V_{L1} contains the same CDR-L1 sequence, the same CDR-L2 sequence, and/or the same CDR-L3 sequence, as the amino acid sequence of SEQ ID NO:41; V_{H2} contains the same CDR-H1 sequence, the same CDR-H2 sequence, and/or the same CDR-H3 sequence, as the amino acid sequence of SEQ ID NO:72 and V_{L2} contains the same CDR-L1 sequence, the same CDR-L2 sequence, and/or the same CDR-L3 sequence, as the amino acid sequence of SEQ ID NO:73; and V_{H3} contains the same CDR-H1 sequence, the same CDR-H2 sequence, and/or the same CDR-H3 sequence, as the amino acid sequence of SEQ ID NO:134 and V_{L3} contains the same CDR-L1 sequence, the same CDR-L2 sequence, and/or the same CDR-L3 sequence, as the amino acid sequence of SEQ ID NO:135;
(c) V_{H1} comprises a CDR-H1 comprising the amino acid sequence of SEQ ID NO:12, a CDR-H2 comprising the amino acid sequence of SEQ ID NO:13, and a CDR-H3 comprising the amino acid sequence of SEQ ID NO:14; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO:117, a CDR-H2 comprising the amino acid sequence of SEQ ID NO:118, and a CDR-H3 comprising the amino acid sequence of SEQ ID NO:119; and V_{L3} comprises a CDR-L1 comprising the amino acid sequence of SEQ ID NO:114, a CDR-L2 comprising the amino acid sequence of TFF, and a CDR-L3 comprising the amino acid sequence of SEQ ID NO:116;
(d) V_{H1} contains the same CDR-H1 sequence, the same CDR-H2 sequence, and/or the same CDR-H3 sequence, as the amino acid sequence of SEQ ID NO:40 and V_{L1} contains the same CDR-L1 sequence, the same CDR-L2 sequence, and/or the same CDR-L3 sequence, as the amino acid sequence of SEQ ID NO:41; V_{H2} contains the same CDR-H1 sequence, the same CDR-H2 sequence, and/or the same CDR-H3 sequence, as the amino acid sequence of SEQ ID NO:72 and V_{L2} contains the same CDR-L1 sequence, the same CDR-L2 sequence, and/or the same CDR-L3 sequence, as the amino acid sequence of SEQ ID NO:73; and V_{H3} contains the same CDR-H1 sequence, the same CDR-H2 sequence, and/or the same CDR-H3 sequence, as the amino acid sequence of SEQ ID NO:132 and V_{L3} contains the same CDR-L1 sequence, the same CDR-L2 sequence, and/or the same CDR-L3 sequence, as the amino acid sequence of SEQ ID NO:133;
(e) V_{H1} comprises a CDR-H1 comprising the amino acid sequence of SEQ ID NO:12, a CDR-H2 comprising the amino acid sequence of SEQ ID NO:13, and a CDR-H3 comprising the amino acid sequence of SEQ ID NO:14; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; and V_{L3} comprises a CDR-L1 comprising the amino acid sequence of SEQ ID NO:126, a CDR-L2 comprising the amino acid sequence of GTY, and a CDR-L3 comprising the amino acid sequence of SEQ ID NO:128;
(f) V_{H1} contains the same CDR-H1 sequence, the same CDR-H2 sequence, and/or the same CDR-H3 sequence, as the amino acid sequence of SEQ ID NO:40 and V_{L1} contains the same CDR-L1 sequence, the same CDR-L2 sequence, and/or the same CDR-L3 sequence, as the amino acid sequence of SEQ ID NO:41; V_{H2} contains the same CDR-H1 sequence, the same CDR-H2 sequence, and/or the same CDR-H3 sequence, as the amino acid sequence of SEQ ID NO:72 and V_{L2} contains the same CDR-L1 sequence, the same CDR-L2 sequence, and/or the same CDR-L3 sequence, as the amino acid sequence of SEQ ID NO:73; and V_{H3} contains the same CDR-H1 sequence, the same CDR-H2 sequence, and/or the same CDR-H3 sequence, as the amino acid sequence of SEQ ID NO:136 and V_{L3} contains the same CDR-L1 sequence, the same CDR-L2 sequence, and/or the same CDR-L3 sequence, as the amino acid sequence of SEQ ID NO:137;
(g) V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:31; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO:117, a CDR-H2 comprising the amino acid sequence of SEQ ID NO:118, and a CDR-H3 comprising the amino acid sequence of SEQ ID NO: 119; and V_{L3} comprises a CDR-L1 comprising the amino acid sequence of SEQ ID NO:114, a CDR-L2 comprising the amino acid sequence of TFF, and a CDR-L3 comprising the amino acid sequence of SEQ ID NO:116;
(h) V_{H1} contains the same CDR-H1 sequence, the same CDR-H2 sequence, and/or the same CDR-H3 sequence, as the amino acid sequence of SEQ ID NO:53 and V_{L1} contains the same CDR-L1 sequence, the same CDR-L2 sequence, and/or the same CDR-L3 sequence, as the amino acid sequence of SEQ ID NO:41; V_{H2} contains the same CDR-H1 sequence, the same CDR-H2 sequence, and/or the same CDR-H3 sequence, as the amino acid sequence of SEQ ID NO:72 and V_{L2} contains the same CDR-L1 sequence, the same CDR-L2 sequence, and/or the same CDR-L3 sequence, as the amino acid sequence of SEQ ID NO:73; and V_{H3} contains the same CDR-H1 sequence, the same CDR-H2 sequence, and/or the same CDR-H3 sequence, as the amino acid sequence of SEQ ID NO:132 and V_{L3} contains the same CDR-L1 sequence, the same CDR-L2 sequence, and/or the same CDR-L3 sequence, as the amino acid sequence of SEQ ID NO:133;
(i) V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:31; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO:123, a CDR-H2 comprising the amino acid sequence of SEQ ID NO: 124, and a CDR-H3 comprising the amino acid sequence of SEQ ID NO:125; and V_{L3} comprises a CDR-L1 comprising the amino acid sequence of SEQ ID NO:120, a CDR-L2 comprising the amino acid sequence of GTY, and a CDR-L3 comprising the amino acid sequence of SEQ ID NO:122;
(j) V_{H1} contains the same CDR-H1 sequence, the same CDR-H2 sequence, and/or the same CDR-H3 sequence, as the amino acid sequence of SEQ ID NO:53 and V_{L1} contains the same CDR-L1 sequence, the same CDR-L2 sequence, and/or the same CDR-L3 sequence, as the amino acid sequence of SEQ ID NO:41; V_{H2} contains the same CDR-H1 sequence, the same CDR-H2 sequence, and/or the same CDR-H3 sequence, as the amino acid sequence of SEQ ID NO:72 and V_{L2} contains the same CDR-L1 sequence, the same CDR-L2 sequence, and/or the same CDR-L3 sequence, as the amino acid sequence of SEQ ID NO:73; and V_{H3} contains the same CDR-H1 sequence, the same CDR-H2 sequence, and/or the same CDR-H3 sequence, as the amino acid sequence of SEQ ID NO:134 and V_{L3} contains the same CDR-L1 sequence, the same CDR-L2 sequence, and/or the same CDR-L3 sequence, as the amino acid sequence of SEQ ID NO:135;
(k) V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:31; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; and V_{L3} comprises a CDR-L1 comprising the amino acid sequence of SEQ ID NO:126, a CDR-L2 comprising the amino acid sequence of GTY, and a CDR-L3 comprising the amino acid sequence of SEQ ID NO:128; or
(1) V_{H1} contains the same CDR-H1 sequence, the same CDR-H2 sequence, and/or the same CDR-H3 sequence, as the amino acid sequence of SEQ ID NO:53 and V_{L1} contains the same CDR-L1 sequence, the same CDR-L2 sequence, and/or the same CDR-L3 sequence, as the amino acid sequence of SEQ ID NO:41; V_{H2} contains the same CDR-H1 sequence, the same CDR-H2 sequence, and/or the same CDR-H3 sequence, as the amino acid sequence of SEQ ID NO:72 and V_{L2} contains the same CDR-L1 sequence, the same CDR-L2 sequence, and/or the same CDR-L3 sequence, as the amino acid sequence of SEQ ID NO:73; and V_{H3} contains the same CDR-H1 sequence, the same CDR-H2 sequence, and/or the same CDR-H3 sequence, as the amino acid sequence of SEQ ID NO:136 and V_{L3} contains the same CDR-L1 sequence, the same CDR-L2 sequence, and/or the same CDR-L3 sequence, as the amino acid sequence of SEQ ID NO:137.
   Embodiment 78. The binding protein of any one of embodiments 68, 69, and 77, wherein one, two, or all three of the antigen binding sites comprise reciprocal amino acid substitutions on the VH and VL domains that facilitate heavy-light chain pairing.
   Embodiment 79. The binding protein of embodiment 77, wherein:
(a) V_{H1} comprises an amino acid sequence that is at least 95%, at least 96%, at least 97%, at least 98%, at least 99%, or 100% identical to the amino acid sequence of SEQ ID NO:40; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:136; and V_{L3} comprises an amino acid sequence that is at least 95%, at least 96%, at least 97%, at least 98%, at least 99%, or 100% identical to the amino acid sequence of SEQ ID NO:137;
(b) V_{H1} comprises an amino acid sequence that is at least 95%, at least 96%, at least 97%, at least 98%, at least 99%, or 100% identical to the amino acid sequence of SEQ ID NO:40; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:134; and V_{L3} comprises an amino acid sequence that is at least 95%, at least 96%, at least 97%, at least 98%, at least 99%, or 100% identical to the amino acid sequence of SEQ ID NO:135;
(c) V_{H1} comprises an amino acid sequence that is at least 95%, at least 96%, at least 97%, at least 98%, at least 99%, or 100% identical to the amino acid sequence of SEQ ID NO:40; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:132; and V_{L3} comprises an amino acid sequence that is at least 95%, at least 96%, at least 97%, at least 98%, at least 99%, or 100% identical to the amino acid sequence of SEQ ID NO:133;
(d) V_{H1} comprises an amino acid sequence that is at least 95%, at least 96%, at least 97%, at least 98%, at least 99%, or 100% identical to the amino acid sequence of SEQ ID NO:53; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:132; and V_{L3} comprises an amino acid sequence that is at least 95%, at least 96%, at least 97%, at least 98%, at least 99%, or 100% identical to the amino acid sequence of SEQ ID NO:133;
(e) V_{H1} comprises an amino acid sequence that is at least 95%, at least 96%, at least 97%, at least 98%, at least 99%, or 100% identical to the amino acid sequence of SEQ ID NO:53; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:134; and V_{L3} comprises an amino acid sequence that is at least 95%, at least 96%, at least 97%, at least 98%, at least 99%, or 100% identical to the amino acid sequence of SEQ ID NO:135; or
(f) VHi comprises an amino acid sequence that is at least 95%, at least 96%, at least 97%, at least 98%, at least 99%, or 100% identical to the amino acid sequence of SEQ ID NO:53; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:136; and V_{L3} comprises an amino acid sequence that is at least 95%, at least 96%, at least 97%, at least 98%, at least 99%, or 100% identical to the amino acid sequence of SEQ ID NO:137.
   Embodiment 80. The binding protein of any one of embodiments 68-79, wherein the binding protein is capable of inhibiting the function of one or more target proteins, and wherein the one or more target proteins comprise an IL-17 polypeptide.
   Embodiment 81. The binding protein of embodiment 80, wherein the binding protein is capable of inhibiting the function of an IL-4 polypeptide, an IL-13 polypeptide, and an IL-17 polypeptide.
   Embodiment 82. The binding protein of any one of embodiments 68-81, wherein at least one of L₁, L₂, L₃ or L₄, is independently 0 amino acids in length.
   Embodiment 83. The binding protein of any one of embodiments 68-81, wherein L₁, L₂, L₃ or L₄ are each independently at least one amino acid in length.
   Embodiment 84. The binding protein of any one of embodiments 68-81, wherein (a) L₁, L₂, L₃ and L₄ each independently are zero amino acids in length or comprise a sequence selected from the group consisting of GGGGSGGGGS (SEQ ID NO:102), GGGGSGGGGSGGGGS (SEQ ID NO:103), S, RT, TKGPS (SEQ ID NO:104), GQPKAAP (SEQ ID NO: 105), and GGSGSSGSGG (SEQ ID NO:106); or (b) L₁, L₂, L₃ and L₄ each independently comprise a sequence selected from the group consisting of GGGGSGGGGS (SEQ ID NO: 102), GGGGSGGGGSGGGGS (SEQ ID NO:103), S, RT, TKGPS (SEQ ID NO:104), GQPKAAP (SEQ ID NO: 105), and GGSGSSGSGG (SEQ ID NO:106).
   Embodiment 85. The binding protein of any one of embodiments 68-81, wherein:
(a) L₁ comprises the sequence GQPKAAP (SEQ ID NO: 105), L₂ comprises the sequence TKGPS (SEQ ID NO:104), L3 comprises the sequence S, and L₄ comprises the sequence RT;
(b) L₁ comprises the sequence GGGGSGGGGS (SEQ ID NO:102), L₂ comprises the sequence GGGGSGGGGS (SEQ ID NO:102), L₃ is 0 amino acids in length, and L₄ is 0 amino acids in length;
(c) L₁ comprises the sequence GGSGSSGSGG (SEQ ID NO:106), L₂ comprises the sequence GGSGSSGSGG (SEQ ID NO:106), L₃ is 0 amino acids in length, and L₄ is 0 amino acids in length; or
(d) L₁ comprises the sequence GGGGSGGGGSGGGGS (SEQ ID NO:103), L₂ is 0 amino acids in length, L3 comprises the sequence GGGGSGGGGSGGGGS (SEQ ID NO: 103), and L₄ is 0 amino acids in length.
   Embodiment 86. The binding protein of any one of embodiments 68-85, wherein the C_{H3} domain of the second polypeptide chain comprises amino acid substitutions at positions corresponding to positions 354 and 366 of human IgG1 or IgG4 according to EU Index, wherein the amino acid substitutions are S354C and T366W; and wherein the C_{H3} domain of the third polypeptide chain comprises amino acid substitutions at positions corresponding to positions 349, 366, 368, and 407 of human IgG1 or IgG4 according to EU Index, wherein the amino acid substitutions are Y349C, T366S, L368A, and Y407V.
   Embodiment 87. The binding protein of any one of embodiments 68-85, wherein the C_{H3} domain of the second polypeptide chain comprises amino acid substitutions at positions corresponding to positions 349, 366, 368, and 407 of human IgG1 or IgG4 according to EU Index, wherein the amino acid substitutions are Y349C, T366S, L368A, and Y407V; and wherein the C_{H3} domain of the third polypeptide chain comprises amino acid substitutions at positions corresponding to positions 354 and 366 of human IgG1 or IgG4 according to EU Index, wherein the amino acid substitutions are S354C and T366W.
   Embodiment 88. The binding protein of any one of embodiments 68-87, wherein the C_{H3} domains of the second and the third polypeptide chains are human IgG1 or IgG4 C_{H3} domains, and wherein only one of the C_{H3} domains comprises amino acid substitutions at positions corresponding to positions 435 and 436 of human IgG1 or IgG4 according to EU Index, wherein the amino acid substitutions are H435R and Y436F.
   Embodiment 89. The binding protein of any one of embodiments 68-87, wherein the C_{H3} domains of the second and the third polypeptide chains are human IgG1 C_{H3} domains, and wherein the C_{H3} domains each comprise amino acid substitutions at positions corresponding to positions 234 and 235 of human IgG1 according to EU Index, wherein the amino acid substitutions are L234A and L235A.
   Embodiment 90. The binding protein of embodiment 68, wherein:
(a) the first polypeptide chain of the binding protein comprises an amino acid sequence that is at least 95%, at least 96%, at least 97%, at least 98%, at least 99%, or 100% identical to the amino acid sequence of SEQ ID NO:142, the second polypeptide chain of the binding protein comprises an amino acid sequence that is at least 95%, at least 96%, at least 97%, at least 98%, at least 99%, or 100% identical to the amino acid sequence of SEQ ID NO: 143, the third polypeptide chain of the binding protein comprises an amino acid sequence that is at least 95%, at least 96%, at least 97%, at least 98%, at least 99%, or 100% identical to the amino acid sequence of SEQ ID NO:144, and the fourth polypeptide chain of the binding protein comprises an amino acid sequence that is at least 95%, at least 96%, at least 97%, at least 98%, at least 99%, or 100% identical to the amino acid sequence of SEQ ID NO: 145;
(b) the first polypeptide chain of the binding protein comprises an amino acid sequence that is at least 95%, at least 96%, at least 97%, at least 98%, at least 99%, or 100% identical to the amino acid sequence of SEQ ID NO:138, the second polypeptide chain of the binding protein comprises an amino acid sequence that is at least 95%, at least 96%, at least 97%, at least 98%, at least 99%, or 100% identical to the amino acid sequence of SEQ ID NO: 139, the third polypeptide chain of the binding protein comprises an amino acid sequence that is at least 95%, at least 96%, at least 97%, at least 98%, at least 99%, or 100% identical to the amino acid sequence of SEQ ID NO:140, and the fourth polypeptide chain of the binding protein comprises an amino acid sequence that is at least 95%, at least 96%, at least 97%, at least 98%, at least 99%, or 100% identical to the amino acid sequence of SEQ ID NO:141;
(c) the first polypeptide chain of the binding protein comprises an amino acid sequence that is at least 95%, at least 96%, at least 97%, at least 98%, at least 99%, or 100% identical to the amino acid sequence of SEQ ID NO:146, the second polypeptide chain of the binding protein comprises an amino acid sequence that is at least 95%, at least 96%, at least 97%, at least 98%, at least 99%, or 100% identical to the amino acid sequence of SEQ ID NO: 147, the third polypeptide chain of the binding protein comprises an amino acid sequence that is at least 95%, at least 96%, at least 97%, at least 98%, at least 99%, or 100% identical to the amino acid sequence of SEQ ID NO:148, and the fourth polypeptide chain of the binding protein comprises an amino acid sequence that is at least 95%, at least 96%, at least 97%, at least 98%, at least 99%, or 100% identical to the amino acid sequence of SEQ ID NO: 149;
(d) the first polypeptide chain of the binding protein comprises an amino acid sequence that is at least 95%, at least 96%, at least 97%, at least 98%, at least 99%, or 100% identical to the amino acid sequence of SEQ ID NO:150, the second polypeptide chain of the binding protein comprises an amino acid sequence that is at least 95%, at least 96%, at least 97%, at least 98%, at least 99%, or 100% identical to the amino acid sequence of SEQ ID NO:151, the third polypeptide chain of the binding protein comprises an amino acid sequence that is at least 95%, at least 96%, at least 97%, at least 98%, at least 99%, or 100% identical to the amino acid sequence of SEQ ID NO:152, and the fourth polypeptide chain of the binding protein comprises an amino acid sequence that is at least 95%, at least 96%, at least 97%, at least 98%, at least 99%, or 100% identical to the amino acid sequence of SEQ ID NO: 153;
(e) the first polypeptide chain of the binding protein comprises the amino acid sequence of SEQ ID NO:142, the second polypeptide chain of the binding protein comprises the amino acid sequence of SEQ ID NO:143, the third polypeptide chain of the binding protein comprises the amino acid sequence of SEQ ID NO:144, and the fourth polypeptide chain of the binding protein comprises the amino acid sequence of SEQ ID NO:145;
(f) the first polypeptide chain of the binding protein comprises the amino acid sequence of SEQ ID NO:138, the second polypeptide chain of the binding protein comprises the amino acid sequence of SEQ ID NO:139, the third polypeptide chain of the binding protein comprises the amino acid sequence of SEQ ID NO:140, and the fourth polypeptide chain of the binding protein comprises the amino acid sequence of SEQ ID NO:141;
(g) the first polypeptide chain of the binding protein comprises the amino acid sequence of SEQ ID NO:146, the second polypeptide chain of the binding protein comprises the amino acid sequence of SEQ ID NO:147, the third polypeptide chain of the binding protein comprises the amino acid sequence of SEQ ID NO:148, and the fourth polypeptide chain of the binding protein comprises the amino acid sequence of SEQ ID NO:149; or
(h) the first polypeptide chain of the binding protein comprises the amino acid sequence of SEQ ID NO:150, the second polypeptide chain of the binding protein comprises the amino acid sequence of SEQ ID NO:151, the third polypeptide chain of the binding protein comprises the amino acid sequence of SEQ ID NO:152, and the fourth polypeptide chain of the binding protein comprises the amino acid sequence of SEQ ID NO:153.
   Embodiment 91. A kit of polynucleotides, comprising a first polynucleotide that encodes a first polypeptide chain of the binding protein according to any one of embodiments 68-90, a second polynucleotide that encodes a second polypeptide chain of the binding protein according to any one of embodiments 68-90, a third polynucleotide that encodes a third polypeptide chain of the binding protein according to any one of embodiments 68-90, and a fourth polynucleotide that encodes a fourth polypeptide chain of the binding protein according to any one of embodiments 68-90.
   Embodiment 92. An isolated nucleic acid molecule comprising a nucleotide sequence encoding the binding protein of any one of embodiments 68-90.
   Embodiment 93. An expression vector comprising the nucleic acid molecule of embodiment 92.
   Embodiment 94. An isolated host cell comprising the kit of polynucleotides of embodiment 91, the nucleic acid molecule of embodiment 92, or the expression vector of embodiment 93.
   Embodiment 95. The isolated host cell of embodiment 94, wherein the host cell is a mammalian or insect cell.
   Embodiment 96. A pharmaceutical composition comprising the binding protein of any one of embodiments 68-90 and a pharmaceutically acceptable carrier.
   Embodiment 97. A method of preventing and/or treating and/or alleviating one or more symptoms of an inflammatory disease or disorder in a patient in need thereof, comprising administering to the patient a therapeutically effective amount of at least one binding protein of any one of embodiments 68-90 or the pharmaceutical composition of embodiment 96.
   Embodiment 98. The method of embodiment 97, wherein the inflammatory disease or disorder is atopic dermatitis or chronic obstructive pulmonary disease (COPD).
   Embodiment 99. The method of embodiment 97 or embodiment 98, wherein the patient is a human.
   Embodiment 100. The binding protein of any one of embodiments 68-90 or the pharmaceutical composition of embodiment 96 for use in preventing and/or treating and/or alleviating one or more symptoms of an inflammatory disease or disorder in a patient.
   Embodiment 101. The binding protein or pharmaceutical composition for use of embodiment 100, wherein the inflammatory disease or disorder is atopic dermatitis or chronic obstructive pulmonary disease (COPD).
   Embodiment 102. Use of the binding protein of any one of embodiments 68-90 or the pharmaceutical composition of embodiment 96 in the manufacture of a medicament for preventing and/or treating an inflammatory disease or disorder in a patient.
   Embodiment 103. The use of embodiment 102, wherein the inflammatory disease or disorder is atopic dermatitis or chronic obstructive pulmonary disease (COPD).
   Embodiment 104. An antibody that specifically binds an IL-17 polypeptide, wherein the antibody comprises:
(a) a heavy chain variable (VH) domain that comprises a CDR-H1 comprising the amino acid sequence of SEQ ID NO:117, a CDR-H2 comprising the amino acid sequence of SEQ ID NO:118, and a CDR-H3 comprising the amino acid sequence of SEQ ID NO:119, and a light chain variable (VH) domain that comprises a CDR-L1 comprising the amino acid sequence of SEQ ID NO:114, a CDR-L2 comprising the amino acid sequence of TFF, and a CDR-L3 comprising the amino acid sequence of SEQ ID NO:116;
(b) a heavy chain variable (VH) domain that comprises a CDR-H1 comprising the amino acid sequence of SEQ ID NO:123, a CDR-H2 comprising the amino acid sequence of SEQ ID NO:124, and a CDR-H3 comprising the amino acid sequence of SEQ ID NO:125, and a light chain variable (VH) domain that comprises a CDR-L1 comprising the amino acid sequence of SEQ ID NO:120, a CDR-L2 comprising the amino acid sequence of GTY, and a CDR-L3 comprising the amino acid sequence of SEQ ID NO:122;
(c) a heavy chain variable (VH) domain that comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131, and a light chain variable (VH) domain that comprises a CDR-L1 comprising the amino acid sequence of SEQ ID NO:126, a CDR-L2 comprising the amino acid sequence of GTY, and a CDR-L3 comprising the amino acid sequence of SEQ ID NO:128;
(d) a VH domain that contains the same CDR-H1 sequence, the same CDR-H2 sequence, and/or the same CDR-H3 sequence, as the amino acid sequence of SEQ ID NO:132 and a VH domain that contains the same CDR-L1 sequence, the same CDR-L2 sequence, and/or the same CDR-L3 sequence, as the amino acid sequence of SEQ ID NO:133;
(e) a VH domain that contains the same CDR-H1 sequence, the same CDR-H2 sequence, and/or the same CDR-H3 sequence, as the amino acid sequence of SEQ ID NO:134 and a VH domain that contains the same CDR-L1 sequence, the same CDR-L2 sequence, and/or the same CDR-L3 sequence, as the amino acid sequence of SEQ ID NO:135; or
(f) a VH domain that contains the same CDR-H1 sequence, the same CDR-H2 sequence, and/or the same CDR-H3 sequence, as the amino acid sequence of SEQ ID NO:136 and a VH domain that contains the same CDR-L1 sequence, the same CDR-L2 sequence, and/or the same CDR-L3 sequence, as the amino acid sequence of SEQ ID NO:137.
   Embodiment 105. The antibody of embodiment 104, wherein:
(a) the VH domain comprises an amino acid sequence that is at least 95%, at least 96%, at least 97%, at least 98%, at least 99%, or 100% identical to the amino acid sequence of SEQ ID NO:132 and the VL domain comprises an amino acid sequence that is at least 95%, at least 96%, at least 97%, at least 98%, at least 99%, or 100% identical to the amino acid sequence of 133;
(b) the VH domain comprises an amino acid sequence that is at least 95%, at least 96%, at least 97%, at least 98%, at least 99%, or 100% identical to the amino acid sequence of SEQ ID NO:134 and the VL domain comprises an amino acid sequence that is at least 95%, at least 96%, at least 97%, at least 98%, at least 99%, or 100% identical to the amino acid sequence of 135; or
(c) the VH domain comprises an amino acid sequence that is at least 95%, at least 96%, at least 97%, at least 98%, at least 99%, or 100% identical to the amino acid sequence of SEQ ID NO:136 and the VL domain comprises an amino acid sequence that is at least 95%, at least 96%, at least 97%, at least 98%, at least 99%, or 100% identical to the amino acid sequence of 137.
   Embodiment 106. The antibody of embodiment 104 or embodiment 105, wherein the antibody is a monospecific and/or monovalent antibody.
   Embodiment 107. The antibody of embodiment 104 or embodiment 105, wherein the antibody is a bispecific and/or bivalent antibody, or is a multispecific and/or multivalent antibody.
   Embodiment 108. An isolated nucleic acid molecule comprising a nucleotide sequence encoding the antibody of any one of embodiments 104-107.
   Embodiment 109. An expression vector comprising the nucleic acid molecule of embodiment 108.
   Embodiment 110. An isolated host cell comprising the nucleic acid molecule of embodiment 108 or the expression vector of embodiment 109.
   Embodiment 111. The isolated host cell of embodiment 110, wherein the host cell is a mammalian or insect cell.
   Embodiment 112. A pharmaceutical composition comprising the antibody of any one of embodiments 104-107 and a pharmaceutically acceptable carrier.
   Embodiment 113. A trispecific binding protein that forms three antigen binding sites, wherein one of the antigen binding sites specifically binds an interleukin-4 (IL-4) polypeptide, one of the antigen binding sites specifically binds an interleukin-13 (IL-13) polypeptide, and one of the antigen binding sites specifically binds a polypeptide expressed on the surface of a cytotoxic lymphocyte.
   Embodiment 114. The trispecific binding protein of embodiment 113, wherein the polypeptide is expressed on the surface of a natural killer (NK) cell.
   Embodiment 115. The trispecific binding protein of embodiment 113 or embodiment 114, wherein one of the antigen binding sites specifically binds an IL-4 polypeptide, one of the antigen binding sites specifically binds an IL-13 polypeptide, and one of the antigen binding sites specifically binds a natural killer group 2D (NKG2D) polypeptide.
   Embodiment 116. A trispecific binding protein comprising four polypeptide chains that form three antigen binding sites, wherein a first polypeptide chain of the binding protein comprises a structure represented by the formula:

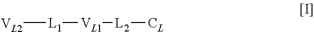

and a second polypeptide chain of the binding protein comprises a structure represented by the formula:

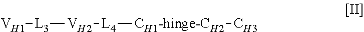

and a third polypeptide chain of the binding protein comprises a structure represented by the formula:

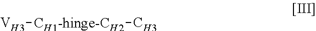

and a fourth polypeptide chain of the binding protein comprises a structure represented by the formula:
wherein:

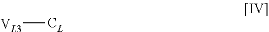

V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are each independently amino acid linkers or zero amino acids in length;
wherein the polypeptide of formula I and the polypeptide of formula II form a cross-over light chain-heavy chain pair of the binding protein; and
wherein V_{H1} and V_{L1} form a binding pair and a first antigen binding site, V_{H2} and V_{L2} form a binding pair and a second antigen binding site, and V_{H3} and V_{L3} form a binding pair and a third antigen binding site;
wherein one of the three antigen binding sites specifically binds an interleukin-4 (IL-4) polypeptide, one of the three antigen binding sites specifically binds an interleukin-13 (IL-13) polypeptide, and one of the three antigen binding sites specifically binds a polypeptide expressed on the surface of cytotoxic lymphocyte.
   Embodiment 117. The binding protein of embodiment 116, wherein the polypeptide is expressed on the surface of a natural killer (NK) cell.
   Embodiment 118. The binding protein of embodiment 116 or embodiment 117, wherein one of the antigen binding sites specifically binds an IL-4 polypeptide, one of the antigen binding sites specifically binds an IL-13 polypeptide, and one of the antigen binding sites specifically binds a natural killer group 2D (NKG2D) polypeptide.
   Embodiment 119. The binding protein of any one of embodiments 116-118, wherein the first antigen binding site specifically binds the IL-4 polypeptide and the second antigen binding site specifically binds the IL-13 polypeptide, or the first antigen binding site specifically binds the IL-13 polypeptide and the second antigen binding site specifically binds the IL-4 polypeptide.
   Embodiment 120. The binding protein of embodiment 119, wherein the first binding site specifically binds the IL-4 polypeptide, and the second binding site specifically binds the IL-13 polypeptide.
   Embodiment 121. The binding protein of any one of embodiments 115-120, wherein the antigen binding site that specifically binds the NKG2D polypeptide comprises:
(a) a VH domain that comprises a CDR-H1 comprising the amino acid sequence of SEQ ID NO:157, a CDR-H2 comprising the amino acid sequence of SEQ ID NO:158, and a CDR-H3 comprising the amino acid sequence of SEQ ID NO:159, and a VH domain that comprises a CDR-L1 comprising the amino acid sequence of SEQ ID NO:160, a CDR-L2 comprising the amino acid sequence of RDS, and a CDR-L3 comprising the amino acid sequence of SEQ ID NO:162;
(b) a VH domain that comprises a CDR-H1 comprising the amino acid sequence of SEQ ID NO:163, a CDR-H2 comprising the amino acid sequence of SEQ ID NO:164, and a CDR-H3 comprising the amino acid sequence of SEQ ID NO:165, and a VH domain that comprises a CDR-L1 comprising the amino acid sequence of SEQ ID NO:166, a CDR-L2 comprising the amino acid sequence of YDD, and a CDR-L3 comprising the amino acid sequence of SEQ ID NO:168;
(c) a VH domain that comprises a CDR-H1 comprising the amino acid sequence of SEQ ID NO:169, a CDR-H2 comprising the amino acid sequence of SEQ ID NO:170, and a CDR-H3 comprising the amino acid sequence of SEQ ID NO:171, and a VH domain that comprises a CDR-L1 comprising the amino acid sequence of SEQ ID NO:172, a CDR-L2 comprising the amino acid sequence of YDD, and a CDR-L3 comprising the amino acid sequence of SEQ ID NO:174;
(d) a VH domain that comprises a CDR-H1 comprising the amino acid sequence of SEQ ID NO:175, a CDR-H2 comprising the amino acid sequence of SEQ ID NO:176, and a CDR-H3 comprising the amino acid sequence of SEQ ID NO:177, and a VH domain that comprises a CDR-L1 comprising the amino acid sequence of SEQ ID NO:178, a CDR-L2 comprising the amino acid sequence of YDD, and a CDR-L3 comprising the amino acid sequence of SEQ ID NO:180;
(e) a VH domain that comprises a CDR-H1 comprising the amino acid sequence of SEQ ID NO:181, a CDR-H2 comprising the amino acid sequence of SEQ ID NO:182, and a CDR-H3 comprising the amino acid sequence of SEQ ID NO:183, and a VH domain that comprises a CDR-L1 comprising the amino acid sequence of SEQ ID NO:184, a CDR-L2 comprising the amino acid sequence of QDT, and a CDR-L3 comprising the amino acid sequence of SEQ ID NO:186;
(f) a VH domain that comprises a CDR-H1 comprising the amino acid sequence of SEQ ID NO:187, a CDR-H2 comprising the amino acid sequence of SEQ ID NO:188, and a CDR-H3 comprising the amino acid sequence of SEQ ID NO:189, and a VH domain that comprises a CDR-L1 comprising the amino acid sequence of SEQ ID NO:190, a CDR-L2 comprising the amino acid sequence of PAS, and a CDR-L3 comprising the amino acid sequence of SEQ ID NO: 192;
(g) a VH domain that comprises a CDR-H1 comprising the amino acid sequence of SEQ ID NO:193, a CDR-H2 comprising the amino acid sequence of SEQ ID NO:194, and a CDR-H3 comprising the amino acid sequence of SEQ ID NO:195, and a VH domain that comprises a CDR-L1 comprising the amino acid sequence of SEQ ID NO:196, a CDR-L2 comprising the amino acid sequence of ATS, and a CDR-L3 comprising the amino acid sequence of SEQ ID NO:198;
(h) a VH domain that comprises a CDR-H1 comprising the amino acid sequence of SEQ ID NO:199, a CDR-H2 comprising the amino acid sequence of SEQ ID NO:200, and a CDR-H3 comprising the amino acid sequence of SEQ ID NO:201, and a VH domain that comprises a CDR-L1 comprising the amino acid sequence of SEQ ID NO:202, a CDR-L2 comprising the amino acid sequence of PAS, and a CDR-L3 comprising the amino acid sequence of SEQ ID NO:204;
(i) a VH domain that comprises a CDR-H1 comprising the amino acid sequence of SEQ ID NO:205, a CDR-H2 comprising the amino acid sequence of SEQ ID NO:206, and a CDR-H3 comprising the amino acid sequence of SEQ ID NO:207, and a VH domain that comprises a CDR-L1 comprising the amino acid sequence of SEQ ID NO:208, a CDR-L2 comprising the amino acid sequence of AAS, and a CDR-L3 comprising the amino acid sequence of SEQ ID NO:210; or
(j) a VH domain that comprises a CDR-H1 comprising the amino acid sequence of SEQ ID NO:211, a CDR-H2 comprising the amino acid sequence of SEQ ID NO:212, and a CDR-H3 comprising the amino acid sequence of SEQ ID NO:213, and a VH domain that comprises a CDR-L1 comprising the amino acid sequence of SEQ ID NO:214, a CDR-L2 comprising the amino acid sequence of YAS, and a CDR-L3 comprising the amino acid sequence of SEQ ID NO:216.
   Embodiment 122. The binding protein of embodiment 121, wherein the antigen binding site that specifically binds the NKG2D polypeptide comprises:
(a) a VH domain that comprises an amino acid sequence that is at least 95%, at least 96%, at least 97%, at least 98%, at least 99%, or 100% identical to the amino acid sequence of SEQ ID NO:217 and a VH domain that comprises an amino acid sequence that is at least 95%, at least 96%, at least 97%, at least 98%, at least 99%, or 100% identical to the amino acid sequence of SEQ ID NO:218;
(b) a VH domain that comprises an amino acid sequence that is at least 95%, at least 96%, at least 97%, at least 98%, at least 99%, or 100% identical to the amino acid sequence of SEQ ID NO:219 and a VH domain that comprises an amino acid sequence that is at least 95%, at least 96%, at least 97%, at least 98%, at least 99%, or 100% identical to the amino acid sequence of SEQ ID NO:220;
(c) a VH domain that comprises an amino acid sequence that is at least 95%, at least 96%, at least 97%, at least 98%, at least 99%, or 100% identical to the amino acid sequence of SEQ ID NO:221 and a VH domain that comprises an amino acid sequence that is at least 95%, at least 96%, at least 97%, at least 98%, at least 99%, or 100% identical to the amino acid sequence of SEQ ID NO:222;
(d) a VH domain that comprises an amino acid sequence that is at least 95%, at least 96%, at least 97%, at least 98%, at least 99%, or 100% identical to the amino acid sequence of SEQ ID NO:223 and a VH domain that comprises an amino acid sequence that is at least 95%, at least 96%, at least 97%, at least 98%, at least 99%, or 100% identical to the amino acid sequence of SEQ ID NO:224;
(e) a VH domain that comprises an amino acid sequence that is at least 95%, at least 96%, at least 97%, at least 98%, at least 99%, or 100% identical to the amino acid sequence of SEQ ID NO:225 and a VH domain that comprises an amino acid sequence that is at least 95%, at least 96%, at least 97%, at least 98%, at least 99%, or 100% identical to the amino acid sequence of SEQ ID NO:226;
(f) a VH domain that comprises an amino acid sequence that is at least 95%, at least 96%, at least 97%, at least 98%, at least 99%, or 100% identical to the amino acid sequence of SEQ ID NO:227 and a VH domain that comprises an amino acid sequence that is at least 95%, at least 96%, at least 97%, at least 98%, at least 99%, or 100% identical to the amino acid sequence of SEQ ID NO:228;
(g) a VH domain that comprises an amino acid sequence that is at least 95%, at least 96%, at least 97%, at least 98%, at least 99%, or 100% identical to the amino acid sequence of SEQ ID NO:229 and a VH domain that comprises an amino acid sequence that is at least 95%, at least 96%, at least 97%, at least 98%, at least 99%, or 100% identical to the amino acid sequence of SEQ ID NO:230;
(h) a VH domain that comprises an amino acid sequence that is at least 95%, at least 96%, at least 97%, at least 98%, at least 99%, or 100% identical to the amino acid sequence of SEQ ID NO:231 and a VH domain that comprises an amino acid sequence that is at least 95%, at least 96%, at least 97%, at least 98%, at least 99%, or 100% identical to the amino acid sequence of SEQ ID NO:232;
(i) a VH domain that comprises an amino acid sequence that is at least 95%, at least 96%, at least 97%, at least 98%, at least 99%, or 100% identical to the amino acid sequence of SEQ ID NO:233 and a VH domain that comprises an amino acid sequence that is at least 95%, at least 96%, at least 97%, at least 98%, at least 99%, or 100% identical to the amino acid sequence of SEQ ID NO:234;
(j) a VH domain that comprises an amino acid sequence that is at least 95%, at least 96%, at least 97%, at least 98%, at least 99%, or 100% identical to the amino acid sequence of SEQ ID NO:235 and a VH domain that comprises an amino acid sequence that is at least 95%, at least 96%, at least 97%, at least 98%, at least 99%, or 100% identical to the amino acid sequence of SEQ ID NO:236;
(k) a VH domain that comprises an amino acid sequence that is at least 95%, at least 96%, at least 97%, at least 98%, at least 99%, or 100% identical to the amino acid sequence of SEQ ID NO:237 and a VH domain that comprises an amino acid sequence that is at least 95%, at least 96%, at least 97%, at least 98%, at least 99%, or 100% identical to the amino acid sequence of SEQ ID NO:238;
(1) a VH domain that comprises an amino acid sequence that is at least 95%, at least 96%, at least 97%, at least 98%, at least 99%, or 100% identical to the amino acid sequence of SEQ ID NO:239 and a VH domain that comprises an amino acid sequence that is at least 95%, at least 96%, at least 97%, at least 98%, at least 99%, or 100% identical to the amino acid sequence of SEQ ID NO:238; or
(m) a VH domain that comprises an amino acid sequence that is at least 95%, at least 96%, at least 97%, at least 98%, at least 99%, or 100% identical to the amino acid sequence of SEQ ID NO:240 and a VH domain that comprises an amino acid sequence that is at least 95%, at least 96%, at least 97%, at least 98%, at least 99%, or 100% identical to the amino acid sequence of SEQ ID NO:241.
   Embodiment 123. The binding protein of embodiment 122, wherein:
(a) V_{H3} comprises an amino acid sequence that is at least 95%, at least 96%, at least 97%, at least 98%, at least 99%, or 100% identical to the amino acid sequence of SEQ ID NO:217 and V_{L3} comprises an amino acid sequence that is at least 95%, at least 96%, at least 97%, at least 98%, at least 99%, or 100% identical to the amino acid sequence of SEQ ID NO:218;
(b) V_{H3} comprises an amino acid sequence that is at least 95%, at least 96%, at least 97%, at least 98%, at least 99%, or 100% identical to the amino acid sequence of SEQ ID NO:219 and V_{L3} comprises an amino acid sequence that is at least 95%, at least 96%, at least 97%, at least 98%, at least 99%, or 100% identical to the amino acid sequence of SEQ ID NO:220;
(c) V_{H3} comprises an amino acid sequence that is at least 95%, at least 96%, at least 97%, at least 98%, at least 99%, or 100% identical to the amino acid sequence of SEQ ID NO:221 and V_{L3} comprises an amino acid sequence that is at least 95%, at least 96%, at least 97%, at least 98%, at least 99%, or 100% identical to the amino acid sequence of SEQ ID NO:222;
(d) V_{H3} comprises an amino acid sequence that is at least 95%, at least 96%, at least 97%, at least 98%, at least 99%, or 100% identical to the amino acid sequence of SEQ ID NO:223 and V_{L3} comprises an amino acid sequence that is at least 95%, at least 96%, at least 97%, at least 98%, at least 99%, or 100% identical to the amino acid sequence of SEQ ID NO:224;
(e) V_{H3} comprises an amino acid sequence that is at least 95%, at least 96%, at least 97%, at least 98%, at least 99%, or 100% identical to the amino acid sequence of SEQ ID NO:225 and V_{L3} comprises an amino acid sequence that is at least 95%, at least 96%, at least 97%, at least 98%, at least 99%, or 100% identical to the amino acid sequence of SEQ ID NO:226;
(f) V_{H3} comprises an amino acid sequence that is at least 95%, at least 96%, at least 97%, at least 98%, at least 99%, or 100% identical to the amino acid sequence of SEQ ID NO:227 and V_{L3} comprises an amino acid sequence that is at least 95%, at least 96%, at least 97%, at least 98%, at least 99%, or 100% identical to the amino acid sequence of SEQ ID NO:228;
(g) V_{H3} comprises an amino acid sequence that is at least 95%, at least 96%, at least 97%, at least 98%, at least 99%, or 100% identical to the amino acid sequence of SEQ ID NO:229 and V_{L3} comprises an amino acid sequence that is at least 95%, at least 96%, at least 97%, at least 98%, at least 99%, or 100% identical to the amino acid sequence of SEQ ID NO:230;
(h) V_{H3} comprises an amino acid sequence that is at least 95%, at least 96%, at least 97%, at least 98%, at least 99%, or 100% identical to the amino acid sequence of SEQ ID NO:231 and V_{L3} comprises an amino acid sequence that is at least 95%, at least 96%, at least 97%, at least 98%, at least 99%, or 100% identical to the amino acid sequence of SEQ ID NO:232;
(i) V_{H3} comprises an amino acid sequence that is at least 95%, at least 96%, at least 97%, at least 98%, at least 99%, or 100% identical to the amino acid sequence of SEQ ID NO:233 and V_{L3} comprises an amino acid sequence that is at least 95%, at least 96%, at least 97%, at least 98%, at least 99%, or 100% identical to the amino acid sequence of SEQ ID NO:234;
(j) V_{H3} comprises an amino acid sequence that is at least 95%, at least 96%, at least 97%, at least 98%, at least 99%, or 100% identical to the amino acid sequence of SEQ ID NO:235 and V_{L3} comprises an amino acid sequence that is at least 95%, at least 96%, at least 97%, at least 98%, at least 99%, or 100% identical to the amino acid sequence of SEQ ID NO:236;
(k) V_{H3} comprises an amino acid sequence that is at least 95%, at least 96%, at least 97%, at least 98%, at least 99%, or 100% identical to the amino acid sequence of SEQ ID NO:237 and V_{L3} comprises an amino acid sequence that is at least 95%, at least 96%, at least 97%, at least 98%, at least 99%, or 100% identical to the amino acid sequence of SEQ ID NO:238;
(1) V_{H3} comprises an amino acid sequence that is at least 95%, at least 96%, at least 97%, at least 98%, at least 99%, or 100% identical to the amino acid sequence of SEQ ID NO:239 and V_{L3} comprises an amino acid sequence that is at least 95%, at least 96%, at least 97%, at least 98%, at least 99%, or 100% identical to the amino acid sequence of SEQ ID NO:238; or
(m) V_{H3} comprises an amino acid sequence that is at least 95%, at least 96%, at least 97%, at least 98%, at least 99%, or 100% identical to the amino acid sequence of SEQ ID NO:240 and V_{L3} comprises an amino acid sequence that is at least 95%, at least 96%, at least 97%, at least 98%, at least 99%, or 100% identical to the amino acid sequence of SEQ ID NO:241.
   Embodiment 124. The binding protein of any one of embodiments 113-123, wherein the antigen binding site that specifically binds the IL-4 polypeptide comprises: (a) a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence selected from the group consisting of SEQ ID NOs:3-5, and a VH domain that comprises a CDR-L1 comprising the amino acid sequence of SEQ ID NO:6 or 7, a CDR-L2 comprising the amino acid sequence of SEQ ID NO:8 or 9, and a CDR-L3 comprising the amino acid sequence of SEQ ID NO:10 or 11; or
(b) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence selected from the group consisting of SEQ ID NOs:24-27, and a VH domain that comprises a CDR-L1 comprising the amino acid sequence of SEQ ID NO:6 or 7, a CDR-L2 comprising the amino acid sequence of SEQ ID NO:8 or 9, and a CDR-L3 comprising the amino acid sequence of SEQ ID NO:10 or 11.
   Embodiment 125. The binding protein of embodiment 124, wherein the binding site that specifically binds an IL-4 polypeptide comprises:
(a) a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3, and a VH domain that comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10;
(b) a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11;
(c) a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:4, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11;
(d) a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:5, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11;
(e) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24, and a VH domain that comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10;
(f) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11;
(g) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, and a VH domain that comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10;
(h) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11;
(i) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:26, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11; or
(j) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:27, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11.
   Embodiment 126. The binding protein of embodiment 125, wherein the binding site that specifically binds an IL-4 polypeptide comprises:
(a) a VH domain that comprises an amino acid sequence that is at least 95%, at least 96%, at least 97%, at least 98%, at least 99%, or 100% identical to the amino acid sequence of SEQ ID NO:34 and a VH domain that comprises an amino acid sequence that is at least 95%, at least 96%, at least 97%, at least 98%, at least 99%, or 100% identical to the amino acid sequence of SEQ ID NO:35;
(b) a VH domain that comprises an amino acid sequence that is at least 95%, at least 96%, at least 97%, at least 98%, at least 99%, or 100% identical to the amino acid sequence of SEQ ID NO:36 and a VH domain that comprises an amino acid sequence that is at least 95%, at least 96%, at least 97%, at least 98%, at least 99%, or 100% identical to the amino acid sequence of SEQ ID NO:37;
(c) a VH domain that comprises an amino acid sequence that is at least 95%, at least 96%, at least 97%, at least 98%, at least 99%, or 100% identical to the amino acid sequence of SEQ ID NO:38 and a VH domain that comprises an amino acid sequence that is at least 95%, at least 96%, at least 97%, at least 98%, at least 99%, or 100% identical to the amino acid sequence of SEQ ID NO:39;
(d) a VH domain that comprises an amino acid sequence that is at least 95%, at least 96%, at least 97%, at least 98%, at least 99%, or 100% identical to the amino acid sequence of SEQ ID NO:40 and a VH domain that comprises an amino acid sequence that is at least 95%, at least 96%, at least 97%, at least 98%, at least 99%, or 100% identical to the amino acid sequence of SEQ ID NO:41;
(e) a VH domain that comprises an amino acid sequence that is at least 95%, at least 96%, at least 97%, at least 98%, at least 99%, or 100% identical to the amino acid sequence of SEQ ID NO:42 and a VH domain that comprises an amino acid sequence that is at least 95%, at least 96%, at least 97%, at least 98%, at least 99%, or 100% identical to the amino acid sequence of SEQ ID NO:43;
(f) a VH domain that comprises an amino acid sequence that is at least 95%, at least 96%, at least 97%, at least 98%, at least 99%, or 100% identical to the amino acid sequence of SEQ ID NO:44 and a VH domain that comprises an amino acid sequence that is at least 95%, at least 96%, at least 97%, at least 98%, at least 99%, or 100% identical to the amino acid sequence of SEQ ID NO:43;
(g) a VH domain that comprises an amino acid sequence that is at least 95%, at least 96%, at least 97%, at least 98%, at least 99%, or 100% identical to the amino acid sequence of SEQ ID NO:45 and a VH domain that comprises an amino acid sequence that is at least 95%, at least 96%, at least 97%, at least 98%, at least 99%, or 100% identical to the amino acid sequence of SEQ ID NO:43;
(h) a VH domain that comprises an amino acid sequence that is at least 95%, at least 96%, at least 97%, at least 98%, at least 99%, or 100% identical to the amino acid sequence of SEQ ID NO:46 and a VH domain that comprises an amino acid sequence that is at least 95%, at least 96%, at least 97%, at least 98%, at least 99%, or 100% identical to the amino acid sequence of SEQ ID NO:47;
(i) a VH domain that comprises an amino acid sequence that is at least 95%, at least 96%, at least 97%, at least 98%, at least 99%, or 100% identical to the amino acid sequence of SEQ ID NO:48 and a VH domain that comprises an amino acid sequence that is at least 95%, at least 96%, at least 97%, at least 98%, at least 99%, or 100% identical to the amino acid sequence of SEQ ID NO:39;
(j) a VH domain that comprises an amino acid sequence that is at least 95%, at least 96%, at least 97%, at least 98%, at least 99%, or 100% identical to the amino acid sequence of SEQ ID NO:49 and a VH domain that comprises an amino acid sequence that is at least 95%, at least 96%, at least 97%, at least 98%, at least 99%, or 100% identical to the amino acid sequence of SEQ ID NO:41;
(k) a VH domain that comprises an amino acid sequence that is at least 95%, at least 96%, at least 97%, at least 98%, at least 99%, or 100% identical to the amino acid sequence of SEQ ID NO:50 and a VH domain that comprises an amino acid sequence that is at least 95%, at least 96%, at least 97%, at least 98%, at least 99%, or 100% identical to the amino acid sequence of SEQ ID NO:43;
(1) a VH domain that comprises an amino acid sequence that is at least 95%, at least 96%, at least 97%, at least 98%, at least 99%, or 100% identical to the amino acid sequence of SEQ ID NO:51 and a VH domain that comprises an amino acid sequence that is at least 95%, at least 96%, at least 97%, at least 98%, at least 99%, or 100% identical to the amino acid sequence of SEQ ID NO:37;
(m) a VH domain that comprises an amino acid sequence that is at least 95%, at least 96%, at least 97%, at least 98%, at least 99%, or 100% identical to the amino acid sequence of SEQ ID NO:52 and a VH domain that comprises an amino acid sequence that is at least 95%, at least 96%, at least 97%, at least 98%, at least 99%, or 100% identical to the amino acid sequence of SEQ ID NO:39;
(n) a VH domain that comprises an amino acid sequence that is at least 95%, at least 96%, at least 97%, at least 98%, at least 99%, or 100% identical to the amino acid sequence of SEQ ID NO:53 and a VH domain that comprises an amino acid sequence that is at least 95%, at least 96%, at least 97%, at least 98%, at least 99%, or 100% identical to the amino acid sequence of SEQ ID NO:41;
(o) a VH domain that comprises an amino acid sequence that is at least 95%, at least 96%, at least 97%, at least 98%, at least 99%, or 100% identical to the amino acid sequence of SEQ ID NO:54 and a VH domain that comprises an amino acid sequence that is at least 95%, at least 96%, at least 97%, at least 98%, at least 99%, or 100% identical to the amino acid sequence of SEQ ID NO:43;
(p) a VH domain that comprises an amino acid sequence that is at least 95%, at least 96%, at least 97%, at least 98%, at least 99%, or 100% identical to the amino acid sequence of SEQ ID NO:55 and a VH domain that comprises an amino acid sequence that is at least 95%, at least 96%, at least 97%, at least 98%, at least 99%, or 100% identical to the amino acid sequence of SEQ ID NO:43; or
(q) a VH domain that comprises an amino acid sequence that is at least 95%, at least 96%, at least 97%, at least 98%, at least 99%, or 100% identical to the amino acid sequence of SEQ ID NO:56 and a VH domain that comprises an amino acid sequence that is at least 95%, at least 96%, at least 97%, at least 98%, at least 99%, or 100% identical to the amino acid sequence of SEQ ID NO:43.
   Embodiment 127. The binding protein of embodiment 126, wherein:
(a) V_{H1} comprises an amino acid sequence that is at least 95%, at least 96%, at least 97%, at least 98%, at least 99%, or 100% identical to the amino acid sequence of SEQ ID NO:34 and V_{L1} comprises an amino acid sequence that is at least 95%, at least 96%, at least 97%, at least 98%, at least 99%, or 100% identical to the amino acid sequence of SEQ ID NO:35;
(b) V_{H1} comprises an amino acid sequence that is at least 95%, at least 96%, at least 97%, at least 98%, at least 99%, or 100% identical to the amino acid sequence of SEQ ID NO:36 and V_{L1} comprises an amino acid sequence that is at least 95%, at least 96%, at least 97%, at least 98%, at least 99%, or 100% identical to the amino acid sequence of SEQ ID NO:37;
(c) V_{H1} comprises an amino acid sequence that is at least 95%, at least 96%, at least 97%, at least 98%, at least 99%, or 100% identical to the amino acid sequence of SEQ ID NO:38 and V_{L1} comprises an amino acid sequence that is at least 95%, at least 96%, at least 97%, at least 98%, at least 99%, or 100% identical to the amino acid sequence of SEQ ID NO:39;
(d) V_{H1} comprises an amino acid sequence that is at least 95%, at least 96%, at least 97%, at least 98%, at least 99%, or 100% identical to the amino acid sequence of SEQ ID NO:40 and V_{L1} comprises an amino acid sequence that is at least 95%, at least 96%, at least 97%, at least 98%, at least 99%, or 100% identical to the amino acid sequence of SEQ ID NO:41;
(e) V_{H1} comprises an amino acid sequence that is at least 95%, at least 96%, at least 97%, at least 98%, at least 99%, or 100% identical to the amino acid sequence of SEQ ID NO:42 and V_{L1} comprises an amino acid sequence that is at least 95%, at least 96%, at least 97%, at least 98%, at least 99%, or 100% identical to the amino acid sequence of SEQ ID NO:43;
(f) V_{H1} comprises an amino acid sequence that is at least 95%, at least 96%, at least 97%, at least 98%, at least 99%, or 100% identical to the amino acid sequence of SEQ ID NO:44 and V_{L1} comprises an amino acid sequence that is at least 95%, at least 96%, at least 97%, at least 98%, at least 99%, or 100% identical to the amino acid sequence of SEQ ID NO:43;
(g) V_{H1} comprises an amino acid sequence that is at least 95%, at least 96%, at least 97%, at least 98%, at least 99%, or 100% identical to the amino acid sequence of SEQ ID NO:45 and V_{L1} comprises an amino acid sequence that is at least 95%, at least 96%, at least 97%, at least 98%, at least 99%, or 100% identical to the amino acid sequence of SEQ ID NO:43;
(h) V_{H1} comprises an amino acid sequence that is at least 95%, at least 96%, at least 97%, at least 98%, at least 99%, or 100% identical to the amino acid sequence of SEQ ID NO:46 and V_{L1} comprises an amino acid sequence that is at least 95%, at least 96%, at least 97%, at least 98%, at least 99%, or 100% identical to the amino acid sequence of SEQ ID NO:47;
(i) V_{H1} comprises an amino acid sequence that is at least 95%, at least 96%, at least 97%, at least 98%, at least 99%, or 100% identical to the amino acid sequence of SEQ ID NO:48 and V_{L1} comprises an amino acid sequence that is at least 95%, at least 96%, at least 97%, at least 98%, at least 99%, or 100% identical to the amino acid sequence of SEQ ID NO:39;
(j) V_{H1} comprises an amino acid sequence that is at least 95%, at least 96%, at least 97%, at least 98%, at least 99%, or 100% identical to the amino acid sequence of SEQ ID NO:49 and V_{L1} comprises an amino acid sequence that is at least 95%, at least 96%, at least 97%, at least 98%, at least 99%, or 100% identical to the amino acid sequence of SEQ ID NO:41;
(k) V_{H1} comprises an amino acid sequence that is at least 95%, at least 96%, at least 97%, at least 98%, at least 99%, or 100% identical to the amino acid sequence of SEQ ID NO:50 and V_{L1} comprises an amino acid sequence that is at least 95%, at least 96%, at least 97%, at least 98%, at least 99%, or 100% identical to the amino acid sequence of SEQ ID NO:43;
(1) V_{H1} comprises an amino acid sequence that is at least 95%, at least 96%, at least 97%, at least 98%, at least 99%, or 100% identical to the amino acid sequence of SEQ ID NO:51 and V_{L1} comprises an amino acid sequence that is at least 95%, at least 96%, at least 97%, at least 98%, at least 99%, or 100% identical to the amino acid sequence of SEQ ID NO:37;
(m) V_{H1} comprises an amino acid sequence that is at least 95%, at least 96%, at least 97%, at least 98%, at least 99%, or 100% identical to the amino acid sequence of SEQ ID NO:52 and V_{L1} comprises an amino acid sequence that is at least 95%, at least 96%, at least 97%, at least 98%, at least 99%, or 100% identical to the amino acid sequence of SEQ ID NO:39;
(n) V_{H1} comprises an amino acid sequence that is at least 95%, at least 96%, at least 97%, at least 98%, at least 99%, or 100% identical to the amino acid sequence of SEQ ID NO:53 and V_{L1} comprises an amino acid sequence that is at least 95%, at least 96%, at least 97%, at least 98%, at least 99%, or 100% identical to the amino acid sequence of SEQ ID NO:41;
(o) V_{H1} comprises an amino acid sequence that is at least 95%, at least 96%, at least 97%, at least 98%, at least 99%, or 100% identical to the amino acid sequence of SEQ ID NO:54 and V_{L1} comprises an amino acid sequence that is at least 95%, at least 96%, at least 97%, at least 98%, at least 99%, or 100% identical to the amino acid sequence of SEQ ID NO:43;
(p) V_{H1} comprises an amino acid sequence that is at least 95%, at least 96%, at least 97%, at least 98%, at least 99%, or 100% identical to the amino acid sequence of SEQ ID NO:55 and V_{L1} comprises an amino acid sequence that is at least 95%, at least 96%, at least 97%, at least 98%, at least 99%, or 100% identical to the amino acid sequence of SEQ ID NO:43; or
(q) V_{H1} comprises an amino acid sequence that is at least 95%, at least 96%, at least 97%, at least 98%, at least 99%, or 100% identical to the amino acid sequence of SEQ ID NO:56 and V_{L1} comprises an amino acid sequence that is at least 95%, at least 96%, at least 97%, at least 98%, at least 99%, or 100% identical to the amino acid sequence of SEQ ID NO:43.
   Embodiment 128. The binding protein of any one of embodiments 113-127, wherein the binding site that specifically binds an IL-13 polypeptide comprises a VH domain that comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59 or 108, and a VH domain that comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62.
   Embodiment 129. The binding protein of embodiment 128, wherein the binding site that specifically binds an IL-13 polypeptide comprises:
(a) a VH domain that comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, and a VH domain that comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62; or
(b) a VH domain that comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:108, and a VH domain that comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62.
   Embodiment 130. The binding protein of embodiment 129, wherein the binding site that specifically binds an IL-13 polypeptide comprises:
(a) a VH domain that comprises an amino acid sequence that is at least 95%, at least 96%, at least 97%, at least 98%, at least 99%, or 100% identical to the amino acid sequence of SEQ ID NO:70 and a VH domain that comprises an amino acid sequence that is at least 95%, at least 96%, at least 97%, at least 98%, at least 99%, or 100% identical to the amino acid sequence of SEQ ID NO:71;
(b) a VH domain that comprises an amino acid sequence that is at least 95%, at least 96%, at least 97%, at least 98%, at least 99%, or 100% identical to the amino acid sequence of SEQ ID NO:72 and a VH domain that comprises an amino acid sequence that is at least 95%, at least 96%, at least 97%, at least 98%, at least 99%, or 100% identical to the amino acid sequence of SEQ ID NO:73;
(c) a VH domain that comprises an amino acid sequence that is at least 95%, at least 96%, at least 97%, at least 98%, at least 99%, or 100% identical to the amino acid sequence of SEQ ID NO:74 and a VH domain that comprises an amino acid sequence that is at least 95%, at least 96%, at least 97%, at least 98%, at least 99%, or 100% identical to the amino acid sequence of SEQ ID NO:75; or
(d) a VH domain that comprises an amino acid sequence that is at least 95%, at least 96%, at least 97%, at least 98%, at least 99%, or 100% identical to the amino acid sequence of SEQ ID NO:76 and a VH domain that comprises an amino acid sequence that is at least 95%, at least 96%, at least 97%, at least 98%, at least 99%, or 100% identical to the amino acid sequence of SEQ ID NO:77.
   Embodiment 131. The binding protein of embodiment 130, wherein:
(a) V_{H2} comprises an amino acid sequence that is at least 95%, at least 96%, at least 97%, at least 98%, at least 99%, or 100% identical to the amino acid sequence of SEQ ID NO:70 and V_{L2} comprises an amino acid sequence that is at least 95%, at least 96%, at least 97%, at least 98%, at least 99%, or 100% identical to the amino acid sequence of SEQ ID NO:71;
(b) V_{H2} comprises an amino acid sequence that is at least 95%, at least 96%, at least 97%, at least 98%, at least 99%, or 100% identical to the amino acid sequence of SEQ ID NO:72 and V_{L2} comprises an amino acid sequence that is at least 95%, at least 96%, at least 97%, at least 98%, at least 99%, or 100% identical to the amino acid sequence of SEQ ID NO:73;
(c) V_{H2} comprises an amino acid sequence that is at least 95%, at least 96%, at least 97%, at least 98%, at least 99%, or 100% identical to the amino acid sequence of SEQ ID NO:74 and V_{L2} comprises an amino acid sequence that is at least 95%, at least 96%, at least 97%, at least 98%, at least 99%, or 100% identical to the amino acid sequence of SEQ ID NO:75; or
(d) V_{H2} comprises an amino acid sequence that is at least 95%, at least 96%, at least 97%, at least 98%, at least 99%, or 100% identical to the amino acid sequence of SEQ ID NO:76 and V_{L2} comprises an amino acid sequence that is at least 95%, at least 96%, at least 97%, at least 98%, at least 99%, or 100% identical to the amino acid sequence of SEQ ID NO:77.
   Embodiment 132. The binding protein of embodiment 116, wherein:
(a) V_{H1} comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, V_{L1} comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11, V_{H2} comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, V_{L2} comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62, V_{H3} comprises a CDR-H1 comprising the amino acid sequence of SEQ ID NO:199, a CDR-H2 comprising the amino acid sequence of SEQ ID NO:200, and a CDR-H3 comprising the amino acid sequence of SEQ ID NO:201, and V_{L3} comprises a CDR-L1 comprising the amino acid sequence of SEQ ID NO:202, a CDR-L₂ comprising the amino acid sequence of PAS, and a CDR-L3 comprising the amino acid sequence of SEQ ID NO:204;
(b) V_{H1} comprises a CDR-H1 comprising the amino acid sequence of SEQ ID NO: 1, a CDR-H2 comprising the amino acid sequence of SEQ ID NO:2, and a CDR-H3 comprising the amino acid sequence of SEQ ID NO:3, V_{L1} comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11, V_{H2} comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, V_{L2} comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62, V_{H3} comprises a CDR-H1 comprising the amino acid sequence of SEQ ID NO:193, a CDR-H2 comprising the amino acid sequence of SEQ ID NO:194, and a CDR-H3 comprising the amino acid sequence of SEQ ID NO:195, and V_{L3} comprises a CDR-L1 comprising the amino acid sequence of SEQ ID NO:196, a CDR-L₂ comprising the amino acid sequence of ATS, and a CDR-L3 comprising the amino acid sequence of SEQ ID NO:198; or
(c) V_{H1} comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, V_{L1} comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11, V_{H2} comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, V_{L2} comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62, V_{H3} comprises a CDR-H1 comprising the amino acid sequence of SEQ ID NO:193, a CDR-H2 comprising the amino acid sequence of SEQ ID NO:194, and a CDR-H3 comprising the amino acid sequence of SEQ ID NO:195, and V_{L3} comprises a CDR-L1 comprising the amino acid sequence of SEQ ID NO:196, a CDR-L₂ comprising the amino acid sequence of ATS, and a CDR-L3 comprising the amino acid sequence of SEQ ID NO:198.
   Embodiment 133. The binding protein of embodiment 116, wherein:
(a) V_{H1} comprises the amino acid sequence of SEQ ID NO:53, V_{L1} comprises the amino acid sequence of SEQ ID NO:41, V_{H2} comprises the amino acid sequence of SEQ ID NO:72, V_{L2} comprises the amino acid sequence of SEQ ID NO:73, V_{H3} comprises the amino acid sequence of SEQ ID NO:240, and V_{L3} comprises the amino acid sequence of SEQ ID NO:241; (b) V_{H1} comprises the amino acid sequence of SEQ ID NO:40, V_{L1} comprises the amino acid sequence of SEQ ID NO:41, V_{H2} comprises the amino acid sequence of SEQ ID NO:72, V_{L2} comprises the amino acid sequence of SEQ ID NO:73, V_{H3} comprises the amino acid sequence of SEQ ID NO:237, and V_{L3} comprises the amino acid sequence of SEQ ID NO:238; or
(c) V_{H1} comprises the amino acid sequence of SEQ ID NO:53, V_{L1} comprises the amino acid sequence of SEQ ID NO:41, V_{H2} comprises the amino acid sequence of SEQ ID NO:72, V_{L2} comprises the amino acid sequence of SEQ ID NO:73, V_{H3} comprises the amino acid sequence of SEQ ID NO:239, and V_{L3} comprises the amino acid sequence of SEQ ID NO:238.
   Embodiment 134. The binding protein of any one of embodiments 113-133, wherein the binding protein is capable of inhibiting the function of one or more target proteins.
   Embodiment 135. The binding protein of embodiment 134, wherein the binding protein is capable of inhibiting the function of an IL-4 polypeptide and/or an IL-13 polypeptide.
   Embodiment 136. The binding protein of embodiment 134 or embodiment 135, wherein the third antigen binding site specifically binds an NKG2D polypeptide, and wherein the binding protein is capable of inhibiting the function of an NKG2D polypeptide.
   Embodiment 137. The binding protein of any one of embodiments 116-136, wherein at least one of L₁, L₂, L₃ or L₄, is independently 0 amino acids in length.
   Embodiment 138. The binding protein of any one of embodiments 116-136, wherein L₁, L₂, L₃ or L₄ are each independently at least one amino acid in length.
   Embodiment 139. The binding protein of any one of embodiments 116-136, wherein (a) L₁, L₂, L₃ and L₄ each independently are zero amino acids in length or comprise a sequence selected from the group consisting of GGGGSGGGGS (SEQ ID NO:102), GGGGSGGGGSGGGGS (SEQ ID NO:103), S, RT, TKGPS (SEQ ID NO:104), GQPKAAP (SEQ ID NO: 105), and GGSGSSGSGG (SEQ ID NO:106); or (b) L₁, L₂, L₃ and L₄ each independently comprise a sequence selected from the group consisting of GGGGSGGGGS (SEQ ID NO:102), GGGGSGGGGSGGGGS (SEQ ID NO:103), S, RT, TKGPS (SEQ ID NO:104), GQPKAAP (SEQ ID NO: 105), and GGSGSSGSGG (SEQ ID NO:106).
   Embodiment 140. The binding protein of any one of embodiments 116-136, wherein: (a) L₁ comprises the sequence GQPKAAP (SEQ ID NO: 105), L₂ comprises the sequence TKGPS (SEQ ID NO:104), L3 comprises the sequence S, and L₄ comprises the sequence RT;
(b) L₁ comprises the sequence GGGGSGGGGS (SEQ ID NO:102), L₂ comprises the sequence GGGGSGGGGS (SEQ ID NO:102), L₃ is 0 amino acids in length, and L₄ is 0 amino acids in length;
(c) L₁ comprises the sequence GGSGSSGSGG (SEQ ID NO:106), L₂ comprises the sequence GGSGSSGSGG (SEQ ID NO:106), L₃ is 0 amino acids in length, and L₄ is 0 amino acids in length; or
(d) L₁ comprises the sequence GGGGSGGGGSGGGGS (SEQ ID NO:103), L₂ is 0 amino acids in length, L3 comprises the sequence GGGGSGGGGSGGGGS (SEQ ID NO: 103), and L₄ is 0 amino acids in length.
   Embodiment 141. The binding protein of any one of embodiments 116-140, wherein the C_{H3} domain of the second polypeptide chain comprises amino acid substitutions at positions corresponding to positions 354 and 366 of human IgG1 or IgG4 according to EU Index, wherein the amino acid substitutions are S354C and T366W; and wherein the C_{H3} domain of the third polypeptide chain comprises amino acid substitutions at positions corresponding to positions 349, 366, 368, and 407 of human IgG1 or IgG4 according to EU Index, wherein the amino acid substitutions are Y349C, T366S, L368A, and Y407V.
   Embodiment 142. The binding protein of any one of embodiments 116-140, wherein the C_{H3} domain of the second polypeptide chain comprises amino acid substitutions at positions corresponding to positions 349, 366, 368, and 407 of human IgG1 or IgG4 according to EU Index, wherein the amino acid substitutions are Y349C, T366S, L368A, and Y407V; and wherein the C_{H3} domain of the third polypeptide chain comprises amino acid substitutions at positions corresponding to positions 354 and 366 of human IgG1 or IgG4 according to EU Index, wherein the amino acid substitutions are S354C and T366W.
   Embodiment 143. The binding protein of any one of embodiments 116-142, wherein the C_{H3} domains of the second and the third polypeptide chains are human IgG1 or IgG4 C_{H3} domains, and wherein only one of the C_{H3} domains comprises amino acid substitutions at positions corresponding to positions 435 and 436 of human IgG1 or IgG4 according to EU Index, wherein the amino acid substitutions are H435R and Y436F.
   Embodiment 144. The binding protein of any one of embodiments 116-143, wherein the C_{H3} domains of the second and the third polypeptide chains are human IgG1 C_{H3} domains, and wherein the C_{H3} domains each comprise amino acid substitutions at positions corresponding to positions 234 and 235 of human IgG1 according to EU Index, wherein the amino acid substitutions are L234A and L235A.
   Embodiment 145. The binding protein of any one of embodiments 116-144, wherein the C_{H3} domains of the second and the third polypeptide chains are human IgG1 C_{H3} domains, and wherein the C_{H3} domains each comprise amino acid substitutions at positions corresponding to positions 256 and 307 of human IgG1 according to EU Index, wherein the amino acid substitutions are T256D and T307Q.
   Embodiment 146. The binding protein of embodiment 116, wherein:
(a) the first polypeptide chain of the binding protein comprises an amino acid sequence that is at least 95%, at least 96%, at least 97%, at least 98%, at least 99%, or 100% identical to the amino acid sequence of SEQ ID NO:242, the second polypeptide chain of the binding protein comprises an amino acid sequence that is at least 95%, at least 96%, at least 97%, at least 98%, at least 99%, or 100% identical to the amino acid sequence of SEQ ID NO:243, the third polypeptide chain of the binding protein comprises an amino acid sequence that is at least 95%, at least 96%, at least 97%, at least 98%, at least 99%, or 100% identical to the amino acid sequence of SEQ ID NO:244, and the fourth polypeptide chain of the binding protein comprises an amino acid sequence that is at least 95%, at least 96%, at least 97%, at least 98%, at least 99%, or 100% identical to the amino acid sequence of SEQ ID NO:245;
(b) the first polypeptide chain of the binding protein comprises an amino acid sequence that is at least 95%, at least 96%, at least 97%, at least 98%, at least 99%, or 100% identical to the amino acid sequence of SEQ ID NO:246, the second polypeptide chain of the binding protein comprises an amino acid sequence that is at least 95%, at least 96%, at least 97%, at least 98%, at least 99%, or 100% identical to the amino acid sequence of SEQ ID NO:247, the third polypeptide chain of the binding protein comprises an amino acid sequence that is at least 95%, at least 96%, at least 97%, at least 98%, at least 99%, or 100% identical to the amino acid sequence of SEQ ID NO:248, and the fourth polypeptide chain of the binding protein comprises an amino acid sequence that is at least 95%, at least 96%, at least 97%, at least 98%, at least 99%, or 100% identical to the amino acid sequence of SEQ ID NO:249; or
(c) the first polypeptide chain of the binding protein comprises an amino acid sequence that is at least 95%, at least 96%, at least 97%, at least 98%, at least 99%, or 100% identical to the amino acid sequence of SEQ ID NO:250, the second polypeptide chain of the binding protein comprises an amino acid sequence that is at least 95%, at least 96%, at least 97%, at least 98%, at least 99%, or 100% identical to the amino acid sequence of SEQ ID NO:251, the third polypeptide chain of the binding protein comprises an amino acid sequence that is at least 95%, at least 96%, at least 97%, at least 98%, at least 99%, or 100% identical to the amino acid sequence of SEQ ID NO:252, and the fourth polypeptide chain of the binding protein comprises an amino acid sequence that is at least 95%, at least 96%, at least 97%, at least 98%, at least 99%, or 100% identical to the amino acid sequence of SEQ ID NO:253.
   Embodiment 147. The binding protein of embodiment 116, wherein:
(a) the first polypeptide chain of the binding protein comprises an amino acid sequence of SEQ ID NO:242, the second polypeptide chain of the binding protein comprises an amino acid sequence of SEQ ID NO:243, the third polypeptide chain of the binding protein comprises an amino acid sequence of SEQ ID NO:244, and the fourth polypeptide chain of the binding protein comprises an amino acid sequence of SEQ ID NO:245;
(b) the first polypeptide chain of the binding protein comprises an amino acid sequence of SEQ ID NO:246, the second polypeptide chain of the binding protein comprises an amino acid sequence of SEQ ID NO:247, the third polypeptide chain of the binding protein comprises an amino acid sequence of SEQ ID NO:248, and the fourth polypeptide chain of the binding protein comprises an amino acid sequence of SEQ ID NO:249; or
(c) the first polypeptide chain of the binding protein comprises an amino acid sequence of SEQ ID NO:250, the second polypeptide chain of the binding protein comprises an amino acid sequence of SEQ ID NO:251, the third polypeptide chain of the binding protein comprises an amino acid sequence of SEQ ID NO:252, and the fourth polypeptide chain of the binding protein comprises an amino acid sequence of SEQ ID NO:253.
   Embodiment 148. A kit of polynucleotides, comprising a first polynucleotide that encodes a first polypeptide chain of the binding protein according to any one of embodiments 116-147, a second polynucleotide that encodes a second polypeptide chain of the binding protein according to any one of embodiments 116-147, a third polynucleotide that encodes a third polypeptide chain of the binding protein according to any one of embodiments 116-147, and a fourth polynucleotide that encodes a fourth polypeptide chain of the binding protein according to any one of embodiments 116-147.
   Embodiment 149. An isolated nucleic acid molecule comprising a nucleotide sequence encoding the binding protein of any one of embodiments 113-147.
   Embodiment 150. An expression vector comprising the nucleic acid molecule of embodiment 149.
   Embodiment 151. An isolated host cell comprising the kit of polynucleotides of embodiment 148, the nucleic acid molecule of embodiment 149, or the expression vector of embodiment 150.
   Embodiment 152. The isolated host cell of embodiment 151, wherein the host cell is a mammalian or insect cell.
   Embodiment 153. A method of producing a trispecific binding protein, comprising culturing the host cell of embodiment 151 or embodiment 152 such that the binding protein is produced.
   Embodiment 154. The method of embodiment 153, further comprising recovering the binding protein from the host cell.
   Embodiment 155. A pharmaceutical composition comprising the binding protein of any one of embodiments 113-147 and a pharmaceutically acceptable carrier.
   Embodiment 156. A method of preventing and/or treating an inflammatory disease or disorder in a patient in need thereof, comprising administering to the patient a therapeutically effective amount of at least one binding protein of any one of embodiments 113-147 or the pharmaceutical composition of embodiment 155.
   Embodiment 157. The method of embodiment 156, wherein the inflammatory disease or disorder is atopic dermatitis, Crohn's disease, asthma, or chronic obstructive pulmonary disease (COPD).
   Embodiment 158. A method of preventing and/or treating cancer in a patient in need thereof, comprising administering to the patient a therapeutically effective amount of at least one binding protein of embodiment 113 or embodiment 114.
   Embodiment 159. The method of any one of embodiments 156-158, wherein the patient is a human.
   Embodiment 160. The binding protein of any one of embodiments 113-147 or the pharmaceutical composition of embodiment 155 for use in preventing and/or treating an inflammatory disease or disorder in a patient.
   Embodiment 161. The binding protein or pharmaceutical composition for use of embodiment 160, wherein the inflammatory disease or disorder is atopic dermatitis, Crohn's disease, asthma, or chronic obstructive pulmonary disease (COPD).
   Embodiment 162. The binding protein of embodiment 113 or embodiment 114 for use in preventing and/or treating cancer in a patient.
   Embodiment 163. Use of the binding protein of any one of embodiments 113-147 or the pharmaceutical composition of embodiment 155 in the manufacture of a medicament for preventing and/or treating an inflammatory disease or disorder in a patient.
   Embodiment 164. The use of embodiment 163, wherein the inflammatory disease or disorder is atopic dermatitis, Crohn's disease, asthma, or chronic obstructive pulmonary disease (COPD).
   Embodiment 165. Use of the binding protein of embodiment 113 or embodiment 114 in the manufacture of a medicament for preventing and/or treating cancer in a patient.
   Embodiment 166. An antibody that specifically binds an NKG2D polypeptide, wherein the antibody comprises a heavy chain variable (VH) domain and a light chain variable (VH) domain; and wherein:
(a) the VH domain comprises a CDR-H1 comprising the amino acid sequence of SEQ ID NO:157, a CDR-H2 comprising the amino acid sequence of SEQ ID NO:158, and a CDR-H3 comprising the amino acid sequence of SEQ ID NO:159, and the VL domain comprises a CDR-L1 comprising the amino acid sequence of SEQ ID NO:160, a CDR-L2 comprising the amino acid sequence of RDS, and a CDR-L3 comprising the amino acid sequence of SEQ ID NO: 162;
(b) the VH domain comprises a CDR-H1 comprising the amino acid sequence of SEQ ID NO:163, a CDR-H2 comprising the amino acid sequence of SEQ ID NO:164, and a CDR-H3 comprising the amino acid sequence of SEQ ID NO:165, and the VL domain comprises a CDR-L1 comprising the amino acid sequence of SEQ ID NO:166, a CDR-L2 comprising the amino acid sequence of YDD, and a CDR-L3 comprising the amino acid sequence of SEQ ID NO: 168;
(c) the VH domain comprises a CDR-H1 comprising the amino acid sequence of SEQ ID NO:169, a CDR-H2 comprising the amino acid sequence of SEQ ID NO:170, and a CDR-H3 comprising the amino acid sequence of SEQ ID NO:171, and the VL domain comprises a CDR-L1 comprising the amino acid sequence of SEQ ID NO:172, a CDR-L2 comprising the amino acid sequence of YDD, and a CDR-L3 comprising the amino acid sequence of SEQ ID NO: 174;
(d) the VH domain comprises a CDR-H1 comprising the amino acid sequence of SEQ ID NO:175, a CDR-H2 comprising the amino acid sequence of SEQ ID NO:176, and a CDR-H3 comprising the amino acid sequence of SEQ ID NO:177, and the VL domain comprises a CDR-L1 comprising the amino acid sequence of SEQ ID NO:178, a CDR-L2 comprising the amino acid sequence of YDD, and a CDR-L3 comprising the amino acid sequence of SEQ ID NO: 180;
(e) the VH domain comprises a CDR-H1 comprising the amino acid sequence of SEQ ID NO:181, a CDR-H2 comprising the amino acid sequence of SEQ ID NO:182, and a CDR-H3 comprising the amino acid sequence of SEQ ID NO:183, and the VL domain comprises a CDR-L1 comprising the amino acid sequence of SEQ ID NO:184, a CDR-L2 comprising the amino acid sequence of QDT, and a CDR-L3 comprising the amino acid sequence of SEQ ID NO: 186;
(f) the VH domain comprises a CDR-H1 comprising the amino acid sequence of SEQ ID NO:187, a CDR-H2 comprising the amino acid sequence of SEQ ID NO:188, and a CDR-H3 comprising the amino acid sequence of SEQ ID NO:189, and the VL domain comprises a CDR-L1 comprising the amino acid sequence of SEQ ID NO:190, a CDR-L2 comprising the amino acid sequence of PAS, and a CDR-L3 comprising the amino acid sequence of SEQ ID NO: 192;
(g) the VH domain comprises a CDR-H1 comprising the amino acid sequence of SEQ ID NO:193, a CDR-H2 comprising the amino acid sequence of SEQ ID NO:194, and a CDR-H3 comprising the amino acid sequence of SEQ ID NO:195, and the VL domain comprises a CDR-L1 comprising the amino acid sequence of SEQ ID NO:196, a CDR-L2 comprising the amino acid sequence of ATS, and a CDR-L3 comprising the amino acid sequence of SEQ ID NO: 198;
(h) the VH domain comprises a CDR-H1 comprising the amino acid sequence of SEQ ID NO:199, a CDR-H2 comprising the amino acid sequence of SEQ ID NO:200, and a CDR-H3 comprising the amino acid sequence of SEQ ID NO:201, and the VL domain comprises a CDR-L1 comprising the amino acid sequence of SEQ ID NO:202, a CDR-L2 comprising the amino acid sequence of PAS, and a CDR-L3 comprising the amino acid sequence of SEQ ID NO:204;
(i) the VH domain comprises a CDR-H1 comprising the amino acid sequence of SEQ ID NO:205, a CDR-H2 comprising the amino acid sequence of SEQ ID NO:206, and a CDR-H3 comprising the amino acid sequence of SEQ ID NO:207, and the VL domain comprises a CDR-L1 comprising the amino acid sequence of SEQ ID NO:208, a CDR-L2 comprising the amino acid sequence of AAS, and a CDR-L3 comprising the amino acid sequence of SEQ ID NO:210; or
(j) the VH domain comprises a CDR-H1 comprising the amino acid sequence of SEQ ID NO:211, a CDR-H2 comprising the amino acid sequence of SEQ ID NO:212, and a CDR-H3 comprising the amino acid sequence of SEQ ID NO:213, and the VL domain comprises a CDR-L1 comprising the amino acid sequence of SEQ ID NO:214, a CDR-L2 comprising the amino acid sequence of YAS, and a CDR-L3 comprising the amino acid sequence of SEQ ID NO:216.
   Embodiment 167. The antibody of embodiment 166, wherein:
(a) the VH domain comprises an amino acid sequence that is at least 95%, at least 96%, at least 97%, at least 98%, at least 99%, or 100% identical to the amino acid sequence of SEQ ID NO:217 and the VL domain comprises an amino acid sequence that is at least 95%, at least 96%, at least 97%, at least 98%, at least 99%, or 100% identical to the amino acid sequence of SEQ ID NO:218;
(b) the VH domain comprises an amino acid sequence that is at least 95%, at least 96%, at least 97%, at least 98%, at least 99%, or 100% identical to the amino acid sequence of SEQ ID NO:219 and the VL domain comprises an amino acid sequence that is at least 95%, at least 96%, at least 97%, at least 98%, at least 99%, or 100% identical to the amino acid sequence of SEQ ID NO:220;
(c) the VH domain comprises an amino acid sequence that is at least 95%, at least 96%, at least 97%, at least 98%, at least 99%, or 100% identical to the amino acid sequence of SEQ ID NO:221 and the VL domain comprises an amino acid sequence that is at least 95%, at least 96%, at least 97%, at least 98%, at least 99%, or 100% identical to the amino acid sequence of SEQ ID NO:222;
(d) the VH domain comprises an amino acid sequence that is at least 95%, at least 96%, at least 97%, at least 98%, at least 99%, or 100% identical to the amino acid sequence of SEQ ID NO:223 and the VL domain comprises an amino acid sequence that is at least 95%, at least 96%, at least 97%, at least 98%, at least 99%, or 100% identical to the amino acid sequence of SEQ ID NO:224;
(e) the VH domain comprises an amino acid sequence that is at least 95%, at least 96%, at least 97%, at least 98%, at least 99%, or 100% identical to the amino acid sequence of SEQ ID NO:225 and the VL domain comprises an amino acid sequence that is at least 95%, at least 96%, at least 97%, at least 98%, at least 99%, or 100% identical to the amino acid sequence of SEQ ID NO:226;
(f) the VH domain comprises an amino acid sequence that is at least 95%, at least 96%, at least 97%, at least 98%, at least 99%, or 100% identical to the amino acid sequence of SEQ ID NO:227 and the VL domain comprises an amino acid sequence that is at least 95%, at least 96%, at least 97%, at least 98%, at least 99%, or 100% identical to the amino acid sequence of SEQ ID NO:228;
(g) the VH domain comprises an amino acid sequence that is at least 95%, at least 96%, at least 97%, at least 98%, at least 99%, or 100% identical to the amino acid sequence of SEQ ID NO:229 and the VL domain comprises an amino acid sequence that is at least 95%, at least 96%, at least 97%, at least 98%, at least 99%, or 100% identical to the amino acid sequence of SEQ ID NO:230;
(h) the VH domain comprises an amino acid sequence that is at least 95%, at least 96%, at least 97%, at least 98%, at least 99%, or 100% identical to the amino acid sequence of SEQ ID NO:231 and the VL domain comprises an amino acid sequence that is at least 95%, at least 96%, at least 97%, at least 98%, at least 99%, or 100% identical to the amino acid sequence of SEQ ID NO:232;
(i) the VH domain comprises an amino acid sequence that is at least 95%, at least 96%, at least 97%, at least 98%, at least 99%, or 100% identical to the amino acid sequence of SEQ ID NO:233 and the VL domain comprises an amino acid sequence that is at least 95%, at least 96%, at least 97%, at least 98%, at least 99%, or 100% identical to the amino acid sequence of SEQ ID NO:234;
(j) the VH domain comprises an amino acid sequence that is at least 95%, at least 96%, at least 97%, at least 98%, at least 99%, or 100% identical to the amino acid sequence of SEQ ID NO:235 and the VL domain comprises an amino acid sequence that is at least 95%, at least 96%, at least 97%, at least 98%, at least 99%, or 100% identical to the amino acid sequence of SEQ ID NO:236;
(k) the VH domain comprises an amino acid sequence that is at least 95%, at least 96%, at least 97%, at least 98%, at least 99%, or 100% identical to the amino acid sequence of SEQ ID NO:237 and the VL domain comprises an amino acid sequence that is at least 95%, at least 96%, at least 97%, at least 98%, at least 99%, or 100% identical to the amino acid sequence of SEQ ID NO:238;
(1) the VH domain comprises an amino acid sequence that is at least 95%, at least 96%, at least 97%, at least 98%, at least 99%, or 100% identical to the amino acid sequence of SEQ ID NO:239 and the VL domain comprises an amino acid sequence that is at least 95%, at least 96%, at least 97%, at least 98%, at least 99%, or 100% identical to the amino acid sequence of SEQ ID NO:238; or
(m) the VH domain comprises an amino acid sequence that is at least 95%, at least 96%, at least 97%, at least 98%, at least 99%, or 100% identical to the amino acid sequence of SEQ ID NO:240 and the VL domain comprises an amino acid sequence that is at least 95%, at least 96%, at least 97%, at least 98%, at least 99%, or 100% identical to the amino acid sequence of SEQ ID NO:241.
   Embodiment 168. The antibody of embodiment 166 or embodiment 167, wherein the antibody is a monospecific antibody.
   Embodiment 169. The antibody of embodiment 166 or embodiment 167, wherein the antibody is a bispecific or multispecific antibody.
   Embodiment 170. An isolated nucleic acid molecule comprising a nucleotide sequence encoding the antibody of any one of embodiments 166-169.
   Embodiment 171. An expression vector comprising the nucleic acid molecule of embodiment 170.
   Embodiment 172. An isolated host cell comprising the nucleic acid molecule of embodiment 170 or the expression vector of embodiment 171.
   Embodiment 173. The isolated host cell of embodiment 172, wherein the host cell is a mammalian or insect cell.
   Embodiment 174. A method of producing an antibody, comprising culturing the host cell of embodiment 172 or embodiment 173 such that the antibody is produced.
   Embodiment 175. The method of embodiment 174, further comprising recovering the antibody from the host cell.
   Embodiment 176. A pharmaceutical composition comprising the antibody of any one of embodiments 166-169 and a pharmaceutically acceptable carrier.

### EXAMPLES

The Examples that follow are illustrative of specific embodiments of the disclosure, and various uses thereof. They are set forth for explanatory purposes only, and should not be construed as limiting the scope of the invention in any way.

### Example 1: Development and Characterization of Bispecific CODV Antibodies Targeting IL-4 and IL-13

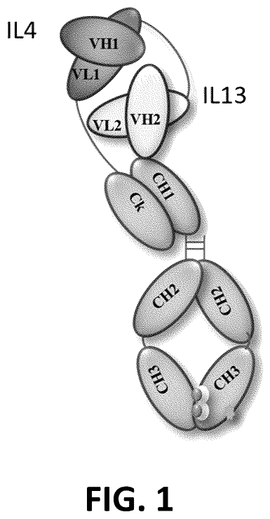

Bispecific antibodies that include one CODV arm with an anti-IL-4 antigen binding domain and an anti-IL-13 antigen binding domain were generated (FIG. 1). To this end, several new antigen binding domains targeting IL-4 and several new antigen binding domains targeting IL-13 were produced by immunizing mice.

### Materials and Methods

### Generation of Anti-IL-4 Antigen Binding Domains

### Antigen Preparation

Antigens were biotinylated using the EZ-Link Sulfo-NHS-Biotinylation Kit (Thermo Scientific, Cat #21425). The antigens were concentrated to −1 mg/mL and buffer exchanged into PBS before addition of 1:7.5 molar ratio biotinylation reagent. The mixture was held at 4° C. overnight prior to another buffer exchange to remove free biotin in the solution. Biotinylation was confirmed through streptavidin sensor binding of the labeled proteins on a ForteBio.

### Naïve Library Selections

Eight naïve human synthetic yeast libraries each of ~-1E9 diversity were propagated as previously described (see, e.g., Y. Xu et al, PEDS 26(10), 663-70 (2013); WO2009036379; WO2010105256; and WO2012009568).

For the first two rounds of selection, a magnetic bead sorting technique utilizing the Miltenyi MACS system was performed, as previously described (see, e.g., Siegel et al, J Immunol Methods 286(1-2), 141-153 (2004).) Briefly, yeast cells (~1E10 cells/library) were incubated with 100 nM biotinylated human IL-4 his for 10 min at 30° C. in wash buffer (phosphate-buffered saline (PBS)/0.1% bovine serum albumin (BSA)). After washing once with 40 mL ice-cold wash buffer, the cell pellet was resuspended in 20 mL wash buffer, and Streptavidin MicroBeads (500 μl) were added to the yeast and incubated for 15 min at 4° C. Next the yeast were pelleted, resuspended in 5 mL wash buffer, and loaded onto a Miltenyi LS column. After the 5 mL were loaded, the column was washed 3 times with 3 mL wash buffer. The column was then removed from the magnetic field, and the yeast were eluted with 5 mL of growth media and then grown overnight.

The following rounds of selection were performed using flow cytometry (FACS). Yeast were pelleted, washed three times with wash buffer, and incubated at 30° C. with either 100 nM biotinylated human IL-4 his, 100 nM biotinylated cyno IL-4 his, 100 nM human IL-13 his, or with a polyspecificity reagent (PSR) to remove non-specific antibodies from the selection. For the PSR depletion, the libraries were incubated with a 1:10 dilution of biotinylated PSR reagent as previously described (see, e.g., Y. Xu et al, PEDS 26(10), 663-70 (2013).) Yeast were then washed twice with wash buffer and stained with goat F(ab′)2 anti-human kappa-FITC (LC-FITC) diluted 1:100 (Southern Biotech, Cat #2062-02) and either Streptavidin-AF633 (SA-633) diluted 1:500 (Life Technologies, Cat #S21375) or Extravidin-phycoerythrin (EA-PE) diluted 1:50 (Sigma-Aldrich, Cat #E4011), secondary reagents for 15 min at 4° C. After washing twice with ice-cold wash buffer, the cell pellets were resuspended in 0.3 mL wash buffer and transferred to strainer-capped sort tubes. Sorting was performed using a FACS ARIA sorter (BD Biosciences) and sort gates were determined to select for antibodies with desired characteristics. Selection rounds were repeated until a population with all the desired characteristics was obtained. After the final round of sorting, yeast were plated and individual colonies were picked for characterization.

### Antibody Optimization

Optimization of antibodies was performed via a light chain batch shuffle, and then by introducing diversities into the heavy chain and light chain variable regions as described below. A combination of some of these approaches was used for each antibody.

Light chain batch shuffle: Heavy chains from the naïve output were used to prepare light chain diversification libraries. Selections were performed on these libraries as described above, i.e., with one round of MACS and four rounds of FACS. In the different FACS selection rounds, the libraries were evaluated for, e.g., PSR binding and affinity pressure by antigen titration. Sorting was performed to obtain a population with the desired characteristics. Individual colonies from each terminal FACS selection round were picked for sequencing and characterization.

CDRH1 and CDRH2 selection: The CDRH3 of a single antibody was recombined into a premade library with CDRH1 and CDRH2 variants of a diversity of ~-1E8 and selections were performed with one round of MACS and four rounds of FACS as described in the naïve selections. For each FACS round the libraries were looked at for PSR binding and affinity pressure, and sorting was performed to obtain a population with the desired characteristics.

CDRH3 selection: Oligos were ordered which comprised the CDRH3 as well as a flanking region on either side of the CDRH3. Each oligo variegated one or two amino acids in the CDRH3 via NNK diversity. The CDRH3 oligos were recombined with heavy chain FR1-FR3 variable regions containing selected variants from the CDRH1 and CDRH2 selections, and then recombined with the light chain from the parental antibody, for a combined library diversity of ~1E5. Selections were performed with one round of MACS and three rounds of FACS as described in the naïve selections. For each FACS round the libraries were looked at for PSR binding and affinity pressure, and sorting was performed to obtain a population with the desired characteristics. For these selections affinity pressures were applied by preincubating the antigen with parental IgG for 30 minutes and then applying that precomplexed mixture to the yeast library for a length of time which would allow the selection to reach an equilibrium. The higher affinity antibodies were then able to be sorted.

### Antibody Production and Purification

Yeast clones were grown to saturation and then induced for 48 h at 30° C. with shaking. After induction, yeast cells were pelleted and the supernatants were harvested for purification. IgGs were purified using a Protein A column and eluted with acetic acid, pH 3.5.

### KD Measurements

Affinity measurements were performed on an Octet@HTX bio-layer interferometry (BLI) instrument (ForteBio) generally as previously described (see, e.g., Estep et al, Mabs 5(2), 270-278 (2013)). Briefly, affinity measurements were performed by loading IgGs on-line onto AHC sensors. Sensors were equilibrated off-line in assay buffer for 30 min and then monitored on-line for 60 seconds for baseline establishment. Sensors with loaded IgGs were exposed to 100 nM antigen for 3 minutes, and afterwards were transferred to assay buffer for 3 min for off-rate measurement. All kinetics were analyzed using the 1:1 binding model.

Kinetic analysis was conducted at 25° C. in a HBS-EP+ running buffer system (10 mM HEPES pH 7.4, 150 mM NaCl, 3 mM EDTA, 0.05% Surfactant P20) using a Biacore 8K optical biosensor (Global Life Sciences Solutions USA, Marlborough, MA). The sample compartment was maintained at 10° C. for the duration of each experiment.

For antibody capture experiments, a goat anti-human Fe antibody (Jackson ImmunoResearch) was covalently coupled to flow cells 1 and 2 of a CM5 sensor chip surface via standard amine coupling (1:1 EDC:NHS) and then blocked with ethanolamine (1.0 M, pH 8.5). The antibodies (10.0 nM in running buffer) were injected (40 s at 10 uL/min) over flow cell 2. A series of concentrations of IL-4 his monomer ranging from 27.0 to 0.111 nM (3-fold dilutions in running buffer) were injected (300 s at 30 uL/min) over flow cells 1 and 2. Dissociation of IL-4 his monomer was monitored for 3600 s. Several blank buffer samples were injected (300 s at 30 uL/min) over flow cells 1 and 2 and used for reference surface subtraction. All surfaces (flow cells 1 and 2) were regenerated via two injections (20 s at 30 uL/min) of 10 mM glycine, pH 1.5.

### Epitope Binning

Epitope binning was performed using a standard sandwich format cross-blocking assay. Control anti-target IgG was loaded onto AHQ sensors and unoccupied Fc-binding sites on the sensor were blocked with an irrelevant human IgG1 antibody. The sensors were then exposed to 100 nM human IL-4 his antigen followed by a second anti-IL-4 antibody. Additional binding by the second antibody after antigen association indicates an unoccupied epitope (non-competitor), while no binding indicates epitope blocking (competitor).

### Size Exclusion Chromatography (SEC)

A TSKgel SuperSW mAb high throughput (HTP) SEC column (22855) was used for fast SEC analysis of mammalian produced mAbs at 0.4 mL/min with a cycle time of 6 min/run. 200 mM Sodium Phosphate and 250 mM Sodium Chloride was used as the mobile phase.

Dynamic scanning fluorimetry

10 μL of 20× Sypro Orange was added to 20 μL of 0.2-1 mg/mL mAb or Fab solution. A real-time polymerase chain reaction (RT-PCR) instrument (BioRad® CFX96 RT PCR) was used to ramp the sample plate temperature from 40 to 95° C. at 0.5° C. increment, with 2 min equilibrate at each temperature. The negative of the first derivative for the raw data is used to extract Tm.

### Generation of Anti-IL-13 Antigen Binding Domains

### Mouse Immunization and Screening

New binders against IL-13 were generated by immunization of Human immunoglobulin (Ig) transgenic mice (HHKK) and by phage display as known in the art. Affinity was improved using a yeast display method described below.

### Yeast Display Library for Affinity Improvement

Linear DNA encoding a site-saturation library with single amino acid mutations on each VH position of clone CL-203143 was synthesized amplified by PCR. Preparation of competent yeast cells EBY100 (ATCC® MYA-4941) and library transformation was performed. Libraries were generated by gap repair cloning in yeast cells electroporated with the digested vector coding for the VH-C_{H1}-Aga2 fusion and the library PCR product, together with the vector coding for the CL-203143 light chain. Transformed cells were plated on 20×20 cm agar plates containing selective medium (Yeast Nitrogen Base w/o amino acids 6.7 g/l (YNB), Glucose 20 g/l, complete supplement mixture 0.64 g/l (CSM) without the tryptophan and leucine amino acids (WL), ampicillin 100 mg/l, kanamycin 50 mg/l) and incubated for 2-3 days at 28° C. After incubation, cells were scraped from the plates. The harvested yeast cells were resuspended in storage medium (Bacto Yeast Extract 10 g/L+ Bacto Peptone 20 g/L (YP), 30% glycerol) and aliquoted for long-term storage.

### Flow Cytometry Sorting of Yeast Cells

A frozen aliquot of the yeast library was thawed and cultured for 24 h in selective medium at 28° C. with 180 rpm shaking. Cells were diluted to an OD600 of 1 in induction medium (YNB, Galactose 20 g/L, CSM w/o WL, Na₂HPO₄ 5.4 g/L, NaH₂PO₄ 8.6 g/L, ampicillin 100 mg/l, kanamycin 50 mg/l) and incubated for 24 h at 20° C. with 180 rpm shaking to allow Fab expression. After induction, yeast cells were incubated in PBS-1% BSA containing His-tagged human IL-13; IL-13 concentration was decreased across selection rounds (see slide 6 for details). Cells were incubated with shaking for 2 hours at room temperature. Cells were washed with ice-cold PBS-1% BSA before incubation with anti-His tag/Alexa Fluor 647+ anti-HA tag/DyLight 488-conjugated antibody to detect HC for 1 h on ice. Cells were washed with 1 mL of ice-cold PBS-BSA and sorted with a Tyto cell sorter (Miltenyi Biotech). After sorting, cells were either plated on agar plates to isolate single cells or grown in selective liquid medium for bulk analysis.

Sequencing Constructs from Yeast

Plasmids were extracted from yeast using the Zymoprep Yeast Plasmid Miniprep II kit. The VH region was amplified by PCR. For single cell analysis, the amplified VH was Sanger-sequenced, while the amplified VH was submitted to NGS using an Illumina MiSeq device (2×300 bp) for bulk analysis. NGS data were analyzed by counting identical sequences to estimate the mutant frequency in each population. The mutant enrichment ratio was then calculated by comparing populations across successive rounds.

### IgG Reformatting and Production

DNA sequences for selected V_{H} mutants were synthesized as string DNA fragments and cloned into a eukaryotic expression vector coding for the heavy chain of human IgG1. Corresponding mAbs were produced by transient transfection of human FreeStyle™ HEK293-F cells. After 7 days, culture media were harvested and filtered before IgG purification on protein A columns.

### Affinity Measurement by SPR

Binding kinetics were determined using a Biacore T200 instrument. IgGs were captured on a CM5 sensor chip using anti-huFc capture. Association of human IL-13 or cynomolgus IL-13 was measured at different concentrations (0 to 100 nM) for 300 seconds before dissociation in kinetic buffer (5 min and 30 min for the lowest and highest IL-13 concentrations, respectively). Data were analyzed using the T200 evaluation software. Sensorgrams were double-referenced with a reference flow cell (to remove weak non-specific binding and bulk) and a blank injection (to remove drift). Binding kinetics were fitted using a global 1:1 Langmuir binding model.

### Analytical HIC

All hydrophobic interaction chromatography (HIC) analyses were done on an Agilent 1260 HPLC with a TSKgel Butyl-NPR (ID=4.6 mm, length=3.5 cm, 2.5 μm particle size) (Millipore-Sigma #814947) using a concentration gradient of 1.5 M ammonium sulfate, 25 mM sodium phosphate pH 7.0, and 25 mM sodium phosphate pH 7.0. For each sample, 5 μg of protein was injected over the column at 0.5 mL/min for 20 min and an absorbance at 280 nm was measured. Resulting chromatograms were blank subtracted and manually integrated to determine the percent main peak.

### DSF

Nano Differential Scanning Fluorimetry (nanoDSF) was performed using UNcle screening platform (UNchained Labs, Pleasanton, CA, USA). Intrinsic tyrosine and tryptophan fluorescence were measured during thermal denaturation from 25° C. to 90° C. at 1° C./min ramp.

cIEF

Capillary isoelectric focusing (cIEF) was performed using a Maurice C cIEF instrument (Bio-techne, Minneapolis, MN, USA). Approximately 4 ug of protein was diluted in water to a salt concentration less than 15 mM. The master mix was prepared according to the manufacturer's protocol and the following pI standards, 4.225 & 9.50.

### Capillary Zone Electrophoresis

Multiple reagents (water; matching buffer, i.e. 10 mM Histidine pH 6.0; NaOH; DEAE Dextran solution; and DMSO) were loaded in a specific configuration into a PA800 Plus Pharmaceutical Analysis System (Beckman Coulter), using a bare fused silica capillary (50 μm ID×67 cm) with effective and total lengths of approximately 20 cm and 30 cm, respectively. Samples were diluted to 0.3 mg/mL in MiliQ water, and each sample was run at multiple separation potentials (6, 8, 10, 12, and 14 kV) with reverse polarity, following an 8-step operation sequence that included capillary cleaning, equilibration, and sample loading. The relative migration times of DMSO and sample peaks were recorded at each potential. The sample effective valence (z*) was calculated using the equation z*=(μp·kB·T)/De, where the diffusion coefficient is derived from the hydrodynamic radius using the Stokes-Einstein equation.

kD-DLS

Analysis of kD-DLS (diffusion interaction parameter by dynamic light scattering) was performed using a DynaPro™ Plate Reader II (Wyatt Technology/Waters Corp., Milford, MA) dynamic light scattering instrument. The antibodies were dialyzed into 10 mM histidine pH 6.0 and concentrated to 5 mg/mL. Dilutions of the stock solution from 4.5-1 mg/mL in 0.5 mg/mL increments were made in triplicate. The samples were vacuum filtered using a 0.45 um filter plate (MilliPore). The filtrate was collected in a UV-STAR® 384-well plate (Greiner Bio-One North America, Monroe, NC). The apparent diffusion coefficient (Dₐₚₚ) was measured in each well at 20° C. and the concentration was measured using a StUNner UV-Vis and dynamic light scattering instrument (UNchained Labs, Pleasanton, CA). DO is the apparent diffusion coefficient at 0 mg/mL. Each Dₐₚₚ was plotted as a function of concentration and a linear fit was performed to determine the diffusion interaction parameter (kD; in mL/g).

### A280

Analysis of A280 was completed using a StUNner UV-Vis (UNchained Labs, Pleasanton, CA). 3 μL of proteins were loaded into a microfluidic plate and read on the StUNner using Protein (Turbidity)+Sizing. The resulting A280 values were corrected using the extinction coefficients to give the concentration of the proteins. Binding kinetics were determined using a Biacore T200 instrument. IgGs were captured on a CM5 sensor chip using anti-huFc capture. Association of human IL-13 or cynomolgus IL-13 was measured at different concentrations (0 to 100 nM) for 300 seconds before dissociation in kinetic buffer (5 mn and 30 min for the lowest and highest IL-13 concentrations, respectively). Data were analyzed using the T200 evaluation software. Sensorgrams were double-referenced with a reference flow cell (to remove weak non-specific binding and bulk) and a blank injection (to remove drift). Binding kinetics were fitted using a global 1:1 Langmuir binding model.

### Bispecific CODV Antibodies Targeting IL-4 and IL-13

### Expression and Purification of Bispecific CODVs

The fully automated mammalian cell secretory overexpression system, Protein Expression and Purification Platform (PEPP; GNF, San Diego, CA, USA), was used to express and to purify the CODV antibody molecules. In brief, 35 mL of cultures seeded with 3.2×106 Expi293F cells (Thermo Fisher Scientific A14527, Waltham, MA, USA) were transfected with 34 μg of plasmid DNA using the Expifectamine 293 transfection reagent per the manufacturer's protocol (Life Technologies Corporation, Carlsbad, CA, USA). Cells were incubated at 37° C. in an 8% CO2 gas environment for 5 days. Culture supernatants were harvested and applied to MabSelect SuRe protein A resin (GE Healthcare 17-5438-02, Chicago, IL, USA) for gravity flow purification. Proteins were desalted using Nap-10 sephadex (GE Healthcare 17-0854-02) and eluted with 1×DPBS (Thermo Fisher Scientific 14190136), resulting in a final storage solution of phosphate buffered saline, pH 7.4 for each protein sample. The titer was determined using the final concentration and volume and dividing by initial culture size (35 mL).

### Analytical SEC

Samples were measured on a Waters Acquity Ultra-Performance Liquid Chromatography system (UPLC) with a Superdex 200 Increase 5/150 GL column (Cytiva) at 0.3 mL/min with PBS pH 7.2 mobile phase. For each sample, 10 μg of protein was injected in singlet over the column at 0.3 mL/min for 15 min and an absorbance at 280 nm (A280) was measured. Resulting chromatograms were blank subtracted and manually integrated to determine percent purity.

### Affinity Measurement by SPR

The binding profiles of IL-4xIL-13 CODV antibodies to human and cynomolgus monkey IL-4 and IL-13 were evaluated by surface plasmon resonance (SPR), and affinities were compared to comparator trispecific antibodies. The binding of the identified antigen binding domains was assessed as follows. The kinetics of IL-4 and IL-13 binding to the IL-4xIL-13 CODV Fab for the comparator and designed molecules was assessed by surface plasmon resonance (SPR) on a Biacore 8K+ instrument (Cytiva). Anti-human IgG specific for Fab regions (Millipore Sigma) was diluted to 50 ug/mL in 10 mM sodium acetate pH 4.5 and directly coupled to the surface of a CM5 Series S sensor chip via amine coupling (Cytiva) to an immobilization level of >10,000 RU. The running buffer was HEPES buffered saline with 3 mM EDTA and 0.05% tween-20 (HBS-EP+), unless otherwise specified. The trispecific antibodies were diluted to 5 ug/mL and injected over the sensor surface for 60 sec at 10 uL/min to a capture level of ~125-200 RU. The IL-4 and IL-13 antigens were serial diluted 4- and 3-fold, respectively, starting from 5 and 33.33 nM to final concentrations of 19.5 and 45.7 pM. The association time for both antigens was 300 sec, while the dissociation times were specific to each antigen. For IL-4, the 5 nM antigen injection had a dissociation time of 2400 sec while all other concentrations were performed at 120 sec. All IL-13 antigen injections were performed with a dissociation time of 900 sec. The surface was regenerated with two injections of 0.85% phosphoric acid for 60 sec at 30 uL/min for subsequent injections. The data was imported into the BiaEvaluation software and all concentration-dependent sensorgrams were modeled to a 1:1 binding model to determine the association rate (ka, 1/Ms), dissociation rate (kd, 1/s), dissociation constant (KD, M).

### Results

### Generation of Monospecific IL-4 and IL-13 Binders

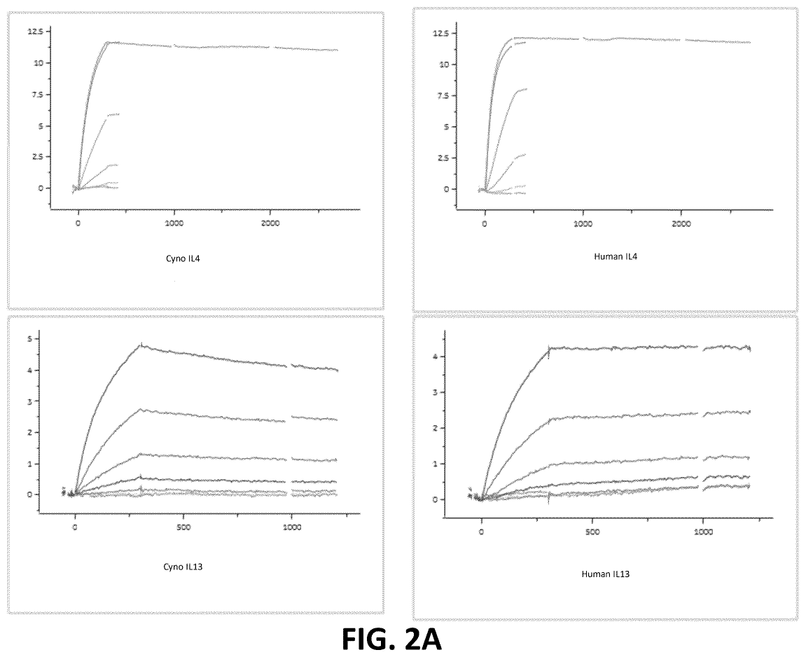

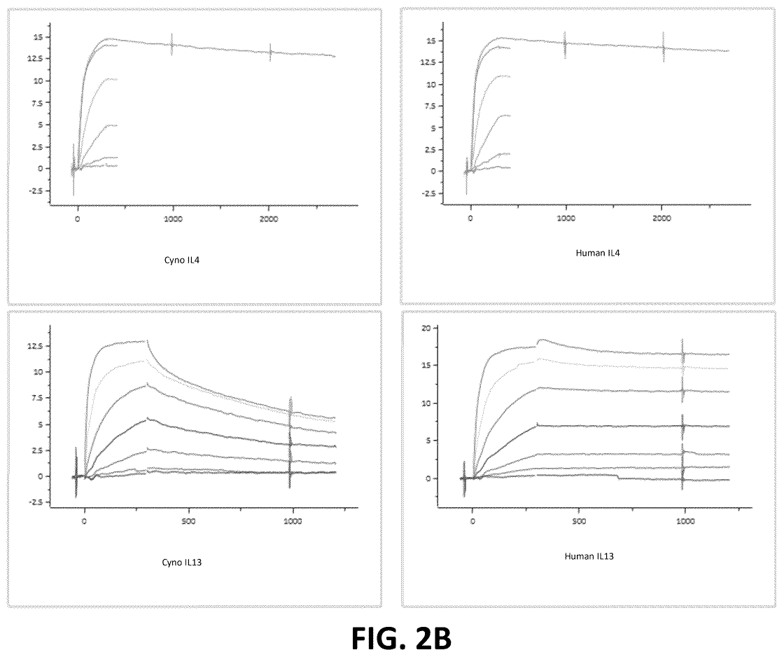

The experiments described above allowed the identification of antigen binding domains with favorable binding characteristics. Favorable antigen binding domains with favorable binding characteristics for human as well as cyno IL-4 include ADI-46653 (VH and VL domains comprising SEQ ID Nos:56 and 43, respectively) and ADI-46659 (VH and VL domains comprising SEQ ID Nos:45 and 43, respectively) (FIG. 2A). Antigen binding domains with favorable binding characteristics for human as well as cyno IL-13 include CL-203143 (VH and V_{L} domains comprising SEQ ID Nos:76 and 77, respectively) and E4 (FIG. 2A). Tables 5 and 6 below indicate the determined kD values for the identified antigen binding domains as well as for comparator molecules (see also FIG. 2B).

TABLE 5
Binding profiles of ADI-46659 and CL-203143 antigen binding domains
to human/cyno IL-4 and human/cyno IL-13, respectively.

| Antigen | On rate (1/ms) | Off rate (1/s) | KD (M) |
|---|---|---|---|
| Cyno IL-4 | 2.76E+06 | 5.72E−05 | 2.00E−11 |
| Human IL-4 | 4.37E+06 | 7.24E−06 | 1.70E−12 |
| Cyno IL-13 | 1.90E+06 | 3.23E−04 | 1.70E−10 |
| Human IL-13 | 1.15E+06 | 1.33E−04 | 1.16E−10 |

TABLE 6
Binding profiles of antigen binding domains from respective
comparator molecules to human/cyno IL-4 and human/cyno IL-13.

| Antigen | On rate (1/ms) | Off rate (1/s) | KD (M) |
|---|---|---|---|
| Cyno IL-4 | 1.49E+07 | 1.27E−04 | 1.52E−11 |
| Human IL-4 | 4.65E+07 | 1.22E−03 | 2.63E−11 |
| Cyno IL-13 | 1.21E+06 | 2.90E−03 | 2.45E−09 |
| Human IL-13 | 3.31E+05 | 1.01E−03 | 3.06E−09 |

To further optimize the binders, ADI-46653, ADI-46659, and CL-203143, several amino acid exchanges where tested. For ADI-46653, the tested amino acid exchanges comprised D111E (VH domain), D111S (VH domain), and H108K (VH domain), tested individually and in combination. Moreover, the tested amino acid exchanges comprised V12K (VH domain), D28S (V_{L} domain), L54S (V_{L} domain), and T96K (V_{L} domain), collectively labelled as “V2”, tested alone or in combination with the amino acid exchanges from the previous sentence. For ADI-46659, the tested amino acid exchanges comprised D111E (VH domain) and D111S (VH domain). Moreover, the tested amino acid exchanges comprised V12K (VH domain), D28S (V_{L} domain), L54S (V_{L} domain), T96K (V_{L} domain), collectively labelled as “V2”, tested alone or in combination with the amino acid exchanges from the previous sentence. For CL-203143, the tested amino acid exchanges comprised N106Q (VH domain), T107Y (VH domain), M5T (V_{L} domain), and S66K (V_{L} domain), collectively labelled as “V3.4”. Asparagine deamidation liability at HC N106 position was addressed by mutation of Asparagine to Glutamine at HC position 106. Methionine oxidation liability at LC M5 position was addressed by mutation of Methionine to Threonine at LC position 5. O-glycosylation at LC S66 position was addressed by mutation of Serine to Lysine at LC position 66. All tested amino acid exchanges mentioned in this paragraph yielded functional proteins, some improved properties of the molecule like stability and viscosity (see also developability testing of the trispecific molecules below). Affinity to huIL13 was increased 100-fold by mutation of Threonine to Tyrosine at HC position 107.

### Bispecific CODV Antibodies Targeting IL-4 and IL-13

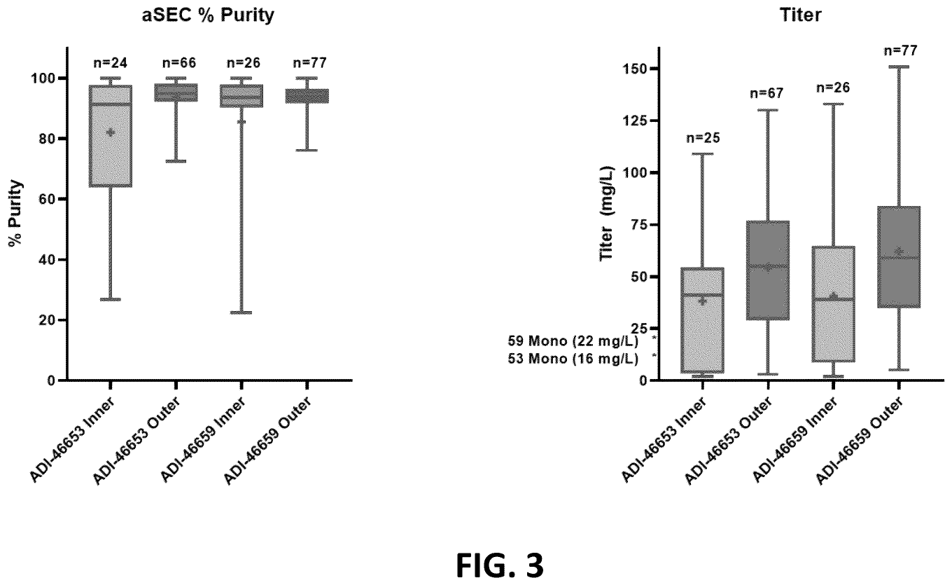

When those favorable antigen binding domains were combined in a CODV bispecific antibody (see FIG. 1), it was surprisingly found that placing the anti-IL-4 antigen binding domain in the outer position (VH most N-terminal on the heavy chain) of the CODV arm was more favorable. Having the anti-IL-4 antigen binding domain in the outer position, and thus the anti-IL-13 antigen binding domain in the inner position, led to much more molecules which could be expressed than the other orientation and moreover improved titer and purity (FIG. 3).

### Example 2: Development and Characterization of Trispecific CODV Antibodies Targeting IL-4 and IL-13

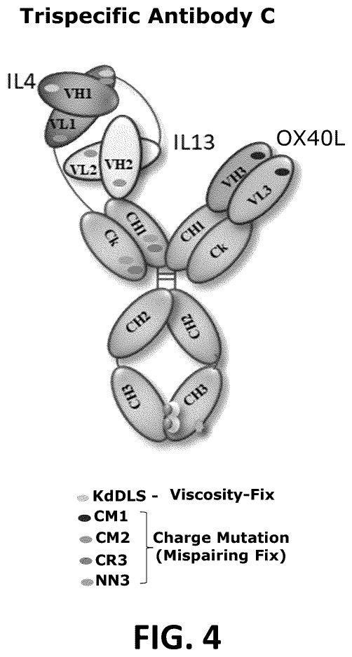

Trivalent, trispecific antibodies that include one Fab arm and one CODV arm with an anti-IL-4 antigen binding domain and an anti-IL-13 antigen binding domain were generated (FIG. 4). The Fab arm can be engineered to target a variety of antigens, such as OX40L, as shown in FIG. 4. The generation of OX40L Fabs is described in Example 3 below.

To optimize the properties of the antibody or decrease chain mispairing, additional mutations can be introduced which can include charge mutations (CM1, CM2, CR3, and NN3), mutations improving molecular properties such as viscosity by Kd-DLS (V2+D111S), LALA mutations, DQ mutations, knob-in-hole mutations, and RF mutations, e.g., to aid in purification or proper chain pairing.

### Materials and Methods

### Production and Characterization of Antibodies

In this example, trispecific antibodies A, B, C, and E were used. All of these antibodies have a similar structure, with one CODV arm and one Fab arm as shown in FIG. 4. All antibodies had similar human IgG1 Fc regions with knob-in-hole and other mutations, IL-4 antigen binding domain on the outer part of the CODV arm, IL-13 antigen binding domain on the inner part of the CODV arm, and OX40L antigen binding domain on the Fab arm. As shown in Table 7, trispecific antibodies B and C were derived from parental antibody A, but included different sets of charge mutations in the V_{H} and V_{L} domains of each of the 3 antigen binding domains to promote proper chain pairing. Trispecific antibody E used a different parental anti-IL-4 binding domain as trispecific antibodies A, B, and C. Trispecific antibody C comprises 1^{st}, 2^{nd}, 3^{rd} and 4^{th} polypeptide chains comprising SEQ ID Nos:98-101, respectively.

TABLE 7
Antigen binding domains for selected trispecific
antibodies (numbers refer to SEQ ID numbers).

| | | |
|---|---|---|
| Anti-IL-4 | Anti-IL-13 | Anti-OX40L |

| Trispec. Ab | VH | VL | VH | VL | VH | VL |
|---|---|---|---|---|---|---|
| A | 42 | 43 | 74 | 75 | 95 | 96 |
| B | 36 | 37 | 72 | 73 | 93 | 94 |
| C | 34 | 35 | 70 | 71 | 91 | 92 |
| E | 54 | 43 | 74 | 75 | 95 | 96 |

### In Vitro Efficacy Assays

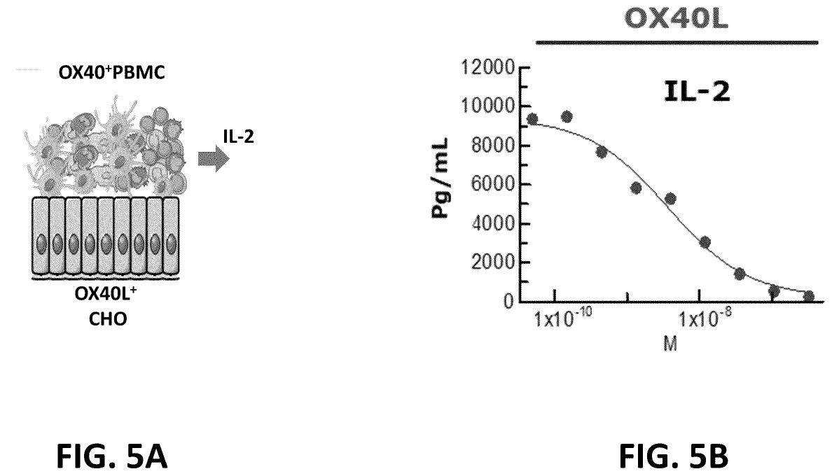

The ability/potency of the anti-OX40L arm of the trispecific antibody to block IL-2 secretion upon OX40L/OX40 interaction was assessed in the setup shown in FIG. 5A. Peripheral blood mononuclear cells (PBMCs) isolated from human whole blood by Ficoll were activated with suboptimal concentrations of phytohemagglutinin (PHA). Then the PBMCs were co-cultivated with x-ray treated (50Gy, 11 min 34 sec) stable human OX40L-expressing CHO cells (CHO-OX40L) in the presence or absence of varying concentrations of the trispecific antibody (targeting IL-4, IL-13 and OX40L). The levels of IL-2 were measured after the cells were cultured at 37° C. for 3 days. After 3 days of co-culture, IL-2 levels in the supernatant were determined using the hIL-2 HTRF kit from Cisbio according to manufacturer's instructions. Measurements were performed in a PheraSTAR FSX (BMG) equipped with an HTRF laser and optics modules.

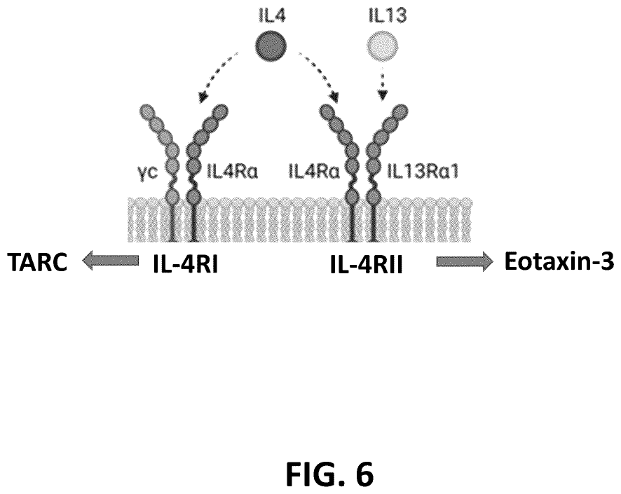

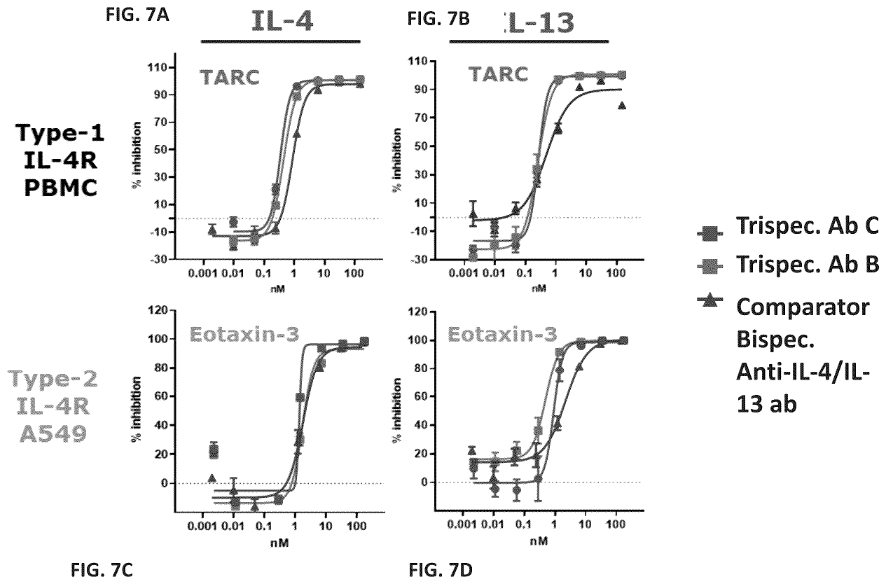

The ability/potency of the anti-IL-4/IL-13 arm of the trispecific antibody to block thymus and activation-regulated chemokine (TARC, also known as CCL17) and eotaxin-3 (also known as CCL26) secretion upon IL-4 or IL-13 stimulation (see FIG. 6) was assessed in the assay described in the following. IL-4RI⁺ Human PBMCs (FIGS. 7A & 7B) or IL-4RII*human lung fibroblast cell line A549 (FIGS. 7C & 7D) were stimulated with 6 ng/mL of IL-4 (FIGS. 7A & 7C) or 10 ng/mL of IL-13 (FIGS. 7B & 7D) in the presence or absence of indicated antibody for 24 h (FIGS. 7A & 7B) or 48 h (FIGS. 7C & 7D). Supernatants were collected, and the amounts of secreted TARC or eotaxin-3 in the supernatant were measured using ELISA with chemoluminescence detection (MesoScale Discovery).

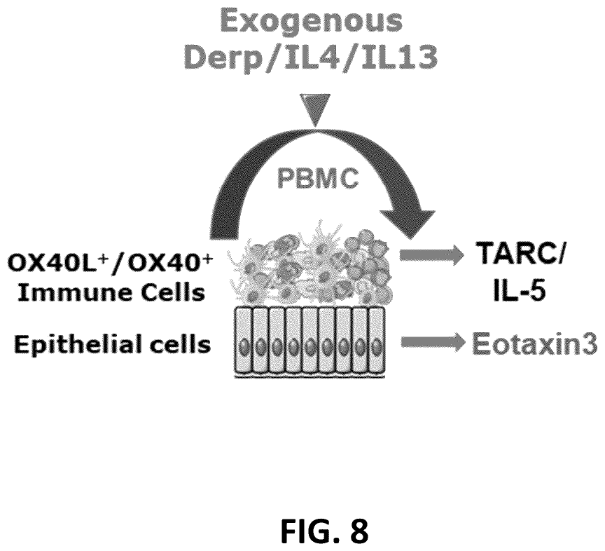

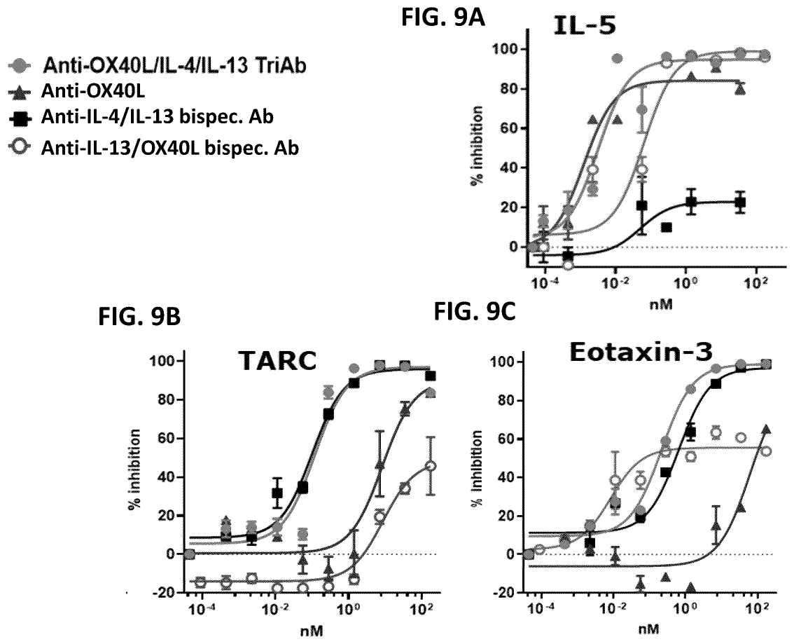

To further assess the ability of the trispecific IL-4/IL-13/OX40L antibodies to block simultaneously effects mediated by IL-4, IL-13 and OX40L, a tissue-mimic assay against the Dermatophagoides pteronyssinus (Der p) allergen was used. Dermatophagoides pteronyssinus is a house dust mite species whose allergen induces chemokine production. In the setup shown in FIG. 8, exogenous Der p, IL-4 and IL-13 were added to a co-culture of OX40L+/OX40⁺ PBMCs and epithelial cells. To this end, human PBMCs (0.8×10≢cells per well) were seeded into 96-well plate which were plated with A549 cells (0.5×10 \hat{ }6 /per well) overnight. Co-culture PBMC and A549 cells were stimulated with the Der P allergen (3 ug/ml) isolated from house dust mites plus IL-4 (6 ng/mL) and IL-13 (10 ng/mL) for 5 days, in presence of the antibodies indicated in FIG. 9. Supernatants were collected and amounts of secreted IL-5, TARC or eotaxin-3 in the supernatant were measured using ELISA with chemoluminescence detection (MesoScale Discovery).

### In Vivo Efficacy Assays

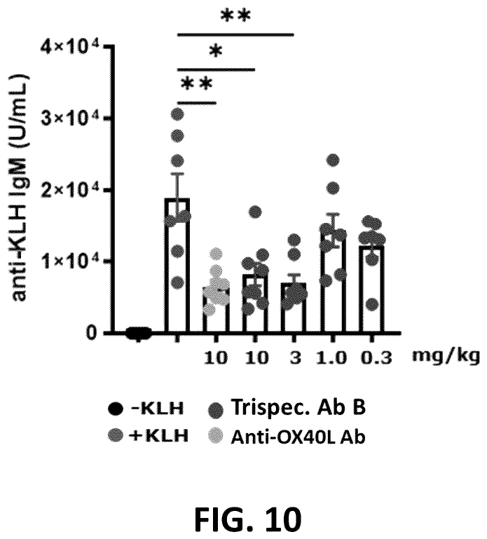

To determine the efficacy of the trispecific anti-IL-4/IL-13/OX40L antibodies in vivo, a mouse model was employed to detect T-cell dependent antibody response (TDAR). Mice were genetically modified to carry human OX40L and OX40 genes to replace mouse orthologs. hOX40 x hOX40 knock-in mice were challenged with saline alone or with the hapten keyhole limpet hemocyanin (KLH) (300 pg/100 μl) and treated with antibodies shown in FIG. 10 at indicated dose on day 0 and day 14 via intraperitoneal route. Mice are bled on day 7 and 21, and euthanized on day 21 to obtain a samples for serum anti-KLH Immunoglobulin (IgG & IgM) analysis. Serum anti-KLH IgG and IgM were measured using the KLH IgG (Mouse) ELISA KIT and KLH IgM (Mouse) ELISA KIT from Abnova on a Molecular Devices SPRECTRAmax 340PC plate reader.

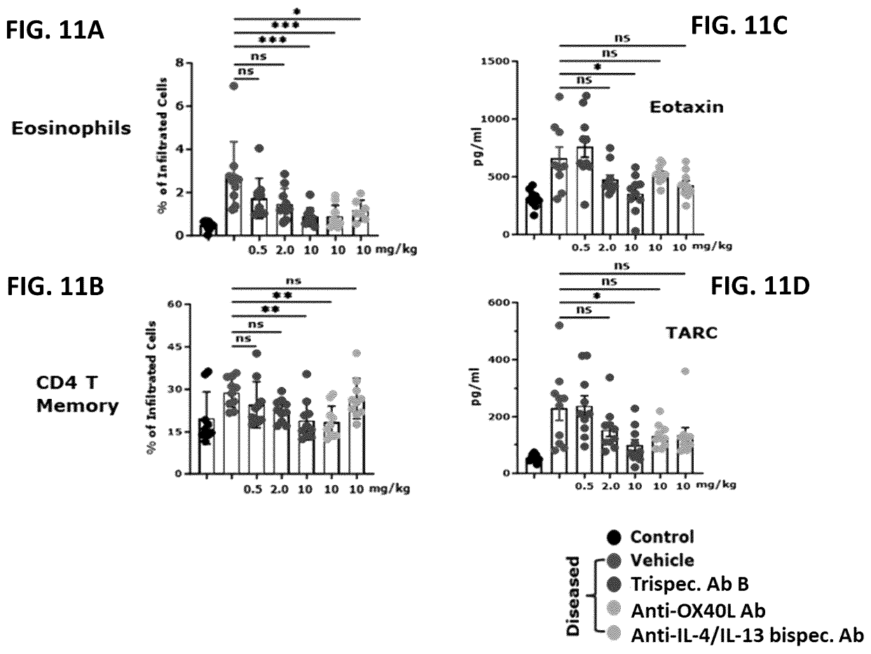

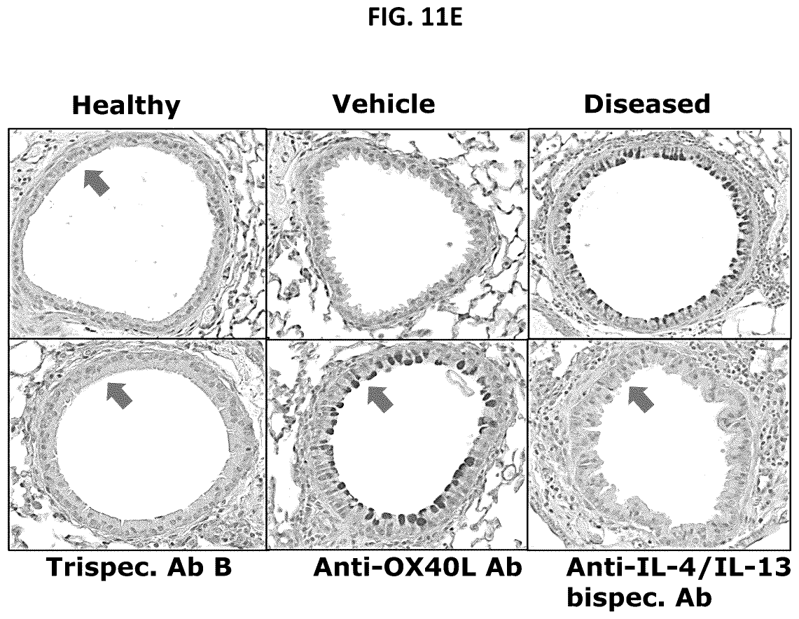

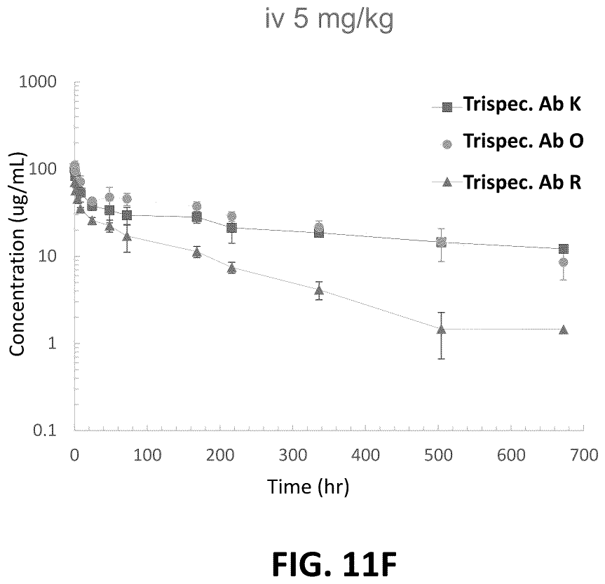

In another mouse model, the effect of the trispecific anti-IL-4/IL-13/OX40L antibodies on the pulmonary immune response was studied. Mice were genetically modified to carry human IL-4, IL-13, IL-4Ra, OX40L, and OX40 genes to replace mouse orthologs. These quintuple knock-in mice were sensitized with MC903 (0.75 nmol/10 μl) topically on the ears twice per week for two weeks. Sensitized mice were then challenged intranasally with ovalbumin (10 ug/50 μl) combined with house dust mite (10 pg per dose) or saline only as control group every other day for two weeks. After 5 days of rest, mice were rechallenged intranasally with ovalbumin (10 ug/50 μl) or saline for three consecutive days before termination. Diseased mice received indicated antibody via intraperitoneal route at a dose of 10 mg/kg on days 14, 17, 21, 24, and 28 of the study. The mice lung were harvested, processed, and analyzed for frequency of eosinophils (FIG. 11A) and CD4 memory T cells (FIG. 11B) by flow cytometry, for levels of eotaxin-3 (FIG. 11C) or TARC (FIG. 11D) using ELISA with chemoluminescence detection (MesoScale Discovery), and for pathological changes using Periodic acid-Schiff (PAS) staining (FIG. 11E).

In a third mouse model, pharmacokinetics (PK) of the trispecific antibodies were analyzed. To this end, trispecific antibodies K, O, and R were administered to hFcRn Tg32 mice (expressing human FCGRT gene). Mice were intravenously treated with a dose of 5 mg/kg of each antibody. The study design is summarized in Table 8.

TABLE 8
Study design for PK assay with trispecific antibodies K, O, and R.

| | | | | | Dose | | |
|---|---|---|---|---|---|---|---|
| | | | Dose | # of | Route/ | | |
| Study | | Protein | (mg/ | Tg32 | Dosing | Sample | Bio- |
| No | Compound | format | kg) | mice | Regimen | collection | analysis |
| PK20- | Trispec. Ab | TRI- | 5 | 4 | Iv/1 | 0.083, 1, | LC- |
| 099 | K | CODV- | | | dose | 4, 8, 24, | MS/MS |
| | | (aIL-4- | | | | 48, 72, | |
| | | ADI- | | | | 168, 216, | |
| | | 46653 × | | | | 336, 504, | |
| | | aIL-13-CL- | | | | 672 h | |
| | | 203143) ×× | | | | post-dose | |
| | | aTNFSF4- | | | | | |
| | | E4- | | | | | |
| | | huIgG1- | | | | | |
| | | LALAKIH- | | | | | |
| | | RF_4 | | | | | |
| PK20- | Trispec. Ab | TRI- | 5 | 4 | | | |
| 100 | O | CODV- | | | | | |
| | | (aIL-4- | | | | | |
| | | ADI- | | | | | |
| | | 46659 × | | | | | |
| | | aIL-13-CL- | | | | | |
| | | 203143) × | | | | | |
| | | aTNFSF4- | | | | | |
| | | MM25PL3A2- | | | | | |
| | | huIgG1- | | | | | |
| | | LALA- | | | | | |
| | | KIH-RF_4 | | | | | |
| PK20- | Trispec. Ab | TRI- | 5 | 4 | | | |
| 101 | R | CODV- | | | | | |
| | | (aIL-13- | | | | | |
| | | CL- | | | | | |
| | | 204842 × | | | | | |
| | | aIL-4-CL- | | | | | |
| | | 199320) × | | | | | |
| | | aTNFSF4- | | | | | |
| | | E4- | | | | | |
| | | huIgG1- | | | | | |
| | | LALAKIH- | | | | | |
| | | RF_4 | | | | | |

### Developability Assays

### Isoelectric Point

A sample matrix was prepared composed of 0.35% methyl cellulose, 5% Pharmalyte (3-10), pI markers, and protein sample at 0.35 mg/mL. The prepared samples were analyzed using Protein Simple Maurice (Bio-Techne) imaged isoelectric focusing instrumentation. Pre-focusing was performed at 1500 V for 1 minute, followed by focusing at 3000 V for 5 minutes. The data were analyzed using Compass Software for iCE and the isoelectric point was determined based on the migration of the protein sample relative to the pI markers.

### Dynamic Light Scattering (DLS)

Samples were prepared at 0.5 mg/mL in 10 mMv histidine (pH 6.0) and subsequently filtered through 0.22 m spin filters by centrifugation at 10000×g for 1 minute. The samples were loaded into disposable cuvettes and introduced to the measurement chamber of a Wyatt DynaPro NanoStar light scattering instrument and allowed to equilibrate thermally for 10 minutes at 20° C. The samples were then analyzed by performing 20 acquisitions of 5 seconds each. The hydrodynamic radius and polydispersity index were determined using cumulants analysis.

### Diffusion Interaction Parameter

Samples were prepared in 10 mM histidine (pH 6.0) and diluted from 1.0 to 4.5 mg/ml in 0.5 mg/ml increments and loaded in triplicate into an 384-well 0.45-μm filter plate set into a Greiner Bio-One UV-STAR 384-well plate. The plates were centrifuged for 2 minutes at 2000×g to complete filtration. The apparent diffusion coefficient was measured in each well using a Wyatt DynaPro Plate Reader II dynamic light scattering instrument and data were fit with the cumulants model. The absorbance of each well at 280 nm was determined using the Lunatic from Unchained Labs. The mean apparent diffusion coefficient (in cm2/s) of each triplicate sample was plotted as a function of the mean concentration (in grams per milliliter) and linearly regressed to determine the diffusion interaction parameter (kD; in milliliters per gram).

### Viscosity

Samples were concentrated to −150 mg/mL in 10 mM histidine (pH 6.0) using centrifugal concentrators and subsequently passed through 0.22 μm spin filters by centrifugation at 10000×g for 1 minute. Concentration was confirmed using a SoloVPE attachment (C Technologies) on a Cary 50 spectrophotometer. Solution viscosities were measured at 20° C. with a VROC Initium automatic high-throughput viscometer (RheoSense) using shear rate adapted for each solution viscosity such that the pressure drop was in the optimal range for the pressure sensor array of the microfluidic chip.

### Turbidity

Samples were concentrated to −150 mg/mL in 10 mM histidine (pH 6.0) using centrifugal concentrators and subsequently passed through 0.22 μm spin filters by centrifugation at 10000×g for 1 minute. Concentration was confirmed using a SoloVPE attachment (C Technologies) on a Cary 50 spectrophotometer. Analysis was performed by p-nephelometry using a “de-tuned” static light scattering channel of a Wyatt DynaPro NanoStar laser light scattering instrument. Briefly, samples were loaded in 10-pI quantities into NanoStar disposable cuvettes. The instrument was set to acquire data with the laser power diminished to 1%. The static scattering detector voltage was recorded and calibrated against that of a set of polymer bead turbidity standards of 1, 5, 10, 20, 40, and 80 NTU.

### Size Exclusion Chromatography

Samples were prepared in 10 mM histidine (pH 6.0) at 1 mg/mL. Chromatography was performed using a Waters Acquity UPLC BEH200 4.6×300 mm, 1.7 aμm particles (Waters, ref. 186005226) stationary phase with a mobile phase composed of 50 mM sodium phosphate, 300 mM sodium perchlorate (pH 7.0). Composition of high molecular weight species was determined by integrating all protein peaks and determining the relative area under the curve of apparent high molecular weight species.

### Results

As shown in FIG. 5B, concentration of IL-2 in the supernatant was depleted in a dose-dependent manner with increasing concentration of trispecific antibody C, indicating trispecific antibody C binds OX40L on the CHO cells, preventing OX40L-induced IL-2 production. OX40L-induced IL-2 inhibition was also tested with the trispecific antibody B (same overall structure and antigen binding domains as trispecific antibody C, except different pairs of charge mutations to promote proper chain pairing) and a comparator anti-OX40L monoclonal antibody. IC50 results in Table 9 indicated that the trispecific antibodies had stronger IC50 values than the comparator anti-OX40L antibody.

As shown in FIGS. 7A & 7B, TARC inhibition increased in a dose-dependent manner, and trispecific antibodies C and B had a lower IC50 values than the comparator bispecific (Table 9). To further determine whether the trispecific antibodies can inhibit IL-4 or IL-13-induced Eotaxin-3, IL-4RIIL A549 cells were used in a similar manner as above. As shown in FIGS. 7C & 7D and Table 9, trispecific antibodies C and B showed strong IL-4 and IL-13-mediated inhibition.

TABLE 9
IC50 of trispecific antibodies C and B as compared
to a comparator anti-OX40L monospecific antibody and
a comparator bispecific anti-IL-4/IL-13 antibody.

| | IC50 nM | | | | |
|---|---|---|---|---|---|
| | OX40L | IL-4 | | IL-13 | |
| TriAb ID | IL-2 | TARC | Eotaxin-3 | TARC | Eotaxin-3 |
| Trispec. Ab C | 4.46 | 0.31 | 1.19 | 0.22 | 0.87 |
| Trispec. Ab B | 2.90 | 0.78 | 1.63 | 0.20 | 0.63 |
| Anti-OX40L | 8.21 | N/A | N/A | N/A | N/A |
| Anti-IL-4/IL- | N/A | 1.03 | 1.55 | 0.53 | 1.93 |
| 13 bispec. | | | | | |

FIGS. 9A-9C indicate the results of the Der P-dependent tissue mimic assay. Percent inhibition of IL-5, TARC and Eotaxin-3 were measured after adding a titration of trispecific antibody C (anti-OX40L/IL-4/IL-13 trispecific antibody), a comparator anti-OX40L/IL-13 bispecific VHH antibody, a comparator anti-IL-4/IL-13 bispecific antibody, and a comparator anti-OX40L monoclonal antibody. A comparator antibody that binds IL-4Ra, blocking IL-4 and IL-13 binding, was further tested.

Results of the allergen-driven assay show trispecific antibody C demonstrated superior inhibition of IL-5, TARC and Eotaxin-3 (FIGS. 9A-9C, Table 10).

TABLE 10
IC50 of trispecific antibody C as compared to
anti-OX40L/IL-13 bispecific nanobody, anti-OX40L,
bispecific anti-IL-4/IL-13 and anti-IL-4Rα.

Allergic PBMCs

| Antibody (IC50 nM) | TARC | Eotaxin-3 | IL-5 |
|---|---|---|---|
| Trispec. Ab C | 0.13 | 0.11 | 0.08* |
| Bispec. OX40L/IL-13 VHH | 8.72 | 0.01 | 4.00 |
| Anti-OX40L | 17.24 | 22.92 | 0.001 |
| Bispec. Anti-IL-4/IL-13 | 0.09 | 0.14 | N/D |
| Anti-IL-4Rα | 0.14 | 0.02 | N/A |

Next, the affinity of parental trispecific antibody A, and variants B and C were tested for their affinity to human OX40L, human IL-4 and human IL-13, as compared to the comparator antibody anti-IL-4/anti-IL-13/anti-OX40L. IC50 values for an IL-2 release assay (see above) was also compared. As shown in Table 11, all trispecific antibodies A, B, and C showed high affinity to all three antigens, and a lower IC50 value as compared to the comparator antibody. Moreover, trispecific antibody A, B, and C were more potent in the IL-2 release assay.

TABLE 11
Binding affinity to human OX40L, IL-4 and IL-13, and IL-2 release
assay IC50 for trispecific antibodies A, B, C, and comparator.

| | Affinity - KD (M) | | | IL2 release |
|---|---|---|---|---|
| Molecule | Hu OX40L | Hu IL-4 | HU IL-13 | assay IC50 |
| Trispec. Ab C | 1.72E−10 | 2.71E−12 | 7.83E−11 | 6.12E−09 |
| Trispec. Ab B | 2.78E−11 | 3.38E−12 | 1.20E−10 | 5.40E−09 |
| Trispec. Ab A | 6.40E−11 | 5.30E−12 | 4.15E−10 | 3.15E−09 |
| Comparator | 3.21E−09 | 1.62E−11 | 7.48E−10 | 1.23E−08 |
| trispec. Anti-IL- | | | | |
| 4/IL-13/OX40L | | | | |

In the in vivo T-cell dependent antibody response (TDAR) assay, the concentration of anti-KLH IgM) were measured. As shown in FIG. 10, after 7 days, the increasing concentrations of trispecific antibody B (IL-4/IL-13 CODV/OX40L Fab) were able to inhibit the formation of anti-KLH IgG, demonstrating that the trispecific antibody was capable of blocking TDAR.

The inhibition of the pulmonary immune response was studied in a second mouse model. FIG. 11 shows eosinophil numbers (FIG. 11A), CD4 memory T cell number (FIG. 11B), eotaxin-3 levels (FIG. 11C), TARC levels (FIG. 11D) and pathological changes (FIG. 11E). It was apparent that trispecific Ab B could inhibit the immune response at least as effective as the comparator antibodies (anti-OX40L and anti-IL-4/IL-13). Regarding the inhibition of TARC levels (FIG. 11D), the Trispec. Ab B was superior to the two comparator antibodies. Moreover, Trispec. Ab B was able to prevent all visible pathological changes (FIG. 11E).

The pharmacokinetics results of the hFcRn Tg32 mouse model are summarized in Table 12. As shown in FIG. 11F, trispecific antibody R exhibited the highest clearance, at 0.993 mL/hr/kg. Trispecific antibodies K and O demonstrated slower clearance, at 0.223 mL/hr/kg and 0.244 mL/hr/kg, respectively in serum. These results indicate that use of the IL-13 antigen binding domain in the outer part of the CODV arm can lead to PK problems.

TABLE 12
Pharmacokinetics assay results for trispecific antibodies K, O, and R.

| | C₀ | Cₗₐₛₜ | AUCₗₐₛₜ | Tₗₐₛₜ | AUC_{inf} | AUCₑₓₜᵣₐₚ | t_{1/2} | CI | V |
|---|---|---|---|---|---|---|---|---|---|
| ID | (μg/mL) | (μg/mL) | (hr*μg/mL) | (hr) | (hr*μg/mL) | (%) | (hr) | (mL/hr/kg) | (mL/kg) |
| Trispec. | 104 | 12.2 | 14200 | 672 | 22900 | 37.8 | 492 | 0.223 | 153 |
| Ab K | | | | | | | | | |
| Trispec. | 111 | 8.53 | 17100 | 672 | 20800 | 16.6 | 270 | 0.244 | 90.3 |
| Ab O | | | | | | | | | |
| Trispec. | 72.2 | 1.18 | 4870 | 546 | 5080 | 4.05 | 119 | 0.993 | 164 |
| Ab R | | | | | | | | | |

The developability of trispecific antibodies was next evaluated. Trispecific antibodies C, B, A, and E were assessed for their yield, mispairing, stability measured by dynamic light scattering (Kd-DLS), viscosity, stability, and deviceability.

Trispecific antibody C showed the highest protein yield, lowest chain mispairing, highest viscosity, and best stability (Table 13).

TABLE 13
Developability profile for trispecific antibodies C, B, A and E.

| Trispec. | Yield | % | Kd-DLs | Viscosity | Stability, | Stability, | |
|---|---|---|---|---|---|---|---|
| Ab | (mg/L) | Mispairing | (ml/g) | (cPa) | 50 mg/mL | 150 mg/mL | Deviceability |
| C | 80.2 | 10% | 28 | 49.3 | ✓ | ✓ | ✓ |
| B | 27.3 | 40% | 22 | 145 | | | |
| A | 44 | 88% | 12.8 | 71.5 | | | |
| E | 16.1 | 90% | −3.2 | 36.7 | | | |

The individual developability characteristics of the trispecific antibodies E, B, and C were further assessed in the following assays.

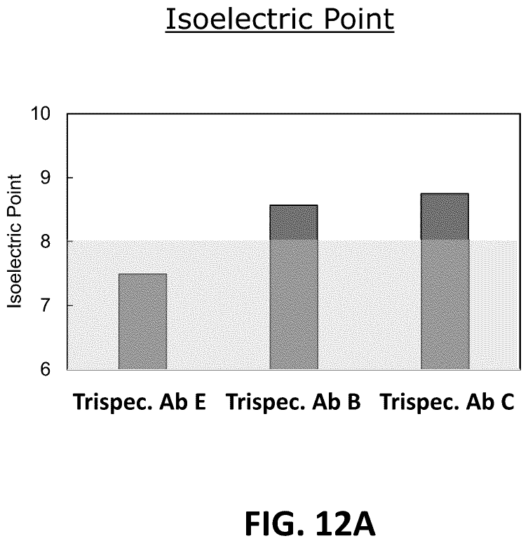

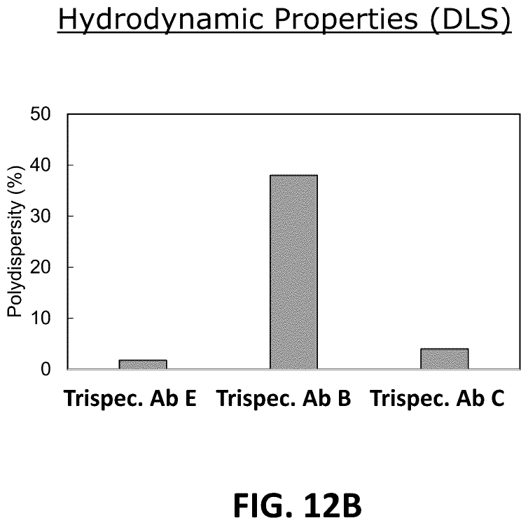

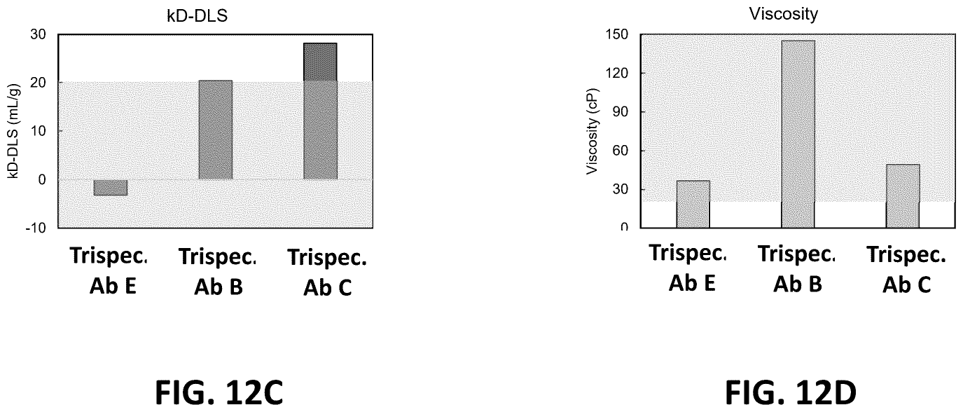

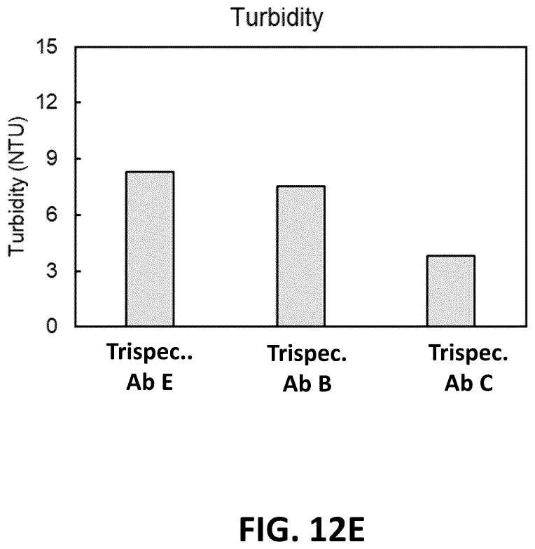

An isoelectric point greater than 8 is desirable for antibody-based biologics. Trispecific antibodies B and C showed an ideal isoelectric point (FIG. 12A).

DLS was performed to evaluate the hydrodynamic properties of trispecific antibodies E, B, and C. Trispecific antibody B showed elevated polydispersity after filtration, suggesting the presence or formation of soluble aggregates, while trispecific antibodies E and C showed low polydispersity (FIG. 12B).

Diffusion interaction parameter (kD) measured by DLS was performed to evaluate colloidal stability. kD-DLS values less than 20 mL/g present a risk for poor colloidal properties, including aggregation, opalescence, and viscosity. As shown in FIG. 12C, trispecific antibody C showed an ideal profile.

Viscosity was also measured. A viscosity less than 20 centipoise (cP) is ideal for antibody therapeutics. Trispecific antibodies E, B, and C were above this threshold, but trispecific antibodies E and C showed the lowest viscosity (FIG. 12D).

Lastly, trispecific antibodies E, B, and C all had turbidity values below the threshold of 12 NTU, with trispecific antibody C having the lowest value (FIG. 12E). These data demonstrate the unfavorable charge mutation orientation on the Fab arm has negative consequences on viscosity and colloidal stability. A summary of these results is shown in Table 14.

TABLE 14
Molecular profiling summary for trispecific antibodies E, B, and C.

| | | Trispec. | Trispec. | Trispec. |
|---|---|---|---|---|
| Stress | Assessment | Ab E | Ab B | Ab C |
| High | Concentration (mg/mL) | 145.8 | 158.0 | 153.7 |
| Concentration | Viscosity (cPa) | 36.7 | 145 | 49.3 |
| Behavior | μ-nephelometry (NTU) | 8.3 | 7.5 | 3.8 |
| | Visual Inspection | Clear | Yellowish | Clear |
| Predictive | kD-DLS (mL/g, H6.0) | −3.2 | 20.4 | 28.2 |
| Testing | | | | |

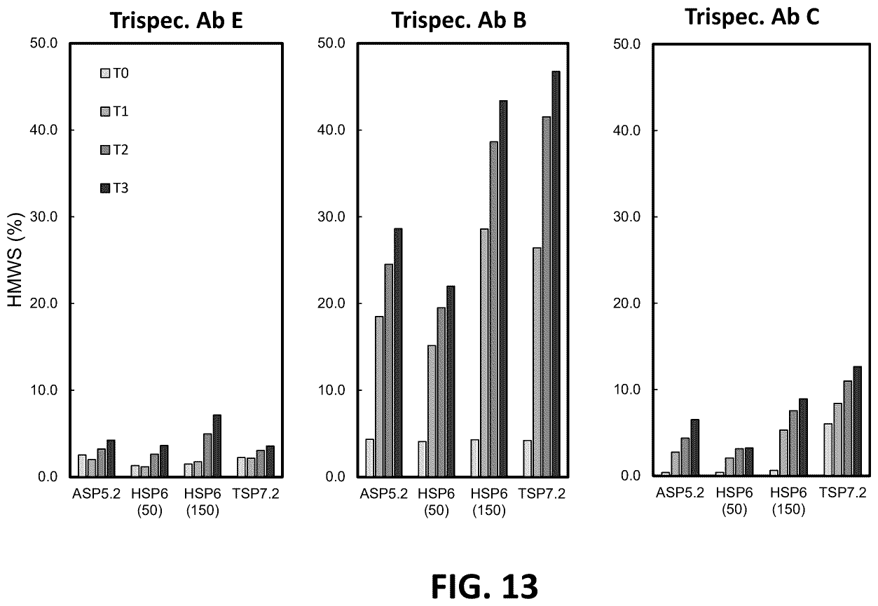

To assess the stability of trispecific antibodies, B, and C, the trispecific antibodies were stored for 3 weeks at 40° C., and the antibodies were evaluated by size-exclusion chromatography to assess aggregation. As shown in FIG. 13, trispecific antibody B had an unfavorable aggregation profile, with close to 50% high molecular weight species in some samples. Trispecific antibody F and C displayed low aggregation, as shown in FIG. 13.

A summary of percentage of low molecular weight and high molecular weight species for trispecific antibodies in size exclusion chromatography samples is summarized in Table 15.

TABLE 15
Size exclusion chromatography data for
trispecific antibodies B, F, and C.

| Trispecific | | | | |
|---|---|---|---|---|
| Antibody | Pool | % HMWS | % LMWS | % Monomer |
| B | A1 | 13.41 | 0 | 86.59 |
| | A2 | 8.23 | 0 | 91.77 |
| | A3 | 2.35 | 0 | 97.65 |
| | A4 | 3.45 | 0 | 96.55 |
| | A6 | 3.64 | 0 | 96.36 |
| | A7 | 9.4 | 0 | 90.6 |
| | A8 | 6.65 | 0 | 93.35 |
| F | B1 | 31.18 | 0 | 68.82 |
| | B2 | 6.7 | 0 | 93.3 |
| | B3 | 12.73 | 0 | 87.27 |
| | B4 | 12.18 | 0 | 87.82 |
| | B5 | No Signal | No Signal | No Signal |
| | B6 | 26.33 | 0 | 73.67 |
| | B7 | 10.05 | 0 | 89.95 |
| | B8 | 39.42 | 0 | 60.58 |
| C | C1 | 2.16 | 1.87 | 95.97 |
| | C2 | 2.46 | 1.78 | 95.75 |
| | C3 | 2.04 | 1.65 | 96.31 |
| | C4 | 1.59 | 1.73 | 96.68 |
| | C5 | 2.01 | 1.82 | 96.17 |
| | C6 | 1.02 | 1.69 | 97.29 |
| | C7 | 1.48 | 2 | 96.52 |
| | C8 | 1.28 | 1.62 | 97.1 |

### Example 3: Generation and Characterization of an OX40L Binder

This Example describes how the OX40L binder MM25PL3A2 (used in Example 2 above in trispecific binding molecules) was generated. Moreover, this Example describes the CryoEM structure of OX40L in complex with MM25PL3A2 Fab deciphered at a resolution of 3.94 Å, which displays a 3:3 binding stoichiometry. The results indicate that three MM25PL3A2 Fabs intercalate between two OX40L trimers partially overlapping OX40 receptor binding site, therefore sterically hindering the binding of the receptor on OX40L.

### Methods

### Generation of MVJ25PL3A2

### Mice and Immunization

Human immunoglobulin (Ig) transgenic mice (HHKK), were intraperitoneally immunized with 100 ug of recombinant human OX40L protein (HuOX40L) five times with a two-week interval time. The mice were finally boosted with 50 ug of HuOX40L four days before harvesting spleen samples for processing.

Fluorescence-Activated Sorting of huOX40L-Specific B Cells

The mice were sacrificed at day four post final boost and splenocytes were harvested. Total B-cells from the single cell suspension of splenocytes were enriched with EasySep™ Mouse Pan-B-cell Isolation Kit (STEMCELL Technologies) following the manufacturer's instructions. The cells were then stained with of fluorochromes conjugated HuOX40L (HuOX40L-AF488, HuOX40L-AF647) and irrelevant antigen (iAg-AF700), as well as B-cell antibody cocktail. HuOX40L specific B-cells were identified as 7AAD-CD19⁺ IgM⁻IgD⁻IgG⁺ Igλ-iAg-AF700⁻HuOX40L-AF488* HuOX40L-AF647⁺. HuOX40L specific B-ells were individually sorted into the wells of a 96-well PCR plate containing 6 μL of a RT reaction mixture.

Isolation of Ig Heavy and Light Chain Variable Genes from Sorted Single B Cells

In the 96-well PCR plate, Ig VH and VL genes were amplified from single B-cells by reverse transcription reaction (RT) and nested polymerase chain reaction (PCR) based on a method previously described previously (Liao et al., J Virol Methods. 2009 June; 158(1-2):171-9). Pooled primers were used for two rounds of PCR reactions. PCR products were puriied using the enzymatic method with ExoSAP-IT (Applied Biosystems) according to manufacturer's instructions.

### Construction of Linear Expression Cassettes (LECs)

Heavy and light chain linear expression cassettes (H_LECs/L_LECs) were constructed based on plasmid templates. The DNA fragment composed of CMV promoter and Ig leader sequence (PCMV fragment, used for both H-LEC and L-LEC), the DNA fragment containing mouse IgG1 constant region, stop codon and rabbit beta globin (rbGlob) poly (A) tail (muIgG1_CH fragment), and the DNA fragment of mouse Igkappa constant region and rbGlob poly (A) tail (mulgCk fragment) were amplified from plasmid templates. The DNA fragments were purified by gel electrophoresis and QIAquick Gel Extraction Kit (Qiagen).

The cleaned amplicons of Ig VH and VL from single B-cells and purified DNA fragments were used for constructing heavy chain and light chain linear expression cassettes by overlapping PCR following the method previously described (Liao et al., J Virol Methods. 2009 Jun; 158(1-2):171-9) H-LEC and L-LEC amplicon generations were purified with MinElute 96 UF PCR Purification Kit and were ready for the transfection to generate small scale of recombinant monoclonal antibodies (mAbs).

### Expression of Recombinant Antibodies

Purified cognate pairs of H_LEC and L_LEC were co-transfected into 0.5 mL of Expi293F cells (Thermo Fisher Scientific) in 96 deep well plate with ExpiFectamine 293 Transfection Kit (Thermo Fisher Scientific) and the cells were incubated at 37° C., 8% CO2 with shaking at 900 rpm. After 5 days, the cell culture supernatants were harvested and immediately used for assessment of antibody quantification, binding to antigen, and functional screening. The antibody concentrations were measured by Octet (Forte Bio) using anti-murine IgG or anti-human IgG Quantitation Biosensors (Forte Bio, AMQ Biosensors) to capture whole mouse or human IgG and Fabs following the manufacturer's instructions.

### Screening Recombinant Monoclonal Antibodies for Antigen Specificity

The rmAbs in cell culture supernatants were screened by flow cytometry for assessing antigen specificity. HEK 293 cells stably expressing huOX40L were incubated with 50 μL of culture supernatant and then stained with Alexa flour 647 conjugated goat anti mouse IgG (H+L) (Thermo Fisher Scientific). Following a final wash, flow cytometric acquisition was performed using BD FACS Canto II (BD Biosciences) and data analyzed using FlowJo 10.6 software.

### Functional Screening Using High-Throughput Reporter Assay

Promega GloResponse™ NFkB-Luc2P/U20S reporter cells (Promega Corporation) were used for functional screening. Reporter cells were added with the supernatants with 9-point, or 18-point serial dilutions were performed on the Agilent Bravo Assay plates were incubated for 4 h at 37° C., and then equilibrated for 15 minutes at room temperature. The Bio-Glo detection reagent (Bio-Glo Luciferase Assay System, Promega Corporation) was prepared and applied to the assay according to the manufacturer's instructions. Luminescence output was measured on the Envision using the US luminescence aperture and 0.1 s/well readout time. Raw data files from the Envision were imported into Genedata Screener (version 16.0.2, Genedata). Data was normalized and scaled using negative and positive controls as central and scale reference, respectively. Plate-based RZ′ factors were calculated in Screener and plates with RZ′<0.5 were masked and excluded from further analysis. Curve fitting was carried out using the Screener SmartFit algorithm, and valid IC50 or EC50 values were reported as qAC50 values.

### Sequencing and Analysis

Immunoglobulin genes of the heavy and light variable domains (VH and VL) which were isolated from single B-cells were sequenced in both forward and reverse direction by Sanger sequencing method. The contigs were assembled with forward and reverse sequences by using the software of Sequencher 5.4.6. The sequences were trimmed to exclude those that are frame shifted, truncated or have stop codons. The clean sequences were then analyzed to identify V, D and J germline subfamilies for VH and VL genes and clone lineage.

Structure Determination of MM25PL3A2 in Complex with OX40L

MM25PL3A2 Fab (VH and VL domains comprising the sequences of SEQ ID Nos: 97 and 96, respectively) was complexed with OX40L in 1.2:1 stoichiometric molar ratio, incubated on ice for one hour. The complex was purified by size exclusion chromatography and analyzed on SDS PAGE. Purified protein was concentrated to 3 mg/ml in PBS. CryoEM grids (UltraAUFoil R 0.6/1 on 300 gold mesh) were screened at different concentrations (3 mg/ml, 1.0 mg/ml and 0.5 mg/ml). CryoEM data was collected on a grid frozen with a concentration of 1 mg/ml with a dose of 55 e-/Å² and a pixel size of 0.85 Å on Krios FEI microscope equipped with K3 detector. 3400 movies were collected at a tilt angle of zero degrees and 1200 images were collected at a tilt axis of 30 degrees to overcome the preferred orientation issue. All the data processing steps (motion correction, CTF estimation, particle picking, extraction, 2D classification, ab initio structure solution and 3D refinement of map) were performed in CryoSPARC. Initial map from 3400 micrographs displayed preferred orientation and therefore a tilted data set was collected to improve the resolution. The resolution of the final map according to 0.143 gold standard cutoff was calculated to be 3.94 Å (FIG. 4). Alphafold2 generated Fab model and OX40L coordinates (PDB ID: 2HEV) were fintted into the cryoEM map using the programs, ChimeraX and Coot followed by real space refinement in Phenix.refine. The refined model was analyzed in MoE after energy minimization to map the epitope and paratope residues. The definition for a residue to be part of the epitope/paratope was a distance of <=4.6 Å to the respective other molecule.

### Results

### Generation of MM25PL3A2

The OX40L binder generation workflow described in the methods section above led to the discovery of the binder MM25PL3A2 (VH and VL domains comprising the sequences of SEQ ID Nos: 97 and 96, respectively). A further variant of this binder, MM25PL3A2_V1 (VH and VL domains comprising the sequences of SEQ ID Nos: 95 and 96, respectively) was prepared by introducing a N30S amino acid exchange in the VH.

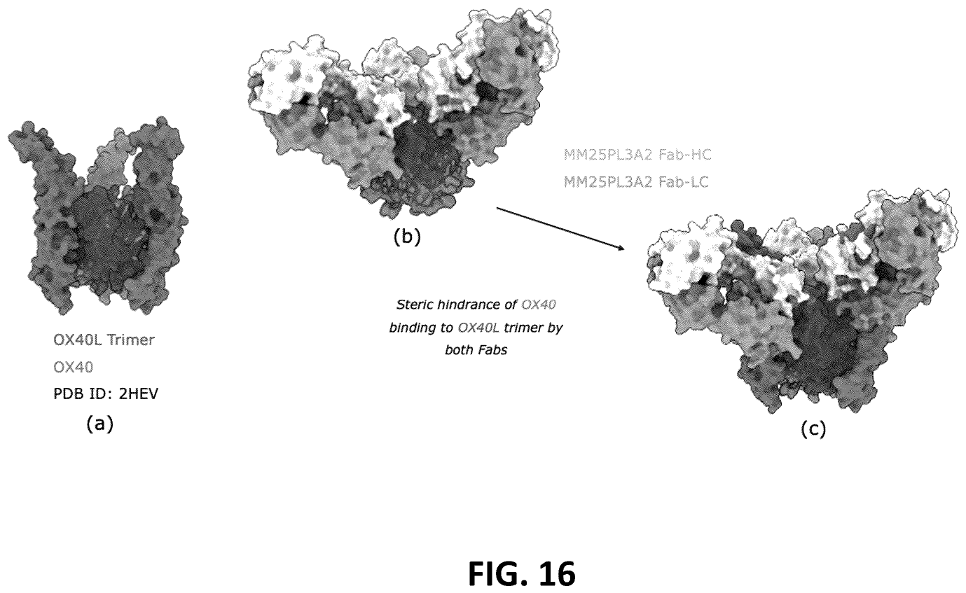

MM25PL3A2 and MM25PL3A2_V1 showed high binding affinity to OX40L (KD=1.72 E-10 M) and high efficacy in inhibiting OX40L-mediated signaling (see, e.g. Tables 9-11). Moreover, they were found to bind to an epitope on OX40L which is particularly suitable to prevent the interaction of OX40L and OX40 (FIGS. 16 & 17).

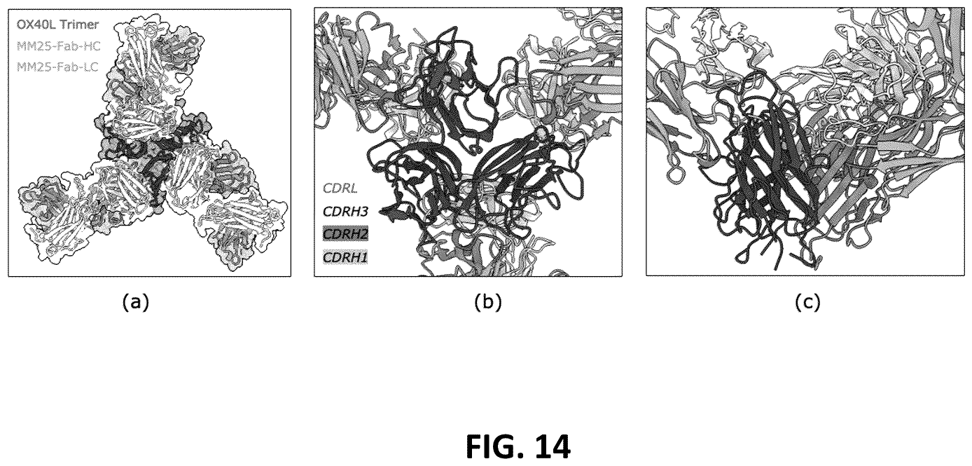

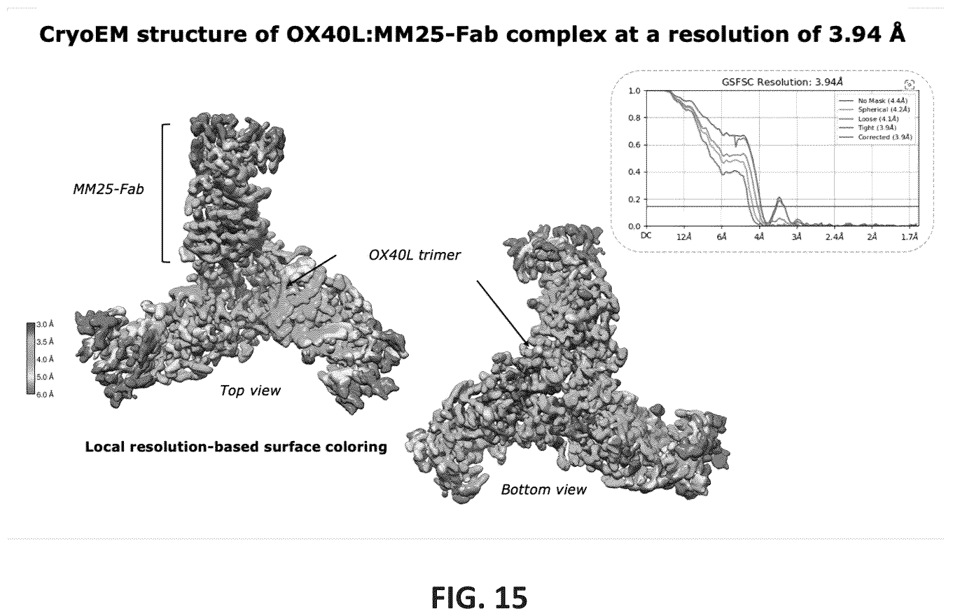

The CryoEM structure (at a resolution of 3.94 Å) of OX40L in complex with MM25PL3A2 Fab was found to display a 3:3 binding stoichiometry (FIG. 15). Top and side views indicated that the CDR loops intercalate between the OX40L monomers (FIG. 14). The epitope of the MM25PL3A2 Fab bound to OX40L was found to partially overlap with that of OX40 (FIG. 16). Therefore, the Fabs are thought to sterically hinder binding of OX40 to the OX40L trimer.

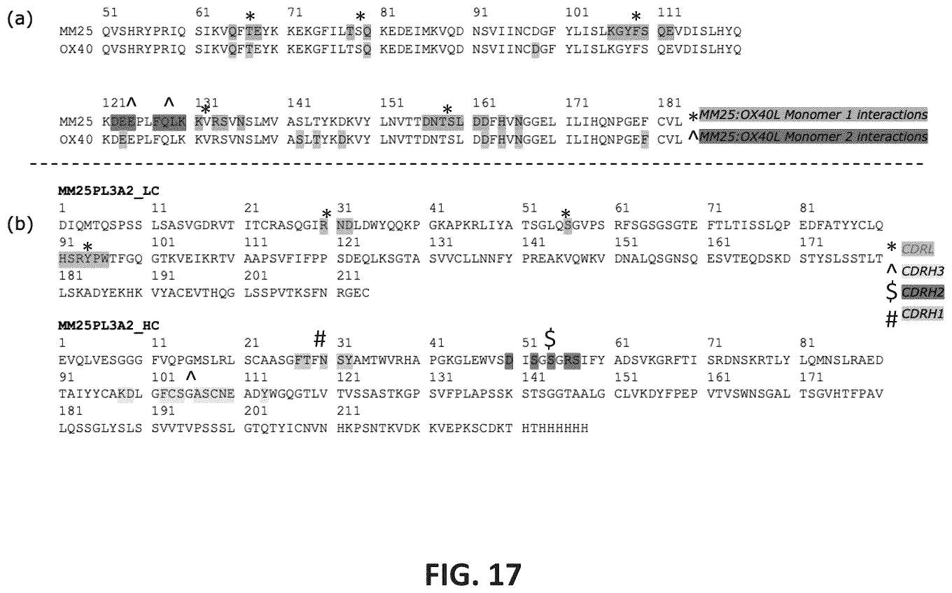

After energy minimization of CDR loops fitted into the 3.94 Å CryoEM map, and defining a residue as part of the epitope/paratope when within less than or equal to 4.6 Å to the other molecule, the MM25PL3A2 Fab epitope was mapped onto the human OX40L sequence, and the MM25PL3A2 Fab paratope residues were mapped onto the V_{H} and VL domain sequences (FIG. 17). These results indicated that the following residues of human OX40L are part of the MM25PL3A2 Fab epitope (numbering according to SEQ ID NO:110): Q65, T67, E68, T78, Q80, K106, G107, Y108, F109, S110, Q111, E112, D122, E123, E124, F127, Q128, L129, K130, K131, R133, 5134, N136, D156, N157, T158, 5159, L160, D161, D162, H164, and N166. It should be noted that D122, E123, E124, F127, Q128, L129, and K130 are located on a different OX40L monomer than the other residues. For comparative purposes, FIG. 17 (at a) also indicates also the OX40L residues which bind to OX40 (lower of the two OX40L sequences, labelled “OX40L”). Further, the following residues of the MM25PL3A2 Fab VH domain were found to make up the OX40L paratope: F27, T28, N30, 531, Y32, D50, S52, 554, R56, 557, K98, D99, F102, C103, 5104, A106, 5107, C108, N109, E110, and Y113 (consecutive numbering of SEQ ID NO:97 as shown in FIG. 17, positions can be easily renumbered according known antibody numbering schemes, such as Kabat or IMGT). The following residues of the MM25PL3A2 Fab V_{L} domain were found to make up the OX40L paratope: R30, N31, D32, S56, H91, S92, R93, Y94, P95, and W96 (consecutive numbering of SEQ ID NO:96 as shown in FIG. 17, positions can be easily renumbered according known antibody numbering schemes, such as Kabat or IMGT). Via modelling it was determined that the exchange of N30 for S did not alter the epitope. As N30, also S30 was determined to be part of the paratope.

Sequences from FIG. 17

(SEQ ID NO: 111)
QVSHRYPRIQSIKVQFTEYKKEKGFILTSQKEDEIMKVQDNSVIINCDG
FYLISLKGYFSQEVDISLHYQKDEEPLFQLKKVRSVNSLMVASLTYKDK
VYLNVTTDNTSLDDFHVNGGELILIHQNPGEFCVL
(SEQ ID NO: 112)
DIQMTQSPSSLSASVGDRVTITCRASQGIRNDLDWYQQKPGKAPKRLIY
ATSGLQSGVPSRFSGSGSGTEFTLTISSLQPEDFATYYCLQHSRYPWTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
(SEQ ID NO: 113)
EVOLVESGGGFVQPGMSLRLSCAASGFTFNSYAMTWVRHAPGKGLEWVS
DISGSGRSIFYADSVKGRFTISRDNSKRTLYLQMNSLRAEDTAIYYCAS
KDLGFCSGASCNEADYWGQGTLVTVSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKKVEPKSCDKTHTHHHHHH

### Example 4: Single Target Assays Examining In Vitro Pharmacology of Trispecific Antibodies

A trispecific antibody that targets IL-4 and IL-13 and a third antigen (TSLP) was measured for the binding affinity to its targets and efficacy for TARC inhibition in two in-vitro assays. The tested molecule corresponded to the molecular format shown in FIG. 4, with V_{H1} and V_{L1} being ADI-46653 D111E, V_{H2} and V_{L2} being CL-203143 V3.4, and the OX40L-binding Fab replaced by a TSLP-binding Fab.

### Materials and Methods

### In Vitro Human and Cyno IL-4, IL-13 and TSLP Binding Assay

Kinetic binding analysis was performed on a Biacore 8K (Cytiva) with HEPES-buffered saline with 3 mM EDTA and 0.05% tween-20 (HBS-EP+) as the running buffer, unless otherwise noted. For IL-4 and IL-13 binding, a CM5 Series S sensor chip (Cytiva) was immobilized with 50 ug/mL anti-human IgG (Fab specific) (Jackson Immunoresearch) in 10 mM sodium acetate pH 4.5 using amine coupling to a final density of 10,000-12,000 RU. For TSLP, protein A/G (Pierce 21186) was directly coupled under the same conditions to a surface density of 2,200-2,500 RU. Multi-cycle kinetics was performed with each antibody and a concentration series of human and cyno IL-4, IL-13 and TSLP antigens with regeneration after each antigen injection. The antibodies were captured at 0.5-3 ug/mL depending on format for 60 sec at a 10 μL/min flow rate to a density of 50-200 RU. A 1:3 serial dilution in HBS-EP+ of IL-4 starting at 5 nM, IL-13 starting at 33.3 nM or TSLP starting at 10 nM was prepared for a total of 8 concentrations, including a 0 nM injection for double referencing. Each injection of antigen was performed for an association time of 5 mins and a dissociation time of 40 and 2 mins for IL-4 and TSLP and 15 mins for IL-13 at a flow rate of 30 μL/min. After each antigen injection, the surface was regenerated with 2 injections of 10 mM glycine pH 1.5 (Cytiva) for 30 sec at 30 μL/min for subsequent antibody capture and antigen kinetics cycles. The data was analyzed in the Biacore Insight Evaluation software (Cytiva) using a 1:1 binding model.

### In Vitro Efficacy Assays

IL-4RI+Human PBMCs or IL-4RII+ human lung fibroblast cell line A549 were stimulated with 6 ng/mL of IL-4 or 10 ng/mL of IL-13 in the presence or absence of indicated antibody for 48 h. Supernatants were collected, and the amounts of secreted TARC or eotaxin-3 in the supernatant were measured using ELISA with chemiluminescence detection respectively (MesoScale Discovery).

### Results

To determine the single antigen binding of the IL-4xIL-13xTSLP CODV trispecific antibody, the binding analysis was performed on a Biacore 8K+. The resulting sensorgrams were double-referenced and fitted to a 1:1 binding model to determine kₐ, k_{d}, and KD. The TSLP TriAb can bind all three antigens at high affinity shown in table below for both human and Cyno species.

A summary of affinity values is shown in Table 16.

TABLE 16
Affinity values of IL-4 × IL-13 × TSLP trispecific antibody
for binding analysis for both human and cyno species.

| | | Capture | | | | | | |
|---|---|---|---|---|---|---|---|---|
| | | Level | Kₐ | K_{d} | K_{D} | | | |
| Construct | | (RU) | [1/M*s] | [1/s] | [M] | % Rmax | Rmax | Chi\hat{ }2 |
| human | IL-4 | 184 | 3.15E+06 | 1.00E−05* | 3.17E−12 | 109.7 | 18.8 | 0.059 |
| | IL-13 | 183 | 3.95E+05 | 1.52E−04 | 3.84E−10 | 97.0 | 13.5 | 0.175 |
| | TSLP | 189 | 2.46E+07 | 4.24E−04 | 1.72E−11 | 129.4 | 22.1 | 0.127 |
| Cyno | IL-4 | 186 | 1.81E+06 | 2.22E−05 | 1.23E−11 | 116.9 | 20.1 | 0.394 |
| | IL-13 | 184 | 4.02E+05 | 2.99E−04 | 7.44E−10 | 84.5 | 11.8 | 0.117 |
| | TSLP | 384 | 5.30E+06 | 5.58E−04 | 1.05E−10 | 119.2 | 42.2 | 0.315 |

*Below the limit of detection of the Biacore 8K+ at the performed dissociation time.

The potency of the anti-IL-4/IL-13 or the anti-TSLP arm of the TSLP CODV triAb to block thymus and activation-regulated chemokine (TARC, also known as CCL17) and eotaxin-3 (also known as CCL26) secretion in response to IL-4 or IL-13 stimulation, or TSLP/TSLPR-stimulated TARC secretion were assessed using human PBMCs and A549 cellular assays. IC50 values listed in the table below were determined by testing TSLP CODV triAb in a 10-point serial dilution of dose starting at 100 nM to inhibit all three arms.

A summary of IC50 values is shown in Table 17.

TABLE 17
IC50 values of IL-4 × IL-13 × TSLP trispecific antibody
for binding analysis for both human and cyno species.

| | IL-4 IC₅₀ | IL-13 IC₅₀ | TSLP IC₅₀ |
|---|---|---|---|
| Construct | (ng/mL) | (ng/mL) | (ng/mL) |
| huPBMC | 0.07 | 0.11 | 2.47 |
| A549 | 0.7 | 1.40 | N/A |

### Example 5: Generation and Characterization of Trispecific Binding Proteins that Bind IL-4, IL-13, and IL-17

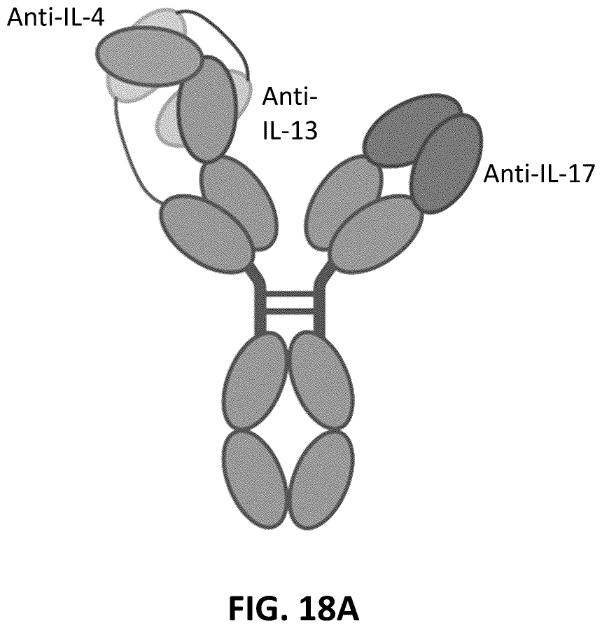

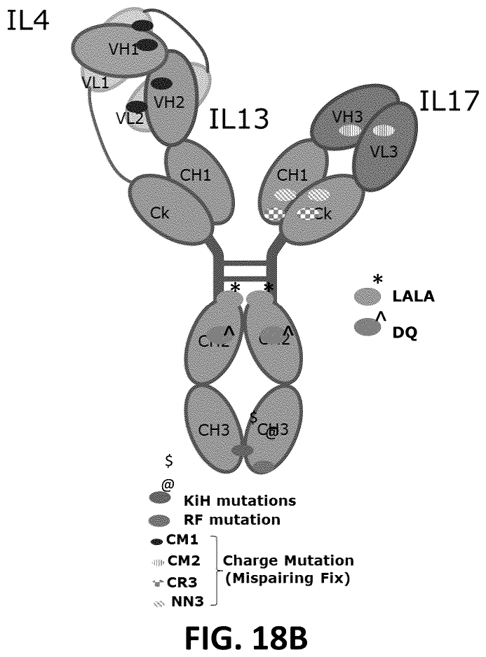

Trispecific binding proteins comprising a cross-over dual variable domain (CODV) arm and a traditional Fab arm were generated and tested for antigen-specific binding. A schematic of the binding protein design is shown in FIG. 18A, with anti-IL4/anti-IL13 CODV arm as described in Example 1 (see FIG. 1).

Mutations were introduced into the Fc domain to: promote association of the two heavy chains (S354C, T366W/Y349C, T366S, L368A, and Y407V; knobs-into-holes or KiH mutations); promote purification of the assembled binding protein (H435R/Y436F; RF mutation); to reduce effector function (L234A/L235A; LALA); and to extend half-life (T256D/T307Q; DQ), as shown in FIG. 18B.

Mutations were also introduced into the CODV arm ((39K (VH) and 38E (VL)); CM1), as well as into the Fab arm (39E (VH) and 38K (VL); CM2), (T192E (CH1), N137K (Ck) and S114A (Ck); CR3), and (K221E (CH1), K228D (CH1), E123K (Ck) and D122K (Ck); NN3), to promote proper chain pairing.

For the anti-IL-17 antigen binding domain, clones were generated from mice with human immunoglobulin locus sequences (see, e.g., U.S. Pat. No. 10,881,084) using a single B-cell antibody discovery approach. Three clones were generated: MMB05PL10_D10_v01-CM2 (SEQ ID Nos:132 and 133 for VHand VL domains, respectively), C_{L}-495369_VH_N52F_V_{L03}-CM2 (SEQ ID Nos:134 and 135 for VHand VL domains, respectively), and CL-495369_VH-wt-v1.1_VL-v3-CM2 (SEQ ID Nos:136 and 137 for V_{H} and VL domains, respectively). MMB05PL10_D10_v01-CM2 included an N83M mutation in the VH domain to remove a potential deamidation hotspot. CL-495369_VH_N52F_V_{L03}-CM2 included an N52F substitution in CDR-H2 and multiple mutations in the VLframework to remove potential antigenicity risks. CL-495369 VH-wt-v1.1_VL-v3-CM2 included 5 mutations in the VHframework and 6 in the VLframework to remove potential antigenicity risks.

Trispecific binding proteins that bind IL-4, IL-13, and IL-17 were produced in the CODV format shown in FIG. 18A with the antigen binding domains summarized in Table 18. Production and purification of each binding protein from stable pools is shown in Table 19.

TABLE 18
Trispecific binding proteins tested.

| Binding | | | |
|---|---|---|---|
| Protein | IL4 | IL17 | IL13 |
| A1 | ADI- | MMB05PL10_D10_v01- | CL-203143_V3.4-CM1 |
| | 46659_D111S_CM1 | CM2 | (SEQ ID Nos: 72 and |
| | (SEQ ID Nos: 40 and | (SEQ ID Nos: 132 and | 73 for VH and VL, |
| | 41 for VH and VL, | 133 for VH and VL, | respectively) |
| | respectively) | respectively) | |
| B1 | | CL- | CL-203143_V3.4-CM1 |
| | | 495369_VH_N52F_VL03_{—} | (SEQ ID Nos: 72 and |
| | | CM2 | 73 for VH and VL, |
| | | (SEQ ID Nos: 134 and | respectively) |
| | | 135 for VH and VL, | |
| | | respectively) | |
| C1 | | CL-495369_VH-wt- | CL-203143_V3.4-CM1 |
| | | v1.1_VL-v3-CM2 | (SEQ ID Nos: 72 and |
| | | (SEQ ID Nos: 136 and | 73 for VH and VL, |
| | | 137 for VH and VL, | respectively) |
| | | respectively) | |
| D1 | ADI- | CL- | CL-203143_V3.4-CM1 |
| | 46653_D111E_CM1 | 495369_VH_N52F_VL03_{—} | (SEQ ID Nos: 72 and |
| | (SEQ ID Nos: 53 and | CM2 | 73 for VH and VL, |
| | 41 for VH and VL, | (SEQ ID Nos: 134 and | respectively) |
| | respectively) | 135 for VH and VL, | |
| | | respectively) | |

TABLE 19
Trispecific binding protein production and purification.

| | | | | SDS | | Sample | | | |
|---|---|---|---|---|---|---|---|---|---|
| | Sample | SEC | MS - | Page | | yield | SEC | | MS- |
| Binding | yields | % | Intact | (non- | aHIC | (% | % | aHIC | Intact |
| protein | (mg/L) | purity | % | Red %) | % | proA) | purity | % | % |
| A1 | 169 | 52.0 | 89.6* | — | 65 | 14% | 97.3 | 91.9 | 100% |
| B1 | 180 | 58.4 | 78** | 58.0 | 68 | 14% | 96.1 | 87.3 | 100% |
| C1 | 217 | 58.3 | 84** | 54.3 | 52 | 9% | 96.1 | 90.5 | 100% |
| D1 | 284 | 42 | 58.9*** | 40 | 50.2 | 9% | 95 | 93.1 | 100% |

MS mispairing identification
*(1X HC1, 1X HC2, 2X LC2)
**(1X HC1, 1XLC1; 1X HC1, 1XHC2, 2X LC2)
***(1X HC1, 1XLC1; free LC2)

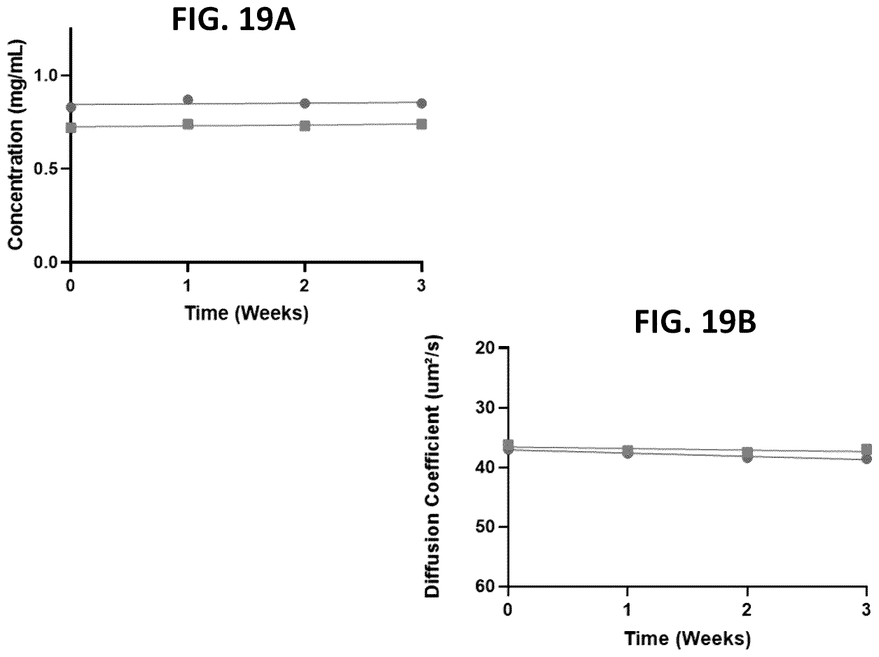

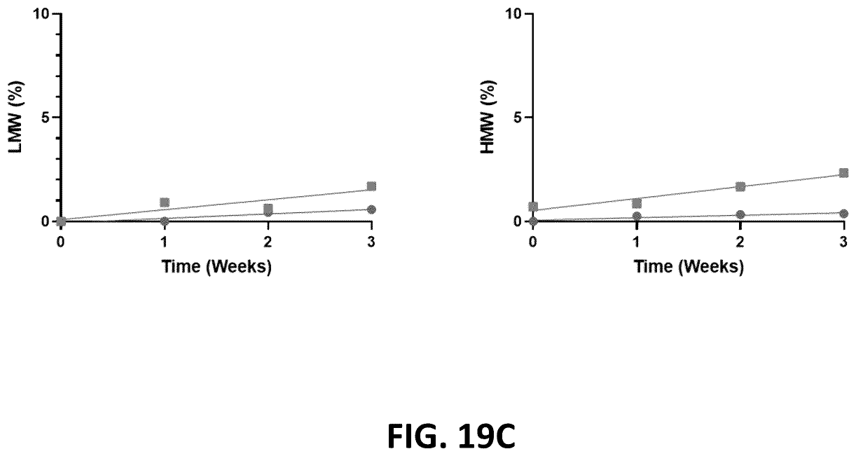

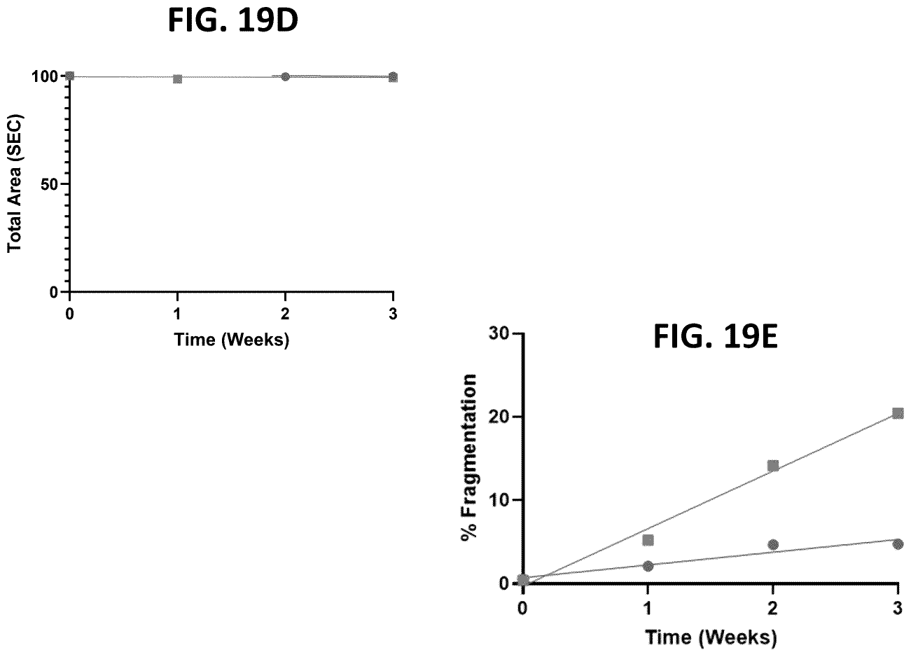

To summarize production, all 4 binding proteins showed≥95% main peak by analytical SEC, good physical stability, <30 mg/mL final yield, ~12-21% loss of active concentration (IL-4 & IL-13) at pH 5, ~18% loss of active concentration (IL-4 & IL-13) at pH 8, and high viscosity/turbidity at −150 mg/mL. Binding proteins A1, B1, and C1 showed D107 isomerization in the anti-IL-4 binding domain at pH 5, while binding proteins A1 and D1 showed the same at pH 8. Binding protein B1 showed ~9-18% loss of active concentration (IL-17AF) at pH 5. Binding proteins B1 and D1 showed ~12-15% loss of active concentration (IL-17AA) at pH 5 and D107 isomerization in the anti-IL-17 binding domain at pH 5. Stability of trispecific binding protein C1 over time as assessed by various parameters is shown in FIGS. 19A-19E.

Overall, binding protein A1 showed the lowest purity, while binding protein C1 had the lowest amount of fragments. All binding proteins showed approximately the same reduction sensitivity (above threshold of 0.7 mM DTT), increase in polydispersity index at low pH hold, freeze/thaw stability, and deviceability (viscosity). Binding proteins B1 and C1 showed highest stability upon agitation.

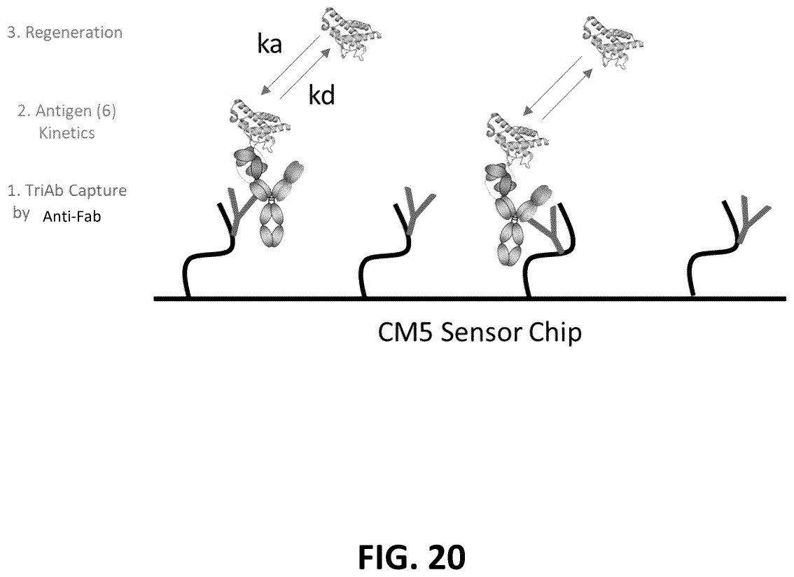

Antigen binding domains were tested in a trispecific CODV format (see FIG. 18A) for single antigen binding properties using surface plasmon resonance (SPR) as diagrammed in FIG. 20. Briefly, anti-human Fab antibodies were used to capture trispecific binding proteins. Trispecific binding proteins were used at varying concentrations and captured for 60 seconds at 10 μL/min. SPR was performed using a Biacore™ (Cytiva Life Sciences) 8K+ SPR system and CM5 sensor chip. Running buffer was HBS-EP+, pH 7.4. Immobilization conditions were 50 ug/mL streptavidin in acetate, pH 4.5 to ~2000-2500 RU with CaptureSelect™ (ThermoFisher Scientific) Human Fab-kappa biotin conjugates at 2 ug/mL in HBS-FP+ for 3 mins to 2000-2500 RU. Antigens were tested in HBS-FP+at 30L in the following ranges with 300 second association and 2400/120 second (IL-4) or 1200 second (all others) disassociation:
IL-4: 1:4 serial dilution from 5 nM to 19.7 pM
IL-13: 1:3 serial dilution from 33.3 nM to 45 pM
IL-17: 1:3 serial dilution from 33.3 nM to 45 pM

Regeneration was performed by 2 injections of 10 mM glycine pH 1.5 for 5 seconds at 30 μL/min. Capture concentrations were tailored to have a maximum signal of less than 20 RU. All experiments were performed in triplicate and averaged, and tested at 2 C or 37 C. Human and cynomolgus IL-4 were tested as monomers with a His-tag; human and cynomolgus IL-13 were tested as monomers with a His-tag; and human and cynomolgus IL-17 were tested as dimers with no tag. Monospecific, bispecific, and trispecific antibodies used for testing antigen binding are shown in Table 20.

TABLE 20
Molecules tested and target-specific capture concentrations.

| | | | | IL4 | IL13 | NKG2D | OX40L | IL17 |
|---|---|---|---|---|---|---|---|---|
| | | Molecular | | Capture | Capture | Capture | Capture | Capture |
| | Concentration | Weight | | Conc | Conc | Conc | Conc | Conc |
| Full Antibody Name | (mg/mL) | (Da) | Targets | (ug/mL) | (ug/mL) | (ug/mL) | (ug/mL) | (ug/mL) |
| Anti-IL4_hu8D4- | 0.224 | 145972 | IL4 | 2.5 | | | | |
| 8_IgG1_LALA_dK | | | | | | | | |
| a-IL4(hu8D4-8) × a-IL13(hzBB13)- | 0.100 | 197909 | IL4, | 2.5 | 2.5 | | | |
| CODV[10_10_0_0]_huIgG4_PE_dK | | | IL13 | | | | | |
| Anti-IL-13_comparator A_IgG1- | 0.520 | 49562 | IL13 | | 0.5 | | | |
| Fab::TEV-His | | | | | | | | |
| Anti-IL-13_comparator A_IgG1- | 0.820 | 145205 | IL13 | | 1.5 | | | |
| LALA-dK | | | | | | | | |
| Anti-IL-13_comparator B_IgG1- | 0.550 | 144061 | IL13 | | 2 | | | |
| LALA-dK | | | | | | | | |
| a-IL17A_comparator | 0.160 | 49507.23 | IL17 | | | | | 0.25 |
| A_huIgG1_Fab-His | | | | | | | | |
| a-IL17A_comparator A_huIgG1_dK | 0.160 | 146995 | IL17 | | | | | 0.75 |
| anti-IL17A-(comparator B)-huIgG1 | 0.150 | 147583.69 | IL17 | | | | | 1 |
| anti-IL17A-(comparator C)- | 0.080 | 146224 | IL17 | | | | | 0.5 |
| huIGHG4-P | | | | | | | | |
| (a-IL4_ADI-46659_D111S-CM1 × | 0.208 | 172354.67 | IL4, | 3 | 2.5 | | | 2.5 |
| a-IL13_CL-203143_V3.4-CM1)- | | | IL13, | | | | | |
| CODV × a-IL17A_CL-495369_VH- | | | IL17 | | | | | |
| wt-v1.1_VL-v3-CM2)-huIgG1- | | | | | | | | |
| CR3_NN3_LALA_DQ_dk | | | | | | | | |

Results for IL-13 are shown in Table 21. CL-203143_V3.4 showed slightly weaker affinity to IL-13 than the anti-IL-13 humanized monoclonal antibody comparator A (IgG) at 25° C., but tighter affinity at 37° C. CL-203143_V3.4 showed higher affinity binding than anti-IL-4/IL-13 bispecific comparator and IL-13 human monoclonal antibody comparator B, and had a cyno-to-human ratio of ~2 (weaker to cyno).

TABLE 21
Specific binding to IL-13 as measured by SPR (values refer to K_{D}).

| | Anti-IL-4/IL-13 | Anti-IL-13 | Anti-IL-13 | |
|---|---|---|---|---|
| | bispecific | comparator | comparator | CL-203143_{—} |
| | comparator | B | A | V3.4 |
| 25° C. | 1040 ± 248 pM | 194 ± 50 pM | 48 ± 20 pM | 88 ± 22 pM |
| 37° C. | 3300 ± 414 pM | 917 ± 166 pM | 287 ± 19 pM | 116 ± 2 pM |

Results for IL-4 are shown in Table 22. ADI-46659_D111S showed higher affinity than 8D4-8 regardless of temperature or species. Both ADI-46659_D111S and 8D4-8 had a cyno-to-human ratio of ~1 (equal affinity). ADI-46659_D111S showed possibly higher affinity at 37° C. as compared to 25° C., while 8D4-8 was the opposite.

TABLE 22
Specific binding to IL-4 as measured by SPR (values refer to K_{D}).

| | Anti-IL-4/IL-13 | | | |
|---|---|---|---|---|
| | bispecific | 8D4-8 | 8D4-8 | ADI- |
| | comparator | (IgG) | (TriAb) | 46659_D111S |
| 25° C. | 16 ± 4 pM | 39 ± 8 pM | 22 ± 11 pM | 9 ± 8 pM |
| 37° C. | 28 ± 3 pM | 92 ± 7 pM | 26 ± 1 pM | 3.5 ± 2.1 pM |

Results for IL-17 are shown in Table 23. At 25° C., CL-495369_VH-wt-v1.1_VL-v3 showed similar binding affinity as compared to anti-IL-17 comparator B but ~2-fold weaker affinity than anti-IL-17 comparator A or C. However, at 37° C., the affinity of CL-495369_VH-wt-v1.1_VL-v3 did not change, while anti-IL-17 comparator A showed higher affinity, and both anti-IL-17 comparators B and C showed significantly (>5-fold) weaker affinity.

TABLE 23
Specific binding to IL-17 as measured by SPR (values refer to K_{D}).

| | Anti-IL-17 | | Anti-IL-17 | | Anti-IL-17 | | CL-495369_{—} | |
|---|---|---|---|---|---|---|---|---|
| | comparator | | comparator | | comparator | | VH-wt-v1.1_{—} | |
| | A | | B | | C | | VL-v3 | |
| 25° | 9 ± 4 | pM | 16.8 ± 2.8 | pM | 8.3 ± 3.5 | pM | 20 ± 2 | pM |
| C. | | | | | | | | |
| 37° | 2.3 ± 1.0 | pM | 464 ± 21 | pM | 40 ± 5 | pM | 16.7 ± 0.1 | pM |
| C. | | | | | | | | |

In conclusion, replicates were similar for all antibodies and antigens, and % Rmax was close to or slightly above 100% for all antibodies to all targets. Tuning the capture concentration of all antibodies successfully kept the Rmax to below 20-25RU to minimize any avidity effects from dimeric antigens (e.g., IL-17) and non-specific contributions. The effects of increased temperature were mixed. In general, the newly generated sequences had similar or higher affinity as compared to the appropriate comparator.

The binding affinity of trispecific anti-IL-4/IL-13/IL-17 antibodies was also compared to monospecific and bispecific comparators. Binding of monospecific hu8D4-8 IgG1 to IL-4 showed a 1.8-fold reduction in affinity at 37° C. (Table 24). Binding of bispecific anti-IL-4 (hu8D4-8) x anti-IL-13 (hzBB13) CODV antibody showed 2.4-fold reduction in binding to IL-4 at 37° C. (Table 25) and 3.2-fold reduction in binding to IL-13 at 37° C. (Table 26). Binding of monospecific anti-IL-13 comparator A Fab to IL-13 showed a 2.3-fold enhancement in affinity at 37° C. (Table 27), whereas binding of monospecific anti-IL-13 comparator A IgG1 to IL-13 showed a 5.9-fold reduction in affinity at 37° C. (Table 28). Binding of monospecific anti-IL-13 comparator B IgG1 to IL-13 showed a 4.7-fold reduction in affinity at 37° C. (Table 29). Binding of monospecific anti-IL-17 comparator A Fab to IL-17 showed a 1.4-fold enhancement in affinity at 37° C. (Table 30), while binding of monospecific anti-IL-17 comparator A IgG1 to IL-17 showed a 4.0-fold enhancement in affinity at 37° C. (Table 31). Binding of monospecific anti-IL-17 comparator B IgG1 to IL-17 showed a 2.8-fold reduction in affinity at 37° C. (Table 32). Binding of monospecific anti-IL-17 comparator C IgG4 to IL-17 showed a 4.9-fold reduction in affinity at 37° C. (Table 33).

Binding of trispecific CODV binding protein (a-IL4_ADI-46659_D111S-CM1 x a-IL13_CL-203143_V3.4-CM1)-CODV x a-IL17A_CL-495369_VH-wt-v1.1_VL-v3-CM2)-huIgGl-CR3_NN3_LALA_DQ_dk showed 2.7-fold enhancement in binding affinity to IL-4 at 37° C. (Table 34), 1.9-fold reduction in binding affinity to IL-13 at 37° C. (Table 35), and 1.2-fold enhancement in binding affinity to IL-17 at 37° C. (Table 36). This binding protein was configured according to FIG. 18A with 4 polypeptide chains: a CODV light chain comprising SEQ ID NO:142, a CODV heavy chain comprising SEQ ID NO:143, a Fab heavy chain comprising SEQ ID NO:144, and a Fab light chain comprising SEQ ID NO:145.

TABLE 24
Binding of anti-IL-4 hu8D4-8 IgG1 monospecific antibody to human/cyno IL-4 as measured by SPR.

| | | | Capture | | | | | | | | | | | | |
|---|---|---|---|---|---|---|---|---|---|---|---|---|---|---|---|
| | | | Level | | | | | | Affinity | | Rmax | | | | |
| Temp | | | (RU) ± | | On Rate | | Off Rate | | (pM) ± | | (RU) ± | | % Rmax ± | | |
| (° C.) | Antigen | Chi2 | SD | | (1/Ms) ± SD | | (1/s) ± SD | | SD | | SD | | SD | | Comment |
| 25 | CYNO | 5.61E−01 | 127.2 | 6.7 | 1.68E+07 | 6.23E+06 | 3.31E−04 | 1.44E−04 | 19.4 | 1.5 | 17.6 | 1.4 | 126.4 | 13.8 | Binder, |
| | | | | | | | | | | | | | | | Good Fit |
| | HUMAN | 4.97E−01 | 129.3 | 10.4 | 2.84E+07 | 2.75E+06 | 4.64E−04 | 1.45E−04 | 16.1 | 3.8 | 17.9 | 3.1 | 127.4 | 33.2 | Binder, |
| | | | | | | | | | | | | | | | Good Fit |
| 37 | HUMAN | 7.95E−01 | 128.7 | 12.3 | 2.47E+07 | 9.24E+06 | 6.78E−04 | 1.97E−04 | 28.3 | 3.4 | 23.4 | 2.2 | 82.5 | 0.2 | Binder, |
| | | | | | | | | | | | | | | | Good Fit |

TABLE 25
Binding of anti-IL-4 (hu8D4-8) × anti-IL-13 (hzBB13) CODV bispecific antibody to human/cyno IL-4 as measured by SPR.

| | | | Capture | | | | | | | | | | | | |
|---|---|---|---|---|---|---|---|---|---|---|---|---|---|---|---|
| | | | Level | | | | | | Affinity | | Rmax | | | | |
| Temp | | | (RU) ± | | On Rate | | Off Rate | | (pM) ± | | (RU) ± | | % Rmax ± | | |
| (° C.) | Antigen | Chi2 | SD | | (1/Ms) ± SD | | (1/s) ± SD | | SD | | SD | | SD | | Comment |
| 25 | CYNO | 6.82E−02 | 68.5 | 1.4 | 8.17E+06 | 9.48E+05 | 3.27E−04 | 7.44E−05 | 41.1 | 13.3 | 9.6 | 0.9 | 174.3 | 19.4 | Binder, |
| | | | | | | | | | | | | | | | Good Fit |
| | HUMAN | 6.94E−02 | 68.4 | 1.6 | 1.14E+07 | 4.82E+06 | 4.67E−04 | 2.75E−04 | 39.2 | 8.4 | 9.9 | 1.1 | 178.2 | 24.1 | Binder, |
| | | | | | | | | | | | | | | | Good Fit |
| 37 | HUMAN | 1.72E−02 | 67.0 | 5.3 | 7.57E+06 | 3.22E+05 | 6.99E−04 | 8.06E−05 | 92.2 | 6.6 | 11.5 | 0.8 | 105.9 | 0.8 | Binder, |
| | | | | | | | | | | | | | | | Good Fit |

TABLE 26
Binding of anti-IL-4 (hu8D4-8) × anti-IL-13 (hzBB13) CODV bispecific antibody to human/cyno IL-13 as measured by SPR.

| | | | Capture | | | | | | | | | | | | |
|---|---|---|---|---|---|---|---|---|---|---|---|---|---|---|---|
| | | | Level | | | | | | Affinity | | Rmax | | | | |
| Temp | | | (RU) ± | | On Rate | | Off Rate | | (pM) ± | | (RU) ± | | % Rmax ± | | |
| (° C.) | Antigen | Chi2 | SD | | (1/Ms) ± SD | | (1/s) ± SD | | SD | | SD | | SD | | Comment |
| 25 | CYNO | 3.09E−02 | 54.7 | 9.1 | 8.56E+05 | 9.04E+04 | 1.09E−03 | 9.27E−05 | 1290 | 197 | 9.5 | 1.7 | 130.3 | 2.6 | Binder, |
| | | | | | | | | | | | | | | | Good Fit |
| | HUMAN | 7.80E−02 | 73.6 | 15.6 | 3.78E+05 | 5.05E+04 | 3.85E−04 | 4.43E−05 | 1040 | 248 | 15.0 | 3.4 | 152.5 | 8.1 | Binder, |
| | | | | | | | | | | | | | | | Good Fit |
| 37 | HUMAN | 4.07E−02 | 59.5 | 8.2 | 4.46E+05 | 1.36E+04 | 1.48E−03 | 2.03E−04 | 3300 | 414 | 10.7 | 1.7 | 134.8 | 3.8 | Binder, |
| | | | | | | | | | | | | | | | Good Fit |

TABLE 27
Binding of anti-IL-13 comparator A Fab to human/cyno IL-13 as measured by SPR.

| | | | Capture | | | | | | | | | | | | |
|---|---|---|---|---|---|---|---|---|---|---|---|---|---|---|---|
| | | | Level | | | | | | Affinity | | Rmax | | | | |
| Temp | | | (RU) ± | | On Rate | | Off Rate | | (pM) ± | | (RU) ± | | % Rmax ± | | |
| (° C.) | Antigen | Chi2 | SD | | (1/Ms) ± SD | | (1/s) ± SD | | SD | | SD | | SD | | Comment |
| 25 | CYNO | 2.06E−02 | 28.1 | 0.5 | 6.21E+05 | 2.16E+04 | 2.79E−05 | 8.69E−06 | 45.2 | 15.8 | 9.5 | 0.3 | 127.4 | 1.1 | Binder, |
| | | | | | | | | | | | | | | | Good Fit |
| | HUMAN | 5.99E−02 | 26.9 | 0.5 | 3.42E+05 | 3.16E+04 | 3.38E−05 | 1.35E−05 | 98.6 | 38.4 | 11.3 | 0.1 | 157.4 | 2.5 | Binder, |
| | | | | | | | | | | | | | | | Good Fit |
| 37 | HUMAN | 3.31E−02 | 34.6 | 1.1 | 4.03E+05 | 1.71E+04 | 1.68E−05 | 5.20E−06 | 42.0 | 14.5 | 13.4 | 0.5 | 145.5 | 1.2 | Binder, |
| | | | | | | | | | | | | | | | Good Fit |

TABLE 28
Binding of anti-IL-13 comparator A IgG1 to human/cyno IL-13 as measured by SPR.

| | | | Capture | | | | | | | | | | | | |
|---|---|---|---|---|---|---|---|---|---|---|---|---|---|---|---|
| | | | Level | | | | | | Affinity | | Rmax | | | | |
| Temp | | | (RU) ± | | On Rate | | Off Rate | | (pM) ± | | (RU) ± | | % Rmax ± | | |
| (° C.) | Antigen | Chi2 | SD | | (1/Ms) ± SD | | (1/s) ± SD | | SD | | SD | | SD | | Comment |
| 25 | CYNO | 3.94E−02 | 62.6 | 3.2 | 7.07E+05 | 4.89E+04 | 1.20E−05 | 3.10E−06 | 17.0 | 4.7 | 14.3 | 0.6 | 126.4 | 0.8 | Binder, |
| | | | | | | | | | | | | | | | Good Fit |
| | HUMAN | 4.02E−02 | 63.8 | 5.0 | 3.32E+05 | 3.99E+04 | 1.65E−05 | 8.54E−06 | 48.4 | 20.1 | 17.1 | 0.8 | 148.1 | 4.7 | Binder, |
| | | | | | | | | | | | | | | | Good Fit |
| 37 | HUMAN | 8.62E−02 | 170.7 | 8.2 | 4.29E+05 | 1.32E+04 | 1.23E−04 | 1.12E−05 | 287 | 18.5 | 23.5 | 1.2 | 76.0 | 0.5 | Binder, |
| | | | | | | | | | | | | | | | Good Fit |

TABLE 29
Binding of anti-IL-13 comparator B IgG1 to human/cyno IL-13 as measured by SPR.

| | | | Capture | | | | | | | | | | | | |
|---|---|---|---|---|---|---|---|---|---|---|---|---|---|---|---|
| | | | Level | | | | | | Affinity | | Rmax | | | | |
| Temp | | | (RU) ± | | On Rate | | Off Rate | | (pM) ± | | (RU) ± | | % Rmax ± | | |
| (° C.) | Antigen | Chi2 | SD | | (1/Ms) ± SD | | (1/s) ± SD | | SD | | SD | | SD | | Comment |
| 25 | CYNO | 5.79E−02 | 45.8 | 1.5 | 3.38E+06 | 4.32E+05 | 1.85E−03 | 1.36E−04 | 552 | 64 | 7.7 | 0.2 | 92.7 | 4.1 | Binder, |
| | | | | | | | | | | | | | | | Good Fit |
| | HUMAN | 1.31E−01 | 44.5 | 1.0 | 1.86E+06 | 1.34E+05 | 3.60E−04 | 9.41E−05 | 194 | 50 | 9.4 | 0.5 | 115.3 | 3.4 | Binder, |
| | | | | | | | | | | | | | | | Good Fit |
| 37 | HUMAN | 1.28E−01 | 79.7 | 7.4 | 2.71E+06 | 1.02E+06 | 2.38E−03 | 4.55E−04 | 917 | 166 | 15.2 | 2.1 | 104.0 | 4.7 | Binder, |
| | | | | | | | | | | | | | | | Good Fit |

TABLE 30
Binding of anti-IL-17 comparator A Fab to human/cyno IL-17 as measured by SPR.

| | | | Capture | | | | | | | | | | | | |
|---|---|---|---|---|---|---|---|---|---|---|---|---|---|---|---|
| | | | Level | | | | | | Affinity | | Rmax | | | | |
| Temp | | | (RU) ± | | On Rate | | Off Rate | | (pM) ± | | (RU) ± | | % Rmax ± | | |
| (° C.) | Antigen | Chi2 | SD | | (1/Ms) ± SD | | (1/s) ± SD | | SD | | SD | | SD | | Comment |
| 25 | CYNO | 4.79E−02 | 42.7 | 1.8 | 1.36E+06 | 5.82E+04 | 1.07E−05 | 5.33E−06 | 7.9 | 4.0 | 22.7 | 1.6 | 87.1 | 2.8 | Binder, |
| | | | | | | | | | | | | | | | Good Fit |
| | HUMAN | 1.49E−01 | 43.5 | 0.1 | 1.65E+06 | 5.67E+04 | 1.41E−05 | 7.71E−06 | 8.5 | 4.4 | 23.0 | 0.5 | 86.4 | 1.8 | Binder, |
| | | | | | | | | | | | | | | | Good Fit |
| 37 | HUMAN | 8.35E−02 | 57.1 | 3.8 | 2.67E+06 | 1.11E+05 | 1.56E−05 | 3.33E−06 | 5.9 | 1.5 | 30.1 | 2.1 | 86.2 | 0.7 | |

TABLE 31
Binding of anti-IL-17 comparator A IgG1 to human/cyno IL-17 as measured by SPR.

| | | | Capture | | | | | | | | | | | | |
|---|---|---|---|---|---|---|---|---|---|---|---|---|---|---|---|
| | | | Level | | | | | | Affinity | | Rmax | | | | |
| Temp | | | (RU) ± | | On Rate | | Off Rate | | (pM) ± | | (RU) ± | | % Rmax ± | | |
| (° C.) | Antigen | Chi2 | SD | | (1/Ms) ± SD | | (1/s) ± SD | | SD | | SD | | SD | | Comment |
| 25 | CYNO | 1.25E−01 | 93.5 | 0.6 | 1.38E+06 | 9.37E+03 | 9.79E−06 | 1.84E−06 | 7.1 | 1.4 | 36.2 | 0.3 | 94.3 | 0.7 | Binder, |
| | | | | | | | | | | | | | | | Good Fit |
| | HUMAN | 2.05E−01 | 94.3 | 1.7 | 1.83E+06 | 4.61E+03 | 1.67E−05 | 7.54E−06 | 9.1 | 4.1 | 35.0 | 0.5 | 90.3 | 0.7 | Binder, |
| | | | | | | | | | | | | | | | Good Fit |
| 37 | HUMAN | 1.54E−01 | 116.1 | 3.6 | 2.66E+06 | 9.41E+03 | 6.10E−06 | 2.72E−06 | 2.3 | 1.0 | 43.2 | 1.3 | 90.4 | 0.2 | |

TABLE 32
Binding of anti-IL-17 comparator B IgG1 to human/cyno IL-17 as measured by SPR.

| | | | Capture | | | | | | | | | | | | |
|---|---|---|---|---|---|---|---|---|---|---|---|---|---|---|---|
| | | | Level | | | | | | Affinity | | Rmax | | | | |
| Temp | | | (RU) ± | | On Rate | | Off Rate | | (pM) ± | | (RU) ± | | % Rmax ± | | |
| (° C.) | Antigen | Chi2 | SD | | (1/Ms) ± SD | | (1/s) ± SD | | SD | | SD | | SD | | Comment |
| 25 | CYNO | 4.94E−03 | 75.0 | 0.2 | 1.69E+05 | 7.77E+03 | 3.95E−03 | 1.96E−04 | 23300 | 107 | 23.6 | 0.7 | 77.0 | 2.3 | Binder, |
| | | | | | | | | | | | | | | | Good Fit |
| | HUMAN | 4.92E−02 | 75.3 | 0.8 | 2.33E+05 | 3.17E+03 | 3.92E−05 | 6.09E−06 | 168 | 28 | 27.5 | 0.2 | 89.0 | 1.5 | Binder, |
| | | | | | | | | | | | | | | | Good Fit |
| 37 | HUMAN | 1.43E−01 | 95.5 | 2.6 | 4.54E+05 | 3.76E+03 | 2.11E−04 | 7.96E−06 | 464 | 21 | 35.8 | 0.9 | 91.4 | 0.3 | |

TABLE 33
Binding of anti-IL-17 comparator C IgG4 to human/cyno IL-17 as measured by SPR.

| | | | Capture | | | | | | | | | | | | |
|---|---|---|---|---|---|---|---|---|---|---|---|---|---|---|---|
| | | | Level | | | | | | Affinity | | Rmax | | | | |
| Temp | | | (RU) ± | | On Rate | | Off Rate | | (pM) ± | | (RU) ± | | % Rmax ± | | |
| (° C.) | Antigen | Chi2 | SD | | (1/Ms) ± SD | | (1/s) ± SD | | SD | | SD | | SD | | Comment |
| 25 | CYNO | 8.41E−02 | 70.5 | 4.6 | 1.24E+06 | 7.28E+04 | 4.05E−05 | 4.06E−06 | 32.7 | 2.5 | 26.7 | 2.2 | 91.7 | 1.5 | Binder, |
| | | | | | | | | | | | | | | | Good Fit |
| | HUMAN | 4.94E−01 | 72.5 | 4.8 | 2.08E+06 | 2.35E+05 | 1.67E−05 | 6.03E−06 | 8.3 | 3.5 | 27.6 | 2.4 | 91.8 | 2.0 | Binder, |
| | | | | | | | | | | | | | | | Good Fit |
| 37 | HUMAN | 1.19E−01 | 93.6 | 8.5 | 3.52E+06 | 1.43E+05 | 1.41E−04 | 1.43E−05 | 40.3 | 5.2 | 32.6 | 2.4 | 84.3 | 1.8 | |

TABLE 34
Binding of trispecific CODV binding protein (a-IL4_ADI-46659_D111S-CM1 ×
a-IL13_CL-203143_V3.4-CM1)-CODV × a-IL17A_CL-495369_VH-wt-v1.1_VL-v3-CM2)-
huIgG1-CR3_NN3_LALA_DQ_dk to human/cyno IL-4 as measured by SPR.

| | | | Capture | | | | | | | | | | | | |
|---|---|---|---|---|---|---|---|---|---|---|---|---|---|---|---|
| | | | Level | | | | | | Affinity | | Rmax | | | | |
| Temp | | | (RU) ± | | On Rate | | Off Rate | | (pM) ± | | (RU) ± | | % Rmax ± | | |
| (° C.) | Antigen | Chi2 | SD | | (Ms) ± SD | | (1/s) ± SD | | SD | | SD | | SD | | Comment |
| 25 | CYNO | 6.88E−02 | 153.7 | 1.3 | 1.51E+06 | 4.36E+05 | 2.51E−05 | 2.16E−05 | 19.7 | 17.4 | 11.4 | 1.9 | 79.9 | 13.7 | Binder, |
| | | | | | | | | | | | | | | | Good Fit |
| | HUMAN | 1.11E−01 | 153.3 | 1.5 | 3.14E+06 | 8.08E+05 | 2.51E−05 | 2.19E−05 | 9.3 | 8.2 | 12.4 | 3.0 | 86.7 | 21.8 | Binder, |
| | | | | | | | | | | | | | | | Good Fit |
| 37 | HUMAN | 6.21E−02 | 191.0 | 10.7 | 4.22E+06 | 2.62E+05 | 1.44E−05 | 8.75E−06 | 3.5 | 2.1 | 20.3 | 1.3 | 114.2 | 1.1 | |

TABLE 35
Binding of trispecific CODV binding protein (a-IL4_ADI-46659_D111S-CM1 ×
a-IL13_CL-203143_V3.4-CM1)-CODV × a-IL17A_CL-495369_VH-wt-v1.1_VL-v3-CM2)-
huIgG1-CR3_NN3_LALA_DQ_dk to human/cyno IL-13 as measured by SPR.

| | | | Capture | | | | | | | | | | | | |
|---|---|---|---|---|---|---|---|---|---|---|---|---|---|---|---|
| | | | Level | | | | | | Affinity | | Rmax | | | | |
| Temp | | | (RU) ± | | On Rate | | Off Rate | | (pM) ± | | (RU) ± | | % Rmax ± | | |
| (° C.) | Antigen | Chi2 | SD | | (Ms) ± SD | | (1/s) ± SD | | SD | | SD | | SD | | Comment |
| 25 | CYNO | 1.12E−02 | 108.2 | 1.5 | 1.50E+06 | 5.16E+03 | 2.05E−04 | 8.09E−06 | 136 | 5.8 | 9.5 | 0.1 | 115.4 | 0.4 | Binder, |
| | | | | | | | | | | | | | | | Good Fit |
| | HUMAN | 3.98E−02 | 108.2 | 1.6 | 1.19E+06 | 3.11E+03 | 7.38E−05 | 5.35E−06 | 61.8 | 4.6 | 10.0 | 0.1 | 121.5 | 0.6 | Binder, |
| | | | | | | | | | | | | | | | Good Fit |
| 37 | HUMAN | 4.04E−02 | 177.3 | 5.2 | 1.07E+06 | 2.28E+04 | 1.23E−04 | 3.09E−06 | 115 | 4.7 | 14.9 | 0.6 | 109.7 | 1.1 | |

TABLE 36
Binding of trispecific CODV binding protein (a-IL4_ADI-46659_D111S-CM1 ×
a-IL13_CL-203143_V3.4-CM1)-CODV × a-IL17A_CL-495369_VH-wt-v1.1_VL-v3-CM2)-
huIgG1-CR3_NN3_LALA_DQ_dk to human/cyno IL-17 as measured by SPR.

| | | | Capture | | | | | | | | | | | | |
|---|---|---|---|---|---|---|---|---|---|---|---|---|---|---|---|
| | | | Level | | | | | | Affinity | | Rmax | | | | |
| Temp | | | (RU) ± | | On Rate | | Off Rate | | (pM) ± | | (RU) ± | | % Rmax ± | | |
| (° C.) | Antigen | Chi2 | SD | | (Ms) ± SD | | (1/s) ± SD | | SD | | SD | | SD | | Comment |
| 25 | CYNO | 5.77E−02 | 135.4 | 2.6 | 2.41E+06 | 5.31E+04 | 6.80E−05 | 6.90E−06 | 28.3 | 3.4 | 22.6 | 0.1 | 95.3 | 1.9 | Binder, |
| | | | | | | | | | | | | | | | Good Fit |
| | HUMAN | 7.48E−02 | 138.2 | 6.2 | 2.63E+06 | 7.65E+04 | 5.25E−05 | 5.25E−06 | 20.0 | 1.8 | 22.3 | 0.4 | 91.9 | 2.4 | Binder, |
| | | | | | | | | | | | | | | | Good Fit |
| 37 | HUMAN | 5.04E−02 | 177.8 | 4.8 | 3.98E+06 | 1.56E+05 | 6.65E−05 | 2.73E−06 | 16.7 | 0.1 | 26.6 | 0.6 | 85.3 | 0.4 | |

### Example 6: In Vitro and In Vivo Pharmacology of Trispecific Binding Proteins that Bind IL-4, IL-13, and IL-17

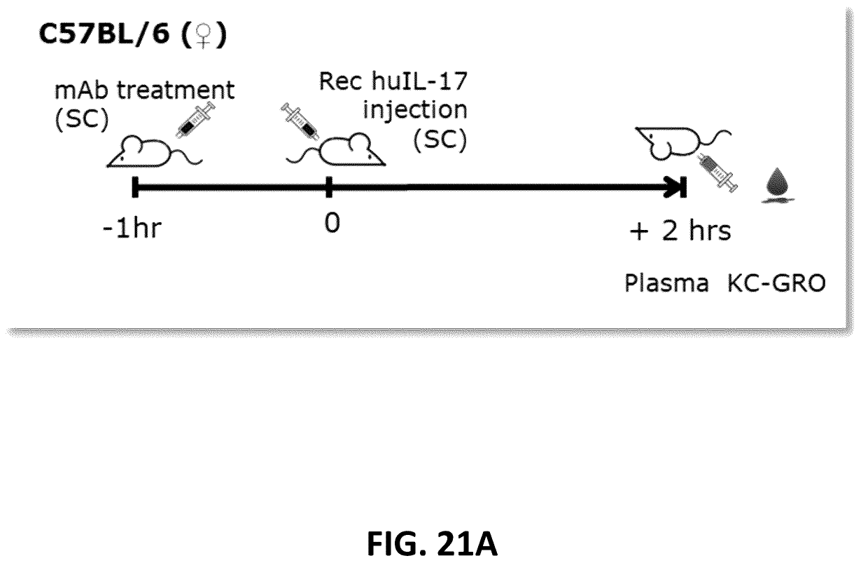

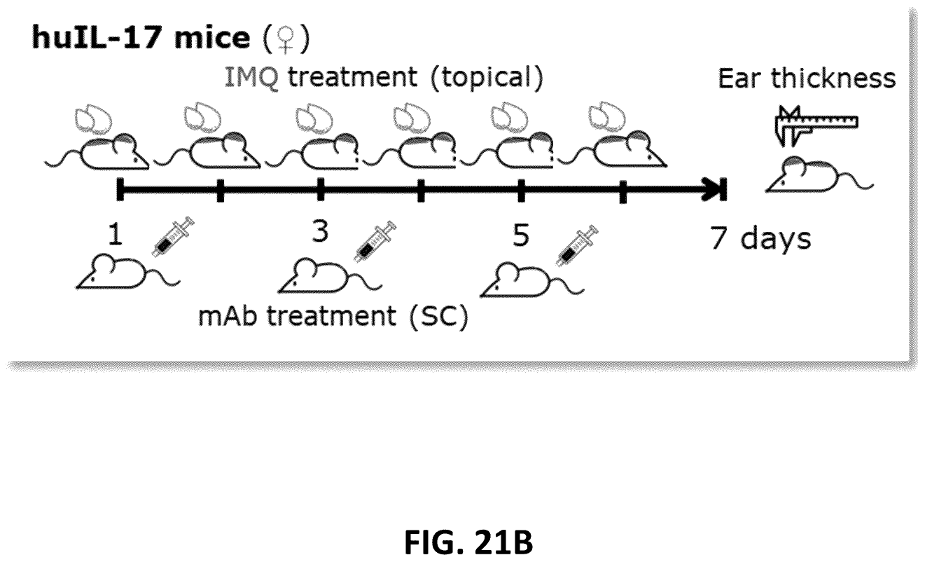

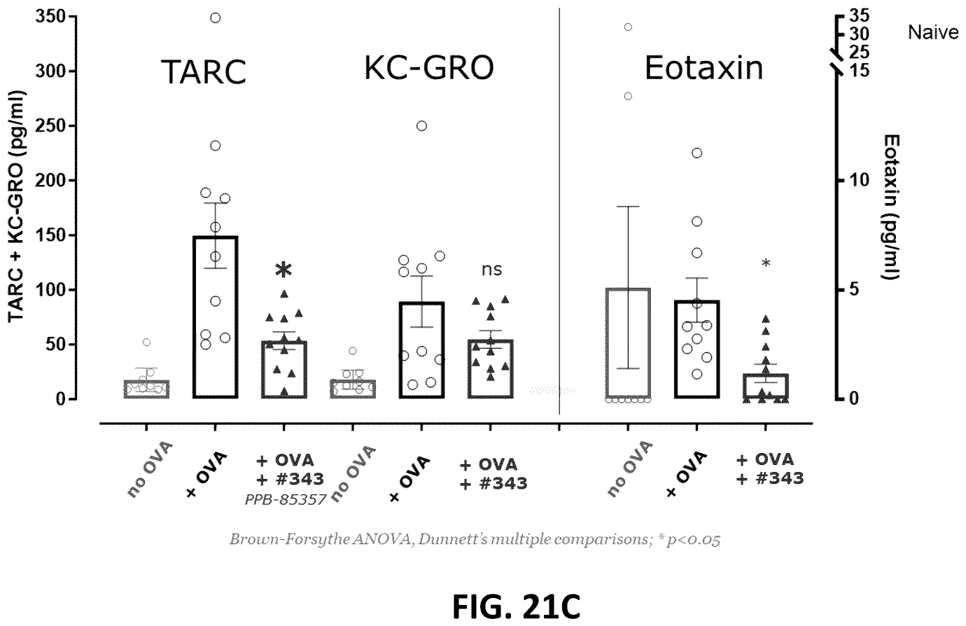

Next, effect of trispecific binding proteins on IL-17 activity was tested in vivo using two mouse models. The first assayed keratinocyte chemoattractant (KC)/growth-regulated oncogene (GRO) 2 hours after dosing mice with recombinant human IL-17 and 3 hours after dosing mice with anti-IL-4/IL-13/IL-17 trispecific binding protein A1, B1, C1, or D1 (FIG. 21A). The second assayed ear thickness (by AUC) from baseline to day 7 after daily topical treatment with imiquimod (IMQ) and three injections of anti-IL-4/IL-13/IL-17 trispecific binding protein A1, B1, C1, or D1 in a model of psoriatic skin inflammation (FIG. 21B). The results are shown in Table 37. These results demonstrate that the trispecific binding proteins were equivalent to benchmark in in vivo inhibition of IL-17.

TABLE 37
Results of in vivo IL-17 assays.

| | | | | | | | | In vivo inhibition 7-day IMQ | | | |
|---|---|---|---|---|---|---|---|---|---|---|---|
| | | | | In vivo inhibition single dose | | | | model | | | |
| | | | | IL17-induced KC/GRO @ 3 h | | | | AUC of ear thickness change | | | |
| | | | | post dose | | | | from BL to d 7 | | | |
| | | | | | | % of | | | | % of | |
| | | | | % INH | | comparator | | % INH | | comparator | |
| | | | | abs @ 10 | | B @ 10 | | abs @ 20 | | B @ 20 | |
| | Binder | | | mg/kg | 95% | mg/kg | 95% | mg/kg | 95% | mg/kg | 95% |
| | IL-13 | IL-4 | IL-17 | SC | CI | SC | CI | SC EOD | CI | SC EOD | CI |
| BP | CL- | ADI- | MMB05PL10_{—} | 83.2% | 24.7% | 95.1% | 28.3% | −8.1% | 17.2% | −16.7% | 35.5% |
| A1 | 203143_{—} | 46659_{—} | D10_v01- | | | | | | | | |
| | V3.4-CM1 | D111S- | CM2 | | | | | | | | |
| BP | | CM1 | CL- | 88.8% | 11.74% | 101.5% | 13.4% | 51.9% | 10.8% | 106.8% | 22.3% |
| B1 | | | 495369_VH_{—} | | | | | | | | |
| | | | N52F_VL03- | | | | | | | | |
| | | | CM2 | | | | | | | | |
| BP | | | CL- | 92.7% | 9.24% | 106% | 10.6% | 50.16% | 15.1% | 103.3% | 31% |
| C1 | | | 495369_VH- | | | | | | | | |
| | | | wt-v1.1_VL- | | | | | | | | |
| | | | v3-CM2 | | | | | | | | |
| BP | | ADI- | CL- | 69.4% | 15.6% | 79.4% | 17.8% | tbd | | tbd | |
| D1 | | 46653_{—} | 495369_VH_{—} | | | | | | | | |
| | | D111E- | N52F_VL03- | | | | | | | | |
| | | CM1 | CM2 | | | | | | | | |

| | | | | | | | | |
|---|---|---|---|---|---|---|---|---|
| IL-17 comparator B | 87.5% | 11.4% | 100% | 13.1% | 48.5% | 15.4% | 100% | 31.7% |

The effect of trispecific binding protein A1 on IL-4 and IL-13 signaling was also tested in humanized IL-4/IL-13/IL-4Ra mice. Mice were injected with OVA/alum, and levels of TARC, KC/GRO, and eotaxin were measured in BAL fluid. As shown in FIG. 21C, the trispecific binding protein was active in vivo in reducing TARC, KC/GRO, and eotaxin.

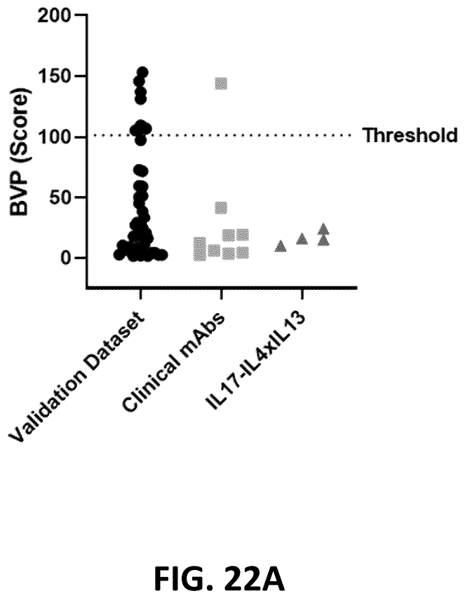

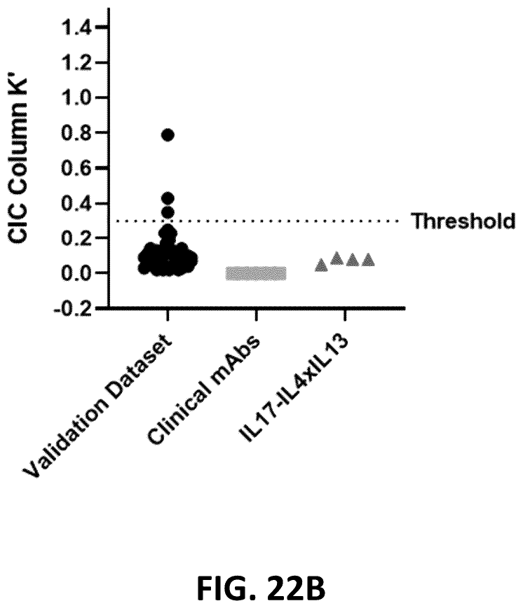

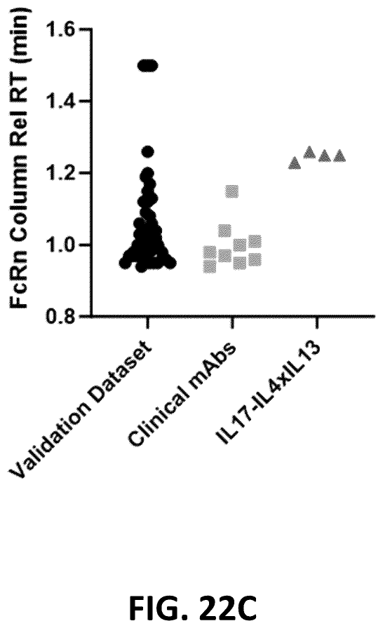

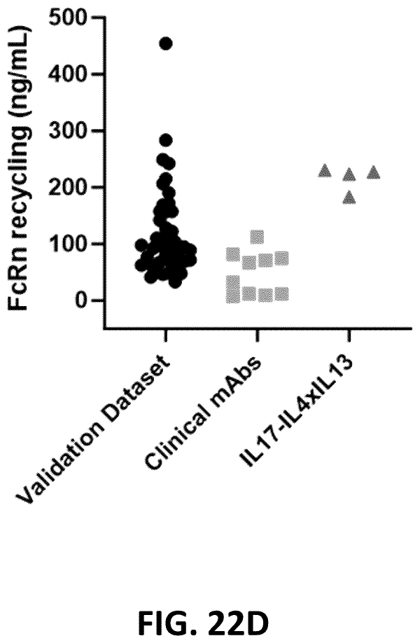

Additionally, a series of 4 in vitro assays was undertaken to assess physicochemical properties of trispecific binding proteins A1, B1, C1, and D1, e.g., to predict potential for PK liability risk. These included assays for non-specific binding (FIG. 22A), self-association (FIG. 22B), FcRn binding (FIG. 22C), and FcRn cellular recycling (FIG. 22D). The results (Table 38) showed that all 4 molecules demonstrated values below threshold for non-specific binding and self-association, indicating low risk for PK liability. Results from FcRn binding and cellular recycling were in line with historical data to date obtained for reference monoclonal antibodies.

TABLE 38
In vitro physicochemical property assays.

| Binding | BVP | | FcRn | FcRn |
|---|---|---|---|---|
| protein | Score | CIC k′ | Column | Cellular |
| A1 | 15 | 0.05 | 1.23 | 183.52 |
| B1 | 24 | 0.09 | 1.25 | 227.69 |
| C1 | 10 | 0.08 | 1.25 | 230.78 |
| D1 | 16 | 0.08 | 1.26 | 224.04 |
| Acceptance | <101.4 | <0.301 | <1.085** | <170.5** |
| Criteria* | | | | |

The four trispecific binding proteins were also tested for in vivo PK profile.

Each of trispecific binding proteins A1, B1, C1, or D1 was administered by Intravenous (IV) injection to three male Tg32 mice (see, e.g., Jackson Labs mouse strain number 014565) at 5 mg/kg dose. Serum was collected at 0.0833, 1, 4, 8, 24, 48, 72, 168, 216, 336, 504 and 672 hrs post-dose. The selected peptides (see Table 39) were measured by LC-MS method (Immune-capture/Bottom-up Approach) with LLOQ of 2500 ng/ml or lower (250 ng/ml) depending on peptide.

TABLE 39
LC-MS analysis of selected peptides.

| Binding | LC-MS method |
|---|---|
| protein | signature peptide |
| B1 | GLEWIGEIFHIGGTIYNPSLK.+3y10 (SEQ ID NO: 262) |
| | ITCSGDALPK.+2y8 (SEQ ID NO: 263) |
| | NTLYLQMNSLR.+2y8 (SEQ ID NO: 264) |
| | SGTASVVC[CAM]LLK.+2y7 (SEQ ID NO: 265) |
| | 2.TTPPVLDSDGSFFLVSK.+2y15+2 (SEQ ID NO: 266) |
| | 3.TTPPVLDSDGSFFLYSK.+2y15+2 (SEQ ID NO: 267) |
| | TVAAPAVFIFPPSK.+2y10 (SEQ ID NO: 268) |
| | VTISVDTSK.+2y7 (SEQ ID NO: 269) |
| | VYACEVTHQGLSSPVTK.+3y15+2 (SEQ ID NO: 270) |
| C1 | GLEWIGEINHIGGTIYNPSLK.+3y16+2 (SEQ ID NO: |
| | 271) |
| | GPSVFPLAPSSK.+2y7 (SEQ ID NO: 272) |
| | NDLGWYQK.+2y5 (SEQ ID NO: 273) |
| | NTLYLQMNSLR.+2y8 (SEQ ID NO: 264) |
| | SGTASVVC[CAM]LLK.+2y7 (SEQ ID NO: 265) |
| | SYELTQPPSVSVSPGQTAR.+2y13 (SEQ ID NO: 274) |
| | 2.TTPPVLDSDGSFFLVSK.+2y15+2 (SEQ ID NO: 266) |
| | 3.TTPPVLDSDGSFFLYSK.+2y15+2 (SEQ ID NO: 267) |
| | TVAAPAVFIFPPSK.+2y10 (SEQ ID NO: 268) |
| | VTISVDTSK.+2y7 (SEQ ID NO: 269) |
| | VYACEVTHQGLSSPVTK.+3y15 (SEQ ID NO: 270) |
| A1 | DSERPSGIPER.+2y10+2 (SEQ ID NO: 275) |
| | LLIYDASNLETGVPSR.+2y13 (SEQ ID NO: 276) |
| | SGTASVVC[CAM]LLNNFYPR.+2y10 (SEQ ID NO: 277) |
| | SYELTQPPSVSVSPGQTAR.+2y13 (SEQ ID NO: 274) |
| | TVAAPSVFIFPPSDEQLK.+2y8 (SEQ ID NO: 278) |
| | VYAC[CAM]EVTHQGLSSPVTK.+2y11 (SEQ ID NO: 279) |
| | LSSVTAADTAVYYC[CAM]AR.+2y12 (SEQ ID NO: 280) |
| | 2.TTPPVLDSDGSFFLYSK.+2y15+2 (SEQ ID NO: 267) |
| | 3.TTPPVLDSDGSFFLVSK.+2y15+2 (SEQ ID NO: 266) |
| | IEFPYTFGQGTK.+2y9 (SEQ ID NO: 281) |
| | KPGQSPQLLIYTFFYR.+2y11 (SEQ ID NO: 282) |
| | TVAAPAVFIFPPSK. +2y10 (SEQ ID NO: 268) |
| D1 | DSERPSGIPER.+2y10+2 (SEQ ID NO: 275) |
| | LLIYDASNLETGVPSR.+2y13 (SEQ ID NO: 276) |
| | SGTASVVC[CAM]LLNNFYPR.+2y10 (SEQ ID NO: 277) |
| | SYELTQPPSVSVSPGQTAR.+2y13 (SEQ ID NO: 274) |
| | TVAAPSVFIFPPSDEQLK.+2y8 (SEQ ID NO: 278) |
| | LSSVTAADTAVYYC[CAM]AR. +2y12 (SEQ ID NO: 280) |
| | TTPPVLDSDGSFFLYSK.+2y 15+2 (SEQ ID NO: 267) |
| | GFYPSDIAVEWESNGQPENNYK.+2y6 (SEQ ID NO: 283) |
| | 3.TTPPVLDSDGSFFLVSK.+2y15+2 (SEQ ID NO: 266) |
| | FLIFGTYSLQSGVPSR.+2y13 (SEQ ID NO: 284) |
| | SGTASVVC[CAM]LLK.+2y7 (SEQ ID NO: 265) |
| | TVAAPAVFIFPPSK. +2y10 (SEQ ID NO: 268) |

PK analysis was performed using Non compartmental analysis (Model 200-202 for plasma; linear up log down of Phoenix WinNonLin (version 8.2); nominal times were used).

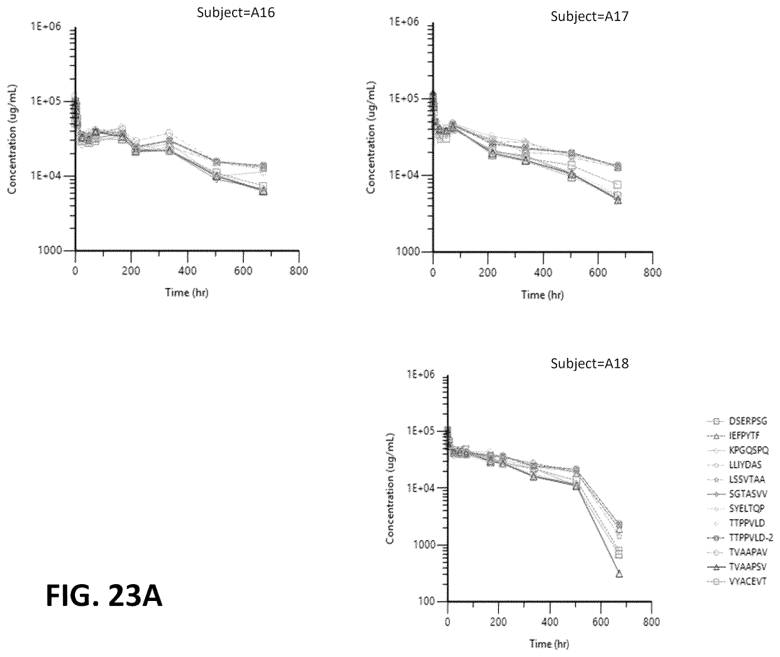

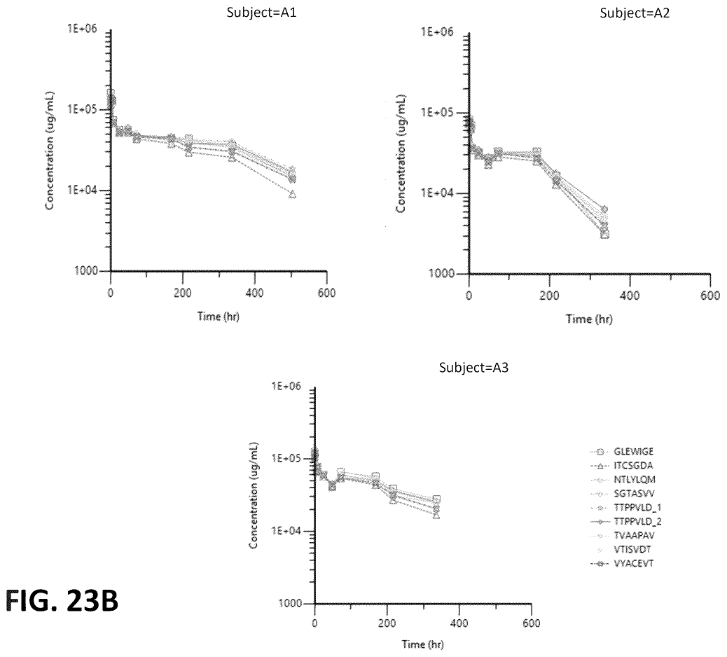

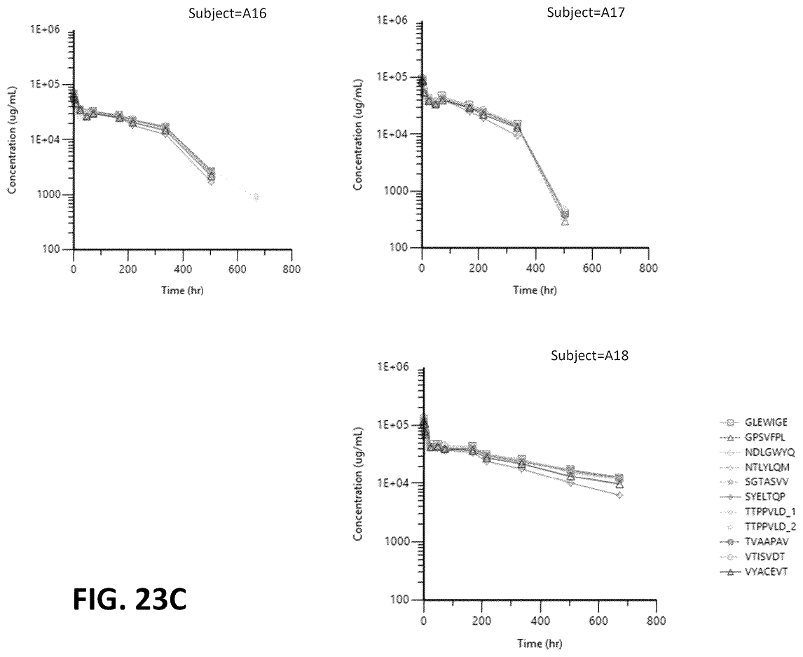

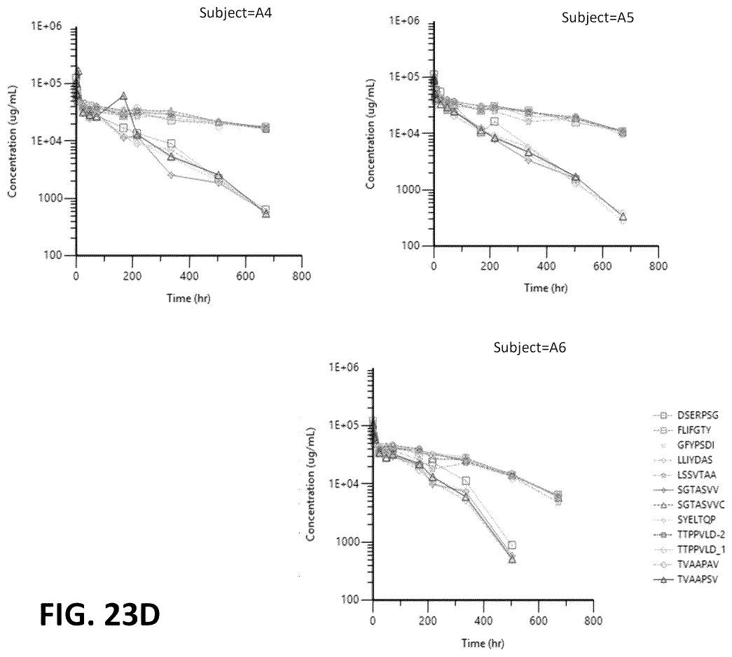

For binding protein B1, the overlap of concentration-time curve of different peptide (FIG. 23B) indicated minor loss of integrity. Final PK parameters were calculated from ITCSGDALPK (SEQ ID NO:263) signature peptide in the CODV IL13xIL4 light chain range, the average of animal 1 and 3 (animal 2 had potential ADA), was summarized as mean Co of 108 ug/mL, with mean AUCINF of 17900 hr*ug/ml, mean clearance of 0.281 mL/hr/kg, mean volume distribution of 70.9 mL/kg and mean half-life of 174 hr. In conclusion, binding protein B1 showed minor loss of integrity, mean clearance of 0.281 ml/h/kg, a mean volume distribution of 70.9 mL/kg and a mean half-life of 174 hr.

For binding protein C1, the overlap of concentration-time curve of different peptides (FIG. 23C) indicated minor loss of integrity. Final PK parameters were calculated from SYELTQPPSVSVSPGQTAR (SEQ ID NO:274) signature peptide in the CODV IL13xIL4 light chain range, animal 18 (animal 16 and animal 17 had potential ADA), was summarized as Co of 126 ug/mL, with a AUCINF of 16700 hr*ug/ml, a clearance of 0.300 mL/hr/kg, a volume distribution of 95.2 mL/kg. In conclusion, binding protein C1 showed minor loss of integrity, had a clearance of 0.300 ml/h/kg, a volume distribution of 95.2 mL/kg and a half-life of 229 hr.

For binding protein A1, the overlap of concentration-time curve of different peptides (FIG. 23A) indicated minor loss of integrity. Final PK parameters were calculated on the average of SYELTQPPSVSVSPGQTAR (SEQ ID NO:274) signature peptide from three animals (Animal #18 at 672 h was excluded due to potential ADA), summarized as mean Co of 102 ug/mL, with a mean AUCINF of 16400 hr*ug/ml, a mean clearance of 0.305 mL/hr/kg, a mean volume distribution of 103 mL/kg and a mean half-life of 228 hr. In conclusion, binding protein A1 showed minor loss of integrity, had mean clearance of 0.305 ml/h/kg, mean volume distribution of 103 mL/kg and mean half-life of 228 hr.

For binding protein D1, the overlap of concentration-time curve of different peptides (FIG. 23D), indicated major loss of integrity. Final PK parameters were calculated on the average of SYELTQPPSVSVSPGQTAR (SEQ ID NO:274) signature peptide from three animals, summarized as mean Co of 100 ug/mL, with mean AUCINF of 6820 hr*ug/ml, mean clearance of 0.742 mL/hr/kg, mean volume distribution of 108 mL/kg and mean half-life of 97.5 hr. In conclusion, binding protein D1 showed major loss of integrity, and had mean clearance of 0.742 ml/h/kg, mean volume distribution of 108 mL/kg and mean half-life of 97.5 hr.

### Example 7: Developability Assessment of Trispecific Binding Proteins that Bind IL-4, IL-13, and IL-17

Trispecific binding proteins A1, B1, C1, and D1 were subjected to further assays to profile their developability, e.g., expression characteristics, stress response, molecular profiling, stability, and deviceability. Descriptions of the assays used are provided in Example 1.

The trispecific binding proteins were profiled by capillary isoelectric focusing (cIEF), capillary zone electrophoresis (CZE), and hydrophobic interaction chromatography (HIC). These data are summarized in Table 40. The isoelectric point of binding proteins A1 and D1 was below 8. All binding proteins showed low charge, potentially predicting unfavorable colloidal properties.

TABLE 40
Molecular profiling of trispecific binding
proteins A1, B1, C1, and D1.

| | cIEF | CZE | HIC |
|---|---|---|---|
| Binding protein | pI | charge | Retention time (min) |
| A1 | 6.93 | 3.4 | 15.6 |
| B1 | 8.03 | 5.6 | 16.1 |
| C1 | 8.03 | 4.1 | 15.9 |
| D1 | 7.10 | 3.1 | 16.0 |

The trispecific binding proteins were also examined for deviceability characteristics, and the results are shown in Table 41.

TABLE 41
Deviceability assays for trispecific
binding proteins A1, B1, C1, and D1.

| Binding | Concentration | kD-DLS | Viscosity | μ-nephelometry |
|---|---|---|---|---|
| protein | (mg/mL) | (mL/g) | (cP) | (NTU) |
| A1 | 147 | −22.39 | 25.35 | 12.6 |
| B1 | 153 | −37.50 | 64.45 | 9.5 |
| C1 | 145 | −7.11 | 28.62 | 8.9 |
| D1 | 150 | −0.14 | 55.52 | 11.1 |

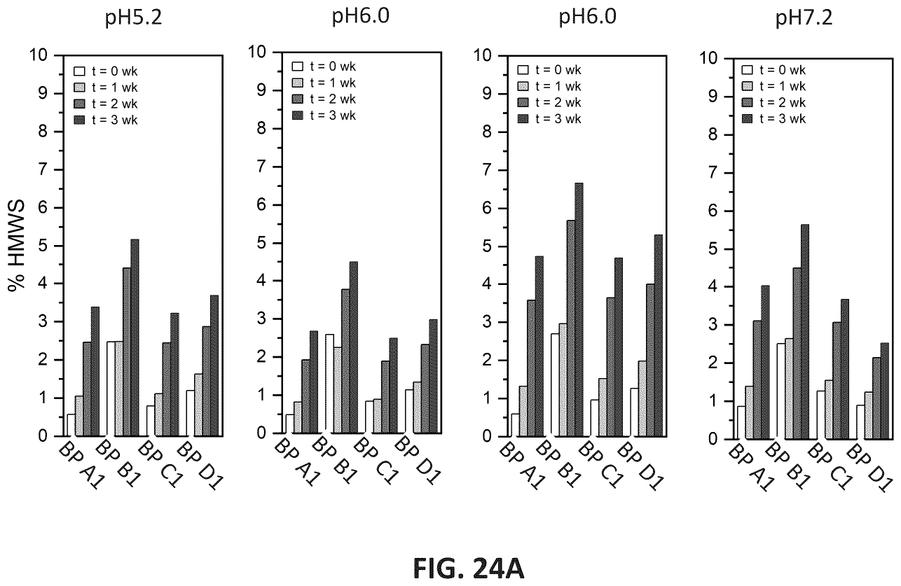

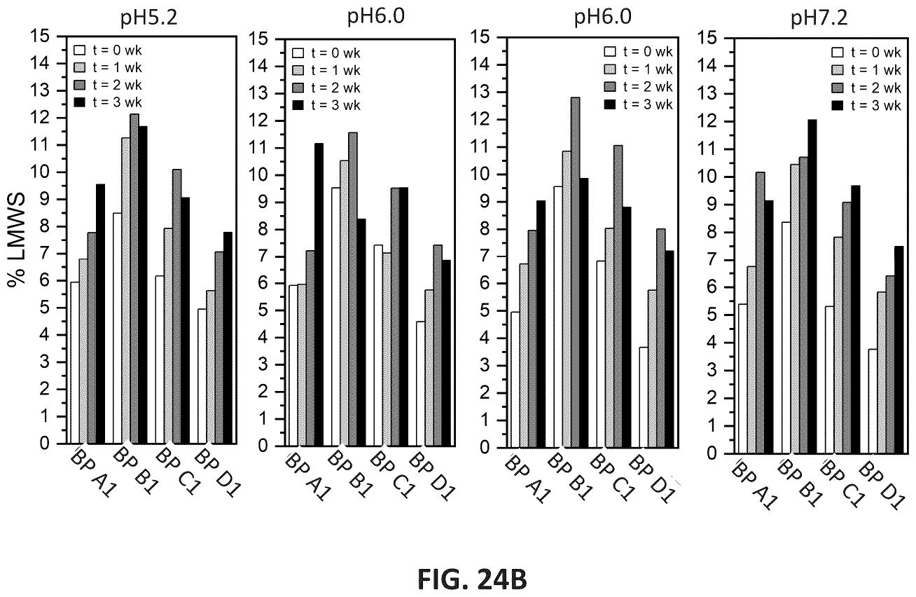

The trispecific binding proteins were also analyzed by SEC examining aggregation (FIG. 24A) and fragmentation (FIG. 24B) at pH values ranging from 5.2-7.2 for 3 weeks at 40° C. The results showed trending increases in aggregation and fragmentation for all binding proteins (>0.50%/week).

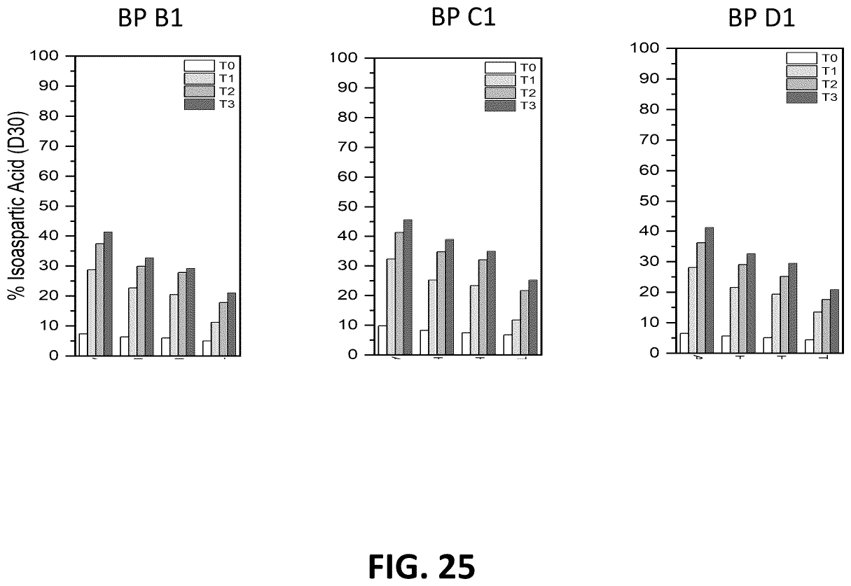

The chemical degradation of trispecific binding proteins was also analyzed, using isomerization of an aspartic acid residue in HCDR1 (D30) of the anti-IL-17 binding domain. As shown in FIG. 25, binding proteins B1, C1, and D1 showed elevated risk of isoaspartic acid formation at low pH.

In total, these results provide a deviceability profile for each binding protein. Considering expression level, sensitivity to DTT reduction, polydispersity index at low pH, agitation, freeze/thawing, stability, and deviceability, binding protein C1 had the most favorable characteristics.

### Example 8: Crystal Structures of IL-17A in Complex with Newly Generated Anti-IL-17 Antigen Binding Domains

### Materials and Methods

Co-structure of anti-IL17 FabB04 in complex with Human IL-17A (SEQ ID NO: 156 below) was prepared prior to crystallization, using a sitting drop vapor diffusion method. An X-ray data set was collected at SOLEIL PX-1 beamline at a wavelength of 0.978565 Å, using an Eiger-X 16M (Dectris Ltd.) detector. The X-ray data was processed and analyzed with the autoPROC toolbox, which relies on XDS and Aimless, and the phase problem was solved by molecular replacement using the Phaser program of the CCP4 suite, and PDB models of IL-17A and FabB04 from the Protein Data Bank (PDB code 4HR9 and 5GZ0, respectively). The co-complex model was refined using the Refine program, and by iteratively building the model in COOT until the refinement converged. The model was validated using Molprobity prior to analyzing the epitope/paratope interactions.

FabB04 comprises the following CDRs: a CDR-H1 comprising the amino acid sequence of SEQ ID NO:129, a CDR-H2 comprising the amino acid sequence of SEQ ID NO:130, a CDR-H3 comprising the amino acid sequence of SEQ ID NO:131, a CDR-L1 comprising the amino acid sequence of SEQ ID NO:126, a CDR-L2 comprising the amino acid sequence of GTY, and a CDR-L3 comprising the amino acid sequence of SEQ ID NO:128. Full amino acid sequences for the FabB04 heavy and light chains are provided below.

Heavy chain:
(SEQ ID NO: 154)
QVQLQQWGAGLLKPSETLSLTCAVFGGSFDGYYWNWIRQPPGKGLEWIGEINHIGGTIYNPS
LKSRVTISEDTSKNQFSLRLNSVTAADTATYYCARGITMGRGNYYYYGLDVWGQGTPVTVS
SASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTHHHHHH
Light chain:
(SEQ ID NO: 155)
DILMTQSPSSLSASVGDRVTITCRASQDIKNDLGWYQQKPGKVPKFLIFGTYSLQSGVPSRFSG
SGSGTDFTLTINSLQPEDFATYYCLQDNNYPRTFGQGTKVGFKRTVAAPSVFIFPPSDEQLKSG
TASVVCLLNNFYPREAKVQWKVDNALQSGNSQESVTEQDSKDSTYSLSSTLTLSKADYEKH
KVYACEVTHQGLSSPVTKSFNRGEC
hIL-17A
(SEQ ID NO: 156)
MGPNSEDKNF PRTVMVNLNI HNRNTNTNPK RSSDYYDRST SPWNLHRNED PERYPSVIWE
AKCRHLGCIN ADGNVDYHMN SVPIQQEILV LRREPPHSPN SFRLEKILVS VGCTCVTPIV
HHVA

### Results

The goal of these experiments was to characterize the binding mode, epitope, and paratope of the binding between anti-IL17 FabB04 and Human IL-17A. As shown in FIG. 26A, two Fabs bind to one IL-17A homodimer. Residues within 4A distance between human IL-17A chains A and B and FabB04 heavy and light chains are depicted in FIG. 26B.

FabB04 can interact with hIL-17A directly or via water molecules. Residues of the binding epitope (within 4A) in hIL-17A are shown in FIG. 26C for a first FabB04 molecule (FabB04, chains HL) and for a second FabB04 molecule (FabB04, chains IM) co-complexed with the two chains of the hIL-17A homodimer (chains A and B).

FIG. 26C, top sequence in upper panel (“B04-HL>IL17”), depicts the residues of the epitope in the hIL-17A homodimer (within 4A) to which the first FabB04 molecule (FabB04, chains HL) binds; residues of the epitope that are in hIL-17A chain A are depicted in different shaded text (namely L66, N70, D72, N74, D76, Y77, H78, M79, N80, V82, V116, T117, P118, I119, V120, H121, H122, V123, and A124; numbering based on SEQ ID NO:156), whereas residues of the epitope that are in hIL-17A chain B are depicted by highlighting (namely R31, Y35, R38, N70, A71, and D72; numbering based on SEQ ID NO:156).

FIG. 26C, bottom sequence in upper panel (“B04-IM>IL17”), depicts the residues of the epitope in the hIL-17A homodimer (within 4A) to which the second FabB04 molecule (FabB04, chains IM) binds; residues of the epitope that are in hIL-17A chain B are depicted in different shaded text (namely L66, N70, A71, D72, N74, D76, Y77, H78, M79, N80, V82, V116, T117, P118, 1119, V120, H121, H122, V123, and A124; numbering based on SEQ ID NO:156), whereas residues of the epitope that are in hIL-17A chain A are depicted by highlighting (namely N70, A71, and D72; numbering based on SEQ ID NO:156).

Residues of the binding paratope (within 4A) formed by the heavy and light chains of FabB04 are shown (by underlined text) in FIG. 26D.

Sequence comparison showed that key epitope residues are different between hIL-17A and hIL-17F. As such, without wishing to be bound to theory, it is thought that FabB04 is likely to bind more weakly to hIL-17AF and even more weakly to hIL-17F than to hIL-17A.

In summary, these experiments demonstrated that two Fabs bind to one hIL-17A homodimer. The binding epitope is composed of both chains of the dimer. The FabB04 heavy chain is the main component of the binding.

### Example 9: Generation and Characterization of Trispecific Binding Proteins that Bind IL-4, IL-13, and NKG2D

### Materials and Methods

### Analytical SEC

All size exclusion chromatography (SEC) analyses were done on an ACQUITY ultra-performance liquid chromatography system (UPLC; Waters Corp., Milford, MA) with a Superdex™ 200 Increase 5/150 GL SEC column (Sigma-Aldrich, Darmstadt, Germany) using phosphate buffered saline pH 7.2 as the mobile phase. For each sample, 10 μg of protein was injected in singlet over the column at 0.3 mL/min for 15 min and an absorbance at 280 nm (A280) was measured. Buffer blanks and Gel Filtration Standard (Bio-Rad, 1511901) were included at the beginning and end of the run. Resulting chromatograms were blank subtracted and manually integrated to determine percent purity.

For stressed samples, % HMW species were calculated as the sum of peaks with retention times less than the peak of interest. The % LMW species were calculated as the sum of peaks with retention times greater than the peak of interest. The total area was calculated by taking the area of the peak of interest of each time peak and dividing by the area of the TO peak of interest. Analytical HIC, DSF, cIEF, kD-DLS, and A280 assays were performed as described in Example 1.

cGE/LabChip

Capillary gel electrophoresis was performed on a LabChip® GXII Touch™ Protein Characterization System (Revvity, Waltham, MA). The method was carried out under non-reducing conditions according to the manufacturer's protocol for the Protein Clear HR Assay.

### LC-MS

Prior to analysis, each protein sample (20-25 uL) was treated with PNGase F (New England Biolabs, Inc: P0704S) and incubated for 4-24 h at 37° C. 3 micrograms of each sample was injected onto an Agilent 1290 HPLC instrument equipped with a PLRP-S column (Agilent: PL-1912-1502) operating at a flow rate of 0.3 mL/min. Protein was separated using a linear gradient of water and acetonitrile, each with 0.1% formic acid, and injected into the Agilent 6545XT Q-TOF. Source conditions are as follows: gas temp 350° C., drying gas 12 L/min, nebulizer pressure 60 psi, sheath gas temp 400° C., sheath gas flow 11 L/min, Vcap 5500 V, nozzle volage 2000 V, fragmentor voltage 380 V, and skimmer voltage 140 V. Data was deconvoluted and analyzed using Protein Metrics software.

### Cell Binding Response (MFI)

Chinese hamster ovary (CHO) cells overexpressing human NKG2D or the parental CHO line were allocated at 50,000 cells per well of Falcon 96 well plates. The antibodies were incubated at 1 ug/mL in a 50 uL volume in PBS with 1% bovine serum albumin (BSA) for 1 hour at room temperature with gentle agitation. After the incubation, the cells were centrifuged at 3000 RPM for 1 min and washed three times with PBS+1% BSA with centrifugation steps at each wash step. The cells were stained with 50 μL of a 1:500 dilution factor of anti-human IgG (H+L) conjugated with AlexaFluor 647 in PBS+1% BSA for 30 mins at room temperature with gentle agitation. Following additional wash steps, the cells were re-suspended in 30 μL of PBS+1% BSA and the fluorescence of the AlexaFluor 647 was monitored using an iQue® High-Throughput Screening Cytometer (Sartorius, Fremont, CA) and the median fluorescence intensity (MFI) of the human NKG2D and parental CHO cell lines were determined using the iQue® analysis software.

### Binding Kinetics

Kinetic binding analysis was performed on a Biacore™ T200 or 8K surface plasmon resonance (SPR; Cytiva) with HEPES-buffered saline with 3 mM EDTA and 0.05% tween-20 (HBS-EP+) as the running buffer, unless otherwise noted. A CM5 Series S sensor chip (Cytiva) was immobilized with 50 ug/mL streptavidin (ThermoFisher) in sodium acetate pH 4.5 (Cytiva, Marlborough, MA) using amine coupling to a final density of 2200-2300 response units (RU). CaptureSelect™ Human Fab-kappa Kinetics biotin (ThermoFisher) was captured to the streptavidin surface at 2 ug/mL for 3 mins to a final density of 2000-2500 RU. Multi-cycle kinetics was performed with each antibody and a concentration series of human and cynomolgus IL4, IL13 and NKG2D antigens with regeneration after each antigen injection. The antibodies were captured at 0.5-3 ug/mL depending on format for 60 sec at a 10 uL/min flow rate to a density of 50-200 RU. A 1:3 serial dilution in HBS-EP+ of IL4 starting at 5 nM, IL13 starting at 33.3 nM or NKG2D starting at 100 nM was prepared for a total of 8 concentrations, including a 0 nM injection for double referencing. Each injection of antigen was performed for an association time of 5 mins and a dissociation time of 60 mins for IL4 and 15 mins for IL13 and NKG2D at a flow rate of 30 uL/min. After each antigen injection, the surface was regenerated with 2 injections of 10 mM glycine pH 1.5 (Cytiva) for 5 sec at 30 uL/min for subsequent antibody capture and antigen kinetics cycles. The data was analyzed in the Biacore™ Insight Evaluation software (Cytiva) using a 1:1 binding model. All data was performed in triplicate unless noted otherwise. All experiments were performed at 37° C.

### Cynomolgus NKG2D ELISA

Biotinylated Cyno NKG2D was coated to streptavidin coated 96 well plates (Fisher) at 1 ug/mL in PBS+1% BSA at a final volume of 50 uL overnight at 4*C. After three washes with PBS+1% BSA, the antibodies were incubated with the plates at 1 ug/mL in PBS+1% BSA in a final volume of 50 uL for 1 hour at room temperature with gentle agitation. After an additional triplicate wash, the wells were treated with 50 μL of anti-human IgG (H+L) antibody coupled with horseradish peroxidase (HRP) (Jackson Immunoresearch) for 30 mins at room temperature. After washing, 25 μL of HRP substrate was added to each well and the plates developed and quenched according to the manufacturer's protocols (KPL TMP Microwell Perxidase Substrate Sytems). The plates were read for absorbance at 405 nm using an Envision plate reader.

### Blocking Assay

Human NKG2D containing a C-terminal Fc, anti-human IgG (H+L) antibody conjugated to AlexaFluor 647 and the antibodies were mixed to a final concentration of 1.5 ug/mL, 1:500 and 10 ug/mL respectively in a final volume of 50 μL. The mixtures were allowed to reach equilibrium overnight at 4*C with gentle agitation. CHO cells expressing MICA, MICA or the parental line (negative control) were seeded into wells of 96 well plates (Falcon) to a final number of 50,000 per well. The antibody mixtures were incubated with the cells for 1 hour at room temperature for 1 hour, followed by centrifugation at 3000 RPM for 1 min and washing with PBS+1% BSA. A total of three washes were performed with centrifugation between each step. The cells were re-suspended in 30 μL of PBS+1% BSA and the staining by the fluorescence of the AlexaFluor 647 was monitored on an iQue (Sartorius). Blocking antibodies result in the inability of the Fc-tagged NKG2D to interact with MICA or MICB and results in low signal whereas non-blockers.

### Results

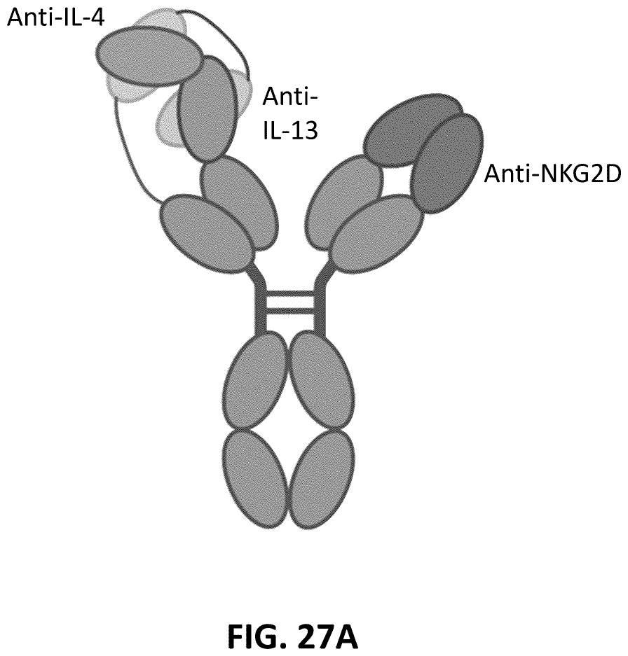

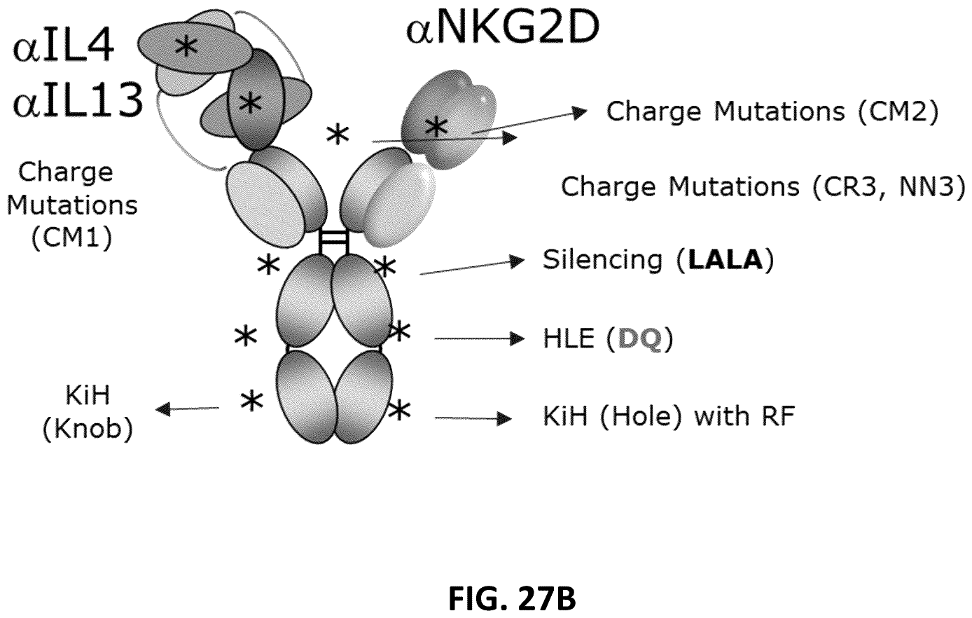

Trispecific binding proteins comprising a cross-over dual variable domain (CODV) arm and a traditional Fab arm were generated and tested for antigen-specific binding. A schematic of the binding protein design is shown in FIG. 27A, with anti-IL-4/anti-IL13 CODV arm as described in Example 1 (see FIG. 1). Mutations were also introduced into a human IgG1 backbone to promote association of the two heavy chains (knobs-into-holes or KiH mutations), to promote purification of the assembled binding protein (H435R/Y436F; RF mutation), to promote proper chain pairing (CM1, CM2, CR3, NN3), to reduce effector function (L234A/L235A; LALA), and to extend half-life (T256D/T307Q; HLE), as shown in FIG. 1C.

For the anti-NKG2D antigen binding domain, various in vivo and in vitro antibody discovery platforms were used, generating over 11,000 potential clones for screening. Multiple platforms were employed due to the complexity of potential functional profiles of anti-NKG2D antigen binding domains, including agonist and antagonist molecules, and the fact that NKG2D-expressing cell lines tend to have low expression and lose expression over time. Clones were characterized for titer, blocking functionality, binding, kinetics, and other functional attributes. Among these, more than 900 unique binders that specifically bound to human and cynomolgus NKG2D were identified. Of these, 274 clones exhibited antagonism or agonism in functional assays. Characteristics of the top 10 anti-NKG2D antigen binding domains are shown in Tables 42 and 43 (tested as monovalent Fab fragments). The top 10 anti-NKG2D antigen binding domains were engineered into trispecific binding proteins as a Fab arm with each of four different IL-4xIL-13 CODV arms according to the structure shown in FIG. 27A for a total of 40 trispecific binding proteins (tested with and without charge mutations, for a total of 80 binding proteins). These 80 trispecific binding proteins were characterized for expression (yield), purity (by SEC analysis), mispairing (by HIC/LC-MS), binding (KD), cross-reactivity with human and cynomolgus NKG2D, potency, and developability (kD-DLS). Binding proteins were expressed from transient transfection of Expi293 cells and purified using a 3-step purification: protein A, anion exchange chromatography, and size exclusion chromatography (SEC).

TABLE 42
Biochemical properties of anti-NKG2D antigen binding domains.

| | | | | | | | Expected | Observed | | | |
|---|---|---|---|---|---|---|---|---|---|---|---|
| | Expression | E280 | | | % | | Mw | Mw | Melting | HIC | HIC |
| Antibody | Titer | (M − 1 | MW | aSEC | Purity | | (mass | (Mass | Temperature | Retention | Area |
| Clone ID | (mg/L) | cm − 1) | (Da) | RT | (aSEC) | pI | Spec) | Spec) | (*C.) | Time (min) | (%) |
| CL-485845 | 13.3 | 76780 | 48008 | 6.513 | 98.29 | | 47964 | | 71.9 | 12.43 | 46.9 |
| CL-600412 | 11.9 | 71280 | 48926 | 6.904 | 100 | 9.80 | 48898 | 48899 | 73.8 | 11.16 | 100.0 |
| CL-607768 | 28.8 | 65320 | 48750 | 6.635 | 99.65 | 9.16 | 48723 | 48724 | | 11.46 | 94.1 |
| CL-485789 | 21.3 | 81250 | 49118 | 6.442 | 96.44 | 7.71 | 49107 | 49021 | 74.6 | 12.27 | 93.1 |
| CL-485797 | 43.7 | 87780 | 48055 | 6.702 | 97.59 | 9.66 | 48010 | 48011 | 77.6 | 14.14 | 89.0 |
| CL-486097 | 7.5 | 84800 | 47893 | 6.454 | 98.04 | 8.92 | 47849 | 48088 | 66.4 | 9.05 | 91.0 |
| CL-524785 | 17.9 | 69790 | 49084 | 6.608 | 98.87 | 9.10 | 49073 | 49074 | 65.2 | 9.67 | 100.0 |
| CL-526201 | 37.0 | 73800 | 48714 | 7.055 | 99.42 | 9.68 | 48686 | 48687 | | 13.14 | 95.0 |
| CL-523781 | 37.7 | 76780 | 48743 | 7.27 | 97.7 | 9.72 | 48715 | 48716 | 70.3 | 12.60 | 95.3 |
| CL-498027 | 37.9 | 74830 | 48050 | 6.426 | 99.24 | 8.66 | 48005 | 48089 | | 8.52 | 29.9 |

TABLE 43
Functional and binding properties of anti-NKG2D antigen binding domains.

| | | | | | | | | | | | | | % |
|---|---|---|---|---|---|---|---|---|---|---|---|---|---|
| | Cyno | | Block- | Cell | | | | | | | | | Inhi- |
| | NKG2D | | ing | Bind- | Cell | | | | | | | % | bi- |
| | Binding, | Block- | Assay, | ing, | Bind- | | | | | | | Inhi- | tion, |
| | ELISA, | ing | MFI of | MFI of | ing, | | | | | | | bi- | yδT |
| Anti- | Absor- | Assay, | CHO- | Parental | MFI of | ka | kd | KD | ka | kd | KD | tion, | Cells |
| body | bance (10 | MFI @ | MICB | CHO @ | hNKG2D | (1/Ms) | (1/s) | (M) | (1/Ms) | (1/s) | (M) | IC50 | (10 |
| Clone | μg/mL | 10 | @ 10 | 10 | @ 10 | his-avi- | his-avi- | his-avi- | his-avi- | his-avi- | his-avi- | (μg/ | μg/ |
| ID | Fab) | μg/mL | μg/mL | μg/mL3 | μg/mL | hNKG2D | hNKG2D | hNKG2D | cNKG2D | cNKG2D | cNKG2D | mL) | mL) |
| CL- | 1.966 | 3418 | 3056 | 1302 | 29107 | 3.06E+03 | 3.83E−03 | 1.25E−06 | 2.22E+04 | 8.66E−03 | 3.90E−07 | 2.947 | 126.8 |
| 485845 | | | | | | | | | | | | | |
| CL- | 2.248 | 3156 | 2732 | 1336 | 51154 | 2.85E+04 | 8.60E−04 | 3.02E−08 | 2.58E+04 | 7.36E−04 | 2.85E−08 | 3.010 | 135.8 |
| 600412 | | | | | | | | | | | | | |
| CL- | 1.705 | 12379 | 37775 | 1044 | 20198 | 7.90E+04 | 5.18E−04 | 6.56E−09 | 1.88E+05 | 6.46E−04 | 3.43E−09 | | |
| 607768 | | | | | | | | | | | | | |
| CL- | 0.996 | 3518 | 3210 | 1348 | 25067 | 5.14E+04 | 2.76E−03 | 5.38E−08 | 5.54E+04 | 2.64E−03 | 4.77E−08 | 6.590 | 74.0 |
| 485789 | | | | | | | | | | | | | |
| CL- | 2.265 | 3243 | 2879 | 1470 | 21960 | 1.93E+04 | 1.20E−02 | 6.23E−07 | 5.13E+04 | 3.03E−02 | 5.91E−07 | 0.450 | 130.3 |
| 485797 | | | | | | | | | | | | | |
| CL- | 2.247 | 3043 | 2927 | 956 | 18167 | 7.84E+03 | 4.51E−03 | 5.76E−07 | 2.17E+04 | 8.37E−03 | 3.85E−07 | 1.395 | 79.4 |
| 486097 | | | | | | | | | | | | | |
| CL- | 2.138 | 3034 | 2679 | 1136 | 23875 | 2.17E+05 | 1.68E−03 | 7.75E−09 | 2.85E+05 | 8.58E−04 | 3.01E−09 | 0.218 | 104.4 |
| 524785 | | | | | | | | | | | | | |
| CL- | 1.728 | 3027 | 3081 | 1335 | 51687 | 7.21E+04 | 7.78E−04 | 1.08E−08 | 8.92E+04 | 8.54E−04 | 9.57E−09 | 0.028 | 159.5 |
| 526201 | | | | | | | | | | | | | |
| CL- | 2.087 | 3135 | 3068 | 1252 | 51560 | 3.64E+04 | 6.67E−04 | 1.83E−08 | 4.16E+04 | 5.61E−04 | 1.35E−08 | 0.264 | 104.8 |
| 523781 | | | | | | | | | | | | | |
| CL- | 2.265 | 2976 | 2704 | 1062 | 19544 | 7.26E+03 | 3.15E−03 | 4.35E−07 | 3.49E+03 | 1.34E−02 | 3.82E−06 | 4.201 | 60.2 |
| 498027 | | | | | | | | | | | | | |

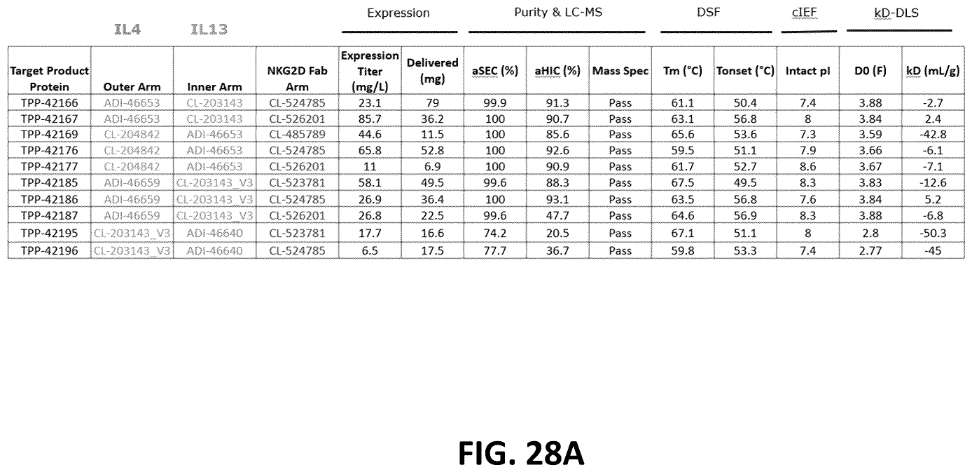

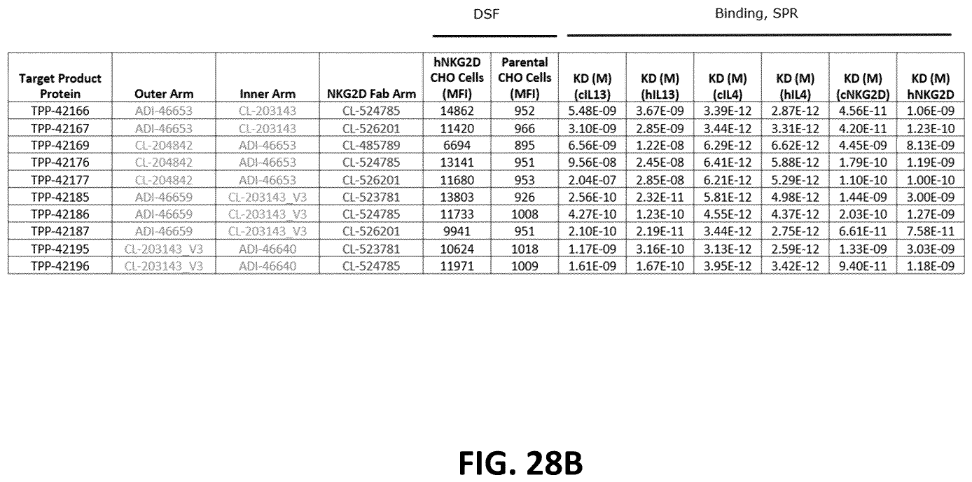

Characterization of the top 10 trispecific binding proteins is described in FIG. 28A. kD-DLS attribute for colloidal stability was performed in 10 mM histidine pH 6.0. Binding of the top 10 trispecific binding proteins to each antigen (human or cyno IL-13, human or cyno IL-4, and human or cyno NKIG2D) as assayed using SPR is shown in FIG. 28B1. These results demonstrate high-affinity, cross-reactive binding (by SPR) for all three antigens, with pM to low nM affinity to all three targets. These results highlighted trispecific binding proteins that bind to hNKG2D-expressing cell lines, bind all three target antigens (both human and cynomolgus), and can simultaneously bind all three targets.

Characterization of binding of trispecific anti-IL-4/IL-1I3/NKIG2D binding proteins to human or cynomolgus NKG2D, IL-4, and IL-13 are shown in Tables 44-46.

TABLE 44
Binding kinetics of trispecific binding proteins to cyno or human NKG2D.

| | | | | | | | Cyno/ |
|---|---|---|---|---|---|---|---|
| | cNKG2D | | | hNKG2D | | | human |
| | ka | kd | KD | ka | kd | KD | ratio |
| Antibody | (1/Ms) | (1/s) | (M) | (1/Ms) | (1/s) | (M) | Ratio |
| IL4 × IL13 × NKG2D TriAb | 4.03E+04 | 2.02E−02 | 5.01E−07 | 3.59E+04 | 2.61E−02 | 7.27E−07 | 0.7 |
| (8D4-8 × BB-13 × 21F2) | | | | | | | |
| (a-IL13_CL-204842_CM1 × | 1.61E+05 | 1.27E−03 | 7.89E−09 | 1.17E+05 | 1.64E−03 | 1.40E−08 | 0.6 |
| a-IL4_ADI-46653_CM1) × | | | | | | | |
| a-KLRK1_CL-485789-CM2- | | | | | | | |
| huIgG1_LALA_CR3_NN3_dK | | | | | | | |
| (a-IL13_CL-204842_CM1 × | 1.21E+05 | 1.43E−06 | 1.18E−11 | 1.01E+05 | 4.19E−05 | 4.15E−10 | 0.0 |
| a-IL4_ADI-46653_CM1) × | | | | | | | |
| a-KLRK1_CL-526201-CM2- | | | | | | | |
| huIgG1_LALA_CR3_NN3_dK | | | | | | | |
| (a-IL4_ADI-46659_CM1 × | 5.61E+04 | 7.53E−05 | 1.34E−09 | 4.74E+04 | 1.53E−04 | 3.23E−09 | 0.4 |
| a-IL13_CL-203143_V3_CM1) × | | | | | | | |
| a-KLRK1_CL-523781-CM2- | | | | | | | |
| huIgG1_LALA_CR3_NN3_dK | | | | | | | |
| (a-IL4_ADI-46659_CM1 × | 2.11E+05 | 7.97E−05 | 3.78E−10 | 1.52E+05 | 3.85E−04 | 2.53E−09 | 0.1 |
| a-IL13_CL-203143_V3_CM1) × | | | | | | | |
| a-KLRK1_CL-524785-CM2- | | | | | | | |
| huIgG1_LALA_CR3_NN3_dK | | | | | | | |
| (a-IL4_ADI-46659_CM1 × | 1.09E+05 | 4.47E−05 | 4.10E−10 | 9.57E+04 | 8.64E−05 | 9.03E−10 | 0.5 |
| a-IL13_CL-203143_V3_CM1) × | | | | | | | |
| a-KLRK1_CL-526201-CM2- | | | | | | | |
| huIgG1_LALA_CR3_NN3_dK | | | | | | | |
| (a-IL13_CL-203143_V3_CM1 × | 5.07E+04 | 6.11E−05 | 1.21E−09 | 4.94E+04 | 1.90E−04 | 3.85E−09 | 0.3 |
| a-IL4_ADI-46640_CM1) × | | | | | | | |
| a-KLRK1_CL-523781-CM2- | | | | | | | |
| huIgG1_LALA_CR3_NN3_dK | | | | | | | |
| (a-IL13_CL-203143_V3_CM1 × | 2.12E+05 | 1.04E−04 | 4.91E−10 | 1.58E+05 | 3.91E−04 | 2.47E−09 | 0.2 |
| a-IL4_ADI-46640_CM1) × | | | | | | | |
| a-KLRK1_CL-524785-CM2- | | | | | | | |
| huIgG1_LALA_CR3_NN3_dK | | | | | | | |
| (a-IL4_ADI-46653_CM1 × | 2.26E+05 | 1.05E−04 | 4.65E−10 | 1.65E+05 | 5.00E−04 | 3.03E−09 | 0.2 |
| a-IL13_CL-203143_CM1) × | | | | | | | |
| a-KLRK1_CL-524785-CM2- | | | | | | | |
| huIgG1_LALA_CR3_NN3_dK | | | | | | | |
| (a-IL4_ADI-46653_CM1 × | 1.26E+05 | 2.80E−05 | 2.22E−10 | 1.13E+05 | 1.23E−05 | 1.09E−10 | 2.0 |
| a-IL13_CL-203143_CM1) × | | | | | | | |
| a-KLRK1_CL-526201-CM2- | | | | | | | |
| huIgG1_LALA_CR3_NN3_dK | | | | | | | |
| (a-IL13_CL-204842_CM1 × | 2.16E+05 | 1.52E−04 | 7.04E−10 | 1.58E+05 | 4.65E−04 | 2.94E−09 | 0.2 |
| a-IL4_ADI-46653_CM1) × | | | | | | | |
| a-KLRK1_CL-524785-CM2- | | | | | | | |
| huIgG1_LALA_CR3_NN3_dK | | | | | | | |

TABLE 45
Binding kinetics of trispecific binding proteins to cyno or human IL-4.

| | cIL4 | | | hIL4 | | | |
|---|---|---|---|---|---|---|---|
| | ka | kd | KD | ka | kd | KD | |
| Antibody | (1/Ms) | (1/s) | (M) | (1/Ms) | (1/s) | (M) | Ratio |
| IL4 × IL13 × NKG2D TriAb | 1.64E+07 | 1.95E−04 | 1.19E−11 | 1.34E+07 | 1.00E−05 | 7.46E−13 | 15.9 |
| (a-IL13_CL-204842_CM1 × a-IL4_ADI- | 3.45E+06 | 4.25E−05 | 1.23E−11 | 3.46E+06 | 1.00E−05 | 2.89E−12 | 4.3 |
| 46653_CM1) × a-KLRK1_CL-485789-CM2- | | | | | | | |
| huIgG1_LALA_CR3_NN3_dK | | | | | | | |
| (a-IL13_CL-204842_CM1 × a-IL4_ADI- | 3.41E+06 | 9.61E−06 | 2.82E−12 | 3.41E+06 | 1.00E−05 | 2.93E−12 | 1.0 |
| 46653_CM1) × a-KLRK1_CL-526201-CM2- | | | | | | | |
| huIgG1_LALA_CR3_NN3_dK | | | | | | | |
| (a-IL4_ADI-46659_CM1 × a-IL13_CL- | 6.05E+06 | 8.61E−06 | 1.42E−12 | 3.68E+06 | 1.00E−05 | 2.72E−12 | 0.5 |
| 203143_V3_CM1) × a-KLRK1_CL-523781-CM2- | | | | | | | |
| huIgG1_LALA_CR3_NN3_dK | | | | | | | |
| (a-IL4_ADI-46659_CM1 × a-IL13_CL- | 4.47E+06 | 1.90E−05 | 4.25E−12 | 4.53E+06 | 2.18E−05 | 4.81E−12 | 0.9 |
| 203143_V3_CM1) × a-KLRK1_CL-524785-CM2- | | | | | | | |
| huIgG1_LALA_CR3_NN3_dK | | | | | | | |
| (a-IL4_ADI-46659_CM1 × a-IL13_CL- | 4.43E+06 | 1.92E−05 | 4.33E−12 | 3.42E+06 | 1.00E−05 | 2.92E−12 | 1.5 |
| 203143_V3_CM1) × a-KLRK1_CL-526201-CM2- | | | | | | | |
| huIgG1_LALA_CR3_NN3_dK | | | | | | | |
| (a-IL13_CL-203143_V3_CM1 × a-IL4_ADI- | 3.90E+06 | 1.00E−05 | 2.56E−12 | 4.14E+06 | 1.00E−05 | 2.42E−12 | 1.1 |
| 46640_CM1) × a-KLRK1_CL-523781-CM2- | | | | | | | |
| huIgG1_LALA_CR3_NN3_dK | | | | | | | |
| (a-IL13_CL-203143_V3_CM1 × a-IL4_ADI- | 3.68E+06 | 1.00E−05 | 2.72E−12 | 3.53E+06 | 1.00E−05 | 2.83E−12 | 1.0 |
| 46640_CM1) × a-KLRK1_CL-524785-CM2- | | | | | | | |
| huIgG1_LALA_CR3_NN3_dK | | | | | | | |
| (a-IL4_ADI-46653_CM1 × a-IL13_CL- | 4.27E+06 | 9.42E−06 | 2.21E−12 | 3.71E+06 | 1.00E−05 | 2.70E−12 | 0.8 |
| 203143_CM1) × a-KLRK1_CL-524785-CM2- | | | | | | | |
| huIgG1_LALA_CR3_NN3_dK | | | | | | | |
| (a-IL4_ADI-46653_CM1 × a-IL13_CL- | 4.68E+06 | 1.54E−05 | 3.29E−12 | 3.52E+06 | 1.00E−05 | 2.84E−12 | 1.2 |
| 203143_CM1) × a-KLRK1_CL-526201-CM2- | | | | | | | |
| huIgG1_LALA_CR3_NN3_dK | | | | | | | |
| (a-IL13_CL-204842_CM1 × a-IL4_ADI- | 3.50E+06 | 1.16E−05 | 3.31E−12 | 3.13E+06 | 1.00E−05 | 3.19E−12 | 1.0 |
| 46653_CM1) × a-KLRK1_CL-524785-CM2- | | | | | | | |
| huIgG1_LALA_CR3_NN3_dK | | | | | | | |

TABLE 46
Binding kinetics of trispecific binding proteins to cyno or human IL-13.

| | cIL13 | | | hIL13 | | | |
|---|---|---|---|---|---|---|---|
| | ka | kd | KD | ka | kd | KD | |
| Antibody | (1/Ms) | (1/s) | (M) | (1/Ms) | (1/s) | (M) | Ratio |
| IL4 × IL13 × NKG2D TriAb | 5.29E+05 | 7.04E−04 | 1.33E−09 | 2.62E+05 | 2.59E−04 | 9.89E−10 | 1.3 |
| (a-IL13_CL-204842_CM1 × a-IL4_ADI- | 8.84E+04 | 1.06E−03 | 1.20E−08 | 1.13E+05 | 1.06E−03 | 9.38E−09 | 1.3 |
| 46653_CM1) × a-KLRK1_CL-485789- | | | | | | | |
| CM2-huIgG1_LALA_CR3_NN3_dK | | | | | | | |
| (a-IL13_CL-204842_CM1 × a-IL4_ADI- | 1.48E+05 | 2.86E−03 | 1.93E−08 | 7.35E+04 | 1.24E−03 | 1.69E−08 | 1.1 |
| 46653_CM1) × a-KLRK1_CL-526201- | | | | | | | |
| CM2-huIgG1_LALA_CR3_NN3_dK | | | | | | | |
| (a-IL4_ADI-46659_CM1 × a-IL13_CL- | 3.13E+05 | 4.03E−04 | 1.29E−09 | 3.04E+05 | 1.59E−04 | 5.23E−10 | 2.5 |
| 203143_V3_CM1) × a-KLRK1_CL-523781- | | | | | | | |
| CM2-huIgG1_LALA_CR3_NN3_dK | | | | | | | |
| (a-IL4_ADI-46659_CM1 × a-IL13_CL- | 2.95E+05 | 4.20E−04 | 1.42E−09 | 2.87E+05 | 1.63E−04 | 5.68E−10 | 2.5 |
| 203143_V3_CM1) × a-KLRK1_CL-524785- | | | | | | | |
| CM2-huIgG1_LALA_CR3_NN3_dK | | | | | | | |
| (a-IL4_ADI-46659_CM1 × a-IL13_CL- | 3.16E+05 | 3.26E−04 | 1.03E−09 | 2.89E+05 | 1.60E−04 | 5.54E−10 | 1.9 |
| 203143_V3_CM1) × a-KLRK1_CL-526201- | | | | | | | |
| CM2-huIgG1_LALA_CR3_NN3_dK | | | | | | | |
| (a-IL13_CL-203143_V3_CM1 × a-IL4_ADI- | 1.84E+05 | 6.07E−04 | 3.30E−09 | 1.56E+05 | 2.60E−04 | 1.67E−09 | 2.0 |
| 46640_CM1) × a-KLRK1_CL-523781- | | | | | | | |
| CM2-huIgG1_LALA_CR3_NN3_dK | | | | | | | |
| (a-IL13_CL-203143_V3_CM1 × a-IL4_ADI- | 2.24E+05 | 4.40E−04 | 1.96E−09 | 2.57E+05 | 1.21E−04 | 4.71E−10 | 4.2 |
| 46640_CM1) × a-KLRK1_CL-524785- | | | | | | | |
| CM2-huIgG1_LALA_CR3_NN3_dK | | | | | | | |
| (a-IL4_ADI-46653_CM1 × a-IL13_CL- | 2.28E+05 | 1.34E−03 | 5.88E−09 | 2.58E+05 | 8.82E−04 | 3.42E−09 | 1.7 |
| 203143_CM1) × a-KLRK1_CL-524785- | | | | | | | |
| CM2-huIgG1_LALA_CR3_NN3_dK | | | | | | | |
| (a-IL4_ADI-46653_CM1 × a-IL13_CL- | 3.02E+05 | 1.58E−03 | 5.23E−09 | 3.22E+05 | 1.02E−03 | 3.17E−09 | 1.7 |
| 203143_CM1) × a-KLRK1_CL-526201- | | | | | | | |
| CM2-huIgG1_LALA_CR3_NN3_dK | | | | | | | |
| (a-IL13_CL-204842_CM1 × a-IL4_ADI- | 7.35E+04 | 2.33E−03 | 3.17E−08 | 8.44E+04 | 1.36E−03 | 1.61E−08 | 2.0 |
| 46653_CM1) × a-KLRK1_CL-524785- | | | | | | | |
| CM2-huIgG1_LALA_CR3_NN3_dK | | | | | | | |

Trispecific anti-IL-4/IL-13/NKG2D binding proteins were also tested for dual binding to IL-4 as well as IL-13 or NKG2D. Briefly, dual binding analysis was performed on a Biacore 8K (Cytiva) with HEPES-buffered saline with 3 mM EDTA and 0.05% tween-20 (HBS-EP+) as the running buffer, unless otherwise noted. A CAP Series S sensor chip (Cytiva) was immobilized with 50 ug/mL CAPture reagent to a final density of 2200-2500 response units (RU). Biotinylated IL4 was captured at 200 nM for 60 sec at 30 uL/min to a final density of 500 RU. The trispecific antibodies were then captured to the immobilized IL4 at 200 nM for 60 sec at 10 uL/min to a final density of 200-600 RU (triAb-dependent). Multi-cycle kinetics was performed with each antibody and a concentration series of human IL13 or NKG2D antigens with regeneration after each antigen injection. A 1:3 serial dilution in HBS-EP+ of IL13 or NKG2D starting at 100 nM was prepared for a total of 4 concentrations, including a 0 nM injection for double referencing. Each injection of antigen was performed for an association time of 5 mins and a dissociation time of 15 mins flow rate of 30 uL/min.

Trispecific anti-IL-4/IL-13/NKG2D binding proteins were also tested for triple binding to IL-4, then IL-13, then NKG2D, or for binding to IL-4, then NKG2D, then IL-13. Briefly, triple antigen binding analysis was performed on a Biacore 8K (Cytiva) with HEPES-buffered saline with 3 mM EDTA and 0.05% tween-20 (HBS-EP+) as the running buffer, unless otherwise noted. A CAP Series S sensor chip (Cytiva) was immobilized with 50 ug/mL CAPture reagent to a final density of 2200-2500 response units (RU). Biotinylated IL4 was captured at 200 nM for 60 sec at 30 uL/min to a final density of 500 RU. The trispecific antibodies were then captured to the immobilized IL4 at 200 nM for 60 sec at 10 uL/min to a final density of 200-600 RU (triAb-dependent). Dual injections were performed with IL13 (Antigen 2) followed by NKG2D (Antigen 3) or NKG2D (antigen 2) followed by IL13 (antigen 3) with regeneration after each cycle. Antigen 2 is injected at 50 nM for 300 sec, followed by a 1:3 serial dilution of antigen three starting at 100 nM for a total of 4 concentrations. Each antigen 3 injection was performed for an association time of 5 mins and a dissociation time of 15 mins flow rate of 30 uL/min. After each cycle, the surface was regenerated with 2 injections of 6 M guanidine hydrochloride with 250 mM sodium hydroxide (Cytiva) for 60 sec at 30 uL/min for subsequent antibody capture and antigen kinetics cycles. The data was analyzed in the Biacore Insight Evaluation software (Cytiva).

Trispecific anti-IL-4/IL-13/NKG2D binding proteins were also tested using kD-DLS. The results are shown in Table 47. Negative or neutral (close to zero) kD indicated attraction and predicted poor high concentration solution behavior. Positive kD values indicated more favorable predicted behavior.

TABLE 47
Characterization of trispecific anti-IL-4/IL-13/NKG2D binding proteins by kD-DLS at 20° C.

| | | | NKG2D | D0 | k_{D} | k_{D} |
|---|---|---|---|---|---|---|
| Binding protein | Outer Arm | Inner Arm | Arm | (F) | (mL/g) | Error± |
| (a-IL4_ADI-46653_CM1 × a- | ADI- | CL- | CL-524785- | 3.88 | −2.7 | 2.4 |
| IL13_CL-203143_CM1) × a- | 46653_CM1 | 203143_CM1 | CM2 | | | |
| KLRK1_CL-524785-CM2- | | | | | | |
| huIgG1_LALA_CR3_NN3_dK | | | | | | |
| (a-IL4_ADI-46653_CM1 × a- | ADI- | CL- | CL-526201- | 3.84 | 2.4 | 1.1 |
| IL13_CL-203143_CM1) × a- | 46653_CM1 | 203143_CM1 | CM2 | | | |
| KLRK1_CL-526201-CM2- | | | | | | |
| huIgG1_LALA_CR3_NN3_dK | | | | | | |
| (a-IL13_CL-204842_CM1 × a- | CL- | ADI- | CL-485789- | 3.59 | −42.8 | 2.4 |
| IL4_ADI-46653_CM1) × a- | 204842_CM1 | 46653_CM1 | CM2 | | | |
| KLRK1_CL-485789-CM2- | | | | | | |
| huIgG1_LALA_CR3_NN3_dK | | | | | | |
| (a-IL13_CL-204842_CM1 × a- | CL- | ADI- | CL-524785- | 3.66 | −6.1 | 1.5 |
| IL4_ADI-46653_CM1) × a- | 204842_CM1 | 46653_CM1 | CM2 | | | |
| KLRK1_CL-524785-CM2- | | | | | | |
| huIgG1_LALA_CR3_NN3_dK | | | | | | |
| (a-IL13_CL-204842_CM1 × a- | CL- | ADI- | CL-526201- | 3.67 | −7.1 | 0.82 |
| IL4_ADI-46653_CM1) × a- | 204842_CM1 | 46653_CM1 | CM2 | | | |
| KLRK1_CL-526201-CM2- | | | | | | |
| huIgG1_LALA_CR3_NN3_dK | | | | | | |
| (a-IL4_ADI-46659_CM1 × a- | ADI- | CL- | CL-523781- | 3.83 | −12.6 | 2.0 |
| IL13_CL-203143_V3_CM1) × a- | 46659_CM1 | 203143_V3_{—} | CM2 | | | |
| KLRK1_CL-523781-CM2- | | CM1 | | | | |
| huIgG1_LALA_CR3_NN3_dK | | | | | | |
| (a-IL4_ADI-46659_CM1 × a- | ADI- | CL- | CL-526201- | 3.88 | −6.8 | 1.4 |
| IL13_CL-203143_V3_CM1) × a- | 46659_CM1 | 203143_V3_{—} | CM2 | | | |
| KLRK1_CL-526201-CM2- | | CM1 | | | | |
| huIgG1_LALA_CR3_NN3_dK | | | | | | |
| (a-IL4_ADI-46659_CM1 × a- | ADI- | CL- | CL-524785- | 3.84 | 5.2 | 1.3 |
| IL13_CL-203143_V3_CM1) × a- | 46659_CM1 | 203143_V3_{—} | CM2 | | | |
| KLRK1_CL-524785-CM2- | | CM1 | | | | |
| huIgG1_LALA_CR3_NN3_dK | | | | | | |
| (a-IL13_CL-203143_V3_CM1 × | CL- | ADI- | CL-523781- | 2.80 | −50.3 | 4.1 |
| a-IL4_ADI-46640_CM1) × a- | 203143_V3_{—} | 46640_CM1 | CM2 | | | |
| KLRK1_CL-523781-CM2- | CM1 | | | | | |
| huIgG1_LALA_CR3_NN3_dK | | | | | | |
| (a-IL13_CL-203143_V3_CM1 × | CL- | ADI- | CL-524785- | 2.77 | −45.0 | 2.5 |
| a-IL4_ADI-46640_CM1) × a- | 203143_V3_{—} | 46640_CM1 | CM2 | | | |
| KLRK1_CL-524785-CM2- | CM1 | | | | | |
| huIgG1_LALA_CR3_NN3_dK | | | | | | |

### Example 10: Crystal Structures of NKG2D in Complex with Newly Generated Anti-NKG2D Antigen Binding Domains

### Materials and Methods

Co-complexes of individual Fabs in complex with Human NKG2D (Acrobiosystems: NKD-H5245) were prepared prior to crystallization using sitting drop vapor diffusion method. All X-ray data sets were collected at SSRF BL19U1 beamline at a wavelength of 0.97853 Å using a Pilatus3 6M detector. The X-ray data was processed using XDS and the phase problem was solved by molecular replacement using the program Phaser. The models were refined using the program Phenix.refine by iteratively building the model in COOT until the refinement converged. Model was validated using Molprobity prior to analyzing the epitope/paratope interactions in the software package MoE.

### Results

The structures of NKG2D in complex with Fabs CL-523781, CL-526201, and CL-524785 were resolved using X-ray crystallography to a resolution of 2.6 A, 2.8 Å and 2.9 Å respectively (FIGS. 29A-29C). NKG2D exists as a dimer in solution, and all the structures displayed a stoichiometry of 2:2 for NKG2D:Fab complexes blocking the receptor binding site on the concave surface.

CL-523781 and NKG2D ligand ULBP6 epitopes overlapped partially (FIG. 29D). However binding of two Fabs to NKG2D dimer would sterically hinder the binding of natural ligands to NKG2D. These antigen binding domains bound to similar epitopes as MICA/MICB and sterically blocked these ligands from binding NKG2D (FIG. 29G). CL-524785 Fab binding to native NKG2D caused steric clashes and disrupted the native NKG2D dimer. CL-526201 was found to have additional interactions with NKG2D in CDR-H3, and the loop conformation was disrupted in CL-526201 by changes in hydrogen bonding, compared to the other Fabs. Interactions between Tyr107 and Tyr36, Gln89 in CL-526201 stabilize the H-bond between Arg103 and Asp188, whereas the Tyr107 is substituted by Phe in CL-523781. Epitopes of each Fab were mapped onto the NKG2D dimer (FIG. 29E). Paratopes of each Fab were also determined as described above (FIG. 29F).

These results were in agreement with epitope binning and in vitro blocking assays. The anti-NKG2D antigen binding domains bound at one Fab per one NKG2D monomer. In conclusion, the symmetric NKG2D dimer was found to bind symmetrically in a 1:1 stoichiometry to the Fab molecules (in contrast to asymmetric binding of NKG2D to its natural ligands in a 2:1 stoichiometry). NKG2D-Fab interactions (mixture of H-bond and hydrophobic) were found in CDRH2, CDRH3 and CDRL3 spread over pockets of residues, mainly in the stirrup loop. The footprint of the natural ligand and Fab partly overlap (the natural ligand interaction sites for ULBP6 and MICA/B are conserved).

Sequences from FIGS. 29E & 29F

Epitopes
(SEQ ID NO: 255)
TESYCGPCPKNWICYKNNCYQFFDESKNWYESQASCMSQNASLLKVYSKEDQDLLKLVKSY
HWMGLVHIPTNGSWQWEDGSILSPNLLTIIEMQKGDCALYASSFKGYIENCSTPNTYICMQRT
Paratopes
CL523781 Heavy chain
(SEQ ID NO: 256)
QLQLQESGPGLVKPSETLSLTCTVSGGSISGSSYYWVWIRQPPGKGLEWIGNIYSSGSTYYNPS
FKSRVIISVDTSKSQFSLKLNSVTAADTAVYYCAKPLLRDYYFYGVDVWGQGTTVTVSSAST
KGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTHHHHHH
CL523781 Light chain
(SEQ ID NO: 257)
DIQLTPSPSFLSASVGDRVTITCRASQAISSYLAWYQQKPGKAPKLLISPASTLQSGVPSRFSGS
GSGTEFTLTISSLQPEDFATYYCQQLKSYPWTFGQGTKVEIKRTVAAPSVFIFPPSDEQLKSGT
ASVVCLLNNFYPREAKVQWKVDNALQSGNSQESVTEQDSKDSTYSLSSTLTLSKADYEKH
KVYACEVTHQGLSSPVTKSFNRGEC
CL526201 Heavy chain
(SEQ ID NO: 258)
QLQLQESGPGLVKPSETLSLTCTVSGGSISSSSYYWVWIRQSPGKGLEWIGNIYSSGSTHYNPS
LKSRVIISVDTSKNQFSLKLKSVTAADTAVYYCAKPLLRDYYYYGVDVWGQGTTVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTHHHHHH
CL526201 Light chain
(SEQ ID NO: 259)
DIQLTPSPSFLSASVGDRVTITCRASQAISSYLAWYQQKPGKAPKLLISPASTLQSGVPSRFSGS
GSGTEFTLTISSLQPEDFATYFCQQLNSFPWTFGQGTKVEIKRTVAAPSVFIFPPSDEQLKSGT
ASVVCLLNNFYPREAKVQWKVDNALQSGNSQESVTEQDSKDSTYSLSSTLTLSKADYEKH
KVYACEVTHQGLSSPVTKSFNRGEC
CL524785 Heavy chain
(SEQ ID NO: 260)
EVQLLESGGGLVQPGGSLRLSCTASGFTFSSYAMSWVRQAPGKGLEWVSLISESGSSTYYAD
SVKGRFTNSRDDSRNTLYLQMNSLRAEDTAVYYCAKDRWFEGYHSYYGMDVWGQGTTVT
VSSASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTHHHHHH
CL524785 Light chain
(SEQ ID NO: 261)
DIQLTQSPSFLSAFVGDRVTITCRASQGISNNLAWYQQKPGKAPKLLVYATSTLQSGVPSRFS
GSGSGTEFTLTVSSLQPEDFATYYCQQLKSYPSFTFGPGTKVDIKRTVAAPSVFIFPPSDEQLK
SGTASVVCLLNNFYPREAKVQWKVDNALQSGNSQESVTEQDSKDSTYSLSSTLTLSKADYE
KHKVYACEVTHQGLSSPVTKSFNRGEC

Example 11: Engineering and further optimization of trispecific binding proteins that bind IL-4, IL-13, and NKG2D

### Materials and Methods

### SDS-PAGE

5 micrograms of each protein sample was prepared with LDS loading dye (Life Technologies: NP0007) plus NuPAGE reducing buffer for reduced samples (Life technologies: NP009). Samples were loaded onto a 4-12% NuPAGE Bis-Tris mini gel (Life Technologies: NPO321BOX) and run according to the manufacturer's instructions using 1×MES running buffer. Gel was stained by Instant Blue Coomassie stain (AbCam: ab119211).

### LC-MS

Prior to analysis, each protein sample (20-25 uL) was treated with PNGase F (New England Biolabs, Inc: P0704S) and incubated for 4-24 h at 37° C. 3 micrograms of each sample was injected onto an Agilent 1290 HPLC instrument equipped with a PLRP-S column (Agilent: PL-1912-1502) operating at a flow rate of 0.3 mL/min. Protein was separated using a linear gradient of water and acetonitrile, each with 0.1% formic acid, and injected into the Agilent 6545XT AdvanceBio LC/Q-TOF system. Source conditions are as follows: gas temp 350° C., drying gas 12 L/min, nebulizer pressure 60 psi, sheath gas temp 400° C., sheath gas flow 11 L/min, Vcap 5500 V, nozzle volage 2000 V, fragmentor voltage 380 V, and skimmer voltage 140 V. Data was deconvoluted and analyzed using Protein Metrics software.

Analytical Size Exclusion Chromatography (aSEC)

5 micrograms of each protein was analyzed on a TSKgel® SuperSW3000 HPLC column (Tosoh Bioscience: 18675) using a 40 mM sodium phosphate buffer with 200 mM arginine, pH 7.0 mobile phase on an Agilent 1260 HPLC system equipped with A280 detection.

### Analytical Hydrophobic Interaction Chromatography

20 micrograms of each protein was analyzed on a MAbPac™ HIC-10 HPLC column (5 mm, 4.6×250 mm, Thermo Fisher Cat No 088481). Running buffer A: 1.5 M ammonium sulfate, 25 mM sodium phosphate pH 7.0. Buffer B: 25 mM sodium phosphate pH 7.0. The protein was injected onto the column and separated using a linear gradient from 30-100% column B over 11 minutes at a flow rate of 0.6 mL/min.

### Results

Anti-NKG2D antigen binding domains were further optimized. Germline mutations were introduced into the VHand VL domains of each of three anti-NKG2D antigen binding domains (CL-523781, CL-524785, and CL-526201) to reduce potential immunogenicity. Further D74E/G mutations were introduced into CL-526201 to reduce potential H-FR3 isomerization. Further D62E mutation was introduced into CL-524785 to reduce potential H-CDR2 isomerization, and S21A/D/T, and S226A/D/T mutations were further introduced to reduce fragmentation.

Binding of trispecific binding proteins comprising these anti-NKG2D antigen binding domains in addition to selected anti-IL-4 and -IL-13 binding domains were characterized for binding kinetics to human and cynomolgus IL-4, IL-13, and NKG2D using SPR. The results are shown in Tables 50-52. These results demonstrate that all three binding proteins retained high-affinity binding to all three target antigens. In summary, six molecules were created with IL4 binding site being either V2 or V2.4 on ADI-46653 or ADI-46659, IL13 binding site fixed at CL-203143_V3.4 and the NKG2D arm with differing levels of mutations. Optimized means NKG2D binding site included variants on both the NKG2D heavy and light chains and corresponded to a whole number version (i.e. V1). Partial optimized corresponds to the reversion of the NKG2D light sequence back to the original sequences (i.e., no modification on the VL) and corresponds to a 0.5 version (V1.5). This improved mispairing but did not solve the problem. Only TPP-42186 did V1.5 have low mispairing, while everything else was still high compared to WT (“unoptimized”). Because reverting to WT light chain on the NKG2D side helped, all un-necessary mutations were removed, generating the CL-524785_V1.5 (based on TPP-42186 with antigenicity and isomerization mutations on VHonly), CL-524785_VH_D62E (based on TPP-42166, isomerization on VHonly) and CL-526201_VH_D74G (based on TPP-42167, isomerization on VHonly) binding sites.

TABLE 50
Binding of trispecific binding protein (a-IL4_ADI-46659_{—}
D111S-CM1 × a-IL13_CL-203143_V3.4-CM1) × a-KLRK1_{—}
CL-524785_V1.5-CM2_huIgG1_LALA_DQ_CR3_NN3_dK)
to human and cynomologus IL-4, IL-13, and NKG2D.

| | ka (1/Ms) | kd (1/s) | KD (M) | % Rmax |
|---|---|---|---|---|
| cIL13 | 2.18E+05 | 3.64E−04 | 1.67E−09 | 56.1 |
| cIL4 | 1.28E+06 | 1.00E−05 | 7.82E−12 | 135.2 |
| cNKG2D | 1.96E+05 | 1.62E−04 | 8.27E−10 | 131.8 |
| hIL13 | 1.91E+05 | 1.35E−04 | 7.07E−10 | 65.0 |
| hIL4 | 2.67E+06 | 1.00E−05 | 3.75E−12 | 116.3 |
| hNKG2D | 1.57E+05 | 4.18E−04 | 2.67E−09 | 141.4 |

TABLE 51
Binding of trispecific binding protein (a-IL4_ADI-46653_{—}
D111E-CM1 × a-IL13_CL-203143_V3.4-CM1) × a-KLRK1_{—}
CL-524785_D62E-CM2_huIgG1_LALA_DQ_CR3_NN3_dK)
to human and cynomologus IL-4, IL-13, and NKG2D.

| | ka (1/Ms) | kd (1/s) | KD (M) | % Rmax |
|---|---|---|---|---|
| cIL13 | 2.44E+05 | 2.67E−04 | 1.10E−09 | 61.0 |
| cIL4 | 2.29E+06 | 1.00E−05 | 4.37E−12 | 124.1 |
| cNKG2D | 2.52E+05 | 1.23E−04 | 4.89E−10 | 116.2 |
| hIL13 | 2.27E+05 | 1.59E−04 | 7.01E−10 | 68.7 |
| hIL4 | 2.89E+06 | 1.00E−05 | 3.47E−12 | 115.4 |
| hNKG2D | 2.26E+05 | 3.96E−04 | 1.75E−09 | 116.5 |

TABLE 52
Binding of trispecific binding protein (a-IL4_ADI-46653_{—}
D111E-CM1 × a-IL13_CL-203143_V3.4-CM1) × a-KLRK1_{—}
CL-526201_D74G-CM2_huIgG1_LALA_DQ_CR3_NN3_dK)
to human and cynomologus IL-4, IL-13, and NKG2D.

| | ka (1/Ms) | kd (1/s) | KD (M) | % Rmax |
|---|---|---|---|---|
| cIL13 | 2.39E+05 | 3.12E−04 | 1.31E−09 | 64.9 |
| cIL4 | 2.69E+06 | 1.00E−05 | 3.72E−12 | 126.0 |
| cNKG2D | 2.45E+05 | 1.47E−04 | 6.00E−10 | 128.4 |
| hIL13 | 2.07E+05 | 1.84E−04 | 8.87E−10 | 75.6 |
| hIL4 | 1.46E+06 | 1.00E−05 | 6.86E−12 | 130.7 |
| hNKG2D | 2.13E+05 | 1.42E−04 | 6.68E−10 | 140.3 |

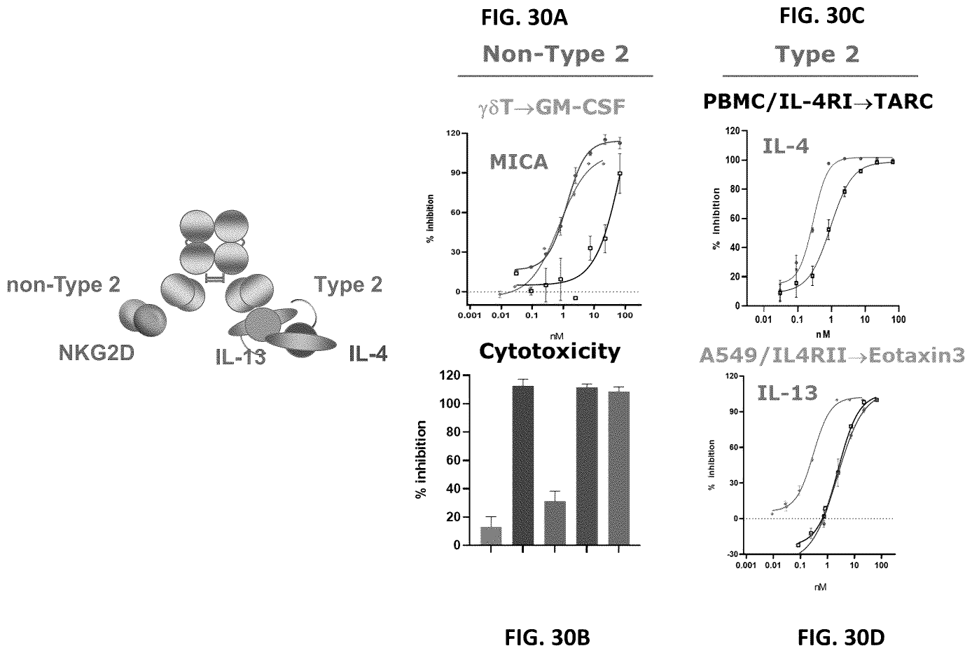

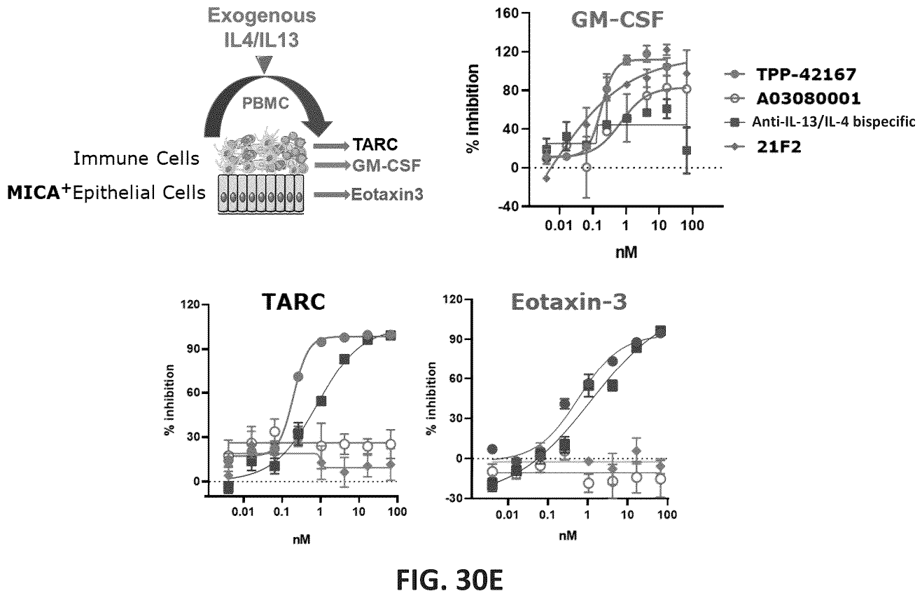

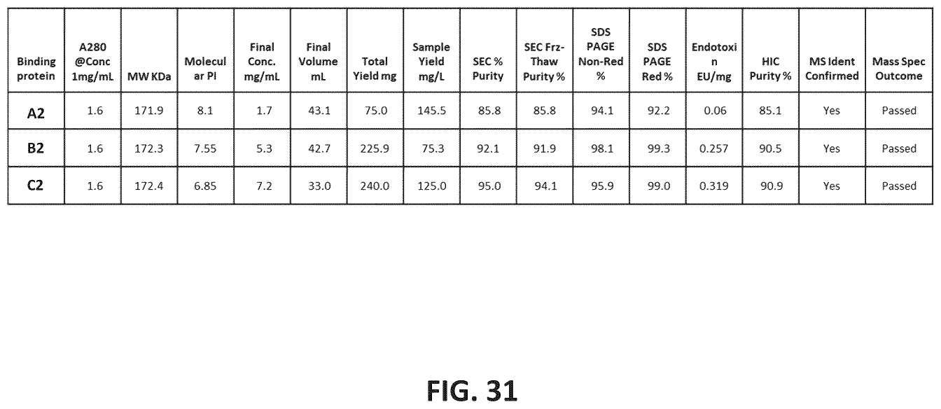

Anti-IL-13/IL-4/NKG2D trispecific binding proteins were also tested for in vitro pharmacological properties. Two trispecific binding proteins were tested for ability to inhibit individual functions of NKG2D, IL-4, and IL-13, as compared to anti-NKG2D mAb or anti-IL-13/IL-4 bispecific antibody (FIGS. 30A-30D and Table 53). Briefly, primary PBMCs were first incubated in the AIM-V medium at 37° C. for 2 hours, and the suspended cells were collected and then primed with IL-15 overnight before proceeding to functional assays (FIGS. 30A & 30B). IL-15-primed PBMCs (FIG. 30A) or primary PBMCs (FIGS. 30C & 30D) were stimulated with plate-bound MICA-IgFc fusion protein (FIG. 30A), or IL-4 or IL-13 (FIGS. 30C & 30D) in the presence or absence of indicated antibody at a dose dependent manner for 2 days before collection of supernatants. Amounts of secreted TARC in the supernatant were measured using ELISA with chemoluminescence detection (MesoScale Discovery). For cytotoxicity assay, IL-15-primed PBMCs were mixed with A549 cells labeled with BATDA (DELFIA EuTDA cytotoxicity kit, PerkinElmer) at a ratio (20:1) in the presence or absence of indicated antibody. After incubation for 2 hr at 37° C., collected supernatants were added with DELFIA europium solution in the DELFIA microtitration plates and shake for 15 min at room temperature before analysis using Read time-resolved fluorometer (Ex320-340, Em615). These results demonstrated that the anti-IL-13/IL-4/NKG2D trispecific binding proteins exhibited good potency for inhibition of individual functions of NKG2D, IL-4, and IL-13.

TABLE 53
Inhibition of IL-4, IL-13, and NKG2D functions by trispecific binding proteins,
as compared to monospecific anti-NKG2D or bispecific anti-IL-13/IL-4 antibody.

| | | non-type 2 | | | |
|---|---|---|---|---|---|
| | | Type-2 | | | |
| | IC50 nM | | | | |
| | NKG2D | IL-4 | | IL-13 | |
| TriAb/BiNb ID | GMCSF | TARC | CCL26 | TARC | CCL26 |
| a-IL4_ADI-46659_D111S-CM1 × | 1.45 | 0.52 | 0.21 | 0.08 | 0.28 |
| a-IL13_CL-203143_V3.4- | | | | | |
| CM1) × a-KLRK1_CL- | | | | | |
| 524785_V1.5- | | | | | |
| CM2_huIgG1_LALA_DQ_CR3_{—} | | | | | |
| NN3_dK | | | | | |
| a-IL4_ADI-46653_D111E-CM1 × | 2.53 | 0.32 | 0.38 | 0.27 | 0.31 |
| a-IL13_CL-203143_V3.4-CM1) × | | | | | |
| a-KLRK1_CL-524785_D62E- | | | | | |
| CM2_huIgG1_LALA_DQ_CR3_{—} | | | | | |
| NN3_dK | | | | | |
| Anti-NKG2D mAb | 0.1 | N/A | N/A | N/A | N/A |
| Anti-IL-13/IL-4 bispecific | N/A | 3.07 | 0.25 | 0.29 | 0.19 |

Trispecific binding protein was tested for ability to inhibit exogenous IL-13 and IL-4 stimulation of PBMCs in the presence of MICA+A549 cells, as compared to anti-NKG2D mAb, anti-IL-13mAb, anti-IL-13/IL-4 mAb, or anti-IL-13/IL-4 bispecific antibody (FIG. 30E and Table 54). Briefly, primary PBMCs were first incubated in the AIM-V medium at 37° C. for 2 hours, and the suspended cells were collected and then primed with IL-15 overnight before proceeding to functional assays. IL-15-primed PBMCs were stimulated with plate-bound MICA-IgFc fusion protein plus IL-4 and IL-13 in the presence or absence of indicated antibody at a dose dependent manner for 2 days before collection of supernatants. Amounts of secreted TARC in the supernatant were measured using ELISA with chemoluminescence detection (MesoScale Discovery). In addition to IL-4 and/or IL-13 inducing TARC production, MIC-A stimulation was found to trigger PBMCs to produce IL-13 that indirectly induced TARC production independent of IL-4. Thus, compared to anti-IL-13/IL-4/NKG2D trispecific binding proteins or anti-IL-13/IL-4 bispecific antibody that inhibited total TARC production (>100%), competitor anti-NKG2D or anti-IL-13 antibody inhibited partial TARC production at a level of 20% or 62%, respectively. Notably, anti-IL-13/IL-4/NKG2D trispecific binding proteins were more potent than anti-IL-13/IL-4 bispecific antibody at inhibiting TARC production induced by MICA/NKG2D, and IL-4/IL-13 pathways. These results demonstrated that the anti-IL-13/IL-4/NKG2D trispecific binding proteins exhibited good potency with full inhibition (>100% top) of total TARC production by combined NKG2D, IL-4, and IL-13 stimulation.

TABLE 54
Inhibition of IL-4, IL-13, and NKG2D functions by trispecific
binding proteins, as compared to anti-NKG2D mAb, anti-
IL-13mAb, or anti-IL-13/IL-4 bispecific antibody.

TARC

| TriAb ID | Top | IC50 nM |
|---|---|---|
| (a-IL4_ADI-46653_CM1 × a- | 101 | 0.57 |
| IL13_CL-203143_CM1) × a- | | |
| KLRK1_CL-526201-CM2- | | |
| huIgG1_LALA_CR3_NN3_dK | | |
| Anti-NKG2D mAb | 20 | 1.43 |
| Anti-IL-13 mAb | 62 | 0.11 |
| Anti-IL-13/IL-4 bispecific | 104 | 1.9 |

Three trispecific binding proteins, A2 ((a-IL4_VR-62824 x a-IL13_VR-62822) x a-KLRK1_VR-93305_huIgG1_LALA_DQ_CR3_NN3_dK; SEQ ID Nos:242-245 for polypeptide chains I, II, III, and IV, respectively), B2 ((a-IL4 VR-93303 x a-IL13_VR-62822) x a-KLRK1_VR-91186_huIgGl_LALA_DQ_CR3_NN3_dK; SEQ ID Nos:246-249 for polypeptide chains I, II, III, and IV, respectively), and C2 ((a-IL4_VR-82824 x a-IL13_VR-62822) x a-KLRK1_VR-93304_huIgG1_LALA_DQ_CR3_NN3_dK SEQ ID Nos:250-253 for polypeptide chains I, II, III, and IV, respectively), were stably produced at large scale. All three binding proteins comprised the anti-IL-13 antigen binding domain CL-203143_V3.4. Binding proteins A2 and C2 comprised the anti-IL-4 antigen binding domain ADI-46653_D111E, while binding protein B2 comprised the anti-IL-4 antigen binding domain ADI-46659_D111S. All binding proteins had the trispecific CODV structure shown in FIG. 27A with the anti-IL-4 antigen binding domain in the outer position of the CODV arm, the anti-IL-13 antigen binding domain in the inner position of the CODV arm, and the anti-NKG2D antigen binding domain in the Fab arm. Binding protein A2 comprised the anti-NKG2D antigen binding domain CL-526201_D74G; binding protein B2 comprised the anti-NKG2D antigen binding domain CL-524785_V1.5; and binding protein C2 comprised the anti-NKG2D antigen binding domain CL-524785_D62E. The attributes of each binding protein after purification are shown in FIG. 6.

Binding of trispecific binding proteins to human and cynomolgus polypeptides of each target were also analyzed by SPR using a CM5 chip. Briefly, 5 ug/mL of each antibody was captured by anti-Kappa capture (ThermoFisher) for 60 seconds at 10 μL/min. IL-4 polypeptides were used at 4-fold dilutions from 5 nM to 19.5 pM; IL-13 and NKG2D polypeptides were used at 3-fold dilutions from 33.33 nM to 45 pM. 300 second association times and 2400/120 second (IL-4) or 900s (IL-13 and NKG2D) were used for dissociation times at 30 μL/min. Regeneration was performed using 10 mM glycine pH 1.5 for 30 seconds at 30 μL/min with 30 seconds stabilization. A 1:1 binding model was used for fitting. The results are shown in Table 55.

TABLE 55
Binding of trispecific binding proteins A2, B2, and
C2 to human or cynomolgus IL-4, IL-13, and NKG2D.

| Capture 1 | Analyte 1 | Capture | | | | | |
|---|---|---|---|---|---|---|---|
| Solution | Solution | Level | ka (1/Ms) | kd (1/s) | KD (M) | Rmax (RU) | % Rmax |
| Trispec. | cIL13 | 185.7 | 2.18E+05 | 3.64E−04 | 1.67E−09 | 7.9 | 56.1 |
| Binding | cIL4 | 187.3 | 1.28E+06 | 1.00E−05 | 7.82E−12 | 23.5 | 135.2 |
| protein B2 | cNKG2D | 187.5 | 1.96E+05 | 1.62E−04 | 8.27E−10 | 27.0 | 131.8 |
| | hIL13 | 184.8 | 1.91E+05 | 1.35E−04 | 7.07E−10 | 9.2 | 65.0 |
| | hIL4 | 185.9 | 2.67E+06 | 1.00E−05 | 3.75E−12 | 20.2 | 116.3 |
| | hNKG2D | 186.5 | 1.57E+05 | 4.18E−04 | 2.67E−09 | 28.8 | 141.4 |
| Trispec. | cIL13 | 268.9 | 2.44E+05 | 2.67E−04 | 1.10E−09 | 12.5 | 61.0 |
| Binding | cIL4 | 271.4 | 2.29E+06 | 1.00E−05 | 4.37E−12 | 31.2 | 124.1 |
| protein C2 | cNKG2D | 303.7 | 2.52E+05 | 1.23E−04 | 4.89E−10 | 38.5 | 116.2 |
| | hIL13 | 267.3 | 2.27E+05 | 1.59E−04 | 7.01E−10 | 14.0 | 68.7 |
| | hIL4 | 269.4 | 2.89E+06 | 1.00E−05 | 3.47E−12 | 28.9 | 115.4 |
| | hNKG2D | 301.5 | 2.26E+05 | 3.96E−04 | 1.75E−09 | 38.3 | 116.5 |
| Trispec. | cIL13 | 214.3 | 2.39E+05 | 3.12E−04 | 1.31E−09 | 10.6 | 64.9 |
| Binding | cIL4 | 215.6 | 2.69E+06 | 1.00E−05 | 3.72E−12 | 25.3 | 126.0 |
| protein A2 | cNKG2D | 217.1 | 2.45E+05 | 1.47E−04 | 6.00E−10 | 30.5 | 128.4 |
| | hIL13 | 213.4 | 2.07E+05 | 1.84E−04 | 8.87E−10 | 12.4 | 75.6 |
| | hIL4 | 214.1 | 1.46E+06 | 1.00E−05 | 6.86E−12 | 26.1 | 130.7 |
| | hNKG2D | 215.9 | 2.13E+05 | 1.42E−04 | 6.68E−10 | 33.1 | 140.3 |

Purity and hydrophobicity were also tested using hydrophobic interaction chromatography (HIC). Briefly, a MAbPac™ (Thermo Fisher Scientific) HIC HPLC column was used (4.6 mm×250 mm) with 10 μg load (1 mg/mL) and a gradient of 1.05M to OM ammonium sulfate (Imin) and 25 mM sodium phosphate pH 7.0. Samples of each trispecific binding protein were used in 10 mM sodium acetate pH 5.0. The results are shown in Table 56. The percent area was less than 9500 due to the complexity of the trispecific format, the presence of mispaired species, or the different hydrophobicities of the different variable regions.

TABLE 56
Purity and hydrophobicity of trispecific
binding proteins A2, B2, and C2.

| Trispecific | | | | |
|---|---|---|---|---|
| binding | | | % area main | |
| protein | Outer Arm | Fab Arm | peak | RT |
| A2 | ADI-46653_{—} | CL-526201_{—} | 78.7 | 11.8 |
| | D111E-CM1 | D74G-CM2 | | |
| B2 | ADI-46659_{—} | CL-524785_{—} | 85.0 | 11.1 |
| | D111S-CM1 | V1.5-CM2 | | |
| C2 | ADI-46653_{—} | CL-524785_{—} | 76.8 | 11.1 |
| | D111E-CM1 | D62E-CM2 | | |

Purity was also tested using SEC with mg/mL trispecific binding protein in 10 mM histidine pH 6, PBS as the mobile phase, and by using capillary gel electrophoresis (cGE). The results are shown in Tables 57 and 58. As shown in Table 57, all three trispecific binding proteins were >98% purity by aSEC indicating little to no higher (aggregates) or lower (fragmentation/mispairing) species post purification. As shown in Table 58, all trispecific molecules were below the cutoff of >9500, suggesting the presence of fragments or mispaired species that are not observable by other techniques.

TABLE 57
Purity of trispecific binding proteins A2, B2, and C2 by SEC.

| Trispecific | | | | |
|---|---|---|---|---|
| Binding | | | % area | |
| Protein | Outer Arm | Fab Arm | main peak | RT |
| A2 | ADI-46653_{—} | CL-526201_{—} | 100 | 4.7 |
| | D111E-CM1 | D74G-CM2 | | |
| B2 | ADI-46659_{—} | CL-524785_{—} | 98.3 | 4.6 |
| | D111S-CM1 | V1.5-CM2 | | |
| C2 | ADI-46653_{—} | CL-524785_{—} | 99.3 | 4.5 |
| | D111E-CM1 | D62E-CM2 | | |

TABLE 58
Purity of trispecific binding proteins A2, B2, and C2 by cGE.

| Trispecific | | | |
|---|---|---|---|
| Binding Protein | Outer Arm | Fab Arm | % Purity |
| A2 | ADI-46653_{—} | CL-526201_{—} | 89.7 |
| | D111E-CM1 | D74G-CM2 | |
| B2 | ADI-46659_{—} | CL-524785_{—} | 92.1 |
| | D111S-CM1 | V1.5-CM2 | |
| C2 | ADI-46653_{—} | CL-524785_{—} | 92.0 |
| | D111E-CM1 | D62E-CM2 | |

Particle size and size distribution of the trispecific binding proteins were also analyzed by dynamic light scattering (DLS), and colloidal stability was analyzed by kD-DLS. The results are shown in Tables 59 and 60. As shown in Table 59, all trispecific molecules showed expected diameter, diffusion coefficient and % PdI for a mass of 175 kDa, further suggestive of highly pure antibodies. As shown in Table 60, all three trispeicifc antibodies had a D0 (F) value in the expected range for a molecule of 175 kDa (<4). The neutral kD values predict possible poor high concentration behavior for these molecules.

TABLE 59
Particle size and distribution of trispecific
binding proteins A2, B2, and C2 by DLS.

| Trispecific | | | | Diffusion | |
|---|---|---|---|---|---|
| Binding | | | Diameter | Coefficient | % |
| Protein | Outer Arm | Fab Arm | (nm) | (um2/s) | PdI |
| A2 | ADI-46653_{—} | CL- | 11.1 | 38.5 | 0.07 |
| | D111E-CM1 | 526201_{—} | | | |
| | | D74G- | | | |
| | | CM2 | | | |
| B2 | ADI-46659_{—} | CL- | 11.2 | 38.4 | 0.08 |
| | D111S-CM1 | 524785_{—} | | | |
| | | V1.5-CM2 | | | |
| C2 | ADI-46653_{—} | CL- | 10.6 | 40.5 | 0.04 |
| | D111E-CM1 | 524785_{—} | | | |
| | | D62E-CM2 | | | |

TABLE 60
Colloidal stability of trispecific binding
proteins A2, B2, and C2 by kD-DLS.

| Trispecific | | | | |
|---|---|---|---|---|
| Binding | | | kD | D0 |
| Protein | Outer Arm | Fab Arm | (mL/g) | (F) |
| A2 | ADI-46653_{—} | CL-526201_{—} | 3.7 | 3.63 |
| | D111E-CM1 | D74G-CM2 | | |
| B2 | ADI-46659_{—} | CL-524785_{—} | −3.8 | 3.83 |
| | D111S-CM1 | V1.5-CM2 | | |
| C2 | ADI-46653_{—} | CL-524785_{—} | −0.7 | 3.87 |
| | D111E-CM1 | D62E-CM2 | | |

Isoelectric point (IEP) of the trispecific binding proteins was also analyzed by capillary isoelectric focusing (cIEF). Molecules were TO in pH 5, and the Maurice IgG method was used (0.1 mg/mL). Binding proteins were digested using FabALACTICA@cysteine protease (Genovis). The results are shown in Table 61.

TABLE 61
IEP of trispecific binding proteins A2, B2, and C2 by cIEF.

| Trispecific | | | mAb | | |
|---|---|---|---|---|---|
| Binding | | | | % acidic | % basic |
| Protein | Outer Arm | Fab Arm | pI | species | species |
| A2 | ADI-46653_{—} | CL- | 7.9 | 18.4 | 11.4 |
| | D111E-CM1 | 526201_{—} | | | |
| | | D74G- | | | |
| | | CM2 | | | |
| B2 | ADI-46659_{—} | CL- | 7.6 | 26.2 | 9.8 |
| | D111S-CM1 | 524785_{—} | | | |
| | | V1.5-CM2 | | | |
| C2 | ADI-46653_{—} | CL- | 7.4 | 22.3 | 14.6 |
| | D111E-CM1 | 524785_{—} | | | |
| | | D62E-CM2 | | | |

Thermal stability of the trispecific binding proteins was also analyzed as described above. Molecules were 1 mg/mL in 10 mM histidine pH 6.0. The results are shown in Table 62.

TABLE 62
Thermal stability of trispecific binding proteins.

| Trispecific | | | | | |
|---|---|---|---|---|---|
| Binding | | | | | |
| Protein | Outer Arm | Fab Arm | TM1 | Tonset | TAGG |
| A2 | ADI-46653_{—} | CL-526201_{—} | 62.8 | 54.8 | 66.4 |
| | D111E-CM1 | D74G-CM2 | | | |
| B2 | ADI-46659_{—} | CL-524785_{—} | 63.9 | 56.9 | 70.3 |
| | D111S-CM1 | V1.5-CM2 | | | |
| C2 | ADI-46653_{—} | CL-524785_{—} | 61.5 | 54.8 | 61.8 |
| | D111E-CM1 | D62E-CM2 | | | |

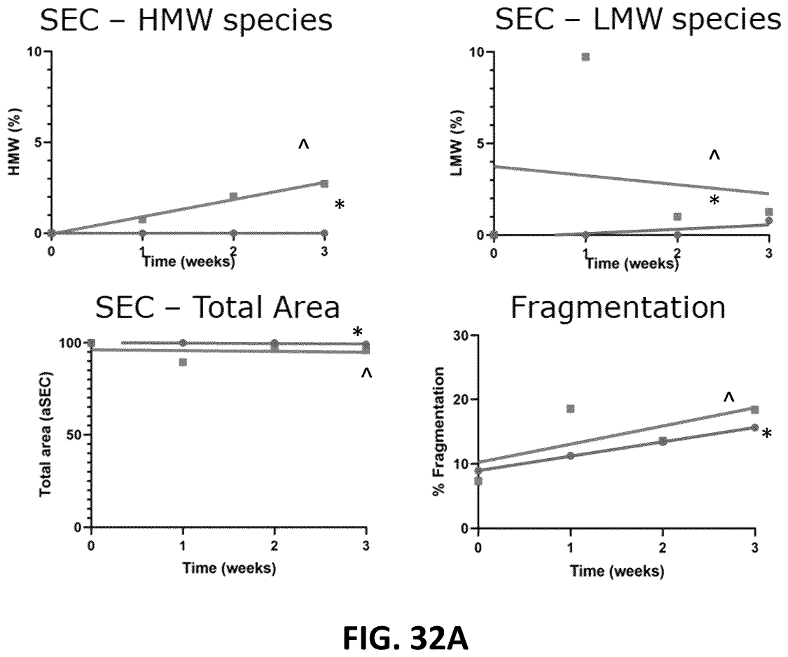

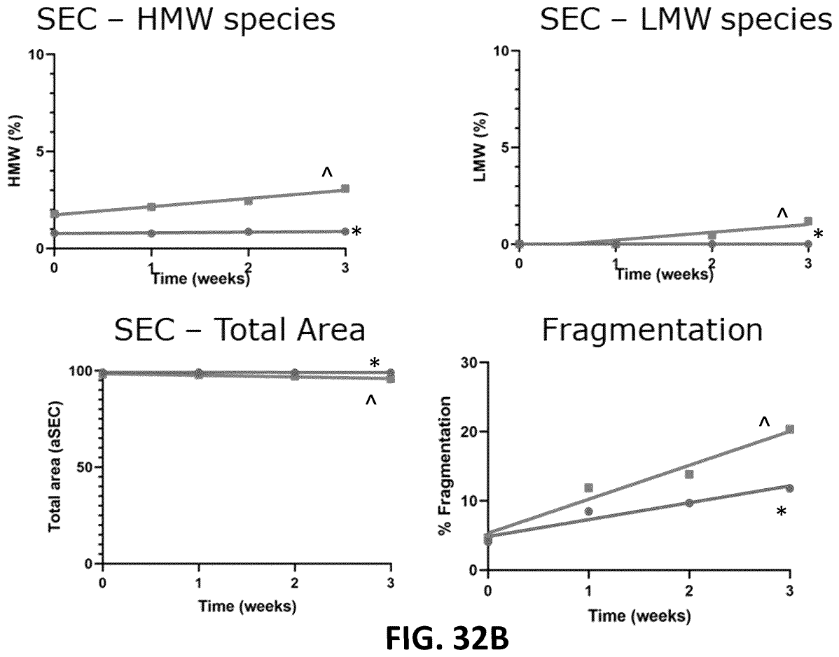

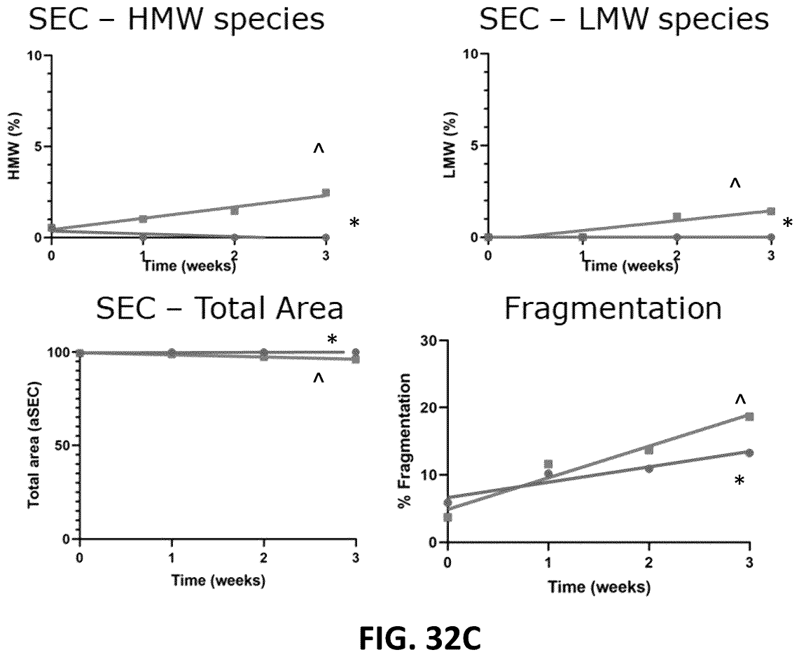

Stability of each binding protein was also analyzed by SEC at pH 5 or pH 8 for 3 weeks at 40° C. The results are shown in FIGS. 32A-32C and Tables 63-69. Active concentration loss per week for each binding protein binding to each human antigen are summarized in Table 30. Upon stress, fragmentation was only observed at high pH for all three antibodies and any sequence modifications, such as oxidation, deamidation or isomerization, did not show trends at low pH stress conditions. Antibody B showed increasing deamidation in the NKG2D light chain at high pH, but this did not impact the active concentration.

TABLE 63
Fragmentation peaks for trispecific binding protein A2 (pH 8).
Fragmentation peaks pH 8

| | |
|---|---|
| 161.9 kDa | 10.1% |
| 149.2 kDa | 4.6% |
| 102.1 kDa | 3.7% |

TABLE 64
Modifications for trispecific binding
protein A2 observed at weeks 0 and 3.

| | | | pH 5 T0 | pH 5 T3 | pH 8 T0 | pH 8 T3 |
|---|---|---|---|---|---|---|
| Protein | Location | Modification | [%] | [%] | [%] | [%] |
| HC1 | M118 | Oxidation | 2.43 | 4.09 | 5.07 | 8.07 |
| HC1 | M237 | Oxidation | 1.01 | 1.83 | 1.27 | 4.85 |

TABLE 65
Fragmentation peaks for trispecific binding protein B2 (pH 8).
Fragmentation peaks pH 8

| | |
|---|---|
| 162.8 kDa | 7.5% |
| 151 kDa | 6.2% |
| 139.3 kDa | 1.4% |
| 105.7 kDa | 1.3% |

TABLE 66
Modifications for trispecific binding
protein B2 observed at weeks 0 and 3.

| | | | pH 5 | pH 5 | pH 8 | pH 8 |
|---|---|---|---|---|---|---|
| | | | T0 | T3 | T0 | T3 |
| Protein | Location | Modification | [%] | [%] | [%] | [%] |
| LC2 | N31/N32 | Deamidation | 7.13 | 7.29 | 7.88 | 46.83 |
| HC1 | W113/M118 | Oxidation | 2.38 | 3.06 | 3.05 | 4.63 |
| HC1 | D104 | Isomerization | 0.76 | 6.04 | 4.74 | 2.94 |
| HC1 | M237 | Oxidation | 0.49 | 1.34 | 0.60 | 4.01 |
| | W240 | | | | | |

TABLE 67
Fragmentation peaks for trispecific binding protein C2 (pH 8).
Fragmentation peaks pH 8

| | |
|---|---|
| 162.2 kDa | 6.0% |
| 150.7 kDa | 6.2% |
| 139.3 kDa | 1.3% |
| 105.9 kDa | 2.2% |

TABLE 68
Modifications for trispecific binding
protein C2 observed at weeks 0 and 3.

| | | | pH 5 | pH 5 | pH 8 | pH 8 |
|---|---|---|---|---|---|---|
| | | | T0 | T3 | T0 | T3 |
| Protein | Location | Modification | [%] | [%] | [%] | [%] |
| HC1 | M118 | Oxidation | 1.97 | 4.04 | 2.86 | 5.44 |
| HC1 | M237 | Oxidation | 0.78 | 1.51 | 0.84 | 3.18 |
| LC2 | N31/32 | Deamidation | 3.67 | 4.03 | 4.77 | 48.58 |
| HC1 | D104/D107 | Isomerization | 1.01 | 2.41 | 1.42 | 4.75 |

TABLE 69
Active concentration losses (per week) for trispecific binding
proteins to each human antigen (mean ± standard deviation).

| Binding protein | pH | hNKG2D | hIL-4 | hIL-13 |
|---|---|---|---|---|
| A2 | 5 | −1.3% ± 0.6 | −1.4% ± 1.5 | −2.2% ± 1.4 |
| | 8 | −2.4% ± 0.8 | −12.5% ± 1.2 | −11.8% ± 1.0 |
| B2 | 5 | −0.4% ± 0.4 | −4.3% ± 0.7 | −3.3% ± 0.8 |
| | 8 | −4.4% ± 0.6 | −1.7% ± 1.6 | −2.3% ± 1.7 |
| C2 | 5 | −0.2% ± 0.9 | −0.6% ± 0.6 | −0.1% ± 0.5 |
| | 8 | −3.0% ± 0.3 | −7.0% ± 1.7 | −6.4% ± 1.7 |

Molecular profiling results for trispecific binding proteins B2 and C2 are shown in Table 70. These experiments revealed expected characteristics for the binding proteins. Isoelectric points and effective charge results for both were below threshold values for risk assessments.

TABLE 70
Molecular profiling of trispecific binding proteins B2 and C2.

| | | Trispecific | Trispecific |
|---|---|---|---|
| | | Binding | Binding |
| | | Protein B2 | Protein C2 |
| SEC-MALLS | Mw (kDa) | 171.7 | 172.2 |
| | HMWS (%) | 2.5 | 3.5 |
| | LMWS (%) | 1.9 | 2.6 |
| | Monomer (%) | 95.6 | 94.2 |
| cIEF | pI | 7.70 | 7.44 |
| | Peak count | 4 | 3 |
| | Main (%) | 55.2 | 58.2 |
| | Acidic (%) | 29.8 | 24.2 |
| | Basic (%) | 15.1 | 17.4 |
| WCX | Peak count | 3 | 2 |
| | Main (%) | 50.8 | 77.7 |
| CZE | z* | 4.60 | 4.16 |
| HIC | Peak count | 1 | 1 |
| | Main (%) | 100 | 100 |
| CD (far UV) | Structure | Ig-like | Ig-like |
| SV-AUC | S | 7.28 | 7.42 |
| | Mw (kDa) | 162.8 | 161.2 |
| | Rh (nm) | 5.17 | 5.02 |
| DLS | Rh (nm) | 5.6 | 5.6 |
| | PD (%) | 2.9 | 3.1 |
| DSC | Tₒₙₛₑₜ (° C.) | 55.9 | 56.7 |
| Intact mass | Mw (D) | 175188 | 175261 |

Manufacturing stress response results for trispecific binding proteins B2 and C2 are shown in Table 71. These experiments revealed good stability overall.

TABLE 71
Manufacturing stress response results for
trispecific binding proteins B2 and C2.

| | | Trispecific | Trispecific |
|---|---|---|---|
| | | Binding | Binding |
| Stress | Assessment | Protein B2 | Protein C2 |
| Reduction | EC50 (mM) | 1.42 | 1.39 |
| Sensitivity | | | |
| Mock VI | DLS (Max % PD) | MM | MM |
| | LabChip (Frag onset) | 3.4 | 3.4 |
| | A280 (Min recovery %) | 94.6 | 94.4 |
| | SEC (Agg onset) | 3.2 | N/A |
| Agitation | A280 (mg · mL/day) | 0.23 | −0.04 |
| | μ-nephelometry (NTU/day) | 0.88 | 0.29 |
| | LabChip (% LMWS/day) | −0.30 | 0.17 |
| | SEC (% HMWS/day) | −0.01 | 0.00 |
| Freeze-Thaw | A280 (mg · mL/cycle) | −0.89 | −0.55 |
| | μ-nephelometry (NTU/cycle) | −0.55 | 0.00 |
| | LabChip (% LMWS/cycle) | −0.14 | −0.06 |
| | SEC (% HMWS/cycle) | −0.02 | −0.02 |

Deviceability results for trispecific binding proteins B2 and C2 are shown in Table 72. These experiments revealed that both binding proteins had kD-DLS values below the risk threshold. Both binding proteins had measured viscosity >20 cP.

TABLE 72
Deviceability of trispecific binding proteins B2 and C2.

| | | Trispecific | Trispecific |
|---|---|---|---|
| | | Binding | Binding |
| Stress | Assessment | Protein B2 | Protein C2 |
| Silicone Oil | μ-nephelometry (NTU/day) | −1.54 | 0.22 |
| Compatibility | LabChip (% LMWS/day) | −0.06 | 0.29 |
| | A280 (mg · mL/day) | 0.00 | −0.01 |
| | SEC (% HMWS/day) | −0.08 | −0.05 |
| High | Concentration (mg/mL) | 151.5 | 147.1 |
| Concentration | Viscosity (cPa) | 51.3 | 43.6 |
| Behavior | μ-nephelometry (NTU) | 9.9 | 13.0 |
| | Visual Inspection | Clear | Clear |
| Predicative Testing | kD-DLS (mL/g, H6.0) | −7.8 | −2.1 |

Overall, minimal risks were observed for both binding proteins with respect to reduction sensitivity, agitation stress, and freeze/thawing. Potential particle formation was observed for both after low pH exposure. Both had isoelectric points <8 and effective charges <6, as well as elevated levels in high molecular weight species. Both showed loss of binding and elevated aggregation in certain buffers. Both showed kD-DLS <20 mL/g and viscosity >20 cP. Both also demonstrated acceptable titer and purity upon large-scale production.

1: A trispecific binding protein comprising four polypeptide chains that form three antigen binding sites, wherein a first polypeptide chain of the binding protein comprises a structure represented by the formula: 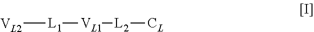
and a second polypeptide chain of the binding protein comprises a structure represented by the formula: 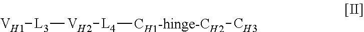
and a third polypeptide chain of the binding protein comprises a structure represented by the formula: 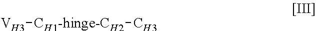
and a fourth polypeptide chain of the binding protein comprises a structure represented by the formula: 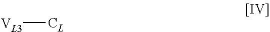
wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are each independently amino acid linkers or zero amino acids in length;
wherein the polypeptide of formula I and the polypeptide of formula II form a cross-over light chain-heavy chain pair of the binding protein;
wherein V_{H1} and V_{L1} form a binding pair and a first antigen binding site, V_{H2} and V_{L2} form a binding pair and a second antigen binding site, and V_{H3} and V_{L3} form a binding pair and a third antigen binding site;
wherein one of the three antigen binding sites specifically binds an interleukin-4 (IL-4) polypeptide, one of the three antigen binding sites specifically binds an interleukin-13 (IL-13) polypeptide, and one of the three antigen binding sites specifically binds an OX40L polypeptide; and
wherein the antigen binding site that specifically binds an OX40L polypeptide comprises a heavy chain variable (VH) domain that comprises a CDR-H1 comprising the amino acid sequence of SEQ ID NO:78, a CDR-H2 comprising the amino acid sequence of SEQ ID NO:79, and a CDR-H3 comprising the amino acid sequence of SEQ ID NO:80, and a light chain variable (VH) domain that comprises a CDR-L1 comprising the amino acid sequence of SEQ ID NO:81, a CDR-L2 comprising the amino acid sequence of SEQ ID NO:82, and a CDR-L3 comprising the amino acid sequence of SEQ ID NO:83;
optionally wherein the antigen binding site that specifically binds the OX40L polypeptide is the third antigen binding site formed by V_{H3} and V_{L3}.

2. (canceled)

3. The binding protein of claim 1, wherein the binding site that specifically binds an IL-4 polypeptide comprises:
(a) a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO: 3, and a VL domain that comprises a CDR-L1 comprising the amino acid sequence SEQ ID NO:6, a CDR-L2 comprising the amino acid sequence of SEQ ID NO:8, and a CDR-L3 comprising the amino acid sequence of SEQ ID NO: 10;
(b) a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11;
(c) a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:4, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11;
(d) a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:5, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11;
(e) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24, and a VH domain that comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10;
(f) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11;
(g) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, and a VH domain that comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10;
(h) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11;
(i) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:26, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11; or
(j) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:27, and a VH domain that comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11;
optionally wherein the binding site that specifically binds an IL-4 polypeptide comprises:
(a) a VH domain that comprises an amino acid sequence that is at least 95%, at least 96%, at least 97%, at least 98%, at least 99%, or 100% identical to the amino acid sequence of SEQ ID NO:34 and a VH domain that comprises an amino acid sequence that is at least 95%, at least 96%, at least 97%, at least 98%, at least 99%, or 100% identical to the amino acid sequence of SEQ ID NO:35, optionally V_{H1} comprises an amino acid sequence that is at least 95%, at least 96%, at least 97%, at least 98%, at least 99%, or 100% identical to the amino acid sequence of SEQ ID NO:34, and V_{L1} comprises an amino acid sequence that is at least 95%, at least 96%, at least 97%, at least 98%, at least 99%, or 100% identical to the amino acid sequence of SEQ ID NO:35;
(b) a VH domain that comprises an amino acid sequence that is at least 95%, at least 96%, at least 97%, at least 98%, at least 99%, or 100% identical to the amino acid sequence of SEQ ID NO:36 and a VH domain that comprises an amino acid sequence that is at least 95%, at least 96%, at least 97%, at least 98%, at least 99%, or 100% identical to the amino acid sequence of SEQ ID NO:37, optionally V_{H1} comprises an amino acid sequence that is at least 95%, at least 96%, at least 97%, at least 98%, at least 99%, or 100% identical to the amino acid sequence of SEQ ID NO:36, and V_{L1} comprises an amino acid sequence that is at least 95%, at least 96%, at least 97%, at least 98%, at least 99%, or 100% identical to the amino acid sequence of SEQ ID NO:37;
(c) a VH domain that comprises an amino acid sequence that is at least 95%, at least 96%, at least 97%, at least 98%, at least 99%, or 100% identical to the amino acid sequence of SEQ ID NO:38 and a VH domain that comprises an amino acid sequence that is at least 95%, at least 96%, at least 97%, at least 98%, at least 99%, or 100% identical to the amino acid sequence of SEQ ID NO:39, optionally V_{H1} comprises an amino acid sequence that is at least 95%, at least 96%, at least 97%, at least 98%, at least 99%, or 100% identical to the amino acid sequence of SEQ ID NO:38, and V_{L1} comprises an amino acid sequence that is at least 95%, at least 96%, at least 97%, at least 98%, at least 99%, or 100% identical to the amino acid sequence of SEQ ID NO:39;
(d) a VH domain that comprises an amino acid sequence that is at least 95%, at least 96%, at least 97%, at least 98%, at least 99%, or 100% identical to the amino acid sequence of SEQ ID NO:40 and a VH domain that comprises an amino acid sequence that is at least 95%, at least 96%, at least 97%, at least 98%, at least 99%, or 100% identical to the amino acid sequence of SEQ ID NO:41, optionally V_{H1} comprises an amino acid sequence that is at least 95%, at least 96%, at least 97%, at least 98%, at least 99%, or 100% identical to the amino acid sequence of SEQ ID NO:40, and V_{L1} comprises an amino acid sequence that is at least 95%, at least 96%, at least 97%, at least 98%, at least 99%, or 100% identical to the amino acid sequence of SEQ ID NO:41;
(e) a VH domain that comprises an amino acid sequence that is at least 95%, at least 96%, at least 97%, at least 98%, at least 99%, or 100% identical to the amino acid sequence of SEQ ID NO:42 and a VH domain that comprises an amino acid sequence that is at least 95%, at least 96%, at least 97%, at least 98%, at least 99%, or 100% identical to the amino acid sequence of SEQ ID NO:43, optionally V_{H1} comprises an amino acid sequence that is at least 95%, at least 96%, at least 97%, at least 98%, at least 99%, or 100% identical to the amino acid sequence of SEQ ID NO:42, and V_{L1} comprises an amino acid sequence that is at least 95%, at least 96%, at least 97%, at least 98%, at least 99%, or 100% identical to the amino acid sequence of SEQ ID NO:43;
(f) a VH domain that comprises an amino acid sequence that is at least 95%, at least 96%, at least 97%, at least 98%, at least 99%, or 100% identical to the amino acid sequence of SEQ ID NO:44 and a VH domain that comprises an amino acid sequence that is at least 95%, at least 96%, at least 97%, at least 98%, at least 99%, or 100% identical to the amino acid sequence of SEQ ID NO:43, optionally V_{H1} comprises an amino acid sequence that is at least 95%, at least 96%, at least 97%, at least 98%, at least 99%, or 100% identical to the amino acid sequence of SEQ ID NO:44, and V_{L1} comprises an amino acid sequence that is at least 95%, at least 96%, at least 97%, at least 98%, at least 99%, or 100% identical to the amino acid sequence of SEQ ID NO:43;
(g) a VH domain that comprises an amino acid sequence that is at least 95%, at least 96%, at least 97%, at least 98%, at least 99%, or 100% identical to the amino acid sequence of SEQ ID NO:45 and a VH domain that comprises an amino acid sequence that is at least 95%, at least 96%, at least 97%, at least 98%, at least 99%, or 100% identical to the amino acid sequence of SEQ ID NO:43, optionally V_{H1} comprises an amino acid sequence that is at least 95%, at least 96%, at least 97%, at least 98%, at least 99%, or 100% identical to the amino acid sequence of SEQ ID NO:45, and V_{L1} comprises an amino acid sequence that is at least 95%, at least 96%, at least 97%, at least 98%, at least 99%, or 100% identical to the amino acid sequence of SEQ ID NO:43;
(h) a VH domain that comprises an amino acid sequence that is at least 95%, at least 96%, at least 97%, at least 98%, at least 99%, or 100% identical to the amino acid sequence of SEQ ID NO:46 and a VH domain that comprises an amino acid sequence that is at least 95%, at least 96%, at least 97%, at least 98%, at least 99%, or 100% identical to the amino acid sequence of SEQ ID NO:47, optionally V_{H1} comprises an amino acid sequence that is at least 95%, at least 96%, at least 97%, at least 98%, at least 99%, or 100% identical to the amino acid sequence of SEQ ID NO:46, and V_{L1} comprises an amino acid sequence that is at least 95%, at least 96%, at least 97%, at least 98%, at least 99%, or 100% identical to the amino acid sequence of SEQ ID NO:47;
(i) a VH domain that comprises an amino acid sequence that is at least 95%, at least 96%, at least 97%, at least 98%, at least 99%, or 100% identical to the amino acid sequence of SEQ ID NO:48 and a VH domain that comprises an amino acid sequence that is at least 95%, at least 96%, at least 97%, at least 98%, at least 99%, or 100% identical to the amino acid sequence of SEQ ID NO:39, optionally V_{H1} comprises an amino acid sequence that is at least 95%, at least 96%, at least 97%, at least 98%, at least 99%, or 100% identical to the amino acid sequence of SEQ ID NO:48, and V_{L1} comprises an amino acid sequence that is at least 95%, at least 96%, at least 97%, at least 98%, at least 99%, or 100% identical to the amino acid sequence of SEQ ID NO:39;
(j) a VH domain that comprises an amino acid sequence that is at least 95%, at least 96%, at least 97%, at least 98%, at least 99%, or 100% identical to the amino acid sequence of SEQ ID NO:49 and a VH domain that comprises an amino acid sequence that is at least 95%, at least 96%, at least 97%, at least 98%, at least 99%, or 100% identical to the amino acid sequence of SEQ ID NO:41, optionally V_{H1} comprises an amino acid sequence that is at least 95%, at least 96%, at least 97%, at least 98%, at least 99%, or 100% identical to the amino acid sequence of SEQ ID NO:49, and V_{L1} comprises an amino acid sequence that is at least 95%, at least 96%, at least 97%, at least 98%, at least 99%, or 100% identical to the amino acid sequence of SEQ ID NO:41;
(k) a VH domain that comprises an amino acid sequence that is at least 95%, at least 96%, at least 97%, at least 98%, at least 99%, or 100% identical to the amino acid sequence of SEQ ID NO:50 and a VH domain that comprises an amino acid sequence that is at least 95%, at least 96%, at least 97%, at least 98%, at least 99%, or 100% identical to the amino acid sequence of SEQ ID NO:43, optionally V_{H1} comprises an amino acid sequence that is at least 95%, at least 96%, at least 97%, at least 98%, at least 99%, or 100% identical to the amino acid sequence of SEQ ID NO:50, and V_{L1} comprises an amino acid sequence that is at least 95%, at least 96%, at least 97%, at least 98%, at least 99%, or 100% identical to the amino acid sequence of SEQ ID NO:43;
(l) a VH domain that comprises an amino acid sequence that is at least 95%, at least 96%, at least 97%, at least 98%, at least 99%, or 100% identical to the amino acid sequence of SEQ ID NO:51 and a VH domain that comprises an amino acid sequence that is at least 95%, at least 96%, at least 97%, at least 98%, at least 99%, or 100% identical to the amino acid sequence of SEQ ID NO:37, optionally V_{H1} comprises an amino acid sequence that is at least 95%, at least 96%, at least 97%, at least 98%, at least 99%, or 100% identical to the amino acid sequence of SEQ ID NO:51, and V_{L1} comprises an amino acid sequence that is at least 95%, at least 96%, at least 97%, at least 98%, at least 99%, or 100% identical to the amino acid sequence of SEQ ID NO:37;
(m) a VH domain that comprises an amino acid sequence that is at least 95%, at least 96%, at least 97%, at least 98%, at least 99%, or 100% identical to the amino acid sequence of SEQ ID NO:52 and a VH domain that comprises an amino acid sequence that is at least 95%, at least 96%, at least 97%, at least 98%, at least 99%, or 100% identical to the amino acid sequence of SEQ ID NO:39, optionally V_{H1} comprises an amino acid sequence that is at least 95%, at least 96%, at least 97%, at least 98%, at least 99%, or 100% identical to the amino acid sequence of SEQ ID NO:52, and V_{L1} comprises an amino acid sequence that is at least 95%, at least 96%, at least 97%, at least 98%, at least 99%, or 100% identical to the amino acid sequence of SEQ ID NO:39;
(n) a VH domain that comprises an amino acid sequence that is at least 95%, at least 96%, at least 97%, at least 98%, at least 99%, or 100% identical to the amino acid sequence of SEQ ID NO:53 and a VH domain that comprises an amino acid sequence that is at least 95%, at least 96%, at least 97%, at least 98%, at least 99%, or 100% identical to the amino acid sequence of SEQ ID NO:41, optionally V_{H1} comprises an amino acid sequence that is at least 95%, at least 96%, at least 97%, at least 98%, at least 99%, or 100% identical to the amino acid sequence of SEQ ID NO:53, and V_{L1} comprises an amino acid sequence that is at least 95%, at least 96%, at least 97%, at least 98%, at least 99%, or 100% identical to the amino acid sequence of SEQ ID NO:41;
(o) a VH domain that comprises an amino acid sequence that is at least 95%, at least 96%, at least 97%, at least 98%, at least 99%, or 100% identical to the amino acid sequence of SEQ ID NO:54 and a VH domain that comprises an amino acid sequence that is at least 95%, at least 96%, at least 97%, at least 98%, at least 99%, or 100% identical to the amino acid sequence of SEQ ID NO:43, optionally V_{H1} comprises an amino acid sequence that is at least 95%, at least 96%, at least 97%, at least 98%, at least 99%, or 100% identical to the amino acid sequence of SEQ ID NO:54, and V_{L1} comprises an amino acid sequence that is at least 95%, at least 96%, at least 97%, at least 98%, at least 99%, or 100% identical to the amino acid sequence of SEQ ID NO:43;
(p) a VH domain that comprises an amino acid sequence that is at least 95%, at least 96%, at least 97%, at least 98%, at least 99%, or 100% identical to the amino acid sequence of SEQ ID NO:55 and a VH domain that comprises an amino acid sequence that is at least 95%, at least 96%, at least 97%, at least 98%, at least 99%, or 100% identical to the amino acid sequence of SEQ ID NO:43, optionally V_{H1} comprises an amino acid sequence that is at least 95%, at least 96%, at least 97%, at least 98%, at least 99%, or 100% identical to the amino acid sequence of SEQ ID NO:55, and V_{L1} comprises an amino acid sequence that is at least 95%, at least 96%, at least 97%, at least 98%, at least 99%, or 100% identical to the amino acid sequence of SEQ ID NO:43; or
(q) a VH domain that comprises an amino acid sequence that is at least 95%, at least 96%, at least 97%, at least 98%, at least 99%, or 100% identical to the amino acid sequence of SEQ ID NO:56 and a VH domain that comprises an amino acid sequence that is at least 95%, at least 96%, at least 97%, at least 98%, at least 99%, or 100% identical to the amino acid sequence of SEQ ID NO:43, optionally V_{H1} comprises an amino acid sequence that is at least 95%, at least 96%, at least 97%, at least 98%, at least 99%, or 100% identical to the amino acid sequence of SEQ ID NO:56, and V_{L1} comprises an amino acid sequence that is at least 95%, at least 96%, at least 97%, at least 98%, at least 99%, or 100% identical to the amino acid sequence of SEQ ID NO:43.

4: The binding protein of claim 1, wherein the binding site that specifically binds an IL-13 polypeptide comprises:
(a) a VH domain that comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, and a VH domain that comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62; or
(b) a VH domain that comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:108, and a VH domain that comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62;
optionally wherein the binding site that specifically binds an IL-13 polypeptide comprises:
(a) a VH domain that comprises an amino acid sequence that is at least 95%, at least 96%, at least 97%, at least 98%, at least 99%, or 100% identical to the amino acid sequence of SEQ ID NO:70 and a VH domain that comprises an amino acid sequence that is at least 95%, at least 96%, at least 97%, at least 98%, at least 99%, or 100% identical to the amino acid sequence of SEQ ID NO:71, optionally V_{H2} comprises an amino acid sequence that is at least 95%, at least 96%, at least 97%, at least 98%, at least 99%, or 100% identical to the amino acid sequence of SEQ ID NO:70, and V_{L2} comprises an amino acid sequence that is at least 95%, at least 96%, at least 97%, at least 98%, at least 99%, or 100% identical to the amino acid sequence of SEQ ID NO:71;
(b) a VH domain that comprises an amino acid sequence that is at least 95%, at least 96%, at least 97%, at least 98%, at least 99%, or 100% identical to the amino acid sequence of SEQ ID NO:72 and a VH domain that comprises an amino acid sequence that is at least 95%, at least 96%, at least 97%, at least 98%, at least 99%, or 100% identical to the amino acid sequence of SEQ ID NO:73, optionally V_{H2} comprises an amino acid sequence that is at least 95%, at least 96%, at least 97%, at least 98%, at least 99%, or 100% identical to the amino acid sequence of SEQ ID NO:72, and V_{L2} comprises an amino acid sequence that is at least 95%, at least 96%, at least 97%, at least 98%, at least 99%, or 100% identical to the amino acid sequence of SEQ ID NO:73;
(c) a VH domain that comprises an amino acid sequence that is at least 95%, at least 96%, at least 97%, at least 98%, at least 99%, or 100% identical to the amino acid sequence of SEQ ID NO:74 and a VH domain that comprises an amino acid sequence that is at least 95%, at least 96%, at least 97%, at least 98%, at least 99%, or 100% identical to the amino acid sequence of SEQ ID NO:75, optionally V_{H2} comprises an amino acid sequence that is at least 95%, at least 96%, at least 97%, at least 98%, at least 99%, or 100% identical to the amino acid sequence of SEQ ID NO:74, and V_{L2} comprises an amino acid sequence that is at least 95%, at least 96%, at least 97%, at least 98%, at least 99%, or 100% identical to the amino acid sequence of SEQ ID NO:75; or
(d) a VH domain that comprises an amino acid sequence that is at least 95%, at least 96%, at least 97%, at least 98%, at least 99%, or 100% identical to the amino acid sequence of SEQ ID NO:76 and a VH domain that comprises an amino acid sequence that is at least 95%, at least 96%, at least 97%, at least 98%, at least 99%, or 100% identical to the amino acid sequence of SEQ ID NO:77, optionally V_{H2} comprises an amino acid sequence that is at least 95%, at least 96%, at least 97%, at least 98%, at least 99%, or 100% identical to the amino acid sequence of SEQ ID NO:76, and V_{L2} comprises an amino acid sequence that is at least 95%, at least 96%, at least 97%, at least 98%, at least 99%, or 100% identical to the amino acid sequence of SEQ ID NO:77.

5: The binding protein of claim 1, wherein V_{H1} comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3; V_{L1} comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10; V_{H2} comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59; and V_{L2} comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62;
optionally wherein V_{H1} comprises the amino acid sequence of SEQ ID NO:34, V_{L1} comprises the amino acid sequence of SEQ ID NO:35, V_{H2} comprises the amino acid sequence of SEQ ID NO:70, and V_{L2} comprises the amino acid sequence of SEQ ID NO:71.

6. (canceled)

7. The binding protein of claim 1, wherein V_{H3} comprises a CDR-H1 comprising the amino acid sequence of SEQ ID NO:78, a CDR-H2 comprising the amino acid sequence of SEQ ID NO:79, and a CDR-H3 comprising the amino acid sequence of SEQ ID NO:80; and V_{L3} comprises a CDR-L1 comprising the amino acid sequence of SEQ ID NO:81, a CDR-L2 comprising the amino acid sequence of SEQ ID NO:82, and a CDR-L3 comprising the amino acid sequence of SEQ ID NO:83; and wherein:
(a) V_{H3} comprises an amino acid sequence that is at least 95%, at least 96%, at least 97%, at least 98%, at least 99%, or 100% identical to the amino acid sequence of SEQ ID NO:91, and V_{L3} comprises an amino acid sequence that is at least 95%, at least 96%, at least 97%, at least 98%, at least 99%, or 100% identical to the amino acid sequence of SEQ ID NO:92;
(b) V_{H3} comprises an amino acid sequence that is at least 95%, at least 96%, at least 97%, at least 98%, at least 99%, or 100% identical to the amino acid sequence of SEQ ID NO:93, and V_{L3} comprises an amino acid sequence that is at least 95%, at least 96%, at least 97%, at least 98%, at least 99%, or 100% identical to the amino acid sequence of SEQ ID NO:94;
(c) V_{H3} comprises an amino acid sequence that is at least 95%, at least 96%, at least 97%, at least 98%, at least 99%, or 100% identical to the amino acid sequence of SEQ ID NO:95, and V_{L3} comprises an amino acid sequence that is at least 95%, at least 96%, at least 97%, at least 98%, at least 99%, or 100% identical to the amino acid sequence of SEQ ID NO:96; or
(d) V_{H3} comprises an amino acid sequence that is at least 95%, at least 96%, at least 97%, at least 98%, at least 99%, or 100% identical to the amino acid sequence of SEQ ID NO:97, and V_{L3} comprises an amino acid sequence that is at least 95%, at least 96%, at least 97%, at least 98%, at least 99%, or 100% identical to the amino acid sequence of SEQ ID NO:96.

8: The binding protein of claim 1, wherein V_{H1} comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3; V_{L1} comprises a CDR-L1 comprising the amino acid sequence of SEQ ID NO:6, a CDR-L2 comprising the amino acid sequence of SEQ ID NO:8, and a CDR-L3 comprising the amino acid sequence of SEQ ID NO:10; V_{H2} comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59; V_{L2} comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62; V_{H3} comprises a CDR-H1 comprising the amino acid sequence of SEQ ID NO:78, a CDR-H2 comprising the amino acid sequence of SEQ ID NO:79, and a CDR-H3 comprising the amino acid sequence of SEQ ID NO:80; and V_{L3} comprises a CDR-L1 comprising the amino acid sequence of SEQ ID NO:81, a CDR-L2 comprising the amino acid sequence of SEQ ID NO:82, and a CDR-L3 comprising the amino acid sequence of SEQ ID NO:83;
optionally wherein V_{H1} comprises the amino acid sequence of SEQ ID NO:34, V_{L1} comprises the amino acid sequence of SEQ ID NO:35, V_{H2} comprises the amino acid sequence of SEQ ID NO:70, V_{L2} comprises the amino acid sequence of SEQ ID NO:71, V_{H3} comprises the amino acid sequence of SEQ ID NO:91, and V_{L3} comprises the amino acid sequence of SEQ ID NO:92.

9-11. (canceled)

12: The binding protein of claim 1, wherein the first polypeptide chain of the binding protein comprises an amino acid sequence that is at least 95%, at least 96%, at least 97%, at least 98%, at least 99%, or 100% identical to the amino acid sequence of SEQ ID NO:98, the second polypeptide chain of the binding protein comprises an amino acid sequence that is at least 95%, at least 96%, at least 97%, at least 98%, at least 99%, or 100% identical to the amino acid sequence of SEQ ID NO:99, the third polypeptide chain of the binding protein comprises an amino acid sequence that is at least 95%, at least 96%, at least 97%, at least 98%, at least 99%, or 100% identical to the amino acid sequence of SEQ ID NO:100, and the fourth polypeptide chain of the binding protein comprises an amino acid sequence that is at least 95%, at least 96%, at least 97%, at least 98%, at least 99%, or 100% identical to the amino acid sequence of SEQ ID NO:101.

13: A pharmaceutical composition comprising the binding protein of claim 1 and a pharmaceutically acceptable carrier.

14. (canceled)

15: An antibody that specifically binds an OX40L polypeptide, wherein the antibody comprises a heavy chain variable (VH) domain and a light chain variable (VH) domain; wherein the VH domain comprises a CDR-H1 comprising the amino acid sequence of SEQ ID NO:78, a CDR-H2 comprising the amino acid sequence of SEQ ID NO:79, and a CDR-H3 comprising the amino acid sequence of SEQ ID NO:80, and the VL domain comprises a CDR-L1 comprising the amino acid sequence of SEQ ID NO:81, a CDR-L2 comprising the amino acid sequence of SEQ ID NO:82, and a CDR-L3 comprising the amino acid sequence of SEQ ID NO:83;
optionally wherein:
(a) the VH domain comprises an amino acid sequence that is at least 95%, at least 96%, at least 97%, at least 98%, at least 99%, or 100% identical to the amino acid sequence of SEQ ID NO:91, and the VL domain comprises an amino acid sequence that is at least 95%, at least 96%, at least 97%, at least 98%, at least 99%, or 100% identical to the amino acid sequence of SEQ ID NO:92;
(b) the VH domain comprises an amino acid sequence that is at least 95%, at least 96%, at least 97%, at least 98%, at least 99%, or 100% identical to the amino acid sequence of SEQ ID NO:93, and the VL domain comprises an amino acid sequence that is at least 95%, at least 96%, at least 97%, at least 98%, at least 99%, or 100% identical to the amino acid sequence of SEQ ID NO:94;
(c) the VH domain comprises an amino acid sequence that is at least 95%, at least 96%, at least 97%, at least 98%, at least 99%, or 100% identical to the amino acid sequence of SEQ ID NO:95, and the VL domain comprises an amino acid sequence that is at least 95%, at least 96%, at least 97%, at least 98%, at least 99%, or 100% identical to the amino acid sequence of SEQ ID NO:96; or
(d) the VH domain comprises an amino acid sequence that is at least 95%, at least 96%, at least 97%, at least 98%, at least 99%, or 100% identical to the amino acid sequence of SEQ ID NO:97, and the VL domain comprises an amino acid sequence that is at least 95%, at least 96%, at least 97%, at least 98%, at least 99%, or 100% identical to the amino acid sequence of SEQ ID NO:96.

16: A trispecific, trivalent, multispecific and/or multivalent binding protein comprising four polypeptide chains that form three antigen binding sites, wherein a first polypeptide chain of the binding protein comprises a structure represented by the formula: 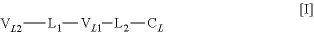
and a second polypeptide chain of the binding protein comprises a structure represented by the formula: 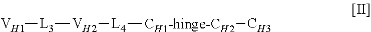
and a third polypeptide chain of the binding protein comprises a structure represented by the formula: 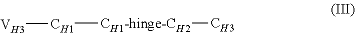
and a fourth polypeptide chain of the binding protein comprises a structure represented by the formula: 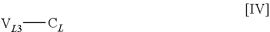
wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are each independently amino acid linkers or zero amino acids in length;
wherein the polypeptide of formula I and the polypeptide of formula II form a cross-over light chain-heavy chain pair of the binding protein;
wherein V_{H1} and V_{L1} form a binding pair and a first antigen binding site, V_{H2} and V_{L2} form a binding pair and a second antigen binding site, and V_{H3} and V_{L3} form a binding pair and a third antigen binding site; and
wherein one of the three antigen binding sites specifically binds an interleukin-17 (IL-17) polypeptide and comprises:
(a) a heavy chain variable (VH) domain that comprises a CDR-H1 comprising the amino acid sequence of SEQ ID NO:117, a CDR-H2 comprising the amino acid sequence of SEQ ID NO:118, and a CDR-H3 comprising the amino acid sequence of SEQ ID NO:119, and a light chain variable (VH) domain that comprises a CDR-L1 comprising the amino acid sequence of SEQ ID NO:114, a CDR-L2 comprising the amino acid sequence of SEQ ID NO:115, and a CDR-L3 comprising the amino acid sequence of SEQ ID NO:116;
(b) a heavy chain variable (VH) domain that comprises a CDR-H1 comprising the amino acid sequence of SEQ ID NO:123, a CDR-H2 comprising the amino acid sequence of SEQ ID NO:124, and a CDR-H3 comprising the amino acid sequence of SEQ ID NO:125, and a light chain variable (VH) domain that comprises a CDR-L1 comprising the amino acid sequence of SEQ ID NO:120, a CDR-L2 comprising the amino acid sequence of GTY, and a CDR-L3 comprising the amino acid sequence of SEQ ID NO:122;
(c) a heavy chain variable (VH) domain that comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131, and a light chain variable (VH) domain that comprises a CDR-L1 comprising the amino acid sequence of SEQ ID NO:126, a CDR-L2 comprising the amino acid sequence of GTY, and a CDR-L3 comprising the amino acid sequence of SEQ ID NO:128;
(d) a VH domain that contains the same CDR-H1 sequence, the same CDR-H2 sequence, and/or the same CDR-H3 sequence, as the amino acid sequence of SEQ ID NO:132 and a VL domain that contains the same CDR-L1 sequence, the same CDR-L2 sequence, and/or the same CDR-L3 sequence, as the amino acid sequence of SEQ ID NO:133;
(e) a VH domain that contains the same CDR-H1 sequence, the same CDR-H2 sequence, and/or the same CDR-H3 sequence, as the amino acid sequence of SEQ ID NO:134 and a V_{L} domain that contains the same CDR-L1 sequence, the same CDR-L2 sequence, and/or the same CDR-L3 sequence, as the amino acid sequence of SEQ ID NO:135; or
(f) a VH domain that contains the same CDR-H1 sequence, the same CDR-H2 sequence, and/or the same CDR-H3 sequence, as the amino acid sequence of SEQ ID NO:136 and a VL domain that contains the same CDR-L1 sequence, the same CDR-L2 sequence, and/or the same CDR-L3 sequence, as the amino acid sequence of SEQ ID NO:137;
optionally wherein the antigen binding site that specifically binds an IL-17 polypeptide is the third antigen binding site formed by V_{H3} and V_{L3}.

17: The binding protein of claim 16, wherein the antigen binding site that specifically binds an IL-17 polypeptide comprises:
(a) a VH domain that comprises an amino acid sequence that is at least 95%, at least 96%, at least 97%, at least 98%, at least 99%, or 100% identical to the amino acid sequence of SEQ ID NO:132 and a VH domain that comprises an amino acid sequence that is at least 95%, at least 96%, at least 97%, at least 98%, at least 99%, or 100% identical to the amino acid sequence of SEQ ID NO:133;
(b) a VH domain that comprises an amino acid sequence that is at least 95%, at least 96%, at least 97%, at least 98%, at least 99%, or 100% identical to the amino acid sequence of SEQ ID NO:134 and a VH domain that comprises an amino acid sequence that is at least 95%, at least 96%, at least 97%, at least 98%, at least 99%, or 100% identical to the amino acid sequence of SEQ ID NO:135; or
(c) a VH domain that comprises an amino acid sequence that is at least 95%, at least 96%, at least 97%, at least 98%, at least 99%, or 100% identical to the amino acid sequence of SEQ ID NO:136 and a VH domain that comprises an amino acid sequence that is at least 95%, at least 96%, at least 97%, at least 98%, at least 99%, or 100% identical to the amino acid sequence of SEQ ID NO:137.

18-20. (canceled)

21: The binding protein of claim 16, wherein:
(a) V_{H1} comprises a CDR-H1 comprising the amino acid sequence of SEQ ID NO:12, a CDR-H2 comprising the amino acid sequence of SEQ ID NO:13, and a CDR-H3 comprising the amino acid sequence of SEQ ID NO:14; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO:123, a CDR-H2 comprising the amino acid sequence of SEQ ID NO:124, and a CDR-H3 comprising the amino acid sequence of SEQ ID NO:125; and V_{L3} comprises a CDR-L1 comprising the amino acid sequence of SEQ ID NO:120, a CDR-L2 comprising the amino acid sequence of GTY, and a CDR-L3 comprising the amino acid sequence of SEQ ID NO:122;
(b) V_{H1} contains the same CDR-H1 sequence, the same CDR-H2 sequence, and/or the same CDR-H3 sequence, as the amino acid sequence of SEQ ID NO:40 and V_{L1} contains the same CDR-L1 sequence, the same CDR-L2 sequence, and/or the same CDR-L3 sequence, as the amino acid sequence of SEQ ID NO:41; V_{H2} contains the same CDR-H1 sequence, the same CDR-H2 sequence, and/or the same CDR-H3 sequence, as the amino acid sequence of SEQ ID NO:72 and V_{L2} contains the same CDR-L1 sequence, the same CDR-L2 sequence, and/or the same CDR-L3 sequence, as the amino acid sequence of SEQ ID NO:73; and V_{H3} contains the same CDR-H1 sequence, the same CDR-H2 sequence, and/or the same CDR-H3 sequence, as the amino acid sequence of SEQ ID NO:134 and V_{L3} contains the same CDR-L1 sequence, the same CDR-L2 sequence, and/or the same CDR-L3 sequence, as the amino acid sequence of SEQ ID NO:135;
(c) V_{H1} comprises a CDR-H1 comprising the amino acid sequence of SEQ ID NO:12, a CDR-H2 comprising the amino acid sequence of SEQ ID NO:13, and a CDR-H3 comprising the amino acid sequence of SEQ ID NO:14; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO:117, a CDR-H2 comprising the amino acid sequence of SEQ ID NO:118, and a CDR-H3 comprising the amino acid sequence of SEQ ID NO:119; and V_{L3} comprises a CDR-L1 comprising the amino acid sequence of SEQ ID NO:114, a CDR-L2 comprising the amino acid sequence of TFF, and a CDR-L3 comprising the amino acid sequence of 116;
(d) V_{H1} contains the same CDR-H1 sequence, the same CDR-H2 sequence, and/or the same CDR-H3 sequence, as the amino acid sequence of SEQ ID NO:40 and V_{L1} contains the same CDR-L1 sequence, the same CDR-L2 sequence, and/or the same CDR-L3 sequence, as the amino acid sequence of SEQ ID NO:41; V_{H2} contains the same CDR-H1 sequence, the same CDR-H2 sequence, and/or the same CDR-H3 sequence, as the amino acid sequence of SEQ ID NO:72 and V_{L2} contains the same CDR-L1 sequence, the same CDR-L2 sequence, and/or the same CDR-L3 sequence, as the amino acid sequence of SEQ ID NO:73; and V_{H3} contains the same CDR-H1 sequence, the same CDR-H2 sequence, and/or the same CDR-H3 sequence, as the amino acid sequence of SEQ ID NO:132 and V_{L3} contains the same CDR-L1 sequence, the same CDR-L2 sequence, and/or the same CDR-L3 sequence, as the amino acid sequence of SEQ ID NO:133;
(e) V_{H1} comprises a CDR-H1 comprising the amino acid sequence of SEQ ID NO:12, a CDR-H2 comprising the amino acid sequence of SEQ ID NO:13, and a CDR-H3 comprising the amino acid sequence of SEQ ID NO:14; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; and V_{L3} comprises a CDR-L1 comprising the amino acid sequence of SEQ ID NO:126, a CDR-L2 comprising the amino acid sequence of GTY, and a CDR-L3 comprising the amino acid sequence of SEQ ID NO:128;
(f) V_{H1} contains the same CDR-H1 sequence, the same CDR-H2 sequence, and/or the same CDR-H3 sequence, as the amino acid sequence of SEQ ID NO:40 and V_{L1} contains the same CDR-L1 sequence, the same CDR-L2 sequence, and/or the same CDR-L3 sequence, as the amino acid sequence of SEQ ID NO:41; V_{H2} contains the same CDR-H1 sequence, the same CDR-H2 sequence, and/or the same CDR-H3 sequence, as the amino acid sequence of SEQ ID NO:72 and V_{L2} contains the same CDR-L1 sequence, the same CDR-L2 sequence, and/or the same CDR-L3 sequence, as the amino acid sequence of SEQ ID NO:73; and V_{H3} contains the same CDR-H1 sequence, the same CDR-H2 sequence, and/or the same CDR-H3 sequence, as the amino acid sequence of SEQ ID NO:136 and V_{L3} contains the same CDR-L1 sequence, the same CDR-L2 sequence, and/or the same CDR-L3 sequence, as the amino acid sequence of SEQ ID NO:137;
(g) V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:31; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO:117, a CDR-H2 comprising the amino acid sequence of SEQ ID NO:118, and a CDR-H3 comprising the amino acid sequence of SEQ ID NO:119; and V_{L3} comprises a CDR-L1 comprising the amino acid sequence of SEQ ID NO:114, a CDR-L2 comprising the amino acid sequence of TFF, and a CDR-L3 comprising the amino acid sequence of SEQ ID NO:116;
(h) V_{H1} contains the same CDR-H1 sequence, the same CDR-H2 sequence, and/or the same CDR-H3 sequence, as the amino acid sequence of SEQ ID NO:53 and V_{L1} contains the same CDR-L1 sequence, the same CDR-L2 sequence, and/or the same CDR-L3 sequence, as the amino acid sequence of SEQ ID NO:41; V_{H2} contains the same CDR-H1 sequence, the same CDR-H2 sequence, and/or the same CDR-H3 sequence, as the amino acid sequence of SEQ ID NO:72 and V_{L2} contains the same CDR-L1 sequence, the same CDR-L2 sequence, and/or the same CDR-L3 sequence, as the amino acid sequence of SEQ ID NO:73; and V_{H3} contains the same CDR-H1 sequence, the same CDR-H2 sequence, and/or the same CDR-H3 sequence, as the amino acid sequence of SEQ ID NO:132 and V_{L3} contains the same CDR-L1 sequence, the same CDR-L2 sequence, and/or the same CDR-L3 sequence, as the amino acid sequence of SEQ ID NO:133;
(i) V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:31; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO:123, a CDR-H2 comprising the amino acid sequence of SEQ ID NO:124, and a CDR-H3 comprising the amino acid sequence of SEQ ID NO:125; and V_{L3} comprises a CDR-L1 comprising the amino acid sequence of SEQ ID NO:120, a CDR-L2 comprising the amino acid sequence of GTY, and a CDR-L3 comprising the amino acid sequence of SEQ ID NO:122;
(j) V_{H1} contains the same CDR-H1 sequence, the same CDR-H2 sequence, and/or the same CDR-H3 sequence, as the amino acid sequence of SEQ ID NO:53 and V_{L1} contains the same CDR-L1 sequence, the same CDR-L2 sequence, and/or the same CDR-L3 sequence, as the amino acid sequence of SEQ ID NO:41; V_{H2} contains the same CDR-H1 sequence, the same CDR-H2 sequence, and/or the same CDR-H3 sequence, as the amino acid sequence of SEQ ID NO:72 and V_{L2} contains the same CDR-L1 sequence, the same CDR-L2 sequence, and/or the same CDR-L3 sequence, as the amino acid sequence of SEQ ID NO:73; and V_{H3} contains the same CDR-H1 sequence, the same CDR-H2 sequence, and/or the same CDR-H3 sequence, as the amino acid sequence of SEQ ID NO:134 and V_{L3} contains the same CDR-L1 sequence, the same CDR-L2 sequence, and/or the same CDR-L3 sequence, as the amino acid sequence of SEQ ID NO:135;
(k) V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:31; V_{L1} comprises a CDR-L1 comprising the amino acid sequence of SEQ ID NO:18, a CDR-L2 comprising the amino acid sequence of DAS, and a CDR-L3 comprising the amino acid sequence of SEQ ID NO:21; V_{H2} comprises a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; V_{L2} comprises a CDR-L1 comprising the amino acid sequence of SEQ ID NO:67, a CDR-L2 comprising the amino acid sequence of KDS, and a CDR-L3 comprising the amino acid sequence of SEQ ID NO:69; V_{H3} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; and V_{L3} comprises a CDR-L1 comprising the amino acid sequence of SEQ ID NO:126, a CDR-L2 comprising the amino acid sequence of GTY, and a CDR-L3 comprising the amino acid sequence of SEQ ID NO:128; or
(l) V_{H1} contains the same CDR-H1 sequence, the same CDR-H2 sequence, and/or the same CDR-H3 sequence, as the amino acid sequence of SEQ ID NO:53 and V_{L1} contains the same CDR-L1 sequence, the same CDR-L2 sequence, and/or the same CDR-L3 sequence, as the amino acid sequence of SEQ ID NO:41; V_{H2} contains the same CDR-H1 sequence, the same CDR-H2 sequence, and/or the same CDR-H3 sequence, as the amino acid sequence of SEQ ID NO:72 and V_{L2} contains the same CDR-L1 sequence, the same CDR-L2 sequence, and/or the same CDR-L3 sequence, as the amino acid sequence of SEQ ID NO:73; and V_{H3} contains the same CDR-H1 sequence, the same CDR-H2 sequence, and/or the same CDR-H3 sequence, as the amino acid sequence of SEQ ID NO:136 and V_{L3} contains the same CDR-L1 sequence, the same CDR-L2 sequence, and/or the same CDR-L3 sequence, as the amino acid sequence of SEQ ID NO:137;
optionally wherein one, two, or all three of the antigen binding sites comprise reciprocal amino acid substitutions on the VH and VL domains that facilitate heavy-light chain pairing; and
optionally wherein:
(i) V_{H1} comprises an amino acid sequence that is at least 95%, at least 96%, at least 97%, at least 98%, at least 99%, or 100% identical to the amino acid sequence of SEQ ID NO:40; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:136; and V_{L3} comprises an amino acid sequence that is at least 95%, at least 96%, at least 97%, at least 98%, at least 99%, or 100% identical to the amino acid sequence of SEQ ID NO:137;
(ii) V_{H1} comprises an amino acid sequence that is at least 95%, at least 96%, at least 97%, at least 98%, at least 99%, or 100% identical to the amino acid sequence of SEQ ID NO:40; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:134; and V_{L3} comprises an amino acid sequence that is at least 95%, at least 96%, at least 97%, at least 98%, at least 99%, or 100% identical to the amino acid sequence of SEQ ID NO:135;
(iii) V_{H1} comprises an amino acid sequence that is at least 95%, at least 96%, at least 97%, at least 98%, at least 99%, or 100% identical to the amino acid sequence of SEQ ID NO:40; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:132; and V_{L3} comprises an amino acid sequence that is at least 95%, at least 96%, at least 97%, at least 98%, at least 99%, or 100% identical to the amino acid sequence of SEQ ID NO:133;
(iv) V_{H1} comprises an amino acid sequence that is at least 95%, at least 96%, at least 97%, at least 98%, at least 99%, or 100% identical to the amino acid sequence of SEQ ID NO:53; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:132; and V_{L3} comprises an amino acid sequence that is at least 95%, at least 96%, at least 97%, at least 98%, at least 99%, or 100% identical to the amino acid sequence of SEQ ID NO:133;
(v) V_{H1} comprises an amino acid sequence that is at least 95%, at least 96%, at least 97%, at least 98%, at least 99%, or 100% identical to the amino acid sequence of SEQ ID NO:53; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:134; and V_{L3} comprises an amino acid sequence that is at least 95%, at least 96%, at least 97%, at least 98%, at least 99%, or 100% identical to the amino acid sequence of SEQ ID NO:135; or
(vi) V_{H1} comprises an amino acid sequence that is at least 95%, at least 96%, at least 97%, at least 98%, at least 99%, or 100% identical to the amino acid sequence of SEQ ID NO:53; V_{L1} comprises an amino acid sequence that is at least 95%, at least 96%, at least 97%, at least 98%, at least 99%, or 100% identical to the amino acid sequence of SEQ ID NO:41; V_{H2} comprises an amino acid sequence that is at least 95%, at least 96%, at least 97%, at least 98%, at least 99%, or 100% identical to the amino acid sequence of SEQ ID NO:72; V_{L2} comprises an amino acid sequence that is at least 95%, at least 96%, at least 97%, at least 98%, at least 99%, or 100% identical to the amino acid sequence of SEQ ID NO:73; V_{H3} comprises an amino acid sequence that is at least 95%, at least 96%, at least 97%, at least 98%, at least 99%, or 100% identical to the amino acid sequence of SEQ ID NO:136; and V_{L3} comprises an amino acid sequence that is at least 95%, at least 96%, at least 97%, at least 98%, at least 99%, or 100% identical to the amino acid sequence of SEQ ID NO:137.

22-25. (canceled)

26: The binding protein of claim 16, wherein:
(a) the first polypeptide chain of the binding protein comprises an amino acid sequence that is at least 95%, at least 96%, at least 97%, at least 98%, at least 99%, or 100% identical to the amino acid sequence of SEQ ID NO:142, the second polypeptide chain of the binding protein comprises an amino acid sequence that is at least 95%, at least 96%, at least 97%, at least 98%, at least 99%, or 100% identical to the amino acid sequence of SEQ ID NO:143, the third polypeptide chain of the binding protein comprises an amino acid sequence that is at least 95%, at least 96%, at least 97%, at least 98%, at least 99%, or 100% identical to the amino acid sequence of SEQ ID NO:144, and the fourth polypeptide chain of the binding protein comprises an amino acid sequence that is at least 95%, at least 96%, at least 97%, at least 98%, at least 99%, or 100% identical to the amino acid sequence of SEQ ID NO:145;
(b) the first polypeptide chain of the binding protein comprises an amino acid sequence that is at least 95%, at least 96%, at least 97%, at least 98%, at least 99%, or 100% identical to the amino acid sequence of SEQ ID NO:138, the second polypeptide chain of the binding protein comprises an amino acid sequence that is at least 95%, at least 96%, at least 97%, at least 98%, at least 99%, or 100% identical to the amino acid sequence of SEQ ID NO:139, the third polypeptide chain of the binding protein comprises an amino acid sequence that is at least 95%, at least 96%, at least 97%, at least 98%, at least 99%, or 100% identical to the amino acid sequence of SEQ ID NO:140, and the fourth polypeptide chain of the binding protein comprises an amino acid sequence that is at least 95%, at least 96%, at least 97%, at least 98%, at least 99%, or 100% identical to the amino acid sequence of SEQ ID NO:141;
(c) the first polypeptide chain of the binding protein comprises an amino acid sequence that is at least 95%, at least 96%, at least 97%, at least 98%, at least 99%, or 100% identical to the amino acid sequence of SEQ ID NO:146, the second polypeptide chain of the binding protein comprises an amino acid sequence that is at least 95%, at least 96%, at least 97%, at least 98%, at least 99%, or 100% identical to the amino acid sequence of SEQ ID NO:147, the third polypeptide chain of the binding protein comprises an amino acid sequence that is at least 95%, at least 96%, at least 97%, at least 98%, at least 99%, or 100% identical to the amino acid sequence of SEQ ID NO:148, and the fourth polypeptide chain of the binding protein comprises an amino acid sequence that is at least 95%, at least 96%, at least 97%, at least 98%, at least 99%, or 100% identical to the amino acid sequence of SEQ ID NO:149;
(d) the first polypeptide chain of the binding protein comprises an amino acid sequence that is at least 95%, at least 96%, at least 97%, at least 98%, at least 99%, or 100% identical to the amino acid sequence of SEQ ID NO:150, the second polypeptide chain of the binding protein comprises an amino acid sequence that is at least 95%, at least 96%, at least 97%, at least 98%, at least 99%, or 100% identical to the amino acid sequence of SEQ ID NO:151, the third polypeptide chain of the binding protein comprises an amino acid sequence that is at least 95%, at least 96%, at least 97%, at least 98%, at least 99%, or 100% identical to the amino acid sequence of SEQ ID NO:152, and the fourth polypeptide chain of the binding protein comprises an amino acid sequence that is at least 95%, at least 96%, at least 97%, at least 98%, at least 99%, or 100% identical to the amino acid sequence of SEQ ID NO:153;
(e) the first polypeptide chain of the binding protein comprises the amino acid sequence of SEQ ID NO:142, the second polypeptide chain of the binding protein comprises the amino acid sequence of SEQ ID NO:143, the third polypeptide chain of the binding protein comprises the amino acid sequence of SEQ ID NO:144, and the fourth polypeptide chain of the binding protein comprises the amino acid sequence of SEQ ID NO:145;
(f) the first polypeptide chain of the binding protein comprises the amino acid sequence of SEQ ID NO:138, the second polypeptide chain of the binding protein comprises the amino acid sequence of SEQ ID NO:139, the third polypeptide chain of the binding protein comprises the amino acid sequence of SEQ ID NO:140, and the fourth polypeptide chain of the binding protein comprises the amino acid sequence of SEQ ID NO:141;
(g) the first polypeptide chain of the binding protein comprises the amino acid sequence of SEQ ID NO:146, the second polypeptide chain of the binding protein comprises the amino acid sequence of SEQ ID NO:147, the third polypeptide chain of the binding protein comprises the amino acid sequence of SEQ ID NO:148, and the fourth polypeptide chain of the binding protein comprises the amino acid sequence of SEQ ID NO:149; or
(h) the first polypeptide chain of the binding protein comprises the amino acid sequence of SEQ ID NO:150, the second polypeptide chain of the binding protein comprises the amino acid sequence of SEQ ID NO:151, the third polypeptide chain of the binding protein comprises the amino acid sequence of SEQ ID NO:152, and the fourth polypeptide chain of the binding protein comprises the amino acid sequence of SEQ ID NO:153.

27: A pharmaceutical composition comprising the binding protein of claim 16 and a pharmaceutically acceptable carrier.

28. (canceled)

29: An antibody that specifically binds an IL-17 polypeptide, wherein the antibody comprises:
(a) a heavy chain variable (VH) domain that comprises a CDR-H1 comprising the amino acid sequence of SEQ ID NO:117, a CDR-H2 comprising the amino acid sequence of SEQ ID NO:118, and a CDR-H3 comprising the amino acid sequence of SEQ ID NO:119, and a light chain variable (VH) domain that comprises a CDR-L1 comprising the amino acid sequence of SEQ ID NO:114, a CDR-L2 comprising the amino acid sequence of TFF, and a CDR-L3 comprising the amino acid sequence of SEQ ID NO:116;
(b) a heavy chain variable (VH) domain that comprises a CDR-H1 comprising the amino acid sequence of SEQ ID NO:123, a CDR-H2 comprising the amino acid sequence of SEQ ID NO:124, and a CDR-H3 comprising the amino acid sequence of SEQ ID NO:125, and a light chain variable (VH) domain that comprises a CDR-L1 comprising the amino acid sequence of SEQ ID NO:120, a CDR-L2 comprising the amino acid sequence of GTY, and a CDR-L3 comprising the amino acid sequence of SEQ ID NO:122;
(c) a heavy chain variable (VH) domain that comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131, and a light chain variable (VH) domain that comprises a CDR-L1 comprising the amino acid sequence of SEQ ID NO:126, a CDR-L2 comprising the amino acid sequence of GTY, and a CDR-L3 comprising the amino acid sequence of SEQ ID NO:128;
(d) a VH domain that contains the same CDR-H1 sequence, the same CDR-H2 sequence, and/or the same CDR-H3 sequence, as the amino acid sequence of SEQ ID NO:132 and a VL domain that contains the same CDR-L1 sequence, the same CDR-L2 sequence, and/or the same CDR-L3 sequence, as the amino acid sequence of SEQ ID NO:133;
(e) a VH domain that contains the same CDR-H1 sequence, the same CDR-H2 sequence, and/or the same CDR-H3 sequence, as the amino acid sequence of SEQ ID NO:134 and a VL domain that contains the same CDR-L1 sequence, the same CDR-L2 sequence, and/or the same CDR-L3 sequence, as the amino acid sequence of SEQ ID NO:135; or
(f) a VH domain that contains the same CDR-H1 sequence, the same CDR-H2 sequence, and/or the same CDR-H3 sequence, as the amino acid sequence of SEQ ID NO:136 and a VL domain that contains the same CDR-L1 sequence, the same CDR-L2 sequence, and/or the same CDR-L3 sequence, as the amino acid sequence of SEQ ID NO:137;
optionally wherein:
(a) the VH domain comprises an amino acid sequence that is at least 95%, at least 96%, at least 97%, at least 98%, at least 99%, or 100% identical to the amino acid sequence of SEQ ID NO:132 and the VL domain comprises an amino acid sequence that is at least 95%, at least 96%, at least 97%, at least 98%, at least 99%, or 100% identical to the amino acid sequence of 133;
(b) the VH domain comprises an amino acid sequence that is at least 95%, at least 96%, at least 97%, at least 98%, at least 99%, or 100% identical to the amino acid sequence of SEQ ID NO:134 and the VL domain comprises an amino acid sequence that is at least 95%, at least 96%, at least 97%, at least 98%, at least 99%, or 100% identical to the amino acid sequence of 135; or
(c) the VH domain comprises an amino acid sequence that is at least 95%, at least 96%, at least 97%, at least 98%, at least 99%, or 100% identical to the amino acid sequence of SEQ ID NO:136 and the VL domain comprises an amino acid sequence that is at least 95%, at least 96%, at least 97%, at least 98%, at least 99%, or 100% identical to the amino acid sequence of 137.

30: The antibody of claim 29, wherein:
the antibody is a monospecific and/or monovalent antibody; or
the antibody is a bispecific and/or bivalent antibody, or a multispecific and/or multivalent antibody.

31. (canceled)

32: A trispecific binding protein comprising four polypeptide chains that form three antigen binding sites, wherein a first polypeptide chain of the binding protein comprises a structure represented by the formula: 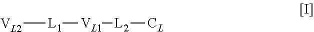
and a second polypeptide chain of the binding protein comprises a structure represented by the formula: 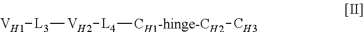
and a third polypeptide chain of the binding protein comprises a structure represented by the formula: 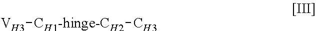
and a fourth polypeptide chain of the binding protein comprises a structure represented by the formula: 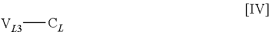
wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L1, L2, L3 and L4 are each independently amino acid linkers or zero amino acids in length;
wherein the polypeptide of formula I and the polypeptide of formula II form a cross-over light chain-heavy chain pair of the binding protein; and
wherein V_{H1} and V_{L1} form a binding pair and a first antigen binding site, V_{H2} and V_{L2} form a binding pair and a second antigen binding site, and V_{H3} and V_{L3} form a binding pair and a third antigen binding site;
wherein one of the three antigen binding sites specifically binds an interleukin-4 (IL-4) polypeptide, one of the three antigen binding sites specifically binds an interleukin-13 (IL-13) polypeptide, and one of the three antigen binding sites specifically binds an NKG2D polypeptide;
optionally wherein the first antigen binding site specifically binds the IL-4 polypeptide and the second antigen binding site specifically binds the IL-13 polypeptide, or the first antigen binding site specifically binds the IL-13 polypeptide and the second antigen binding site specifically binds the IL-4 polypeptide.

33: The binding protein of claim 32, wherein the antigen binding site that specifically binds the NKG2D polypeptide comprises:
(a) a VH domain that comprises a CDR-H1 comprising the amino acid sequence of SEQ ID NO:157, a CDR-H2 comprising the amino acid sequence of SEQ ID NO:158, and a CDR-H3 comprising the amino acid sequence of SEQ ID NO:159, and a VH domain that comprises a CDR-L1 comprising the amino acid sequence of SEQ ID NO:160, a CDR-L2 comprising the amino acid sequence of RDS, and a CDR-L3 comprising the amino acid sequence of SEQ ID NO:162;
(b) a VH domain that comprises a CDR-H1 comprising the amino acid sequence of SEQ ID NO:163, a CDR-H2 comprising the amino acid sequence of SEQ ID NO:164, and a CDR-H3 comprising the amino acid sequence of SEQ ID NO:165, and a VH domain that comprises a CDR-L1 comprising the amino acid sequence of SEQ ID NO:166, a CDR-L2 comprising the amino acid sequence of YDD, and a CDR-L3 comprising the amino acid sequence of SEQ ID NO:168;
(c) a VH domain that comprises a CDR-H1 comprising the amino acid sequence of SEQ ID NO:169, a CDR-H2 comprising the amino acid sequence of SEQ ID NO:170, and a CDR-H3 comprising the amino acid sequence of SEQ ID NO:171, and a VH domain that comprises a CDR-L1 comprising the amino acid sequence of SEQ ID NO:172, a CDR-L2 comprising the amino acid sequence of YDD, and a CDR-L3 comprising the amino acid sequence of SEQ ID NO:174;
(d) a VH domain that comprises a CDR-H1 comprising the amino acid sequence of SEQ ID NO:175, a CDR-H2 comprising the amino acid sequence of SEQ ID NO:176, and a CDR-H3 comprising the amino acid sequence of SEQ ID NO:177, and a VH domain that comprises a CDR-L1 comprising the amino acid sequence of SEQ ID NO:178, a CDR-L2 comprising the amino acid sequence of YDD, and a CDR-L3 comprising the amino acid sequence of SEQ ID NO:180;
(e) a VH domain that comprises a CDR-H1 comprising the amino acid sequence of SEQ ID NO:181, a CDR-H2 comprising the amino acid sequence of SEQ ID NO:182, and a CDR-H3 comprising the amino acid sequence of SEQ ID NO:183, and a VH domain that comprises a CDR-L1 comprising the amino acid sequence of SEQ ID NO:184, a CDR-L2 comprising the amino acid sequence of QDT, and a CDR-L3 comprising the amino acid sequence of SEQ ID NO:186;
(f) a VH domain that comprises a CDR-H1 comprising the amino acid sequence of SEQ ID NO:181, a CDR-H2 comprising the amino acid sequence of SEQ ID NO:182, and a CDR-H3 comprising the amino acid sequence of SEQ ID NO:183, and a VH domain that comprises a CDR-L1 comprising the amino acid sequence of SEQ ID NO:184, a CDR-L2 comprising the amino acid sequence of QDT, and a CDR-L3 comprising the amino acid sequence of SEQ ID NO:186;
(g) a VH domain that comprises a CDR-H1 comprising the amino acid sequence of SEQ ID NO:193, a CDR-H2 comprising the amino acid sequence of SEQ ID NO:194, and a CDR-H3 comprising the amino acid sequence of SEQ ID NO:195, and a VH domain that comprises a CDR-L1 comprising the amino acid sequence of SEQ ID NO:196, a CDR-L2 comprising the amino acid sequence of ATS, and a CDR-L3 comprising the amino acid sequence of SEQ ID NO:198;
(h) a VH domain that comprises a CDR-H1 comprising the amino acid sequence of SEQ ID NO:199, a CDR-H2 comprising the amino acid sequence of SEQ ID NO:200, and a CDR-H3 comprising the amino acid sequence of SEQ ID NO:201, and a VH domain that comprises a CDR-L1 comprising the amino acid sequence of SEQ ID NO:202, a CDR-L2 comprising the amino acid sequence of PAS, and a CDR-L3 comprising the amino acid sequence of SEQ ID NO:204;
(i) a VH domain that comprises a CDR-H1 comprising the amino acid sequence of SEQ ID NO:205, a CDR-H2 comprising the amino acid sequence of SEQ ID NO:206, and a CDR-H3 comprising the amino acid sequence of SEQ ID NO:207, and a VH domain that comprises a CDR-L1 comprising the amino acid sequence of SEQ ID NO:208, a CDR-L2 comprising the amino acid sequence of AAS, and a CDR-L3 comprising the amino acid sequence of SEQ ID NO:210; or
(j) a VH domain that comprises a CDR-H1 comprising the amino acid sequence of SEQ ID NO:211, a CDR-H2 comprising the amino acid sequence of SEQ ID NO:212, and a CDR-H3 comprising the amino acid sequence of SEQ ID NO:213, and a VH domain that comprises a CDR-L1 comprising the amino acid sequence of SEQ ID NO:214, a CDR-L2 comprising the amino acid sequence of YAS, and a CDR-L3 comprising the amino acid sequence of SEQ ID NO:216;
optionally wherein the antigen binding site that specifically binds the NKG2D polypeptide comprises:
(a) a VH domain that comprises an amino acid sequence that is at least 95%, at least 96%, at least 97%, at least 98%, at least 99%, or 100% identical to the amino acid sequence of SEQ ID NO:217 and a VH domain that comprises an amino acid sequence that is at least 95%, at least 96%, at least 97%, at least 98%, at least 99%, or 100% identical to the amino acid sequence of SEQ ID NO:218, optionally V_{H3} comprises an amino acid sequence that is at least 95%, at least 96%, at least 97%, at least 98%, at least 99%, or 100% identical to the amino acid sequence of SEQ ID NO:217 and V_{L3} comprises an amino acid sequence that is at least 95%, at least 96%, at least 97%, at least 98%, at least 99%, or 100% identical to the amino acid sequence of SEQ ID NO:218;
(b) a VH domain that comprises an amino acid sequence that is at least 95%, at least 96%, at least 97%, at least 98%, at least 99%, or 100% identical to the amino acid sequence of SEQ ID NO:219 and a VH domain that comprises an amino acid sequence that is at least 95%, at least 96%, at least 97%, at least 98%, at least 99%, or 100% identical to the amino acid sequence of SEQ ID NO:220, optionally V_{H3} comprises an amino acid sequence that is at least 95%, at least 96%, at least 97%, at least 98%, at least 99%, or 100% identical to the amino acid sequence of SEQ ID NO:219 and V_{L3} comprises an amino acid sequence that is at least 95%, at least 96%, at least 97%, at least 98%, at least 99%, or 100% identical to the amino acid sequence of SEQ ID NO:220;
(c) a VH domain that comprises an amino acid sequence that is at least 95%, at least 96%, at least 97%, at least 98%, at least 99%, or 100% identical to the amino acid sequence of SEQ ID NO:221 and a VH domain that comprises an amino acid sequence that is at least 95%, at least 96%, at least 97%, at least 98%, at least 99%, or 100% identical to the amino acid sequence of SEQ ID NO:222, optionally V_{H3} comprises an amino acid sequence that is at least 95%, at least 96%, at least 97%, at least 98%, at least 99%, or 100% identical to the amino acid sequence of SEQ ID NO:221 and V_{L3} comprises an amino acid sequence that is at least 95%, at least 96%, at least 97%, at least 98%, at least 99%, or 100% identical to the amino acid sequence of SEQ ID NO:222;
(d) a VH domain that comprises an amino acid sequence that is at least 95%, at least 96%, at least 97%, at least 98%, at least 99%, or 100% identical to the amino acid sequence of SEQ ID NO:223 and a VH domain that comprises an amino acid sequence that is at least 95%, at least 96%, at least 97%, at least 98%, at least 99%, or 100% identical to the amino acid sequence of SEQ ID NO:224, optionally V_{H3} comprises an amino acid sequence that is at least 95%, at least 96%, at least 97%, at least 98%, at least 99%, or 100% identical to the amino acid sequence of SEQ ID NO:223 and V_{L3} comprises an amino acid sequence that is at least 95%, at least 96%, at least 97%, at least 98%, at least 99%, or 100% identical to the amino acid sequence of SEQ ID NO:224;
(e) a VH domain that comprises an amino acid sequence that is at least 95%, at least 96%, at least 97%, at least 98%, at least 99%, or 100% identical to the amino acid sequence of SEQ ID NO:225 and a VH domain that comprises an amino acid sequence that is at least 95%, at least 96%, at least 97%, at least 98%, at least 99%, or 100% identical to the amino acid sequence of SEQ ID NO:226, optionally V_{H3} comprises an amino acid sequence that is at least 95%, at least 96%, at least 97%, at least 98%, at least 99%, or 100% identical to the amino acid sequence of SEQ ID NO:225 and V_{L3} comprises an amino acid sequence that is at least 95%, at least 96%, at least 97%, at least 98%, at least 99%, or 100% identical to the amino acid sequence of SEQ ID NO:226;
(f) a VH domain that comprises an amino acid sequence that is at least 95%, at least 96%, at least 97%, at least 98%, at least 99%, or 100% identical to the amino acid sequence of SEQ ID NO:227 and a VH domain that comprises an amino acid sequence that is at least 95%, at least 96%, at least 97%, at least 98%, at least 99%, or 100% identical to the amino acid sequence of SEQ ID NO:228, optionally V_{H3} comprises an amino acid sequence that is at least 95%, at least 96%, at least 97%, at least 98%, at least 99%, or 100% identical to the amino acid sequence of SEQ ID NO:227 and V_{L3} comprises an amino acid sequence that is at least 95%, at least 96%, at least 97%, at least 98%, at least 99%, or 100% identical to the amino acid sequence of SEQ ID NO:228;
(g) a VH domain that comprises an amino acid sequence that is at least 95%, at least 96%, at least 97%, at least 98%, at least 99%, or 100% identical to the amino acid sequence of SEQ ID NO:229 and a VH domain that comprises an amino acid sequence that is at least 95%, at least 96%, at least 97%, at least 98%, at least 99%, or 100% identical to the amino acid sequence of SEQ ID NO:230, optionally V_{H3} comprises an amino acid sequence that is at least 95%, at least 96%, at least 97%, at least 98%, at least 99%, or 100% identical to the amino acid sequence of SEQ ID NO:229 and V_{L3} comprises an amino acid sequence that is at least 95%, at least 96%, at least 97%, at least 98%, at least 99%, or 100% identical to the amino acid sequence of SEQ ID NO:230;
(h) a VH domain that comprises an amino acid sequence that is at least 95%, at least 96%, at least 97%, at least 98%, at least 99%, or 100% identical to the amino acid sequence of SEQ ID NO:231 and a VH domain that comprises an amino acid sequence that is at least 95%, at least 96%, at least 97%, at least 98%, at least 99%, or 100% identical to the amino acid sequence of SEQ ID NO:232, optionally V_{H3} comprises an amino acid sequence that is at least 95%, at least 96%, at least 97%, at least 98%, at least 99%, or 100% identical to the amino acid sequence of SEQ ID NO:231 and V_{L3} comprises an amino acid sequence that is at least 95%, at least 96%, at least 97%, at least 98%, at least 99%, or 100% identical to the amino acid sequence of SEQ ID NO:232;
(i) a VH domain that comprises an amino acid sequence that is at least 95%, at least 96%, at least 97%, at least 98%, at least 99%, or 100% identical to the amino acid sequence of SEQ ID NO:233 and a VH domain that comprises an amino acid sequence that is at least 95%, at least 96%, at least 97%, at least 98%, at least 99%, or 100% identical to the amino acid sequence of SEQ ID NO:234, optionally V_{H3} comprises an amino acid sequence that is at least 95%, at least 96%, at least 97%, at least 98%, at least 99%, or 100% identical to the amino acid sequence of SEQ ID NO:233 and V_{L3} comprises an amino acid sequence that is at least 95%, at least 96%, at least 97%, at least 98%, at least 99%, or 100% identical to the amino acid sequence of SEQ ID NO:234;
(j) a VH domain that comprises an amino acid sequence that is at least 95%, at least 96%, at least 97%, at least 98%, at least 99%, or 100% identical to the amino acid sequence of SEQ ID NO:235 and a VH domain that comprises an amino acid sequence that is at least 95%, at least 96%, at least 97%, at least 98%, at least 99%, or 100% identical to the amino acid sequence of SEQ ID NO:236, optionally V_{H3} comprises an amino acid sequence that is at least 95%, at least 96%, at least 97%, at least 98%, at least 99%, or 100% identical to the amino acid sequence of SEQ ID NO:235 and V_{L3} comprises an amino acid sequence that is at least 95%, at least 96%, at least 97%, at least 98%, at least 99%, or 100% identical to the amino acid sequence of SEQ ID NO:236;
(k) a VH domain that comprises an amino acid sequence that is at least 95%, at least 96%, at least 97%, at least 98%, at least 99%, or 100% identical to the amino acid sequence of SEQ ID NO:237 and a VH domain that comprises an amino acid sequence that is at least 95%, at least 96%, at least 97%, at least 98%, at least 99%, or 100% identical to the amino acid sequence of SEQ ID NO:238, optionally V_{H3} comprises an amino acid sequence that is at least 95%, at least 96%, at least 97%, at least 98%, at least 99%, or 100% identical to the amino acid sequence of SEQ ID NO:237 and V_{L3} comprises an amino acid sequence that is at least 95%, at least 96%, at least 97%, at least 98%, at least 99%, or 100% identical to the amino acid sequence of SEQ ID NO:238;
(l) a VH domain that comprises an amino acid sequence that is at least 95%, at least 96%, at least 97%, at least 98%, at least 99%, or 100% identical to the amino acid sequence of SEQ ID NO:239 and a VH domain that comprises an amino acid sequence that is at least 95%, at least 96%, at least 97%, at least 98%, at least 99%, or 100% identical to the amino acid sequence of SEQ ID NO:238, optionally V_{H3} comprises an amino acid sequence that is at least 95%, at least 96%, at least 97%, at least 98%, at least 99%, or 100% identical to the amino acid sequence of SEQ ID NO:239 and V_{L3} comprises an amino acid sequence that is at least 95%, at least 96%, at least 97%, at least 98%, at least 99%, or 100% identical to the amino acid sequence of SEQ ID NO:238; or
(m) a VH domain that comprises an amino acid sequence that is at least 95%, at least 96%, at least 97%, at least 98%, at least 99%, or 100% identical to the amino acid sequence of SEQ ID NO:240 and a VH domain that comprises an amino acid sequence that is at least 95%, at least 96%, at least 97%, at least 98%, at least 99%, or 100% identical to the amino acid sequence of SEQ ID NO:241, optionally V_{H3} comprises an amino acid sequence that is at least 95%, at least 96%, at least 97%, at least 98%, at least 99%, or 100% identical to the amino acid sequence of SEQ ID NO:240 and V_{L3} comprises an amino acid sequence that is at least 95%, at least 96%, at least 97%, at least 98%, at least 99%, or 100% identical to the amino acid sequence of SEQ ID NO:241.

34: The binding protein of claim 32, wherein:
(1) the antigen binding site that specifically binds the IL-4 polypeptide comprises:
(a) a VH domain that comprises a CDR-H1 comprising the amino acid sequence of SEQ ID NO:1, a CDR-H2 comprising the amino acid sequence of SEQ ID NO:2, and a CDR-H3 comprising the amino acid sequence selected from the group consisting of SEQ ID NOs:3-5, and a VH domain that comprises a CDR-L1 comprising the amino acid sequence of SEQ ID NO:6 or 7, a CDR-L2 comprising the amino acid sequence of SEQ ID NO:8 or 9, and a CDR-L3 comprising the amino acid sequence of SEQ ID NO:10 or 11; or
(b) a VH domain that comprises a CDR-H1 comprising the amino acid sequence of SEQ ID NO:22, a CDR-H2 comprising the amino acid sequence of SEQ ID NO:23, and a CDR-H3 comprising the amino acid sequence selected from the group consisting of SEQ ID NOs:24-27, and a VH domain that comprises a CDR-L1 comprising the amino acid sequence of SEQ ID NO:6 or 7, a CDR-L2 comprising the amino acid sequence of SEQ ID NO:8 or 9, and a CDR-L3 comprising the amino acid sequence of SEQ ID NO:10 or 11;
optionally wherein the binding site that specifically binds an IL-4 polypeptide comprises:
(a) a VH domain that comprises an amino acid sequence that is at least 95%, at least 96%, at least 97%, at least 98%, at least 99%, or 100% identical to the amino acid sequence of SEQ ID NO:34 and a VH domain that comprises an amino acid sequence that is at least 95%, at least 96%, at least 97%, at least 98%, at least 99%, or 100% identical to the amino acid sequence of SEQ ID NO:35, optionally V_{H1} comprises an amino acid sequence that is at least 95%, at least 96%, at least 97%, at least 98%, at least 99%, or 100% identical to the amino acid sequence of SEQ ID NO:34 and V_{L1} comprises an amino acid sequence that is at least 95%, at least 96%, at least 97%, at least 98%, at least 99%, or 100% identical to the amino acid sequence of SEQ ID NO:35;
(b) a VH domain that comprises an amino acid sequence that is at least 95%, at least 96%, at least 97%, at least 98%, at least 99%, or 100% identical to the amino acid sequence of SEQ ID NO:36 and a VH domain that comprises an amino acid sequence that is at least 95%, at least 96%, at least 97%, at least 98%, at least 99%, or 100% identical to the amino acid sequence of SEQ ID NO:37, optionally V_{H1} comprises an amino acid sequence that is at least 95%, at least 96%, at least 97%, at least 98%, at least 99%, or 100% identical to the amino acid sequence of SEQ ID NO:36 and V_{L1} comprises an amino acid sequence that is at least 95%, at least 96%, at least 97%, at least 98%, at least 99%, or 100% identical to the amino acid sequence of SEQ ID NO:37;
(c) a VH domain that comprises an amino acid sequence that is at least 95%, at least 96%, at least 97%, at least 98%, at least 99%, or 100% identical to the amino acid sequence of SEQ ID NO:38 and a VH domain that comprises an amino acid sequence that is at least 95%, at least 96%, at least 97%, at least 98%, at least 99%, or 100% identical to the amino acid sequence of SEQ ID NO:39, optionally V_{H1} comprises an amino acid sequence that is at least 95%, at least 96%, at least 97%, at least 98%, at least 99%, or 100% identical to the amino acid sequence of SEQ ID NO:38 and V_{L1} comprises an amino acid sequence that is at least 95%, at least 96%, at least 97%, at least 98%, at least 99%, or 100% identical to the amino acid sequence of SEQ ID NO:39;
(d) a VH domain that comprises an amino acid sequence that is at least 95%, at least 96%, at least 97%, at least 98%, at least 99%, or 100% identical to the amino acid sequence of SEQ ID NO:40 and a VH domain that comprises an amino acid sequence that is at least 95%, at least 96%, at least 97%, at least 98%, at least 99%, or 100% identical to the amino acid sequence of SEQ ID NO:41, optionally V_{H1} comprises an amino acid sequence that is at least 95%, at least 96%, at least 97%, at least 98%, at least 99%, or 100% identical to the amino acid sequence of SEQ ID NO:40 and V_{L1} comprises an amino acid sequence that is at least 95%, at least 96%, at least 97%, at least 98%, at least 99%, or 100% identical to the amino acid sequence of SEQ ID NO:41;
(e) a VH domain that comprises an amino acid sequence that is at least 95%, at least 96%, at least 97%, at least 98%, at least 99%, or 100% identical to the amino acid sequence of SEQ ID NO:42 and a VH domain that comprises an amino acid sequence that is at least 95%, at least 96%, at least 97%, at least 98%, at least 99%, or 100% identical to the amino acid sequence of SEQ ID NO:43, optionally V_{H1} comprises an amino acid sequence that is at least 95%, at least 96%, at least 97%, at least 98%, at least 99%, or 100% identical to the amino acid sequence of SEQ ID NO:42 and V_{L1} comprises an amino acid sequence that is at least 95%, at least 96%, at least 97%, at least 98%, at least 99%, or 100% identical to the amino acid sequence of SEQ ID NO:43;
(f) a VH domain that comprises an amino acid sequence that is at least 95%, at least 96%, at least 97%, at least 98%, at least 99%, or 100% identical to the amino acid sequence of SEQ ID NO:44 and a VH domain that comprises an amino acid sequence that is at least 95%, at least 96%, at least 97%, at least 98%, at least 99%, or 100% identical to the amino acid sequence of SEQ ID NO:43, optionally V_{H1} comprises an amino acid sequence that is at least 95%, at least 96%, at least 97%, at least 98%, at least 99%, or 100% identical to the amino acid sequence of SEQ ID NO:44 and V_{L1} comprises an amino acid sequence that is at least 95%, at least 96%, at least 97%, at least 98%, at least 99%, or 100% identical to the amino acid sequence of SEQ ID NO:43;
(g) a VH domain that comprises an amino acid sequence that is at least 95%, at least 96%, at least 97%, at least 98%, at least 99%, or 100% identical to the amino acid sequence of SEQ ID NO:45 and a VH domain that comprises an amino acid sequence that is at least 95%, at least 96%, at least 97%, at least 98%, at least 99%, or 100% identical to the amino acid sequence of SEQ ID NO:43, optionally V_{H1} comprises an amino acid sequence that is at least 95%, at least 96%, at least 97%, at least 98%, at least 99%, or 100% identical to the amino acid sequence of SEQ ID NO:45 and V_{L1} comprises an amino acid sequence that is at least 95%, at least 96%, at least 97%, at least 98%, at least 99%, or 100% identical to the amino acid sequence of SEQ ID NO:43;
(h) a VH domain that comprises an amino acid sequence that is at least 95%, at least 96%, at least 97%, at least 98%, at least 99%, or 100% identical to the amino acid sequence of SEQ ID NO:46 and a VH domain that comprises an amino acid sequence that is at least 95%, at least 96%, at least 97%, at least 98%, at least 99%, or 100% identical to the amino acid sequence of SEQ ID NO:47, optionally V_{H1} comprises an amino acid sequence that is at least 95%, at least 96%, at least 97%, at least 98%, at least 99%, or 100% identical to the amino acid sequence of SEQ ID NO:46 and V_{L1} comprises an amino acid sequence that is at least 95%, at least 96%, at least 97%, at least 98%, at least 99%, or 100% identical to the amino acid sequence of SEQ ID NO:47;
(i) a VH domain that comprises an amino acid sequence that is at least 95%, at least 96%, at least 97%, at least 98%, at least 99%, or 100% identical to the amino acid sequence of SEQ ID NO:48 and a VH domain that comprises an amino acid sequence that is at least 95%, at least 96%, at least 97%, at least 98%, at least 99%, or 100% identical to the amino acid sequence of SEQ ID NO:39, optionally V_{H1} comprises an amino acid sequence that is at least 95%, at least 96%, at least 97%, at least 98%, at least 99%, or 100% identical to the amino acid sequence of SEQ ID NO:48 and V_{L1} comprises an amino acid sequence that is at least 95%, at least 96%, at least 97%, at least 98%, at least 99%, or 100% identical to the amino acid sequence of SEQ ID NO:39;
(j) a VH domain that comprises an amino acid sequence that is at least 95%, at least 96%, at least 97%, at least 98%, at least 99%, or 100% identical to the amino acid sequence of SEQ ID NO:49 and a VH domain that comprises an amino acid sequence that is at least 95%, at least 96%, at least 97%, at least 98%, at least 99%, or 100% identical to the amino acid sequence of SEQ ID NO:41, optionally V_{H1} comprises an amino acid sequence that is at least 95%, at least 96%, at least 97%, at least 98%, at least 99%, or 100% identical to the amino acid sequence of SEQ ID NO:49 and V_{L1} comprises an amino acid sequence that is at least 95%, at least 96%, at least 97%, at least 98%, at least 99%, or 100% identical to the amino acid sequence of SEQ ID NO:41;
(k) a VH domain that comprises an amino acid sequence that is at least 95%, at least 96%, at least 97%, at least 98%, at least 99%, or 100% identical to the amino acid sequence of SEQ ID NO:50 and a VH domain that comprises an amino acid sequence that is at least 95%, at least 96%, at least 97%, at least 98%, at least 99%, or 100% identical to the amino acid sequence of SEQ ID NO:43, optionally V_{H1} comprises an amino acid sequence that is at least 95%, at least 96%, at least 97%, at least 98%, at least 99%, or 100% identical to the amino acid sequence of SEQ ID NO:50 and V_{L1} comprises an amino acid sequence that is at least 95%, at least 96%, at least 97%, at least 98%, at least 99%, or 100% identical to the amino acid sequence of SEQ ID NO:43;
(l) a VH domain that comprises an amino acid sequence that is at least 95%, at least 96%, at least 97%, at least 98%, at least 99%, or 100% identical to the amino acid sequence of SEQ ID NO:51 and a VH domain that comprises an amino acid sequence that is at least 95%, at least 96%, at least 97%, at least 98%, at least 99%, or 100% identical to the amino acid sequence of SEQ ID NO:37, optionally V_{H1} comprises an amino acid sequence that is at least 95%, at least 96%, at least 97%, at least 98%, at least 99%, or 100% identical to the amino acid sequence of SEQ ID NO:51 and V_{L1} comprises an amino acid sequence that is at least 95%, at least 96%, at least 97%, at least 98%, at least 99%, or 100% identical to the amino acid sequence of SEQ ID NO:37;
(m) a VH domain that comprises an amino acid sequence that is at least 95%, at least 96%, at least 97%, at least 98%, at least 99%, or 100% identical to the amino acid sequence of SEQ ID NO:52 and a VH domain that comprises an amino acid sequence that is at least 95%, at least 96%, at least 97%, at least 98%, at least 99%, or 100% identical to the amino acid sequence of SEQ ID NO:39, optionally V_{H1} comprises an amino acid sequence that is at least 95%, at least 96%, at least 97%, at least 98%, at least 99%, or 100% identical to the amino acid sequence of SEQ ID NO:52 and V_{L1} comprises an amino acid sequence that is at least 95%, at least 96%, at least 97%, at least 98%, at least 99%, or 100% identical to the amino acid sequence of SEQ ID NO:39;
(n) a VH domain that comprises an amino acid sequence that is at least 95%, at least 96%, at least 97%, at least 98%, at least 99%, or 100% identical to the amino acid sequence of SEQ ID NO:53 and a VH domain that comprises an amino acid sequence that is at least 95%, at least 96%, at least 97%, at least 98%, at least 99%, or 100% identical to the amino acid sequence of SEQ ID NO:41, optionally V_{H1} comprises an amino acid sequence that is at least 95%, at least 96%, at least 97%, at least 98%, at least 99%, or 100% identical to the amino acid sequence of SEQ ID NO:53 and V_{L1} comprises an amino acid sequence that is at least 95%, at least 96%, at least 97%, at least 98%, at least 99%, or 100% identical to the amino acid sequence of SEQ ID NO:41;
(o) a VH domain that comprises an amino acid sequence that is at least 95%, at least 96%, at least 97%, at least 98%, at least 99%, or 100% identical to the amino acid sequence of SEQ ID NO:54 and a VH domain that comprises an amino acid sequence that is at least 95%, at least 96%, at least 97%, at least 98%, at least 99%, or 100% identical to the amino acid sequence of SEQ ID NO:43, optionally V_{H1} comprises an amino acid sequence that is at least 95%, at least 96%, at least 97%, at least 98%, at least 99%, or 100% identical to the amino acid sequence of SEQ ID NO:54 and V_{L1} comprises an amino acid sequence that is at least 95%, at least 96%, at least 97%, at least 98%, at least 99%, or 100% identical to the amino acid sequence of SEQ ID NO:43;
(p) a VH domain that comprises an amino acid sequence that is at least 95%, at least 96%, at least 97%, at least 98%, at least 99%, or 100% identical to the amino acid sequence of SEQ ID NO:55 and a VH domain that comprises an amino acid sequence that is at least 95%, at least 96%, at least 97%, at least 98%, at least 99%, or 100% identical to the amino acid sequence of SEQ ID NO:43, optionally V_{H1} comprises an amino acid sequence that is at least 95%, at least 96%, at least 97%, at least 98%, at least 99%, or 100% identical to the amino acid sequence of SEQ ID NO:55 and V_{L1} comprises an amino acid sequence that is at least 95%, at least 96%, at least 97%, at least 98%, at least 99%, or 100% identical to the amino acid sequence of SEQ ID NO:43; or
(q) a VH domain that comprises an amino acid sequence that is at least 95%, at least 96%, at least 97%, at least 98%, at least 99%, or 100% identical to the amino acid sequence of SEQ ID NO:56 and a VH domain that comprises an amino acid sequence that is at least 95%, at least 96%, at least 97%, at least 98%, at least 99%, or 100% identical to the amino acid sequence of SEQ ID NO:43, optionally V_{H1} comprises an amino acid sequence that is at least 95%, at least 96%, at least 97%, at least 98%, at least 99%, or 100% identical to the amino acid sequence of SEQ ID NO:56 and V_{L1} comprises an amino acid sequence that is at least 95%, at least 96%, at least 97%, at least 98%, at least 99%, or 100% identical to the amino acid sequence of SEQ ID NO:43; and/or
(2) wherein the binding site that specifically binds an IL-13 polypeptide comprises a VH domain that comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59 or 108, and a VH domain that comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62:
optionally wherein the binding site that specifically binds an IL-13 polypeptide comprises:
(a) a VH domain that comprises an amino acid sequence that is at least 95%, at least 96%, at least 97%, at least 98%, at least 99%, or 100% identical to the amino acid sequence of SEQ ID NO:70 and a VH domain that comprises an amino acid sequence that is at least 95%, at least 96%, at least 97%, at least 98%, at least 99%, or 100% identical to the amino acid sequence of SEQ ID NO:71, optionally V_{H2} comprises an amino acid sequence that is at least 95%, at least 96%, at least 97%, at least 98%, at least 99%, or 100% identical to the amino acid sequence of SEQ ID NO:70 and V_{L2} comprises an amino acid sequence that is at least 95%, at least 96%, at least 97%, at least 98%, at least 99%, or 100% identical to the amino acid sequence of SEQ ID NO:71:
(b) a VH domain that comprises an amino acid sequence that is at least 95%, at least 96%, at least 97%, at least 98%, at least 99%, or 100% identical to the amino acid sequence of SEQ ID NO:72 and a VH domain that comprises an amino acid sequence that is at least 95%, at least 96%, at least 97%, at least 98%, at least 99%, or 100% identical to the amino acid sequence of SEQ ID NO:73, optionally V_{H2} comprises an amino acid sequence that is at least 95%, at least 96%, at least 97%, at least 98%, at least 99%, or 100% identical to the amino acid sequence of SEQ ID NO:72 and V_{L2} comprises an amino acid sequence that is at least 95%, at least 96%, at least 97%, at least 98%, at least 99%, or 100% identical to the amino acid sequence of SEQ ID NO:73:
(c) a VH domain that comprises an amino acid sequence that is at least 95%, at least 96%, at least 97%, at least 98%, at least 99%, or 100% identical to the amino acid sequence of SEQ ID NO:74 and a VH domain that comprises an amino acid sequence that is at least 95%, at least 96%, at least 97%, at least 98%, at least 99%, or 100% identical to the amino acid sequence of SEQ ID NO:75, optionally V_{H2} comprises an amino acid sequence that is at least 95%, at least 96%, at least 97%, at least 98%, at least 99%, or 100% identical to the amino acid sequence of SEQ ID NO:74 and V_{L2} comprises an amino acid sequence that is at least 95%, at least 96%, at least 97%, at least 98%, at least 99%, or 100% identical to the amino acid sequence of SEQ ID NO:75: or
(d) a VH domain that comprises an amino acid sequence that is at least 95%, at least 96%, at least 97%, at least 98%, at least 99%, or 100% identical to the amino acid sequence of SEQ ID NO:76 and a VH domain that comprises an amino acid sequence that is at least 95%, at least 96%, at least 97%, at least 98%, at least 99%, or 100% identical to the amino acid sequence of SEQ ID NO:77, optionally V_{H2} comprises an amino acid sequence that is at least 95%, at least 96%, at least 97%, at least 98%, at least 99%, or 100% identical to the amino acid sequence of SEQ ID NO:76 and V_{L2} comprises an amino acid sequence that is at least 95%, at least 96%, at least 97%, at least 98%, at least 99%, or 100% identical to the amino acid sequence of SEQ ID NO:77.

35. (canceled)

36: The binding protein of claim 32, wherein:
(a) V_{H1} comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, V_{L1} comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11, V_{H2} comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, V_{L2} comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62, V_{H3} comprises a CDR-H1 comprising the amino acid sequence of SEQ ID NO:199, a CDR-H2 comprising the amino acid sequence of SEQ ID NO:200, and a CDR-H3 comprising the amino acid sequence of SEQ ID NO:201, and V_{L3} comprises a CDR-L1 comprising the amino acid sequence of SEQ ID NO:202, a CDR-L2 comprising the amino acid sequence of PAS, and a CDR-L3 comprising the amino acid sequence of SEQ ID NO:204, optionally V_{H1} comprises the amino acid sequence of SEQ ID NO:53, V_{L1} comprises the amino acid sequence of SEQ ID NO:41, V_{H2} comprises the amino acid sequence of SEQ ID NO:72, V_{L2} comprises the amino acid sequence of SEQ ID NO:73, V_{H3} comprises the amino acid sequence of SEQ ID NO:240, and V_{L3} comprises the amino acid sequence of SEQ ID NO:241;
(b) V_{H1} comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3, V_{L1} comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11, V_{H2} comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, V_{L2} comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62, V_{H3} comprises a CDR-H1 comprising the amino acid sequence of SEQ ID NO:193, a CDR-H2 comprising the amino acid sequence of SEQ ID NO:194, and a CDR-H3 comprising the amino acid sequence of SEQ ID NO:195, and V_{L3} comprises a CDR-L1 comprising the amino acid sequence of SEQ ID NO:196, a CDR-L2 comprising the amino acid sequence of ATS, and a CDR-L3 comprising the amino acid sequence of SEQ ID NO:198, optionally V_{H1} comprises the amino acid sequence of SEQ ID NO:40, V_{L1} comprises the amino acid sequence of SEQ ID NO:41, V_{H2} comprises the amino acid sequence of SEQ ID NO:72, V_{L2} comprises the amino acid sequence of SEQ ID NO:73, V_{H3} comprises the amino acid sequence of SEQ ID NO:237, and V_{L3} comprises the amino acid sequence of SEQ ID NO:238; or
(c) V_{H1} comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:25, V_{L1} comprises a CDR-L1 comprising the amino acid sequence of SEQ ID NO:7, a CDR-L2 comprising the amino acid sequence of SEQ ID NO:9, and a CDR-L3 comprising the amino acid sequence of SEQ ID NO:11, V_{H2} comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, V_{L2} comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62, V_{H3} comprises a CDR-H1 comprising the amino acid sequence of SEQ ID NO:193, a CDR-H2 comprising the amino acid sequence of SEQ ID NO:194, and a CDR-H3 comprising the amino acid sequence of SEQ ID NO:195, and V_{L3} comprises a CDR-L1 comprising the amino acid sequence of SEQ ID NO:196, a CDR-L2 comprising the amino acid sequence of ATS, and a CDR-L3 comprising the amino acid sequence of SEQ ID NO:198, optionally V_{H1} comprises the amino acid sequence of SEQ ID NO:53, V_{L1} comprises the amino acid sequence of SEQ ID NO:41, V_{H2} comprises the amino acid sequence of SEQ ID NO:72, V_{L2} comprises the amino acid sequence of SEQ ID NO:73, V_{H3} comprises the amino acid sequence of SEQ ID NO:239, and V_{L3} comprises the amino acid sequence of SEQ ID NO:238.

37-40. (canceled)

41: The binding protein of claim 32, wherein:
(a) the first polypeptide chain of the binding protein comprises an amino acid sequence that is at least 95%, at least 96%, at least 97%, at least 98%, at least 99%, or 100% identical to the amino acid sequence of SEQ ID NO:242, the second polypeptide chain of the binding protein comprises an amino acid sequence that is at least 95%, at least 96%, at least 97%, at least 98%, at least 99%, or 100% identical to the amino acid sequence of SEQ ID NO:243, the third polypeptide chain of the binding protein comprises an amino acid sequence that is at least 95%, at least 96%, at least 97%, at least 98%, at least 99%, or 100% identical to the amino acid sequence of SEQ ID NO:244, and the fourth polypeptide chain of the binding protein comprises an amino acid sequence that is at least 95%, at least 96%, at least 97%, at least 98%, at least 99%, or 100% identical to the amino acid sequence of SEQ ID NO:245;
(b) the first polypeptide chain of the binding protein comprises an amino acid sequence that is at least 95%, at least 96%, at least 97%, at least 98%, at least 99%, or 100% identical to the amino acid sequence of SEQ ID NO:246, the second polypeptide chain of the binding protein comprises an amino acid sequence that is at least 95%, at least 96%, at least 97%, at least 98%, at least 99%, or 100% identical to the amino acid sequence of SEQ ID NO:247, the third polypeptide chain of the binding protein comprises an amino acid sequence that is at least 95%, at least 96%, at least 97%, at least 98%, at least 99%, or 100% identical to the amino acid sequence of SEQ ID NO:248, and the fourth polypeptide chain of the binding protein comprises an amino acid sequence that is at least 95%, at least 96%, at least 97%, at least 98%, at least 99%, or 100% identical to the amino acid sequence of SEQ ID NO:249; or
(c) the first polypeptide chain of the binding protein comprises an amino acid sequence that is at least 95%, at least 96%, at least 97%, at least 98%, at least 99%, or 100% identical to the amino acid sequence of SEQ ID NO:250, the second polypeptide chain of the binding protein comprises an amino acid sequence that is at least 95%, at least 96%, at least 97%, at least 98%, at least 99%, or 100% identical to the amino acid sequence of SEQ ID NO:251, the third polypeptide chain of the binding protein comprises an amino acid sequence that is at least 95%, at least 96%, at least 97%, at least 98%, at least 99%, or 100% identical to the amino acid sequence of SEQ ID NO:252, and the fourth polypeptide chain of the binding protein comprises an amino acid sequence that is at least 95%, at least 96%, at least 97%, at least 98%, at least 99%, or 100% identical to the amino acid sequence of SEQ ID NO:253.

42: A pharmaceutical composition comprising the binding protein of claim 32 and a pharmaceutically acceptable carrier.

43. (canceled)

44: An antibody that specifically binds an NKG2D polypeptide, wherein the antibody comprises a heavy chain variable (VH) domain and a light chain variable (VH) domain; and wherein:
(a) the VH domain comprises a CDR-H1 comprising the amino acid sequence of SEQ ID NO:157, a CDR-H2 comprising the amino acid sequence of SEQ ID NO:158, and a CDR-H3 comprising the amino acid sequence of SEQ ID NO:159, and the VL domain comprises a CDR-L1 comprising the amino acid sequence of SEQ ID NO:160, a CDR-L2 comprising the amino acid sequence of RDS, and a CDR-L3 comprising the amino acid sequence of SEQ ID NO:162;
(b) the VH domain comprises a CDR-H1 comprising the amino acid sequence of SEQ ID NO:163, a CDR-H2 comprising the amino acid sequence of SEQ ID NO:164, and a CDR-H3 comprising the amino acid sequence of SEQ ID NO:165, and the VL domain comprises a CDR-L1 comprising the amino acid sequence of SEQ ID NO:166, a CDR-L2 comprising the amino acid sequence of YDD, and a CDR-L3 comprising the amino acid sequence of SEQ ID NO:168;
(c) the VH domain comprises a CDR-H1 comprising the amino acid sequence of SEQ ID NO:169, a CDR-H2 comprising the amino acid sequence of SEQ ID NO:170, and a CDR-H3 comprising the amino acid sequence of SEQ ID NO:171, and the VL domain comprises a CDR-L1 comprising the amino acid sequence of SEQ ID NO:172, a CDR-L2 comprising the amino acid sequence of YDD, and a CDR-L3 comprising the amino acid sequence of SEQ ID NO:174;
(d) the VH domain comprises a CDR-H1 comprising the amino acid sequence of SEQ ID NO:175, a CDR-H2 comprising the amino acid sequence of SEQ ID NO:176, and a CDR-H3 comprising the amino acid sequence of SEQ ID NO:177, and the VL domain comprises a CDR-L1 comprising the amino acid sequence of SEQ ID NO:178, a CDR-L2 comprising the amino acid sequence of YDD, and a CDR-L3 comprising the amino acid sequence of SEQ ID NO:180;
(e) the VH domain comprises a CDR-H1 comprising the amino acid sequence of SEQ ID NO:181, a CDR-H2 comprising the amino acid sequence of SEQ ID NO:182, and a CDR-H3 comprising the amino acid sequence of SEQ ID NO:183, and the VL domain comprises a CDR-L1 comprising the amino acid sequence of SEQ ID NO:184, a CDR-L2 comprising the amino acid sequence of QDT, and a CDR-L3 comprising the amino acid sequence of SEQ ID NO:186;
(f) the VH domain comprises a CDR-H1 comprising the amino acid sequence of SEQ ID NO:187, a CDR-H2 comprising the amino acid sequence of SEQ ID NO:188, and a CDR-H3 comprising the amino acid sequence of SEQ ID NO:189, and the VL domain comprises a CDR-L1 comprising the amino acid sequence of SEQ ID NO:190, a CDR-L2 comprising the amino acid sequence of PAS, and a CDR-L3 comprising the amino acid sequence of SEQ ID NO:192;
(g) the VH domain comprises a CDR-H1 comprising the amino acid sequence of SEQ ID NO:193, a CDR-H2 comprising the amino acid sequence of SEQ ID NO:194, and a CDR-H3 comprising the amino acid sequence of SEQ ID NO:195, and the VL domain comprises a CDR-L1 comprising the amino acid sequence of SEQ ID NO:196, a CDR-L2 comprising the amino acid sequence of ATS, and a CDR-L3 comprising the amino acid sequence of SEQ ID NO:198;
(h) the VH domain comprises a CDR-H1 comprising the amino acid sequence of SEQ ID NO:199, a CDR-H2 comprising the amino acid sequence of SEQ ID NO:200, and a CDR-H3 comprising the amino acid sequence of SEQ ID NO:201, and the VL domain comprises a CDR-L1 comprising the amino acid sequence of SEQ ID NO:202, a CDR-L2 comprising the amino acid sequence of PAS, and a CDR-L3 comprising the amino acid sequence of SEQ ID NO:204;
(i) the VH domain comprises a CDR-H1 comprising the amino acid sequence of SEQ ID NO:205, a CDR-H2 comprising the amino acid sequence of SEQ ID NO:206, and a CDR-H3 comprising the amino acid sequence of SEQ ID NO:207, and the VL domain comprises a CDR-L1 comprising the amino acid sequence of SEQ ID NO:208, a CDR-L2 comprising the amino acid sequence of AAS, and a CDR-L3 comprising the amino acid sequence of SEQ ID NO:210; or
(j) the VH domain comprises a CDR-H1 comprising the amino acid sequence of SEQ ID NO:211, a CDR-H2 comprising the amino acid sequence of SEQ ID NO:212, and a CDR-H3 comprising the amino acid sequence of SEQ ID NO:213, and the VL domain comprises a CDR-L1 comprising the amino acid sequence of SEQ ID NO:214, a CDR-L2 comprising the amino acid sequence of YAS, and a CDR-L3 comprising the amino acid sequence of SEQ ID NO:216;
optionally wherein:
(a) the VH domain comprises an amino acid sequence that is at least 95%, at least 96%, at least 97%, at least 98%, at least 99%, or 100% identical to the amino acid sequence of SEQ ID NO:217 and the VL domain comprises an amino acid sequence that is at least 95%, at least 96%, at least 97%, at least 98%, at least 99%, or 100% identical to the amino acid sequence of SEQ ID NO:218;
(b) the VH domain comprises an amino acid sequence that is at least 95%, at least 96%, at least 97%, at least 98%, at least 99%, or 100% identical to the amino acid sequence of SEQ ID NO:219 and the VL domain comprises an amino acid sequence that is at least 95%, at least 96%, at least 97%, at least 98%, at least 99%, or 100% identical to the amino acid sequence of SEQ ID NO:220;
(c) the VH domain comprises an amino acid sequence that is at least 95%, at least 96%, at least 97%, at least 98%, at least 99%, or 100% identical to the amino acid sequence of SEQ ID NO:221 and the VL domain comprises an amino acid sequence that is at least 95%, at least 96%, at least 97%, at least 98%, at least 99%, or 100% identical to the amino acid sequence of SEQ ID NO:222;
(d) the VH domain comprises an amino acid sequence that is at least 95%, at least 96%, at least 97%, at least 98%, at least 99%, or 100% identical to the amino acid sequence of SEQ ID NO:223 and the VL domain comprises an amino acid sequence that is at least 95%, at least 96%, at least 97%, at least 98%, at least 99%, or 100% identical to the amino acid sequence of SEQ ID NO:224;
(e) the VH domain comprises an amino acid sequence that is at least 95%, at least 96%, at least 97%, at least 98%, at least 99%, or 100% identical to the amino acid sequence of SEQ ID NO:225 and the VL domain comprises an amino acid sequence that is at least 95%, at least 96%, at least 97%, at least 98%, at least 99%, or 100% identical to the amino acid sequence of SEQ ID NO:226;
(f) the VH domain comprises an amino acid sequence that is at least 95%, at least 96%, at least 97%, at least 98%, at least 99%, or 100% identical to the amino acid sequence of SEQ ID NO:227 and the VL domain comprises an amino acid sequence that is at least 95%, at least 96%, at least 97%, at least 98%, at least 99%, or 100% identical to the amino acid sequence of SEQ ID NO:228;
(g) the VH domain comprises an amino acid sequence that is at least 95%, at least 96%, at least 97%, at least 98%, at least 99%, or 100% identical to the amino acid sequence of SEQ ID NO:229 and the VL domain comprises an amino acid sequence that is at least 95%, at least 96%, at least 97%, at least 98%, at least 99%, or 100% identical to the amino acid sequence of SEQ ID NO:230;
(h) the VH domain comprises an amino acid sequence that is at least 95%, at least 96%, at least 97%, at least 98%, at least 99%, or 100% identical to the amino acid sequence of SEQ ID NO:231 and the VL domain comprises an amino acid sequence that is at least 95%, at least 96%, at least 97%, at least 98%, at least 99%, or 100% identical to the amino acid sequence of SEQ ID NO:232;
(i) the VH domain comprises an amino acid sequence that is at least 95%, at least 96%, at least 97%, at least 98%, at least 99%, or 100% identical to the amino acid sequence of SEQ ID NO:233 and the VL domain comprises an amino acid sequence that is at least 95%, at least 96%, at least 97%, at least 98%, at least 99%, or 100% identical to the amino acid sequence of SEQ ID NO:234;
(j) the VH domain comprises an amino acid sequence that is at least 95%, at least 96%, at least 97%, at least 98%, at least 99%, or 100% identical to the amino acid sequence of SEQ ID NO:235 and the VL domain comprises an amino acid sequence that is at least 95%, at least 96%, at least 97%, at least 98%, at least 99%, or 100% identical to the amino acid sequence of SEQ ID NO:236;
(k) the VH domain comprises an amino acid sequence that is at least 95%, at least 96%, at least 97%, at least 98%, at least 99%, or 100% identical to the amino acid sequence of SEQ ID NO:237 and the VL domain comprises an amino acid sequence that is at least 95%, at least 96%, at least 97%, at least 98%, at least 99%, or 100% identical to the amino acid sequence of SEQ ID NO:238;
(l) the VH domain comprises an amino acid sequence that is at least 95%, at least 96%, at least 97%, at least 98%, at least 99%, or 100% identical to the amino acid sequence of SEQ ID NO:239 and the VL domain comprises an amino acid sequence that is at least 95%, at least 96%, at least 97%, at least 98%, at least 99%, or 100% identical to the amino acid sequence of SEQ ID NO:238; or
(m) the VH domain comprises an amino acid sequence that is at least 95%, at least 96%, at least 97%, at least 98%, at least 99%, or 100% identical to the amino acid sequence of SEQ ID NO:240 and the VL domain comprises an amino acid sequence that is at least 95%, at least 96%, at least 97%, at least 98%, at least 99%, or 100% identical to the amino acid sequence of SEQ ID NO:241.

45: A method of preventing and/or treating an inflammatory disease or disorder in a patient in need thereof, comprising administering to the patient a therapeutically effective amount of the pharmaceutical composition of claim 13; optionally wherein the inflammatory disease or disorder is atopic dermatitis; and/or optionally wherein the patient is a human.

46: A method of preventing and/or treating and/or alleviating one or more symptoms of an inflammatory disease or disorder in a patient in need thereof, comprising administering to the patient a therapeutically effective amount of the pharmaceutical composition of claim 27; optionally wherein the inflammatory disease or disorder is atopic dermatitis or chronic obstructive pulmonary disease (COPD); and/or optionally wherein the patient is a human.

47: A method of preventing and/or treating an inflammatory disease or disorder in a patient in need thereof, comprising administering to the patient a therapeutically effective amount of the pharmaceutical composition of claim 42; optionally wherein the inflammatory disease or disorder is atopic dermatitis, Crohn's disease, asthma, or chronic obstructive pulmonary disease (COPD); and/or optionally wherein the patient is a human.

48: An isolated nucleic acid molecule comprising a nucleotide sequence encoding the trispecific binding protein of claim 1.

49: An expression vector comprising the nucleic acid molecule of claim 48.

50: An isolated host cell comprising the nucleic acid molecule of claim 48; optionally wherein the host cell is a mammalian or insect cell.

51: A method of producing a trispecific binding protein, comprising culturing the host cell of claim 50 such that the trispecific binding protein is produced; optionally further comprising recovering the trispecific binding protein from the host cell.

52: An isolated nucleic acid molecule comprising a nucleotide sequence encoding the trispecific, trivalent, multispecific and/or multivalent binding protein of claim 16.

53: An expression vector comprising the nucleic acid molecule of claim 52.

54: An isolated host cell comprising the nucleic acid molecule of claim 52; optionally wherein the host cell is a mammalian or insect cell.

55: A method of producing a trispecific, trivalent, multispecific and/or multivalent binding protein, comprising culturing the host cell of claim 54 such that the trispecific, trivalent, multispecific and/or multivalent binding protein is produced; optionally further comprising recovering the trispecific, trivalent, multispecific and/or multivalent binding protein from the host cell.

56: An isolated nucleic acid molecule comprising a nucleotide sequence encoding the trispecific binding protein of claim 32.

57: An expression vector comprising the nucleic acid molecule of claim 56.

58: An isolated host cell comprising the nucleic acid molecule of claim 56; optionally wherein the host cell is a mammalian or insect cell.

59: A method of producing a trispecific binding protein, comprising culturing the host cell of claim 58 such that the trispecific binding protein is produced; optionally further comprising recovering the trispecific binding protein from the host cell.

60: An antibody that specifically binds an IL-13 polypeptide, wherein the antibody comprises a heavy chain variable (VH) domain and a light chain variable (VH) domain; wherein the VH domain comprises a CDR-H1 comprising the amino acid sequence of SEQ ID NO:57, a CDR-H2 comprising the amino acid sequence of SEQ ID NO:58, and a CDR-H3 comprising the amino acid sequence of SEQ ID NO:59, and the VL domain comprises a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62; optionally wherein:
(a) the VH domain comprises an amino acid sequence that is at least 95%, at least 96%, at least 97%, at least 98%, at least 99%, or 100% identical to the amino acid sequence of SEQ ID NO:70, and the VL domain comprises an amino acid sequence that is at least 95%, at least 96%, at least 97%, at least 98%, at least 99%, or 100% identical to the amino acid sequence of SEQ ID NO:71;
(b) the VH domain comprises an amino acid sequence that is at least 95%, at least 96%, at least 97%, at least 98%, at least 99%, or 100% identical to the amino acid sequence of SEQ ID NO:72, and the VL domain comprises an amino acid sequence that is at least 95%, at least 96%, at least 97%, at least 98%, at least 99%, or 100% identical to the amino acid sequence of SEQ ID NO:73;
(c) the VH domain comprises an amino acid sequence that is at least 95%, at least 96%, at least 97%, at least 98%, at least 99%, or 100% identical to the amino acid sequence of SEQ ID NO:74, and the VL domain comprises an amino acid sequence that is at least 95%, at least 96%, at least 97%, at least 98%, at least 99%, or 100% identical to the amino acid sequence of SEQ ID NO:75; or
(d) the VH domain comprises an amino acid sequence that is at least 95%, at least 96%, at least 97%, at least 98%, at least 99%, or 100% identical to the amino acid sequence of SEQ ID NO:76, and the VL domain comprises an amino acid sequence that is at least 95%, at least 96%, at least 97%, at least 98%, at least 99%, or 100% identical to the amino acid sequence of SEQ ID NO:77.

61: The antibody of claim 60, wherein the antibody is a bispecific and/or bivalent antibody, or a multispecific and/or multivalent antibody.

62: An antibody that specifically binds an IL-4 polypeptide, wherein the antibody comprises a heavy chain variable (VH) domain and a light chain variable (VH) domain; wherein the VH domain comprises:
(a) a CDR-H1 comprising the amino acid sequence of SEQ ID NO:1, a CDR-H2 comprising the amino acid sequence of SEQ ID NO:2, and a CDR-H3 comprising the amino acid sequence selected from the group consisting of SEQ ID NOs:3-5; or
(b) a CDR-H1 comprising the amino acid sequence of SEQ ID NO:22, a CDR-H2 comprising the amino acid sequence of SEQ ID NO:23, and a CDR-H3 comprising the amino acid sequence selected from the group consisting of SEQ ID NOs:24-27; and
wherein the VL domain comprises a CDR-L1 comprising the amino acid sequence of SEQ ID NO:6 or 7, a CDR-L2 comprising the amino acid sequence of SEQ ID NO:8 or 9, and a CDR-L3 comprising the amino acid sequence of SEQ ID NO:10 or 11;
optionally wherein:
(a) the VH domain comprises an amino acid sequence that is at least 95%, at least 96%, at least 97%, at least 98%, at least 99%, or 100% identical to the amino acid sequence of SEQ ID NO:34, and the VL domain comprises an amino acid sequence that is at least 95%, at least 96%, at least 97%, at least 98%, at least 99%, or 100% identical to the amino acid sequence of SEQ ID NO:35;
(b) the VH domain comprises an amino acid sequence that is at least 95%, at least 96%, at least 97%, at least 98%, at least 99%, or 100% identical to the amino acid sequence of SEQ ID NO:36, and the VL domain comprises an amino acid sequence that is at least 95%, at least 96%, at least 97%, at least 98%, at least 99%, or 100% identical to the amino acid sequence of SEQ ID NO:37;
(c) the VH domain comprises an amino acid sequence that is at least 95%, at least 96%, at least 97%, at least 98%, at least 99%, or 100% identical to the amino acid sequence of SEQ ID NO:38, and the VL domain comprises an amino acid sequence that is at least 95%, at least 96%, at least 97%, at least 98%, at least 99%, or 100% identical to the amino acid sequence of SEQ ID NO:39;
(d) the VH domain comprises an amino acid sequence that is at least 95%, at least 96%, at least 97%, at least 98%, at least 99%, or 100% identical to the amino acid sequence of SEQ ID NO:40, and the VL domain comprises an amino acid sequence that is at least 95%, at least 96%, at least 97%, at least 98%, at least 99%, or 100% identical to the amino acid sequence of SEQ ID NO:41;
(e) the VH domain comprises an amino acid sequence that is at least 95%, at least 96%, at least 97%, at least 98%, at least 99%, or 100% identical to the amino acid sequence of SEQ ID NO:42, and the VL domain comprises an amino acid sequence that is at least 95%, at least 96%, at least 97%, at least 98%, at least 99%, or 100% identical to the amino acid sequence of SEQ ID NO:43;
(f) the VH domain comprises an amino acid sequence that is at least 95%, at least 96%, at least 97%, at least 98%, at least 99%, or 100% identical to the amino acid sequence of SEQ ID NO:44, and the VL domain comprises an amino acid sequence that is at least 95%, at least 96%, at least 97%, at least 98%, at least 99%, or 100% identical to the amino acid sequence of SEQ ID NO:43; or
(g) the VH domain comprises an amino acid sequence that is at least 95%, at least 96%, at least 97%, at least 98%, at least 99%, or 100% identical to the amino acid sequence of SEQ ID NO:45, and the VL domain comprises an amino acid sequence that is at least 95%, at least 96%, at least 97%, at least 98%, at least 99%, or 100% identical to the amino acid sequence of SEQ ID NO:43.

63: The antibody of claim 62, wherein the antibody is a bispecific and/or bivalent antibody, or a multispecific and/or multivalent antibody.
